(19) United States
(12) Patent Application Publication
(10) Pub. No.: US 2025/0361488 A1
(43) Pub. Date: Nov. 27, 2025

(54) GENETICALLY ENGINEERED CELLS AND USES THEREOF

(71) Applicants: William Marsh Rice University, Houston, TX (US); The Board of Regents of the University of Texas System, Austin, TX (US)

(72) Inventors: Caleb J. Bashor, Houston, TX (US); Andrew J. Walters, Houston, TX (US); Scott D. Olson, Houston, TX (US)

(21) Appl. No.: 19/204,246

(22) Filed: May 9, 2025

Related U.S. Application Data
(60) Provisional application No. 63/644,937, filed on May 9, 2024.
(60) Provisional application No. 63/660,241, filed on Jun. 14, 2024.

(51) Int. Cl. C12N 5/0775; A61K 35/28; A61P 25/00; C07K 14/195; C07K 14/435; C07K 14/54; C07K 14/705; C07K 14/715; C12N 5/071; C12N 5/074; C12N 9/10; C12N 9/16

(52) U.S. Cl. CPC C12N 5/0662; A61K 35/28; A61P 25/00; C07K 14/195; C07K 14/43595; C07K 14/5437; C07K 14/70503; C07K 14/70546; C07K 14/70553; C07K 14/70596; C07K 14/7158; C12N 5/0686; C12N 5/0696; C12N 9/1051; C12N 9/16; C12Y 301/03001; C12N 2510/00

(57) ABSTRACT

The present invention is directed to a genetically engineered cell and methods thereof as described herein.

This application is a National Phase application which claims the benefit of priority from U.S. provisional patent application No. 63/644,937, filed on May 9, 2024, and U.S. provisional patent application No. 63/660,241, filed on Jun. 14, 2024, the entire contents of each of which are incorporated herein by reference in their entireties.

### GOVERNMENT INTERESTS

This invention was made with government support under Grant No. N00014-21-1-4006, awarded by the Department of Defense, Office of Naval Research, and Grant Nos. R01EB029483, R01EB032272 and R21NS116302, awarded by the National Institutes of Health. The United States government has certain rights in the invention.

All patents, patent applications and publications cited herein are hereby incorporated by reference in their entirety. The disclosures of these publications in their entireties are hereby incorporated by reference into this application in order to more fully describe the state of the art as known to those skilled therein as of the date of the invention described and claimed herein.

This patent disclosure contains material that is subject to copyright protection. The copyright owner has no objection to the facsimile reproduction by anyone of the patent document or the patent disclosure as it appears in the U.S. Patent and Trademark Office patent file or records, but otherwise reserves any and all copyright rights.

### REFERENCE TO A SEQUENCE LISTING XML

This application contains a Sequence Listing which has been submitted electronically in XML format. The Sequence Listing XML is incorporated herein by reference. Said XML file, created on Aug. 12, 2025, is named 2967398-000007-US3_SL.xml and is 426,115 bytes in size.

### FIELD OF THE INVENTION

This invention is directed to a genetically engineered cell. For example, aspects of the invention are drawn to a cell engineered to express one or more of the following: one or more of a cell adhesion protein, one or more of an immunomodulatory protein, one or more of a small molecule inducible safety switch, one or more of a selectable marker, one or more of a reporter, and/or one or more of a cargo.

### BACKGROUND

Cell-based therapies are an emerging pharmaceutical modality that harnesses the intrinsic functional properties of living cells to treat disease. However, many approaches that show therapeutic promise in preclinical studies demonstrate poor or inconsistent outcomes when they are scaled to clinical settings.

### SUMMARY OF THE INVENTION

Aspects of the invention are directed to a genetically engineered cell. In embodiments, the genetically engineered cell is engineered to express one or more modifications. In embodiments, the modifications can comprise one or more of a cell adhesion protein, one or more of an immunomodulatory protein, one or more of a small molecule inducible safety switch, one or more of a selectable marker, one or more of a reporter, and/or one or more of a cargo.

In embodiments, the expression comprises exogenous expression.

In embodiments, the engineered cell is a non-circulatory cell.

In embodiments, the engineered cell comprises a mesenchymal stem/stromal cell (MSC) and its derivates, an induced pluripotent stem cell (iPSC) and its derivatives, a neural stem cell and its derivatives, a hematopoietic stem cell and its derivates, an ARPE-19 cell, or a human embryonic kidney 293 (HEK 293) cell. As used herein, a derivative can be a specialized cell type that has been produced from a stem cell or stromal call through a process called differentiation.

In embodiments, the cell adhesion protein allows for cell homing to a target tissue. For example, the target tissue comprises an inflamed and/or damaged tissue. For example, the tissue comprises neuronal tissue.

In embodiments, the cell adhesion protein comprises a P-selectin ligand, an E-selectin ligand, an L-selectin ligand, or a combination thereof.

In embodiments, the cell expresses a P-selectin ligand, an E-selectin ligand, an L-selectin ligand, or a combination thereof at a level that exceeds the level of expression of a native population of cells.

In embodiments, the cell adhesion proteins comprise PSGL-1, ESL-1, CD44, CD24, PNAd, GlyCAM, CD34, L selectin, ITGB2, ITGAD, ITGAX, ITGAM, PECAM, ITGAL, ITGB1, ITGA9, ITGA4, ITGB7, LFA-1, VLA-4, MAC-1, Talin, Kindlin, FUT7, FUT6, FUT4, ST3GAL4, ST3GAL6, GCNT1, B4GALT1, TPST1, TPST2, ICAM-1, ICAM-2, LPAM-1, JAM-A, JAMB, CXCR1, CXCR2, CXCR3, CXCR4, CXCR5, CXCR6, CXCR7, CCR1, CCR2, CCR3, CCR4, CCR5, CCR6, CCR7, CCR8, CCR9, CCR10, CCR11, CX3CR1, EMR2, EMR3, CD97, MMP-1, MMP-2, MMP-9, TIMP1-3, or any combination thereof.

In embodiments, the immunomodulatory protein comprises CD47, CCL21, CTLA4, CD39, CD73, TRAILR2, PD-L1, PD-L2, FASL, CD40, H2-M3, IL-1R2, MFG-E8, CD200, HLA-G, HLA-E, NQO1, BCL2, Survivin, or any combination thereof.

In embodiments, the selectable marker allows for manufacturing.

In embodiments, the selectable marker comprises puromycin acetyl transferase, truncated CD34, PSGL-1, Blasticidin, or Geneticin.

In embodiments, the reporter comprises iRFP713, luciferase, eBFP2, eGFP, mCherry, or SEAP.

In embodiments, the cargo comprises IL-4, IL-6, IL-10, IL-11, IL-35, IDO1, COX2, TSG-6, TGFB, TNFR2, IFNAR1, GDNF, BDNF, NGF, FGF1-2, NTF3, VEGF, HGF, TGFA, TRAIL, IFNB, HSF-TK, IL-13, IL-12, IL-21, or IL-2.

Aspects of the invention are further drawn to a method of manufacturing the genetically engineered cell as described herein. In embodiments, the method comprises expressing therein one or more of the following: one or more of a cell adhesion protein, one or more of a signal regulatory protein, one or more of a small molecule inducible safety switch, one or more of a selectable marker, one or more of a reporter, and/or one or more of a cargo.

In embodiments, the method comprises isolating and harvesting a cell from a subject or a biological sample.

In embodiments, the method comprises engineering the cell to express one or more of the following: one or more of a cell adhesion protein, one or more of an immune modulatory protein, one or more of a small molecule inducible safety switch, one or more of a selectable marker, one or more of a reporter, and/or one or more of a cargo.

In embodiments, the method further comprises selecting for the cells expressing the proteins.

In embodiments, the method further comprises expanding the isolated cells and/or the genetically engineered cells.

In embodiments, the cell comprises a mesenchymal stem/stromal cell (MSC) and its derivatives, an induced pluripotent stem cell (iPSC) and its derivatives, or a human embryonic kidney 293 (HEK 293) cell. For example, the mesenchymal stem/stromal cell (MSC) is an autologous mesenchymal stem/stromal cell (MSC). For example, the autologous mesenchymal stem/stromal cell (MSC) is a bone marrow-derived mesenchymal stem/stromal cell (MSC) or an adipose tissue-derived mesenchymal stem/stromal cell (MSC). For example, the mesenchymal stem/stromal cell (MSC) is an allogenic mesenchymal stem/stromal cell (MSC). For example, the allogenic mesenchymal stem/stromal cell (MSC) is an umbilical cord-derived mesenchymal stem/stromal cell (MSC), a bone marrow-derived mesenchymal stem/stromal cell (MSC), or an adipose tissue-derived mesenchymal stem/stromal cell (MSC).

Aspects of the invention are further drawn to a method of treating a disease, disorder or medical condition manifesting as inflamed and/or damaged tissue in a subject. In embodiments, the method comprises administering to the subject a population of the genetically engineered cells described herein.

In embodiments, the disease, disorder or medical condition comprises neuroinflammation. For example, the disease, disorder or medical condition comprises traumatic brain injury.

In embodiments, the disease, disorder or medical comprises other diseases characterized by inflammation, including stroke, acute graft vs. host disease, inflammatory bowel disease (e.g., Crohn's disease and/or ulcerative colitis and associated complications), autoimmune diseases, cancer, or any combination thereof.

Other objects and advantages of this invention will become readily apparent from the ensuing description.

### BRIEF DESCRIPTION OF THE FIGURES

The patent or application file contains at least one drawing executed in color. Copies of this patent or patent application publication with color drawing(s) will be provided by the Office upon request and payment of necessary fee.

FIG. 1 provides a schematic showing an overview of the engineered MSC manufacturing process.

FIG. 2 provides data showing optimizations for DNA delivery. Panel A provides data showing the amount of input cells. Panel B provides data showing the amount of input DNA. Panel C provides data showing the effect of increasing DNA size and successful demonstration of delivering>15 kb multigene DNA cargo.

FIG. 3 provides data showing optimizations for cell manufacturing. Panel A provides data showing transposon: transposase ratio. Panel B provides data showing antibiotic selection concentration. Panel C provides data showing plating density. Panel D provides data showing combining modifications results in a cell engineering strategy that produces clinically compatible engineered MSCs. Panel E provides data showing the comparison of various manufacturing strategies. Select=expand cells in antibiotic. Media=select cells for one passage, expand without antibiotic selection. Passage=expand cells without antibiotic selection. Sort=use fluorescence activated cell sorting (FACS) to enrich for transposon and transposase positive cells at 72 hours then expand without antibiotic selection. Panel F provides data showing effect of increasing DNA size and successful demonstration of delivering>15 kb multigene DNA cargo.

FIG. 4 provides a schematic showing applications of MSC cell therapies.

FIG. 5 provides a schematic showing cell adhesion cascade in professional circulatory cells. (Adapted from Vestweber Nat Rev Imm 2015).

FIG. 6 provides a schematic showing an example of how modifying MSCs with a combination of surface proteins from the cell adhesion cascade can improve outcomes in the context of neuroinflammation.

FIG. 7 provides a bar graph showing MSCs expressing PSGL-1 improve adhesion to P&E selectin.

FIG. 8 provides a schematic showing applications of MSC cell therapies.

FIG. 9 provides a schematic showing applications of MSC cell therapies.

FIG. 10 provides a schematic showing challenges with MSC-based therapy.

FIG. 11 provides data showing the framework for cell engineering.

FIG. 12 provides data showing cGMP-compatible engineering. Panel A provides data showing the quantification strategy. Panel B provides data showing the optimized nucleofection protocol. Panel C provides data showing other transfection methods. Panel D provides data showing optimized eMSC manufacturing. Panel E provides data showing eMSC product potency. Panel F provides data showing benchmark to lentivirus. Panel G provides data showing increasing DNA complexity.

FIG. 13 provides data showing multi-gene expression allows for improved adhesion.

FIG. 14 provides data showing engineered secretion enhances potency.

FIG. 15 provides data showing the engineering strategy. Multiparametric optimization results in eMSC numbers above the clinical threshold in only one extra passage (Panel A). The protocol is robust to increasing DNA size by adding expression units. Transient expression shows no significant impact in transfection efficiency or yield. DNA size reduces cell yield when integrated but expandability is unaffected (Panel B).

FIG. 16 provides data showing multi-parametric optimizations. Multi-parametric optimizations spanning various axes of the manufacturing process improve yield of eMSCs when combined.

FIG. 17 provides data showing eMSCs with enhanced targeting. MSCs can be engineered with the surface modifications.

FIG. 18 provides data showing the workflow of design, assembly, characterization, quantification, and comparison of engineered adhesion.

FIG. 19 provides features and constraints of an eMSC manufacturing strategy. Panel A shows eMSC cell therapy product features. Features of a desirable genetically engineered MSC (eMSC) based cell therapy could include multi-gene cargo capacity, highly homogenous transgene expression, long term expression stability, low safety risk, and low cost (left). Multiple approaches have been used pre-clinically and clinically to produce eM-SCs, including non-viral and viral approaches. This representation assumes no selection to homogenize the population nor integration unless inherent to the delivery method (e.g., lentivirus) (right). Panel B shows eMSC manufacturing constraints. An eMSC manufacturing strategy seeks to produce sufficient eMSCs to achieve a pre-defined clinical threshold (orange dotted line) based on indication-specific dosages and tar-get numbers of patients in a trial. A manufacturing run that can produce 10× this threshold from the same starting material is considered to be cost effective (top gray dotted line). This goal is constrained when using donor-derived primary cells by the limited availability of low passage input cells as starting material (bottom gray dotted line) and a restriction on excessive passaging to maintain product safety and avoid senescence (right vertical gray dotted line). As wild type MSCs (black arrow A) exhibit greater than log-linear expansion (4 logs in 3 passages), a pre-determined amount of starting material should expand at least 3 logs in 4 passages to achieve the clinical threshold assuming loss due to the DNA delivery and population homogenization by selection. An ideal eMSC expansion (green arrow B) would expand 4 logs in 4 passages to achieve a cost-effective status. This strategy would be characterized by efficient DNA transfer, near wild type expansion kinetics, and result in a potent, homogenous product (B, right). Error modes in this approach can include a reduced proliferative capacity due to impaired growth from one or more aspects of the transgenesis that results in a low potency, heterogeneous product that may not achieve the clinical threshold (blue arrow C). Additionally, low efficiency DNA delivery can result in bottlenecked expansion that fails to begin WT-like expansion quickly enough to achieve the clinical threshold. Panel C provides a schematic showing eMSC manufacturing workflow. This eMSC manufacturing workflow combines modular DNA parts into a multi-gene array which is nucleofected into human umbilical cord MSCs isolated from donated umbilical cords. eMSCs are produced by selecting and expanding this population of nucleofected cells, then evaluated for optimized performance based on the pre-defined eMSC product features.

FIG. 20 shows optimizing nucleofection. Panel A provides a transient nucleofection workflow to assess GFP reporter construct expression in hUC-MSCs. Nucleofected cells are counted then assayed by flow cytometry to evaluate overall cell yield (black text), nucleofection efficiency (dark green text), and GFP+ yield (light green text). Nucleofected cells are assayed at 48 hours for maximum transient GFP expression. The fraction of the pie chart represents the nucleofection efficiency, while the area of the pie represents overall cell yield (green slice+gray slice) compared to the starting population (black dotted circle). Panel B shows optimizing nucleofection conditions. Using the transient nucleofection workflow, reagent source and preparation, input ratios, and cell handling were independently optimized and represented as a heatmap using normalized GFP+ yield. The condition selected for the optimized nucleofection protocol is shown in orange text. (NEB Stbl.=NEB Stable, Omni=OmniMax, Turbo=NEB Turbo. Q=Qiagen, Z=Zymo) Panel C provides data showing comparison of the optimized nucleofection protocol to manufacturer recommendations (default=50k cells, 1 μg DNA, FF104, AU=arbitrary units) Panel D provides data showing comparison of other transfection methods for overall cell yield (black text), nucleofection efficiency (dark green text), and GFP+ yield (light green text). (Lipo3000=Lipofectamine 3000, LipoLTX=Lipofectamine LTX)

FIG. 21 provides data showing optimizing selection and expansion. Panel A provides data showing eMSC selection and expansion. Using the optimized nucleofection, hUC-MSCs are nucleofected with an integration construct comprising a selectable marker in a piggyBac transposon. The population is selected at 3 d, then assayed at each passage of approximately 7 d each. For this optimization, the target scale was defined as 2 log expansion over 3 passages and started from S=P3. Panel B provides data showing optimizing selection. To optimize selection, the population was assayed after 1 passage (P+1) compared to the starting population (black dotted circle). Transposon: transposase ratio, selection strength, plating density, and electroporation program were evaluated, with the condition selected for the optimized protocol shown in orange text. Panel C provides data showing optimizing expansion. To optimize expansion, the population was assayed over three passages (P+1 to P+3) compared to the target scale (or-ange dotted circle). Various methods to homogenize the population were tested, including constant selection, selection for the first passage only then expansion without selection (pulsed), fluorescence activated cell sorting at 72 hours instead of selection (sort), and no selection (none). The cells were evaluated at each passage for the number of GFP+ cells (overall yield x % GFP positive, light green text in pies), expansion kinetics, and population homogeneity (dark green text, AU=arbitrary units). Values in yield and expansion kinetics plots are average±standard error of the mean (SEM, n=3).

FIG. 22 provides data showing optimized eMSC manufacturing. Panel A provides data showing optimized eMSC manufacturing. The optimized nucleofection, selection, and expansion parameters were combined into a full manufacturing strategy using clinical scale expansion on multi-layer cell culture flasks (top left). The clinical scale expansion was evaluated for overall cell count and expansion kinetics (top middle, dark blue line=WT MSCs, green line=eMSCs), comparison to the target scale (bottom left, orange dotted circle=target scale, light green text=#GFP+ cells), and GFP expression homogeneity across replicates and time course (bottom middle, dark green text=% GFP+, AU=arbitrary units). Yield and expansion kinetics are aver-age±SEM (n=3). Morphology of MSC and eMSC was also assessed (right, scale bar=100 μm). Panel B provides data showing eMSC product potency. Potency of the cell product was evaluated in a battery of immune modulation assays (gray bar=controls without suppression, blue bar=WT MSCs, green bar=eMSCs). TNFα suppression from LPS-activated rat splenocytes (top left, average±SEM, n=3). TNFα suppression from LPS-activated human PBMCs (top middle, average±SEM, n=3-5. 3rd replicate for un-stimulated PBMC had undetectable levels of TNFα). IFNγ suppression from CD3/28-stimulated PBMCs (top right, average±SEM, n=3). T cell proliferation measured as % dividing of various T cell subsets after 48 h of co-culture+/−MSCs or eMSCs (% EdU+=% dividing) (bottom left, average±SEM, n=3). IDO1 induction when MSCs or eMSCs stimulated with hIFNγ via qPCR. IDO1 expression was undetectable without stimulation and is reported as IDO-1 Cq/GAPDH Cq in lieu of 2^{-ΔΔCt} (bottom middle, average±SEM, n=3). PGE2 production when induced by a cytokine cocktail of hTNFα, hIFNγ, and hIFNβ (bottom right, average±SEM, n=3). (SPC=splenocyte, LPS=lipopolysaccharide, PBMC=peripheral blood mononuclear cells, PGE2=prostaglandin E2. *P<0.05, **P<0.005, ***P<0.0005, ****P<0.0001, ns=nonsignificant.)

FIG. 23 provides data showing the evaluation of cell source dependence. Panel A provides data showing cell source dependence. Growth curves for three different donors (circle=do-nor 1, square=donor 2, and triangle=donor 3) from three tissue sources (umbilical cord, adipose tissue, and bone marrow), (S=P2, dark blue line=wild type MSCs, green line=eMSCs). The target cell count represents a 3-log expansion across four passages. The expansion for bone marrow donor 1 (BM1) was terminated after three passages due to insufficient cell numbers and was not assessed for immune modulation. Panel B provides data showing expansion kinetics (top, middle) and yield (bottom) for the MSCs and eMSCs from various donors and tissue sources (orange dotted circle=target scale, light green text=#GFP+ cells, dark green text=% GFP+). Panel C provides data showing potency of the P+3 product as measured in a TNFα suppression assay in co-culture with LPS-activated rat splenocytes (gray bar=controls without suppression, blue bar=WT MSCs, green bar=eM-SCs. Average±SEM, n=3). BM2 was only assessed as a WT cells due to insufficient P+3 or P+4 eMSCs. BM3 was assessed as P+3 for the WT cells and P+4 for WT and eMSCs due to insufficient P+3 eMSCs. (SPC=splenocyte, LPS=lipopolysaccharide. *P<0.05, **P<0.005, ***P<0.0005, ****P<0.0001, ns=nonsignificant)

FIG. 24 provides data showing comparison to lentiviral transduction. Panel A shows the lentiviral transduction workflow. A transfer vector encoding GFP and a selectable marker is packaged in lentivirus and then transduced in hUC-MSCs. The population is selected at 72 h, then expanded for 3 passages from S=P3 cells. Panel B provides data showing the efficiency of lentiviral transduction. Transduction efficiency was assessed at 48 hours before selection (MOI=multiplicity of infection, black dotted circle=starting population, black text=overall yield, dark green=transduction or nucleofection efficiency, light green=#GFP+ cells). Panel C provides data showing the efficiency of lentiviral expansion. Expansion efficiency was assessed over three passages for cell count and expansion kinetics (orange dotted line or circle=target expansion of 2 logs in three passages, dark green text=% GFP+, light green text=#GFP+ cells). Panel D provides data showing lentiviral expansion homogeneity. GFP expression homogeneity for eMSCs transduced at an MOI of 10 or nucleofected according to the optimized protocol (AU=arbitrary units).

FIG. 25 shows the impact of DNA size. Panel A provides a schematic showing the testing of cargo size dependence. Impact of DNA cargo size was assayed by adding additional EUs encoding fluorescent proteins (FPs) to the multi-gene array. DNA cargo size is defined as the number of base pairs between the ITRs, inclusive. Panel B provides data showing size dependence for nucleofection. Nucleofection efficiency (dark green) and FP+ yield (light green) assayed in a transient context at 48 h (gray solid line=linear regression). Panel C provides data showing size dependence for selection/expansion. The number of eMSCs (light green) and the population doublings (black) after selection and one passage of expansion (P+2), (orange dotted line or circle=target expansion of 1 log in two passages, dark green text=% GFP+, light green text=#GFP+ cells, gray solid line=linear regression).

FIG. 26 provides data showing the engineering of IL-10 expressing eMSCs. Panel A provides a schematic showing the engineering of modular and tunable secretion. eMSCs co-cultured with rat splenocytes (SPCs) encoding IL-10 expressing programs or control programs (high=CMV, med=hEF1αV1, low=mPGKV2). Panel B provides data showing the expansion of IL-10 cells. Expansion and growth kinetics for the various programs (S=P2, orange dotted line or circle=target expansion of 2 logs in three passages, dark red text=% iRFP+, light red text=#iRFP+ cells, average±SEM, n=3). Panel C provides data showing IL-10 secretion from the various programs after 48 h (ND=not detected, average±SEM, n=3). Panel D provides data showing suppression of TNFα from co-culture of IL-10 expressing eMSCs with LPS-activated rat splenocytes after 24 h (average±SEM, n=3). Panel E provides data showing the correlation between hIL-10 from IL-10 expressing eMSCs and rTNFα from rat SPCs after 24 h (triangle=high, square=med, circle=low, solid gray line=linear regression). (SPC=splenocyte, LPS=lipopolysaccharide. *P<0.05, **P<0.005, ***P<0.0005, ****P<0.0001)

FIG. 27 provides the cloning workflow used in this study. Multi-level hierarchical cloning workflow for constructing the plasmids described in Table 3. Level 1 assemblies combined PCR-amplifed DNA amplicons of parts (e.g., promoter, ORFs, and terminators) with a linear PCR-amplifed backbone encoding ampicillin resistance (AmpR) to produce individual “parts” plasmids using BsaI (red). These parts plasmids were combined into expression units (EUs) comprising a promoter, ORF, and terminator with a kanamycin resistance (KanR) destination vector containing a dual ccdB-mCherry2 expression cassette using Esp3I (blue). This assembly replaces the ccdB-mCherry2 with the EU. Multigene cassettes were constructed by assembling one or more EUs into a custom piggyBac transposon destination vector that encoded puromycin resistance and contained the dual ccdB-mCherry2 expression cassette using BbsI/BpiI (pink). Greek and Roman letters represent custom overhangs for type IIS assembly. Arrows on the vectors show the orientation of the type IIS sites, while the offset bracket indicates the arrangement of sticky ends left by the enzyme.

FIG. 28 provides an example workflow overview and analysis. A representation of a typical transient experiment. A known number of input cells are nucleofected with a reporter construct, then measured at 48 hours. The cells are counted, the population is phenotyped using flow cytometry. Events were gated by forward (FSC-A) and side scatter area (SSC-A) to yield a population of cells. Single cells were gated by forward scatter height (FSC-H) vs area (FSC-A). The percentage of GFP+ single cells was then recorded as the nucleofection efficiency. Overall yield was calculated from the counted number of cells harvested divided by the known input number of cells. The GFP+ yield was then calculated by multiplying the overall yield by the nucleofection efficiency. These values are reported as bar or line plots, or converted to a pie chart.

FIG. 29 shows the reagent and source material preparation. hUCM-SCs were nucleofected using the eGFP reporter construct then overall yield, nucleofection efficiency, GFP+ yield, and eGFP expression distribution were measured. Input number of cells and amount of DNA were constant within each experiment. Panel A provides data showing MSC preparation. MSCs nucleofected either directly from frozen or after being refreshed in cell culture for 24 hr. Panel B provides data showing MSCs nucleofected with DNA prepared from different E. coli strains. Panel C provides data showing MSCs nucleofected with DNA prepared using various DNA isolation kits. neg=shocked without DNA, Q=Qiagen, Z=Zymo.

FIG. 30 provides data showing optimizing ratios of input materials. MSCs were nucleofected using the reporter construct then overall yield, nucleofection efficiency, GFP+yield, and eGFP expression distribution were measured. The volume of P1 buffer was kept constant within each experiment. Panel A provides data showing the amount of DNA was varied while keeping cell number and volume TE buffer volume constant. Panel B provides data showing the volume of TE buffer was varied while keeping amount of DNA, and cell number constant. Panel C provides data showing the number of input cells was varied while keeping amount of DNA and volume of TE buffer constant. neg=shocked with-out DNA.

FIG. 31 shows testing various handling conditions. MSCs were nucleofected using the reporter construct then overall yield, nucleofection efficiency, GFP+ yield, and eGFP expression distribution were measured. Input number of cells and amount of DNA were constant within each experiment. Panel A provides data showing three electroporation programs that were tested. FF=FF104, recommended as “high efficiency”. EW=EW104, recommended as “high viability”. EA=EA100, a program not recommended for MSCs as a negative control. NS=no shock. ND=no DNA, shocked with FF104. Panel B provides data showing time in buffer. Time in P1 buffer was tested to evaluate toxicity. neg=shocked without DNA. Panel C provides data showing various recovery conditions that were tested, including two temperatures (25° C. and 37° C.), immediate addition of 100 μL media after nucleofection then a 10 min recovery (+media), addition of 100 μL media after a 10 min recovery (-media), or immediate addition of 100 μL media and transfer to cell culture without a 10 min recovery (NR=no recovery).

FIG. 32 provides data showing alternative transfection methods. MSCs were transfected with various chemical transfection methods using the reporter construct then overall yield, nucleofection efficiency, GFP+ yield, and eGFP expression distribution were measured. Input number of cells and amount of DNA were constant within each experiment. Panel A shows Lipofectamine 3000 and Panel B shows Lipofectamine LTX dose response curve with and without dexamethasone treatment. Panel C shows Fugene 4K dose response curve. Panel D shows transfection using GFP mRNA from two different suppliers (System Biosciences and TriLink) and with various ratios of ug RNA to μL Lipofect-amine RNAiMAX.

FIG. 33 provides data showing additions to nucleofection. MSCs were nucleofected using the reporter construct then overall yield, nucleofection efficiency, GFP+ yield, and eGFP expression distribution were measured. Input number of cells and amount of DNA were constant within each experiment. Impact of (Panel A) dexamethasone or (Panel B) CEPT treatment on nucleofection. neg=shocked without DNA.

FIG. 34 shows reproducibility of nucleofection with two different Nucleofector devices. MSCs were nucleofected using either an amaxa 2b with 96 well shuttle or a Lonza 4D nucleofector using the reporter construct, then overall yield and eGFP expression distribution were measured. Input number of cells and amount of DNA were constant.

FIG. 35 shows selection optimizations. MSCs were nucleofected using the integration construct and hyperactive piggyBac transposase, selected at 72 hours, then expanded until the most dense well reached the target confluency. GFP+ yield and eGFP expression distribution were measured. Input number of cells and amount of DNA were constant within each experiment. Panel A provides data showing the ratio of transposon to transposase was varied. Panel B provides data showing the electroporation program was varied. FF=FF104, EW=EW104. Panel C provides data showing the concentration of puromycin was varied. Panel D provides data showing the initial plating density after nucleofection was varied based on the original number of cells before nucleofection. neg=shocked without DNA and no selection.

FIG. 36 provides an example cell engineering time course. MSCs were nucleofected using the integration construct and hyperactive piggyBac transposase. Some cells are lost due to nucleofection and have a broad expression peak (1.5 d). The cells recover but have reduced transient expression (3 d). Cell numbers are mostly consistent during selection, as cell death matches cell division (3-8 d). After reaching the target confluency, the cells are passaged. eGFP expression has stabilized (8 d). Exponential growth occurs after this point, with stable expression over multiple passages (14 and 19 d).

FIG. 37 provides data showing cell source expression. eGFP expression distribution for various cell sources and donors is shown. Gray=WT MSCs, green=eMSCs.

FIG. 38 provides data showing lentivirus testing and manufacturing in HEK 293T cells. Panel A shows Comparison of reporter and integration constructs to the lentivirus constructs tested (left). Restriction enzyme digest confirming lentiviral constructs (right). Panel B provides data showing HEK 293T cells transfected with 1 μg of each transfer vector to test expression. Panel C provides data showing HEK 293T cells transduced with 1 mL viral supernatant to confirm transduction. Panel D provides data showing eGFP expression distribution of HEK 293T cells transfected with R3 transfer vector for viral manufacturing. Panel E provides data showing titration of R3 lentiviral supernatant from Panel D by transducing HEK 293T cells (left) and calculation of viral particles per μL (right). Panel F provides data showing conversion of titration from Panel E to multiplicity of infection (MOI).

FIG. 39 provides data showing lentiviral transduction of MSCs. MSCs were transduced using the R3 lentiviral construct. Input number of cells were constant within each experiment. (Panel A) Overall yield, nucleofection efficiency, GFP+ yield, and eGFP expression distribution were measured at 72 hours before selection and (Panel B) after selection and expansion of cells transduced with MOIs that achieved near 100% yield before selection (10, 20, 50).

FIG. 40 provides data showing transgene expression from multi-gene cassettes. MSCs were nucleofected with various single- and multi-gene cassettes. Input number of cells were consistent within each experiment. Panel A shows transient transfection of single- and multi-gene cassettes. Panel B shows transgene expression of multi-gene cassettes after one passage of selection. Hyperactive piggyBac transposase was co-nucleofected for stable expression. Geo. mean=geometric mean

FIG. 41 provides data showing characterization of the impact of parts on expression level and yield. MSCs were nucleofected with multi-gene cassettes. Input number of cells were consistent within each experiment. Panel A provides an expression construct with independently modular promoters and terminators expressing GFP and a constitutive iRFP reporter for normalization. Hyperactive piggyBac transposase was co-nucleofected for stable expression. Panel B provides data showing the rank order expression of various promoters gated on iRFP expression. Panel C provides data showing the impact of various terminators on expression level. Panel D provides data showing the impact of expression level on yield. eGFP-A and iRFP713-A are presented as geometric means in arbitrary units (AU).

FIG. 42 provides data showing the characterization of IL-10 eMSCs. MSCs were nucleofected with various single- and multi-gene cassettes. Input number of cells were consistent within each experiment. high=CMV, med=hEF1αV1, low=mPGKV2. (Panel A) Relative expression strengths of promoters used in this study were characterized using eGFP reporter constructs with the respective promoters via transient transfection (left). Transient expression of IL-10 from these promoters (middle). Timecourse of IL-10 expression from these promoters (right). (Panels B-C) Product features of IL-10 expressing eMSCs including iRFP expression from the constant reporter (Panel B) and final cell count (Panel C). (Panel D) Rat TNFα suppression in co-culture assay with WT MSCs and eMSCs. SPC=splenocyte, LPS=lipopolysaccharide. *P<0.05, **P<0.005, ***P<0.0005, ****P<0.0001, ns=nonsignificant.

FIG. 43 provides data showing the engineerable platform and scalable assembly line described herein.

FIG. 44 provides data showing leveraging synthetic biology to overcome challenges with MSCs. Panel A provides a schematic showing poor targeting and improved adhesion. Panel B provides data showing multigene synergy using an in vitro adhesion assay. Panel C provides data showing targeting to the spleen in vivo.

FIG. 45 provides data showing leveraging synthetic biology to overcome challenges with MSCs. Panel A provides a schematic showing insufficient potency and custom cargo. Panel B provides data showing suppression of inflammation in vitro. Panel C provides data showing macrophage activation in the spleen. Panel D provides data showing regulatory T-cells in the blood.

FIG. 46 provides a schematic for treating a TBI (traumatic brain injury) model using eMSCs.

FIG. 47 provides data showing in vivo experiment with FUT7+PSGL-1 eMSCs after TBI. Panel A provides a schematic showing constructs P138 and P199. Panel B provides a schematic showing the expansion of eMSCs programmed with P99 and infused in a rat model of traumatic brain injury via tail vein injection. Panel C provides data showing the number of eMSCs in the brain. Panel D provides data showing the number of eMSCs in the spleen. Panel E provides data showing the number of eMSCs in the lungs.

FIG. 48 provides data showing the expression of LFA-1 eMSCs (Integrin aL and B2) and the effect on eMSC adhesion to ICAM-1.

FIG. 49 provides data showing migration and expression of eMSCs using various modifications. Panel A provides data showing migration to SDF-1 using a CXCR4 modification. Panel B provides data showing migration to CCL19 using a CCR7 modification. Panel C provides data showing the combination of L-selectin and CCR7. Panel D provides data showing adhesion using a ST3GAL4 modification to the PSGL-1+FUT7 pair. Panel E provides data showing expression of CD47 in vivo. Panel F provides data showing the CD63-eGFP modification results in cargo loading into extracellular vesicles. Panel G provides data showing tunable secretion of IL-4.

FIG. 50 provides data showing the manufacturing platform used to evaluate synthetic circuits for manufacturability.

### DETAILED DESCRIPTION OF THE INVENTION

Detailed descriptions of one or more embodiments are provided herein. It is to be understood, however, that the present invention may be embodied in various forms. Therefore, specific details disclosed herein are not to be interpreted as limiting, but rather as a basis for the claims and as a representative basis for teaching one skilled in the art to employ the present invention in any appropriate manner.

The singular forms “a”, “an” and “the” include plural reference unless the context clearly dictates otherwise. The use of the word “a” or “an” when used in conjunction with the term “comprising” in the claims and/or the specification may mean “one,” but it is also consistent with the meaning of “one or more,” “at least one,” and “one or more than one.”

Wherever any of the phrases “for example,” “such as,” “including” and the like are used herein, the phrase “and without limitation” is understood to follow unless explicitly stated otherwise. Similarly, “an example,” “exemplary” and the like are understood to be nonlimiting.

The term “substantially” allows for deviations from the descriptor that do not negatively impact the intended purpose. Descriptive terms are understood to be modified by the term “substantially” even if the word “substantially” is not explicitly recited.

The terms “comprising” and “including” and “having” and “involving” (and similarly “comprises”, “includes,” “has,” and “involves”) and the like are used interchangeably and have the same meaning. Specifically, each of the terms is defined consistent with the common United States patent law definition of “comprising” and is therefore interpreted to be an open term meaning “at least the following,” and is also interpreted not to exclude additional features, limitations, aspects, etc. Thus, for example, “a process involving steps a, b, and c” means that the process includes at least steps a, b and c. Wherever the terms “a” or “an” are used, “one or more” is understood, unless such interpretation is nonsensical in context.

The term “about” can refer to approximately, roughly, around, or in the region of. When the term “about” is used in conjunction with a numerical range, it modifies that range by extending the boundaries above and below the numerical values set forth. In general, the term “about” is used herein to modify a numerical value above and below the stated value by a variance of 20 percent up or down (higher or lower).

Aspects of the invention are drawn to a genetically engineered cell engineered to express (i.e., exogenously express) one or more proteins, thereby providing a genetically engineered cell with a non-native protein profile. For example, the one or more proteins can comprise a cell adhesion protein, one or more of an immune modulatory or survival protein, one or more of a small molecule inducible safety switch, one or more of a selectable marker, one or more of a reporter, and/or one or more of a cargo. Without wishing to be bound by theory, embodiments as described herein overcome problems associated with mesenchymal/multipotent/medicinal stem/stromal/signaling cell (MSC) based cell therapy. For example, embodiments described herein address poor targeting of MSCs to desired location by adding surface markers to the cells that allow them to home to inflammation; address poor potency of the cell product by adding indication-specific therapeutic cargo (such as IL-10); address poor persistence in the patient by adding immunomodulatory factors and survival genes to avoid the MSCs from being removed from the body, or some combination thereof.

The term “genetically engineered cell” can refer to a cell whose genetic material has been modified through recombinant DNA technology, gene editing, or other synthetic biology methods to confer a specific trait or function.

In embodiments, for example, the genetically engineered cell expresses a non-native surface profile of proteins that is characteristic of a specific cell type. In one example, the genetically engineered cell can express a non-native surface profile of proteins that results in an emergent homing behavior.

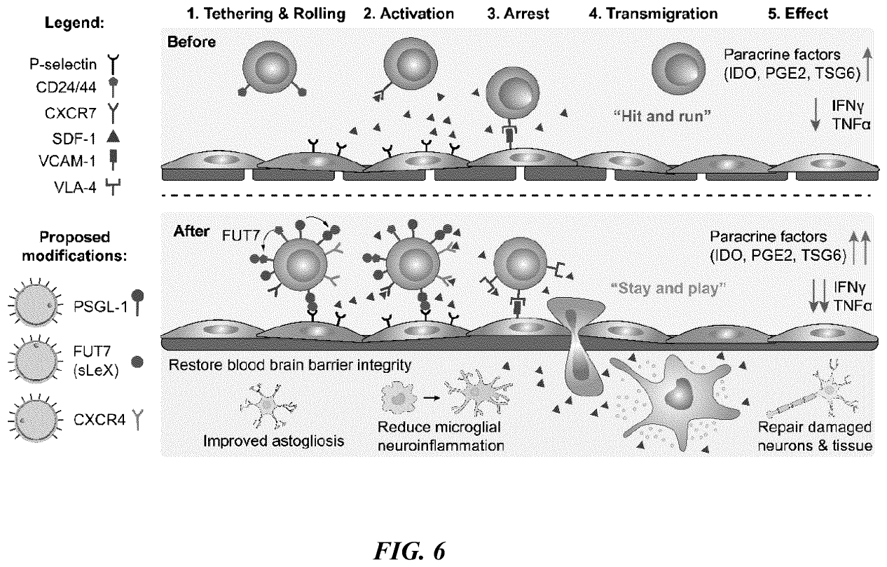

As exemplified in FIG. 6, modifying non-circulatory cells (such as by expressing PSGL-1, FUT7, CXCR4) with a combination of surface proteins from the cell adhesion cascade can improve outcomes in the context of inflammation.

In embodiments, the engineered cell can be a eukaryotic cell. For example, the cell can be a mammalian cell. For example, the cell can be a human cell. Non-limiting examples of cells as utilized herein can include a mesenchymal/multipotent/medicinal stem/stromal/signaling cell (MSC) and its derivates, an induced pluripotent stem cell (iPSC) and its derivatives (e.g. an iPSC derived MSC), a human embryonic kidney 293 (HEK 293), an ARPE-19 cell, a neural stem cell (NSC) and its derivatives, or a hematopoietic stem cell and its derivatives.

In particular embodiments, the engineered cell can comprise a mesenchymal stem/stromal cell. Mesenchymal stem/stromal cells (MSCs) are spindle shaped plastic-adherent cells isolated from bone marrow, adipose, and other tissue sources, with multipotent differentiation capacity in vitro. MSCs are among the most responsive cell populations to inflammation in tissues. MSCs possess stemness, as defined by their ability to differentiate into osteoblasts, adipocytes, and chondrocytes. These cells are present in almost all tissues and are highly mobile in response to tissue damage signals.

In embodiments, the engineered cell can be a stem cell. Stem cells can be distinguished from other cell types by two important characteristics. First, stem cells are unspecialized cells that can renew themselves through cell division, sometimes after long periods of inactivity. Second, under certain physiologic or experimental conditions, stem cells can be induced to become tissue- or organ-specific cells with special functions. In some organs, such as the gut and bone marrow, stem cells regularly divide to repair and replace worn out or damaged tissues. In other organs, however, such as the pancreas and the heart, stem cells only divide under special conditions.

In particular embodiments, the engineered cell can comprise an induced pluripotent stem cell (iPSC). iPSCs can refer to cells derived from skin or blood cells that have been reprogrammed back into an embryonic-like pluripotent state that allows the development of an unlimited source of any type of human cell needed for therapeutic purposes. iPSCs can self-renew indefinitely in culture and differentiate into all specialized cell types. iPSCs do not exist naturally, and are instead generated (“induced” or “reprogrammed”) in culture from somatic cells through ectopic co-expression of defined pluripotency factors.

In other embodiments, the engineered cell can comprise a neural stem cell (NSC). NSCs can refer to multipotent adult stem cells present in the adult central nervous system that can self-renew and proliferate without limit, and give rise to new neurons and supporting cells called glial cells.

In still other embodiments, the engineered cell can comprise a circulatory cell. For example, the cell can comprise a hematopoietic stem cell. A hematopoietic stem cell can refer to an immature cell that can develop into all types of blood cells, including white blood cells, red blood cells, and platelets. Hematopoietic stem cells are found in the peripheral blood and the bone marrow.

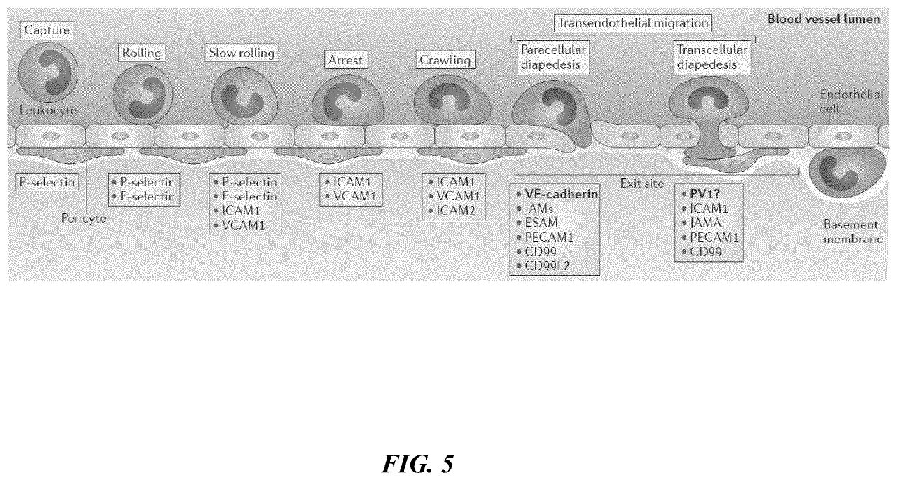

In embodiments, the engineered cell can comprise a non-circulatory cell. For example, the engineered cell can comprise a HEK 293 cell. In such embodiments, the non-circulatory cell can be engineered to behave like circulatory cells. For example, this can be accomplished through genetic engineering of the non-circulatory cell to express a non-native surface profile of proteins that are characteristic of professional circulatory cells. As exemplified in FIG. 5, homing of a professional circulatory cell to a target tissue is accomplished by combinatorial specificity of surface proteins on the circulatory cell and the target cell, such as the cell adhesion cascade that occurs to recruit leukocytes to sites of inflammation.

The cells described herein can be genetically modified or genetically engineered to express one or more proteins of interest. Genetic modification or genetic engineering can refer to a process that uses laboratory-based technologies to alter the DNA makeup of an organism. Genetic engineering techniques are known to the skilled artisan and can include the construction of DNA constructs, transient overexpression of a gene product from an exogenous transgene, integration of a transgene into the genome at random or at target loci, and/or the delivery of DNA cargo via engineered viral vectors.

In a particular embodiment, the proteins of interest can include one or more cell adhesion proteins. The term “cell adhesion protein” can refer to cell surface protein that mediates the interaction between cells, or between cells and the extracellular matrix (ECM). Non-limiting examples of cell adhesion proteins comprise PSGL-1, ESL-1, CD44, CD24, PNAd, GlyCAM, CD34, LPAM-1, JAM-A, JAMB, LFA-1, VLA-4, MAC-1, TLN1-2 (Talin), FERMT1-3 (kindlin), FUT7, FUT6, FUT4, ST3GAL6, ICAM-1, ICAM-2, LPAM-1, JAM-A, JAMB, CXCR1, CXCR2, CXCR3, CXCR4, CXCR5, CXCR6, CXCR7, CX3CR1, CCR1, CCR2, CCR3, CCR4, CCR5, CCR6, CCR7, CCR8, CCR9, CCR10, CCR11, EMR2, EMR3, CD97, MMP-1, MMP-2, MMP-9, L-selectin (SELL), GCNT1, B4GALT1, ST3GAL4, TPST1, TPST2, ITGB2, ITGAD, ITGAX, ITGAM, ITGAL, ITGB1, ITGA9, ITGA4, ITGB7, TIMP1, TIMP2, and TIMP3. In one embodiment, the cell can be genetically engineered to express PSGL-1, FUT7, and CXCR4. In another embodiment, the cell can be genetically engineered to express TSG-6, COX2, and IDO. In another embodiment, the cell can be genetically engineered to express PSGL-1, FUT7, ST3GAL4, ITGAL, ITGB2, CCR2, IL-10, CTLA4, and CD47.

In embodiments, the cell adhesion protein can comprise a P-selectin ligand, an E-selectin ligand, an L-selectin ligand, or a combination thereof. See, FIG. 5, for example, which provides combinations of cell adhesion proteins in professional circulatory cells. In one embodiment, a combination of cell adhesion proteins can comprise PSGL-1, FUT7, ST3GAL4, ITGAL, ITGB2, CCR2, IL-10, CTLA4, and CD47. For example, the P-selectin ligand can comprise PSGL-1, CD44, or CD24. For example, the E-selectin ligand can comprise PSGL-1, ESL-1, CD44, or CD24. For example, the L-selectin ligand can comprise PNAd, GlyCAM, or CD34. In embodiments, L-selectin can be expressed to engage with the L-selectin ligands of the lymph. In embodiments, a P-selectin ligand, an E-selectin ligand, an L-selectin ligand, or a combination thereof, can be expressed at a level that exceeds the level of expression of a native population of cells.

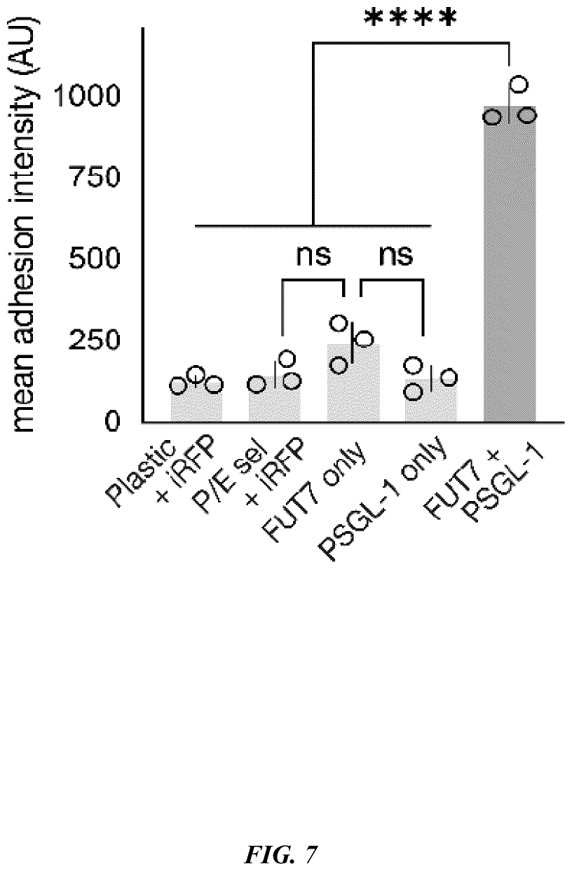

As exemplified in FIG. 6 and FIG. 7, the cell adhesion proteins described herein allows cell homing to a target tissue. The term “cell homing” can refer to the ability of circulating cells or exogenously administered cells to locate and/or enter an environmental niche. The process of homing can be split into five steps: (1) tethering and rolling, (2) activation, (3) arrest, (4) transmigration, and (5) effect (See FIG. 6).

In embodiments, expression of mediators of the signaling that occurs as a chemokine signal is converted to integrin activation can be utilized herein. For example, some mediators, such as Talin and kindlin, anchor the integrins to the cytoskeleton and allows for strong anchoring. In embodiments, mediators can be intermediate proteins that transmit the activation signals from the chemokine receptor (e.g., CXCR4) to the integrin in order to activate the integrin from a closed to open conformation. Non-limiting examples of the genes that can be utilized herein comprise GNAQ , GNB1-5, GNG1-13, GNGT1-2, PLCB1-4, RASGRP2, RAP1A&B, RASSF5, PRKCB,D,Z, CDC42, APBB1IP, ILK, YWHAZ, TIAM1, PARD3, RAC1-3, TLN1-2 (Talin), FERMT1-3 (kindlin), FLNA, DOK1, PPM1F, GNAI1-3, FGR, HCK, LYN, SRC, CDC25A, PIK3CA,B,D, PIK3R1-3, ITK, TXK, VAV1-3, and PAK1.

In embodiments, the cell can be engineered to express one or more immunomodulatory proteins. The term “immunomodulatory protein” can refer to a protein that regulates and modulates immunity. For example, the immunomodulatory protein can be in the regulation of leukocyte function. Non-limiting examples of immunomodulatory proteins comprise CCL21, CTLA4, CD39, CD73, TRAILR2, PDL1, FASL, H2-M3, IL-1R2, CD47, MFG-E8, CD200, HLA-G, HLA-E, NQO1, BCL2, Survin, or any combination thereof.

In embodiments, the cell can be engineered to express one or more survival proteins. The term “survival protein” can refer to a cellular protein that regulates apoptosis, stress resistance, or longevity by promoting cellular viability under adverse conditions. Non-limiting examples of survival proteins comprise CCL21, CTLA4, CD39, CD73, TRAILR2, PDL1, FASL, H2-M3, IL-1R2, CD47, MFG-E8, CD200, HLA-G, HLA-E, NQO1, BCL2, Survin, or any combination thereof.

In embodiments, the cell can be engineered to express a ligand for an immunomodulatory protein. For example, the ligand for an immunomodulatory protein can comprise CD47.

In embodiments, expression of immunomodulatory factors and survival genes can be tested to improve MSC persistence. Non-limiting examples of the genes that can be tested comprise CCL21, CTLA4, CD39, CD73, TRAILR2, PDL1, FASL, H2-M3, IL-1R2, CD47, MFG-E8, CD200, HLA-G, HLA-E, NQO1, BCL2, and Survin.

In embodiments, the genetically engineered cell can be engineered to express one or more safety switches. The term “safety switch” can refer to a system for controlling the expression of a gene or protein of interest that, when downregulated or upregulated, leads to clearance or death of the cell, e.g., through recognition by the host's immune system. A safety switch can be designed to be triggered by an exogenous molecule in case of an adverse clinical event. A safety switch can be engineered by regulating the expression on the DNA, RNA and protein levels. A safety switch can include a protein or molecule that allows for the control of cellular activity in response to an adverse event. In embodiments, the safety switch can be an inducible safety switch. The term “inducible safety switch” can refer to a genetically or chemically controlled mechanism designed to regulate or terminate biological activity upon activation by a specific external or internal trigger. In embodiments, the safety switch can be a small molecule inducible safety switch. In embodiments, the small molecule inducible safety switch can be deployed in the case of an immune reaction or teratoma formation. For example, the small molecule inducible safety switch can comprise iCaspase9.

In embodiments, the genetically engineered cell can be engineered to express one or more selectable markers. The term “selectable marker” can refer to a gene whose expression allows for the identification of cells that have been transformed or transfected with a vector containing the marker gene. In embodiments, the selectable marker allows for manufacturing. For example, the selectable marker can comprise puromycin acetyl transferase, truncated CD34, PSGL-1, Blasticidin, or Geneticin. In embodiments, any surface protein can be used as a selectable marker with the use of magnetic activated cell sorting (MACS) and/or fluorescence activated cell sorting (FACS).

In embodiments, the genetically engineered cell can be engineered to express one or more reporters. The term “reporter” can refer to a protein that can be easily distinguished from a background of endogenous proteins. For example, the reporter can comprise iRFP713, luciferase, eBFP2, eGFP, mCherry, or any fluorescent reporter. In embodiments, the reporter can comprise a secreted reporter. For example, a secreted reporter can comprise SEAP (secreted embryonic alkaline phosphatase).

In embodiments, the genetically engineered cell can be engineered to express at least one cargo. The terms “cargo” can refer to any protein, molecule, small molecule, or nucleic acid that is carried within the cell, such as within the vesicles of a cell's secretory system. Non-limiting examples of cargo comprises IL-4, IL-6, IL-10, IL-11, IL-35, IDO1, COX2, TSG-6, TGFB, TNFR2, IFNAR1, GDNF, BDNF, NGF, FGF1-2, NTF3, VEGF, HGF, TGFA, TRAIL, IFNB, HSF-TK, IL-12, IL-21, or IL-2.

TABLE 1
GenBank Accession Numbers and UniProt
Code for proteins contemplated herein.

| | | GenBank Accession | |
|---|---|---|---|
| Cell Adhesion Protein | | Number | UniProt Code |
| PSGL-1 | | AH003185.2 | Q14242 |
| ESL-1 | | U64791.1 | Q92896 |
| CD44 | | M24915.1 | P16070 |
| CD24 | | M58664.1 | P25063 |
| Podocalyxin (PnAd family) | | U97519.1 | O00592 |
| Endomucin (PnAd family) | | AB034695.1 | Q9ULC0 |
| GlyCAM | | AJ489589.1 | Q8IVK1 |
| CD34 | | M81104.1 | P28906 |
| L selectin | | AH003185.2 | Q14242 |
| ITGB2 | | M15395.1 | P05107 |
| ITGAD | | U37028.1 | Q13349 |
| ITGAX | | M81695.1 | P20702 |
| ITGAM | | J03925.1 | P11215 |
| PECAM | | M37780.1 | P16284 |
| ITGAL | | Y00796.1 | P20701 |
| ITGB1 | | X07979.1 | P05556 |
| ITGA9 | | D25303.1 | Q13797 |
| ITGA4 | | X16983.1 | P13612 |
| ITGB7 | | M68892.1 | P26010 |
| LFA-1 | | Combination of ITGAL & ITGB2 | |
| VLA-4 | | Combination of ITGA4 and ITGB1 | |
| MAC-1 | | Combination of ITGAM and ITGB2 | |
| Talin-1 | | AF078828.1 | Q9Y490 |
| Kindlin-3 | | AY093951.1 | Q86UX7 |
| FUT7 | | X78031.1 | Q11130 |
| FUT6 | | M98825.1 | P51993 |
| FUT4 | | M58596.1 | P22083 |
| ST3GAL4 | | L23767.1 | Q11206 |
| ST3GAL6 | | AB022918.1 | Q9Y274 |
| GCNT1 | | M97347.1 | Q02742 |
| B4GALT1 | | X14085.1 | P15291 |
| TPST1 | | AF038009.1 | O60507 |
| TPST2 | | AF061254.1 | O60704 |
| ICAM-1 | | X06990.1 | P05362 |
| ICAM-2 | | X15606.1 | P13598 |
| LPAM-1 | | Combination of ITGA4 and ITGB7 | |
| JAM-A | | AF111713.1 | Q9Y624 |
| JAMB | | AF255910.2 | P57087 |
| CXCR1 | | L19591.1 | P25024 |
| CXCR2 | | M73969.1 | P25025 |
| CXCR3 | | X95876.1 | P49682 |
| CXCR4 | | L01639.1 | P61073 |
| CXCR5 | | X68149.1 | P32302 |
| CXCR6 | | AF007545.1 | O00574 |
| CXCR7 | | M64749.1 | P25106 |
| CCR1 | | L09230.1 | P32246 |
| CCR2 | | U03882.1 | P41597 |
| CCR3 | | U28694.1 | P51677 |
| CCR4 | | X85740.1 | P51679 |
| CCR5 | | X91492.1 | P51681 |
| CCR6 | | U45984.1 | P51684 |
| CCR7 | | L08176.1 | P32248 |
| CCR8 | | Z79782.1 | P51685 |
| CCR9 | | AJ132337.1 | P51686 |
| CCR10 | | AF215981.1 | P46092 |
| CCR11 | | U97123.1 | O00421 |
| CX3CR1 | | U20350.1 | P49238 |
| EMR2 | | AF114491.1 | Q9UHX3 |
| EMR3 | | AF239764.1 | Q9BY15 |
| CD97 | | X84700.1 | P48960 |
| MMP-1 | | X54925.1 | P03956 |
| MMP-2 | | AH002654.2 | P08253 |
| MMP-9 | | J05070.1 | P14780 |
| TIMP1 | | X03124.1 | P01033 |
| TIMP2 | | J05593.1 | P16035 |
| TIMP3 | | X76227.1 | P35625 |
| | Immunomodulatory or | GenBank Accession | |
| | Survival Protein | Number | UniProt Code |
| | CCL21 | AB002409.1 | O00585 |
| | CTLA4 | L15006.1 | P16410 |
| | CD39 | S73813.1 | P49961 |
| | CD73 | X55740.1 | P21589 |
| | TRAILR2 | AF018657.1 | O14763 |
| | PDL1 | AF177937.1 | Q9NZQ7 |
| | FASL | U08137.1 | P48023 |
| | IL-1R2 | X59770.1 | P27930 |
| | CD47 | X69398.1 | Q08722 |
| | MFG-E8 | U58516.1 | Q08431 |
| | CD200 | AY603771.1 | P41217 |
| | HLA-G | X17273.1 | P17693 |
| | HLA-E | M20022.1 | P13747 |
| | NQO1 | J03934.1 | P15559 |
| | BCL2 | M13994.1 | P10415 |
| | Survivin | U75285.1 | O15392 |

| | Gen Bank Accession | |
|---|---|---|
| Selectable Marker | Number | UniProt Code |
| Puromycin acetyl | ATY35202.1 | P13249 |
| transferase | | |
| Truncated CD34 | M81104.1 (truncated) | P28906 (aa 1-131) |
| PSGL-1 | AH003185.2 | Q14242 |
| Blasticidin S deaminase | ABG81367.1 | P0C2P0 |
| Hygromycin | ACG80836.1 | P00557 |
| phosphotransferase | | |

| | GenBank Accession | |
|---|---|---|
| Cargo | Number | UniProt Code |
| IL-4 | M13982.1 | P05112 |
| IL-6 | X04430.1 | P05231 |
| IL-10 | M57627.1 | P22301 |
| IL-11 | M57765.1 | P20809 |
| IL-35 (EBI3) | L08187.1 | Q14213 |
| IL-35/IL-12 (IL-12a) | M65291.1 | P29459 |
| IL-12 (IL-12B) | M65272.1 | P29460 |
| IDO1 | M34455.1 | P14902 |
| COX2 | V00662.1 | P00403 |
| TSG-6 | M31165.1 | P98066 |
| TGFB1 | X05839.1 | P01137 |
| TGFB2 | Y00083.1 | P61812 |
| TGFB3 | J03241.1 | P10600 |
| TNFR2 | M55994.1 | P20333 |
| IFNAR1 | J03171.1 | P17181 |
| IFNAR2 | X77722.1 | P48551 |
| GDNF | AH003115.2 | P39905 |
| BDNF | M37762.1 | P23560 |
| NGF | V01511.1 | P01138 |
| FGF1 | M13361.1 | P05230 |
| FGF2 | X04431.1 | P09038 |
| NTF3 | X53655.1 | P20783 |
| VEGF | M32977.1 | P15692 |
| HGF | M29145.1 | P14210 |
| TGFA | K03222.1 | P01135 |
| TRAIL | U37518.1 | P50591 |
| IFNB | V00546.1 | P01574 |
| HSV-TK | V00470.1 | Q9QNF7 |
| IL-13 | X69079.1 | P35225 |
| IL-21 | AF254069.1 | Q9HBE4 |
| IL-2 | X00695.1 | P60568 |

| | GenBank Accession | |
|---|---|---|
| Reporter | Number | AA seq |
| iRFP713 | AEL88490.1 | See Table 2 |
| eBFP2 | ABP88743.1 | See Table 2 |
| eGFP | ANC98519.1 | See Table 2 |
| mCherry | AGD93422.1 | See Table 2 |
| Nanoluc (luciferase) | UDE22649.1 | See Table 2 |
| SEAP | QIM58251.1 | See Table 2 |

TABLE 2
Amino acid sequences of reporters described herein.

| | |
|---|---|
| iRFP713 | MAAEGSVARQPDLLTCDDEPIHIPGAIQPHGLLLALAA |
| | DMTIVAGSDNLPELTGLAIGALIGRSAADVFDSETHNR |
| | LTIALAEPGAAVGAPITVGFTMRKDAGFIGSWHRHDQ |
| | LIFLELEPPQRDVAEPQAFFRRTNSAIRRLQAAETLESA |
| | CAAAAQEVRKITGFDRVMIYRFASDFSGEVIAEDRCA |
| | EVESKLGLHYPASTVPAQARRLYTINPVRIIPDINYRPV |
| | PVTPDLNPVTGRPIDLSFAILRSVSPVHLEFMRNIGMH |
| | GTMSISILRGERLWGLIVCHHRTPYYVDLDGRQACEL |
| | VAQVLAWQIGVMEE* (SEQ ID NO: 1) |
| eBFP2 | MAVSKGEELFTGVVPILVELDGDVNGHKFSVRGEGEG |
| | DATNGKLTLKFICTTGKLPVPWPTLVTTLSHGVQCFA |
| | RYPDHMKQHDFFKSAMPEGYVQERTIFFKDDGTYKT |
| | RAEVKFEGDTLVNRIELKGVDFKEDGNILGHKLEYNF |
| | NSHNIYIMAVKQKNGIKVNFKIRHNVEDGSVQLADHY |
| | QQNTPIGDGPVLLPDSHYLSTQSVLSKDPNEKRDHMV |
| | LLEFRTAAGITLGMDELYK* (SEQ ID NO: 2) |
| eGFP | MAMVSKGEELFTGVVPILVELDGDVNGHKFSVSGEGE |
| | GDATYGKLTLKFICTTGKLPVPWPTLVTTLTYGVQCF |
| | SRYPDHMKQHDFFKSAMPEGYVQERTIFFKDDGNYK |
| | TRAEVKFEGDTLVNRIELKGIDFKEDGNILGHKLEYNY |
| | NSHNVYIMADKQKNGIKVNFKIRHNIEDGSVQLADHY |
| | QQNTPIGDGPVLLPDNHYLSTQSALSKDPNEKRDHMV |
| | LLEFVTAAGITLGMDELYK* (SEQ ID NO: 3) |
| mCherry | MAGNSVSKGEEDNMAIIKEFMRFKVHMEGSVNGHEF |
| | EIEGEGEGRPYEGTQTAKLKVTKGGPLPFAWDILSPQF |
| | MYGSKAYVKHPADIPDYLKLSFPEGFKWERVMNFED |
| | GGVVTVTQDSSLQDGEFIYKVKLRGTNFPSDGPVMQK |
| | KTMGWQASSERMYPEDGALKGEIKQRLKLKDGGHY |
| | DAEVKTTYKAKKPVQLPGAYNVNIKLDITSHNEDYTI |
| | VEQYERAEGRHSTGGMDELYK* (SEQ ID NO: 4) |
| Nanoluc | MVFTLEDFVGDWRQTAGYNLDQVLEQGGVSSLFQNL |
| (luci- | GVSVTPIQRIVLSGENGLKIDIHVIIPYEGLSGDQMGQIE |
| ferase) | KIFKVVYPVDDHHFKVILHYGTLVIDGVTPNMIDYFG |
| | RPYEGIAVFDGKKITVTGTLWNGNKIIDERLINPDGSLL |
| | FRVTINGVTGWRLCERILA* (SEQ ID NO: 5) |
| SEAP | MVLGPCMLLLLLLLGLRLQLSLGIIPVEEENPDFWNRE |
| | AAEALGAAKKLQPAQTAAKNLIIFLGDGMGVSTVTA |
| | ARILKGQKKDKLGPEIPLAMDRFPYVALSKTYNVDKH |
| | VPDSGATATAYLCGVKGNFQTIGLSAAARFNQCNTTR |
| | GNEVISVMNRAKKAGKSVGVVTTTRVQHASPAGTYA |
| | HTVNRNWYSDADVPASARQEGCQDIATQLISNMDIDV |
| | ILGGGRKYMFRMGTPDPEYPDDYSQGGTRLDGKNLV |
| | QEWLAKRQGARYVWNRTELMQASLDPSVTHLMGLF |
| | EPGDMKYEIHRDSTLDPSLMEMTEAALRLLSRNPRGF |
| | FLFVEGGRIDHGHHESRAYRALTETIMEDDAIERAGQL |
| | TSEEDTLSLVTADHSHVFSFGGYPLRGSSIFGLAPGKA |
| | RDRKAYTVLLYGNGPGYVLKDGARPDVTESESGSPEY |
| | RQQSAVPLDEETHAGEDVAVFARGPQAHLVHGVQEQ |
| | TFIAHVMAFAACLEPYTACDLAPPAGTTDAAHPGRSR |
| | SKRLD* (SEQ ID NO: 6) |

In embodiments, the cargo can comprise a vector. A “vector” can refer to a tool that allows or facilitates the transfer of an entity from one environment to another. In particular embodiments, the vector can be a transposon-based vector. A “transposon” can refer to a DNA segment which is able to change its relative position within the entire genome of a cell. For example, the transposon-based vector can be a piggyBac transposon vector. The “piggyBac (PB) transposon” can refer to a movable genetic element that efficiently transposes between vectors and chromosomes through a “cut-and-paste” mechanism.

In embodiments, the vector can comprise one or more expression units (EU). In embodiments, an expression unit can comprise transcriptional/translational regulatory elements. In embodiments, the transcriptional/translational regulatory elements can comprise a promoter and/or a terminator, and an open reading frame. In embodiments, the open reading frame can encode a protein such as, but not limited to, a secreted therapeutic, surface modification, regulatory genetic circuit, or reporter. In embodiments, the expression unit can express IL-2, IL-4, IL-6, IL-10, IL-11, IL-12, IL-21, IL-35, IDO1, COX2, TSG-6, TGFB, TNFR2, IFNAR1, PD-L1, PD-L2, CD40, CTLA4, GDNF, BDNF, NGF, FGF1-2, NTF3, VEGF, HGF, TGFa, TRAIL, IFNB, and HSV-TK.

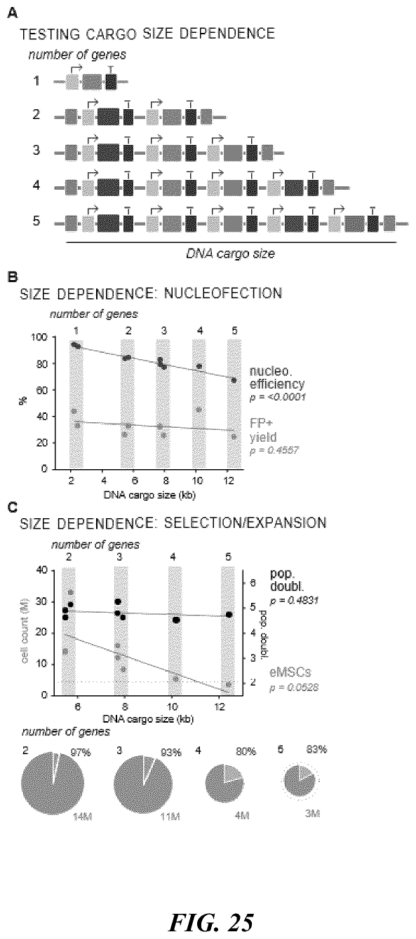

In embodiments, the cargo described herein can be expressed from a multi-transgene (i.e., multi-transgene cargo). The term multi-transgene can refer to a nucleic acid construct, such as a vector, comprising multiple expression units. See, for example, FIG. 25.

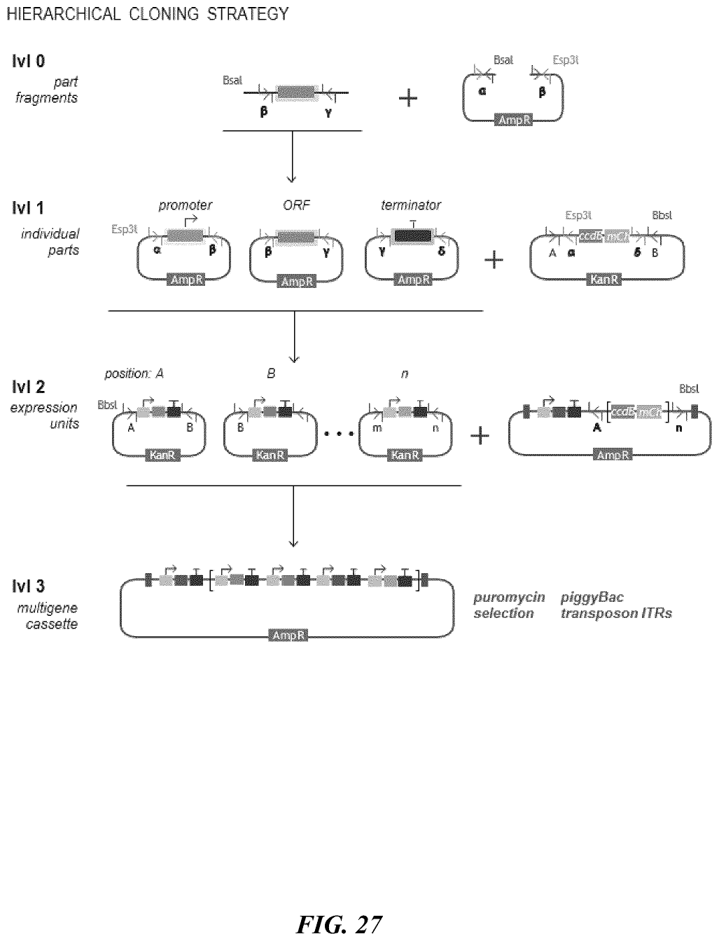

Referring to FIG. 27, for example, multigene cassettes can be constructed by assembling one or more expression units into nucleic acid vectors. Examples as described herein utilize a custom piggyBac transposon destination vector; however, the skilled artisan will recognize that any nucleic acid vector that is capable of harboring one or more expression units can be used. An “expression cassette” can refer to a distinct component of vector DNA consisting of a gene and regulatory sequence to be expressed by a transfected cell. In each successful transformation, the expression cassette directs the cell's machinery to make RNA and protein(s). Expression cassettes can comprise a promoter, the gene-of interest (open reading frame, ORF), and a terminator. Expression cassettes can be introduced into a vector that facilitates entry of the expression cassette into a host cell and maintenance of the expression cassette in the host cell. Such vectors are commonly used and are well known to those of skill in the art. In embodiments, the vector can comprise a custom piggyBac transposon destination vector. For example, an expression cassette of the invention can comprise puromycin acetyl transferase, PSGL-1, FUT7 (+/−ST3GAL4), ITGAL, ITGB2, CCR2 (or a different chemokine receptor), CD47 (+/−HLA-G), iCasp9 safety switch, IL-10 as a therapeutic cargo, iRFP713 reporter, or any combination thereof. For example, an expression cassette of the invention can comprise puromycin acetyl transferase, PSGL-1, FUT7 (+/−ST3GAL4), ITGaL, ITGB2, CCR2 (or a different chemokine receptor), or any combination thereof. For example, an expression cassette of the invention can comprise Blasticidin, CD47 for immune cloaking (+/−HLA-G), iCasp9 safety switch, IL-10 as a therapeutic cargo (+others), or any combination thereof.

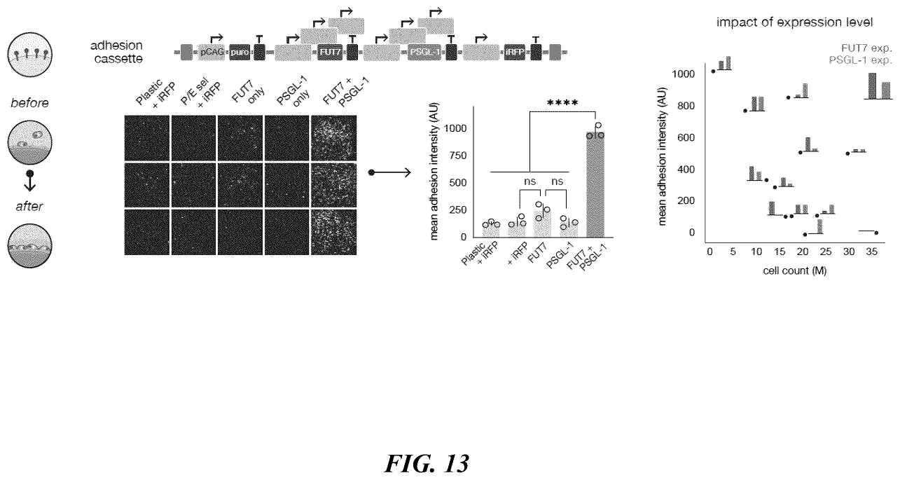

The cargo can be re-configured to generate products with fine-tuned nonnative function, such as fine-tuned nonnative immunomodulatory function. For example, the cargo can be re-configured through expression level through promoter/Kozak selection and/or modulation of the therapeutic cargo. As exemplified in FIG. 13 for example, the independent tunability of each gene in the multi-transgene cargo allows for the optimization for a) expression, b) function, and c) yield of the cell product. For example, medium expression of PSGL-1 and low expression of FUT7 provides an ideal combination of expression, function, and yield of the cell product for adhesion to P/E selectin in vitro and inflammation targeting in vivo. Expression level can be important because high expression of both factors can be toxic and can't be used to produce a cell product that meets clinical scale.

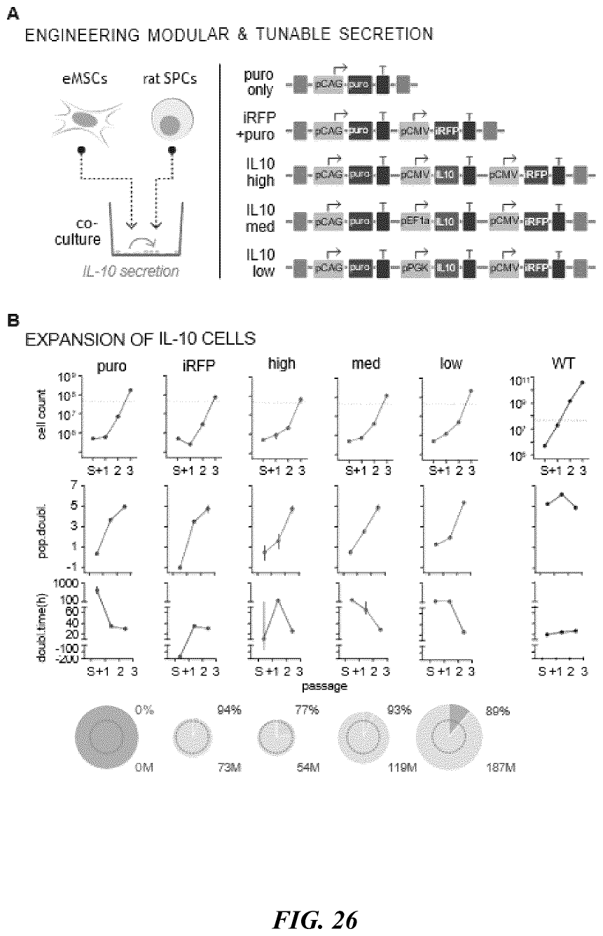

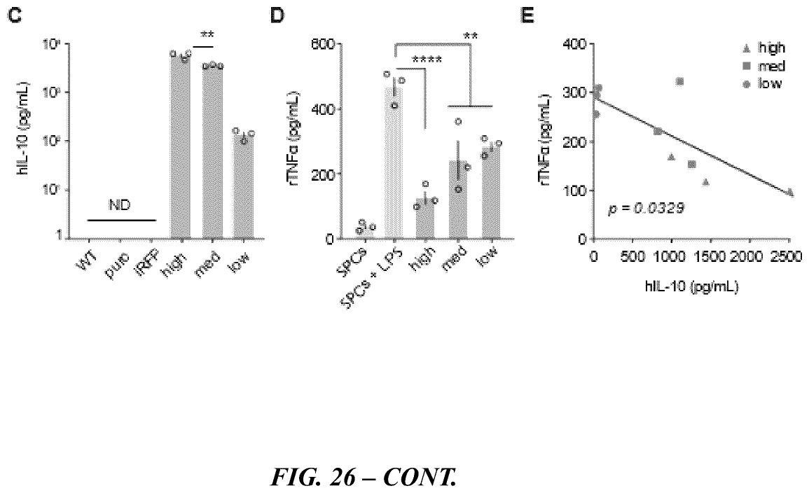

In embodiments, the multi-transgene cargo comprises multiple expression units (EU) (such as two EUs, three EUs, four EUs, five EUs, six EUs, seven EUs or greater than seven EUs), each unit having independently tunable protein expression (See FIG. 26, for example). In embodiments, promoter selection and Kozak sequence can provide the tunable protein expression described herein. In embodiments, protein expression can be tuned via dose response.

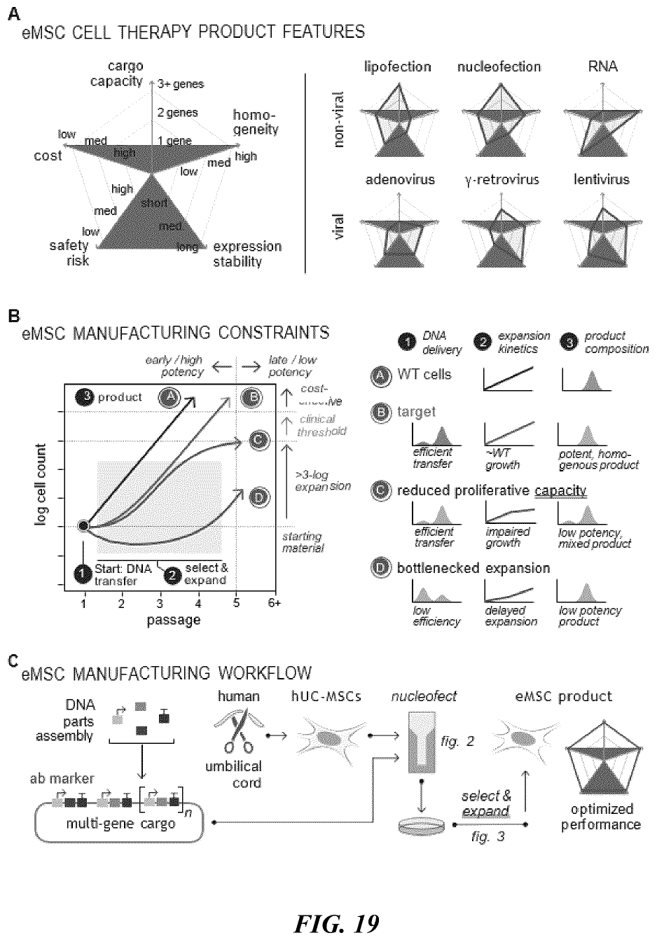

Aspects of the invention are also drawn to a method of manufacturing the genetically engineered cell(s) described herein. As exemplified in FIG. 19, the method described herein provides a manufacturing strategy that supports clinical translation of eMSCs. A longstanding problem in the field is manufacturing sufficient quantities of genetically modified cells, such as MSCs, that maintain potency and can therefore be used for clinical trials. See, for example, PMID: 27745603, PMID: 32832666, PMID: 32832666. For example, the method described herein provides for manufacturing sufficient quantities of genetically modified MSCs that maintain potency. Without wishing to be bound by theory, the method will yield a cell product with one or more of the following features: 1) the DNA cargo harbored by the cells should be large enough to encode multiple transgenes; 2) transgene expression should be homogenous to ensure functional similarity across the cell population; 3) expression should show long-term stability; 4) genetic manipulation and expansion should not impact the product safety profile; and/or 5) the overall cost of generating the product from starting materials should be minimized.

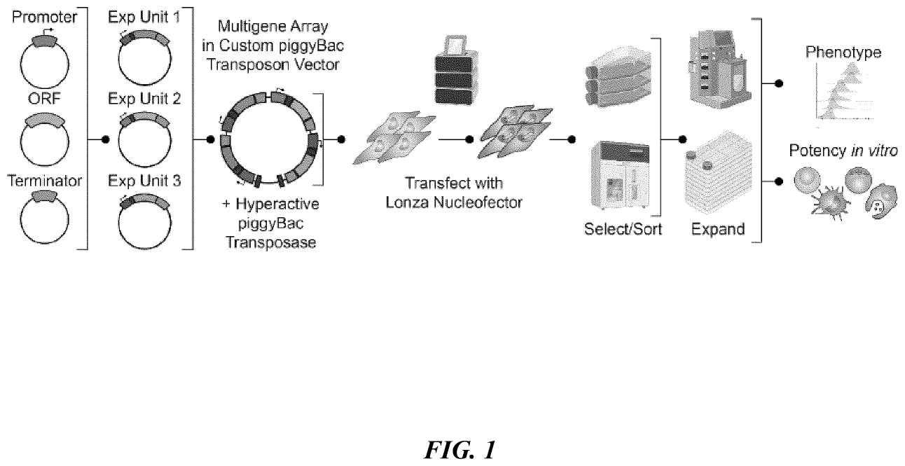

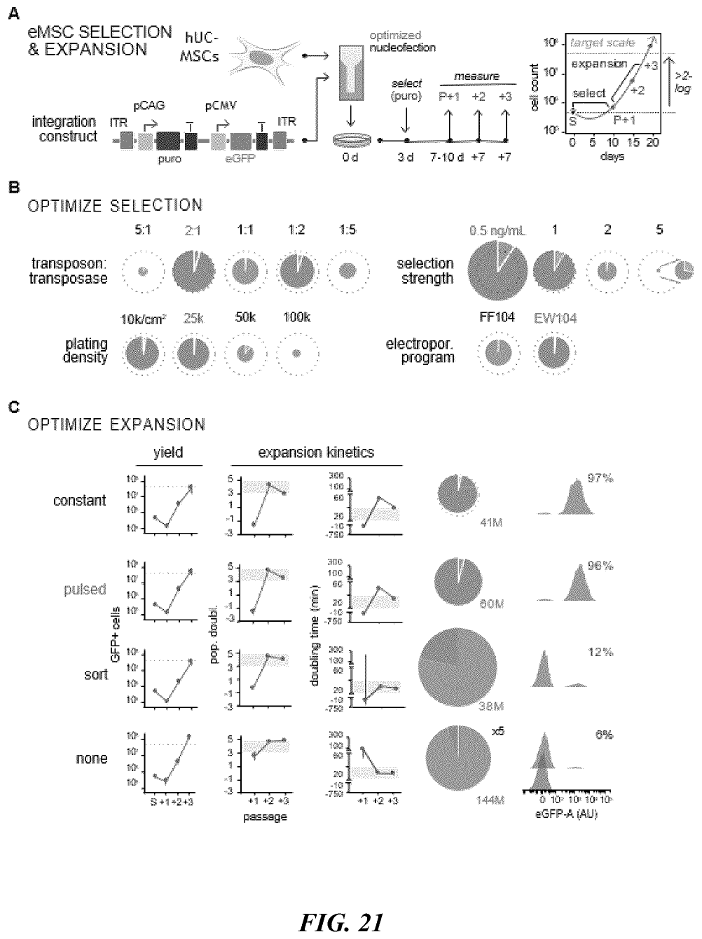

As exemplified in FIG. 1, this method comprises isolating and harvesting a source cell or population of source cells, such as a population of MSCs, expanding the isolated cells, delivering (e.g., transient transfection and/or integration/stable expression) the DNA cargo to the cells (thereby providing engineered cells), selecting for the engineered cells, and expanding the engineered cells to a clinically relevant dose. In embodiments, the cell can be harvested from a subject or a biological sample. In embodiments, the method further comprises selecting for the cells that express the proteins. In embodiments, the isolated cells and/or the genetically engineered cells described herein can be expanded (See FIG. 21, for example).

The term “isolating” or “isolating a cell” can refer to the process of separating a specific cell from a heterogeneous population using physical, chemical, or biological techniques to obtain a purified or enriched cell population suitable for analysis or therapeutic use.

The term “harvesting” or “harvesting a cell” can refer to the controlled process of collecting biological, chemical, or physical materials from a source after a growth, production, or synthesis phase, ensuring preservation and functionality for further use.

Aspects of the invention are drawn to a method of treating, preventing, or ameliorating the symptoms of a disease, disorder or medical condition manifesting as inflamed and/or damaged tissue in a subject. As used herein, the terms “treat” or “treatment” refer to both therapeutic treatment and prophylactic or preventative measures, wherein the object is to prevent or slow down (lessen) an undesired physiological change or disorder, such as the progression of a disease, disorder or medical condition manifesting as inflamed and/or damaged tissue in a subject. Beneficial clinical results include, but are not limited to, alleviation of symptoms, diminishment of extent of disease, stabilized (i.e., not worsening) state of disease, delay or slowing of disease progression, amelioration or palliation of the disease state, and remission (whether partial or total), whether detectable or undetectable. “Treatment” can refer to prolonging survival as compared to expected survival if not receiving treatment. Those in need of treatment include those already with the condition or disorder as well as those prone to have the condition or disorder or those in which the condition or disorder is to be prevented.

Herein, “preventing” a disease can refer to inhibiting the full development of a disease, such as a disease, disorder or medical condition manifesting as inflamed and/or damaged tissue in a subject. “Treating” can refer to a therapeutic intervention that ameliorates a sign or symptom of a disease or pathological condition after it has begun to develop. “Ameliorating” can refer to the reduction in the number or severity of signs or symptoms of a disease, disorder or medical condition manifesting as inflamed and/or damaged tissue in a subject. A “prophylactic” treatment is a treatment administered to a subject who does not exhibit signs of a disease or exhibits only early signs for the purpose of decreasing the risk of developing a pathology.

“Inflammation” can refer to is the immune system's response to harmful stimuli, such as pathogens, damaged cells, toxic compounds, or irradiation, and acts by removing injurious stimuli and initiating the healing process. Usually, during acute inflammatory responses, cellular and molecular events and interactions efficiently minimize impending injury or infection. This mitigation process contributes to restoration of tissue homeostasis and resolution of the acute inflammation. However, uncontrolled acute inflammation may become chronic, contributing to a variety of chronic inflammatory diseases. In embodiments, inflammation can be acute or chronic. For example, inflammation can be tissue inflammation or systemic inflammation. For example, inflammation can be neuroinflammation.

“Neuroinflammation” can refer to an inflammatory response within the brain or spinal cord. This inflammation is mediated by the production of cytokines, chemokines, reactive oxygen species, and secondary messengers.

“Systemic inflammation” can refer to an inflammatory response throughout the entire body.

In embodiments, the disease, disorder or medical condition comprises neuroinflammation. For example, the disease, disorder or medical condition comprises traumatic brain injury. The terms “traumatic brain injury” and “TBI” can refer to can refer to a disruption in normal brain function caused by an external force, which may result in neurological, cognitive, or physiological impairments. Such injury may be characterized by vascular damage, neuronal degeneration, metabolic dysfunction, or inflammatory responses, and may be classified by severity based on objective clinical or imaging criteria. Primary injury can cause irreversible tissue damage. Secondary injury from brain inflammation can cause further damage and can lead to disability.

In other embodiments, the disease, disorder or medical comprises other diseases characterized by inflammation, including stroke, acute graft vs. host disease, inflammatory bowel disease (e.g., Crohn's disease and/or ulcerative colitis and associated complications), autoimmune diseases, cancer, or any combination thereof.

As exemplified in FIG. 6, the genetic engineering described herein allows targeting and ameliorating inflammation of a target tissue. For example, the target tissue can comprise neuronal tissue.

In embodiments, the method comprises administering to the subject a population of the genetically engineered cells described herein. Methods of administration of the cells to a subject are within the knowledge of the skilled artisan and can include infusion of the cells and/or injection of the cells into a target tissue. Herein, “administration” can refer to the act of the attending physician or caregiver, prescribing the agent for administration and thereby causing the application of an agent to a subject, through ingestion, infusion, injection, or any other means, whether self-administered or administered by a clinician or other qualified care giver. For example, administration of the engineered cell disclosed herein can abrogate or inhibit or interfere with an activity of the target protein(s).

The cells may be allogeneic and/or autologous with respect to the subject to be treated. The term “allogenic” or “allogenic stem cells” can refer to cells collected from a donor. The term “autologous” or “autologous stem cells” can refer to cells collected from the subject. In some embodiments, the cells are derived from blood, bone marrow, adipose tissue, umbilical cord tissue, lymph or lymphoid organs, or cells of the immune system.

In embodiments, the engineered cell is an autologous MSC. For example, the autologous MSC can comprise a bone marrow-derived MSC or an adipose tissue-derived MSC. In further embodiments, the MSC is an allogenic MSC. For example, the allogenic MSC can comprise an umbilical cord-derived MSC, an adipose-derived tissue-derived MSC, or a bone marrow-derived MSC.

Herein, a “subject” includes both human patient and veterinary subjects, including human and non-human mammals. In embodiments, the subject or patient has or has a risk of cancer. Mammals include, but are not limited to, domesticated animals (e.g., cows, sheep, cats, dogs, and horses), primates (e.g., humans and non-human primates such as monkeys), rabbits, and rodents (e.g., mice and rats). In certain embodiments, the subject is a human.

### OTHER EMBODIMENTS

While the invention has been described in conjunction with the detailed description thereof, the foregoing description is intended to illustrate and not limit the scope of the invention, which is defined by the scope of the appended claims. Other aspects, advantages, and modifications are within the scope of the following claims.

The invention will be further described in the following examples, which do not limit the scope of the invention described in the claims.

### EXAMPLES

Examples are provided below to facilitate a more complete understanding of the invention. The following examples illustrate the exemplary modes of making and practicing the invention. However, the scope of the invention is not limited to specific embodiments disclosed in these Examples, which are for purposes of illustration only, since alternative methods can be utilized to obtain similar results.

### Example 1

### Clinically Compatible Method to Genetically Engineer MSCs

This invention is a new process through 1 which MSCs (mesenchymal/multipotent/medicinal stem/stromal/signaling cell/cells) can be genetically engineered with user-defined programs. These programs can be comprised of an array of multiple expression units (1-5+) that each have independently tunable protein expression. An expression unit includes transcriptional/translational regulatory elements including but not limited to a promoter and/or terminator and an open reading frame encoding a protein such as, but not limited to, a secreted therapeutic, surface modification, regulatory genetic circuit, or reporter. This process takes MSCs sourced from autologous or allogeneic tissue (e.g., adipose, bone marrow, or umbilical cord) via clinically compatible methods, delivers the DNA cargo, optionally selects for the engineered cells, then expands those cells to a clinically relevant dose.

Without wishing to be bound by theory, mesenchymal stem/stromal cells (MSCs) can be a chassis for cell-based therapy due to their inherent immune modulatory and regenerative properties^{1,2}, immune evasive phenotype³, and manufacturability from minimally invasive tissue sources⁴. Without wishing to be bound by theory, the ability to augment natural MSC function by introducing genetic programs that custom-define the properties of the cell product can overcome clinical barriers, unlocking MSCs to be utilized as potent therapeutic agents across multiple indications. However, attempts to produce genetically engineered MSC products have yet to show widespread clinical efficacy due to challenges with DNA delivery and product expansion^{5,6}. This method presents a new cell engineering approach that can generate clinically compatible MSC products programmed with complex, multi-gene programs that enhance therapeutic potency.

Key Aspects for this Method Include (See FIG. 1):
Cell choice and preparation
Optimized cell engineering
A customized transposon-based vector for multi-gene DNA delivery

This process begins by sourcing MSCs that can proliferate to a clinical dose from either autologous (e.g., adipose, bone marrow) or allogeneic (e.g., adipose, bone marrow, umbilical cord) sources. While this process can be adapted to MSCs from any tissue source, exemplary sources are adipose tissue and umbilical cords. P0 MSCs are isolated according to established protocols⁷⁻¹³. These cells are then expanded for one passage (P1) according to GMP-compatible methods in xeno-free media (aMEM, human platelet lysate, Glutamax, gentamycin) on tissue culture treated plastic.

P1 MSCs are then engineered by delivering a customized transposon-based vector using the manufacturing process summarized herein. This transposon is comprised of i) a selectable marker such as an antibiotic resistance marker (e.g., puromycin acetyl transferase) or a surface marker (e.g., truncated CD34), ii) an array of functional expression units, where each expression unit includes transcriptional/translational regulatory elements including but not limited to a promoter and/or terminator and an open reading frame encoding a protein such as, but not limited to, a secreted therapeutic, surface modification, or regulatory genetic circuit, and iii) an optional reporter expression unit (e.g., fluorescent reporter such as iRFP713 or eGFP, bioluminescent reporter such as luciferase, or secreted factor such as secreted embryonic alkaline phosphatase (SEAP)).

### Summary of Manufacturing Process:

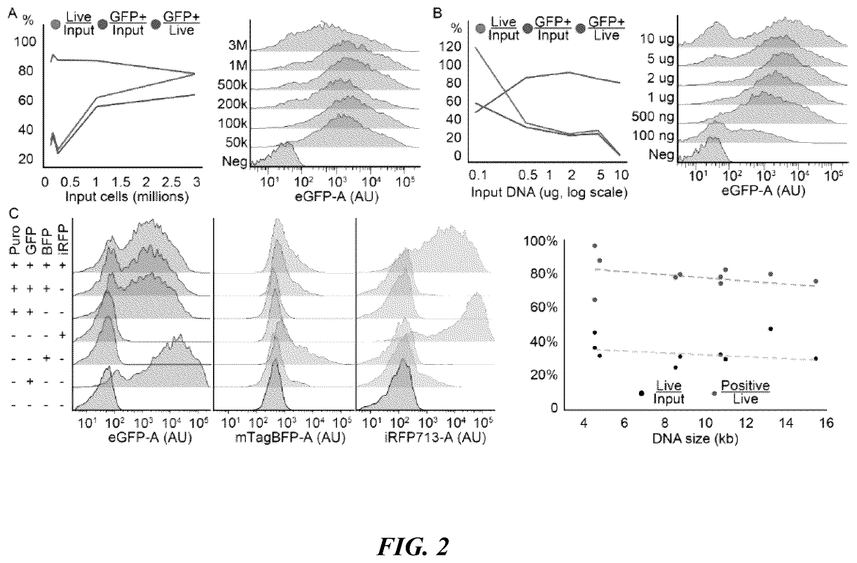

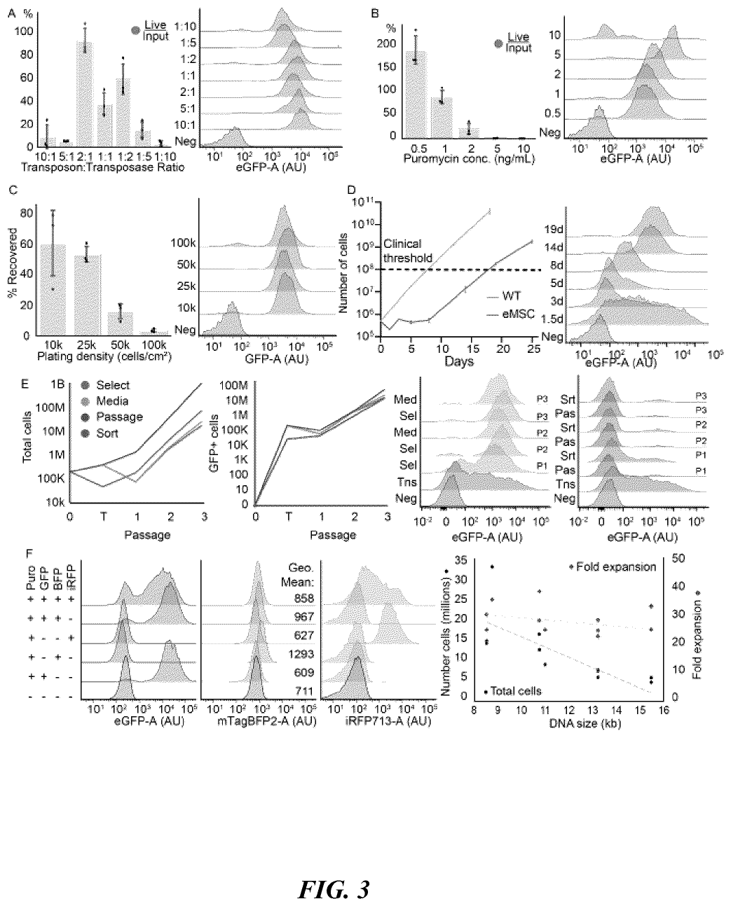

1. Isolate and harvest P0 MSCs using clinically established protocols
2. Expand MSCs one passage to P1
3. Deliver DNA to the P1 MSCs and select for stable expression
   a. Use Lonza nucleofector (e.g., 2b, 4D) without modification
   b. Use Lonza proprietary buffer (P1)
   c. Use Lonza proprietary electroporation program (e.g., EW104, although Lonza recommends FF104)
   d. Use increased amount of cells/reaction (Optimum 500k for small cuvette, range 500k to 3M, Lonza recommended 50k) (See FIG. 2A)
      i. This can be scaled for larger cuvettes and nucleofection formats
   e. Use increased amount of DNA/reaction (Optimum 1-2 ug, range 500 ng to 5 ug, Lonza recommended 200 ng to 1 ug) (See FIG. 2B)
      i. This can be scaled for larger cuvettes and nucleofection formats f. Use a 2:1 transposon:tranposase ratio (Manufacturer recommends anywhere between 5:1, 2.5:1, or 1:1) and use hyperactive piggyBac transposase (See FIG. 3A)
   g. Use a gentle antibiotic selection (e.g., puromycin at 0.5 ng/ml) or a cell sorting using a genetically encoded surface marker (e.g., truncated CD34) via magnetic activated cell sorting (MACS) or a fluorescence reporter via fluorescence activated cell sorting (FACS). (See FIG. 3B)
   h. Use a reduced plating density (Optimum 10k cells/cm2, range 10k-50k, Lonza recommended 50k) (See FIG. 3C)
4. Expand the engineered MSCs to a clinical dose in tissue culture plastic (e.g., T225s, Cell Factory, 3D bioreactors). For 2D culture, cells are plated at 1k-3k cells/cm2 and passaged at ˜90% confluency. For rapidly expanding cells, 3k cell/cm2 are plated (i.e., 10M cells on a 5-layer cell stack), media is changed at Day 3, and cells are harvested at Day 6. (See FIG. 3D-E)
   a. Example expansion: 1M POs à 50M Pls à 400M eP2s à 4B eP3s à 60B eP4s à 1T eP5s (Assumed dose=1B/patient, therefore 1000 doses/1M POs)
5. Modifications may be required to accommodate large multi-gene arrays. We found DNA size had no impact on transfection efficiency or transient yield (See FIG. 2C) but increasing DNA size reduced the yield when integrated (See FIG. 3F). However, the resultant cells have the same expansion potential as smaller genetic constructs. Therefore, more cells may be required as input with a higher cell/cm2 plating density.

This protocol can be adapted by using later passage MSCs for applications that do not require full clinical scale expansion. Additionally, a Master Cell Bank would be established at P2, and a Working Cell Bank at P3. This method could be further modified by using other integration methods that may result in higher integration efficiencies, such as a recently described version of a piggyBac transposase¹⁴ or alternative integration strategy (e.g., PASTE¹⁵).

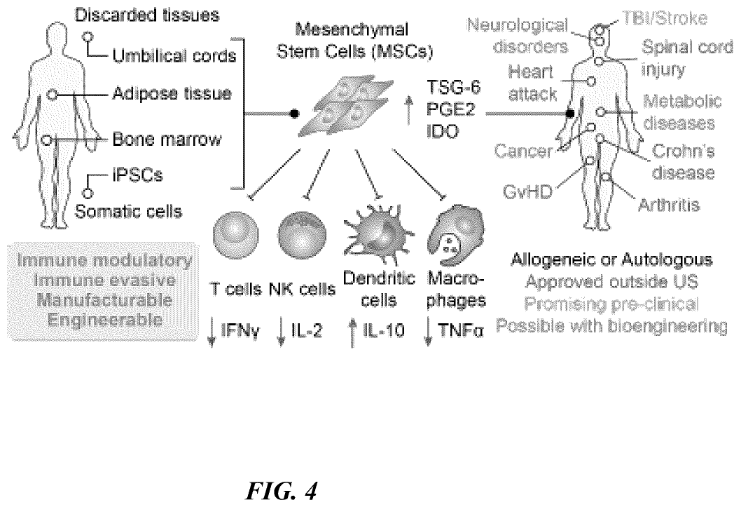

Mesenchymal stem/stromal cells (MSCs) have been extensively studied as allogeneic cell therapies across diverse indications¹ due to their inherent immunomodulatory and regenerative properties², their immune evasive phenotype³, and their manufacturability from minimally invasive or discarded tissue sources⁴ (See FIG. 4). Despite this potential and promising pre-clinical studies, MSC-based therapies consistently fail in clinical trials across multiple indications⁶. Importantly, the overwhelming consensus from these trials is that MSC infusion (both autologous and allogeneic) is safe and well tolerated by patients, although there is little clinical benefit¹. While these are significant challenges for traditional MSC cell therapy, we could view MSCs instead as an engineerable chassis. If it were possible to enhance this chassis with augmented potency, it could be safely deployed as an allogeneic therapy for a diverse array of indications.

Efforts to genetically engineer MSCs have led to hundreds of proof-of-concept and pre-clinical studies¹⁶, a few clinical trials¹⁷, but no FDA approved products¹. Both non-viral⁵ and viral¹⁸ methods have been deployed in MSCs, with advantages and disadvantages to each strategy. The most clinically successful genetically engineering MSC product to date uses a non-viral, transient transfection with a chemical transfection agent (Fugene)^{17,19-22}. However, this approach has been shown to produce a heterogeneous product, with transfection efficiencies ranging from 5 to 75% ^{5,23} and a detrimental impact to the immunomodulatory properties²⁴ and viability⁵ of the MSCs. Electroporation and nucleofection are alternative non-viral strategies that have shown transfection efficiencies>90% with reduced toxicity²⁵. Both methods have high DNA cargo capacity (>15 kb) for short term, transient expression (˜72 hr) or stable expression when combined with transposonbased²⁶ or CRISPR-based²⁷ integration. Lentivirus, on the other hand, has a high transduction efficiency ranging from 50-70%^{18,28} and preserves proliferation and differentiation capabilities¹⁸.

This method has a moderate cargo capacity of ˜8 kb, has a demonstrated clinical safety record²⁹, and produces cells with stable expression of the transgene. While both viral and non-viral methods have been sufficient for studies with simple overexpression of one or two genes¹⁶, an ideal therapeutic program would be comprised of multiple genes that are independently tunable (e.g., enhancing localization and potency simultaneously), requiring DNA loading capacity beyond that of lentivirus and clinical compatibility beyond current non-viral approaches.

This process produces genetically engineered MSCs capable of clinically compatible expansion without resorting to “reversible and conditional immortalization” to overcome the “rapidly decreased growth kinetics during in vitro expansion” that others have reported³⁰. Therefore, without wishing to be bound by theory, these primary MSCs will exhibit the same safety profiles as their unmodified counterparts³. This process also exhibits larger DNA cargo capacity than that afforded by lentiviral transduction¹⁸, while simultaneously improving the safety profile of the product⁵. Together, this allows MSCs to be engineered with multiple genes (current engineering strategies cap at two genes^{1,16,31}) and expand to clinical doses. Without wishing to be bound by theory, this work will result in a generational advance for allogeneic cell therapies by developing eMSCs as a broadly generalizable chassis that overcomes current clinical barriers that can be augmented with user-defined and indication-specific features.

Embodiments may utilize the piggyBac transposase, other transposases (e.g., newly described quantum piggyBac transposase¹⁴), or different integration methods (e.g., newly described PASTE system¹⁵). Without wishing to be bound by theory, the steps in the process would be similar, although the plating density and transposon: transposase ratio will change. Additionally, the transposase-based integration method inherently integrates randomly both in number and location. In embodiments, a CRISPR based integration strategy allows for single copy integration in a defined genetic locus and could result in a more homogenous product. In embodiments, the modifications and processes used in this method can be FDA approved and/or GMP compatible.

### REFERENCES CITED IN THIS EXAMPLE

1. Levy, O. et al. Shattering barriers toward clinically meaningful MSC therapies. Sci Adv 6, 1-19 (2020).
2. Pittenger, M. F. et al. Mesenchymal stem cell perspective: cell biology to clinical progress. NPJ Regen Med 4, (2019).
3. Hare, J. M. et al. Comparison of allogeneic vs autologous bone marrow-derived mesenchymal stem cells delivered by transendocardial injection in patients with ischemic cardiomyopathy: The POSEIDON randomized trial. JAMA—Journal of the American Medical Association 308, 2369-2379 (2012).
4. Yin, J. Q., Zhu, J. & Ankrum, J. A. Manufacturing of primed mesenchymal stromal cells for therapy. Nat Biomed Eng 3, 90-104 (2019).
5. Hamann, A., Nguyen, A. & Pannier, A. K. Nucleic acid delivery to mesenchymal stem cells: A review of nonviral methods and applications. J Biol Eng 13, 1-16 (2019).
6. Galipeau, J. & Sensébé, L. Mesenchymal Stromal Cells: Clinical Challenges and Therapeutic Opportunities. Cell Stem Cell 22, 824-833 (2018).
7. Skiles, M. L., Brown, K. S., Tatz, W., Swingle, K. & Brown, H. L. Quantitative analysis of composite umbilical cord tissue health using a standardized explant approach and an assay of metabolic activity. Cytotherapy 20, 564-575 (2018).
8. Skiles, M. L., Marzan, A. J., Brown, K. S. & Shamonki, J. M. Comparison of umbilical cord tissuederived mesenchymal stromal cells isolated from cryopreserved material and extracted by explantation and digestion methods utilizing a split manufacturing model. Cytotherapy 22, 581-591 (2020).
9. Engelhardt, M. et al. Progenitor cell expansion Hematopoietic recovery of ex vivo perfusion culture expanded bone marrow and unexpanded peripheral blood progenitors after myeloablative chemotherapy. Bone Marrow Transplant 27, 249-259 (2001).
10. Fang, B., Song, Y., Liao, L., Zhang, Y. & Zhao, R. C. Favorable Response to Human Adipose Tissue-Derived Mesenchymal Stem Cells in Steroid-Refractory Acute Graft-Versus-Host Disease. Transplant Proc 39, 3358-3362 (2007).
11. Krampera, M., Galipeau, J., Shi, Y., Tarte, K. & Sensebe, L. Immunological characterization of multipotent mesenchymal stromal cells—The international society for cellular therapy (ISCT) working proposal. Cytotherapy 15, 1054-1061 (2013).
12. Dominici, M. et al. Minimal criteria for defining multipotent mesenchymal stromal cells. The International Society for Cellular Therapy position statement. Cytotherapy 8, 315-317 (2006).
13. Majore, I., Moretti, P., Stahl, F., Hass, R. & Kasper, C. Growth and Differentiation Properties of Mesenchymal Stromal Cell Populations Derived from Whole Human Umbilical Cord. Stem Cell Reviews and Reports 2010 7:1 7, 17-31 (2010).
14. Hua, W.-K. et al. Quantum pBac: An effective, high-capacity piggyBac-based gene integration vector system for unlocking gene therapy potential. bioRxiv 2022.04.29.490002 (2022) doi:10.1101/2022.04.29.490002.
15. Yarnall, M. T. N. et al. Drag-and-drop genome insertion of large sequences without double-strand DNA cleavage using CRISPR-directed integrases. Nature Biotechnology 2022 1-13 (2022) doi:10.1038/s41587-022-01527-4.
16. Damasceno, P. K. F. et al. Genetic Engineering as a Strategy to Improve the Therapeutic Efficacy of Mesenchymal Stem/Stromal Cells in Regenerative Medicine. Front Cell Dev Biol 8, 1-24 (2020).
17. Kawabori, M. et al. Cell Therapy for Chronic TBI. Neurology 96, e1202-e1214 (2021).
18. Lin, P. et al. Efficient lentiviral transduction of human mesenchymal stem cells that preserves proliferation and differentiation capabilities. Stem Cells Transl Med 886-897 (2012) doi: http://dx.doi.org/10.5966/sctm.2012-0086.
19. Dezawa, M. et al. Specific induction of neuronal cells from bone marrow stromal cells and application for autologous transplantation. J Clin Invest 113, 1701-1710 (2004).
20. Tate, C. C., Fonck, C., McGrogan, M. & Case, C. C. Human mesenchymal stromal cells and their derivative, SB623 cells, rescue neural cells via trophic support following in vitro ischemia. Cell Transplant 19, 973-984 (2010).
21. Yasuhara, T. et al. Notch-Induced Rat and Human Bone Marrow Stromal Cell Grafts Reduce Ischemic Cell Loss and Ameliorate Behavioral Deficits in Chronic Stroke Animals. https://home.liebertpub.com/scd 18, 1501-1513 (2009).
22. Aizman, I., Tate, C. C., Mcgrogan, M. & Case, C. C. Extracellular Matrix Produced by Bone Marrow Stromal Cells and by Their Derivative, SB623 Cells, Supports Neural Cell Growth. doi:10.1002/jnr.22146.
23. Boura, J. S. et al. Direct head-to-head comparison of cationic liposome-mediated gene delivery to mesenchymal stem/stromal cells of different human sources: A comprehensive study. Hum Gene Ther Methods 24, 38-48 (2013).
24. Boura, J. S. et al. Evaluation of gene delivery strategies to efficiently overexpress functional HLAG on human bone marrow stromal cells. Mol Ther Methods Clin Dev 1, 14041 (2014).
25. Lesueur, L. L., Mir, L. M. & Andre, F. M. Overcoming the Specific Toxicity of Large Plasmids Electrotransfer in Primary Cells In Vitro. Mol Ther Nucleic Acids 5, e291 (2016).
26. Meir, Y. J. J. et al. A versatile, highly efficient, and potentially safer piggyBac transposon system for mammalian genome manipulations. The FASEB Journal 27, 4429-4443 (2013).
27. Ran, F. A. et al. Genome engineering using the CRISPR-Cas9 system. Nat Protoc 8, 2281-2308 (2013).
28. Meyerrose, T. E. et al. Lentiviral-Transduced Human Mesenchymal Stem Cells Persistently Express Therapeutic Levels of Enzyme in a Xenotransplantation Model of Human Disease. Stem Cells 26, 1713-1722 (2008).
29. Milone, M. C. & O'Doherty, U. Clinical use of lentiviral vectors. Leukemia 2018 32:7 32, 1529-1541 (2018).
30. Bae, J. et al. IL-2 delivery by engineered mesenchymal stem cells re-invigorates CD8+ T cells to overcome immunotherapy resistance in cancer. Nature Cell Biology 2022 24:12 24, 1754-1765 (2022).
31. Levy, O. et al. mRNA-engineered mesenchymal stem cells for targeted delivery of interleukin-10 bto sites of inflammation. Blood 122, e23-e32 (2013).

### Example 2

Non-Circulatory Cells Engineered with Circulatory Behavior for Infusion as a Cell Therapy

This invention is a new composition of matter through which mesenchymal/multipotent/medicinal stem/stromal/signaling cell/cells (MSCs) and their derivatives, induced pluripotent stem cells (iPSCs) and their derivatives, or other non-circulatory cells (e.g., HEK 293s) (hereafter called source cell) are engineered to behave like professional circulatory cells. This is accomplished through genetic engineering of the source cell to express a non-native surface profile of proteins that are characteristic of professional circulatory cells, resulting in an emergent homing behavior in the source cell.

Homing of a professional circulatory cell to a target tissue is accomplished by combinatorial specificity of surface proteins on the circulatory cell and the target cell, such as the cell adhesion cascade that occurs to recruit leukocytes to sites of inflammation^{1,2} (See FIG. 5). However, tissue resident cells such as MSCs and cell types generated in vitro such as iPSCs and HEK 293s lack key surface proteins used by professional circulatory cells to accomplish cell homing^{3,4}. This results in poor targeting when infused in vivo and reduced efficacy of the cell therapy⁵. This technology engineers a source cell to express a combination of these cell adhesion factors that synergize to recapitulate the cell adhesion cascade. This technology has been first developed in MSCs, leveraging recently developed methods to engineer MSCs with multiple genes then expand the engineered MSCs to clinically compatible doses (See Example 1). This could be extended to other cell types that lack circulatory behavior, such as iPSCs and HEK 293s. iPSCs are of particular interest because they could be engineered with the combination of factors and then differentiated into MSCs or another therapeutic cell type, resulting in an economically favorable source for cell therapy.

MSCs have been extensively studied as cell therapies and have been genetically engineered to express factors from the cell adhesion cascade³. However, these efforts have focused on expressing single genes in isolation (e.g., CXCR46) or a pair of genes in combination (e.g., PSLG-1 and sLex⁷) and have achieved marginal improvements without clinical significance^{5,8}. As the cell adhesion cascade achieves homing through multiple proteins, an approach only adding one or two factors failed to recapitulate the complex phenotype. A simplified example of our approach is shown in FIG. 6, wherein adding three genes (PSGL-1, FUT7, and CXCR4) in combination change the behavior of the cells from “hit and run” to “stay and play” in the context of targeting and ameliorating neuroinflammation.

In embodiments, a cell product can express the following combination of features. This list is not intended to be exhaustive, but instead would include but not be limited to:
i. Combinations of cell adhesion proteins such as PSGL-1, ESL-1, CD44, CD24, LFA-1, VLA-4, MAC-1, Talin, Kindlin, FUT7, FUT6, FUT4, ST3GAL6, ICAM-1, ICAM-2, LPAM-1, JAM-A, JAMB, CXCR1, CXCR2, CXCR4, CXCR7, CCR1, CCR2, CCR3, CCR4, CCR5, CCR7, EMR2, EMR3, CD97, MMP-1, MMP-2, and/or MMP-9
ii. Persistence modifications such as the CD47 “don't eat me signal” in combination with HLA knockout or knockdown⁹ and/or an oxidoreductase for hypoxic conditions¹⁰
iii. A small molecule inducible safety switch to be deployed in the case of an immune reaction or teratoma formation¹¹.
iv. A selectable marker to allow manufacturing (e.g., puromycin acetyl transferase or truncated CD34) and/or reporter for in-vivo tracking (e.g., iRFP713 or luciferase)

Together, these modifications should improve the circulatory behavior of the infused cell therapy and result in improved targeting to sites of inflammation. We found that adding both PSGL-1 and FUT7 were required to improve adhesion to recombinant P&E selectin, markers in inflammation (See FIG. 7).

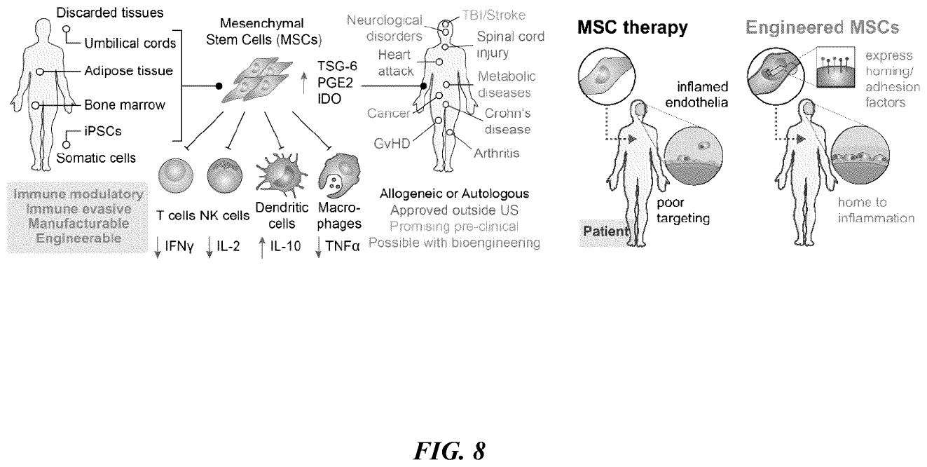

Mesenchymal stem/stromal cells (MSCs) have been extensively studied as allogeneic cell therapies across diverse indications⁵ due to their inherent immunomodulatory and regenerative properties¹², their immune evasive phenotype¹³, and their manufacturability from minimally invasive or discarded tissue sources¹⁴ (See FIG. 8). Despite this potential and promising pre-clinical studies, MSC-based therapies consistently fail in clinical trials across multiple indications¹⁵. Importantly, the overwhelming consensus from these trials is that MSC infusion (both autologous and allogeneic) is safe and well tolerated by patients, although there is little clinical benefit⁵. While these are significant challenges for traditional MSC cell therapy, we could view MSCs instead as an engineerable chassis. If it were possible to enhance this chassis with augmented potency, it could be safely deployed as an allogeneic therapy for a diverse array of indications. In particular, this technology could be deployed in traumatic indications such as wound healing or spinal cord injury, as well as inflammatory or autoimmune disorders, implant and transplant rejections, stroke, and heart attacks, many of which could benefit from improved homing to the site of injury.

Native MSCs have proven insufficient to treat the aforementioned indications, especially when infused intravenously. There has been some success when directly injected into the site of injury (i.e., Alosifel). Alternative strategies have deployed biomaterials to improve MSC persistence in vivo for immune modulation¹⁶. Finally, a recently developed strategy creates synthetic versions of cellular adhesion molecules¹⁷. However, in each case, these strategies fail to recapitulate the complex multifaceted biology of professional circulatory cells and will likely result in marginal clinical improvement.

In embodiments, we will validate the balance between incorporating enough of the proteins of cell adhesion to recapitulate the biological phenotype and the ultimate scalability of the cell product. Further, after successfully achieving cell homing, we can augment MSC potency with indication specific therapeutic factors using the same engineering strategy.

### REFERENCES CITED IN THIS EXAMPLE

1. Ley, K., Laudanna, C., Cybulsky, M. I. & Nourshargh, S. Getting to the site of inflammation: the leukocyte adhesion cascade updated. Nature Reviews Immunology 2007 7:9 7, 678-689 (2007).
2. Vestweber, D. How leukocytes cross the vascular endothelium. Nat Rev Immunol 15, 692-704 (2015).
3. Nitzsche, F. et al. Concise Review: MSC Adhesion Cascade—Insights into Homing and Transendothelial Migration. Stem Cells 35, 1446-1460 (2017).
4. Ullah, M., Liu, D. D. & Thakor, A. S. Mesenchymal Stromal Cell Homing: Mechanisms and Strategies for Improvement. iScience 15, 421-438 (2019).
5. Levy, O. et al. Shattering barriers toward clinically meaningful MSC therapies. Sci Adv 6, 1-19 (2020).
6. Cheng, Z. et al. Targeted migration of mesenchymal stem cells modified with CXCR4 gene to infarcted myocardium improves cardiac performance. Molecular Therapy 16, 571-579 (2008).
7. Levy, O. et al. mRNA-engineered mesenchymal stem cells for targeted delivery of interleukin-10 to sites of inflammation. Blood 122, e23-e32 (2013).
8. Damasceno, P. K. F. et al. Genetic Engineering as a Strategy to Improve the Therapeutic Efficacy of Mesenchymal Stem/Stromal Cells in Regenerative Medicine. Front Cell Dev Biol 8, 1-24 (2020).
9. Deuse, T. et al. Hypoimmunogenic derivatives of induced pluripotent stem cells evade immune rejection in fully immunocompetent allogeneic recipients. Nature Biotechnology 2019 37:3 37, 252-258 (2019).
10. Bae, J. et al. IL-2 delivery by engineered mesenchymal stem cells re-invigorates CD8+ T cells to overcome immunotherapy resistance in cancer. Nature Cell Biology 2022 24:12 24, 1754-1765 (2022).
11. Sahillioglu, A. C. & Schumacher, T. N. Safety switches for adoptive cell therapy. Curr Opin Immunol 74, 190-198 (2022).
12. Pittenger, M. F. et al. Mesenchymal stem cell perspective: cell biology to clinical progress. NPJ Regen Med 4, (2019).
13. Hare, J. M. et al. Comparison of allogeneic vs autologous bone marrow-derived mesenchymal stem cells delivered by transendocardial injection in patients with ischemic cardiomyopathy: The POSEIDON randomized trial. JAMA—Journal of the American Medical Association 308, 2369-2379 (2012).
14. Yin, J. Q., Zhu, J. & Ankrum, J. A. Manufacturing of primed mesenchymal stromal cells for therapy. Nat Biomed Eng 3, 90-104 (2019).
15. Galipeau, J. & Sensébé, L. Mesenchymal Stromal Cells: Clinical Challenges and Therapeutic Opportunities. Cell Stem Cell 22, 824-833 (2018).
16. Mao, A. S. et al. Programmable microencapsulation for enhanced mesenchymal stem cell persistence and immunomodulation. Proc Natl Acad Sci USA 116, 15392-15397 (2019).
17. Stevens, A. J. et al. Programming Multicellular Assembly with Synthetic Cell Adhesion Molecules. Nature 2022 1-3 (2022) doi:10.1038/s41586-022-05622-z.

### Example 3

Clinical-Scale Genetic Engineering of MSCs with Complex Genetic Cargos

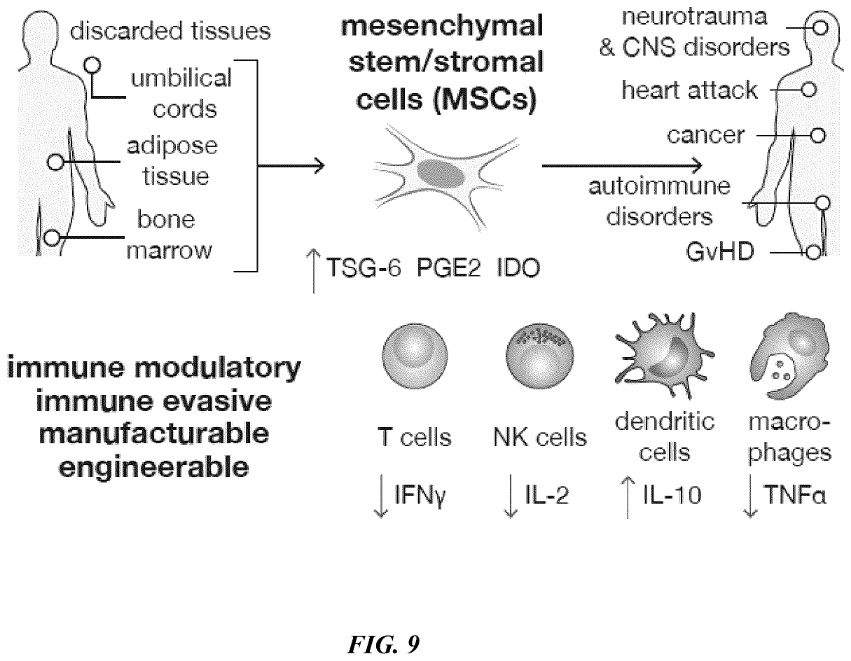

Referring to FIG. 9, a schematic showing applications of MSC cell therapies is provided.

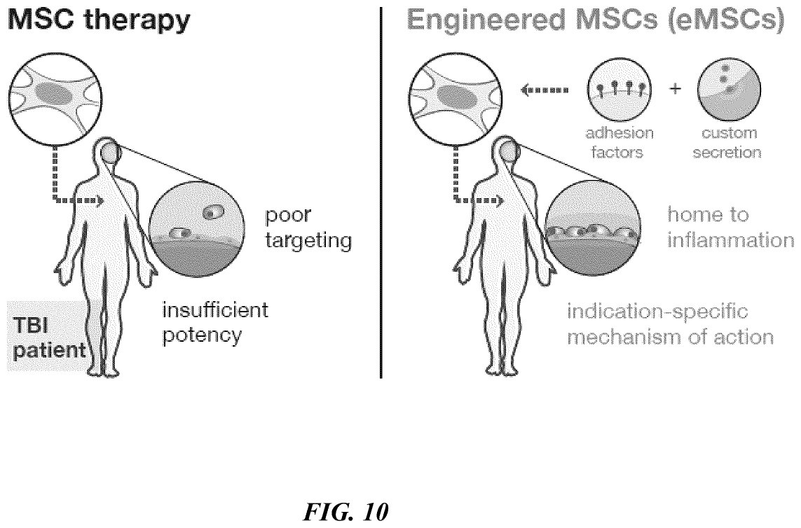

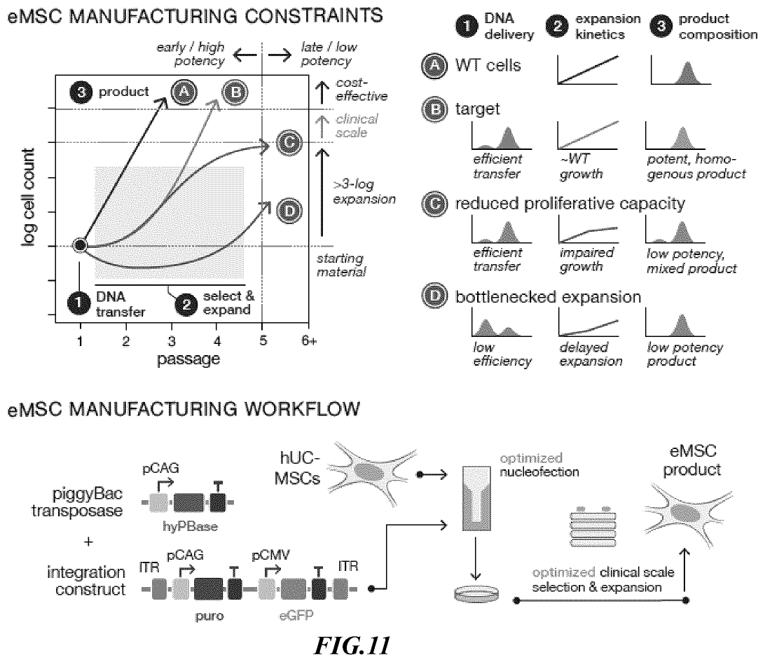

Referring to FIG. 10, a schematic showing challenges with MSC-based therapy is provided.

Referring to FIG. 11, data showing the framework for cell engineering is provided.

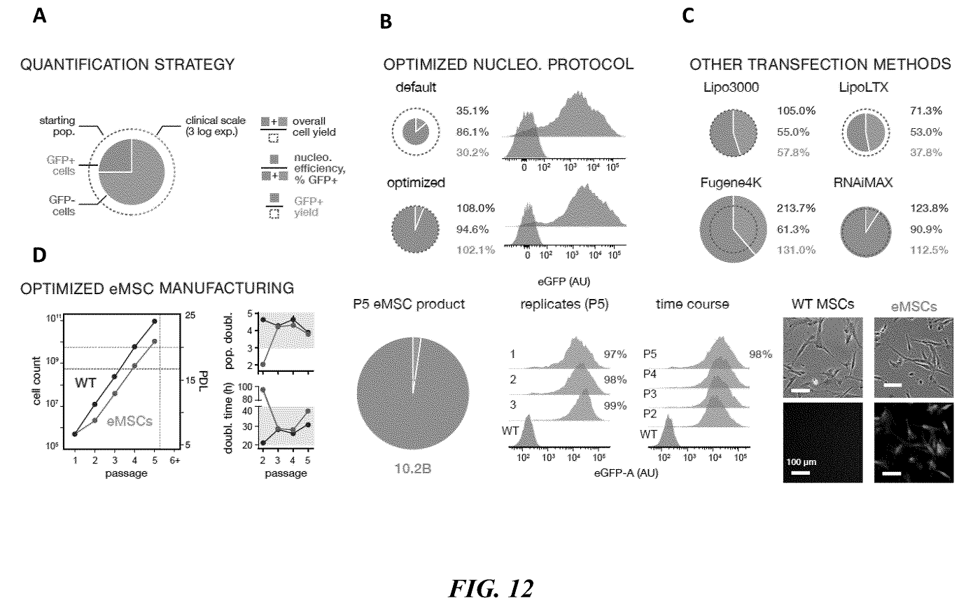

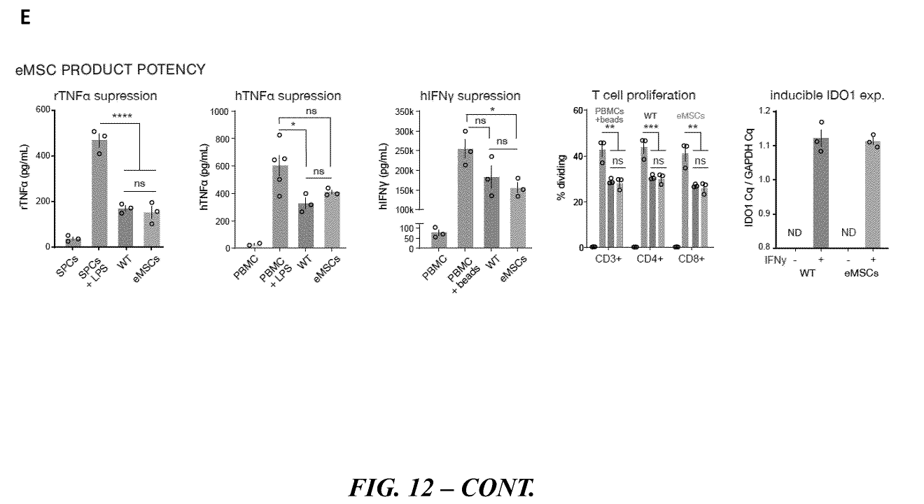

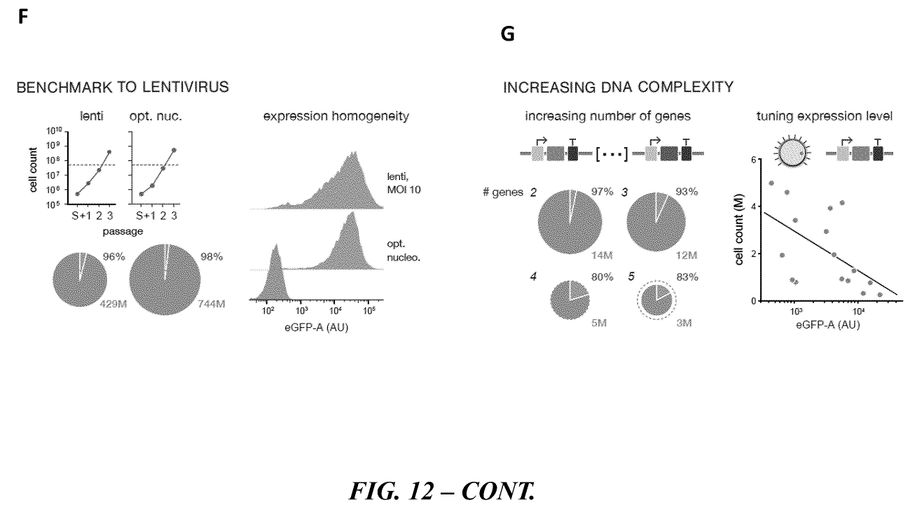

Referring to FIG. 12, data showing cGMP-compatible genetic engineering is provided. Panel A provides data showing the quantification strategy. Panel B provides data showing the optimized nucleofection protocol. Panel C provides data showing other transfection methods. Panel D provides data showing optimized eMSC manufacturing. Panel E provides data showing eMSC product potency. Panel F provides data showing benchmarks to lentivirus. Panel G provides data showing increasing DNA complexity.

Referring to FIG. 13, data showing multi-gene expression allowing for improved adhesion is provided.

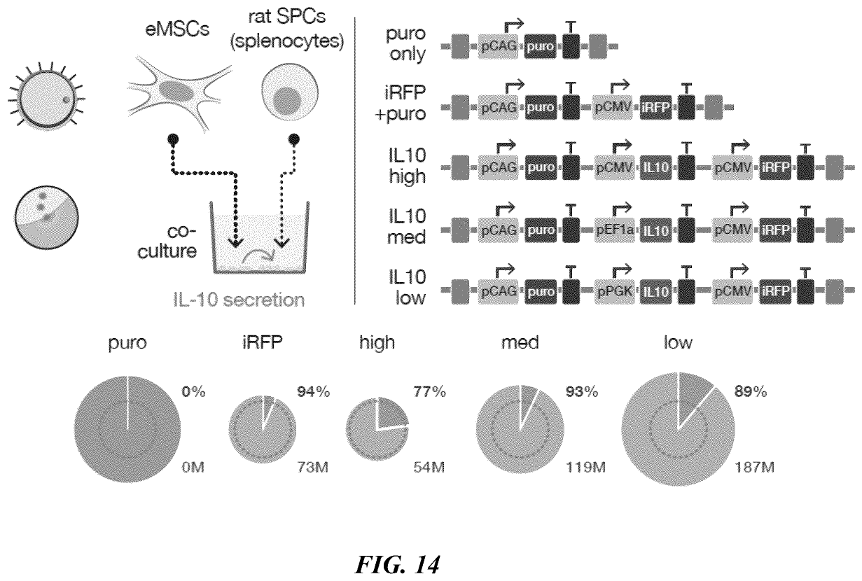

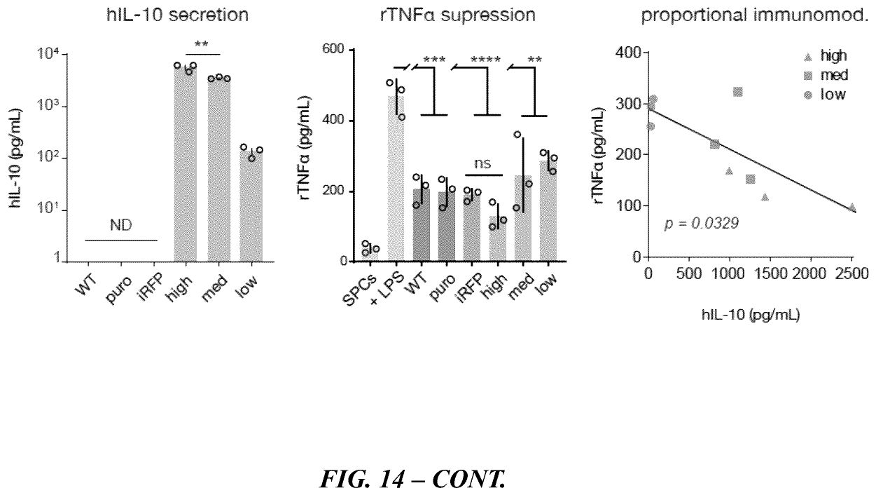

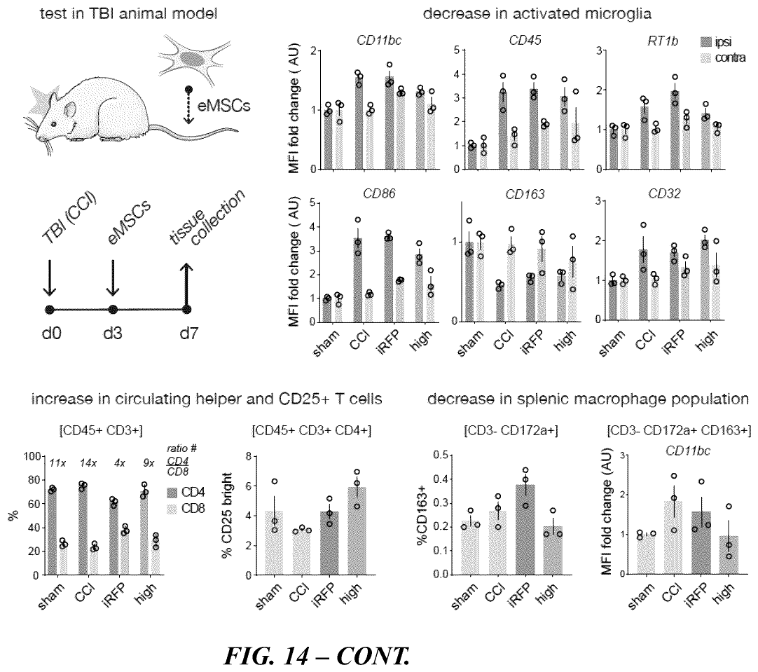

Referring to FIG. 14, data showing engineered secretion enhances potency is provided.

### Example 4

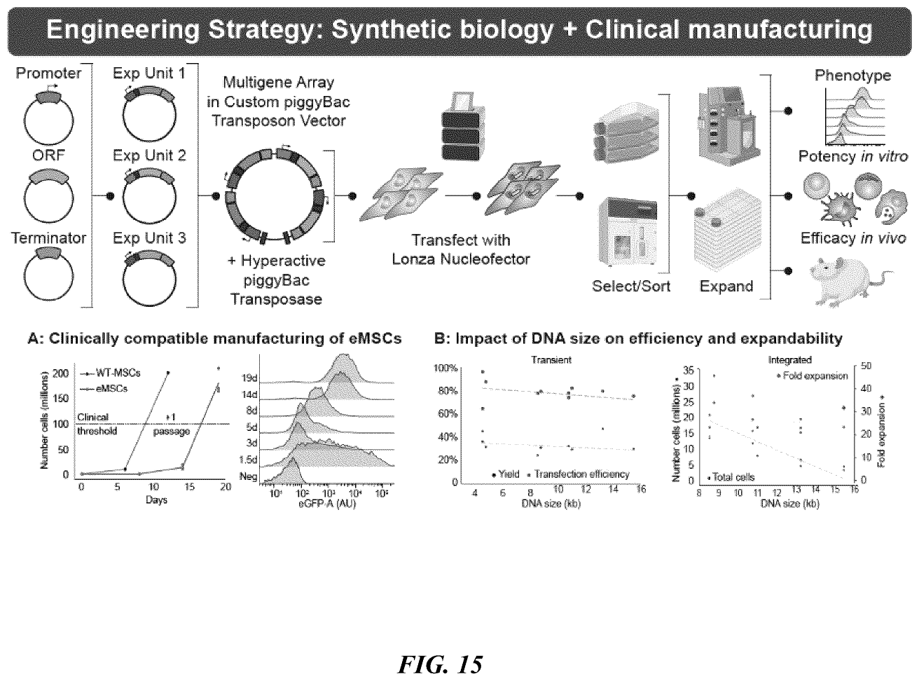

Referring to FIG. 15, data showing engineering strategy is provided. Multiparametric optimization results in eMSC numbers above the clinical threshold in only one extra passage (Panel A). The protocol is robust to increasing DNA size by adding expression units. Transient expression shows no significant impact in transfection efficiency or yield. DNA size reduces cell yield when integrated but expandability is unaffected (Panel B).

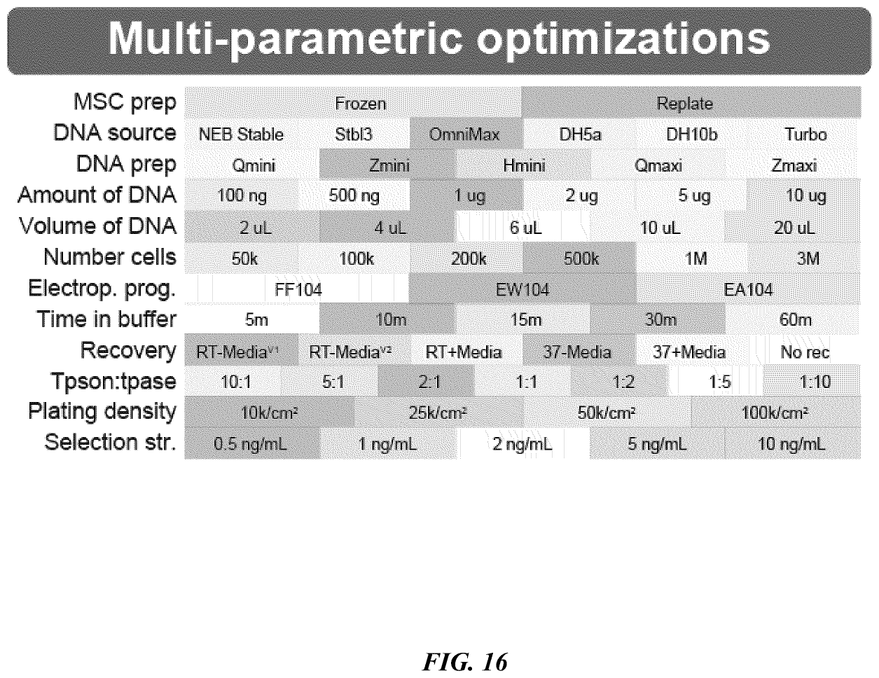

Referring to FIG. 16, data showing multi-parametric optimizations is provided. Multi-parametric optimizations spanning various axes of the manufacturing process improve the yield of eMSCs when combined.

### Example 5

Here, we show the full intended pathway. To our knowledge, no one has successfully installed this full pathway in MSCs and instead has focused on expression of “tethering” proteins, such as PSGI-1 and FUT7, or the “activation” proteins, such as CXCR4. Groups have overexpressed the missing protein ITGA4 in order to create the VLA-4 integrin. To our knowledge, simultaneous overexpression of ITGAL and ITGB2 to create the LFA-1 integrin has not been successfully done in MSCs.

Here, the key finding is that expression level is critical to successful manufacturing of MSCs overexpressing both PSGL-1 and FUT7. Further, the use of CMV promoters is toxic. Using CMV would be the first thing someone skilled in the art would use, and the precise expression level of the two factors that was necessary was not intuitive.

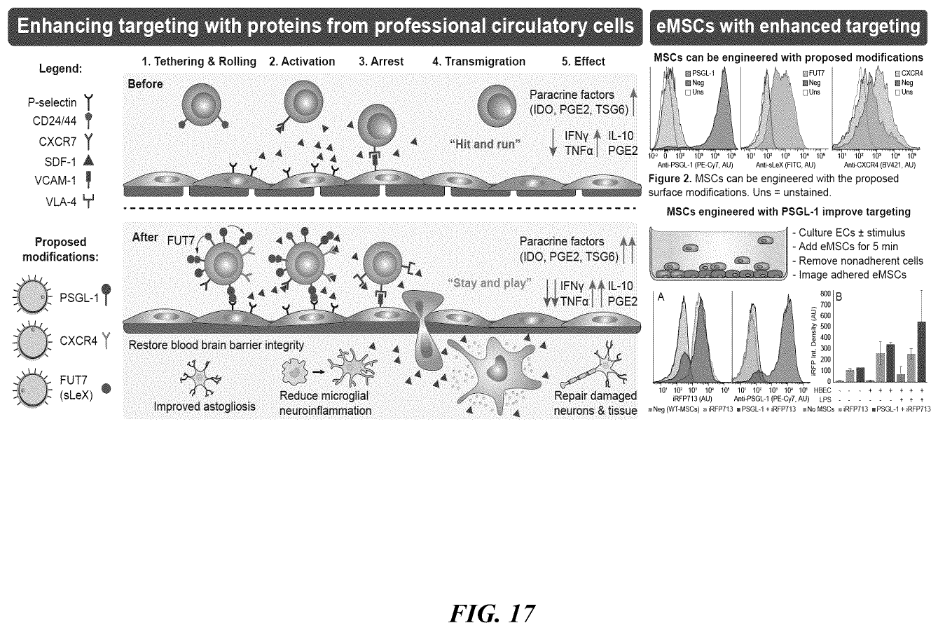

Referring to FIG. 17, data showing eMSCs with enhanced targeting is shown. MSCs can be engineered with the surface modifications described herein.

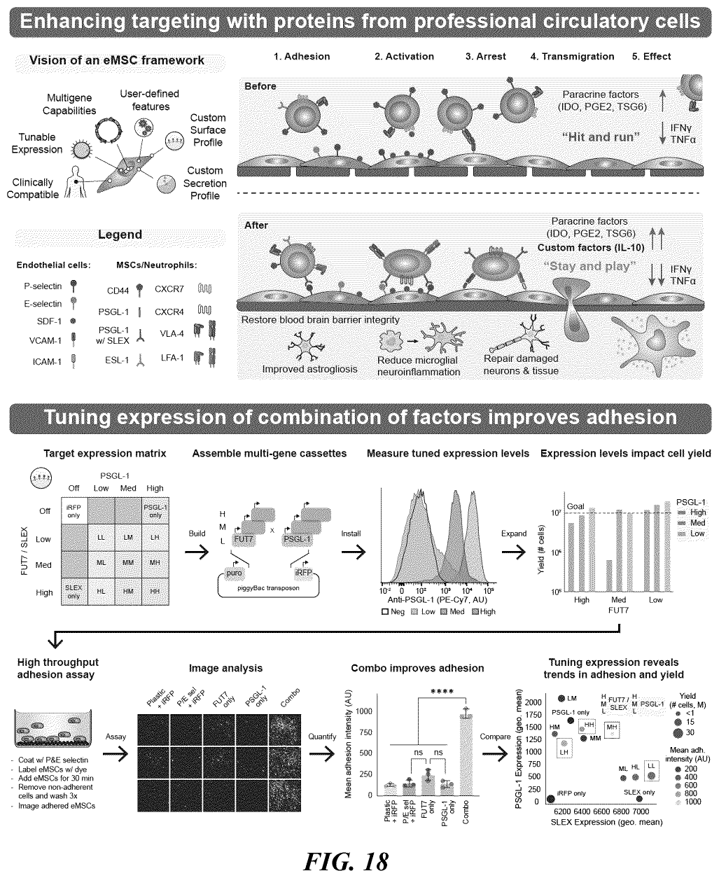

Referring to FIG. 18, data showing the workflow of design, assembly, characterization, quantification, and comparison of engineered adhesion is provided.

### Example 6

Clinically-Compatible Engineering of MSCs with Complex Genetic Cargoes

Mesenchymal stem/stromal cells (MSCs) have shown preclinical promise as a cellular therapy for a broad range of diseases and conditions, but have consistently failed in late-phase clinical trials. One possible strategy for improving the therapeutic potential of these cells is to create genetically engineered MSCs (eMSCs) harboring transgene expression programs that enhance functional potency. However, developing methods for genetically modifying MSCs that can support efficient and cost-effective manufacturing has been challenging, hindering eMSC translation. Here, we overcome this barrier by using multi-parameter optimization of DNA delivery, transgene selection, and product expansion techniques to develop a GMP-compatible manufacturing workflow capable of yielding a clinical-scale eMSC product. This workflow produces eMSCs with growth kinetics, phenotypic profile, and immunomodulatory properties similar to unmodified MSCs—an outcome that is reproducible across multiple donors and cell types. Importantly, we demonstrate that our cell engineering approach results in superior expression profiles compared to lentiviral and chemical transfection approaches. Furthermore, it allows for implementation of large, multi-transgene cargoes that can be reconfigured to generate products with fine-tuned non-native immunomodulatory function. This study establishes a framework for using complex genetic engineering approaches to develop next-generation eMSC therapies that can be readily scaled to clinical settings.

### INTRODUCTION

Cell-based therapies are an emerging pharmaceutical modality that harnesses the intrinsic functional properties of living cells to treat disease¹. Examples of FDA approved cell-based therapies include the use of patient-derived T cells to treat cancer², and the deployment of encapsulated islet cells to treat diabetes³. Despite these successes, many approaches that show therapeutic promise in preclinical studies demonstrate poor or inconsistent outcomes when they are scaled to clinical settings⁴. One potential strategy for enhancing both the efficacy and safety of cell-based therapy is to genetically modify cells by introducing ectopically expressed transgenes or synthetic regulatory circuits⁵. Cells could be engineered with enhanced secretion of therapeutic factors, or programmed with disease marker-specific sense-and-respond function. However, genetic modification can escalate the cost of manufacturing cell-based therapies by lowering yield, or by requiring more stringent quality control and regulatory compliance measures⁶⁻⁹.

Mesenchymal/multipotent stem/stromal cells (MSCs) offer a notable example of the potential for using genetic engineering to enhance therapeutic capacity. MSCs exhibit intrinsic immunomodulatory and regenerative properties¹⁰, immune evasion^{11,12}, and are readily isolated from minimally invasive or discarded tissue sources¹². As a consequence, they have been studied as a prospective treatment for diverse indications, including autoimmune disease, inflammatory disorders, orthopedic injury, and neurological trauma^{4,13}. However, despite many positive pre-clinical results and a strong record of clinical safety, MSC-based therapies have consistently failed in large-scale clinical trials due to inconsistent potency or poorly defined mode of action^{14,15}. Genetically engineered MSCs (eMSCs) have the potential to address these failures, for instance, by introducing transgenes that augment functional potency by enhancing immunomodulatory function¹⁶ or redirecting cell tropism^{17,18}. However, despite numerous attempts^{4,16,19-23}, no eMSC therapeutic products have been translated into FDA approved therapies.

A key reason for this limitation is the difficulty in manufacturing an eMSC product²⁰. DNA transfer and selection of transgenic cells can create manufacturing bottlenecks that extend the number of population doublings required to reach a clinical-scale product, leading to loss of favorable functional properties associated with early passage MSCs, (e.g., their secretory profile)²⁴⁻²⁶ and increase tumorigenic risk²⁷. A second contributing factor is that most eMSC therapies involve overexpression of only a single transgene. To address the multi-faceted nature of the disorders and injuries that eMSCs are deployed to treat, or to implement synthetic regulatory circuits that dynamically respond to disease state⁵, it may be beneficial to engineer larger genetic programs that contain multiple transgenes^{1,13}. Thus, there is a need to develop low-cost, clinically scalable eMSC manufacturing processes that can efficiently generate products harboring larger and more complex genetic cargo.

Here, we address this deficit by developing a versatile cell engineering workflow that allows manufacturing of clinically-compatible eMSC products engineered with multi-gene cargos. Our approach uses nucleofection to deliver transposon-based DNA vectors to human umbilical cord MSCs (hUC-MSCs). Following extensive optimization of conditions for DNA delivery, eMSC selection, and product expansion, we show that our workflow can generate a GMP-compatible, clinical-scale cell product that exhibits homogenous transgene expression and phenotypic properties similar to unmodified, low passage MSCs. The workflow can be robustly generalized across multiple donors and tissue sources, and exhibits key advantages over other commonly used eMSC engineering approaches. We demonstrate production of eMSC products harboring large and complex DNA constructs (>12 kb), including programs encoding IL-10 production that can be tuned to differentially modulate inflammatory response.

### Results

Developing an eMSC Manufacturing Strategy

A manufacturing strategy that supports clinical translation of eMSCs should yield a cell product with the following features: 1) the DNA cargo harbored by the cells should be large enough to encode multiple transgenes; 2) transgene expression should be homogenous to ensure functional similarity across the cell population; 3) expression should show long-term stability; 4) genetic manipulation and expansion should not impact the product safety profile; 5) the overall cost of generating the product from starting materials should be minimized (FIG. 19A, left). Generating a product with these features requires navigating multiple tradeoffs. For example, while DNA delivery approaches that involve genomic integration (e.g., lentiviral transduction or transposon-mediated integration) facilitate stable expression, they also increase safety risks due to oncogenic transformation^{28,29}. Similarly, using antibiotic selection to achieve homogenous transgene expression may bottleneck the expansion process, resulting in longer outgrowth and higher manufacturing costs.

Consideration of the various strategies that have been used to manufacture eMSC products for the clinic underscore the challenges these tradeoffs present. Two general DNA delivery approaches have been employed (FIG. 19A, right): 1) non-viral, which include lipofection of DNA^{30,31} or RNA^{32,33}, as well as nucleofection of DNA; and 2) viral, which include adenovirus³⁴, γ-retrovirus^{35,36}, and lentivirus³⁷. Lipofection and nucleofection can be used to transfer large capacity DNA vectors but yield heterogeneous products³⁸. On the other hand, RNA delivery is efficient, generates a more homogenous product, and carries low safety risk³⁹, but its cargo size is typically limited to a single gene per mRNA⁴⁰. For all transient approaches, the requirement that nucleic acids be delivered at large scale following product expansion can drive up costs³⁹. Viral approaches allow stable transgene expression when transduced early in the manufacturing process, but suffer from limited cargo capacity and variable risk profiles, and can also introduce costs due to the requirement to manufacture and qualify viral particles⁶⁻⁸.

In developing our eMSC manufacturing approach, we sought to navigate these tradeoffs while working within a set of intrinsic technical and logistical constraints (FIG. 19B, left). First, to ensure phenotypic homogeneity and maintain expansion potential^{6,41,42}, early passage MSCs should be used as a starting point for DNA delivery. Second, while MSCs can be expanded for ˜50 population doublings before undergoing senescence, current best practices use 20-25 (˜5 passages) to prevent loss of phenotype and avoid genetic instability and tumorgenicity^{20,27}. Finally, to achieve clinical scale, 1M cells must be expanded by >3 logs within the 5-passage window to obtain at least 1B cells, enough for a single dose for a 100 kg patient (10⁷ cells/kg for a 100 kg) 6,43. Exceeding this scale with another log expansion can achieve a cost-effective status by producing ten times the number of doses per manufacturing run, lowering the cost per dose. Working within these constraints requires efficient DNA delivery and expansion techniques (FIG. 19B, right). Inefficient delivery, ineffective selection of transgenic cells, or transgene toxicity can lead to delayed or impaired growth kinetics. This can necessitate additional passages to reach clinical scale, potentially resulting in a product with diminishing potency and a compromised safety profile.

The manufacturing approach we developed was designed to avoiding the failure modes described herein while supporting production of an eMSC product harboring complex multi-gene cargos (FIG. 19C). To facilitate rapid cargo construction, we leveraged a modular, hierarchical DNA assembly scheme⁴⁴ in which individual expression units are constructed from genetic part inputs and then assembled into a piggyBac vector for efficient transfer and transposon-mediated genomic integration^{7,45}. A puromycin expression unit is included in the assembly to facilitate selection of positive integrants (FIG. 27). We selected nucleofection as our DNA delivery method due to its reported ability to deliver large DNA constructs in an efficient, non-toxic, and GMP-compliant manner³⁸. As a cell source, we chose early passage human umbilical cord MSCs (hUC-MSCs), which are derived from a highly-accessibly discarded tissue source and have excellent expansion potential⁴⁶. We observed best practices of clinical manufacturing during cell isolation and handling, which included the use of a GMP-compatible, xeno-free media formulation (αMEM supplemented with human platelet lysate)^{47,48}. We then tested whether we could identify optimized nucleofection, selection, and expansion conditions that could support manufacturing of an eMSC product that maximizes the features described in FIG. 19A.

Optimizing eMSC Nucleofection

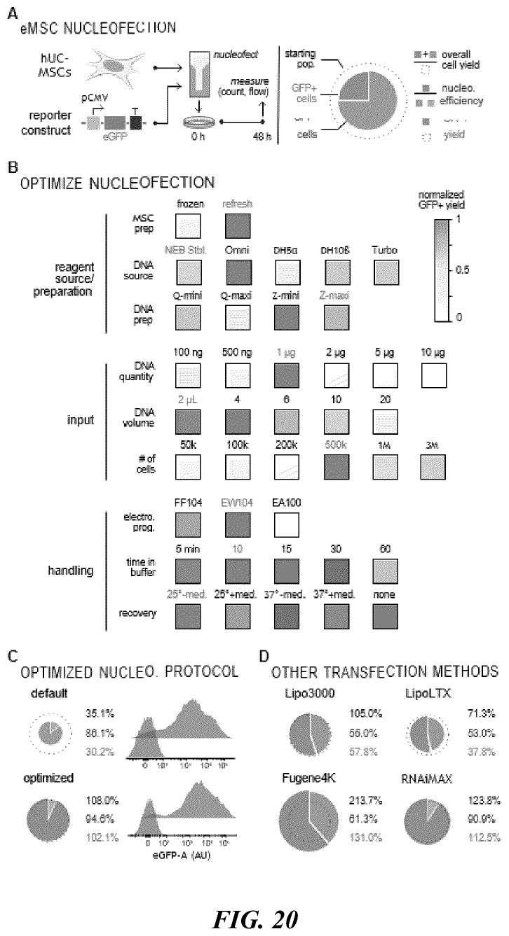

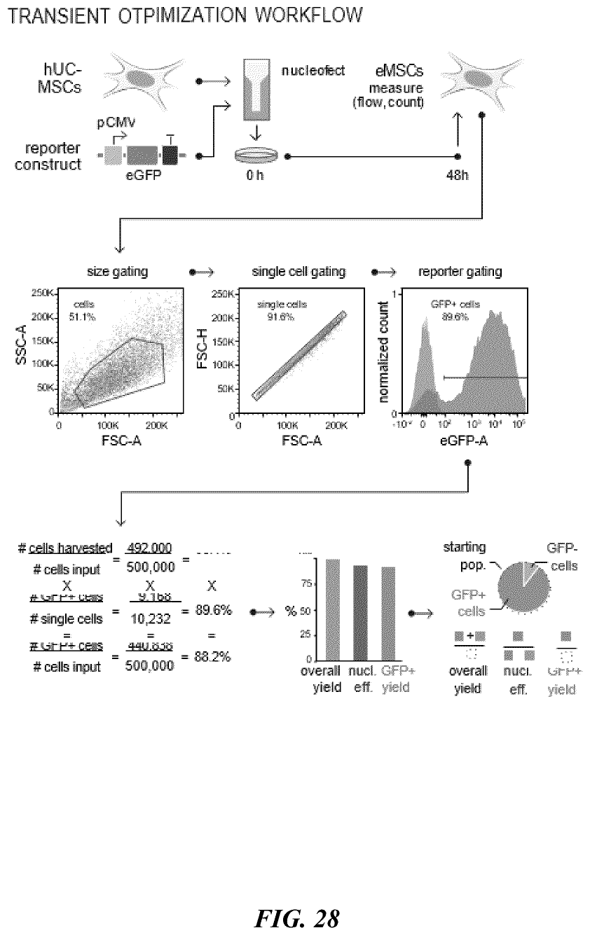

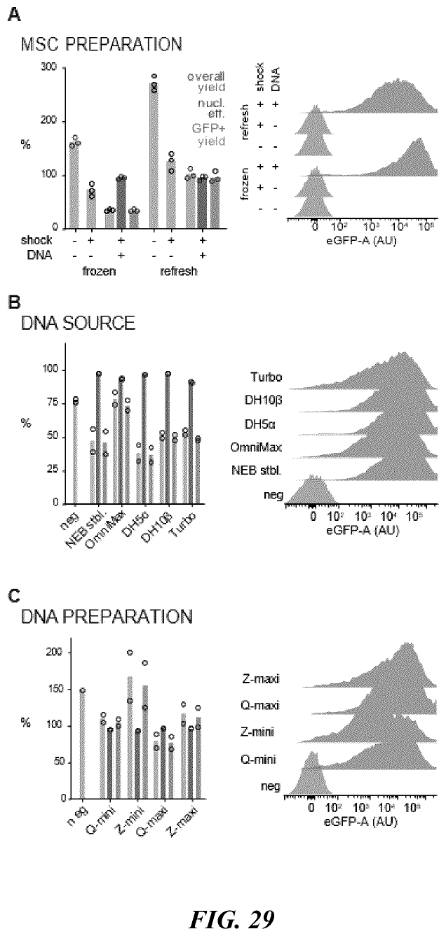

To quickly explore nucleofection conditions, we developed an assay in which hUC-MSCs were transiently transfected with an eGFP reporter plasmid, plated on plastic for 48 h to allow recovery, counted, and then assayed for eGFP fluorescence by flow cytometry (FIG. 20A, FIG. 28). This approach allows quantification and optimization of the yield of cells expressing GFP as a proportion of the starting population (GFP+), and also allowed us to assess nucleofection toxicity (by measuring overall cell yield) and reporter transfection efficiency (ratio of GFP+ to GFP-cells) (FIG. 20A, right). As a first step, we tested variables associated with cell source and DNA preparation (FIG. 20B, top). We found that hUC-MSCs refreshed in culture for 24 h improved GFP+ yield over cells that were directly thawed (FIG. 29A), likely due to recovery from freeze damage. Since different DNA preparation methods can result in variable transfection efficiency due to factors such as purity and endotoxin contamination⁴⁹ we evaluated the effects of various commercially available DNA preparation kits and E. coli cloning strains on GFP+ yield. While we found that Zymo Miniprep kits and OmniMax (Invitrogen) cells resulted in the best yields (FIG. 29B,C), for the remainder of the study we used NEB Stable cells, which performed comparably but are able to maintain large, complex plasmids without recombination⁵⁰, and Zymo Maxiprep kits due to their ability produce large quantities of DNA.

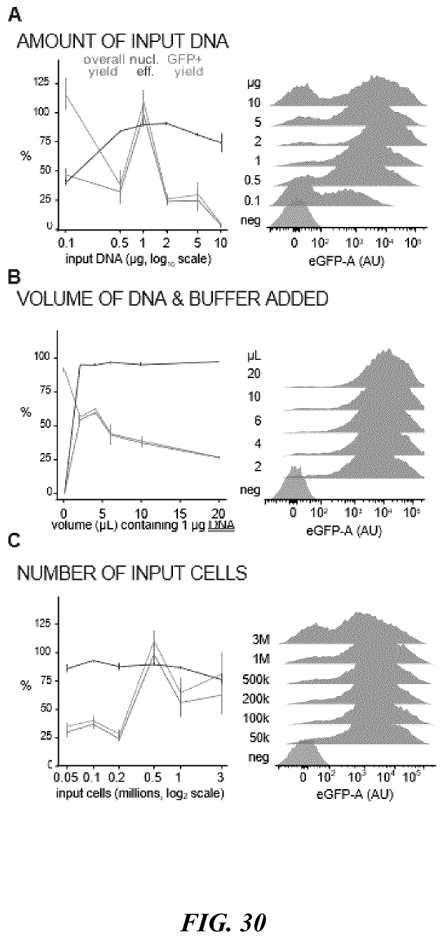

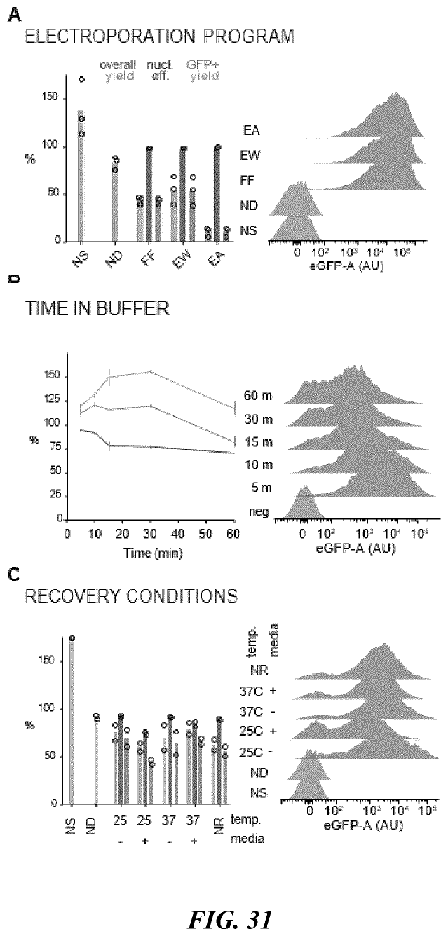

Next, we optimized the relative ratios of inputs to the nucleofection reaction-DNA quantity, carrier volume, and cell number—across normalized cell source and DNA preparation conditions (FIG. 20B, middle). We found that 1 μg of DNA produced an optimum GFP+ yield; lower amounts produced a heterogeneous product while higher amounts showed better nucleofection efficiency but also toxicity (FIG. 30A). The volume of added DNA impacted GFP+ yield but not transfection efficiency, with 2-4 μL maximizing yield (FIG. 30B). We also observed a local optimum of 500k cells that facilitated the highest GFP+ yield (FIG. 30C). Finally, we compared two Amaxa nucleofection programs, EW104 and FF104, that have different electrical parameters. While both programs resulted in similar GFP+ yields, EW104 demonstrated lower toxicity (FIG. 21B, bottom and FIG. 31A). We also tested the effect of recovery conditions, including time spent in buffer and recovery temperature, but found they had little impact on efficiency or yield (FIG. 31B,C).

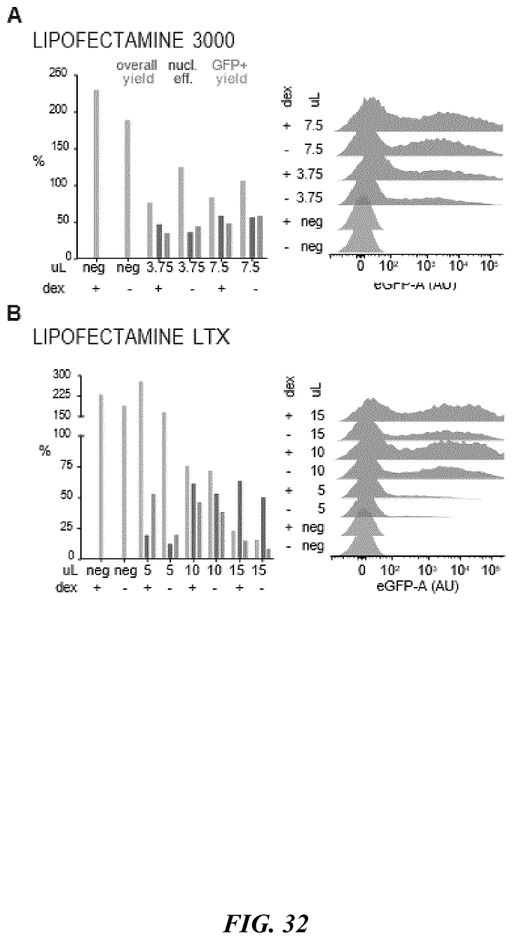

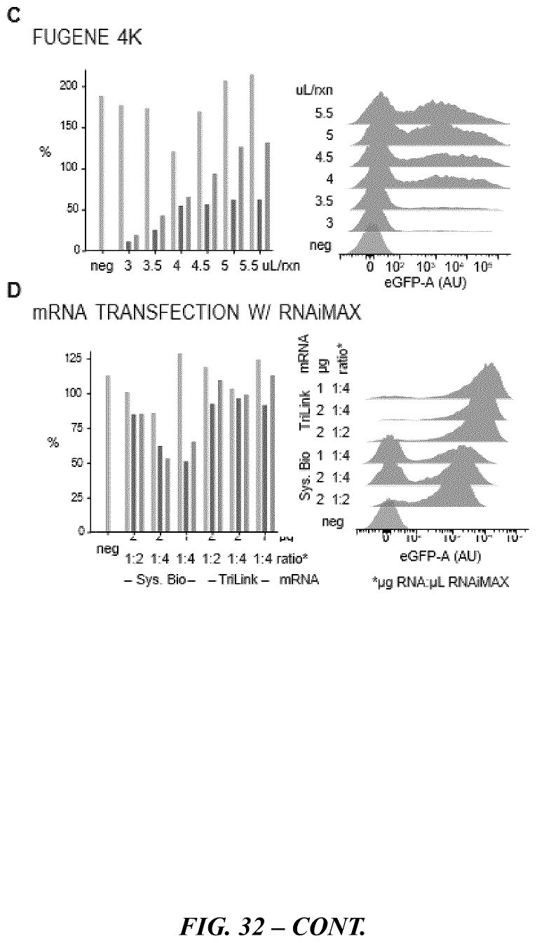

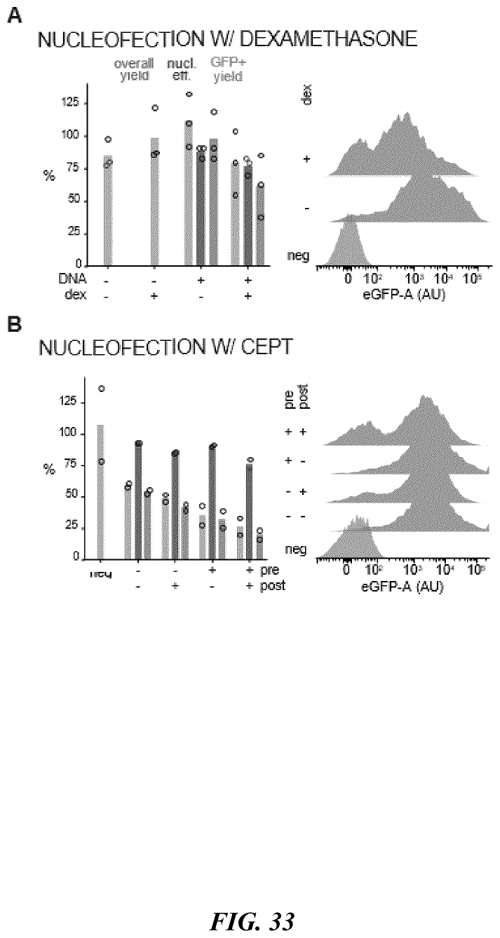

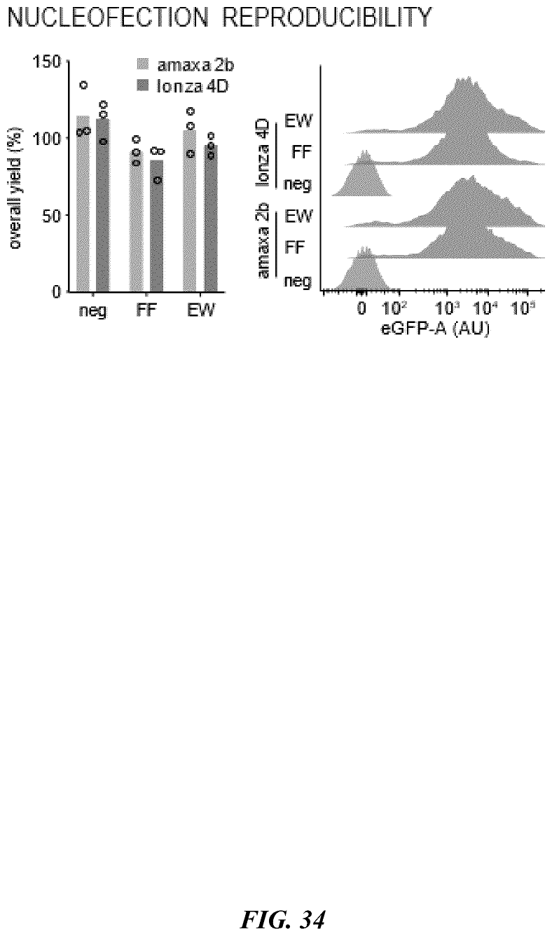

We consolidated the best individual nucleofection conditions into single protocol and tested it against a set of default manufacturer-recommended conditions (FIG. 20C). This optimized protocol resulted in a 102% GFP+ yield compared to the 30.2% for the default protocol. Furthermore, the consolidated protocol outperformed commonly used transient transection reagents (Lipofectamine 3000, Lipofectamine LTX, and Fugene 4K) (FIG. 20D), demonstrating either higher efficiency (compared to Fugene 4K and Lipofectamine 3000) or lower toxicity (Lipofectamine LTX) (FIG. 32A-C). Additionally, our protocol showed a similar level of efficiency, homogeneity, and yield compared to RNAiMAX, an mRNA delivery reagent that has been used to deliver single-gene payloads (FIG. 32D) 32. We also assessed the impact of dexamethasone⁵¹ and CEPT⁵² on transfection outcomes. While we confirmed that dexamethasone improves Lipofectamine-mediated transfection (FIG. 32A-C), we found that neither dexamethasone nor CEPT improved nucleofection efficiency or yield (FIG. 33). Finally, to assess the reproducibility of our protocol, we confirmed the optimized nucleofection conditions were consistent across two different nucleofectors (Amaxa 2b and Lonza 4D) for both electroporation programs used in this study (FF104 and EW104) (FIG. 34).

Optimizing eMSC Selection and Expansion

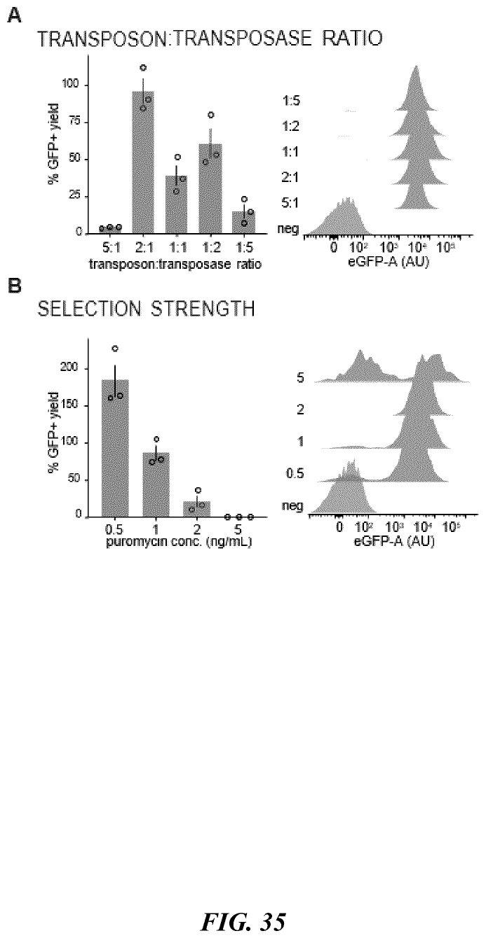

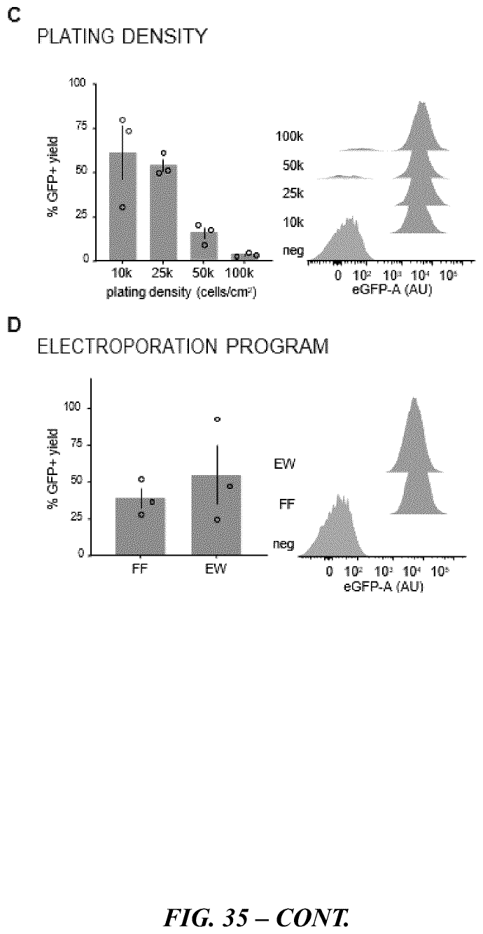

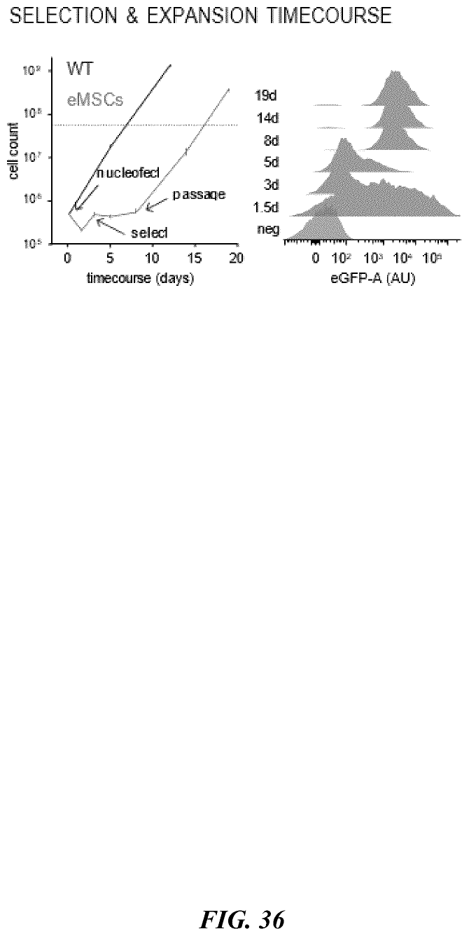

With an efficient nucleofection protocol in-hand, we next sought to optimize conditions of eMSC selection and expansion. We modified our assay to include nucleofection of a piggyBac reporter construct containing EUs encoding eGFP and puromycin resistance (FIG. 21A). We plated nucleofected cells on plastic for 72 h, added puromycin to select for integrants, and expanded cells for 3 passages to assess whether they could achieve growth rates needed to support expansion to a clinical scale, which for this assay corresponds to a 100-fold increase in ˜3 passages. Cells subjected to various selection conditions, including transposon: transposase plasmid ratio, puromycin concentration, and plating density (FIG. 21B). We found that a 2:1 molar ratio of transposon:transposase (FIG. 35A) and a puromycin concentration of 0.5 ng/mL resulted in the best GFP+ yield (FIG. 35B). We also observed that lower plating densities improved yield (FIG. 35C) with 10k cells/cm² producing a higher GFP+ yield but greater variability than 25k cells/cm². Additionally, we verified that program EW104 maintained superior GFP+ yield post-selection compared with FF104 (FIG. 35D). We assessed culture dynamics under these optimized selection conditions, observing an initial loss of overall yield at 48 h and broadly distributed GFP expression. This was followed by recovery to a unimodal GFP expression distribution by 8 d that was maintained after subsequent passages (FIG. 36).

We next tested several expansion regimes (measured from the first to the third passage) to determine which approach could facilitate the target 100-fold increase. These included expansion under constant puromycin selection, selection for one passage only (“pulsed”), flow sorting at 72 h instead of performing selection, and expanding without selection or sorting (FIG. 21C). While cells in each regime achieved the target growth rate and showed similar GFP+ yields, we observed that constant selection showed the most homogenous GFP expression profile, and the pulsed regime exceeded the target yield.

Demonstrating Clinical-Scale eMSC Manufacturing

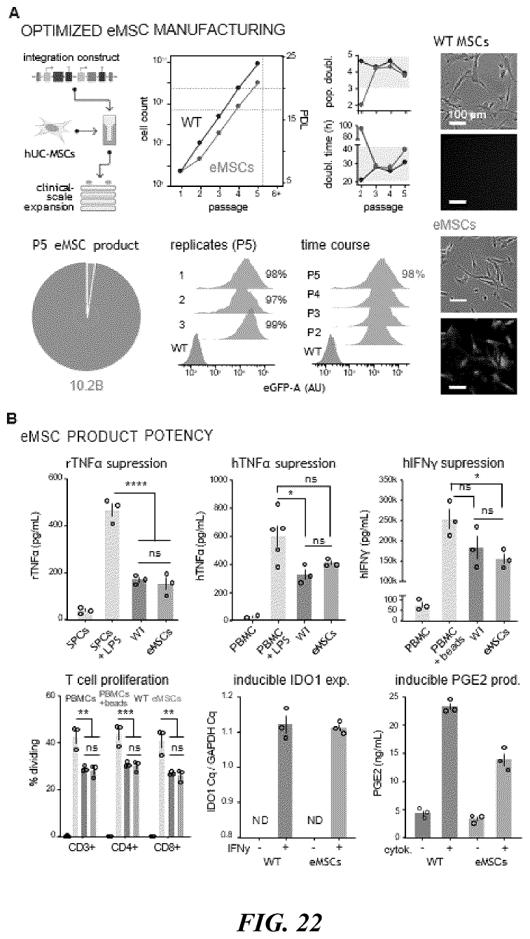

To assess whether we could combine our optimized integration, selection, and expansion methods into a single manufacturing workflow capable generating a clinically compatible eMSC product, we nucleofected our piggyBac reporter construct into 500K single-passage hUC-MSCs. This number corresponds to approximately 0.25% of the 200M hUC-MSCs obtained from a typical umbilical cord^{53,54}, which contains a quantity of starting material that, when expanded by at least 3 logs, could support the generation of >200 doses per cord (1B cells/dose). We expanded the cells in a multi-layer 2D culture format commonly used for GMP manufacturing of MSC therapy products (FIG. 22A). This workflow yielded ˜10.2 billion cells (˜4.31 log expansion) in just 4 passages, exceeding the target 3-log threshold by >10×. When scaled to a full cord, this represents a cost-effective approach for generating >4000 doses, sufficient for large, multicenter Phase III clinical trials. Even at this scale, post-selection growth kinetics were similar to WT MSCs expanded in parallel, and the final transgene expression profile was unimodal (˜1.5 log expression range), reproducible, and stable without continuous selection. Compared with expanded WT cells, cells in the manufactured eMSC product showed similar morphology (FIG. 22A, right). Furthermore, both WT and eMSC cell products have an immunophenotype that meet ISCT standards and show low expression of HLA-DR, indicating low immunogenicity (Table 3). We evaluated the eMSC product with a battery of immune modulation assays (FIG. 22B), demonstrating that compared to expanded WT cells, eMSCs show similar paracrine suppression of rat TNFα in a splenocyte co-culture, as well as suppression of human TNFα, IFNγ, and T-cell proliferation when co-cultured with human PBMCs. eMSCs also demonstrated IDO1 induction similar to WT cells, though PGE2 induction was diminished. Taken together, our results demonstrate that systematic optimization of cell engineering and culture techniques can generate a cost-effective manufacturing protocol capable of producing a clinical-grade eMSC product with a homogenous transgene expression profile without compromising innate potency.

TABLE 3
Phenotype of GMP-compatible engineered MSCs

| % | GFP+ | CD34+ | CD45+ | CD73+ | CD90+ | HLA-DR+ |
|---|---|---|---|---|---|---|
| WT P1 | 0.01 | 0.60 | 0.16 | 99.80 | 100.00 | 0.65 |
| WT P5 A | 0.01 | 0.00 | 0.00 | 100.00 | 100.00 | 0.01 |
| WT P5 B | 0.01 | 0.01 | 0.01 | 100.00 | 100.00 | 0.02 |
| WT P5 C | 0.01 | 0.03 | 0.00 | 100.00 | 100.00 | 0.02 |
| eMSC P5 A | 98.20 | 0.06 | 0.01 | 100.00 | 100.00 | 0.01 |
| eMSC P5 B | 96.80 | 0.01 | 0.00 | 100.00 | 100.00 | 0.07 |
| eMSC P5 C | 95.30 | 0.12 | 0.00 | 100.00 | 100.00 | 0.01 |

### Impact of Cell Source on Manufacturability

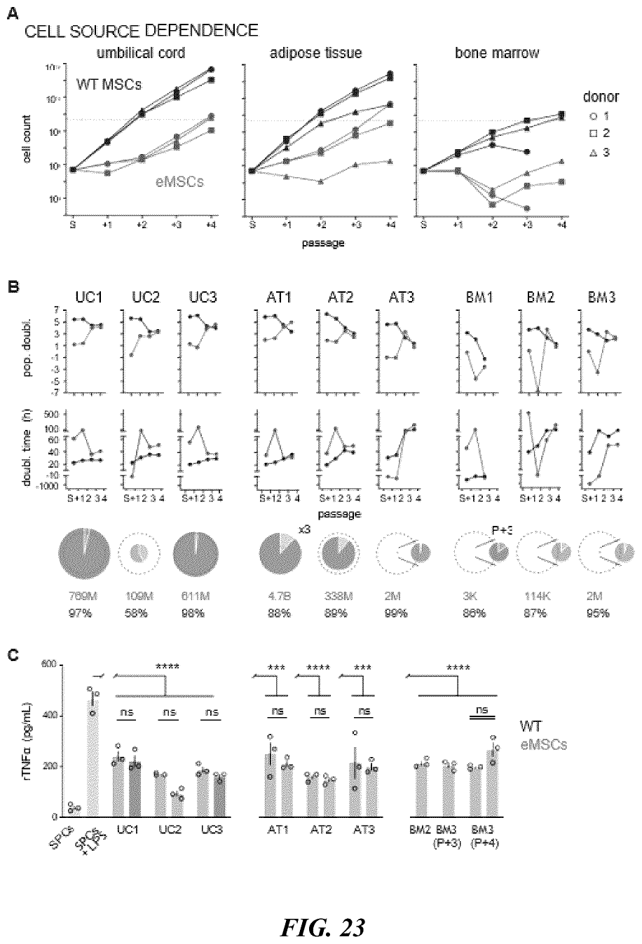

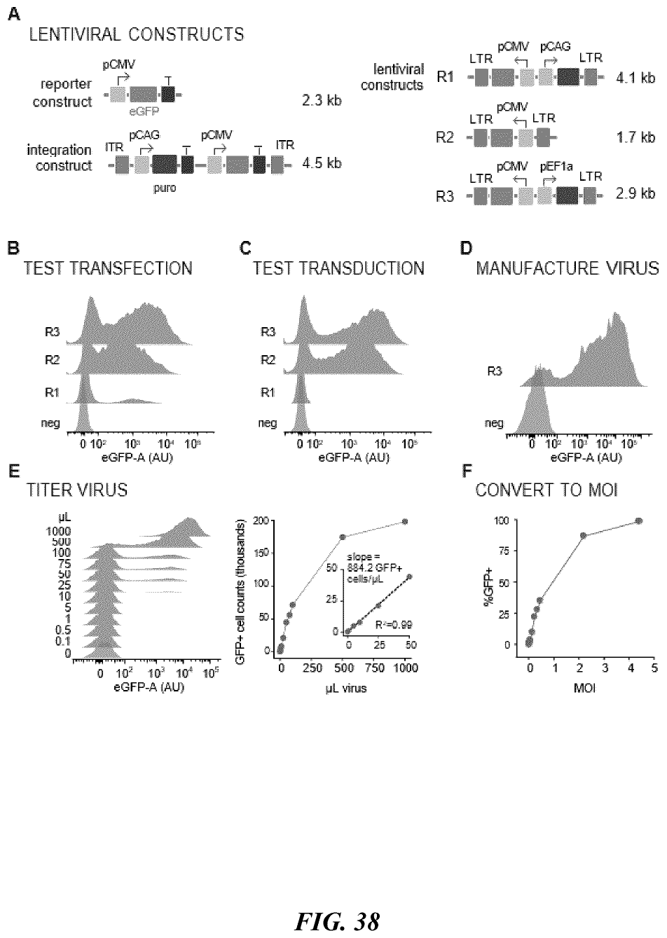

We next assessed the robustness and generalizability of our manufacturing workflow by comparing our hUC-MSC-derived eMSCs to those manufactured from adipose tissue (hAT-MSCs) and bone marrow (hBM-MCSs), testing three replicate donors from each source (FIG. 23A). All harvested cell samples were confirmed as MSCs per ISCT criteria (Table 4). We evaluated expansion kinetics, GFP+ yield (FIG. 23B and FIG. 38), and immune modulation (FIG. 23C) for all samples after 3-4 passages. We observed that expansion kinetics for each eMSC sample mirrored that of unmodified cells from its corresponding donor; cells with impaired growth from one hAT-MSCs (AT3) and hBM-MSCs [BM1] donors produced eMSCs that failed to expand. hUC-MSC-derived eMSCs demonstrated near-WT growth kinetics, as did 2 out of 3 hAT-MSC-derived samples. Unmodified hBM-MSCs did not expand as rapidly a hUC- or hAT-MSCs, only narrowly exceeding the clinical threshold⁴⁶. Interestingly, these discrepancies were not reflected in the immune modulation data, as sample with impaired performed similarly regardless of expansion efficiently.

TABLE 4
Phenotype of MSCs from multiple donors and cell types

| | | | | | HLA- |
|---|---|---|---|---|---|
| % | CD34+ | CD45+ | CD73+ | CD90+ | DR+ |
| U1 7204 | 0.60 | 0.16 | 99.8 | 100.0 | 0.65 |
| U2 4425 | 0.53 | 0.26 | 99.6 | 100.0 | 1.82 |
| U3 9260 | 0.94 | 0.47 | 99.4 | 100.0 | 0.47 |
| A1 1314 | 0.53 | 0.47 | 98.0 | 100.0 | 1.49 |
| A2 1271 | 0.23 | 0.32 | 94.7 | 100.0 | 1.48 |
| A3 5713 | 0.55 | 0.68 | 92.9 | 100.0 | 1.66 |
| B1 3305 | 1.17 | 2.09 | 94.6 | 100.0 | 2.60 |
| B2 5231 | 1.38 | 1.92 | 95.4 | 99.9 | 3.20 |
| B3 5190 | 2.25 | 2.42 | 93.4 | 100.0 | 7.33 |

### Comparison to Lentiviral Transduction

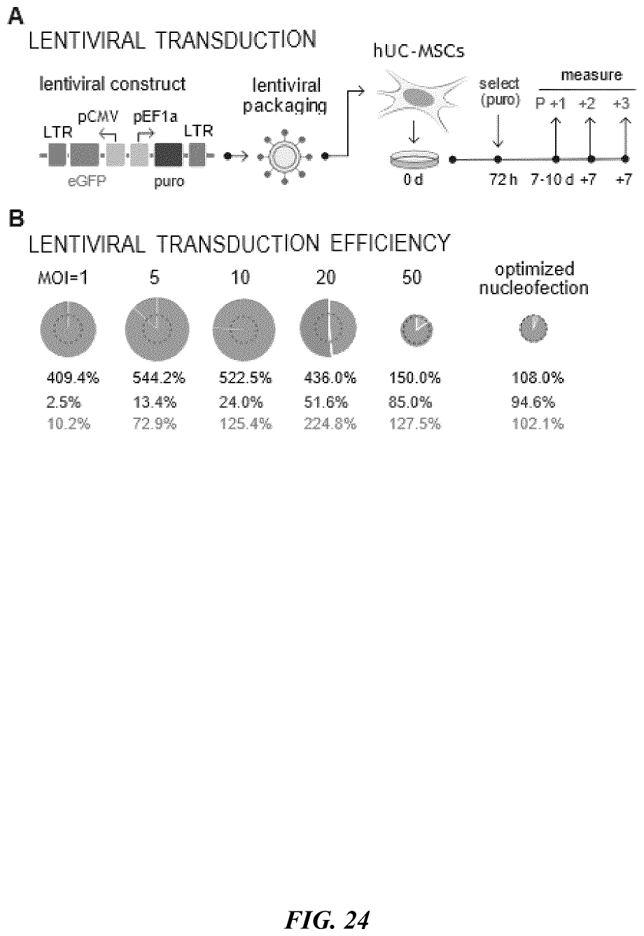

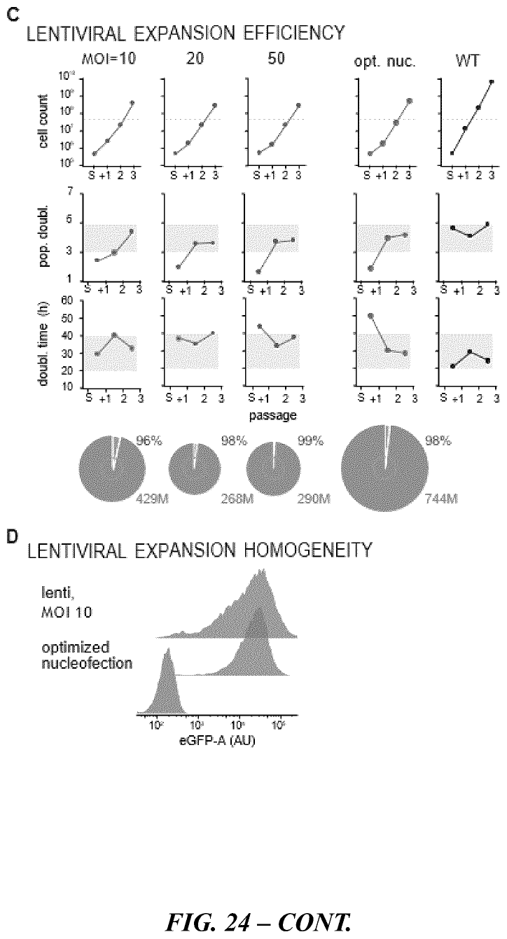

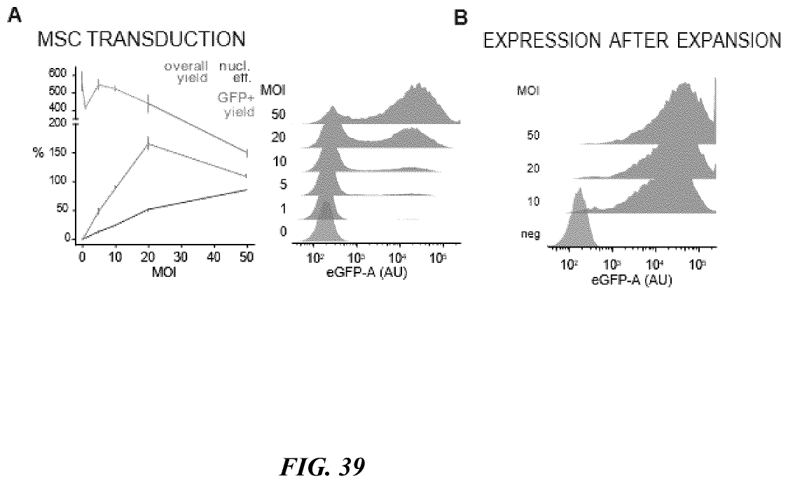

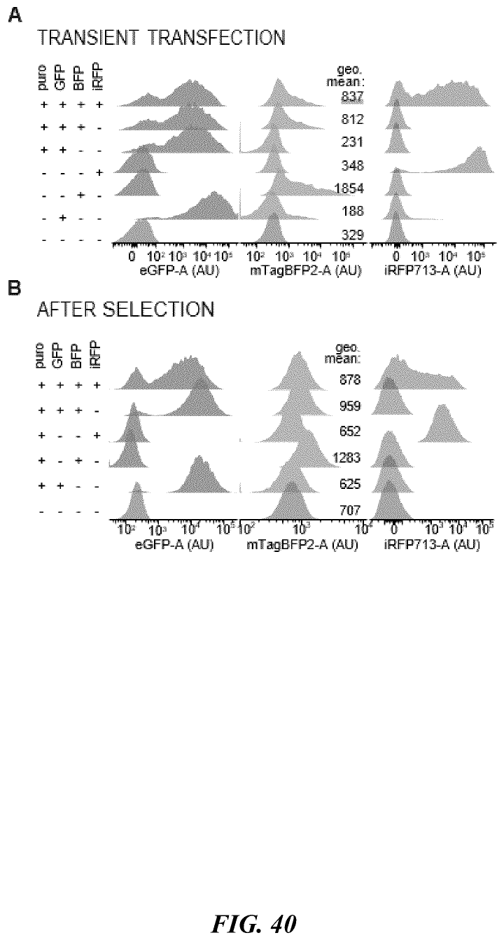

We compared our manufacturing protocol to one that uses lentiviral transduction, which has served as a DNA delivery method for products used in several early-stage clinical trials⁵⁵. We transduced a lentiviral reporter vector using a previously established protocol⁵⁵ that preserved proliferation capabilities⁵⁶, assessing efficiency and yield for cells transduced with various MOIs (FIG. 24A, FIG. 39). We observed the highest transduction efficiency at an MOI of 50, which was lower than our optimized nucleofection protocol but demonstrated a similar GFP+ yield (FIG. 24B, FIG. 40A). After selection and expansion for 3 passages, the lentiviral protocol maintained similar GFP+ yields (FIG. 24C, FIG. 40B) and transgene expression homogeneity as our approach (FIG. 24D). This indicates that our manufacturing protocol can produce similar results to that of lentivirus but has the potential for much larger DNA cargos⁷.

### Impact of DNA Cargo Size on Manufacturability

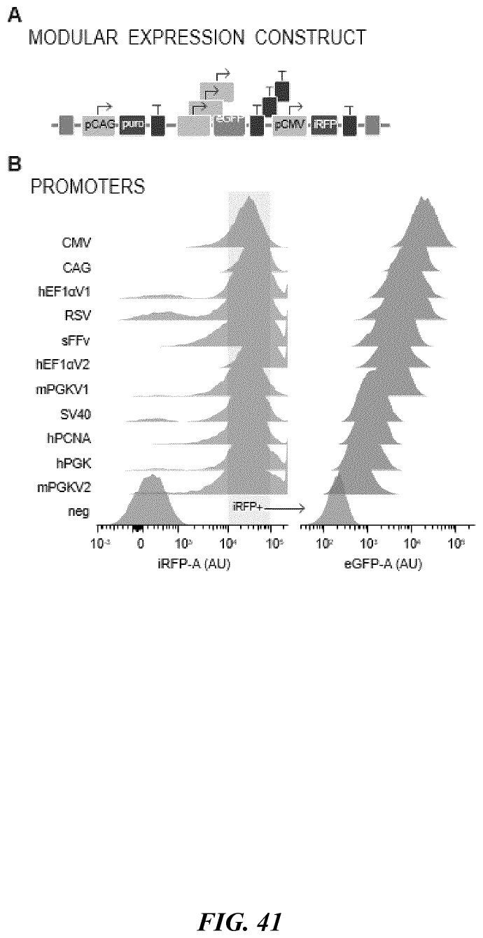

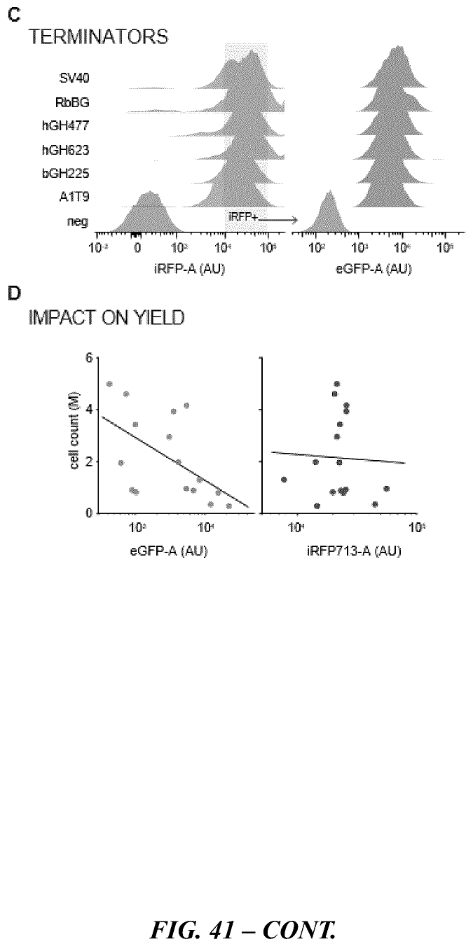

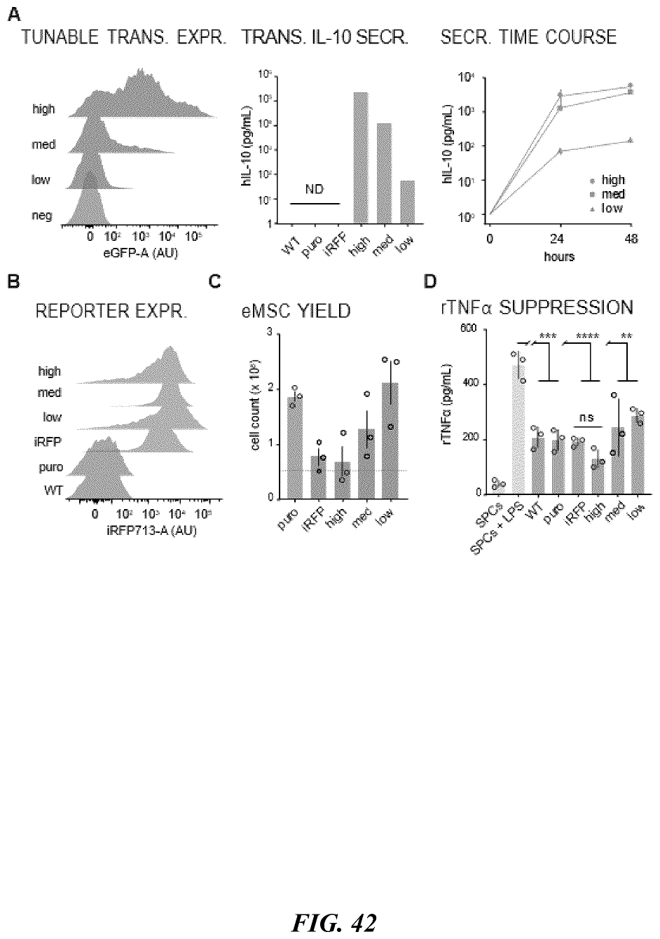

To test whether our protocol supports manufacturing of eMSC products containing large multi-transgene cargos, we built a collection of reporter constructs harboring between 1 and 5 EUs (˜2-12 kb) encoding different combinations and orderings of fluorescent proteins (FIG. 25A). We assessed their impact on transfection, selection, and expansion steps, observing a moderate reduction in nucleofection efficiency as construct size increased, but little reduction in FP+ yield (FIG. 25B, FIG. 41A). We found that the number of cells after selection and 1 passage of expansion decreased as DNA size increased, while expansion kinetics remained constant (FIG. 25C, FIG. 41B). Additionally, swapping promoters or terminators in our reporter (FIG. 42A) and measuring GFP expression allowed us to rank order of expression strength for common constitutive promoters (FIG. 42B) and demonstrate that terminators had little impact on expression (FIG. 42C). We also observed that yield decreased as GFP fluorescence increased (FIG. 42D) demonstrating that, in addition to DNA size, transgene expression level can affect manufacturing.

Engineering eMSCs to Enhance Potency Through In Situ Secretion

To demonstrate that our platform can enhance eMSC functional potency, we engineered multi-transgene cassettes that express human IL-10, a cytokine that has therapeutic potential due to its potent immunosuppressive properties but is not natively expressed by MSCs³². IL-10 has shown limited clinical benefit when delivered exogenously, likely due to its short half-life⁵⁷. Without wishing to be bound by theory, administration of eMSCs engineered to secrete IL-10 is a means to overcome this challenge by allowing in situ secretion in proximity to TNFα producing inflammatory T cells^{1,58} (FIG. 26A, left). We modified our integration construct to encode human IL-10 expressed from one of three promoters with different expression strengths (CMV, high; hEF1a, medium; mPGK, low) (FIGS. 41-42) and iRFP713 was placed at the end of the 3-gene array to monitor product homogeneity and yield⁵⁹ (FIG. 26A, right). Cells harboring each array expanded with similar kinetics (FIG. 26B) and showed homogenous iRFP expression (FIG. 42B). However, consistent with our previous results, iRFP+ yield was inversely correlated with construct size and IL-10 expression level (FIG. 26B, FIG. 42C). When we co-cultured IL-10 expressing eMSCs with LPS-activated rat splenocytes, we found that lower rTNFα suppression was correlated with higher hIL-10 secretion (FIG. 26D, E; FIG. 42D).

### Discussion

Cost effective manufacturing is a primary barrier to widespread clinical use of MSCs, we have developed an efficient, clinical scale, and GMP-compatible manufacturing approach for making eMSCs with complex multi-gene cargos. We can produce >10 billion-cell eMSC products from a small starting population of ˜500 k hUC-MSCs, a >4 log-fold expansion.

Genetic engineering has been used to enhance MSCs, with some making it into the clinic. Examples include agenmestencel-T (MSC_apceth_101, NCT02008539 and 2012-003741-15) 35, an autologous BM-MSC product transduced with gamma retrovirus, and MSCTRAIL (NCT03298763), an allogeneic UC-MSC product transduced with lentivirus no PMID. Non-viral products-include vandefitemcel (SB623, SanBio, NCT02416492) which is uses chemical transfection prior to expansion³¹, and MSC-IFNβ (MD Anderson, NCT02530047) which uses post-expansion nucleofection no PMID. Non-viral approaches can lower costs.

Our approach navigates the intrinsic manufacturing constraints we identified in FIG. 1B and delivers the DNA cargo to a small founding population and then expands them to a clinical scale without exceeding the best practices of clinical expansion.

Our approach is a framework and can be extended to other cell engineering efforts and methods.

The DNA construct (DNA size and expression level) should be considered on how it impacts function and cell product yield. Therefore, the tradeoff between yield and potency should be carefully considered and optimized when designing and manufacturing a clinical product beyond the traditional focus on mechanism of action alone.

Now that we have overcome these manufacturing challenges, we can unlock new opportunities for translating eMSCs to the clinic. By using eMSCs genetically programmed with well-defined, non-native mechanisms of action we can potentially decouple cell product potency from imprecisely defined innate pleiotropic properties.

Finally, we can now use sophisticated genetic engineering and synthetic biology to de-ploy multi-gene cargos that encode sense and respond circuits to user-controlled gene expression of multi-factor secretion.

### Methods

### MSC Harvest

hUC-MSCs were isolated from donated umbilical cords of full-term births without complications after informed consent was obtained from healthy anonymous donors at Memorial Hermann according to a protocol approved by the Institutional Review Board (HSC-MS-11-0601). U1 7204 cells were obtained from the Cord Bank Registry (CBR) at passage 0. U2 4425 and U3 9260 were isolated from donated cords using a modified explant migration method^{53,60,61 30-50} cm cords were collected in PBS (Lonza 17-516F) supplemented with 10 μg/mL gentamycin (Gibco 15710-064). Cords were rinsed in PBS and sterilized with alcohol wipes (Dukal NDC 65517-0002-2). The entire cord was diced using surgical scissors and tissue was distributed into a T225 culture flask (ThermoFisher 156340) pre-coated with 0.1% gelatin (ATCC PCS-999-027) or 5 μg/mL human fibronectin (Gibco 33016-015). After allowing the tissue to adhere for 10 min, 15 mL αMEM (Sigma M4526-500 mL) supplemented with 4.6% human platelet lysate (Sexton PL-NH-100), 1× GlutaMax (Gibco 3505-061), and 10 μg/mL gentamycin (complete αMEM) were added without dislodging the tissue. After 24 h, an additional 15 mL complete αMEM was added. hUC-MSCs migration out of the explant was allowed to proceed for 6 d, after which media was exchanged and cells were expanded for an additional 7 d. On day 14, a cell scraper was used to remove tissue and TrypLE Express (Gibco 12605-010) was used for 8 min at 37° C. to lift the MSCs. Tissue and cells were passed through a 70 μm strainer (Fisherbrand 22363548) and the strained tissue was washed with PBS to dislodge any remaining MSCs. The filtrate containing suspended MSCs was collected, washed with complete αMEM, counted (ChemoMetec NC-200), and frozen in complete αMEM with 5% DMSO as a passage 0 stock as a starting point for future expansion.

AT-MSCs from donors A1 1314, A2 1271, and A3 5713 were isolated in a previous study⁶² and used at passage 0. Briefly, adipose tissue was washed, minced, digested using collagenase, then plated on tissue culture plastic in complete αMEM. BM-MSCs from donors B1 3305, B2 5231, and B3 5190 were isolated in previous work^{63,64} from commercially available fresh human bone marrow aspirates (AllCells BM-FR-50 mL) and used at passage 0. Briefly, mononuclear cells were isolated on a density gradient, plated on tissue culture plastic in complete αMEM. Non-adherent cells were removed at 24 h, and adherent cells were expanded.

### MSC Handling

For all experiments, MSCs and eMSCs were expanded on tissue-culture treated plastic (T75 or T225 flasks, ThermoFisher 156499 and 159934) by plating at 1000-3000 cells/cm² in complete αMEM, incubating at 37° C. and 5% CO₂ with humidity control, and changing media every 2-3 d. For cell manufacturing experiments shown in FIG. 4, eMSCs were expanded using a 5-layer CellSTACK (Corning 3313). Cells were harvested for passage or analysis at 80-90% confluency by aspirating media, washing with PBS, detaching with TrypLE Express (Gibco 12605-010) for 8 min at 37° C., then inactivating the TrpLE with an equal volume of complete αMEM. Cells were collected then centrifuged at 500×g for 8 min, resuspended in complete media, and counted via hemacytometer or cell counter (ChemoMetec NC-200). After each passage, MSCs were frozen in complete αMEM with 5% DMSO (Fisher BioReagents BP231-100) or in CryoStor (BioLife Solutions 210102) as passage 1-3 stocks as starting points for the experiments in this study.

Experiments optimizing nucleofection (FIG. 20), selection and expansion (FIG. 21), lentiviral transduction (FIG. 24), diversifying DNA cargo (FIG. 25), and demonstrating reproducibility (FIG. 34A) used hUC-MSCs from donor U1 7204 at passage 3. Optimized eMSC manufacturing used hUC-MSCs from donor U1 7204 at passage 1 (FIG. 22), while manufacturing hIL-10 producing eMSCs used the same donor at passage 2 (FIG. 26). Experiments evaluating cell source dependence used passage 2 cells from multiple cell sources and donors (FIG. 5). Population doublings were calculated according to the following equation⁶⁷:

$$Pop . doubl . = 3.322 \times log \frac{\# cells harvested}{\# cells plated}$$

$$Doubling time was calculated according to the following {formula}^{66} :$$

$$Doubl . time = \frac{( date harvested - date plated ) \times log2}{log \frac{\# cells harvested}{\# cells plated}} \times 24$$

### Plasmid Construction

Design details of the DNA constructs cloned in this study and the specific experiments they are used for are detailed in Table 5. All constructs were cloned using a custom hierarchical Golden Gate assembly scheme⁴⁴ (FIG. 27). Briefly, input DNA fragments were synthesized de novo or PCR amplified from genomic DNA or commercial DNA sources and cloned into carbenicillin-resistant entry vectors using BsaI-v2 HF (NEB R3733L) to yield genetic part plasmids (e.g., promoters, ORFs, terminators) (level 1). Expression units (EUs) were then constructed by assembling promoter, ORF, and terminator level 1 parts into a kanamycin-resistant entry vector using Esp3I (ThermoFisher ER0452) (level 2). The level 2 entry vector contained mCherry2 for red/white screening and a ccdB negative selection marker, which are both replaced during plasmid assembly. EUs were then combined into arrays of multiple expression units via assembly into a carbenicillin-resistant destination vector using BpiI (ThermoFisher ER1011) (level 3). The final destination vector contains piggyBac transposon inverted terminal repeat (ITR) sequences, a puromycin selectable marker (CAG-puromycin-SV40 pA), BpiI cut sites, mCherry2 for screening, and a ccdB for negative selection. mCherry2 and ccdB markers are replaced by EUs during assembly. All Level 1 parts were confirmed by Sanger Sequencing and plasmid assemblies were verified by nanopore sequencing (Oxford Nanopore Technologies).

TABLE 5
Design details of the DNA constructs cloned in this study.

| | | | | | | Additional |
|---|---|---|---|---|---|---|
| Plasmid # | Parent | EU1 | EU2 | EU3 | EU4 | Information |
| ERN AB | | | | | | Replaced |
| BkB | | | | | | user CDS |
| | | | | | | with ccdB |
| | | | | | | and mCherry2 |
| | | | | | | under |
| | | | | | | bacterial |
| | | | | | | promoters |
| | | | | | | flanked by |
| | | | | | | custom |
| | | | | | | sites for |
| | | | | | | Golden Gate |
| | | | | | | cloning |
| pAJW_{—} | piggyBac | | | | | Replaced |
| MsPBkB16- | backbone | | | | | user CDS |
| 19 AB-AE | VectorBuilder | | | | | with puro, |
| | VB171212- | | | | | installed |
| | 1374ker | | | | | ccdB and |
| | | | | | | mCherry2 |
| | | | | | | under |
| | | | | | | bacterial |
| | | | | | | promoters |
| | | | | | | flanked by |
| | | | | | | custom |
| | | | | | | sites for |
| | | | | | | Golden Gate |
| | | | | | | cloning |
| pOCF_{—} | pHR SIN | | | | | Replaced |
| Ros AB & | backbone | | | | | user CDS |
| AC BkB | pJL048 | | | | | with |
| | | | | | | mCherry2 |
| | | | | | | under |
| | | | | | | bacterial |
| | | | | | | promoters |
| | | | | | | flanked by |
| | | | | | | custom |
| | | | | | | sites for |
| | | | | | | Golden Gate |
| | | | | | | cloning |
| pAJW_E21 | ERN AB | D1-CMV-K100- | | | | n/a |
| | BkB | eGFP-RbBG | | | | |
| pAJW_P26 | pAJW_{—} | D1-CMV-K100- | | | | piggyBac |
| | MsPBkB16 | eGFP-RbBG | | | | ITRs, CAG- |
| | AB | | | | | K100-Puro- |
| | | | | | | SV40 |
| hyPBase | | pRP[Exp]- | | | | VectorBuilder |
| | | mCherry- | | | | VB160216- |
| | | CAG > hyPBase | | | | 10057 |
| pAJW_P26 | pAJW_{—} | D1-CMV-K100- | | | | piggyBac |
| | MsPBkB16 | eGFP-RbBG | | | | ITRs, CAG- |
| | AB | | | | | K100-Puro- |
| | | | | | | SV40 |
| hyPBase | | pRP[Exp]- | | | | VectorBuilder |
| | | mCherry- | | | | VB160216- |
| | | CAG > hyPBase | | | | 10057 |
| pAJW_P26 | pAJW_{—} | D1-CMV-K100- | | | | piggyBac |
| | MsPBkB16 | eGFP-RbBG | | | | ITRs, CAG- |
| | AB | | | | | K100-Puro- |
| | | | | | | SV40 |
| hyPBase | | pRP[Exp]- | | | | VectorBuilder |
| | | mCherry- | | | | VB160216- |
| | | CAG > hyPBase | | | | 10057 |
| pAJW_R3 | pOCF_Ros | eGFP-K100- | hEF1aV1- | | | Lentivirus |
| | AC | CMV-D1 | K100-Puro | | | ITRs |
| psPAX2 | | | | | | Addgene |
| | | | | | | #12260 |
| pMD2.G | | | | | | Addgene |
| | | | | | | #12259 |
| pAJW_E21 | ERN AB | D1-CMV-K100- | | | | n/a |
| | BKB | eGFP-RbBG | | | | |
| pAJW_E30 | ERN AB | D1-CMV-K100- | | | | n/a |
| | BKB | mTagBFP2v3- | | | | |
| | | RbBG | | | | |
| pAJW_E26 | ERN AB | D1-CMV-K100 | | | | n/a |
| | BKB | iRFP713-RbBG | | | | |
| pAJW_E24 | ERN AB | D1-CMV-K100 | | | | n/a |
| | BKB | mCherry-RbBG | | | | |
| pAJW_P26 | pAJW_{—} | D1-CMV-K100- | | | | piggyBac |
| | MsPBkB16 | eGFP-RbBG | | | | ITRs, CAG- |
| | AB | | | | | K100-Puro- |
| | | | | | | SV40 |
| pAJW_P27 | pAJW_{—} | D1-CMV-K100- | | | | piggyBac |
| | MsPBkB16 | mTagBFP2v3- | | | | ITRs, CAG- |
| | AB | RbBG | | | | K100-Puro- |
| | | | | | | SV40 |
| pAJW_P28 | pAJW_{—} | D1-CMV-K100- | | | | piggyBac |
| | MsPBkB16 | iRFP713-RbBG | | | | ITRs, CAG- |
| | AB | | | | | K100-Puro- |
| | | | | | | SV40 |
| pAJW_P29 | pAJW_{—} | D1-CMV-K100- | D1-CMV-K100- | | | piggyBac |
| | MsPBkB17 | eGFP-RbBG | mTagBFP2v3- | | | ITRs, CAG- |
| | AC | | RbBG | | | K100-Puro- |
| | | | | | | SV40 |
| pAJW_P30 | pAJW_{—} | D1-CMV-K100- | D1-CMV-K100- | | | piggyBac |
| | MsPBkB17 | eGFP-RbBG | iRFP713-RbBG | | | ITRs, CAG- |
| | AC | | | | | K100-Puro- |
| | | | | | | SV40 |
| pAJW_P31 | pAJW_{—} | D1-CMV-K100- | D1-CMV-K100- | | | piggyBac |
| | MsPBkB17 | mTagBFP2v3- | iRFP713-RbBG | | | ITRs, CAG- |
| | AC | RbBG | | | | K100-Puro- |
| | | | | | | SV40 |
| pAJW_P32 | pAJW_{—} | D1-CMV-K100- | D1-CMV-K100- | D1-CMV-K100- | | piggyBac |
| | MsPBkB18 | eGFP-RbBG | mTagBFP2v3- | iRFP713-RbBG | | ITRs, CAG- |
| | AD | | RbBG | | | K100-Puro- |
| | | | | | | SV40 |
| pAJW_P33 | pAJW_{—} | D1-CMV-K100- | D1-CMV-K100- | D1-CMV-K100- | D1-CMV-K100- | piggyBac |
| | MsPBkB19 | eGFP-RbBG | mTagBFP2v3- | iRFP713-RbBG | mCherry-RbBG | ITRs, CAG- |
| | AE | | RbBG | | | K100-Puro- |
| | | | | | | SV40 |
| hyPBase | | pRP[Exp]- | | | | VectorBuilder |
| | | mCherry- | | | | VB160216- |
| | | CAG > hyPBase | | | | 10057 |
| pAJW_P48 | pAJW_{—} | CAG-K100- | | | | piggyBac |
| | MsPBkB1 | Puro-SV40 | | | | ITRs |
| | MCS | | | | | |
| pAJW_P28 | pAJW_{—} | D1-CMV-K100- | | | | piggyBac |
| | MsPBkB16 | iRFP713-RbBG | | | | ITRs, CAG- |
| | AB | | | | | K100-Puro- |
| | | | | | | SV40 |
| pAJW_P52 | pAJW_{—} | D1-CMV-K100- | -CMV-K100- | | | piggyBac |
| | MsPBkB17 | hIL-10-hGH477 | iRFP713-RbBG | | | ITRs, CAG- |
| | AC | | | | | K100-Puro- |
| | | | | | | SV40 |
| pAJW_P53 | pAJW_{—} | D1-hEF1aV1- | D1-CMV-K100- | | | piggyBac |
| | MsPBkB17 | K100-hIL-10- | iRFP713-RbBG | | | ITRs, CAG- |
| | AC | hGH477 | | | | K100-Puro- |
| | | | | | | SV40 |
| pAJW_P54 | pAJW_{—} | D1-mPGKsh- | D1-CMV-K100- | | | piggyBac |
| | MsPBkB17 | K100-hIL-10- | iRFP713-RbBG | | | ITRs, CAG- |
| | AC | hGH477 | | | | K100-Puro- |
| | | | | | | SV40 |
| hyPBase | | pRP[Exp]- | | | | VectorBuilder |
| | | mCherry- | | | | VB160216- |
| | | CAG > hyPBase | | | | 10057 |
| pAJW_E21 | ERN AB | D1-CMV-K100- | | | | n/a |
| | BKB | eGFP-RbBG | | | | |
| pAJW_E21 | ERN AB | D1-CMV-K100- | | | | n/a |
| | BKB | eGFP-RbBG | | | | |
| pAJW_E21 | ERN AB | D1-CMV-K100- | | | | n/a |
| | BKB | eGFP-RbBG | | | | |
| pAJW_E21 | ERN AB | D1-CMV-K100- | | | | n/a |
| | BKB | eGFP-RbBG | | | | |
| pAJW_P26 | pAJW_{—} | D1-CMV-K100- | | | | n/a |
| | MsPBkB16 | eGFP-RbBG | | | | |
| | AB | | | | | |
| hyPBase | | pRP[Exp]- | | | | |
| | | mCherry- | | | | |
| | | CAG > hyPBase | | | | |
| pAJW_P26 | pAJW_{—} | D1-CMV-K100- | | | | piggyBac |
| | MsPBkB16 | eGFP-RbBG | | | | ITRs, CAG- |
| | AB | | | | | K100-Puro- |
| | | | | | | SV40 |
| hyPBase | | pRP[Exp]- | | | | VectorBuilder |
| | | mCherry- | | | | VB160216- |
| | | CAG > hyPBase | | | | 10057 |
| pAJW_P26 | pAJW_{—} | D1-CMV-K100- | | | | piggyBac |
| | MsPBkB16 | eGFP-RbBG | | | | ITRs, CAG- |
| | AB | | | | | K100-Puro- |
| | | | | | | SV40 |
| hyPBase | | pRP[Exp]- | | | | VectorBuilder |
| | | mCherry- | | | | VB160216- |
| | | CAG > hyPBase | | | | 10057 |
| pAJW_P26 | pAJW_{—} | D1-CMV-K100- | | | | piggyBac |
| | MsPBkB16 | eGFP-RbBG | | | | ITRs, CAG- |
| | AB | | | | | K100-Puro- |
| | | | | | | SV40 |
| hyPBase | | pRP[Exp]- | | | | VectorBuilder |
| | | mCherry- | | | | VB160216- |
| | | CAG > hyPBase | | | | 10057 |
| pAJW_R1 | pOCF Ros | eGFP-K100- | CAG-K100- | | | Lentivirus |
| | AC | CMV-D1 | Puro | | | ITRs |
| pAJW_R2 | pOCF_Ros | eGFP-K100- | | | | Lentivirus |
| | AB | CMV-D1 | | | | ITRs |
| pAJW_R3 | pOCF Ros | eGFP-K100- | hEF1aV1- | | | Lentivirus |
| | AC | CMV-D1 | K100-Puro | | | ITRs |
| pAJW_R4 | pOCF_Ros | eGFP-K100- | | | | Lentivirus |
| | AB | CAG | | | | ITRs |
| psPAX2 | | | | | | Addgene |
| | | | | | | #12260 |
| pMD2.G | | | | | | Addgene # |
| | | | | | | 12259 |
| pAJW_R3 | pOCF_Ros | eGFP-K100- | hEF1aV1- | | | Lentivirus |
| | AC | CMV-D1 | K100-Puro | | | ITRs |
| psPAX2 | | | | | | Addgene |
| | | | | | | #12260 |
| pMD2.G | | | | | | Addgene |
| | | | | | | #12259 |
| pAJW_E21 | ERN AB | D1-CMV-K100- | | | | n/a |
| | BKB | eGFP-RbBG | | | | |
| pAJW_E30 | ERN AB | D1-CMV-K100- | | | | n/a |
| | BKB | mTagBFP2v3- | | | | |
| | | RbBG | | | | |
| pAJW_E26 | ERN AB | D1-CMV-K100- | | | | n/a |
| | BKB | iRFP713-RbBG | | | | |
| pAJW_E24 | ERN AB | D1-CMV-K100- | | | | n/a |
| | BKB | mCherry-RbBG | | | | |
| pAJW_P26 | pAJW_{—} | D1-CMV-K100- | | | | piggyBac |
| | MsPBkB16 | eGFP-RbBG | | | | ITRs, CAG- |
| | AB | | | | | K100-Puro- |
| | | | | | | SV40 |
| pAJW_P27 | pAJW_{—} | D1-CMV-K100- | | | | piggyBac |
| | MsPBkB16 | mTagBFP2v3- | | | | ITRs, CAG- |
| | AB | RbBG | | | | K100-Puro- |
| | | | | | | SV40 |
| pAJW_P28 | pAJW_{—} | D1-CMV-K100- | | | | piggyBac |
| | MsPBkB16 | iRFP713-RbBG | | | | ITRs, CAG- |
| | AB | | | | | K100-Puro- |
| | | | | | | SV40 |
| pAJW_P29 | pAJW_{—} | D1-CMV-K100- | D1-CMV-K100- | | | piggyBac |
| | MsPBkB17 | eGFP-RbBG | mTagBFP2v3- | | | ITRs, CAG- |
| | AC | | RbBG | | | K100-Puro- |
| | | | | | | SV40 |
| pAJW_P30 | pAJW_{—} | D1-CMV-K100- | D1-CMV-K100- | | | piggyBac |
| | MsPBkB17 | eGFP-RbBG | iRFP713-RbBG | | | ITRs, CAG- |
| | AC | | | | | K100-Puro- |
| | | | | | | SV40 |
| pAJW_P31 | pAJW_{—} | D1-CMV-K100- | D1-CMV-K100- | | | piggyBac |
| | MsPBkB17 | mTagBFP2v3- | iRFP713-RbBG | | | ITRs, CAG- |
| | AC | RbBG | | | | K100-Puro- |
| | | | | | | SV40 |
| pAJW_P32 | pAJW_{—} | D1-CMV-K100- | D1-CMV-K100- | D1-CMV-K100- | | piggyBac |
| | MsPBkB18 | eGFP-RbBG | mTagBFP2v3- | iRFP713-RbBG | | ITRs, CAG- |
| | AD | | RbBG | | | K100-Puro- |
| | | | | | | SV40 |
| hyPBase | | pRP[Exp]- | | | | VectorBuilder |
| | | mCherry- | | | | VB160216- |
| | | CAG > hyPBase | | | | 10057 |
| pAJW_E100 | ERN AB | D1-CMV-K100- | | | | n/a |
| | BKB | eGFP-hGH477 | | | | |
| pAJW_E101 | ERN AB | D1-hEF1aV1- | | | | n/a |
| | BKB | K100-eGFP- | | | | |
| | | hGH477 | | | | |
| pAJW_E102 | ERN AB | D1-mPGKsh- | | | | n/a |
| | BKB | K100-eGFP- | | | | |
| | | hGH477 | | | | |
| pAJW_P48 | pAJW_{—} | CAG-K100- | | | | piggyBac |
| | MsPBkB1 | Puro-SV40 | | | | ITRs |
| | MCS | | | | | |
| pAJW_P28 | pAJW_{—} | D1-CMV-K100- | | | | piggyBac |
| | MsPBkB16 | iRFP713-RbBG | | | | ITRs, CAG- |
| | AB | | | | | K100-Puro- |
| | | | | | | SV40 |
| pAJW_P52 | pAJW_{—} | D1-CMV-K100- | D1-CMV-K100- | | | piggyBac |
| | MsPBkB17 | hIL-10-hGH477 | iRFP713-RbBG | | | ITRs, CAG- |
| | AC | | | | | K100-Puro- |
| | | | | | | SV40 |
| pAJW_P53 | pAJW_{—} | D1-hEF1aV1- | D1-CMV-K100- | | | piggyBac |
| | MsPBkB17 | K100-hIL-10- | iRFP713-RbBG | | | ITRs, CAG- |
| | AC | hGH477 | | | | K100-Puro- |
| | | | | | | SV40 |
| pAJW_P54 | pAJW_{—} | D1-mPGKsh- | D1-CMV-K100- | | | piggyBac |
| | MsPBkB17 | K100-hIL-10- | iRFP713-RbBG | | | ITRs, CAG- |
| | AC | hGH477 | | | | K100-Puro- |
| | | | | | | SV40 |
| hyPBase | | pRP[Exp]- | | | | VectorBuilder |
| | | mCherry- | | | | VB160216- |
| | | CAG > hyPBase | | | | 10057 |
| pAJW_E21 | ERN AB | D1-CMV-K100- | | | | n/a |
| | BKB | eGFP-RbBG | | | | |
| pAJW_P26 | pAJW_{—} | D1-CMV-K100- | | | | piggyBac |
| | MsPBkB16 | eGFP-RbBG | | | | ITRs, CAG- |
| | AB | | | | | K100-Puro- |
| | | | | | | SV40 |
| hyPBase | | pRP[Exp]- | | | | VectorBuilder |
| | | mCherry- | | | | VB160216- |
| | | CAG > hyPBase | | | | 10057 |

TABLE 6
Nucleic acid sequences of DNA constructs cloned in this study.

| DNA Construct | Nucleic Acid Sequence |
|---|---|
| pAJW_MsPBkB-16 | tttttaaccctagaaagatagtctgcgtaaaattgacgcatgcattcttgaaata |
| (AB) | ttgctctctctttctaaatagcgcgaatccgtcgctgtgcatttaggacatctca |
| | gtcgccgcttggagctcccgtgaggcgtgcttgtcaatgcggtaagtgtcactga |
| | ttttgaactataacgaccgcgtgagtcaaaatgacgcatgattatcttttacgtg |
| | acttttaagatttaactcatacgataattatattgttatttcatgttctacttac |
| | gtgataacttattatatatatattttcttgttatagatatcatcaactttgtata |
| | gaaaagttgctcgacattgattattgactagttattaatagtaatcaattacggg |
| | gtcattagttcatagcccatatatggagttccgcgttacataacttacggtaaat |
| | ggcccgcctggctgaccgcccaacgacccccgcccattgacgtcaataatgacgt |
| | atgttcccatagtaacgccaatagggactttccattgacgtcaatgggtggagta |
| | tttacggtaaactgcccacttggcagtacatcaagtgtatcatatgccaagtacg |
| | ccccctattgacgtcaatgacggtaaatggcccgcctggcattatgcccagtaca |
| | tgaccttatgggactttcctacttggcagtacatctacgtattagtcatcgctat |
| | taccatggtcgaggtgagccccacgttctgcttcactctccccatctcccccccc |
| | tccccacccccaattttgtatttatttattttttaattattttgtgcagcgatgg |
| | gggcggggggggggggggggcgcgcgccaggcggggggggcggggcgaggggcgg |
| | ggggggcgaggcggagaggtgcggcggcagccaatcagagcggcgcgctccgaaa |
| | gtttccttttatggcgaggcggcggcggcggcggccctataaaaagcgaagcgcg |
| | cggcggggggagtcgctgcgcgctgccttcgccccgtgccccgctccgccgccgc |
| | ctcgcgccgcccgccccggctctgactgaccgcgttactcccacaggtgagcggg |
| | gggacggcccttctcctccgggctgtaattagcgcttggtttaatgacggcttgt |
| | ttcttttctgtggctgcgtgaaagccttgaggggctccgggagggccctttgtgc |
| | ggggggagcggctcggggggtgcgtgcgtgtgtgtgtgcgtggggagcgccgcgt |
| | gcggctccgcgctgcccggcggctgtgagcgctgcgggcgcggcgcggggctttg |
| | tgcgctccgcagtgtgcgcgaggggagcgcggccgggggcggtgccccgcggtgc |
| | ggggggggctgcgaggggaacaaaggctgcgtgcggggtgtgtgcgtgggggggt |
| | gagcagggggtgtgggcgcgtcggtcgggctgcaaccccccctgcacccccctcc |
| | ccgagttgctgagcacggcccggcttcgggtgcggggctccgtacggggcgtggc |
| | gcggggctcgccgtgccgggcggggggtggcggcaggtgggggtgccgggcgggg |
| | ggggccgcctcgggccggggagggctcgggggaggggcgcggcggcccccggagc |
| | gccggggctgtcgaggcgcggcgagccgcagccattgccttttatggtaatcgtg |
| | cgagagggcgcagggacttcctttgtcccaaatctgtgcggagccgaaatctggg |
| | aggcgccgccgcaccccctctagcgggcgcggggcgaagcggtgcggcgccggca |
| | ggaaggaaatgggcggggagggccttcgtgcgtcgccgcgccgccgtccccttct |
| | ccctctccagcctcggggctgtccgcggggggacggctgccttcgggggggacgg |
| | ggcagggcggggttcggcttctggcgtgtgaccggcggctctagagcctctgcta |
| | accatgttcatgccttcttctttttcctacagctcctgggcaacgtgctggttat |
| | tgtgctgtctcatcattttggcaaagaattgcaagtttgtacaaaaaagcaggct |
| | GAATGCCGCCACCATGGCTaccgagtataaaccgacggtccgacttgcgactcgg |
| | gatgacgttccacgcgccgtgaggactctggccgccgccttcgctgactacccgg |
| | ccacacggcacacagttgaccccgacagacacatagagcgggtcacagagttgca |
| | agaacttttcctgacgcgggttggActcgatattggaaaagtttgggttgccgac |
| | gatggtgccgcagttgcggtgtggactactccagaaagtgtagaagctggagccg |
| | tattcgctgaaatagggccacggatggcagagctgtctggtagcaggctcgcagc |
| | gcagcagcagatggaaggCcttctcgctccgcatcggcctaaggaaccggcgtgg |
| | ttcctggctacggtgggagtctcacctgaccatcaaggaaagggattgggaagtg |
| | ctgtcgttcttccaggcgtggaagcggccgaacgagcaggcgtcccagccttctt |
| | ggaAacgagcgcacccaggaatcttccattctacgagcgactgggtttcactgtg |
| | acagcagacgtagaagtccctgaagggcctcggacttggtgcatgaccagaaagc |
| | ctggggcttgaAACGCCATgatccagacatgataagatacattgatgagtttgga |
| | caaaccacaactagaatgcagtgaGGCGCGCCTGGATACTTCTGCCTGCAGGGGA |
| | GATGTCTTCCAAAttgacggctagctcagtcctaggtacagtgctagcaTACCAG |
| | ATAAGTTCGTCGGTCGGTTATGCAGTTTAAGGTTTACACCTATAAAAGAGAGAGC |
| | CGTTATCGTCTGTTTGTGGATGTACAGAGTGATATTATTGACACGCCCGGGCGAC |
| | GGATGGTGATCCCCCTGGCCAGTGCACGTCTGCTGTCAGATAAAGTCTCCCGTGA |
| | ACTTTACCCGGTGGTGCATATCGGGGATGAAAGCTGGCGCATGATGACCACCGAT |
| | ATGGCCAGTGTGCCGGTGTCCGTTATCGGGGAAGAAGTGGCTGATCTCAGCCACC |
| | GCGAAAATGACATCAAAAACGCCATTAACCTGATGTTCTGGGGAATATAAtaaTG |
| | GCctcggtaccaaattccagaaaagaggcctcccgaaagggggggggggcctttt |
| | ttcgttttggtccgTGGCggaccaaaacgaaaaaagacgcttttcagcgtcttat |
| | tgttcgtctttggtaccgagGCCATTAtttgtatagttcatccatgccaccggta |
| | gaatgacgaccctccgcgcgctcatattgctctacgatcgtataatcttcattat |
| | gagataagatgtccagtttaatatccacattgtacgcgccaggcagctgcacagg |
| | tttcttggctttgtacgtggttttcacttcagcgtcataatggccgccatctttc |
| | agtttcaggcgctgtttaatttcgcctttcagcgcaccatcttccggatacatgc |
| | gttcggtagacgcctcccaacccatcgtcctacactgcatcaccgggccatcaga |
| | tggaaaattagtaccacgcagtttaactttatagatgaactcgccatcctgAagg |
| | gaggagtcctgagtgacggtcacgacaccaccatcttcaaaattcattacgcgtt |
| | cccagttgaaaccttccggaaaagacagcttcagatagtccgggatatccgctgg |
| | gtgtttaacatacgctttagaaccgtacataaattgcgggctcaggatgtcccac |
| | gcaaaaggcagcgggccgcctttagtcactttcagtttggcggtctgggtgcctt |
| | catacggacggccctcgccttcgccttcgatctcgaactcgtgaccgttaacaga |
| | accctccatgtgaactttgaagcgcatgaactctttaatgatagctaagttgttt |
| | tcttctcctttactcacCATTtagtaTTTCTCCTCTTTcactagGTCGagtagct |
| | agcataatacctaggactgagctagctgtaaaGAAGGAAGACATGGCAGCGGCCG |
| | CGCCTGGGCTTACttaattaacgagagcataatattgatatgtgccaaagttgtt |
| | tctgactgactaataagtataatttgtttctattatgtataggttaagctaatta |
| | cttattttataatacaacatgactgtttttaaagtacaaaataagtttatttttg |
| | taaaagagagaatgtttaaaagttttgttactttatagaagaaattttgagtttt |
| | tgtttttttttaataaataaataaacataaataaattgtttgttgaatttattat |
| | tagtatgtaagtgtaaatataataaaacttaatatctattcaaattaataaataa |
| | acctcgatatacagaccgataaaacacatgcgtcaattttacgcatgattatctt |
| | taacgtacgtcacaatatgattatctttctagggttaaataatagtttctaattt |
| | ttttattattcagcctgctgtcgtgaataccgagctccaattcgccctatagtga |
| | gtcgtattacaattcactggccgtcgttttacaacgtcgtgactgggaaaaccct |
| | ggcgttacccaacttaatcgccttgcagcacatccccctttcgccagctggcgta |
| | atagcgaagaggcccgcaccgatcgcccttcccaacagttgcgcagcctgaatgg |
| | cgaatgggacgcgccctgtagcggcgcattaagcgcggcgggtgtggtggttacg |
| | cgcagcgtgaccgctacacttgccagcgccctagcgcccgctcctttcgctttct |
| | tcccttcctttctcgccacgttcgccggctttccccgtcaagctctaaatcgggg |
| | gctccctttagggttccgatttagtgctttacggcacctcgaccccaaaaaactt |
| | gattagggtgatggttcacgtagtgggccatcgccctgatagacggtttttcgcc |
| | ctttgacgttggagtccacgttctttaatagtggactcttgttccaaactggaac |
| | aacactcaaccctatctcggtctattcttttgatttataagggattttgccgatt |
| | tcggcctattggttaaaaaatgagctgatttaacaaaaatttaacgcgaatttta |
| | acaaaatattaacgcttacaatttaggtggcacttttcggggaaatgtgcgcgga |
| | acccctatttgtttatttttctaaatacattcaaatatgtatccgctcatgagac |
| | aataaccctgataaatgcttcaataatattgaaaaaggaagagtatgagtattca |
| | acatttccgtgtcgcccttattcccttttttgcggcattttgccttcctgttttt |
| | gctcacccagaaacgctggtgaaagtaaaagatgctgaagatcagttgggtgcac |
| | gagtgggttacatcgaactggatctcaacagcggtaagatccttgagagttttcg |
| | ccccgaagaacgttttccaatgatgagcacttttaaagttctgctatgtggcgcg |
| | gtattatcccgtattgacgccgggcaagagcaactcggtcgccgcatacactatt |
| | ctcagaatgacttggttgagtactcaccagtcacagaaaagcatcttacggatgg |
| | catgacagtaagagaattatgcagtgctgccataaccatgagtgataacactgcg |
| | gccaacttacttctgacaacgatcggaggaccgaaggagctaaccgcttttttgc |
| | acaacatgggggatcatgtaactcgccttgatcgttgggaaccggagctgaatga |
| | agccataccaaacgacgagcgtgacaccacgatgcctgtagcaatggcaacaacg |
| | ttgcgcaaactattaactggcgaactacttactctagcttcccggcaacaattaa |
| | tagactggatggaggcggataaagttgcaggaccacttctgcgctcggcccttcc |
| | ggctggctggtttattgctgataaatctggagccggtgagcgtgggtctcgcggt |
| | atcattgcagcactggggccagatggtaagccctcccgtatcgtagttatctaca |
| | cgacggggagtcaggcaactatggatgaacgaaatagacagatcgctgagatagg |
| | tgcctcactgattaagcattggtaactgtcagaccaagtttactcatatatactt |
| | tagattgatttaaaacttcatttttaatttaaaaggatctaggtgaagatccttt |
| | ttgataatctcatgaccaaaatcccttaacgtgagttttcgttccactgagcgtc |
| | agaccccgtagaaaagatcaaaggatcttcttgagatcctttttttctgcgcgta |
| | atctgctgcttgcaaacaaaaaaaccaccgctaccagcggtggtttgtttgccgg |
| | atcaagagctaccaactctttttccgaaggtaactggcttcagcagagcgcagat |
| | accaaatactgttcttctagtgtagccgtagttaggccaccacttcaagaactct |
| | gtagcaccgcctacatacctcgctctgctaatcctgttaccagtggctgctgcca |
| | gtggcgataagtcgtgtcttaccgggttggactcaagacgatagttaccggataa |
| | ggcgcagcggtcgggctgaacggggggttcgtgcacacagcccagcttggagcga |
| | acgacctacaccgaactgagatacctacagcgtgagctatgagaaagcgccacgc |
| | ttcccgaagggagaaaggcggacaggtatccggtaagcggcagggtcggaacagg |
| | agagcgcacgagggagcttccagggggaaacgcctggtatctttatagtcctgtc |
| | gggtttcgccacctctgacttgagcgtcgatttttgtgatgctcgtcaggggggc |
| | ggagcctatggaaaaacgccagcaacgcggcctttttacggttcctggccttttg |
| | ctggccttttgctcacatgttctttcctgcgttatcccctgattctgtggataac |
| | cgtattaccgcctttgagtgagctgataccgctcgccgcagccgaacgaccgagc |
| | gcagcgagtcagtgagcgaggaagcggaagagcgcccaatacgcaaaccgcctct |
| | ccccgcgcgttggccgattcattaatgcagctggcacgacaggtttcccgactgg |
| | aaagcgggcagtgagcgcaacgcaattaatgtgagttagctcactcattaggcac |
| | cccaggctttacactttatgcttccggctcgtatgttgtgtggaattgtgagcgg |
| | ataacaatttcacacaggaaacagctatgaccatgattacgccaagctcgaaatt |
| | aaccctcactaaagggaacaaaagctggtacctcgcgcgacttggtttgccattc |
| | tttagcgcgcgtcgcgtcacacagcttggccacaatgtggtttttgtcaaacgaa |
| | gattctatgacgtgtttaaagtttaggtcgagtaaagcgcaaatct |
| | (SEQ ID NO: 7) |
| pAJW_MsPBkB-17 | gtaactgtcagaccaagtttactcatatatactttagattgatttaaaacttcat |
| (AC) | ttttaatttaaaaggatctaggtgaagatcctttttgataatctcatgaccaaaa |
| | tcccttaacgtgagttttcgttccactgagcgtcagaccccgtagaaaagatcaa |
| | aggatcttcttgagatcctttttttctgcgcgtaatctgctgcttgcaaacaaaa |
| | aaaccaccgctaccagcggtggtttgtttgccggatcaagagctaccaactcttt |
| | ttccgaaggtaactggcttcagcagagcgcagataccaaatactgttcttctagt |
| | gtagccgtagttaggccaccacttcaagaactctgtagcaccgcctacatacctc |
| | gctctgctaatcctgttaccagtggctgctgccagtggcgataagtcgtgtctta |
| | ccgggttggactcaagacgatagttaccggataaggcgcagcggtcgggctgaac |
| | ggggggttcgtgcacacagcccagcttggagcgaacgacctacaccgaactgaga |
| | tacctacagcgtgagctatgagaaagcgccacgcttcccgaagggagaaaggcgg |
| | acaggtatccggtaagcggcagggtcggaacaggagagcgcacgagggagcttcc |
| | agggggaaacgcctggtatctttatagtcctgtcgggtttcgccacctctgactt |
| | gagcgtcgatttttgtgatgctcgtcaggggggcggagcctatggaaaaacgcca |
| | gcaacgcggcctttttacggttcctggccttttgctggccttttgctcacatgtt |
| | ctttcctgcgttatcccctgattctgtggataaccgtattaccgcctttgagtga |
| | gctgataccgctcgccgcagccgaacgaccgagcgcagcgagtcagtgagcgagg |
| | aagcggaagagcgcccaatacgcaaaccgcctctccccgcgcgttggccgattca |
| | ttaatgcagctggcacgacaggtttcccgactggaaagcgggcagtgagcgcaac |
| | gcaattaatgtgagttagctcactcattaggcaccccaggctttacactttatgc |
| | ttccggctcgtatgttgtgtggaattgtgagcggataacaatttcacacaggaaa |
| | cagctatgaccatgattacgccaagctcgaaattaaccctcactaaagggaacaa |
| | aagctggtacctcgcgcgacttggtttgccattctttagcgcgcgtcgcgtcaca |
| | cagcttggccacaatgtggtttttgtcaaacgaagattctatgacgtgtttaaag |
| | tttaggtcgagtaaagcgcaaatcttttttaaccctagaaagatagtctgcgtaa |
| | aattgacgcatgcattcttgaaatattgctctctctttctaaatagcgcgaatcc |
| | gtcgctgtgcatttaggacatctcagtcgccgcttggagctcccgtgaggcgtgc |
| | ttgtcaatgcggtaagtgtcactgattttgaactataacgaccgcgtgagtcaaa |
| | atgacgcatgattatcttttacgtgacttttaagatttaactcatacgataatta |
| | tattgttatttcatgttctacttacgtgataacttattatatatatattttcttg |
| | ttatagatatcatcaactttgtatagaaaagttgctcgacattgattattgacta |
| | gttattaatagtaatcaattacggggtcattagttcatagcccatatatggagtt |
| | ccgcgttacataacttacggtaaatggcccgcctggctgaccgcccaacgacccc |
| | cgcccattgacgtcaataatgacgtatgttcccatagtaacgccaatagggactt |
| | tccattgacgtcaatgggtggagtatttacggtaaactgcccacttggcagtaca |
| | tcaagtgtatcatatgccaagtacgccccctattgacgtcaatgacggtaaatgg |
| | cccgcctggcattatgcccagtacatgaccttatgggactttcctacttggcagt |
| | acatctacgtattagtcatcgctattaccatggtcgaggtgagccccacgttctg |
| | cttcactctccccatctcccccccctccccacccccaattttgtatttatttatt |
| | ttttaattattttgtgcagcgatgggggcggggggggggggggggcgcgcgccag |
| | gcggggggggcggggcgaggggcggggcggggcgaggcggagaggtgcggcggca |
| | gccaatcagagcggcgcgctccgaaagtttccttttatggcgaggcggcggcggc |
| | ggcggccctataaaaagcgaagcgcgcggcggggggagtcgctgcgcgctgcctt |
| | cgccccgtgccccgctccgccgccgcctcgcgccgcccgccccggctctgactga |
| | ccgcgttactcccacaggtgagcggggggacggcccttctcctccgggctgtaat |
| | tagcgcttggtttaatgacggcttgtttcttttctgtggctgcgtgaaagccttg |
| | aggggctccgggagggccctttgtgcggggggagcggctcggggggtgcgtgcgt |
| | gtgtgtgtgcgtggggagcgccgcgtgcggctccgcgctgcccggcggctgtgag |
| | cgctgcgggcgcggcgcggggctttgtgcgctccgcagtgtgcgcgaggggagcg |
| | cggccgggggcggtgccccgcggtgcggggggggctgcgaggggaacaaaggctg |
| | cgtgcggggtgtgtgcgtgggggggtgagcagggggtgtgggcgcgtcggtcggg |
| | ctgcaaccccccctgcacccccctccccgagttgctgagcacggcccggcttcgg |
| | gtgcggggctccgtacggggcgtggcgcggggctcgccgtgccgggcggggggtg |
| | gcggcaggtgggggtgccgggcggggcggggccgcctcgggccggggagggctcg |
| | ggggaggggcgcggcggcccccggagcgccggcggctgtcgaggcgcggcgagcc |
| | gcagccattgccttttatggtaatcgtgcgagagggcgcagggacttcctttgtc |
| | ccaaatctgtgcggagccgaaatctgggaggcgccgccgcaccccctctagcggg |
| | cgcggggcgaagcggtgcggcgccggcaggaaggaaatgggcggggagggccttc |
| | gtgcgtcgccgcgccgccgtccccttctccctctccagcctcggggctgtccgcg |
| | gggggacggctgccttcgggggggacggggcagggcggggttcggcttctggcgt |
| | gtgaccggcggctctagagcctctgctaaccatgttcatgccttcttctttttcc |
| | tacagctcctgggcaacgtgctggttattgtgctgtctcatcattttggcaaaga |
| | attgcaagtttgtacaaaaaagcaggctGAATGCCGCCACCATGGCTaccgagta |
| | taaaccgacggtccgacttgcgactcgggatgacgttccacgcgccgtgaggact |
| | ctggccgccgccttcgctgactacccggccacacggcacacagttgaccccgaca |
| | gacacatagagcgggtcacagagttgcaagaacttttcctgacgcgggttggAct |
| | cgatattggaaaagtttgggttgccgacgatggtgccgcagttgcggtgtggact |
| | actccagaaagtgtagaagctggagccgtattcgctgaaatagggccacggatgg |
| | cagagctgtctggtagcaggctcgcagcgcagcagcagatggaaggCcttctcgc |
| | tccgcatcggcctaaggaaccggcgtggttcctggctacggtgggagtctcacct |
| | gaccatcaaggaaagggattgggaagtgctgtcgttcttccaggcgtggaagcgg |
| | ccgaacgagcaggcgtcccagccttcttggaAacgagcgcacccaggaatcttcc |
| | attctacgagcgactgggtttcactgtgacagcagacgtagaagtccctgaaggg |
| | cctcggacttggtgcatgaccagaaagcctggggcttgaAACGCCATgatccaga |
| | catgataagatacattgatgagtttggacaaaccacaactagaatgcagtgaGGC |
| | GCGCCTGGATACTTCTGCCTGCAGGGGAGATGTCTTCCAAAttgacggctagctc |
| | agtcctaggtacagtgctagcaTACCAGATAAGTTCGTCGGTCGGTTATGCAGTT |
| | TAAGGTTTACACCTATAAAAGAGAGAGCCGTTATCGTCTGTTTGTGGATGTACAG |
| | AGTGATATTATTGACACGCCCGGGCGACGGATGGTGATCCCCCTGGCCAGTGCAC |
| | GTCTGCTGTCAGATAAAGTCTCCCGTGAACTTTACCCGGTGGTGCATATCGGGGA |
| | TGAAAGCTGGCGCATGATGACCACCGATATGGCCAGTGTGCCGGTGTCCGTTATC |
| | GGGGAAGAAGTGGCTGATCTCAGCCACCGCGAAAATGACATCAAAAACGCCATTA |
| | ACCTGATGTTCTGGGGAATATAAtaaTGGCctcggtaccaaattccagaaaagag |
| | gcctcccgaaaggggggccttttttcgttttggtccgTGGCggaccaaaacgaaa |
| | aaagacgcttttcagcgtcttattgttcgtctttggtaccgagGCCATTAtttgt |
| | atagttcatccatgccaccggtagaatgacgaccctccgcgcgctcatattgctc |
| | tacgatcgtataatcttcattatgagataagatgtccagtttaatatccacattg |
| | tacgcgccaggcagctgcacaggtttcttggctttgtacgtggttttcacttcag |
| | cgtcataatggccgccatctttcagtttcaggcgctgtttaatttcgcctttcag |
| | cgcaccatcttccggatacatgcgttcggtagacgcctcccaacccatcgtccta |
| | cactgcatcaccgggccatcagatggaaaattagtaccacgcagtttaactttat |
| | agatgaactcgccatcctgAagggaggagtcctgagtgacggtcacgacaccacc |
| | atcttcaaaattcattacgcgttcccagttgaaaccttccggaaaagacagcttc |
| | agatagtccgggatatccgctgggtgtttaacatacgctttagaaccgtacataa |
| | attgcgggctcaggatgtcccacgcaaaaggcagcgggccgcctttagtcacttt |
| | cagtttggcggtctgggtgccttcatacggacggccctcgccttcgccttcgatc |
| | tcgaactcgtgaccgttaacagaaccctccatgtgaactttgaagcgcatgaact |
| | ctttaatgatagctaagttgttttcttctcctttactcacCATTtagtaTTTCTC |
| | CTCTTTcactagGTCGagtagctagcataatacctaggactgagctagctgtaaa |
| | GAAGGAAGACATTCGCGCGGCCGCGCCTGGGCTTACttaattaacgagagcataa |
| | tattgatatgtgccaaagttgtttctgactgactaataagtataatttgtttcta |
| | ttatgtataggttaagctaattacttattttataatacaacatgactgtttttaa |
| | agtacaaaataagtttatttttgtaaaagagagaatgtttaaaagttttgttact |
| | ttatagaagaaattttgagtttttgtttttttttaataaataaataaacataaat |
| | aaattgtttgttgaatttattattagtatgtaagtgtaaatataataaaacttaa |
| | tatctattcaaattaataaataaacctcgatatacagaccgataaaacacatgcg |
| | tcaattttacgcatgattatctttaacgtacgtcacaatatgattatctttctag |
| | ggttaaataatagtttctaatttttttattattcagcctgctgtcgtgaataccg |
| | agctccaattcgccctatagtgagtcgtattacaattcactggccgtcgttttac |
| | aacgtcgtgactgggaaaaccctggcgttacccaacttaatcgccttgcagcaca |
| | tccccctttcgccagctggcgtaatagcgaagaggcccgcaccgatcgcccttcc |
| | caacagttgcgcagcctgaatggcgaatgggacgcgccctgtagcggcgcattaa |
| | gcgcggcgggtgtggtggttacgcgcagcgtgaccgctacacttgccagcgccct |
| | agcgcccgctcctttcgctttcttcccttcctttctcgccacgttcgccggcttt |
| | ccccgtcaagctctaaatcgggggctccctttagggttccgatttagtgctttac |
| | ggcacctcgaccccaaaaaacttgattagggtgatggttcacgtagtgggccatc |
| | gccctgatagacggtttttcgccctttgacgttggagtccacgttctttaatagt |
| | ggactcttgttccaaactggaacaacactcaaccctatctcggtctattcttttg |
| | atttataagggattttgccgatttcggcctattggttaaaaaatgagctgattta |
| | acaaaaatttaacgcgaattttaacaaaatattaacgcttacaatttaggtggca |
| | cttttcggggaaatgtgcgcggaacccctatttgtttatttttctaaatacattc |
| | aaatatgtatccgctcatgagacaataaccctgataaatgcttcaataatattga |
| | aaaaggaagagtatgagtattcaacatttccgtgtcgcccttattcccttttttg |
| | cggcattttgccttcctgtttttgctcacccagaaacgctggtgaaagtaaaaga |
| | tgctgaagatcagttgggtgcacgagtgggttacatcgaactggatctcaacagc |
| | ggtaagatccttgagagttttcgccccgaagaacgttttccaatgatgagcactt |
| | ttaaagttctgctatgtggcgcggtattatcccgtattgacgccgggcaagagca |
| | actcggtcgccgcatacactattctcagaatgacttggttgagtactcaccagtc |
| | acagaaaagcatcttacggatggcatgacagtaagagaattatgcagtgctgcca |
| | taaccatgagtgataacactgcggccaacttacttctgacaacgatcggaggacc |
| | gaaggagctaaccgcttttttgcacaacatgggggatcatgtaactcgccttgat |
| | cgttgggaaccggagctgaatgaagccataccaaacgacgagcgtgacaccacga |
| | tgcctgtagcaatggcaacaacgttgcgcaaactattaactggcgaactacttac |
| | tctagcttcccggcaacaattaatagactggatggaggcggataaagttgcagga |
| | ccacttctgcgctcggcccttccggctggctggtttattgctgataaatctggag |
| | ccggtgagcgtgggtctcgcggtatcattgcagcactggggccagatggtaagcc |
| | ccccgtatcgtagttatctacacgacggggagtcaggcaactatggatgaacgaa |
| | atagacagatcgctgagataggtgcctcactgattaagcattg |
| | (SEQ ID NO: 8) |
| pAJW MsPBkB-18 | cgggcaagagcaactcggtcgccgcatacactattctcagaatgacttggttgag |
| (AD) | tactcaccagtcacagaaaagcatcttacggatggcatgacagtaagagaattat |
| | gcagtgctgccataaccatgagtgataacactgcggccaacttacttctgacaac |
| | gatcggaggaccgaaggagctaaccgcttttttgcacaacatgggggatcatgta |
| | actcgccttgatcgttgggaaccggagctgaatgaagccataccaaacgacgagc |
| | gtgacaccacgatgcctgtagcaatggcaacaacgttgcgcaaactattaactgg |
| | cgaactacttactctagcttcccggcaacaattaatagactggatggaggcggat |
| | aaagttgcaggaccacttctgcgctcggcccttccggctggctggtttattgctg |
| | ataaatctggagccggtgagcgtgggtctcgcggtatcattgcagcactggggcc |
| | agatggtaagccctcccgtatcgtagttatctacacgacggggagtcaggcaact |
| | atggatgaacgaaatagacagatcgctgagataggtgcctcactgattaagcatt |
| | ggtaactgtcagaccaagtttactcatatatactttagattgatttaaaacttca |
| | tttttaatttaaaaggatctaggtgaagatcctttttgataatctcatgaccaaa |
| | atcccttaacgtgagttttcgttccactgagcgtcagaccccgtagaaaagatca |
| | aaggatcttcttgagatcctttttttctgcgcgtaatctgctgcttgcaaacaaa |
| | aaaaccaccgctaccagcggtggtttgtttgccggatcaagagctaccaactctt |
| | tttccgaaggtaactggcttcagcagagcgcagataccaaatactgttcttctag |
| | tgtagccgtagttaggccaccacttcaagaactctgtagcaccgcctacatacct |
| | cgctctgctaatcctgttaccagtggctgctgccagtggcgataagtcgtgtctt |
| | accgggttggactcaagacgatagttaccggataaggcgcagcggtcgggctgaa |
| | cggggggttcgtgcacacagcccagcttggagcgaacgacctacaccgaactgag |
| | atacctacagcgtgagctatgagaaagcgccacgcttcccgaagggagaaaggcg |
| | gacaggtatccggtaagcggcagggtcggaacaggagagcgcacgagggagcttc |
| | cagggggaaacgcctggtatctttatagtcctgtcgggtttcgccacctctgact |
| | tgagcgtcgatttttgtgatgctcgtcaggggggcggagcctatggaaaaacgcc |
| | agcaacgcggcctttttacggttcctggccttttgctggccttttgctcacatgt |
| | tctttcctgcgttatcccctgattctgtggataaccgtattaccgcctttgagtg |
| | agctgataccgctcgccgcagccgaacgaccgagcgcagcgagtcagtgagcgag |
| | gaagcggaagagcgcccaatacgcaaaccgcctctccccgcgcgttggccgattc |
| | attaatgcagctggcacgacaggtttcccgactggaaagcgggcagtgagcgcaa |
| | cgcaattaatgtgagttagctcactcattaggcaccccaggctttacactttatg |
| | cttccggctcgtatgttgtgtggaattgtgagcggataacaatttcacacaggaa |
| | acagctatgaccatgattacgccaagctcgaaattaaccctcactaaagggaaca |
| | aaagctggtacctcgcgcgacttggtttgccattctttagcgcgcgtcgcgtcac |
| | acagcttggccacaatgtggtttttgtcaaacgaagattctatgacgtgtttaaa |
| | gtttaggtcgagtaaagcgcaaatcttttttaaccctagaaagatagtctgcgta |
| | aaattgacgcatgcattcttgaaatattgctctctctttctaaatagcgcgaatc |
| | cgtcgctgtgcatttaggacatctcagtcgccgcttggagctcccgtgaggcgtg |
| | cttgtcaatgcggtaagtgtcactgattttgaactataacgaccgcgtgagtcaa |
| | aatgacgcatgattatcttttacgtgacttttaagatttaactcatacgataatt |
| | atattgttatttcatgttctacttacgtgataacttattatatatatattttctt |
| | gttatagatatcatcaactttgtatagaaaagttgctcgacattgattattgact |
| | agttattaatagtaatcaattacggggtcattagttcatagcccatatatggagt |
| | tccgcgttacataacttacggtaaatggcccgcctggctgaccgcccaacgaccc |
| | ccgcccattgacgtcaataatgacgtatgttcccatagtaacgccaatagggact |
| | ttccattgacgtcaatgggtggagtatttacggtaaactgcccacttggcagtac |
| | atcaagtgtatcatatgccaagtacgccccctattgacgtcaatgacggtaaatg |
| | gcccgcctggcattatgcccagtacatgaccttatgggactttcctacttggcag |
| | tacatctacgtattagtcatcgctattaccatggtcgaggtgagccccacgttct |
| | gcttcactctccccatctcccccccctccccacccccaattttgtatttatttat |
| | tttttaattattttgtgcagcgatgggggcggggggggggggggggcgcgcgcca |
| | ggcggggcggggcggggcgaggggcggggcggggcgaggcggagaggtgcggcgg |
| | cagccaatcagagcggcgcgctccgaaagtttccttttatggcgaggcggcggcg |
| | gcggcggccctataaaaagcgaagcgcgcggcgggcgggagtcgctgcgcgctgc |
| | cttcgccccgtgccccgctccgccgccgcctcgcgccgcccgccccggctctgac |
| | tgaccgcgttactcccacaggtgagcggggggacggcccttctcctccgggctgt |
| | aattagcgcttggtttaatgacggcttgtttcttttctgtggctgcgtgaaagcc |
| | ttgaggggctccgggagggccctttgtgcggggggagcggctcggggggtgcgtg |
| | cgtgtgtgtgtgcgtggggagcgccgcgtgcggctccgcgctgcccggcggctgt |
| | gagcgctgcgggcgcggcgcggggctttgtgcgctccgcagtgtgcgcgagggga |
| | gcgcggccgggggcggtgccccgcggtgcggggggggctgcgaggggaacaaagg |
| | ctgcgtgcggggtgtgtgcgtgggggggtgagcagggggtgtgggcgcgtcggtc |
| | gggctgcaaccccccctgcacccccctccccgagttgctgagcacggcccggctt |
| | cgggtgcggggctccgtacggggcgtggcgcggggctcgccgtgccggggggggg |
| | tggcggcaggtgggggtgccgggcggggggggccgcctcgggccggggagggctc |
| | gggggaggggcgcggcggcccccggagcgccggcggctgtcgaggcgcggcgagc |
| | cgcagccattgccttttatggtaatcgtgcgagagggcgcagggacttcctttgt |
| | cccaaatctgtgcggagccgaaatctgggaggcgccgccgcaccccctctagcgg |
| | gcgcggggcgaagcggtgcggcgccggcaggaaggaaatgggcggggagggcctt |
| | cgtgcgtcgccgcgccgccgtccccttctccctctccagcctcggggctgtccgc |
| | ggggggacggctgccttcgggggggacggggcagggcggggttcggcttctggcg |
| | tgtgaccggcggctctagagcctctgctaaccatgttcatgccttcttctttttc |
| | ctacagctcctgggcaacgtgctggttattgtgctgtctcatcattttggcaaag |
| | aattgcaagtttgtacaaaaaagcaggctGAATGCCGCCACCATGGCTaccgagt |
| | ataaaccgacggtccgacttgcgactcgggatgacgttccacgcgccgtgaggac |
| | tctggccgccgccttcgctgactacccggccacacggcacacagttgaccccgac |
| | agacacatagagcgggtcacagagttgcaagaacttttcctgacgcgggttggAc |
| | tcgatattggaaaagtttgggttgccgacgatggtgccgcagttgcggtgtggac |
| | tactccagaaagtgtagaagctggagccgtattcgctgaaatagggccacggatg |
| | gcagagctgtctggtagcaggctcgcagcgcagcagcagatggaaggCcttctcg |
| | ctccgcatcggcctaaggaaccggcgtggttcctggctacggtgggagtctcacc |
| | tgaccatcaaggaaagggattgggaagtgctgtcgttcttccaggcgtggaagcg |
| | gccgaacgagcaggcgtcccagccttcttggaAacgagcgcacccaggaatcttc |
| | cattctacgagcgactgggtttcactgtgacagcagacgtagaagtccctgaagg |
| | gcctcggacttggtgcatgaccagaaagcctggggcttgaAACGCCATgatccag |
| | acatgataagatacattgatgagtttggacaaaccacaactagaatgcagtgaGG |
| | CGCGCCTGGATACTTCTGCCTGCAGGGGAGATGTCTTCCAAAttgacggctagct |
| | cagtcctaggtacagtgctagcaTACCAGATAAGTTCGTCGGTCGGTTATGCAGT |
| | TTAAGGTTTACACCTATAAAAGAGAGAGCCGTTATCGTCTGTTTGTGGATGTACA |
| | GAGTGATATTATTGACACGCCCGGGCGACGGATGGTGATCCCCCTGGCCAGTGCA |
| | CGTCTGCTGTCAGATAAAGTCTCCCGTGAACTTTACCCGGTGGTGCATATCGGGG |
| | ATGAAAGCTGGCGCATGATGACCACCGATATGGCCAGTGTGCCGGTGTCCGTTAT |
| | CGGGGAAGAAGTGGCTGATCTCAGCCACCGCGAAAATGACATCAAAAACGCCATT |
| | AACCTGATGTTCTGGGGAATATAAtaaTGGCctcggtaccaaattccagaaaaga |
| | ggcctcccgaaaggggggccttttttcgttttggtccgTGGCggaccaaaacgaa |
| | aaaagacgcttttcagcgtcttattgttcgtctttggtaccgagGCCATTAtttg |
| | tatagttcatccatgccaccggtagaatgacgaccctccgcgcgctcatattgct |
| | ctacgatcgtataatcttcattatgagataagatgtccagtttaatatccacatt |
| | gtacgcgccaggcagctgcacaggtttcttggctttgtacgtggttttcacttca |
| | gcgtcataatggccgccatctttcagtttcaggcgctgtttaatttcgcctttca |
| | gcgcaccatcttccggatacatgcgttcggtagacgcctcccaacccatcgtcct |
| | acactgcatcaccgggccatcagatggaaaattagtaccacgcagtttaacttta |
| | tagatgaactcgccatcctgAagggaggagtcctgagtgacggtcacgacaccac |
| | catcttcaaaattcattacgcgttcccagttgaaaccttccggaaaagacagctt |
| | cagatagtccgggatatccgctgggtgtttaacatacgctttagaaccgtacata |
| | aattgcgggctcaggatgtcccacgcaaaaggcagcgggccgcctttagtcactt |
| | tcagtttggcggtctgggtgccttcatacggacggccctcgccttcgccttcgat |
| | ctcgaactcgtgaccgttaacagaaccctccatgtgaactttgaagcgcatgaac |
| | tctttaatgatagctaagttgttttcttctcctttactcacCATTtagtaTTTCT |
| | CCTCTTTcactagGTCGagtagctagcataatacctaggactgagctagctgtaa |
| | aGAAGGAAGACATCAGTGCGGCCGCGCCTGGGCTTACttaattaacgagagcata |
| | atattgatatgtgccaaagttgtttctgactgactaataagtataatttgtttct |
| | attatgtataggttaagctaattacttattttataatacaacatgactgttttta |
| | aagtacaaaataagtttatttttgtaaaagagagaatgtttaaaagttttgttac |
| | tttatagaagaaattttgagtttttgtttttttttaataaataaataaacataaa |
| | taaattgtttgttgaatttattattagtatgtaagtgtaaatataataaaactta |
| | atatctattcaaattaataaataaacctcgatatacagaccgataaaacacatgc |
| | gtcaattttacgcatgattatctttaacgtacgtcacaatatgattatctttcta |
| | gggttaaataatagtttctaatttttttattattcagcctgctgtcgtgaatacc |
| | gagctccaattcgccctatagtgagtcgtattacaattcactggccgtcgtttta |
| | caacgtcgtgactgggaaaaccctggcgttacccaacttaatcgccttgcagcac |
| | atccccctttcgccagctggcgtaatagcgaagaggcccgcaccgatcgcccttc |
| | ccaacagttgcgcagcctgaatggcgaatgggacgcgccctgtagcggcgcatta |
| | agcgcggcgggtgtggtggttacgcgcagcgtgaccgctacacttgccagcgccc |
| | tagcgcccgctcctttcgctttcttcccttcctttctcgccacgttcgccggctt |
| | tccccgtcaagctctaaatcgggggctccctttagggttccgatttagtgcttta |
| | cggcacctcgaccccaaaaaacttgattagggtgatggttcacgtagtgggccat |
| | cgccctgatagacggtttttcgccctttgacgttggagtccacgttctttaatag |
| | tggactcttgttccaaactggaacaacactcaaccctatctcggtctattctttt |
| | gatttataagggattttgccgatttcggcctattggttaaaaaatgagctgattt |
| | aacaaaaatttaacgcgaattttaacaaaatattaacgcttacaatttaggtggc |
| | acttttcggggaaatgtgcgcggaacccctatttgtttatttttctaaatacatt |
| | caaatatgtatccgctcatgagacaataaccctgataaatgcttcaataatattg |
| | aaaaaggaagagtatgagtattcaacatttccgtgtcgcccttattccctttttt |
| | gcggcattttgccttcctgtttttgctcacccagaaacgctggtgaaagtaaaag |
| | atgctgaagatcagttgggtgcacgagtgggttacatcgaactggatctcaacag |
| | cggtaagatccttgagagttttcgccccgaagaacgttttccaatgatgagcact |
| | tttaaagttctgctatgtggcgcggtattatcccgtattgacgc |
| | (SEQ ID NO: 9) |
| pAJW_MsPBkB-19 | ctttacactttatgcttccggctcgtatgttgtgtggaattgtgagcggataaca |
| (AE) | atttcacacaggaaacagctatgaccatgattacgccaagctcgaaattaaccct |
| | cactaaagggaacaaaagctggtacctcgcgcgacttggtttgccattctttagc |
| | gcgcgtcgcgtcacacagcttggccacaatgtggtttttgtcaaacgaagattct |
| | atgacgtgtttaaagtttaggtcgagtaaagcgcaaatcttttttaaccctagaa |
| | agatagtctgcgtaaaattgacgcatgcattcttgaaatattgctctctctttct |
| | aaatagcgcgaatccgtcgctgtgcatttaggacatctcagtcgccgcttggagc |
| | tcccgtgaggcgtgcttgtcaatgcggtaagtgtcactgattttgaactataacg |
| | accgcgtgagtcaaaatgacgcatgattatcttttacgtgacttttaagatttaa |
| | ctcatacgataattatattgttatttcatgttctacttacgtgataacttattat |
| | atatatattttcttgttatagatatcatcaactttgtatagaaaagttgctcgac |
| | attgattattgactagttattaatagtaatcaattacggggtcattagttcatag |
| | cccatatatggagttccgcgttacataacttacggtaaatggcccgcctggctga |
| | ccgcccaacgacccccgcccattgacgtcaataatgacgtatgttcccatagtaa |
| | cgccaatagggactttccattgacgtcaatgggtggagtatttacggtaaactgc |
| | ccacttggcagtacatcaagtgtatcatatgccaagtacgccccctattgacgtc |
| | aatgacggtaaatggcccgcctggcattatgcccagtacatgaccttatgggact |
| | ttcctacttggcagtacatctacgtattagtcatcgctattaccatggtcgaggt |
| | gagccccacgttctgcttcactctccccatctcccccccctccccacccccaatt |
| | ttgtatttatttattttttaattattttgtgcagcgatgggggcggggggggggg |
| | gggggcgcgcgccaggcggggcggggggggcgaggggggggcggggcgaggcgga |
| | gaggtgcggcggcagccaatcagagcggcgcgctccgaaagtttccttttatggc |
| | gaggcggcggcggcggcggccctataaaaagcgaagcgcgcggcggggggagtcg |
| | ctgcgcgctgccttcgccccgtgccccgctccgccgccgcctcgcgccgcccgcc |
| | ccggctctgactgaccgcgttactcccacaggtgagcgggcgggacggcccttct |
| | cctccgggctgtaattagcgcttggtttaatgacggcttgtttcttttctgtggc |
| | tgcgtgaaagccttgaggggctccgggagggccctttgtgcggggggagcggctc |
| | ggggggtgcgtgcgtgtgtgtgtgcgtggggagcgccgcgtgcggctccgcgctg |
| | cccggcggctgtgagcgctgcgggcgcggcgcggggctttgtgcgctccgcagtg |
| | tgcgcgaggggagcgcggccgggggcggtgccccgcggtgcggggggggctgcga |
| | ggggaacaaaggctgcgtgcggggtgtgtgcgtgggggggtgagcagggggtgtg |
| | ggcgcgtcggtcgggctgcaaccccccctgcacccccctccccgagttgctgagc |
| | acggcccggcttcgggtgcggggctccgtacggggcgtggcgcggggctcgccgt |
| | gccgggcggggggggcggcaggtgggggtgccgggcggggggggccgcctcgggc |
| | cggggagggctcgggggaggggcgcggcggcccccggagcgccggcggctgtcga |
| | ggcgcggcgagccgcagccattgccttttatggtaatcgtgcgagagggcgcagg |
| | gacttcctttgtcccaaatctgtgcggagccgaaatctgggaggcgccgccgcac |
| | cccctctagcgggcgcggggcgaagcggtgcggcgccggcaggaaggaaatgggc |
| | ggggagggccttcgtgcgtcgccgcgccgccgtccccttctccctctccagcctc |
| | ggggctgtccgcggggggacggctgccttcgggggggacggggcagggcggggtt |
| | cggcttctggcgtgtgaccggcggctctagagcctctgctaaccatgttcatgcc |
| | ttcttctttttcctacagctcctgggcaacgtgctggttattgtgctgtctcatc |
| | attttggcaaagaattgcaagtttgtacaaaaaagcaggctGAATGCCGCCACCA |
| | TGGCTaccgagtataaaccgacggtccgacttgcgactcgggatgacgttccacg |
| | cgccgtgaggactctggccgccgccttcgctgactacccggccacacggcacaca |
| | gttgaccccgacagacacatagagcgggtcacagagttgcaagaacttttcctga |
| | cgcgggttggActcgatattggaaaagtttgggttgccgacgatggtgccgcagt |
| | tgcggtgtggactactccagaaagtgtagaagctggagccgtattcgctgaaata |
| | gggccacggatggcagagctgtctggtagcaggctcgcagcgcagcagcagatgg |
| | aaggCcttctcgctccgcatcggcctaaggaaccggcgtggttcctggctacggt |
| | gggagtctcacctgaccatcaaggaaagggattgggaagtgctgtcgttcttcca |
| | ggcgtggaagcggccgaacgagcaggcgtcccagccttcttggaAacgagcgcac |
| | ccaggaatcttccattctacgagcgactgggtttcactgtgacagcagacgtaga |
| | agtccctgaagggcctcggacttggtgcatgaccagaaagcctggggcttgaAAC |
| | GCCATgatccagacatgataagatacattgatgagtttggacaaaccacaactag |
| | aatgcagtgaGGCGCGCCTGGATACTTCTGCCTGCAGGGGAGATGTCTTCCAAAt |
| | tgacggctagctcagtcctaggtacagtgctagcaTACCAGATAAGTTCGTCGGT |
| | CGGTTATGCAGTTTAAGGTTTACACCTATAAAAGAGAGAGCCGTTATCGTCTGTT |
| | TGTGGATGTACAGAGTGATATTATTGACACGCCCGGGCGACGGATGGTGATCCCC |
| | CTGGCCAGTGCACGTCTGCTGTCAGATAAAGTCTCCCGTGAACTTTACCCGGTGG |
| | TGCATATCGGGGATGAAAGCTGGCGCATGATGACCACCGATATGGCCAGTGTGCC |
| | GGTGTCCGTTATCGGGGAAGAAGTGGCTGATCTCAGCCACCGCGAAAATGACATC |
| | AAAAACGCCATTAACCTGATGTTCTGGGGAATATAAtaaTGGCctcggtaccaaa |
| | ttccagaaaagaggcctcccgaaaggggggccttttttcgttttggtccgTGGCg |
| | gaccaaaacgaaaaaagacgcttttcagcgtcttattgttcgtctttggtaccga |
| | gGCCATTAtttgtatagttcatccatgccaccggtagaatgacgaccctccgcgc |
| | gctcatattgctctacgatcgtataatcttcattatgagataagatgtccagttt |
| | aatatccacattgtacgcgccaggcagctgcacaggtttcttggctttgtacgtg |
| | gttttcacttcagcgtcataatggccgccatctttcagtttcaggcgctgtttaa |
| | tttcgcctttcagcgcaccatcttccggatacatgcgttcggtagacgcctccca |
| | acccatcgtcctacactgcatcaccgggccatcagatggaaaattagtaccacgc |
| | agtttaactttatagatgaactcgccatcctgAagggaggagtcctgagtgacgg |
| | tcacgacaccaccatcttcaaaattcattacgcgttcccagttgaaaccttccgg |
| | aaaagacagcttcagatagtccgggatatccgctgggtgtttaacatacgcttta |
| | gaaccgtacataaattgcgggctcaggatgtcccacgcaaaaggcagcgggccgc |
| | ctttagtcactttcagtttggcggtctgggtgccttcatacggacggccctcgcc |
| | ttcgccttcgatctcgaactcgtgaccgttaacagaaccctccatgtgaactttg |
| | aagcgcatgaactctttaatgatagctaagttgttttcttctcctttactcacCA |
| | TTtagtaTTTCTCCTCTTTcactagGTCGagtagctagcataatacctaggactg |
| | agctagctgtaaaGAAGGAAGACATTAGGGCGGCCGCGCCTGGGCTTACttaatt |
| | aacgagagcataatattgatatgtgccaaagttgtttctgactgactaataagta |
| | taatttgtttctattatgtataggttaagctaattacttattttataatacaaca |
| | tgactgtttttaaagtacaaaataagtttatttttgtaaaagagagaatgtttaa |
| | aagttttgttactttatagaagaaattttgagtttttgtttttttttaataaata |
| | aataaacataaataaattgtttgttgaatttattattagtatgtaagtgtaaata |
| | taataaaacttaatatctattcaaattaataaataaacctcgatatacagaccga |
| | taaaacacatgcgtcaattttacgcatgattatctttaacgtacgtcacaatatg |
| | attatctttctagggttaaataatagtttctaatttttttattattcagcctgct |
| | gtcgtgaataccgagctccaattcgccctatagtgagtcgtattacaattcactg |
| | gccgtcgttttacaacgtcgtgactgggaaaaccctggcgttacccaacttaatc |
| | gccttgcagcacatccccctttcgccagctggcgtaatagcgaagaggcccgcac |
| | cgatcgcccttcccaacagttgcgcagcctgaatggcgaatgggacgcgccctgt |
| | agcggcgcattaagcgcggcgggtgtggtggttacgcgcagcgtgaccgctacac |
| | ttgccagcgccctagcgcccgctcctttcgctttcttcccttcctttctcgccac |
| | gttcgccggctttccccgtcaagctctaaatcgggggctccctttagggttccga |
| | tttagtgctttacggcacctcgaccccaaaaaacttgattagggtgatggttcac |
| | gtagtgggccatcgccctgatagacggtttttcgccctttgacgttggagtccac |
| | gttctttaatagtggactcttgttccaaactggaacaacactcaaccctatctcg |
| | gtctattcttttgatttataagggattttgccgatttcggcctattggttaaaaa |
| | atgagctgatttaacaaaaatttaacgcgaattttaacaaaatattaacgcttac |
| | aatttaggtggcacttttcggggaaatgtgcgcggaacccctatttgtttatttt |
| | tctaaatacattcaaatatgtatccgctcatgagacaataaccctgataaatgct |
| | tcaataatattgaaaaaggaagagtatgagtattcaacatttccgtgtcgccctt |
| | attcccttttttgcggcattttgccttcctgtttttgctcacccagaaacgctgg |
| | tgaaagtaaaagatgctgaagatcagttgggtgcacgagtgggttacatcgaact |
| | ggatctcaacagcggtaagatccttgagagttttcgccccgaagaacgttttcca |
| | atgatgagcacttttaaagttctgctatgtggcgcggtattatcccgtattgacg |
| | ccgggcaagagcaactcggtcgccgcatacactattctcagaatgacttggttga |
| | gtactcaccagtcacagaaaagcatcttacggatggcatgacagtaagagaatta |
| | tgcagtgctgccataaccatgagtgataacactgcggccaacttacttctgacaa |
| | cgatcggaggaccgaaggagctaaccgcttttttgcacaacatgggggatcatgt |
| | aactcgccttgatcgttgggaaccggagctgaatgaagccataccaaacgacgag |
| | cgtgacaccacgatgcctgtagcaatggcaacaacgttgcgcaaactattaactg |
| | gcgaactacttactctagcttcccggcaacaattaatagactggatggaggcgga |
| | taaagttgcaggaccacttctgcgctcggcccttccggctggctggtttattgct |
| | gataaatctggagccggtgagcgtgggtctcgcggtatcattgcagcactggggc |
| | cagatggtaagccctcccgtatcgtagttatctacacgacggggagtcaggcaac |
| | tatggatgaacgaaatagacagatcgctgagataggtgcctcactgattaagcat |
| | tggtaactgtcagaccaagtttactcatatatactttagattgatttaaaacttc |
| | atttttaatttaaaaggatctaggtgaagatcctttttgataatctcatgaccaa |
| | aatcccttaacgtgagttttcgttccactgagcgtcagaccccgtagaaaagatc |
| | aaaggatcttcttgagatcctttttttctgcgcgtaatctgctgcttgcaaacaa |
| | aaaaaccaccgctaccagcggtggtttgtttgccggatcaagagctaccaactct |
| | ttttccgaaggtaactggcttcagcagagcgcagataccaaatactgttcttcta |
| | gtgtagccgtagttaggccaccacttcaagaactctgtagcaccgcctacatacc |
| | tcgctctgctaatcctgttaccagtggctgctgccagtggcgataagtcgtgtct |
| | taccgggttggactcaagacgatagttaccggataaggcgcagcggtcgggctga |
| | acggggggttcgtgcacacagcccagcttggagcgaacgacctacaccgaactga |
| | gatacctacagcgtgagctatgagaaagcgccacgcttcccgaagggagaaaggc |
| | ggacaggtatccggtaagcggcagggtcggaacaggagagcgcacgagggagctt |
| | ccagggggaaacgcctggtatctttatagtcctgtcgggtttcgccacctctgac |
| | ttgagcgtcgatttttgtgatgctcgtcaggggggcggagcctatggaaaaacgc |
| | cagcaacgcggcctttttacggttcctggccttttgctggccttttgctcacatg |
| | ttctttcctgcgttatcccctgattctgtggataaccgtattaccgcctttgagt |
| | gagctgataccgctcgccgcagccgaacgaccgagcgcagcgagtcagtgagcga |
| | ggaagcggaagagcgcccaatacgcaaaccgcctctccccgcgcgttggccgatt |
| | cattaatgcagctggcacgacaggtttcccgactggaaagcgggcagtgagcgca |
| | acgcaattaatgtgagttagctcactcattaggcaccccagg |
| | (SEQ ID NO: 10) |
| ERN AB BkB | ctgctccctgcttgtgtgttggaggtcgctgagtaggatcctgaagacatGGAGT |
| | CGCtgagacgcaaattgacggctagctcagtcctaggtacagtgctagcatacca |
| | gataagttcgtcggtcggttatgcagtttaaggtttacacctataaaagagagag |
| | ccgttatcgtctgtttgtggatgtacagagtgatattattgacacgcccgggcga |
| | cggatggtgatccccctggccagtgcacgtctgctgtcagataaagtctcccgtg |
| | aactttacccggtggtgcatatcggggatgaaagctggcgcatgatgaacaccga |
| | tatggccagtgtgccggtgtccgttatcggggaagaagtggctgatctcagccac |
| | cgcgaaaatgacatcaaaaacgccattaacctgatgttctggggaatataataat |
| | ggcctcggtaccaaattccagaaaagaggcctcccgaaaggggggccttttttcg |
| | ttttggtccgtggcggaccaaaacgaaaaaagacgcttttcagcgtcttattgtt |
| | cgtctttggtaccgaggccattatttgtatagttcatccatgccaccggtagaat |
| | gacgaccctccgcgcgctcatattgctctacgatcgtataatcttcattatgaga |
| | taagatgtccagtttaatatccacattgtacgcgccaggcagctgcacaggtttc |
| | ttggctttgtacgtggttttcacttcagcgtcataatggccgccatctttcagtt |
| | tcaggcgctgtttaatttcgcctttcagcgcaccatcttccggatacatgcgttc |
| | ggtagacgcctcccaacccatcgtcctacactgcatcaccgggccatcagatgga |
| | aaattagtaccacgcagtttaactttatagatgaactcgccatcctgaagggagg |
| | agtcctgagtgacggtcacgacaccaccatcttcaaaattcattacgcgttccca |
| | gttgaaaccttccggaaaagacagcttcagatagtccgggatatccgctgggtgt |
| | ttaacatacgctttagaaccgtacataaattgcgggctcaggatgtcccacgcaa |
| | aaggcagcgggccgcctttagtcactttcagtttggcggtctgggtgccttcata |
| | cggacggccctcgccttcgccttcgatctcgaactcgtgaccgttaacagaaccc |
| | tccatgtgaactttgaagcgcatgaactctttaatgatagctaagttgttttctt |
| | ctcctttactcaccatttagtatttctcctctttcactaggtcgagtagctagca |
| | taatacctaggactgagctagctgtaaagaagcctgcagggctagcaacgtagcg |
| | tctcaCAGTGGCAatgtcttccggatccgggtcatagctgtttcctgtgtgaaat |
| | tgttatccgctcacaattccacacaacatacgagccggaagcataaagtgtaaag |
| | cctggggtgcctaatgagtgagctaactcacattaattgcgttgcgctcactgcc |
| | cgctttccagtcgggaaacctgtcgtgccagctgcattaatgaatcggccaacgc |
| | gcggggagaggcggtttgcgtattgggcgcacatccgcttcctcgctcactgact |
| | cgctgcgctcggtcgttcggctgcggcgagcggtatcagctcactcaaaggcggt |
| | aatacggttatccacagaatcaggggataacgcaggaaagaacatgtgagcaaaa |
| | ggccagcaaaaggccaggaaccgtaaaaaggccgcgttgctggcgtttttccata |
| | ggctccgcccccctgacgagcatcacaaaaatcgacgctcaagtcagaggtggcg |
| | aaacccgacaggactataaagataccaggcgtttccccctggaagctccctcgtg |
| | cgctctcctgttccgaccctgccgcttacttgatacctgtccgcctttctccctt |
| | cgggaagcgtggcgctttctcatagctcacgctgtaggtatctcagttcggtgta |
| | ggtcgttcgctccaagctgggctgtgtgcacgaaccccccgttcagcccgaccgc |
| | tgcgccttatccggtaactatcgtcttgagtccaacccggtaagacacgacttat |
| | cgccactggcagcagccactggtaacaggattagcagagcgaggtatgtaggcgg |
| | tgctacagagttcttgaagtggtggcctaactacggctacactagaagaacagta |
| | tttggtatctgcgctctgctgaagccagttaccttcggaaaaagagttggtagct |
| | cttgatccggcaaacaaaccaccgctggtagcggtggtttttttgtttgcaagca |
| | gcagattacgcgcagaaaaaaaggatctcaagaagatcctttgatcttttctacg |
| | gggtctgacgctcagtggaacgatgtggtaatgctctgccagtgttacaaccaat |
| | taaccaattctgattagaaaaactcatcgagcatcaaatgaaactgcaatttatt |
| | catatcaggattatcaataccatatttttgaaaaagccgtttctgtaatgaagga |
| | gaaaactcaccgaggcagttccataggatggcaagatcctggtatcggtctgcga |
| | ttccgactcgtccaacatcaatacaacctattaatttcccctcgtcaaaaataag |
| | gttatcaagtgagaaatcaccatgagtgacgactgaatccggtgagaatggcaaa |
| | agtttatgcatttctttccagacttgttcaacaggccagccattacgctcgtcat |
| | caaaatcactcgcatcaaccaaaccgttattcattcgtgattgcgcctgagccag |
| | tcgaaatacgcgatcgctgttaaaaggacaattacaaacaggaatcgaatgcaac |
| | cggcgcaggaacactgccagcgcatcaacaatattttcacctgaatcaggatatt |
| | cttctaatacctggaatgctgtttttccggggatcgcagtggtgagtaaccatgc |
| | atcatcaggagtacggataaaatgcttgatggtcggaagaggcataaattccgtc |
| | agccagtttagtctgaccatctcatctgtaacatcattggcaacgctacctttgc |
| | catgtttcagaaacaactctggcgcatcgggcttcccatacaagcgatagattgt |
| | cgcacctgattgcccgacattatcgcgagcccatttatacccatataaatcagca |
| | tccatgttggaatttaatcgcggcctcgacgtttcccgttgaatatggctcataa |
| | caccccttgtattactgtttatgtaagcagacagttttattgttcatgatgatat |
| | atttttatcttgtgcaatgtaacatcagagattttgagacacggaatcgacgctc |
| | aagtcagaggtcctgacggactcggatcgggagatctcccgatcccctatggtgc |
| | actctcagtacaatctgctctgatgccgcatagttaagccagtat |
| | (SEQ ID NO: 11) |
| hyPBase | caactttgtatagaaaagttgctcgacattgattattgactagttattaatagta |
| | atcaattacggggtcattagttcatagcccatatatggagttccgcgttacataa |
| | cttacggtaaatggcccgcctggctgaccgcccaacgacccccgcccattgacgt |
| | caataatgacgtatgttcccatagtaacgccaatagggactttccattgacgtca |
| | atgggtggagtatttacggtaaactgcccacttggcagtacatcaagtgtatcat |
| | atgccaagtacgccccctattgacgtcaatgacggtaaatggcccgcctggcatt |
| | atgcccagtacatgaccttatgggactttcctacttggcagtacatctacgtatt |
| | agtcatcgctattaccatggtcgaggtgagccccacgttctgcttcactctcccc |
| | atctcccccccctccccacccccaattttgtatttatttattttttaattatttt |
| | gtgcagcgatgggggcggggggggggggggggcgcgcgccaggcggggggggcgg |
| | ggcgaggggggggcggggcgaggcggagaggtgcggcggcagccaatcagagcgg |
| | cgcgctccgaaagtttccttttatggcgaggcggcggcggcggcggccctataaa |
| | aagcgaagcgcgcggggggggagtcgctgcgcgctgccttcgccccgtgccccgc |
| | tccgccgccgcctcgcgccgcccgccccggctctgactgaccgcgttactcccac |
| | aggtgagcgggcgggacggcccttctcctccgggctgtaattagcgcttggttta |
| | atgacggcttgtttcttttctgtggctgcgtgaaagccttgaggggctccgggag |
| | ggccctttgtgcggggggagcggctcggggggtgcgtgcgtgtgtgtgtgcgtgg |
| | ggagcgccgcgtgcggctccgcgctgcccggcggctgtgagcgctgcgggcgcgg |
| | cgcggggctttgtgcgctccgcagtgtgcgcgaggggagcgcggccgggggcggt |
| | gccccgcggtgcggggggggctgcgaggggaacaaaggctgcgtgcggggtgtgt |
| | gcgtgggggggtgagcagggggtgtgggcgcgtcggtcgggctgcaaccccccct |
| | gcacccccctccccgagttgctgagcacggcccggcttcgggtgcggggctccgt |
| | acggggcgtggcgcggggctcgccgtgccgggcggggggggcggcaggtgggggt |
| | gccgggcggggggggccgcctcgggccggggagggctcgggggaggggcgcggcg |
| | gcccccggagcgccggcggctgtcgaggcgcggcgagccgcagccattgcctttt |
| | atggtaatcgtgcgagagggcgcagggacttcctttgtcccaaatctgtgcggag |
| | ccgaaatctgggaggcgccgccgcaccccctctagcgggcgcggggcgaagcggt |
| | gcggcgccggcaggaaggaaatgggcggggagggccttcgtgcgtcgccgcgccg |
| | ccgtccccttctccctctccagcctcggggctgtccgcggggggacggctgcctt |
| | cgggggggacggggcagggcggggttcggcttctggcgtgtgaccggcggctcta |
| | gagcctctgctaaccatgttcatgccttcttctttttcctacagctcctgggcaa |
| | cgtgctggttattgtgctgtctcatcattttggcaaagaattgcaagtttgtaca |
| | aaaaagcaggctgccaccatgggcagcagcctggacgacgagcacatcctgagcg |
| | ccctgctgcagagcgacgacgagctggtcggcgaggacagcgacagcgaggtgag |
| | cgaccacgtgagcgaggacgacgtgcagtccgacaccgaggaggccttcatcgac |
| | gaggtgcacgaggtgcagcctaccagcagcggctccgagatcctggacgagcaga |
| | acgtgatcgagcagcccggcagctccctggccagcaacaggatcctgaccctgcc |
| | ccagaggaccatcaggggcaagaacaagcactgctggtccacctccaagcccacc |
| | aggcggagcagggtgtccgccctgaacatcgtgagaagccagaggggccccacca |
| | ggatgtgcaggaacatctacgaccccctgctgtgcttcaagctgttcttcaccga |
| | cgagatcatcagcgagatcgtgaagtggaccaacgccgagatcagcctgaagagg |
| | cgggagagcatgacctccgccaccttcagggacaccaacgaggacgagatctacg |
| | ccttcttcggcatcctggtgatgaccgccgtgaggaaggacaaccacatgagcac |
| | cgacgacctgttcgacagatccctgagcatggtgtacgtgagcgtgatgagcagg |
| | gacagattcgacttcctgatcagatgcctgaggatggacgacaagagcatcaggc |
| | ccaccctgcgggagaacgacgtgttcacccccgtgagaaagatctgggacctgtt |
| | catccaccagtgcatccagaactacacccctggcgcccacctgaccatcgacgag |
| | cagctgctgggcttcaggggcaggtgccccttcagggtctatatccccaacaagc |
| | ccagcaagtacggcatcaagatcctgatgatgtgcgacagcggcaccaagtacat |
| | gatcaacggcatgccctacctgggcaggggcacccagaccaacggcgtgcccctg |
| | ggcgagtactacgtgaaggagctgtccaagcccgtccacggcagctgcagaaaca |
| | tcacctgcgacaactggttcaccagcatccccctggccaagaacctgctgcagga |
| | gccctacaagctgaccatcgtgggcaccgtgagaagcaacaagagagagatcccc |
| | gaggtcctgaagaacagcaggtccaggcccgtgggcaccagcatgttctgcttcg |
| | acggccccctgaccctggtgtcctacaagcccaagcccgccaagatggtgtacct |
| | gctgtccagctgcgacgaggacgccagcatcaacgagagcaccggcaagccccag |
| | atggtgatgtactacaaccagaccaagggcggcgtggacaccctggaccagatgt |
| | gcagcgtgatgacctgcagcagaaagaccaacaggtggcccatggccctgctgta |
| | cggcatgatcaacatcgcctgcatcaacagcttcatcatctacagccacaacgtg |
| | agcagcaagggcgagaaggtgcagagccggaaaaagttcatgcggaacctgtaca |
| | tgggcctgacctccagcttcatgaggaagaggctggaggcccccaccctgaagag |
| | atacctgagggacaacatcagcaacatcctgcccaaagaggtgcccggcaccagc |
| | gacgacagcaccgaggagcccgtgatgaagaagaggacctactgcacctactgtc |
| | ccagcaagatcagaagaaaggccagcgccagctgcaagaagtgtaagaaggtcat |
| | ctgccgggagcacaacatcgacatgtgccagagctgtttctgaacccagctttct |
| | tgtacaaagtggtgatggccggccgcttcgagcagacatgataagatacattgat |
| | gagtttggacaaaccacaactagaatgcagtgaaaaaaatgctttatttgtgaaa |
| | tttgtgatgctattgctttatttgtaaccattataagctgcaataaacaagttaa |
| | caacaacaattgcattcattttatgtttcaggttcagggggaggtgtgggaggtt |
| | ttttaaagcaagtaaaacctctacaaatgtggtacgcgttgacattgattattga |
| | ctagttattaatagtaatcaattacggggtcattagttcatagcccatatatgga |
| | gttccgcgttacataacttacggtaaatggcccgcctggctgaccgcccaacgac |
| | ccccgcccattgacgtcaataatgacgtatgttcccatagtaacgccaataggga |
| | ctttccattgacgtcaatgggtggagtatttacggtaaactgcccacttggcagt |
| | acatcaagtgtatcatatgccaagtacgccccctattgacgtcaatgacggtaaa |
| | tggcccgcctggcattatgcccagtacatgaccttatgggactttcctacttggc |
| | agtacatctacgtattagtcatcgctattaccatggtgatgcggttttggcagta |
| | catcaatgggcgtggatagcggtttgactcacggggatttccaagtctccacccc |
| | attgacgtcaatgggagtttgttttggcaccaaaatcaacgggactttccaaaat |
| | gtcgtaacaactccgccccattgacgcaaatgggcggtaggcgtgtacggtggga |
| | ggtctatataagcagagctctctggctaactagagaacccactgcgccaccatgg |
| | tgagcaagggcgaggaggataacatggccatcatcaaggagttcatgcgcttcaa |
| | ggtgcacatggagggctccgtgaacggccacgagttcgagatcgagggcgagggc |
| | gagggccgcccctacgagggcacccagaccgccaagctgaaggtgaccaagggtg |
| | gccccctgcccttcgcctgggacatcctgtcccctcagttcatgtacggctccaa |
| | ggcctacgtgaagcaccccgccgacatccccgactacttgaagctgtccttcccc |
| | gagggcttcaagtgggagcgcgtgatgaacttcgaggacggcggcgtggtgaccg |
| | tgacccaggactcctccctgcaggacggcgagttcatctacaaggtgaagctgcg |
| | cggcaccaacttcccctccgacggccccgtaatgcagaagaagaccatgggctgg |
| | gaggcctcctccgagcggatgtaccccgaggacggcgccctgaagggcgagatca |
| | agcagaggctgaagctgaaggacggcggccactacgacgctgaggtcaagaccac |
| | ctacaaggccaagaagcccgtgcagctgcccggcgcctacaacgtcaacatcaag |
| | ttggacatcacctcccacaacgaggactacaccatcgtggaacagtacgaacgcg |
| | ccgagggccgccactccaccggcggcatggacgagctgtacaagtaactcgagtc |
| | tagagggcccgtttaaacccgctgatcagcctcgactgtgccttctagttgccag |
| | ccatctgttgtttgcccctcccccgtgccttccttgaccctggaaggtgccactc |
| | ccactgtcctttcctaataaaatgaggaaattgcatcgcattgtctgagtaggtg |
| | tcattctattctggggggtggggggggcaggacagcaagggggaggattgggaag |
| | acaatagcaggcatgctggggatgcggtgggctctatgggcggccgcggcgctct |
| | tccgcttcctcgctcactgactcgctgcgctcggtcgttcggctgcggcgagcgg |
| | tatcagctcactcaaaggcggtaatacggttatccacagaatcaggggataacgc |
| | aggaaagaacatgtgagcaaaaggccagcaaaaggccaggaaccgtaaaaaggcc |
| | gcgttgctggcgtttttccataggctccgcccccctgacgagcatcacaaaaatc |
| | gacgctcaagtcagaggtggcgaaacccgacaggactataaagataccaggcgtt |
| | tccccctggaagctccctcgtgcgctctcctgttccgaccctgccgcttaccgga |
| | tacctgtccgcctttctcccttcgggaagcgtggcgctttctcatagctcacgct |
| | gtaggtatctcagttcggtgtaggtcgttcgctccaagctgggctgtgtgcacga |
| | accccccgttcagcccgaccgctgcgccttatccggtaactatcgtcttgagtcc |
| | aacccggtaagacacgacttatcgccactggcagcagccactggtaacaggatta |
| | gcagagcgaggtatgtaggcggtgctacagagttcttgaagtggtggcctaacta |
| | cggctacactagaagaacagtatttggtatctgcgctctgctgaagccagttacc |
| | ttcggaaaaagagttggtagctcttgatccggcaaacaaaccaccgctggtagcg |
| | gtggtttttttgtttgcaagcagcagattacgcgcagaaaaaaaggatctcaaga |
| | agatcctttgatcttttctacggggtctgacgctcagtggaacgaaaactcacgt |
| | taagggattttggtcatgagattatcaaaaaggatcttcacctagatccttttaa |
| | attaaaaatgaagttttaaatcaatctaaagtatatatgagtaaacttggtctga |
| | cagttaccaatgcttaatcagtgaggcacctatctcagcgatctgtctatttcgt |
| | tcatccatagttgcctgactccccgtcgtgtagataactacgatacgggagggct |
| | taccatctggccccagtgctgcaatgataccgcgagacccacgctcaccggctcc |
| | agatttatcagcaataaaccagccagccggaagggccgagcgcagaagtggtcct |
| | gcaactttatccgcctccatccagtctattaattgttgccgggaagctagagtaa |
| | gtagttcgccagttaatagtttgcgcaacgttgttgccattgctacaggcatcgt |
| | ggtgtcacgctcgtcgtttggtatggcttcattcagctccggttcccaacgatca |
| | aggcgagttacatgatcccccatgttgtgcaaaaaagcggttagctccttcggtc |
| | ctccgatcgttgtcagaagtaagttggccgcagtgttatcactcatggttatggc |
| | agcactgcataattctcttactgtcatgccatccgtaagatgcttttctgtgact |
| | ggtgagtactcaaccaagtcattctgagaatagtgtatgcggcgaccgagttgct |
| | cttgcccggcgtcaatacgggataataccgcgccacatagcagaactttaaaagt |
| | gctcatcattggaaaacgttcttcggggcgaaaactctcaaggatcttaccgctg |
| | ttgagatccagttcgatgtaacccactcgtgcacccaactgatcttcagcatctt |
| | ttactttcaccagcgtttctgggtgagcaaaaacaggaaggcaaaatgccgcaaa |
| | aaagggaataagggcgacacggaaatgttgaatactcatactcttcctttttcaa |
| | tattattgaagcatttatcagggttattgtctcatgagcggatacatatttgaat |
| | gtatttagaaaaataaacaaataggggttccgcgcacatttccccgaaaagtgcc |
| | acctgacgtctaagaaaccattattatcatgacattaacctataaaaataggcgt |
| | atcacgaggccctttcgtcggcgcgccgcggccgc |
| | (SEQ ID NO: 12) |
| pAJW_E21 | atgaaactgcaatttattcatatcaggattatcaataccatatttttgaaaaagc |
| | cgtttctgtaatgaaggagaaaactcaccgaggcagttccataggatggcaagat |
| | cctggtatcggtctgcgattccgactcgtccaacatcaatacaacctattaattt |
| | cccctcgtcaaaaataaggttatcaagtgagaaatcaccatgagtgacgactgaa |
| | tccggtgagaatggcaaaagtttatgcatttctttccagacttgttcaacaggcc |
| | agccattacgctcgtcatcaaaatcactcgcatcaaccaaaccgttattcattcg |
| | tgattgcgcctgagccagtcgaaatacgcgatcgctgttaaaaggacaattacaa |
| | acaggaatcgaatgcaaccggcgcaggaacactgccagcgcatcaacaatatttt |
| | cacctgaatcaggatattcttctaatacctggaatgctgtttttccggggatcgc |
| | agtggtgagtaaccatgcatcatcaggagtacggataaaatgcttgatggtcgga |
| | agaggcataaattccgtcagccagtttagtctgaccatctcatctgtaacatcat |
| | tggcaacgctacctttgccatgtttcagaaacaactctggcgcatcgggcttccc |
| | atacaagcgatagattgtcgcacctgattgcccgacattatcgcgagcccattta |
| | tacccatataaatcagcatccatgttggaatttaatcgcggcctcgacgtttccc |
| | gttgaatatggctcataacaccccttgtattactgtttatgtaagcagacagttt |
| | tattgttcatgatgatattattttatcttgtgcaatgtaacatcagagattttga |
| | gacacggaatcgacgctcaagtcagaggtcctgacgtcgacggatcgggagatct |
| | cccgatcccctatggtgcactctcagtacaatctgctctgatgccgcatagttaa |
| | gccagtatctgctccctgcttgtgtgttggaggtcgctgagtaGGATCCtGAAGA |
| | CatGGAGTCGCtcttttgcaatgctctttgggaaattatattagcctaattacta |
| | atttcctggccctcaaggtgacttgcttgaacttgccacctTCGCCACTAGGGGG |
| | CAGCAttggtttacacagggtgaacaaacgttcaattctaaactaatattctttg |
| | gtgggaaagtgtgttcattttggtttgttttgaagaagtcacttcatacctttga |
| | actggggttaagctggttaactcccaaaattccaccagttccaatatcctatgga |
| | aaaaccccaaaaccactCAAAccagatatactcgttgacattgattattgactag |
| | ttattaatagtaatcaattacggggtcattagttcatagcccatatatggagttc |
| | cgcgttacataacttacggtaaatggcccgcctggctgaccgcccaacgaccccc |
| | gcccattgacgtcaataatgacgtatgttcccatagtaacgccaatagggacttt |
| | ccattgacgtcaatgggtggagtatttacggtaaactgcccacttggcagtacat |
| | caagtgtatcatatgccaagtacgccccctattgacgtcaatgacggtaaatggc |
| | ccgcctggcattatgcccagtacatgaccttatgggactttcctacttggcagta |
| | catctacgtattagtcatcgctattaccatggtgatgcggttttggcagtacatc |
| | aatgggcgtggatagcggtttgactcacggggatttccaagtctccaccccattg |
| | acgtcaatgggagtttgttttggcaccaaaatcaacgggactttccaaaatgtcg |
| | taacaactccgccccattgacgcaaatgggcggtaggcgtgtacggtgggaggtc |
| | tatataagcagagctctctggctaactagagaacccactgcttactggcttatcg |
| | aaattaatacgactcactatagggagaaccaagcGAATGCCGCCACCATGGCTAT |
| | Ggtgagcaagggcgaggagctgttcaccggggggtgcccatcctggtcgagctgg |
| | acggcgacgtaaacggccacaagttcagcgtgtccggcgagggcgagggcgatgc |
| | cacctacggcaagctgaccctgaagttcatctgcaccaccggcaagctgcccgtg |
| | ccctggcccaccctcgtgaccaccctgacctacggcgtgcagtgcttcagccgct |
| | accccgaccacatgaagcagcacgacttcttcaagtccgccatgcccgaaggcta |
| | cgtccaggagcgcaccatcttcttcaaggacgacggcaactacaagacccgcgcc |
| | gaggtgaagttcgagggcgacaccctggtgaaccgcatcgagctgaagggcatcg |
| | acttcaaggaggacggcaacatcctggggcacaagctggagtacaactacaacag |
| | ccacaacgtctatatcatggccgacaagcagaagaacggcatcaaggtgaacttc |
| | aagatccgccacaacatcgaggacggcagcgtgcagctcgccgaccactaccagc |
| | agaacacccccatcggcgacggccccgtgctgctgcccgacaaccactacctgag |
| | cacccagtccgccctgagcaaagaccccaacgagaagcgcgatcacatggtcctg |
| | ctggagttcgtgaccgccgccgggatcactctcggcatggacgagctgtacaagt |
| | aaAACGCCATgaattcactcctcaggtgcaggctgcctatcagaaggtggtggct |
| | ggtgtggccaatgccctggctcacaaataccactgagatctttttccctctgcca |
| | aaaattatggggacatcatgaagccccttgagcatctgacttctggctaataaag |
| | gaaatttattttcattgcaatagtgtgttggaattttttgtgtctctcactcgga |
| | aggacatatgggagggcaaatcatttaaaacatcagaatgagtatttggtttaga |
| | gtttggcaacatatgcccatatgctggctgccatgaacaaaggttggctataaag |
| | aggtcatcagtatatgaaacagccccctgctgtccattccttattccatagaaaa |
| | gccttgacttgaggttagattttttttatattttgttttgtgttatttttttctt |
| | taacatccctaaaattttccttacatgttttactagccagatttttcctcctctc |
| | ctgactactcccagtcatagctgtccctcttctcttatggagatCAGTGGCAatG |
| | TCTTCcGGATCCgggtcatagctgtttcctgtgtgaaattgttatccgctcacaa |
| | ttccacacaacatacgagccggaagcataaagtgtaaagcctggggtgcctaatg |
| | agtgagctaactcacattaattgcgttgcgctcactgcccgctttccagtcggga |
| | aacctgtcgtgccagctgcattaatgaatcggccaacgcgcggggagaggcggtt |
| | tgcgtattgggcgctcttccgcttcctcgctcactgactcgctgcgctcggtcgt |
| | tcggctgcggcgagcggtatcagctcactcaaaggcggtaatacggttatccaca |
| | gaatcaggggataacgcaggaaagaacatgtgagcaaaaggccagcaaaaggcca |
| | ggaaccgtaaaaaggccgcgttgctggcgtttttccataggctccgcccccctga |
| | cgagcatcacaaaaatcgacgctcaagtcagaggtggcgaaacccgacaggacta |
| | taaagataccaggcgtttccccctggaagctccctcgtgcgctctcctgttccga |
| | ccctgccgcttaccggatacctgtccgcctttctcccttcgggaagcgtggcgct |
| | ttctcatagctcacgctgtaggtatctcagttcggtgtaggtcgttcgctccaag |
| | ctgggctgtgtgcacgaaccccccgttcagcccgaccgctgcgccttatccggta |
| | actatcgtcttgagtccaacccggtaagacacgacttatcgccactggcagcagc |
| | cactggtaacaggattagcagagcgaggtatgtaggcggtgctacagagttcttg |
| | aagtggtggcctaactacggctacactagaagaacagtatttggtatctgcgctc |
| | tgctgaagccagttaccttcggaaaaagagttggtagctcttgatccggcaaaca |
| | aaccaccgctggtagcggtttttttgtttgcaagcagcagattacgcgcagaaaa |
| | aaaggatctcaagaagatcctttgatcttttctacggggtctgacgctcagtgga |
| | acgaatgtggtaatgctctgccagtgttacaaccaattaaccaattctgattaga |
| | aaaactcatcgagcatcaa |
| | (SEQ ID NO: 13) |
| pAJW_E24 | cgtttcccgttgaatatggctcataacaccccttgtattactgtttatgtaagca |
| | gacagttttattgttcatgatgatattattttatcttgtgcaatgtaacatcaga |
| | gattttgagacacggaatcgacgctcaagtcagaggtcctgacgtcgacggatcg |
| | ggagatctcccgatcccctatggtgcactctcagtacaatctgctctgatgccgc |
| | atagttaagccagtatctgctccctgcttgtgtgttggaggtcgctgagtaGGAT |
| | CCtGAAGACatGGAGTCGCtcttttgcaatgctctttgggaaattatattagcct |
| | aattactaatttcctggccctcaaggtgacttgcttgaacttgccacctTCGCCA |
| | CTAGGGGGCAGCAttggtttacacagggtgaacaaacgttcaattctaaactaat |
| | attctttggtgggaaagtgtgttcattttggtttgttttgaagaagtcacttcat |
| | acctttgaactggggttaagctggttaactcccaaaattccaccagttccaatat |
| | cctatggaaaaaccccaaaaccactCAAAccagatatactcgttgacattgatta |
| | ttgactagttattaatagtaatcaattacggggtcattagttcatagcccatata |
| | tggagttccgcgttacataacttacggtaaatggcccgcctggctgaccgcccaa |
| | cgacccccgcccattgacgtcaataatgacgtatgttcccatagtaacgccaata |
| | gggactttccattgacgtcaatgggtggagtatttacggtaaactgcccacttgg |
| | cagtacatcaagtgtatcatatgccaagtacgccccctattgacgtcaatgacgg |
| | taaatggcccgcctggcattatgcccagtacatgaccttatgggactttcctact |
| | tggcagtacatctacgtattagtcatcgctattaccatggtgatgcggttttggc |
| | agtacatcaatgggcgtggatagcggtttgactcacggggatttccaagtctcca |
| | ccccattgacgtcaatgggagtttgttttggcaccaaaatcaacgggactttcca |
| | aaatgtcgtaacaactccgccccattgacgcaaatgggcggtaggcgtgtacggt |
| | gggaggtctatataagcagagctctctggctaactagagaacccactgcttactg |
| | gcttatcgaaattaatacgactcactatagggagaaccaagcGAATGCCGCCACC |
| | ATGGCTggcaatagcgttagcaaaggtgaggaagaTaacatggcgattataaagg |
| | aatttatgcggttcaaggtgcacatggagggttcagttaatggacacgagttcga |
| | aatcgaaggtgagggggaaggtagaccttacgaaggaactcagaccgccaagctg |
| | aaggtgaccaagggtggccccctgcccttcgcctgggacatcctgtcccctcagt |
| | tcatgtacggctccaaggcctacgtgaagcaccccgccgacatccccgactactt |
| | gaagctgtccttccccgagggcttcaagtgggagcgcgtgatgaacttcgaggac |
| | ggcggcgtggtgaccgtgacccaggactcctccttgcaggacggcgagttcatct |
| | acaaggtgaagctgcgcggcaccaacttcccctccgacggccccgtaatgcagaa |
| | gaaaaccatgggctggcaggcctcctccgagcggatgtaccccgaggacggcgcc |
| | ctgaagggcgagatcaagcagaggctgaagctgaaggacggcggccactacgacg |
| | ctgaggtcaagaccacctacaaggccaagaagcccgtgcagctgcccggcgccta |
| | caacgtcaacatcaagttggacatcacctcccacaacgaggactacaccatcgtg |
| | gaacagtacgaacgcgccgagggccgccactccaccggcggcatggacgagctgt |
| | ataagTAAAACGCCATgaattcactcctcaggtgcaggctgcctatcagaaggtg |
| | gtggctggtgtggccaatgccctggctcacaaataccactgagatctttttccct |
| | ctgccaaaaattatggggacatcatgaagccccttgagcatctgacttctggcta |
| | ataaaggaaatttattttcattgcaatagtgtgttggaattttttgtgtctctca |
| | ctcggaaggacatatgggagggcaaatcatttaaaacatcagaatgagtatttgg |
| | tttagagtttggcaacatatgcccatatgctggctgccatgaacaaaggttggct |
| | ataaagaggtcatcagtatatgaaacagccccctgctgtccattccttattccat |
| | agaaaagccttgacttgaggttagattttttttatattttgttttgtgttatttt |
| | tttctttaacatccctaaaattttccttacatgttttactagccagatttttcct |
| | cctctcctgactactcccagtcatagctgtccctcttctcttatggagatCAGTG |
| | GCAatGTCTTCcGGATCCgggtcatagctgtttcctgtgtgaaattgttatccgc |
| | tcacaattccacacaacatacgagccggaagcataaagtgtaaagcctggggtgc |
| | ctaatgagtgagctaactcacattaattgcgttgcgctcactgcccgctttccag |
| | tcgggaaacctgtcgtgccagctgcattaatgaatcggccaacgcgcggggagag |
| | gcggtttgcgtattgggcgctcttccgcttcctcgctcactgactcgctgcgctc |
| | ggtcgttcggctgcggcgagcggtatcagctcactcaaaggcggtaatacggtta |
| | tccacagaatcaggggataacgcaggaaagaacatgtgagcaaaaggccagcaaa |
| | aggccaggaaccgtaaaaaggccgcgttgctggcgtttttccataggctccgccc |
| | ccctgacgagcatcacaaaaatcgacgctcaagtcagaggtggcgaaacccgaca |
| | ggactataaagataccaggcgtttccccctggaagctccctcgtgcgctctcctg |
| | ttccgaccctgccgcttaccggatacctgtccgcctttctcccttcgggaagcgt |
| | ggcgctttctcatagctcacgctgtaggtatctcagttcggtgtaggtcgttcgc |
| | tccaagctgggctgtgtgcacgaaccccccgttcagcccgaccgctgcgccttat |
| | ccggtaactatcgtcttgagtccaacccggtaagacacgacttatcgccactggc |
| | agcagccactggtaacaggattagcagagcgaggtatgtaggcggtgctacagag |
| | ttcttgaagtggtggcctaactacggctacactagaagaacagtatttggtatct |
| | gcgctctgctgaagccagttaccttcggaaaaagagttggtagctcttgatccgg |
| | caaacaaaccaccgctggtagcggtttttttgtttgcaagcagcagattacgcgc |
| | agaaaaaaaggatctcaagaagatcctttgatcttttctacggggtctgacgctc |
| | agtggaacgaatgtggtaatgctctgccagtgttacaaccaattaaccaattctg |
| | attagaaaaactcatcgagcatcaaatgaaactgcaatttattcatatcaggatt |
| | atcaataccatatttttgaaaaagccgtttctgtaatgaaggagaaaactcaccg |
| | aggcagttccataggatggcaagatcctggtatcggtctgcgattccgactcgtc |
| | caacatcaatacaacctattaatttcccctcgtcaaaaataaggttatcaagtga |
| | gaaatcaccatgagtgacgactgaatccggtgagaatggcaaaagtttatgcatt |
| | tctttccagacttgttcaacaggccagccattacgctcgtcatcaaaatcactcg |
| | catcaaccaaaccgttattcattcgtgattgcgcctgagccagtcgaaatacgcg |
| | atcgctgttaaaaggacaattacaaacaggaatcgaatgcaaccggcgcaggaac |
| | actgccagcgcatcaacaatattttcacctgaatcaggatattcttctaatacct |
| | ggaatgctgtttttccggggatcgcagtggtgagtaaccatgcatcatcaggagt |
| | acggataaaatgcttgatggtcggaagaggcataaattccgtcagccagtttagt |
| | ctgaccatctcatctgtaacatcattggcaacgctacctttgccatgtttcagaa |
| | acaactctggcgcatcgggcttcccatacaagcgatagattgtcgcacctgattg |
| | cccgacattatcgcgagcccatttatacccatataaatcagcatccatgttggaa |
| | tttaatcgcggcctcga |
| | (SEQ ID NO: 14) |
| pAJW_E26 | atgaaactgcaatttattcatatcaggattatcaataccatatttttgaaaaagc |
| | cgtttctgtaatgaaggagaaaactcaccgaggcagttccataggatggcaagat |
| | cctggtatcggtctgcgattccgactcgtccaacatcaatacaacctattaattt |
| | cccctcgtcaaaaataaggttatcaagtgagaaatcaccatgagtgacgactgaa |
| | tccggtgagaatggcaaaagtttatgcatttctttccagacttgttcaacaggcc |
| | agccattacgctcgtcatcaaaatcactcgcatcaaccaaaccgttattcattcg |
| | tgattgcgcctgagccagtcgaaatacgcgatcgctgttaaaaggacaattacaa |
| | acaggaatcgaatgcaaccggcgcaggaacactgccagcgcatcaacaatatttt |
| | cacctgaatcaggatattcttctaatacctggaatgctgtttttccggggatcgc |
| | agtggtgagtaaccatgcatcatcaggagtacggataaaatgcttgatggtcgga |
| | agaggcataaattccgtcagccagtttagtctgaccatctcatctgtaacatcat |
| | tggcaacgctacctttgccatgtttcagaaacaactctggcgcatcgggcttccc |
| | atacaagcgatagattgtcgcacctgattgcccgacattatcgcgagcccattta |
| | tacccatataaatcagcatccatgttggaatttaatcgcggcctcgacgtttccc |
| | gttgaatatggctcataacaccccttgtattactgtttatgtaagcagacagttt |
| | tattgttcatgatgatattattttatcttgtgcaatgtaacatcagagattttga |
| | gacacggaatcgacgctcaagtcagaggtcctgacgtcgacggatcgggagatct |
| | cccgatcccctatggtgcactctcagtacaatctgctctgatgccgcatagttaa |
| | gccagtatctgctccctgcttgtgtgttggaggtcgctgagtaGGATCCtGAAGA |
| | CatGGAGTCGCtcttttgcaatgctctttgggaaattatattagcctaattacta |
| | atttcctggccctcaaggtgacttgcttgaacttgccacctTCGCCACTAGGGGG |
| | CAGCAttggtttacacagggtgaacaaacgttcaattctaaactaatattctttg |
| | gtgggaaagtgtgttcattttggtttgttttgaagaagtcacttcatacctttga |
| | actggggttaagctggttaactcccaaaattccaccagttccaatatcctatgga |
| | aaaaccccaaaaccactCAAAccagatatactcgttgacattgattattgactag |
| | ttattaatagtaatcaattacggggtcattagttcatagcccatatatggagttc |
| | cgcgttacataacttacggtaaatggcccgcctggctgaccgcccaacgaccccc |
| | gcccattgacgtcaataatgacgtatgttcccatagtaacgccaatagggacttt |
| | ccattgacgtcaatgggtggagtatttacggtaaactgcccacttggcagtacat |
| | caagtgtatcatatgccaagtacgccccctattgacgtcaatgacggtaaatggc |
| | ccgcctggcattatgcccagtacatgaccttatgggactttcctacttggcagta |
| | catctacgtattagtcatcgctattaccatggtgatgcggttttggcagtacatc |
| | aatgggcgtggatagcggtttgactcacggggatttccaagtctccaccccattg |
| | acgtcaatgggagtttgttttggcaccaaaatcaacgggactttccaaaatgtcg |
| | taacaactccgccccattgacgcaaatgggcggtaggcgtgtacggtgggaggtc |
| | tatataagcagagctctctggctaactagagaacccactgcttactggcttatcg |
| | aaattaatacgactcactatagggagaaccaagcGAATGCCGCCACCATGGCTgc |
| | tgaaggTtccgtcgccaggcagcctgacctcttgacctgcgacgatgagccgatc |
| | catatccccggtgccatccaaccgcatggactgctgctcgccctcgccgccgaca |
| | tgacgatcgttgccggcagcgacaaccttcccgaactcaccggactggcgatcgg |
| | cgccctgatcggccgctctgcggccgatgtGttcgactcggaAacgcacaaccgt |
| | ctgacgatcgccttggccgagcccggggcggccgtcggagcaccgatcactgtcg |
| | gcttcacgatgcgaaaggacgcaggcttcatcggctcctggcatcgccatgatca |
| | gctcatcttcctGgagctGgagcctccccagcgggacgtcgccgagccgcaggcg |
| | ttcttccgccgcaccaacagcgccatccgccgcctgcaggccgccgaaaccttgg |
| | aaagcgcctgcgccgccgcggcgcaagaggtgcggaagattaccggcttcgatcg |
| | ggtgatgatctatcgcttcgcctccgacttcagcggcgaagtgatcgcagaggat |
| | cggtgcgccgaggtcgagtcaaaactaggcctgcactatcctgcctcaaccgtgc |
| | cggcgcaggcccgtcggctctataccatcaacccggtacggatcattcccgatat |
| | caattatcggccggtgccggtcaccccagacctcaatccggtcaccgggcggccg |
| | attgatcttagcttcgccatcctgcgcagcgtGtcgcccgtccatctggaGttca |
| | tgcgcaacataggcatgcacggcacgatgtcgatctcgattttgcgcggcgagcg |
| | actgtggggattgatcgtttgccatcaccgaacgccgtactacgtcgatctcgat |
| | ggccgccaagcctgcgagctagtcgcccaggttctggcctggcagatcggcgtga |
| | tggaagagTAATAGAACGCCATgaattcactcctcaggtgcaggctgcctatcag |
| | aaggtggtggctggtgtggccaatgccctggctcacaaataccactgagatcttt |
| | ttccctctgccaaaaattatggggacatcatgaagccccttgagcatctgacttc |
| | tggctaataaaggaaatttattttcattgcaatagtgtgttggaattttttgtgt |
| | ctctcactcggaaggacatatgggagggcaaatcatttaaaacatcagaatgagt |
| | atttggtttagagtttggcaacatatgcccatatgctggctgccatgaacaaagg |
| | ttggctataaagaggtcatcagtatatgaaacagccccctgctgtccattcctta |
| | ttccatagaaaagccttgacttgaggttagattttttttatattttgttttgtgt |
| | tatttttttctttaacatccctaaaattttccttacatgttttactagccagatt |
| | tttcctcctctcctgactactcccagtcatagctgtccctcttctcttatggaga |
| | tcCAGTGGCAatGTCTTCcGGATCCgggtcatagctgtttcctgtgtgaaattgt |
| | tatccgctcacaattccacacaacatacgagccggaagcataaagtgtaaagcct |
| | ggggtgcctaatgagtgagctaactcacattaattgcgttgcgctcactgcccgc |
| | tttccagtcgggaaacctgtcgtgccagctgcattaatgaatcggccaacgcgcg |
| | gggagaggcggtttgcgtattgggcgctcttccgcttcctcgctcactgactcgc |
| | tgcgctcggtcgttcggctgcggcgagcggtatcagctcactcaaaggcggtaat |
| | acggttatccacagaatcaggggataacgcaggaaagaacatgtgagcaaaaggc |
| | cagcaaaaggccaggaaccgtaaaaaggccgcgttgctggcgtttttccataggc |
| | tccgcccccctgacgagcatcacaaaaatcgacgctcaagtcagaggtggcgaaa |
| | cccgacaggactataaagataccaggcgtttccccctggaagctccctcgtgcgc |
| | tctcctgttccgaccctgccgcttaccggatacctgtccgcctttctcccttcgg |
| | gaagcgtggcgctttctcatagctcacgctgtaggtatctcagttcggtgtaggt |
| | cgttcgctccaagctgggctgtgtgcacgaaccccccgttcagcccgaccgctgc |
| | gccttatccggtaactatcgtcttgagtccaacccggtaagacacgacttatcgc |
| | cactggcagcagccactggtaacaggattagcagagcgaggtatgtaggcggtgc |
| | tacagagttcttgaagtggtggcctaactacggctacactagaagaacagtattt |
| | ggtatctgcgctctgctgaagccagttaccttcggaaaaagagttggtagctctt |
| | gatccggcaaacaaaccaccgctggtagcggtttttttgtttgcaagcagcagat |
| | tacgcgcagaaaaaaaggatctcaagaagatcctttgatcttttctacggggtct |
| | gacgctcagtggaacgaatgtggtaatgctctgccagtgttacaaccaattaacc |
| | aattctgattagaaaaactcatcgagcatcaa |
| | (SEQ ID NO: 15) |
| pAJW_E30 | tttttgaaaaagccgtttctgtaatgaaggagaaaactcaccgaggcagttccat |
| | aggatggcaagatcctggtatcggtctgcgattccgactcgtccaacatcaatac |
| | aacctattaatttcccctcgtcaaaaataaggttatcaagtgagaaatcaccatg |
| | agtgacgactgaatccggtgagaatggcaaaagtttatgcatttctttccagact |
| | tgttcaacaggccagccattacgctcgtcatcaaaatcactcgcatcaaccaaac |
| | cgttattcattcgtgattgcgcctgagccagtcgaaatacgcgatcgctgttaaa |
| | aggacaattacaaacaggaatcgaatgcaaccggcgcaggaacactgccagcgca |
| | tcaacaatattttcacctgaatcaggatattcttctaatacctggaatgctgttt |
| | ttccggggatcgcagtggtgagtaaccatgcatcatcaggagtacggataaaatg |
| | cttgatggtcggaagaggcataaattccgtcagccagtttagtctgaccatctca |
| | tctgtaacatcattggcaacgctacctttgccatgtttcagaaacaactctggcg |
| | catcgggcttcccatacaagcgatagattgtcgcacctgattgcccgacattatc |
| | gcgagcccatttatacccatataaatcagcatccatgttggaatttaatcgcggc |
| | ctcgacgtttcccgttgaatatggctcataacaccccttgtattactgtttatgt |
| | aagcagacagttttattgttcatgatgatattattttatcttgtgcaatgtaaca |
| | tcagagattttgagacacggaatcgacgctcaagtcagaggtcctgacgtcgacg |
| | gatcgggagatctcccgatcccctatggtgcactctcagtacaatctgctctgat |
| | gccgcatagttaagccagtatctgctccctgcttgtgtgttggaggtcgctgagt |
| | aGGATCCtGAAGACatGGAGTCGCtcttttgcaatgctctttgggaaattatatt |
| | agcctaattactaatttcctggccctcaaggtgacttgcttgaacttgccacctT |
| | CGCCACTAGGGGGCAGCAttggtttacacagggtgaacaaacgttcaattctaaa |
| | ctaatattctttggtgggaaagtgtgttcattttggtttgttttgaagaagtcac |
| | ttcatacctttgaactggggttaagctggttaactcccaaaattccaccagttcc |
| | aatatcctatggaaaaaccccaaaaccactCAAAccagatatactcgttgacatt |
| | gattattgactagttattaatagtaatcaattacggggtcattagttcatagccc |
| | atatatggagttccgcgttacataacttacggtaaatggcccgcctggctgaccg |
| | cccaacgacccccgcccattgacgtcaataatgacgtatgttcccatagtaacgc |
| | caatagggactttccattgacgtcaatgggtggagtatttacggtaaactgccca |
| | cttggcagtacatcaagtgtatcatatgccaagtacgccccctattgacgtcaat |
| | gacggtaaatggcccgcctggcattatgcccagtacatgaccttatgggactttc |
| | ctacttggcagtacatctacgtattagtcatcgctattaccatggtgatgcggtt |
| | ttggcagtacatcaatgggcgtggatagcggtttgactcacggggatttccaagt |
| | ctccaccccattgacgtcaatgggagtttgttttggcaccaaaatcaacgggact |
| | ttccaaaatgtcgtaacaactccgccccattgacgcaaatgggcggtaggcgtgt |
| | acggtgggaggtctatataagcagagctctctggctaactagagaacccactgct |
| | tactggcttatcgaaattaatacgactcactatagggagaaccaagcGAATGCCG |
| | CCACCATGGCTGTCAGTAAAGGGGAAgagcttataaaggaaaatatgcacatgaa |
| | gctctacatggagggcactgtagataaccaccatttcaaatgtacctctgaaggg |
| | gagggcaagccatacgaaggtactcaaaccatgcgaataaaagtagttgaaggcg |
| | ggcctcttccctttgcattcgacattctcgcaacctcttttctgtacggcagtaa |
| | gactttcataaaccacactcaaggcattccagacttcttcaagcaatcattcccc |
| | gaaggattcacctgggagcgagttactacttatgaggacggaggagtccttactg |
| | caacccaagacacctcactgcaagatgggtgcctgatttacaatgtaaagatcag |
| | aggggtgaatttcacaagcaatgggccagttatgcaaaaaaagacccttggatgg |
| | gaggccttcaccgagacactgtacccagccgatggtggactggagggcaggaatg |
| | acatggccctcaagctcgtcggaggcagtcacttgattgccaacgccaaaaccac |
| | ttaccgctctaagaaacctgctaaaaacctgaagatgcccggcgtctattatgtg |
| | gactatcgacttgagagaattaaggaggcaaacaacgagacttatgtcgaacagc |
| | atgaagttgccgtggctaggtactgtgatttgcccagcaaattgggtcataaact |
| | taacTGAAACGCCATgaattcactcctcaggtgcaggctgcctatcagaaggtgg |
| | tggctggtgtggccaatgccctggctcacaaataccactgagatctttttccctc |
| | tgccaaaaattatggggacatcatgaagccccttgagcatctgacttctggctaa |
| | taaaggaaatttattttcattgcaatagtgtgttggaattttttgtgtctctcac |
| | tcggaaggacatatgggagggcaaatcatttaaaacatcagaatgagtatttggt |
| | ttagagtttggcaacatatgcccatatgctggctgccatgaacaaaggttggcta |
| | taaagaggtcatcagtatatgaaacagccccctgctgtccattccttattccata |
| | gaaaagccttgacttgaggttagattttttttatattttgttttgtgttattttt |
| | ttctttaacatccctaaaattttccttacatgttttactagccagatttttcctc |
| | ctctcctgactactcccagtcatagctgtccctcttctcttatggagatcCAGTG |
| | GCAatGTCTTCcGGATCCgggtcatagctgtttcctgtgtgaaattgttatccgc |
| | tcacaattccacacaacatacgagccggaagcataaagtgtaaagcctggggtgc |
| | ctaatgagtgagctaactcacattaattgcgttgcgctcactgcccgctttccag |
| | tcgggaaacctgtcgtgccagctgcattaatgaatcggccaacgcgcggggagag |
| | gcggtttgcgtattgggcgctcttccgcttcctcgctcactgactcgctgcgctc |
| | ggtcgttcggctgcggcgagcggtatcagctcactcaaaggcggtaatacggtta |
| | tccacagaatcaggggataacgcaggaaagaacatgtgagcaaaaggccagcaaa |
| | aggccaggaaccgtaaaaaggccgcgttgctggcgtttttccataggctccgccc |
| | ccctgacgagcatcacaaaaatcgacgctcaagtcagaggtggcgaaacccgaca |
| | ggactataaagataccaggcgtttccccctggaagctccctcgtgcgctctcctg |
| | ttccgaccctgccgcttaccggatacctgtccgcctttctcccttcgggaagcgt |
| | ggcgctttctcatagctcacgctgtaggtatctcagttcggtgtaggtcgttcgc |
| | tccaagctgggctgtgtgcacgaaccccccgttcagcccgaccgctgcgccttat |
| | ccggtaactatcgtcttgagtccaacccggtaagacacgacttatcgccactggc |
| | agcagccactggtaacaggattagcagagcgaggtatgtaggcggtgctacagag |
| | ttcttgaagtggtggcctaactacggctacactagaagaacagtatttggtatct |
| | gcgctctgctgaagccagttaccttcggaaaaagagttggtagctcttgatccgg |
| | caaacaaaccaccgctggtagcggtttttttgtttgcaagcagcagattacgcgc |
| | agaaaaaaaggatctcaagaagatcctttgatcttttctacggggtctgacgctc |
| | agtggaacgaatgtggtaatgctctgccagtgttacaaccaattaaccaattctg |
| | attagaaaaactcatcgagcatcaaatgaaactgcaatttattcatatcaggatt |
| | atcaataccata |
| | (SEQ ID NO: 16) |
| pAJW_E100 | cgtttcccgttgaatatggctcataacaccccttgtattactgtttatgtaagca |
| | gacagttttattgttcatgatgatattattttatcttgtgcaatgtaacatcaga |
| | gattttgagacacggaatcgacgctcaagtcagaggtcctgacgtcgacggatcg |
| | ggagatctcccgatcccctatggtgcactctcagtacaatctgctctgatgccgc |
| | atagttaagccagtatctgctccctgcttgtgtgttggaggtcgctgagtaGGAT |
| | CCtGAAGACatGGAGTCGCtcttttgcaatgctctttgggaaattatattagcct |
| | aattactaatttcctggccctcaaggtgacttgcttgaacttgccacctTCGCCA |
| | CTAGGGGGCAGCAttggtttacacagggtgaacaaacgttcaattctaaactaat |
| | attctttggtgggaaagtgtgttcattttggtttgttttgaagaagtcacttcat |
| | acctttgaactggggttaagctggttaactcccaaaattccaccagttccaatat |
| | cctatggaaaaaccccaaaaccactCAAAccagatatactcgttgacattgatta |
| | ttgactagttattaatagtaatcaattacggggtcattagttcatagcccatata |
| | tggagttccgcgttacataacttacggtaaatggcccgcctggctgaccgcccaa |
| | cgacccccgcccattgacgtcaataatgacgtatgttcccatagtaacgccaata |
| | gggactttccattgacgtcaatgggtggagtatttacggtaaactgcccacttgg |
| | cagtacatcaagtgtatcatatgccaagtacgccccctattgacgtcaatgacgg |
| | taaatggcccgcctggcattatgcccagtacatgaccttatgggactttcctact |
| | tggcagtacatctacgtattagtcatcgctattaccatggtgatgcggttttggc |
| | agtacatcaatgggcgtggatagcggtttgactcacggggatttccaagtctcca |
| | ccccattgacgtcaatgggagtttgttttggcaccaaaatcaacgggactttcca |
| | aaatgtcgtaacaactccgccccattgacgcaaatgggcggtaggcgtgtacggt |
| | gggaggtctatataagcagagctctctggctaactagagaacccactgcttactg |
| | gcttatcgaaattaatacgactcactatagggagaaccaagcGAATGCCGCCACC |
| | ATGgtgagcaagggcgaggagctgttcaccggggtggtgcccatcctggtcgagc |
| | tggacggcgacgtaaacggccacaagttcagcgtgtccggcgagggcgagggcga |
| | tgccacctacggcaagctgaccctgaagttcatctgcaccaccggcaagctgccc |
| | gtgccctggcccaccctcgtgaccaccctgacctacggcgtgcagtgcttcagcc |
| | gctaccccgaccacatgaagcagcacgacttcttcaagtccgccatgcccgaagg |
| | ctacgtccaggagcgcaccatcttcttcaaggacgacggcaactacaagacccgc |
| | gccgaggtgaagttcgagggcgacaccctggtgaaccgcatcgagctgaagggca |
| | tcgacttcaaggaggacggcaacatcctggggcacaagctggagtacaactacaa |
| | cagccacaacgtctatatcatggccgacaagcagaagaacggcatcaaggtgaac |
| | ttcaagatccgccacaacatcgaggacggcagcgtgcagctcgccgaccactacc |
| | agcagaacacccccatcggcgacggccccgtgctgctgcccgacaaccactacct |
| | gagcacccagtccgccctgagcaaagaccccaacgagaagcgcgatcacatggtc |
| | ctgctggagttcgtgaccgccgccgggatcactctcggcatggacgagctgtaca |
| | agtaaAACGCCATgggtggcatccctgtgacccctccccagtgcctctcctggcc |
| | ctggaagttgccactccagtgcccaccagccttgtcctaataaaattaagttgca |
| | tcattttgtctgactaggtgtccttctataatattatggggtggaggggggtggt |
| | atggagcaaggggcaagttgggaagagaacctgtagggcctgcggggtctattgg |
| | gaaccaagctggagtgcagtggcacaatcttggctcactgcaatctccgcctcct |
| | gggttcaagcgattctcctgcctcagcctcccgagttgttgggattccaggcatg |
| | catgaccaggctcagctaatttttgtttttttggtagagTcggggtttcaccata |
| | ttggccaggctggtgtccaactcctaatctcaggtgatctacccaccttggcctc |
| | ccaaattgctgggattacaggcgtgaaccactgctcccttccctgtccttCAGTG |
| | GCAatGTCTTCcGGATCCgggtcatagctgtttcctgtgtgaaattgttatccgc |
| | tcacaattccacacaacatacgagccggaagcataaagtgtaaagcctggggtgc |
| | ctaatgagtgagctaactcacattaattgcgttgcgctcactgcccgctttccag |
| | tcgggaaacctgtcgtgccagctgcattaatgaatcggccaacgcgcggggagag |
| | gcggtttgcgtattgggcgctcttccgcttcctcgctcactgactcgctgcgctc |
| | ggtcgttcggctgcggcgagcggtatcagctcactcaaaggcggtaatacggtta |
| | tccacagaatcaggggataacgcaggaaagaacatgtgagcaaaaggccagcaaa |
| | aggccaggaaccgtaaaaaggccgcgttgctggcgtttttccataggctccgccc |
| | ccctgacgagcatcacaaaaatcgacgctcaagtcagaggtggcgaaacccgaca |
| | ggactataaagataccaggcgtttccccctggaagctccctcgtgcgctctcctg |
| | ttccgaccctgccgcttaccggatacctgtccgcctttctcccttcgggaagcgt |
| | ggcgctttctcatagctcacgctgtaggtatctcagttcggtgtaggtcgttcgc |
| | tccaagctgggctgtgtgcacgaaccccccgttcagcccgaccgctgcgccttat |
| | ccggtaactatcgtcttgagtccaacccggtaagacacgacttatcgccactggc |
| | agcagccactggtaacaggattagcagagcgaggtatgtaggcggtgctacagag |
| | ttcttgaagtggtggcctaactacggctacactagaagaacagtatttggtatct |
| | gcgctctgctgaagccagttaccttcggaaaaagagttggtagctcttgatccgg |
| | caaacaaaccaccgctggtagcggtttttttgtttgcaagcagcagattacgcgc |
| | agaaaaaaaggatctcaagaagatcctttgatcttttctacggggtctgacgctc |
| | agtggaacgaatgtggtaatgctctgccagtgttacaaccaattaaccaattctg |
| | attagaaaaactcatcgagcatcaaatgaaactgcaatttattcatatcaggatt |
| | atcaataccatatttttgaaaaagccgtttctgtaatgaaggagaaaactcaccg |
| | aggcagttccataggatggcaagatcctggtatcggtctgcgattccgactcgtc |
| | caacatcaatacaacctattaatttcccctcgtcaaaaataaggttatcaagtga |
| | gaaatcaccatgagtgacgactgaatccggtgagaatggcaaaagtttatgcatt |
| | tctttccagacttgttcaacaggccagccattacgctcgtcatcaaaatcactcg |
| | catcaaccaaaccgttattcattcgtgattgcgcctgagccagtcgaaatacgcg |
| | atcgctgttaaaaggacaattacaaacaggaatcgaatgcaaccggcgcaggaac |
| | actgccagcgcatcaacaatattttcacctgaatcaggatattcttctaatacct |
| | ggaatgctgtttttccggggatcgcagtggtgagtaaccatgcatcatcaggagt |
| | acggataaaatgcttgatggtcggaagaggcataaattccgtcagccagtttagt |
| | ctgaccatctcatctgtaacatcattggcaacgctacctttgccatgtttcagaa |
| | acaactctggcgcatcgggcttcccatacaagcgatagattgtcgcacctgattg |
| | cccgacattatcgcgagcccatttatacccatataaatcagcatccatgttggaa |
| | tttaatcgcggcctcga |
| | (SEQ ID NO: 17) |
| pAJW_E101 | cgtttcccgttgaatatggctcataacaccccttgtattactgtttatgtaagca |
| | gacagttttattgttcatgatgatattattttatcttgtgcaatgtaacatcaga |
| | gattttgagacacggaatcgacgctcaagtcagaggtcctgacgtcgacggatcg |
| | ggagatctcccgatcccctatggtgcactctcagtacaatctgctctgatgccgc |
| | atagttaagccagtatctgctccctgcttgtgtgttggaggtcgctgagtaGGAT |
| | CCtGAAGACatGGAGTCGCtcttttgcaatgctctttgggaaattatattagcct |
| | aattactaatttcctggccctcaaggtgacttgcttgaacttgccacctTCGCCA |
| | CTAGGGGGCAGCAttggtttacacagggtgaacaaacgttcaattctaaactaat |
| | attctttggtgggaaagtgtgttcattttggtttgttttgaagaagtcacttcat |
| | acctttgaactggggttaagctggttaactcccaaaattccaccagttccaatat |
| | cctatggaaaaaccccaaaaccactCAAAggatctgcgatcgctccggtgcccgt |
| | cagtgggcagagcgcacatcgcccacagtccccgagaagttggggggaggggtcg |
| | gcaattgaacgggtgcctagagaaggtggcgcggggtaaactgggaaagtgatgt |
| | cgtgtactggctccgcctttttcccgaggggggggagaaccgtatataagtgcag |
| | tagtcgccgtgaacgttctttttcgcaacgggtttgccgccagaacacagctgaa |
| | gcttcgaggggctcgcatctctccttcacgcgcccgccgccctacctgaggccgc |
| | catccacgccggttgagtcgcgttctgccgcctcccgcctgtggtgcctcctgaa |
| | ctgcgtccgccgtctaggtaagtttaaagctcaggtcgagTccgggcctttgtcc |
| | ggcgctcccttggagcctacctagactcagccggctctccacgctttgcctgacc |
| | ctgcttgctcaactctacgtctttgtttcgttttctgttctgcgccgttacagat |
| | ccaagctgtgaccggcgcctacGAATGCCGCCACCATGgtgagcaagggcgagga |
| | gctgttcaccggggggtgcccatcctggtcgagctggacggcgacgtaaacggcc |
| | acaagttcagcgtgtccggcgagggcgagggcgatgccacctacggcaagctgac |
| | cctgaagttcatctgcaccaccggcaagctgcccgtgccctggcccaccctcgtg |
| | accaccctgacctacggcgtgcagtgcttcagccgctaccccgaccacatgaagc |
| | agcacgacttcttcaagtccgccatgcccgaaggctacgtccaggagcgcaccat |
| | cttcttcaaggacgacggcaactacaagacccgcgccgaggtgaagttcgagggc |
| | gacaccctggtgaaccgcatcgagctgaagggcatcgacttcaaggaggacggca |
| | acatcctggggcacaagctggagtacaactacaacagccacaacgtctatatcat |
| | ggccgacaagcagaagaacggcatcaaggtgaacttcaagatccgccacaacatc |
| | gaggacggcagcgtgcagctcgccgaccactaccagcagaacacccccatcggcg |
| | acggccccgtgctgctgcccgacaaccactacctgagcacccagtccgccctgag |
| | caaagaccccaacgagaagcgcgatcacatggtcctgctggagttcgtgaccgcc |
| | gccgggatcactctcggcatggacgagctgtacaagtaaAACGCCATgggtggca |
| | tccctgtgacccctccccagtgcctctcctggccctggaagttgccactccagtg |
| | cccaccagccttgtcctaataaaattaagttgcatcattttgtctgactaggtgt |
| | ccttctataatattatggggtggaggggggtggtatggagcaaggggcaagttgg |
| | gaagagaacctgtagggcctgcggggtctattgggaaccaagctggagtgcagtg |
| | gcacaatcttggctcactgcaatctccgcctcctgggttcaagcgattctcctgc |
| | ctcagcctcccgagttgttgggattccaggcatgcatgaccaggctcagctaatt |
| | ttttttttttggtagagTcggggtttcaccatattggccaggctggtgtccaact |
| | cctaatctcaggtgatctacccaccttggcctcccaaattgctgggattacaggc |
| | gtgaaccactgctcccttccctgtccttCAGTGGCAatGTCTTCcGGATCCgggt |
| | catagctgtttcctgtgtgaaattgttatccgctcacaattccacacaacatacg |
| | agccggaagcataaagtgtaaagcctggggtgcctaatgagtgagctaactcaca |
| | ttaattgcgttgcgctcactgcccgctttccagtcgggaaacctgtcgtgccagc |
| | tgcattaatgaatcggccaacgcgcggggagaggcggtttgcgtattgggcgctc |
| | ttccgcttcctcgctcactgactcgctgcgctcggtcgttcggctgcggcgagcg |
| | gtatcagctcactcaaaggcggtaatacggttatccacagaatcaggggataacg |
| | caggaaagaacatgtgagcaaaaggccagcaaaaggccaggaaccgtaaaaaggc |
| | cgcgttgctggcgtttttccataggctccgcccccctgacgagcatcacaaaaat |
| | cgacgctcaagtcagaggtggcgaaacccgacaggactataaagataccaggcgt |
| | ttccccctggaagctccctcgtgcgctctcctgttccgaccctgccgcttaccgg |
| | atacctgtccgcctttctcccttcgggaagcgtggcgctttctcatagctcacgc |
| | tgtaggtatctcagttcggtgtaggtcgttcgctccaagctgggctgtgtgcacg |
| | aaccccccgttcagcccgaccgctgcgccttatccggtaactatcgtcttgagtc |
| | caacccggtaagacacgacttatcgccactggcagcagccactggtaacaggatt |
| | agcagagcgaggtatgtaggcggtgctacagagttcttgaagtggtggcctaact |
| | acggctacactagaagaacagtatttggtatctgcgctctgctgaagccagttac |
| | cttcggaaaaagagttggtagctcttgatccggcaaacaaaccaccgctggtagc |
| | ggtttttttgtttgcaagcagcagattacgcgcagaaaaaaaggatctcaagaag |
| | atcctttgatcttttctacggggtctgacgctcagtggaacgaatgtggtaatgc |
| | tctgccagtgttacaaccaattaaccaattctgattagaaaaactcatcgagcat |
| | caaatgaaactgcaatttattcatatcaggattatcaataccatatttttgaaaa |
| | agccgtttctgtaatgaaggagaaaactcaccgaggcagttccataggatggcaa |
| | gatcctggtatcggtctgcgattccgactcgtccaacatcaatacaacctattaa |
| | tttcccctcgtcaaaaataaggttatcaagtgagaaatcaccatgagtgacgact |
| | gaatccggtgagaatggcaaaagtttatgcatttctttccagacttgttcaacag |
| | gccagccattacgctcgtcatcaaaatcactcgcatcaaccaaaccgttattcat |
| | tcgtgattgcgcctgagccagtcgaaatacgcgatcgctgttaaaaggacaatta |
| | caaacaggaatcgaatgcaaccggcgcaggaacactgccagcgcatcaacaatat |
| | tttcacctgaatcaggatattcttctaatacctggaatgctgtttttccggggat |
| | cgcagtggtgagtaaccatgcatcatcaggagtacggataaaatgcttgatggtc |
| | ggaagaggcataaattccgtcagccagtttagtctgaccatctcatctgtaacat |
| | cattggcaacgctacctttgccatgtttcagaaacaactctggcgcatcgggctt |
| | cccatacaagcgatagattgtcgcacctgattgcccgacattatcgcgagcccat |
| | ttatacccatataaatcagcatccatgttggaatttaatcgcggcctcga |
| | (SEQ ID NO: 18) |
| pAJW_E102 | cgtttcccgttgaatatggctcataacaccccttgtattactgtttatgtaagca |
| | gacagttttattgttcatgatgatattattttatcttgtgcaatgtaacatcaga |
| | gattttgagacacggaatcgacgctcaagtcagaggtcctgacgtcgacggatcg |
| | ggagatctcccgatcccctatggtgcactctcagtacaatctgctctgatgccgc |
| | atagttaagccagtatctgctccctgcttgtgtgttggaggtcgctgagtaGGAT |
| | CCtGAAGACatGGAGTCGCtcttttgcaatgctctttgggaaattatattagcct |
| | aattactaatttcctggccctcaaggtgacttgcttgaacttgccacctTCGCCA |
| | CTAGGGGGCAGCAttggtttacacagggtgaacaaacgttcaattctaaactaat |
| | attctttggtgggaaagtgtgttcattttggtttgttttgaagaagtcacttcat |
| | acctttgaactggggttaagctggttaactcccaaaattccaccagttccaatat |
| | cctatggaaaaaccccaaaaccactCAAAGAATTCtaccgggtaggggaggcgct |
| | tttcccaaggcagtctggagcatgcgctttagcagcAccgctgggcacttggcgc |
| | tacacaagtggcctctggcctcgcacacattccacatccaccggtaggcgccaac |
| | cggctccgttctttggtggccccttcgcgccaccttctactcctcccctagtcag |
| | gaagttccccTccgccccgcagctcgcgtcgtgcaggacgtgacaaatggaagta |
| | gcacgtctAactagtctcgtgcagatggacagcaccgctgagcaatggaagcggg |
| | taggcctttggggcagcggccaatagcagctttgctccttcgctttctgggctca |
| | ggggggggggggcccgaaggtcctccggaggcccggcattctgcacgcttcaaaa |
| | gcgcacgtctgccgcgctgttctcctcttcctcatctccgggcctttcgGAATGC |
| | CGCCACCATGgtgagcaagggcgaggagctgttcaccggggtggtgcccatcctg |
| | gtcgagctggacggcgacgtaaacggccacaagttcagcgtgtccggcgagggcg |
| | agggcgatgccacctacggcaagctgaccctgaagttcatctgcaccaccggcaa |
| | gctgcccgtgccctggcccaccctcgtgaccaccctgacctacggcgtgcagtgc |
| | ttcagccgctaccccgaccacatgaagcagcacgacttcttcaagtccgccatgc |
| | ccgaaggctacgtccaggagcgcaccatcttcttcaaggacgacggcaactacaa |
| | gacccgcgccgaggtgaagttcgagggcgacaccctggtgaaccgcatcgagctg |
| | aagggcatcgacttcaaggaggacggcaacatcctggggcacaagctggagtaca |
| | actacaacagccacaacgtctatatcatggccgacaagcagaagaacggcatcaa |
| | ggtgaacttcaagatccgccacaacatcgaggacggcagcgtgcagctcgccgac |
| | cactaccagcagaacacccccatcggcgacggccccgtgctgctgcccgacaacc |
| | actacctgagcacccagtccgccctgagcaaagaccccaacgagaagcgcgatca |
| | catggtcctgctggagttcgtgaccgccgccgggatcactctcggcatggacgag |
| | ctgtacaagtaaAACGCCATgggtggcatccctgtgacccctccccagtgcctct |
| | cctggccctggaagttgccactccagtgcccaccagccttgtcctaataaaatta |
| | agttgcatcattttgtctgactaggtgtccttctataatattatggggtggaggg |
| | gggtggtatggagcaaggggcaagttgggaagagaacctgtagggcctgcggggt |
| | ctattgggaaccaagctggagtgcagtggcacaatcttggctcactgcaatctcc |
| | gcctcctgggttcaagcgattctcctgcctcagcctcccgagttgttgggattcc |
| | aggcatgcatgaccaggctcagctaatttttgtttttttggtagagTcggggttt |
| | caccatattggccaggctggtgtccaactcctaatctcaggtgatctacccacct |
| | tggcctcccaaattgctgggattacaggcgtgaaccactgctcccttccctgtcc |
| | ttCAGTGGCAatGTCTTCcGGATCCgggtcatagctgtttcctgtgtgaaattgt |
| | tatccgctcacaattccacacaacatacgagccggaagcataaagtgtaaagcct |
| | ggggtgcctaatgagtgagctaactcacattaattgcgttgcgctcactgcccgc |
| | tttccagtcgggaaacctgtcgtgccagctgcattaatgaatcggccaacgcgcg |
| | gggagaggcggtttgcgtattgggcgctcttccgcttcctcgctcactgactcgc |
| | tgcgctcggtcgttcggctgcggcgagcggtatcagctcactcaaaggcggtaat |
| | acggttatccacagaatcaggggataacgcaggaaagaacatgtgagcaaaaggc |
| | cagcaaaaggccaggaaccgtaaaaaggccgcgttgctggcgtttttccataggc |
| | tccgcccccctgacgagcatcacaaaaatcgacgctcaagtcagaggtggcgaaa |
| | cccgacaggactataaagataccaggcgtttccccctggaagctccctcgtgcgc |
| | tctcctgttccgaccctgccgcttaccggatacctgtccgcctttctcccttcgg |
| | gaagcgtggcgctttctcatagctcacgctgtaggtatctcagttcggtgtaggt |
| | cgttcgctccaagctgggctgtgtgcacgaaccccccgttcagcccgaccgctgc |
| | gccttatccggtaactatcgtcttgagtccaacccggtaagacacgacttatcgc |
| | cactggcagcagccactggtaacaggattagcagagcgaggtatgtaggcggtgc |
| | tacagagttcttgaagtggtggcctaactacggctacactagaagaacagtattt |
| | ggtatctgcgctctgctgaagccagttaccttcggaaaaagagttggtagctctt |
| | gatccggcaaacaaaccaccgctggtagcggtttttttgtttgcaagcagcagat |
| | tacgcgcagaaaaaaaggatctcaagaagatcctttgatcttttctacggggtct |
| | gacgctcagtggaacgaatgtggtaatgctctgccagtgttacaaccaattaacc |
| | aattctgattagaaaaactcatcgagcatcaaatgaaactgcaatttattcatat |
| | caggattatcaataccatatttttgaaaaagccgtttctgtaatgaaggagaaaa |
| | ctcaccgaggcagttccataggatggcaagatcctggtatcggtctgcgattccg |
| | actcgtccaacatcaatacaacctattaatttcccctcgtcaaaaataaggttat |
| | caagtgagaaatcaccatgagtgacgactgaatccggtgagaatggcaaaagttt |
| | atgcatttctttccagacttgttcaacaggccagccattacgctcgtcatcaaaa |
| | tcactcgcatcaaccaaaccgttattcattcgtgattgcgcctgagccagtcgaa |
| | atacgcgatcgctgttaaaaggacaattacaaacaggaatcgaatgcaaccggcg |
| | caggaacactgccagcgcatcaacaatattttcacctgaatcaggatattcttct |
| | aatacctggaatgctgtttttccggggatcgcagtggtgagtaaccatgcatcat |
| | caggagtacggataaaatgcttgatggtcggaagaggcataaattccgtcagcca |
| | gtttagtctgaccatctcatctgtaacatcattggcaacgctacctttgccatgt |
| | ttcagaaacaactctggcgcatcgggcttcccatacaagcgatagattgtcgcac |
| | ctgattgcccgacattatcgcgagcccatttatacccatataaatcagcatccat |
| | gttggaatttaatcgcggcctcga |
| | (SEQ ID NO: 19) |
| pAJW_P26 | agcgcaaatcttttttaaccctagaaagatagtctgcgtaaaattgacgcatgca |
| | ttcttgaaatattgctctctctttctaaatagcgcgaatccgtcgctgtgcattt |
| | aggacatctcagtcgccgcttggagctcccgtgaggcgtgcttgtcaatgcggta |
| | agtgtcactgattttgaactataacgaccgcgtgagtcaaaatgacgcatgatta |
| | tcttttacgtgacttttaagatttaactcatacgataattatattgttatttcat |
| | gttctacttacgtgataacttattatatatatattttcttgttatagatatcatc |
| | aactttgtatagaaaagttgctcgacattgattattgactagttattaatagtaa |
| | tcaattacggggtcattagttcatagcccatatatggagttccgcgttacataac |
| | ttacggtaaatggcccgcctggctgaccgcccaacgacccccgcccattgacgtc |
| | aataatgacgtatgttcccatagtaacgccaatagggactttccattgacgtcaa |
| | tgggtggagtatttacggtaaactgcccacttggcagtacatcaagtgtatcata |
| | tgccaagtacgccccctattgacgtcaatgacggtaaatggcccgcctggcatta |
| | tgcccagtacatgaccttatgggactttcctacttggcagtacatctacgtatta |
| | gtcatcgctattaccatggtcgaggtgagccccacgttctgcttcactctcccca |
| | tctcccccccctccccacccccaattttgtatttatttattttttaattattttg |
| | tgcagcgatgggggcggggggggggggggggcgcgcgccaggcggggggggcggg |
| | gcgaggggcggggggggcgaggcggagaggtgcggcggcagccaatcagagcggc |
| | gcgctccgaaagtttccttttatggcgaggggggggggggccctataaaaagcga |
| | agcgcgcggggggggagtcgctgcgcgctgccttcgccccgtgccccgctccgcc |
| | gccgcctcgcgccgcccgccccggctctgactgaccgcgttactcccacaggtga |
| | gcgggcgggacggcccttctcctccgggctgtaattagcgcttggtttaatgacg |
| | gcttgtttcttttctgtggctgcgtgaaagccttgaggggctccgggagggccct |
| | ttgtgcggggggagcggctcggggggtgcgtgcgtgtgtgtgtgcgtggggagcg |
| | ccgcgtgcggctccgcgctgcccggcggctgtgagcgctgcgggcgcggcgcggg |
| | gctttgtgcgctccgcagtgtgcgcgaggggagcgcggccgggggcggtgccccg |
| | cggtgcggggggggctgcgaggggaacaaaggctgcgtgcggggtgtgtgcgtgg |
| | gggggtgagcagggggtgtgggcgcgtcggtcgggctgcaaccccccctgcaccc |
| | ccctccccgagttgctgagcacggcccggcttcgggtgcggggctccgtacgggg |
| | cgtggcgcggggctcgccgtgccgggcggggggggcggcaggtgggggtgccggg |
| | cggggcggggccgcctcgggccggggagggctcgggggaggggcgcggcggcccc |
| | cggagcgccggcggctgtcgaggcgcggcgagccgcagccattgccttttatggt |
| | aatcgtgcgagagggcgcagggacttcctttgtcccaaatctgtgcggagccgaa |
| | atctgggaggcgccgccgcaccccctctagcgggcgcggggcgaagcggtgcggc |
| | gccggcaggaaggaaatgggcggggagggccttcgtgcgtcgccgcgccgccgtc |
| | cccttctccctctccagcctcggggctgtccgcggggggacggctgccttcgggg |
| | gggacggggcagggcggggttcggcttctggcgtgtgaccggcggctctagagcc |
| | tctgctaaccatgttcatgccttcttctttttcctacagctcctgggcaacgtgc |
| | tggttattgtgctgtctcatcattttggcaaagaattgcaagtttgtacaaaaaa |
| | gcaggctGAATGCCGCCACCATGGCTaccgagtataaaccgacggtccgacttgc |
| | gactcgggatgacgttccacgcgccgtgaggactctggccgccgccttcgctgac |
| | tacccggccacacggcacacagttgaccccgacagacacatagagcgggtcacag |
| | agttgcaagaacttttcctgacgcgggttggActcgatattggaaaagtttgggt |
| | tgccgacgatggtgccgcagttgcggtgtggactactccagaaagtgtagaagct |
| | ggagccgtattcgctgaaatagggccacggatggcagagctgtctggtagcaggc |
| | tcgcagcgcagcagcagatggaaggCcttctcgctccgcatcggcctaaggaacc |
| | ggcgtggttcctggctacggtgggagtctcacctgaccatcaaggaaagggattg |
| | ggaagtgctgtcgttcttccaggcgtggaagcggccgaacgagcaggcgtcccag |
| | ccttcttggaAacgagcgcacccaggaatcttccattctacgagcgactgggttt |
| | cactgtgacagcagacgtagaagtccctgaagggcctcggacttggtgcatgacc |
| | agaaagcctggggcttgaAACGCCATgatccagacatgataagatacattgatga |
| | gtttggacaaaccacaactagaatgcagtgaGGCGCGCCTGGATACTTCTGCCTG |
| | CAGGGGAGTCGCtcttttgcaatgctctttgggaaattatattagcctaattact |
| | aatttcctggccctcaaggtgacttgcttgaacttgccacctTCGCCACTAGGGG |
| | GCAGCAttggtttacacagggtgaacaaacgttcaattctaaactaatattcttt |
| | ggtgggaaagtgtgttcattttggtttgttttgaagaagtcacttcatacctttg |
| | aactggggttaagctggttaactcccaaaattccaccagttccaatatcctatgg |
| | aaaaaccccaaaaccactCAAAccagatatactcgttgacattgattattgacta |
| | gttattaatagtaatcaattacggggtcattagttcatagcccatatatggagtt |
| | ccgcgttacataacttacggtaaatggcccgcctggctgaccgcccaacgacccc |
| | cgcccattgacgtcaataatgacgtatgttcccatagtaacgccaatagggactt |
| | tccattgacgtcaatgggtggagtatttacggtaaactgcccacttggcagtaca |
| | tcaagtgtatcatatgccaagtacgccccctattgacgtcaatgacggtaaatgg |
| | cccgcctggcattatgcccagtacatgaccttatgggactttcctacttggcagt |
| | acatctacgtattagtcatcgctattaccatggtgatgcggttttggcagtacat |
| | caatgggcgtggatagcggtttgactcacggggatttccaagtctccaccccatt |
| | gacgtcaatgggagtttgttttggcaccaaaatcaacgggactttccaaaatgtc |
| | gtaacaactccgccccattgacgcaaatgggcggtaggcgtgtacggtgggaggt |
| | ctatataagcagagctctctggctaactagagaacccactgcttactggcttatc |
| | gaaattaatacgactcactatagggagaaccaagcGAATGCCGCCACCATGGCTA |
| | TGgtgagcaagggcgaggagctgttcaccggggtggtgcccatcctggtcgagct |
| | ggacggcgacgtaaacggccacaagttcagcgtgtccggcgagggcgagggcgat |
| | gccacctacggcaagctgaccctgaagttcatctgcaccaccggcaagctgcccg |
| | tgccctggcccaccctcgtgaccaccctgacctacggcgtgcagtgcttcagccg |
| | ctaccccgaccacatgaagcagcacgacttcttcaagtccgccatgcccgaaggc |
| | tacgtccaggagcgcaccatcttcttcaaggacgacggcaactacaagacccgcg |
| | ccgaggtgaagttcgagggcgacaccctggtgaaccgcatcgagctgaagggcat |
| | cgacttcaaggaggacggcaacatcctggggcacaagctggagtacaactacaac |
| | agccacaacgtctatatcatggccgacaagcagaagaacggcatcaaggtgaact |
| | tcaagatccgccacaacatcgaggacggcagcgtgcagctcgccgaccactacca |
| | gcagaacacccccatcggcgacggccccgtgctgctgcccgacaaccactacctg |
| | agcacccagtccgccctgagcaaagaccccaacgagaagcgcgatcacatggtcc |
| | tgctggagttcgtgaccgccgccgggatcactctcggcatggacgagctgtacaa |
| | gtaaAACGCCATgaattcactcctcaggtgcaggctgcctatcagaaggtggtgg |
| | ctggtgtggccaatgccctggctcacaaataccactgagatctttttccctctgc |
| | caaaaattatggggacatcatgaagccccttgagcatctgacttctggctaataa |
| | aggaaatttattttcattgcaatagtgtgttggaattttttgtgtctctcactcg |
| | gaaggacatatgggagggcaaatcatttaaaacatcagaatgagtatttggttta |
| | gagtttggcaacatatgcccatatgctggctgccatgaacaaaggttggctataa |
| | agaggtcatcagtatatgaaacagccccctgctgtccattccttattccatagaa |
| | aagccttgacttgaggttagattttttttatattttgttttgtgttatttttttc |
| | tttaacatccctaaaattttccttacatgttttactagccagatttttcctcctc |
| | tcctgactactcccagtcatagctgtccctcttctcttatggagatCAGTGGCAG |
| | CGGCCGCGCCTGGGCTTACttaattaacgagagcataatattgatatgtgccaaa |
| | gttgtttctgactgactaataagtataatttgtttctattatgtataggttaagc |
| | taattacttattttataatacaacatgactgtttttaaagtacaaaataagttta |
| | tttttgtaaaagagagaatgtttaaaagttttgttactttatagaagaaattttg |
| | agtttttgtttttttttaataaataaataaacataaataaattgtttgttgaatt |
| | tattattagtatgtaagtgtaaatataataaaacttaatatctattcaaattaat |
| | aaataaacctcgatatacagaccgataaaacacatgcgtcaattttacgcatgat |
| | tatctttaacgtacgtcacaatatgattatctttctagggttaaataatagtttc |
| | taatttttttattattcagcctgctgtcgtgaataccgagctccaattcgcccta |
| | tagtgagtcgtattacaattcactggccgtcgttttacaacgtcgtgactgggaa |
| | aaccctggcgttacccaacttaatcgccttgcagcacatccccctttcgccagct |
| | ggcgtaatagcgaagaggcccgcaccgatcgcccttcccaacagttgcgcagcct |
| | gaatggcgaatgggacgcgccctgtagcggcgcattaagcgcggcgggtgtggtg |
| | gttacgcgcagcgtgaccgctacacttgccagcgccctagcgcccgctcctttcg |
| | ctttcttcccttcctttctcgccacgttcgccggctttccccgtcaagctctaaa |
| | tcgggggctccctttagggttccgatttagtgctttacggcacctcgaccccaaa |
| | aaacttgattagggtgatggttcacgtagtgggccatcgccctgatagacggttt |
| | ttcgccctttgacgttggagtccacgttctttaatagtggactcttgttccaaac |
| | tggaacaacactcaaccctatctcggtctattcttttgatttataagggattttg |
| | ccgatttcggcctattggttaaaaaatgagctgatttaacaaaaatttaacgcga |
| | attttaacaaaatattaacgcttacaatttaggtggcacttttcggggaaatgtg |
| | cgcggaacccctatttgtttatttttctaaatacattcaaatatgtatccgctca |
| | tgagacaataaccctgataaatgcttcaataatattgaaaaaggaagagtatgag |
| | tattcaacatttccgtgtcgcccttattcccttttttgcggcattttgccttcct |
| | gtttttgctcacccagaaacgctggtgaaagtaaaagatgctgaagatcagttgg |
| | gtgcacgagtgggttacatcgaactggatctcaacagcggtaagatccttgagag |
| | ttttcgccccgaagaacgttttccaatgatgagcacttttaaagttctgctatgt |
| | ggcgcggtattatcccgtattgacgccgggcaagagcaactcggtcgccgcatac |
| | actattctcagaatgacttggttgagtactcaccagtcacagaaaagcatcttac |
| | ggatggcatgacagtaagagaattatgcagtgctgccataaccatgagtgataac |
| | actgcggccaacttacttctgacaacgatcggaggaccgaaggagctaaccgctt |
| | ttttgcacaacatgggggatcatgtaactcgccttgatcgttgggaaccggagct |
| | gaatgaagccataccaaacgacgagcgtgacaccacgatgcctgtagcaatggca |
| | acaacgttgcgcaaactattaactggcgaactacttactctagcttcccggcaac |
| | aattaatagactggatggaggcggataaagttgcaggaccacttctgcgctcggc |
| | ccttccggctggctggtttattgctgataaatctggagccggtgagcgtgggtct |
| | cgcggtatcattgcagcactggggccagatggtaagccctcccgtatcgtagtta |
| | tctacacgacggggagtcaggcaactatggatgaacgaaatagacagatcgctga |
| | gataggtgcctcactgattaagcattggtaactgtcagaccaagtttactcatat |
| | atactttagattgatttaaaacttcatttttaatttaaaaggatctaggtgaaga |
| | tcctttttgataatctcatgaccaaaatcccttaacgtgagttttcgttccactg |
| | agcgtcagaccccgtagaaaagatcaaaggatcttcttgagatcctttttttctg |
| | cgcgtaatctgctgcttgcaaacaaaaaaaccaccgctaccagcggtggtttgtt |
| | tgccggatcaagagctaccaactctttttccgaaggtaactggcttcagcagagc |
| | gcagataccaaatactgttcttctagtgtagccgtagttaggccaccacttcaag |
| | aactctgtagcaccgcctacatacctcgctctgctaatcctgttaccagtggctg |
| | ctgccagtggcgataagtcgtgtcttaccgggttggactcaagacgatagttacc |
| | ggataaggcgcagcggtcgggctgaacggggggttcgtgcacacagcccagcttg |
| | gagcgaacgacctacaccgaactgagatacctacagcgtgagctatgagaaagcg |
| | ccacgcttcccgaagggagaaaggcggacaggtatccggtaagcggcagggtcgg |
| | aacaggagagcgcacgagggagcttccagggggaaacgcctggtatctttatagt |
| | cctgtcgggtttcgccacctctgacttgagcgtcgatttttgtgatgctcgtcag |
| | gggggcggagcctatggaaaaacgccagcaacgcggcctttttacggttcctggc |
| | cttttgctggccttttgctcacatgttctttcctgcgttatcccctgattctgtg |
| | gataaccgtattaccgcctttgagtgagctgataccgctcgccgcagccgaacga |
| | ccgagcgcagcgagtcagtgagcgaggaagcggaagagcgcccaatacgcaaacc |
| | gcctctccccgcgcgttggccgattcattaatgcagctggcacgacaggtttccc |
| | gactggaaagcgggcagtgagcgcaacgcaattaatgtgagttagctcactcatt |
| | aggcaccccaggctttacactttatgcttccggctcgtatgttgtgtggaattgt |
| | gagcggataacaatttcacacaggaaacagctatgaccatgattacgccaagctc |
| | gaaattaaccctcactaaagggaacaaaagctggtacctcgcgcgacttggtttg |
| | ccattctttagcgcgcgtcgcgtcacacagcttggccacaatgtggtttttgtca |
| | aacgaagattctatgacgtgtttaaagtttaggtcgagtaa |
| | (SEQ ID NO: 20) |
| pAJW_P27 | caggaccacttctgcgctcggcccttccggctggctggtttattgctgataaatc |
| | tggagccggtgagcgtgggtctcgcggtatcattgcagcactggggccagatggt |
| | aagccccccgtatcgtagttatctacacgacggggagtcaggcaactatggatga |
| | acgaaatagacagatcgctgagataggtgcctcactgattaagcattggtaactg |
| | tcagaccaagtttactcatatatactttagattgatttaaaacttcatttttaat |
| | ttaaaaggatctaggtgaagatcctttttgataatctcatgaccaaaatccctta |
| | acgtgagttttcgttccactgagcgtcagaccccgtagaaaagatcaaaggatct |
| | tcttgagatcctttttttctgcgcgtaatctgctgcttgcaaacaaaaaaaccac |
| | cgctaccagcggtggtttgtttgccggatcaagagctaccaactctttttccgaa |
| | ggtaactggcttcagcagagcgcagataccaaatactgttcttctagtgtagccg |
| | tagttaggccaccacttcaagaactctgtagcaccgcctacatacctcgctctgc |
| | taatcctgttaccagtggctgctgccagtggcgataagtcgtgtcttaccgggtt |
| | ggactcaagacgatagttaccggataaggcgcagcggtcgggctgaacggggggt |
| | tcgtgcacacagcccagcttggagcgaacgacctacaccgaactgagatacctac |
| | agcgtgagctatgagaaagcgccacgcttcccgaagggagaaaggcggacaggta |
| | tccggtaagcggcagggtcggaacaggagagcgcacgagggagcttccaggggga |
| | aacgcctggtatctttatagtcctgtcgggtttcgccacctctgacttgagcgtc |
| | gatttttgtgatgctcgtcaggggggggagcctatggaaaaacgccagcaacgcg |
| | gcctttttacggttcctggccttttgctggccttttgctcacatgttctttcctg |
| | cgttatcccctgattctgtggataaccgtattaccgcctttgagtgagctgatac |
| | cgctcgccgcagccgaacgaccgagcgcagcgagtcagtgagcgaggaagcggaa |
| | gagcgcccaatacgcaaaccgcctctccccgcgcgttggccgattcattaatgca |
| | gctggcacgacaggtttcccgactggaaagcgggcagtgagcgcaacgcaattaa |
| | tgtgagttagctcactcattaggcaccccaggctttacactttatgcttccggct |
| | cgtatgttgtgtggaattgtgagcggataacaatttcacacaggaaacagctatg |
| | accatgattacgccaagctcgaaattaaccctcactaaagggaacaaaagctggt |
| | acctcgcgcgacttggtttgccattctttagcgcgcgtcgcgtcacacagcttgg |
| | ccacaatgtggtttttgtcaaacgaagattctatgacgtgtttaaagtttaggtc |
| | gagtaaagcgcaaatcttttttaaccctagaaagatagtctgcgtaaaattgacg |
| | catgcattcttgaaatattgctctctctttctaaatagcgcgaatccgtcgctgt |
| | gcatttaggacatctcagtcgccgcttggagctcccgtgaggcgtgcttgtcaat |
| | gcggtaagtgtcactgattttgaactataacgaccgcgtgagtcaaaatgacgca |
| | tgattatcttttacgtgacttttaagatttaactcatacgataattatattgtta |
| | tttcatgttctacttacgtgataacttattatatatatattttcttgttatagat |
| | atcatcaactttgtatagaaaagttgctcgacattgattattgactagttattaa |
| | tagtaatcaattacggggtcattagttcatagcccatatatggagttccgcgtta |
| | cataacttacggtaaatggcccgcctggctgaccgcccaacgacccccgcccatt |
| | gacgtcaataatgacgtatgttcccatagtaacgccaatagggactttccattga |
| | cgtcaatgggtggagtatttacggtaaactgcccacttggcagtacatcaagtgt |
| | atcatatgccaagtacgccccctattgacgtcaatgacggtaaatggcccgcctg |
| | gcattatgcccagtacatgaccttatgggactttcctacttggcagtacatctac |
| | gtattagtcatcgctattaccatggtcgaggtgagccccacgttctgcttcactc |
| | tccccatctcccccccctccccacccccaattttgtatttatttattttttaatt |
| | attttgtgcagcgatgggggcggggggggggggggggcgcgcgccaggcggggcg |
| | gggggggcgaggggggggcggggcgaggcggagaggtgcggcggcagccaatcag |
| | agcggcgcgctccgaaagtttccttttatggcgaggcggcggcggcggcggccct |
| | ataaaaagcgaagcgcgcggggggggagtcgctgcgcgctgccttcgccccgtgc |
| | cccgctccgccgccgcctcgcgccgcccgccccggctctgactgaccgcgttact |
| | cccacaggtgagcggggggacggcccttctcctccgggctgtaattagcgcttgg |
| | tttaatgacggcttgtttcttttctgtggctgcgtgaaagccttgaggggctccg |
| | ggagggccctttgtgcggggggagcggctcggggggtgcgtgcgtgtgtgtgtgc |
| | gtggggagcgccgcgtgcggctccgcgctgcccggcggctgtgagcgctgcgggc |
| | gcggcgcggggctttgtgcgctccgcagtgtgcgcgaggggagcgcggccggggg |
| | cggtgccccgcggtgcggggggggctgcgaggggaacaaaggctgcgtgcggggt |
| | gtgtgcgtgggggggtgagcagggggtgtgggcgcgtcggtcgggctgcaacccc |
| | ccctgcacccccctccccgagttgctgagcacggcccggcttcgggtgcggggct |
| | ccgtacggggcgtggcgcggggctcgccgtgccgggcggggggggcggcaggtgg |
| | gggtgccgggcggggcggggccgcctcgggccggggagggctcgggggaggggcg |
| | cggcggcccccggagcgccggcggctgtcgaggcgcggcgagccgcagccattgc |
| | cttttatggtaatcgtgcgagagggcgcagggacttcctttgtcccaaatctgtg |
| | cggagccgaaatctgggaggcgccgccgcaccccctctagcgggcgcggggcgaa |
| | gcggtgcggcgccggcaggaaggaaatgggggggagggccttcgtgcgtcgccgc |
| | gccgccgtccccttctccctctccagcctcggggctgtccgcggggggacggctg |
| | ccttcgggggggacggggcagggcggggttcggcttctggcgtgtgaccggcggc |
| | tctagagcctctgctaaccatgttcatgccttcttctttttcctacagctcctgg |
| | gcaacgtgctggttattgtgctgtctcatcattttggcaaagaattgcaagtttg |
| | tacaaaaaagcaggctGAATGCCGCCACCATGGCTaccgagtataaaccgacggt |
| | ccgacttgcgactcgggatgacgttccacgcgccgtgaggactctggccgccgcc |
| | ttcgctgactacccggccacacggcacacagttgaccccgacagacacatagagc |
| | gggtcacagagttgcaagaacttttcctgacgcgggttggActcgatattggaaa |
| | agtttgggttgccgacgatggtgccgcagttgcggtgtggactactccagaaagt |
| | gtagaagctggagccgtattcgctgaaatagggccacggatggcagagctgtctg |
| | gtagcaggctcgcagcgcagcagcagatggaaggCcttctcgctccgcatcggcc |
| | taaggaaccggcgtggttcctggctacggtgggagtctcacctgaccatcaagga |
| | aagggattgggaagtgctgtcgttcttccaggcgtggaagcggccgaacgagcag |
| | gcgtcccagccttcttggaAacgagcgcacccaggaatcttccattctacgagcg |
| | actgggtttcactgtgacagcagacgtagaagtccctgaagggcctcggacttgg |
| | tgcatgaccagaaagcctggggcttgaAACGCCATgatccagacatgataagata |
| | cattgatgagtttggacaaaccacaactagaatgcagtgaGGCGCGCCTGGATAC |
| | TTCTGCCTGCAGGGGAGTCGCtcttttgcaatgctctttgggaaattatattagc |
| | ctaattactaatttcctggccctcaaggtgacttgcttgaacttgccacctTCGC |
| | CACTAGGGGGCAGCAttggtttacacagggtgaacaaacgttcaattctaaacta |
| | atattctttggtgggaaagtgtgttcattttggtttgttttgaagaagtcacttc |
| | atacctttgaactggggttaagctggttaactcccaaaattccaccagttccaat |
| | atcctatggaaaaaccccaaaaccactCAAAccagatatactcgttgacattgat |
| | tattgactagttattaatagtaatcaattacggggtcattagttcatagcccata |
| | tatggagttccgcgttacataacttacggtaaatggcccgcctggctgaccgccc |
| | aacgacccccgcccattgacgtcaataatgacgtatgttcccatagtaacgccaa |
| | tagggactttccattgacgtcaatgggtggagtatttacggtaaactgcccactt |
| | ggcagtacatcaagtgtatcatatgccaagtacgccccctattgacgtcaatgac |
| | ggtaaatggcccgcctggcattatgcccagtacatgaccttatgggactttccta |
| | cttggcagtacatctacgtattagtcatcgctattaccatggtgatgcggttttg |
| | gcagtacatcaatgggcgtggatagcggtttgactcacggggatttccaagtctc |
| | caccccattgacgtcaatgggagtttgttttggcaccaaaatcaacgggactttc |
| | caaaatgtcgtaacaactccgccccattgacgcaaatgggggtaggcgtgtacgg |
| | tgggaggtctatataagcagagctctctggctaactagagaacccactgcttact |
| | ggcttatcgaaattaatacgactcactatagggagaaccaagcGAATGCCGCCAC |
| | CATGGCTGTCAGTAAAGGGGAAgagcttataaaggaaaatatgcacatgaagctc |
| | tacatggagggcactgtagataaccaccatttcaaatgtacctctgaaggggagg |
| | gcaagccatacgaaggtactcaaaccatgcgaataaaagtagttgaaggcgggcc |
| | tcttccctttgcattcgacattctcgcaacctcttttctgtacggcagtaagact |
| | ttcataaaccacactcaaggcattccagacttcttcaagcaatcattccccgaag |
| | gattcacctgggagcgagttactacttatgaggacggaggagtccttactgcaac |
| | ccaagacacctcactgcaagatgggtgcctgatttacaatgtaaagatcagaggg |
| | gtgaatttcacaagcaatgggccagttatgcaaaaaaagacccttggatgggagg |
| | ccttcaccgagacactgtacccagccgatggtggactggagggcaggaatgacat |
| | ggccctcaagctcgtcggaggcagtcacttgattgccaacgccaaaaccacttac |
| | cgctctaagaaacctgctaaaaacctgaagatgcccggcgtctattatgtggact |
| | atcgacttgagagaattaaggaggcaaacaacgagacttatgtcgaacagcatga |
| | agttgccgtggctaggtactgtgatttgcccagcaaattgggtcataaacttaac |
| | TGAAACGCCATgaattcactcctcaggtgcaggctgcctatcagaaggtggtggc |
| | tggtgtggccaatgccctggctcacaaataccactgagatctttttccctctgcc |
| | aaaaattatggggacatcatgaagccccttgagcatctgacttctggctaataaa |
| | ggaaatttattttcattgcaatagtgtgttggaattttttgtgtctctcactcgg |
| | aaggacatatgggagggcaaatcatttaaaacatcagaatgagtatttggtttag |
| | agtttggcaacatatgcccatatgctggctgccatgaacaaaggttggctataaa |
| | gaggtcatcagtatatgaaacagccccctgctgtccattccttattccatagaaa |
| | agccttgacttgaggttagattttttttatattttgttttgtgttatttttttct |
| | ttaacatccctaaaattttccttacatgttttactagccagatttttcctcctct |
| | cctgactactcccagtcatagctgtccctcttctcttatggagatcCAGTGGCAG |
| | CGGCCGCGCCTGGGCTTACttaattaacgagagcataatattgatatgtgccaaa |
| | gttgtttctgactgactaataagtataatttgtttctattatgtataggttaagc |
| | taattacttattttataatacaacatgactgtttttaaagtacaaaataagttta |
| | tttttgtaaaagagagaatgtttaaaagttttgttactttatagaagaaattttg |
| | agtttttgtttttttttaataaataaataaacataaataaattgtttgttgaatt |
| | tattattagtatgtaagtgtaaatataataaaacttaatatctattcaaattaat |
| | aaataaacctcgatatacagaccgataaaacacatgcgtcaattttacgcatgat |
| | tatctttaacgtacgtcacaatatgattatctttctagggttaaataatagtttc |
| | taatttttttattattcagcctgctgtcgtgaataccgagctccaattcgcccta |
| | tagtgagtcgtattacaattcactggccgtcgttttacaacgtcgtgactgggaa |
| | aaccctggcgttacccaacttaatcgccttgcagcacatccccctttcgccagct |
| | ggcgtaatagcgaagaggcccgcaccgatcgcccttcccaacagttgcgcagcct |
| | gaatggcgaatgggacgcgccctgtagcggcgcattaagcgcggcgggtgtggtg |
| | gttacgcgcagcgtgaccgctacacttgccagcgccctagcgcccgctcctttcg |
| | ctttcttcccttcctttctcgccacgttcgccggctttccccgtcaagctctaaa |
| | tcgggggctccctttagggttccgatttagtgctttacggcacctcgaccccaaa |
| | aaacttgattagggtgatggttcacgtagtgggccatcgccctgatagacggttt |
| | ttcgccctttgacgttggagtccacgttctttaatagtggactcttgttccaaac |
| | tggaacaacactcaaccctatctcggtctattcttttgatttataagggattttg |
| | ccgatttcggcctattggttaaaaaatgagctgatttaacaaaaatttaacgcga |
| | attttaacaaaatattaacgcttacaatttaggtggcacttttcggggaaatgtg |
| | cgcggaacccctatttgtttatttttctaaatacattcaaatatgtatccgctca |
| | tgagacaataaccctgataaatgcttcaataatattgaaaaaggaagagtatgag |
| | tattcaacatttccgtgtcgcccttattcccttttttgcggcattttgccttcct |
| | gtttttgctcacccagaaacgctggtgaaagtaaaagatgctgaagatcagttgg |
| | gtgcacgagtgggttacatcgaactggatctcaacagcggtaagatccttgagag |
| | ttttcgccccgaagaacgttttccaatgatgagcacttttaaagttctgctatgt |
| | ggcgcggtattatcccgtattgacgccgggcaagagcaactcggtcgccgcatac |
| | actattctcagaatgacttggttgagtactcaccagtcacagaaaagcatcttac |
| | ggatggcatgacagtaagagaattatgcagtgctgccataaccatgagtgataac |
| | actgcggccaacttacttctgacaacgatcggaggaccgaaggagctaaccgctt |
| | ttttgcacaacatgggggatcatgtaactcgccttgatcgttgggaaccggagct |
| | gaatgaagccataccaaacgacgagcgtgacaccacgatgcctgtagcaatggca |
| | acaacgttgcgcaaactattaactggcgaactacttactctagcttcccggcaac |
| | aattaatagactggatggaggcggataaagttg |
| | (SEQ ID NO: 21) |
| pAJW_P28 | ctctgccaaaaattatggggacatcatgaagccccttgagcatctgacttctggc |
| | taataaaggaaatttattttcattgcaatagtgtgttggaattttttgtgtctct |
| | cactcggaaggacatatgggagggcaaatcatttaaaacatcagaatgagtattt |
| | ggtttagagtttggcaacatatgcccatatgctggctgccatgaacaaaggttgg |
| | ctataaagaggtcatcagtatatgaaacagccccctgctgtccattccttattcc |
| | atagaaaagccttgacttgaggttagattttttttatattttgttttgtgttatt |
| | tttttctttaacatccctaaaattttccttacatgttttactagccagatttttc |
| | ctcctctcctgactactcccagtcatagctgtccctcttctcttatggagatCAG |
| | TGGCAGCGGCCGCGCCTGGGCTTACttaattaacgagagcataatattgatatgt |
| | gccaaagttgtttctgactgactaataagtataatttgtttctattatgtatagg |
| | ttaagctaattacttattttataatacaacatgactgtttttaaagtacaaaata |
| | agtttatttttgtaaaagagagaatgtttaaaagttttgttactttatagaagaa |
| | attttgagtttttgtttttttttaataaataaataaacataaataaattgtttgt |
| | tgaatttattattagtatgtaagtgtaaatataataaaacttaatatctattcaa |
| | attaataaataaacctcgatatacagaccgataaaacacatgcgtcaattttacg |
| | catgattatctttaacgtacgtcacaatatgattatctttctagggttaaataat |
| | agtttctaatttttttattattcagcctgctgtcgtgaataccgagctccaattc |
| | gccctatagtgagtcgtattacaattcactggccgtcgttttacaacgtcgtgac |
| | tgggaaaaccctggcgttacccaacttaatcgccttgcagcacatccccctttcg |
| | ccagctggcgtaatagcgaagaggcccgcaccgatcgcccttcccaacagttgcg |
| | cagcctgaatggcgaatgggacgcgccctgtagcggcgcattaagcgcggcgggt |
| | gtggtggttacgcgcagcgtgaccgctacacttgccagcgccctagcgcccgctc |
| | ctttcgctttcttcccttcctttctcgccacgttcgccggctttccccgtcaagc |
| | tctaaatcgggggctccctttagggttccgatttagtgctttacggcacctcgac |
| | cccaaaaaacttgattagggtgatggttcacgtagtgggccatcgccctgataga |
| | cggtttttcgccctttgacgttggagtccacgttctttaatagtggactcttgtt |
| | ccaaactggaacaacactcaaccctatctcggtctattcttttgatttataaggg |
| | attttgccgatttcggcctattggttaaaaaatgagctgatttaacaaaaattta |
| | acgcgaattttaacaaaatattaacgcttacaatttaggtggcacttttcgggga |
| | aatgtgcgcggaacccctatttgtttatttttctaaatacattcaaatatgtatc |
| | cgctcatgagacaataaccctgataaatgcttcaataatattgaaaaaggaagag |
| | tatgagtattcaacatttccgtgtcgcccttattcccttttttgcggcattttgc |
| | cttcctgtttttgctcacccagaaacgctggtgaaagtaaaagatgctgaagatc |
| | agttgggtgcacgagtgggttacatcgaactggatctcaacagcggtaagatcct |
| | tgagagttttcgccccgaagaacgttttccaatgatgagcacttttaaagttctg |
| | ctatgtggcgcggtattatcccgtattgacgccgggcaagagcaactcggtcgcc |
| | gcatacactattctcagaatgacttggttgagtactcaccagtcacagaaaagca |
| | tcttacggatggcatgacagtaagagaattatgcagtgctgccataaccatgagt |
| | gataacactgcggccaacttacttctgacaacgatcggaggaccgaaggagctaa |
| | ccgcttttttgcacaacatgggggatcatgtaactcgccttgatcgttgggaacc |
| | ggagctgaatgaagccataccaaacgacgagcgtgacaccacgatgcctgtagca |
| | atggcaacaacgttgcgcaaactattaactggcgaactacttactctagcttccc |
| | ggcaacaattaatagactggatggaggcggataaagttgcaggaccacttctgcg |
| | ctcggcccttccggctggctggtttattgctgataaatctggagccggtgagcgt |
| | gggtctcgcggtatcattgcagcactggggccagatggtaagccctcccgtatcg |
| | tagttatctacacgacggggagtcaggcaactatggatgaacgaaatagacagat |
| | cgctgagataggtgcctcactgattaagcattggtaactgtcagaccaagtttac |
| | tcatatatactttagattgatttaaaacttcatttttaatttaaaaggatctagg |
| | tgaagatcctttttgataatctcatgaccaaaatcccttaacgtgagttttcgtt |
| | ccactgagcgtcagaccccgtagaaaagatcaaaggatcttcttgagatcctttt |
| | tttctgcgcgtaatctgctgcttgcaaacaaaaaaaccaccgctaccagcggtgg |
| | tttgtttgccggatcaagagctaccaactctttttccgaaggtaactggcttcag |
| | cagagcgcagataccaaatactgttcttctagtgtagccgtagttaggccaccac |
| | ttcaagaactctgtagcaccgcctacatacctcgctctgctaatcctgttaccag |
| | tggctgctgccagtggcgataagtcgtgtcttaccgggttggactcaagacgata |
| | gttaccggataaggcgcagcggtcgggctgaacggggggttcgtgcacacagccc |
| | agcttggagcgaacgacctacaccgaactgagatacctacagcgtgagctatgag |
| | aaagcgccacgcttcccgaagggagaaaggcggacaggtatccggtaagcggcag |
| | ggtcggaacaggagagcgcacgagggagcttccagggggaaacgcctggtatctt |
| | tatagtcctgtcgggtttcgccacctctgacttgagcgtcgatttttgtgatgct |
| | cgtcaggggggcggagcctatggaaaaacgccagcaacgcggcctttttacggtt |
| | cctggccttttgctggccttttgctcacatgttctttcctgcgttatcccctgat |
| | tctgtggataaccgtattaccgcctttgagtgagctgataccgctcgccgcagcc |
| | gaacgaccgagcgcagcgagtcagtgagcgaggaagcggaagagcgcccaatacg |
| | caaaccgcctctccccgcgcgttggccgattcattaatgcagctggcacgacagg |
| | tttcccgactggaaagcgggcagtgagcgcaacgcaattaatgtgagttagctca |
| | ctcattaggcaccccaggctttacactttatgcttccggctcgtatgttgtgtgg |
| | aattgtgagcggataacaatttcacacaggaaacagctatgaccatgattacgcc |
| | aagctcgaaattaaccctcactaaagggaacaaaagctggtacctcgcgcgactt |
| | ggtttgccattctttagcgcgcgtcgcgtcacacagcttggccacaatgtggttt |
| | ttgtcaaacgaagattctatgacgtgtttaaagtttaggtcgagtaaagcgcaaa |
| | tcttttttaaccctagaaagatagtctgcgtaaaattgacgcatgcattcttgaa |
| | atattgctctctctttctaaatagcgcgaatccgtcgctgtgcatttaggacatc |
| | tcagtcgccgcttggagctcccgtgaggcgtgcttgtcaatgcggtaagtgtcac |
| | tgattttgaactataacgaccgcgtgagtcaaaatgacgcatgattatcttttac |
| | gtgacttttaagatttaactcatacgataattatattgttatttcatgttctact |
| | tacgtgataacttattatatatatattttcttgttatagatatcatcaactttgt |
| | atagaaaagttgctcgacattgattattgactagttattaatagtaatcaattac |
| | ggggtcattagttcatagcccatatatggagttccgcgttacataacttacggta |
| | aatggcccgcctggctgaccgcccaacgacccccgcccattgacgtcaataatga |
| | cgtatgttcccatagtaacgccaatagggactttccattgacgtcaatgggtgga |
| | gtatttacggtaaactgcccacttggcagtacatcaagtgtatcatatgccaagt |
| | acgccccctattgacgtcaatgacggtaaatggcccgcctggcattatgcccagt |
| | acatgaccttatgggactttcctacttggcagtacatctacgtattagtcatcgc |
| | tattaccatggtcgaggtgagccccacgttctgcttcactctccccatctccccc |
| | ccctccccacccccaattttgtatttatttattttttaattattttgtgcagcga |
| | tgggggcggggggggggggggggcgcgcgccaggcggggcggggcggggcgaggg |
| | gcggggcggggcgaggcggagaggtgcggcggcagccaatcagagcggcgcgctc |
| | cgaaagtttccttttatggcgaggcggcggcggcggcggccctataaaaagcgaa |
| | gcgcgcggcgggcgggagtcgctgcgcgctgccttcgccccgtgccccgctccgc |
| | cgccgcctcgcgccgcccgccccggctctgactgaccgcgttactcccacaggtg |
| | agcggggggacggcccttctcctccgggctgtaattagcgcttggtttaatgacg |
| | gcttgtttcttttctgtggctgcgtgaaagccttgaggggctccgggagggccct |
| | ttgtgcggggggagcggctcggggggtgcgtgcgtgtgtgtgtgcgtggggagcg |
| | ccgcgtgcggctccgcgctgcccggcggctgtgagcgctgcgggcgcggcgcggg |
| | gctttgtgcgctccgcagtgtgcgcgaggggagcgcggccgggggcggtgccccg |
| | cggtgcggggggggctgcgaggggaacaaaggctgcgtgcggggtgtgtgcgtgg |
| | gggggtgagcagggggtgtgggcgcgtcggtcgggctgcaaccccccctgcaccc |
| | ccctccccgagttgctgagcacggcccggcttcgggtgcggggctccgtacgggg |
| | cgtggcgcggggctcgccgtgccgggcggggggtggcggcaggtgggggtgccgg |
| | gcggggcggggccgcctcgggccggggagggctcgggggaggggcgcggcggccc |
| | ccggagcgccggcggctgtcgaggcgcggcgagccgcagccattgccttttatgg |
| | taatcgtgcgagagggcgcagggacttcctttgtcccaaatctgtgcggagccga |
| | aatctgggaggcgccgccgcaccccctctagcgggcgcggggcgaagcggtgcgg |
| | cgccggcaggaaggaaatgggcggggagggccttcgtgcgtcgccgcgccgccgt |
| | ccccttctccctctccagcctcggggctgtccgcggggggacggctgccttcggg |
| | ggggacggggcagggcggggttcggcttctggcgtgtgaccggcggctctagagc |
| | ctctgctaaccatgttcatgccttcttctttttcctacagctcctgggcaacgtg |
| | ctggttattgtgctgtctcatcattttggcaaagaattgcaagtttgtacaaaaa |
| | agcaggctGAATGCCGCCACCATGGCTaccgagtataaaccgacggtccgacttg |
| | cgactcgggatgacgttccacgcgccgtgaggactctggccgccgccttcgctga |
| | ctacccggccacacggcacacagttgaccccgacagacacatagagcgggtcaca |
| | gagttgcaagaacttttcctgacgcgggttggActcgatattggaaaagtttggg |
| | ttgccgacgatggtgccgcagttgcggtgtggactactccagaaagtgtagaagc |
| | tggagccgtattcgctgaaatagggccacggatggcagagctgtctggtagcagg |
| | ctcgcagcgcagcagcagatggaaggCcttctcgctccgcatcggcctaaggaac |
| | cggcgtggttcctggctacggtgggagtctcacctgaccatcaaggaaagggatt |
| | gggaagtgctgtcgttcttccaggcgtggaagcggccgaacgagcaggcgtccca |
| | gccttcttggaAacgagcgcacccaggaatcttccattctacgagcgactgggtt |
| | tcactgtgacagcagacgtagaagtccctgaagggcctcggacttggtgcatgac |
| | cagaaagcctggggcttgaAACGCCATgatccagacatgataagatacattgatg |
| | agtttggacaaaccacaactagaatgcagtgaGGCGCGCCTGGATACTTCTGCCT |
| | GCAGGGGAGTCGCtcttttgcaatgctctttgggaaattatattagcctaattac |
| | taatttcctggccctcaaggtgacttgcttgaacttgccacctTCGCCACTAGGG |
| | GGCAGCAttggtttacacagggtgaacaaacgttcaattctaaactaatattctt |
| | tggtgggaaagtgtgttcattttggtttgttttgaagaagtcacttcataccttt |
| | gaactggggttaagctggttaactcccaaaattccaccagttccaatatcctatg |
| | gaaaaaccccaaaaccactCAAAccagatatactcgttgacattgattattgact |
| | agttattaatagtaatcaattacggggtcattagttcatagcccatatatggagt |
| | tccgcgttacataacttacggtaaatggcccgcctggctgaccgcccaacgaccc |
| | ccgcccattgacgtcaataatgacgtatgttcccatagtaacgccaatagggact |
| | ttccattgacgtcaatgggtggagtatttacggtaaactgcccacttggcagtac |
| | atcaagtgtatcatatgccaagtacgccccctattgacgtcaatgacggtaaatg |
| | gcccgcctggcattatgcccagtacatgaccttatgggactttcctacttggcag |
| | tacatctacgtattagtcatcgctattaccatggtgatgcggttttggcagtaca |
| | tcaatgggcgtggatagcggtttgactcacggggatttccaagtctccaccccat |
| | tgacgtcaatgggagtttgttttggcaccaaaatcaacgggactttccaaaatgt |
| | gtaacaactccgccccattgacgcaaatgggcggtaggcgtgtacggtgggaggt |
| | ctatataagcagagctctctggctaactagagaacccactgcttactggcttatc |
| | gaaattaatacgactcactatagggagaaccaagcGAATGCCGCCACCATGGCTg |
| | ctgaaggTtccgtcgccaggcagcctgacctcttgacctgcgacgatgagccgat |
| | ccatatccccggtgccatccaaccgcatggactgctgctcgccctcgccgccgac |
| | atgacgatcgttgccggcagcgacaaccttcccgaactcaccggactggcgatcg |
| | gcgccctgatcggccgctctgcggccgatgtGttcgactcggaAacgcacaaccg |
| | tctgacgatcgccttggccgagcccggggcggccgtcggagcaccgatcactgtc |
| | ggcttcacgatgcgaaaggacgcaggcttcatcggctcctggcatcgccatgatc |
| | agctcatcttcctGgagctGgagcctccccagcgggacgtcgccgagccgcaggc |
| | gttcttccgccgcaccaacagcgccatccgccgcctgcaggccgccgaaaccttg |
| | gaaagcgcctgcgccgccgcggcgcaagaggtgcggaagattaccggcttcgatc |
| | gggtgatgatctatcgcttcgcctccgacttcagcggcgaagtgatcgcagagga |
| | tcggtgcgccgaggtcgagtcaaaactaggcctgcactatcctgcctcaaccgtg |
| | ccggcgcaggcccgtcggctctataccatcaacccggtacggatcattcccgata |
| | tcaattatcggccggtgccggtcaccccagacctcaatccggtcaccgggcggcc |
| | gattgatcttagcttcgccatcctgcgcagcgtGtcgcccgtccatctggaGttc |
| | atgcgcaacataggcatgcacggcacgatgtcgatctcgattttgcgcggcgagc |
| | gactgtggggattgatcgtttgccatcaccgaacgccgtactacgtcgatctcga |
| | tggccgccaagcctgcgagctagtcgcccaggttctggcctggcagatcggcgtg |
| | atggaagagTAAAACGCCATgaattcactcctcaggtgcaggctgcctatcagaa |
| | ggtggtggctggtgtggccaatgccctggctcacaaataccactgagatcttttt |
| | cc |
| | (SEQ ID NO: 22) |
| pAJW_P29 | caggaccacttctgcgctcggcccttccggctggctggtttattgctgataaatc |
| | tggagccggtgagcgtgggtctcgcggtatcattgcagcactggggccagatggt |
| | aagccctcccgtatcgtagttatctacacgacggggagtcaggcaactatggatg |
| | aacgaaatagacagatcgctgagataggtgcctcactgattaagcattggtaact |
| | gtcagaccaagtttactcatatatactttagattgatttaaaacttcatttttaa |
| | tttaaaaggatctaggtgaagatcctttttgataatctcatgaccaaaatccctt |
| | aacgtgagttttcgttccactgagcgtcagaccccgtagaaaagatcaaaggatc |
| | ttcttgagatcctttttttctgcgcgtaatctgctgcttgcaaacaaaaaaacca |
| | ccgctaccagcggtggtttgtttgccggatcaagagctaccaactctttttccga |
| | aggtaactggcttcagcagagcgcagataccaaatactgttcttctagtgtagcc |
| | gtagttaggccaccacttcaagaactctgtagcaccgcctacatacctcgctctg |
| | ctaatcctgttaccagtggctgctgccagtggcgataagtcgtgtcttaccgggt |
| | tggactcaagacgatagttaccggataaggcgcagcggtcgggctgaacgggggg |
| | ttcgtgcacacagcccagcttggagcgaacgacctacaccgaactgagataccta |
| | cagcgtgagctatgagaaagcgccacgcttcccgaagggagaaaggcggacaggt |
| | atccggtaagcggcagggtcggaacaggagagcgcacgagggagcttccaggggg |
| | aaacgcctggtatctttatagtcctgtcgggtttcgccacctctgacttgagcgt |
| | cgatttttgtgatgctcgtcaggggggcggagcctatggaaaaacgccagcaacg |
| | cggcctttttacggttcctggccttttgctggccttttgctcacatgttctttcc |
| | tgcgttatcccctgattctgtggataaccgtattaccgcctttgagtgagctgat |
| | accgctcgccgcagccgaacgaccgagcgcagcgagtcagtgagcgaggaagcgg |
| | aagagcgcccaatacgcaaaccgcctctccccgcgcgttggccgattcattaatg |
| | cagctggcacgacaggtttcccgactggaaagcgggcagtgagcgcaacgcaatt |
| | aatgtgagttagctcactcattaggcaccccaggctttacactttatgcttccgg |
| | ctcgtatgttgtgtggaattgtgagcggataacaatttcacacaggaaacagcta |
| | tgaccatgattacgccaagctcgaaattaaccctcactaaagggaacaaaagctg |
| | gtacctcgcgcgacttggtttgccattctttagcgcgcgtcgcgtcacacagctt |
| | ggccacaatgtggtttttgtcaaacgaagattctatgacgtgtttaaagtttagg |
| | tcgagtaaagcgcaaatcttttttaaccctagaaagatagtctgcgtaaaattga |
| | cgcatgcattcttgaaatattgctctctctttctaaatagcgcgaatccgtcgct |
| | gtgcatttaggacatctcagtcgccgcttggagctcccgtgaggcgtgcttgtca |
| | atgcggtaagtgtcactgattttgaactataacgaccgcgtgagtcaaaatgacg |
| | catgattatcttttacgtgacttttaagatttaactcatacgataattatattgt |
| | tatttcatgttctacttacgtgataacttattatatatatattttcttgttatag |
| | atatcatcaactttgtatagaaaagttgctcgacattgattattgactagttatt |
| | aatagtaatcaattacggggtcattagttcatagcccatatatggagttccgcgt |
| | tacataacttacggtaaatggcccgcctggctgaccgcccaacgacccccgccca |
| | ttgacgtcaataatgacgtatgttcccatagtaacgccaatagggactttccatt |
| | gacgtcaatgggtggagtatttacggtaaactgcccacttggcagtacatcaagt |
| | gtatcatatgccaagtacgccccctattgacgtcaatgacggtaaatggcccgcc |
| | tggcattatgcccagtacatgaccttatgggactttcctacttggcagtacatct |
| | acgtattagtcatcgctattaccatggtcgaggtgagccccacgttctgcttcac |
| | tctccccatctcccccccctccccacccccaattttgtatttatttattttttaa |
| | ttattttgtgcagcgatgggggcggggggggggggggggcgcgcgccaggcgggg |
| | cggggcggggcgaggggggggcggggcgaggcggagaggtgcggcggcagccaat |
| | cagagcggcgcgctccgaaagtttccttttatggcgaggcggcggcggcggcggc |
| | cctataaaaagcgaagcgcgcggggggggagtcgctgcgcgctgccttcgccccg |
| | tgccccgctccgccgccgcctcgcgccgcccgccccggctctgactgaccgcgtt |
| | actcccacaggtgagcggggggacggcccttctcctccgggctgtaattagcgct |
| | tggtttaatgacggcttgtttcttttctgtggctgcgtgaaagccttgaggggct |
| | ccgggagggccctttgtgcggggggagcggctcggggggtgcgtgcgtgtgtgtg |
| | tgcgtggggagcgccgcgtgcggctccgcgctgcccggcggctgtgagcgctgcg |
| | ggcgcggcgcggggctttgtgcgctccgcagtgtgcgcgaggggagcgcggccgg |
| | gggcggtgccccgcggtgcggggggggctgcgaggggaacaaaggctgcgtgcgg |
| | ggtgtgtgcgtgggggggtgagcagggggtgtgggcgcgtcggtcgggctgcaac |
| | cccccctgcacccccctccccgagttgctgagcacggcccggcttcgggtgcggg |
| | gctccgtacggggcgtggcgcggggctcgccgtgccgggcggggggggcggcagg |
| | tgggggtgccgggcggggcggggccgcctcgggccggggagggctcgggggaggg |
| | gcgcggcggcccccggagcgccggcggctgtcgaggcgcggcgagccgcagccat |
| | tgccttttatggtaatcgtgcgagagggcgcagggacttcctttgtcccaaatct |
| | gtgcggagccgaaatctgggaggcgccgccgcaccccctctagcgggcgcggggc |
| | gaagcggtgcggcgccggcaggaaggaaatgggcggggagggccttcgtgcgtcg |
| | ccgcgccgccgtccccttctccctctccagcctcggggctgtccgcggggggacg |
| | gctgccttcgggggggacggggcagggcggggttcggcttctggcgtgtgaccgg |
| | cggctctagagcctctgctaaccatgttcatgccttcttctttttcctacagctc |
| | ctgggcaacgtgctggttattgtgctgtctcatcattttggcaaagaattgcaag |
| | tttgtacaaaaaagcaggctGAATGCCGCCACCATGGCTaccgagtataaaccga |
| | cggtccgacttgcgactcgggatgacgttccacgcgccgtgaggactctggccgc |
| | cgccttcgctgactacccggccacacggcacacagttgaccccgacagacacata |
| | gagcgggtcacagagttgcaagaacttttcctgacgcgggttggActcgatattg |
| | gaaaagtttgggttgccgacgatggtgccgcagttgcggtgtggactactccaga |
| | aagtgtagaagctggagccgtattcgctgaaatagggccacggatggcagagctg |
| | tctggtagcaggctcgcagcgcagcagcagatggaaggCcttctcgctccgcatc |
| | ggcctaaggaaccggcgtggttcctggctacggtgggagtctcacctgaccatca |
| | aggaaagggattgggaagtgctgtcgttcttccaggcgtggaagcggccgaacga |
| | gcaggcgtcccagccttcttggaAacgagcgcacccaggaatcttccattctacg |
| | agcgactgggtttcactgtgacagcagacgtagaagtccctgaagggcctcggac |
| | ttggtgcatgaccagaaagcctggggcttgaAACGCCATgatccagacatgataa |
| | gatacattgatgagtttggacaaaccacaactagaatgcagtgaGGCGCGCCTGG |
| | ATACTTCTGCCTGCAGGGGAGTCGCtcttttgcaatgctctttgggaaattatat |
| | tagcctaattactaatttcctggccctcaaggtgacttgcttgaacttgccacct |
| | TCGCCACTAGGGGGCAGCAttggtttacacagggtgaacaaacgttcaattctaa |
| | actaatattctttggtgggaaagtgtgttcattttggtttgttttgaagaagtca |
| | cttcatacctttgaactggggttaagctggttaactcccaaaattccaccagttc |
| | caatatcctatggaaaaaccccaaaaccactCAAAccagatatactcgttgacat |
| | tgattattgactagttattaatagtaatcaattacggggtcattagttcatagcc |
| | catatatggagttccgcgttacataacttacggtaaatggcccgcctggctgacc |
| | gcccaacgacccccgcccattgacgtcaataatgacgtatgttcccatagtaacg |
| | ccaatagggactttccattgacgtcaatgggtggagtatttacggtaaactgccc |
| | acttggcagtacatcaagtgtatcatatgccaagtacgccccctattgacgtcaa |
| | tgacggtaaatggcccgcctggcattatgcccagtacatgaccttatgggacttt |
| | cctacttggcagtacatctacgtattagtcatcgctattaccatggtgatgcggt |
| | tttggcagtacatcaatgggcgtggatagcggtttgactcacggggatttccaag |
| | tctccaccccattgacgtcaatgggagtttgttttggcaccaaaatcaacgggac |
| | tttccaaaatgtcgtaacaactccgccccattgacgcaaatgggcggtaggcgtg |
| | tacggtgggaggtctatataagcagagctctctggctaactagagaacccactgc |
| | ttactggcttatcgaaattaatacgactcactatagggagaaccaagcGAATGCC |
| | GCCACCATGGCTgtgagcaagggcgaggagctgttcaccggggtggtgcccatcc |
| | tggtcgagctggacggcgacgtaaacggccacaagttcagcgtgtccggcgaggg |
| | cgagggcgatgccacctacggcaagctgaccctgaagttcatctgcaccaccggc |
| | aagctgcccgtgccctggcccaccctcgtgaccaccctgacctacggcgtgcagt |
| | gcttcagccgctaccccgaccacatgaagcagcacgacttcttcaagtccgccat |
| | gcccgaaggctacgtccaggagcgcaccatcttcttcaaggacgacggcaactac |
| | aagacccgcgccgaggtgaagttcgagggcgacaccctggtgaaccgcatcgagc |
| | tgaagggcatcgacttcaaggaggacggcaacatcctggggcacaagctggagta |
| | caactacaacagccacaacgtctatatcatggccgacaagcagaagaacggcatc |
| | aaggtgaacttcaagatccgccacaacatcgaggacggcagcgtgcagctcgccg |
| | accactaccagcagaacacccccatcggcgacggccccgtgctgctgcccgacaa |
| | ccactacctgagcacccagtccgccctgagcaaagaccccaacgagaagcgcgat |
| | cacatggtcctgctggagttcgtgaccgccgccgggatcactctcggcatggacg |
| | agctgtacaagtaaAACGCCATgaattcactcctcaggtgcaggctgcctatcag |
| | aaggtggtggctggtgtggccaatgccctggctcacaaataccactgagatcttt |
| | ttccctctgccaaaaattatggggacatcatgaagccccttgagcatctgacttc |
| | tggctaataaaggaaatttattttcattgcaatagtgtgttggaattttttgtgt |
| | ctctcactcggaaggacatatgggagggcaaatcatttaaaacatcagaatgagt |
| | atttggtttagagtttggcaacatatgcccatatgctggctgccatgaacaaagg |
| | ttggctataaagaggtcatcagtatatgaaacagccccctgctgtccattcctta |
| | ttccatagaaaagccttgacttgaggttagattttttttatattttgttttgtgt |
| | tatttttttctttaacatccctaaaattttccttacatgttttactagccagatt |
| | tttcctcctctcctgactactcccagtcatagctgtccctcttctcttatggaga |
| | tcCAGTGGCATCGCtcttttgcaatgctctttgggaaattatattagcctaatta |
| | ctaatttcctggccctcaaggtgacttgcttgaacttgccacctTCGCCACTAGG |
| | GGGCAGCAttggtttacacagggtgaacaaacgttcaattctaaactaatattct |
| | ttggtgggaaagtgtgttcattttggtttgttttgaagaagtcacttcatacctt |
| | tgaactggggttaagctggttaactcccaaaattccaccagttccaatatcctat |
| | ggaaaaaccccaaaaccactCAAAccagatatactcgttgacattgattattgac |
| | tagttattaatagtaatcaattacggggtcattagttcatagcccatatatggag |
| | ttccgcgttacataacttacggtaaatggcccgcctggctgaccgcccaacgacc |
| | cccgcccattgacgtcaataatgacgtatgttcccatagtaacgccaatagggac |
| | tttccattgacgtcaatgggtggagtatttacggtaaactgcccacttggcagta |
| | catcaagtgtatcatatgccaagtacgccccctattgacgtcaatgacggtaaat |
| | ggcccgcctggcattatgcccagtacatgaccttatgggactttcctacttggca |
| | gtacatctacgtattagtcatcgctattaccatggtgatgcggttttggcagtac |
| | atcaatgggcgtggatagcggtttgactcacggggatttccaagtctccacccca |
| | ttgacgtcaatgggagtttgttttggcaccaaaatcaacgggactttccaaaatg |
| | tcgtaacaactccgccccattgacgcaaatgggcggtaggcgtgtacggtgggag |
| | gtctatataagcagagctctctggctaactagagaacccactgcttactggctta |
| | tcgaaattaatacgactcactatagggagaaccaagcGAATGCCGCCACCATGGC |
| | TGTCAGTAAAGGGGAAgagcttataaaggaaaatatgcacatgaagctctacatg |
| | gagggcactgtagataaccaccatttcaaatgtacctctgaaggggagggcaagc |
| | catacgaaggtactcaaaccatgcgaataaaagtagttgaaggcgggcctcttcc |
| | ctttgcattcgacattctcgcaacctcttttctgtacggcagtaagactttcata |
| | aaccacactcaaggcattccagacttcttcaagcaatcattccccgaaggattca |
| | cctgggagcgagttactacttatgaggacggaggagtccttactgcaacccaaga |
| | cacctcactgcaagatgggtgcctgatttacaatgtaaagatcagaggggtgaat |
| | ttcacaagcaatgggccagttatgcaaaaaaagacccttggatgggaggccttca |
| | ccgagacactgtacccagccgatggtggactggagggcaggaatgacatggccct |
| | caagctcgtcggaggcagtcacttgattgccaacgccaaaaccacttaccgctct |
| | aagaaacctgctaaaaacctgaagatgcccggcgtctattatgtggactatcgac |
| | ttgagagaattaaggaggcaaacaacgagacttatgtcgaacagcatgaagttgc |
| | cgtggctaggtactgtgatttgcccagcaaattgggtcataaacttaacTGAAAC |
| | GCCATgaattcactcctcaggtgcaggctgcctatcagaaggtggtggctggtgt |
| | ggccaatgccctggctcacaaataccactgagatctttttccctctgccaaaaat |
| | tatggggacatcatgaagccccttgagcatctgacttctggctaataaaggaaat |
| | ttattttcattgcaatagtgtgttggaattttttgtgtctctcactcggaaggac |
| | atatgggagggcaaatcatttaaaacatcagaatgagtatttggtttagagtttg |
| | gcaacatatgcccatatgctggctgccatgaacaaaggttggctataaagaggtc |
| | atcagtatatgaaacagccccctgctgtccattccttattccatagaaaagcctt |
| | gacttgaggttagattttttttatattttgttttgtgttatttttttctttaaca |
| | tccctaaaattttccttacatgttttactagccagatttttcctcctctcctgac |
| | tactcccagtcatagctgtccctcttctcttatggagatcCAGTTCGCGCGGCCG |
| | CGCCTGGGCTTACttaattaacgagagcataatattgatatgtgccaaagttgtt |
| | tctgactgactaataagtataatttgtttctattatgtataggttaagctaatta |
| | cttattttataatacaacatgactgtttttaaagtacaaaataagtttatttttg |
| | taaaagagagaatgtttaaaagttttgttactttatagaagaaattttgagtttt |
| | tgtttttttttaataaataaataaacataaataaattgtttgttgaatttattat |
| | tagtatgtaagtgtaaatataataaaacttaatatctattcaaattaataaataa |
| | acctcgatatacagaccgataaaacacatgcgtcaattttacgcatgattatctt |
| | taacgtacgtcacaatatgattatctttctagggttaaataatagtttctaattt |
| | ttttattattcagcctgctgtcgtgaataccgagctccaattcgccctatagtga |
| | gtcgtattacaattcactggccgtcgttttacaacgtcgtgactgggaaaaccct |
| | ggcgttacccaacttaatcgccttgcagcacatccccctttcgccagctggcgta |
| | atagcgaagaggcccgcaccgatcgcccttcccaacagttgcgcagcctgaatgg |
| | cgaatgggacgcgccctgtagcggcgcattaagcgcggcgggtgtggtggttacg |
| | cgcagcgtgaccgctacacttgccagcgccctagcgcccgctcctttcgctttct |
| | tcccttcctttctcgccacgttcgccggctttccccgtcaagctctaaatcgggg |
| | gctccctttagggttccgatttagtgctttacggcacctcgaccccaaaaaactt |
| | gattagggtgatggttcacgtagtgggccatcgccctgatagacggtttttcgcc |
| | ctttgacgttggagtccacgttctttaatagtggactcttgttccaaactggaac |
| | aacactcaaccctatctcggtctattcttttgatttataagggattttgccgatt |
| | tcggcctattggttaaaaaatgagctgatttaacaaaaatttaacgcgaatttta |
| | acaaaatattaacgcttacaatttaggtggcacttttcggggaaatgtgcgcgga |
| | acccctatttgtttatttttctaaatacattcaaatatgtatccgctcatgagac |
| | aataaccctgataaatgcttcaataatattgaaaaaggaagagtatgagtattca |
| | acatttccgtgtcgcccttattcccttttttgcggcattttgccttcctgttttt |
| | gctcacccagaaacgctggtgaaagtaaaagatgctgaagatcagttgggtgcac |
| | gagtgggttacatcgaactggatctcaacagcggtaagatccttgagagttttcg |
| | ccccgaagaacgttttccaatgatgagcacttttaaagttctgctatgtggcgcg |
| | gtattatcccgtattgacgccgggcaagagcaactcggtcgccgcatacactatt |
| | ctcagaatgacttggttgagtactcaccagtcacagaaaagcatcttacggatgg |
| | catgacagtaagagaattatgcagtgctgccataaccatgagtgataacactgcg |
| | gccaacttacttctgacaacgatcggaggaccgaaggagctaaccgcttttttgc |
| | acaacatgggggatcatgtaactcgccttgatcgttgggaaccggagctgaatga |
| | agccataccaaacgacgagcgtgacaccacgatgcctgtagcaatggcaacaacg |
| | ttgcgcaaactattaactggcgaactacttactctagcttcccggcaacaattaa |
| | tagactggatggaggcggataaagttg |
| | (SEQ ID NO: 23) |
| pAJW_P30 | tgacgccgggcaagagcaactcggtcgccgcatacactattctcagaatgacttg |
| | gttgagtactcaccagtcacagaaaagcatcttacggatggcatgacagtaagag |
| | aattatgcagtgctgccataaccatgagtgataacactgcggccaacttacttct |
| | gacaacgatcggaggaccgaaggagctaaccgcttttttgcacaacatgggggat |
| | catgtaactcgccttgatcgttgggaaccggagctgaatgaagccataccaaacg |
| | acgagcgtgacaccacgatgcctgtagcaatggcaacaacgttgcgcaaactatt |
| | aactggcgaactacttactctagcttcccggcaacaattaatagactggatggag |
| | gcggataaagttgcaggaccacttctgcgctcggcccttccggctggctggttta |
| | ttgctgataaatctggagccggtgagcgtgggtctcgcggtatcattgcagcact |
| | ggggccagatggtaagccctcccgtatcgtagttatctacacgacggggagtcag |
| | gcaactatggatgaacgaaatagacagatcgctgagataggtgcctcactgatta |
| | agcattggtaactgtcagaccaagtttactcatatatactttagattgatttaaa |
| | acttcatttttaatttaaaaggatctaggtgaagatcctttttgataatctcatg |
| | accaaaatcccttaacgtgagttttcgttccactgagcgtcagaccccgtagaaa |
| | agatcaaaggatcttcttgagatcctttttttctgcgcgtaatctgctgcttgca |
| | aacaaaaaaaccaccgctaccagcggtggtttgtttgccggatcaagagctacca |
| | actctttttccgaaggtaactggcttcagcagagcgcagataccaaatactgttc |
| | ttctagtgtagccgtagttaggccaccacttcaagaactctgtagcaccgcctac |
| | atacctcgctctgctaatcctgttaccagtggctgctgccagtggcgataagtcg |
| | tgtcttaccgggttggactcaagacgatagttaccggataaggcgcagcggtcgg |
| | gctgaacggggggttcgtgcacacagcccagcttggagcgaacgacctacaccga |
| | actgagatacctacagcgtgagctatgagaaagcgccacgcttcccgaagggaga |
| | aaggggacaggtatccggtaagcggcagggtcggaacaggagagcgcacgaggga |
| | gcttccagggggaaacgcctggtatctttatagtcctgtcgggtttcgccacctc |
| | tgacttgagcgtcgatttttgtgatgctcgtcaggggggcggagcctatggaaaa |
| | acgccagcaacgcggcctttttacggttcctggccttttgctggccttttgctca |
| | catgttctttcctgcgttatcccctgattctgtggataaccgtattaccgccttt |
| | gagtgagctgataccgctcgccgcagccgaacgaccgagcgcagcgagtcagtga |
| | gcgaggaagcggaagagcgcccaatacgcaaaccgcctctccccgcgcgttggcc |
| | gattcattaatgcagctggcacgacaggtttcccgactggaaagcgggcagtgag |
| | cgcaacgcaattaatgtgagttagctcactcattaggcaccccaggctttacact |
| | ttatgcttccggctcgtatgttgtgtggaattgtgagcggataacaatttcacac |
| | aggaaacagctatgaccatgattacgccaagctcgaaattaaccctcactaaagg |
| | gaacaaaagctggtacctcgcgcgacttggtttgccattctttagcgcgcgtcgc |
| | gtcacacagcttggccacaatgtggtttttgtcaaacgaagattctatgacgtgt |
| | ttaaagtttaggtcgagtaaagcgcaaatcttttttaaccctagaaagatagtct |
| | gcgtaaaattgacgcatgcattcttgaaatattgctctctctttctaaatagcgc |
| | gaatccgtcgctgtgcatttaggacatctcagtcgccgcttggagctcccgtgag |
| | gcgtgcttgtcaatgcggtaagtgtcactgattttgaactataacgaccgcgtga |
| | gtcaaaatgacgcatgattatcttttacgtgacttttaagatttaactcatacga |
| | taattatattgttatttcatgttctacttacgtgataacttattatatatatatt |
| | ttcttgttatagatatcatcaactttgtatagaaaagttgctcgacattgattat |
| | tgactagttattaatagtaatcaattacggggtcattagttcatagcccatatat |
| | ggagttccgcgttacataacttacggtaaatggcccgcctggctgaccgcccaac |
| | gacccccgcccattgacgtcaataatgacgtatgttcccatagtaacgccaatag |
| | ggactttccattgacgtcaatgggtggagtatttacggtaaactgcccacttggc |
| | agtacatcaagtgtatcatatgccaagtacgccccctattgacgtcaatgacggt |
| | aaatggcccgcctggcattatgcccagtacatgaccttatgggactttcctactt |
| | ggcagtacatctacgtattagtcatcgctattaccatggtcgaggtgagccccac |
| | gttctgcttcactctccccatctcccccccctccccacccccaattttgtattta |
| | tttattttttaattattttgtgcagcgatgggggcggggggggggggggggcgcg |
| | cgccaggcggggcggggcggggcgaggggcggggcggggcgaggcggagaggtgc |
| | ggcggcagccaatcagagcggcgcgctccgaaagtttccttttatggcgaggcgg |
| | cggcggcggcggccctataaaaagcgaagcgcgcggggggggagtcgctgcgcgc |
| | tgccttcgccccgtgccccgctccgccgccgcctcgcgccgcccgccccggctct |
| | gactgaccgcgttactcccacaggtgagcgggcgggacggcccttctcctccggg |
| | ctgtaattagcgcttggtttaatgacggcttgtttcttttctgtggctgcgtgaa |
| | agccttgaggggctccgggagggccctttgtgcggggggagcggctcggggggtg |
| | cgtgcgtgtgtgtgtgcgtggggagcgccgcgtgcggctccgcgctgcccggcgg |
| | ctgtgagcgctgcgggcgcggcgcggggctttgtgcgctccgcagtgtgcgcgag |
| | gggagcgcggccgggggcggtgccccgcggtgcggggggggctgcgaggggaaca |
| | aaggctgcgtgcggggtgtgtgcgtgggggggtgagcagggggtgtgggcgcgtc |
| | ggtcgggctgcaaccccccctgcacccccctccccgagttgctgagcacggcccg |
| | gcttcgggtgcggggctccgtacggggcgtggcgcggggctcgccgtgccgggcg |
| | gggggtggcggcaggtgggggtgccgggcggggcggggccgcctcgggccgggga |
| | gggctcgggggaggggcgcggcggcccccggagcgccggcggctgtcgaggcgcg |
| | gcgagccgcagccattgccttttatggtaatcgtgcgagagggcgcagggacttc |
| | ctttgtcccaaatctgtgcggagccgaaatctgggaggcgccgccgcaccccctc |
| | tagcgggcgcggggcgaagcggtgcggcgccggcaggaaggaaatgggcggggag |
| | ggccttcgtgcgtcgccgcgccgccgtccccttctccctctccagcctcggggct |
| | gtccgcggggggacggctgccttcgggggggacggggcagggcggggttcggctt |
| | ctggcgtgtgaccggcggctctagagcctctgctaaccatgttcatgccttcttc |
| | tttttcctacagctcctgggcaacgtgctggttattgtgctgtctcatcattttg |
| | gcaaagaattgcaagtttgtacaaaaaagcaggctGAATGCCGCCACCATGGCTa |
| | ccgagtataaaccgacggtccgacttgcgactcgggatgacgttccacgcgccgt |
| | gaggactctggccgccgccttcgctgactacccggccacacggcacacagttgac |
| | cccgacagacacatagagcgggtcacagagttgcaagaacttttcctgacgcggg |
| | ttggActcgatattggaaaagtttgggttgccgacgatggtgccgcagttgcggt |
| | gtggactactccagaaagtgtagaagctggagccgtattcgctgaaatagggcca |
| | cggatggcagagctgtctggtagcaggctcgcagcgcagcagcagatggaaggCc |
| | ttctcgctccgcatcggcctaaggaaccggcgtggttcctggctacggtgggagt |
| | ctcacctgaccatcaaggaaagggattgggaagtgctgtcgttcttccaggcgtg |
| | gaagcggccgaacgagcaggcgtcccagccttcttggaAacgagcgcacccagga |
| | atcttccattctacgagcgactgggtttcactgtgacagcagacgtagaagtccc |
| | tgaagggcctcggacttggtgcatgaccagaaagcctggggcttgaAACGCCATg |
| | atccagacatgataagatacattgatgagtttggacaaaccacaactagaatgca |
| | gtgaGGCGCGCCTGGATACTTCTGCCTGCAGGGGAGTCGCtcttttgcaatgctc |
| | tttgggaaattatattagcctaattactaatttcctggccctcaaggtgacttgc |
| | ttgaacttgccacctTCGCCACTAGGGGGCAGCAttggtttacacagggtgaaca |
| | aacgttcaattctaaactaatattctttggtgggaaagtgtgttcattttggttt |
| | gttttgaagaagtcacttcatacctttgaactggggttaagctggttaactccca |
| | aaattccaccagttccaatatcctatggaaaaaccccaaaaccactCAAAccaga |
| | tatactcgttgacattgattattgactagttattaatagtaatcaattacggggt |
| | cattagttcatagcccatatatggagttccgcgttacataacttacggtaaatgg |
| | cccgcctggctgaccgcccaacgacccccgcccattgacgtcaataatgacgtat |
| | gttcccatagtaacgccaatagggactttccattgacgtcaatgggtggagtatt |
| | tacggtaaactgcccacttggcagtacatcaagtgtatcatatgccaagtacgcc |
| | ccctattgacgtcaatgacggtaaatggcccgcctggcattatgcccagtacatg |
| | accttatgggactttcctacttggcagtacatctacgtattagtcatcgctatta |
| | ccatggtgatgcggttttggcagtacatcaatgggcgtggatagcggtttgactc |
| | acggggatttccaagtctccaccccattgacgtcaatgggagtttgttttggcac |
| | caaaatcaacgggactttccaaaatgtcgtaacaactccgccccattgacgcaaa |
| | tgggcggtaggcgtgtacggtgggaggtctatataagcagagctctctggctaac |
| | tagagaacccactgcttactggcttatcgaaattaatacgactcactatagggag |
| | aaccaagcGAATGCCGCCACCATGGCTATGgtgagcaagggcgaggagctgttca |
| | ccggggtggtgcccatcctggtcgagctggacggcgacgtaaacggccacaagtt |
| | cagcgtgtccggcgagggcgagggcgatgccacctacggcaagctgaccctgaag |
| | ttcatctgcaccaccggcaagctgcccgtgccctggcccaccctcgtgaccaccc |
| | tgacctacggcgtgcagtgcttcagccgctaccccgaccacatgaagcagcacga |
| | cttcttcaagtccgccatgcccgaaggctacgtccaggagcgcaccatcttcttc |
| | aaggacgacggcaactacaagacccgcgccgaggtgaagttcgagggcgacaccc |
| | tggtgaaccgcatcgagctgaagggcatcgacttcaaggaggacggcaacatcct |
| | ggggcacaagctggagtacaactacaacagccacaacgtctatatcatggccgac |
| | aagcagaagaacggcatcaaggtgaacttcaagatccgccacaacatcgaggacg |
| | gcagcgtgcagctcgccgaccactaccagcagaacacccccatcggcgacggccc |
| | cgtgctgctgcccgacaaccactacctgagcacccagtccgccctgagcaaagac |
| | cccaacgagaagcgcgatcacatggtcctgctggagttcgtgaccgccgccggga |
| | tcactctcggcatggacgagctgtacaagtaaAACGCCATgaattcactcctcag |
| | gtgcaggctgcctatcagaaggtggtggctggtgtggccaatgccctggctcaca |
| | aataccactgagatctttttccctctgccaaaaattatggggacatcatgaagcc |
| | ccttgagcatctgacttctggctaataaaggaaatttattttcattgcaatagtg |
| | tgttggaattttttgtgtctctcactcggaaggacatatgggagggcaaatcatt |
| | taaaacatcagaatgagtatttggtttagagtttggcaacatatgcccatatgct |
| | ggctgccatgaacaaaggttggctataaagaggtcatcagtatatgaaacagccc |
| | cctgctgtccattccttattccatagaaaagccttgacttgaggttagatttttt |
| | ttatattttgttttgtgttatttttttctttaacatccctaaaattttccttaca |
| | tgttttactagccagatttttcctcctctcctgactactcccagtcatagctgtc |
| | cctcttctcttatggagatCAGTGGCATCGCtcttttgcaatgctctttgggaaa |
| | ttatattagcctaattactaatttcctggccctcaaggtgacttgcttgaacttg |
| | ccacctTCGCCACTAGGGGGCAGCAttggtttacacagggtgaacaaacgttcaa |
| | ttctaaactaatattctttggtgggaaagtgtgttcattttggtttgttttgaag |
| | aagtcacttcatacctttgaactggggttaagctggttaactcccaaaattccac |
| | cagttccaatatcctatggaaaaaccccaaaaccactCAAAccagatatactcgt |
| | tgacattgattattgactagttattaatagtaatcaattacggggtcattagttc |
| | atagcccatatatggagttccgcgttacataacttacggtaaatggcccgcctgg |
| | ctgaccgcccaacgacccccgcccattgacgtcaataatgacgtatgttcccata |
| | gtaacgccaatagggactttccattgacgtcaatgggtggagtatttacggtaaa |
| | ctgcccacttggcagtacatcaagtgtatcatatgccaagtacgccccctattga |
| | cgtcaatgacggtaaatggcccgcctggcattatgcccagtacatgaccttatgg |
| | gactttcctacttggcagtacatctacgtattagtcatcgctattaccatggtga |
| | tgcggttttggcagtacatcaatgggcgtggatagcggtttgactcacggggatt |
| | tccaagtctccaccccattgacgtcaatgggagtttgttttggcaccaaaatcaa |
| | cgggactttccaaaatgtcgtaacaactccgccccattgacgcaaatgggcggta |
| | ggcgtgtacggtgggaggtctatataagcagagctctctggctaactagagaacc |
| | cactgcttactggcttatcgaaattaatacgactcactatagggagaaccaagcG |
| | AATGCCGCCACCATGGCTgctgaaggTtccgtcgccaggcagcctgacctcttga |
| | cctgcgacgatgagccgatccatatccccggtgccatccaaccgcatggactgct |
| | gctcgccctcgccgccgacatgacgatcgttgccggcagcgacaaccttcccgaa |
| | ctcaccggactggcgatcggcgccctgatcggccgctctgcggccgatgtGttcg |
| | actcggaAacgcacaaccgtctgacgatcgccttggccgagcccggggcggccgt |
| | cggagcaccgatcactgtcggcttcacgatgcgaaaggacgcaggcttcatcggc |
| | tcctggcatcgccatgatcagctcatcttcctGgagctGgagcctccccagcggg |
| | acgtcgccgagccgcaggcgttcttccgccgcaccaacagcgccatccgccgcct |
| | gcaggccgccgaaaccttggaaagcgcctgcgccgccgcggcgcaagaggtgcgg |
| | aagattaccggcttcgatcgggtgatgatctatcgcttcgcctccgacttcagcg |
| | gcgaagtgatcgcagaggatcggtgcgccgaggtcgagtcaaaactaggcctgca |
| | ctatcctgcctcaaccgtgccggcgcaggcccgtcggctctataccatcaacccg |
| | gtacggatcattcccgatatcaattatcggccggtgccggtcaccccagacctca |
| | atccggtcaccgggcggccgattgatcttagcttcgccatcctgcgcagcgtGtc |
| | gcccgtccatctggaGttcatgcgcaacataggcatgcacggcacgatgtcgatc |
| | tcgattttgcgcggcgagcgactgtggggattgatcgtttgccatcaccgaacgc |
| | cgtactacgtcgatctcgatggccgccaagcctgcgagctagtcgcccaggttct |
| | ggcctggcagatcggcgtgatggaagagTAAAACGCCATgaattcactcctcagg |
| | tgcaggctgcctatcagaaggtggtggctggtgtggccaatgccctggctcacaa |
| | ataccactgagatctttttccctctgccaaaaattatggggacatcatgaagccc |
| | cttgagcatctgacttctggctaataaaggaaatttattttcattgcaatagtgt |
| | gttggaattttttgtgtctctcactcggaaggacatatgggagggcaaatcattt |
| | aaaacatcagaatgagtatttggtttagagtttggcaacatatgcccatatgctg |
| | gctgccatgaacaaaggttggctataaagaggtcatcagtatatgaaacagcccc |
| | ctgctgtccattccttattccatagaaaagccttgacttgaggttagattttttt |
| | tatattttgttttgtgttatttttttctttaacatccctaaaattttccttacat |
| | gttttactagccagatttttcctcctctcctgactactcccagtcatagctgtcc |
| | ctcttctcttatggagatCAGTTCGCGCGGCCGCGCCTGGGCTTACttaattaac |
| | gagagcataatattgatatgtgccaaagttgtttctgactgactaataagtataa |
| | tttgtttctattatgtataggttaagctaattacttattttataatacaacatga |
| | ctgtttttaaagtacaaaataagtttatttttgtaaaagagagaatgtttaaaag |
| | ttttgttactttatagaagaaattttgagtttttgtttttttttaataaataaat |
| | aaacataaataaattgtttgttgaatttattattagtatgtaagtgtaaatataa |
| | taaaacttaatatctattcaaattaataaataaacctcgatatacagaccgataa |
| | aacacatgcgtcaattttacgcatgattatctttaacgtacgtcacaatatgatt |
| | atctttctagggttaaataatagtttctaatttttttattattcagcctgctgtc |
| | gtgaataccgagctccaattcgccctatagtgagtcgtattacaattcactggcc |
| | gtcgttttacaacgtcgtgactgggaaaaccctggcgttacccaacttaatcgcc |
| | ttgcagcacatccccctttcgccagctggcgtaatagcgaagaggcccgcaccga |
| | tcgcccttcccaacagttgcgcagcctgaatggcgaatgggacgcgccctgtagc |
| | ggcgcattaagcgcggcgggtgtggtggttacgcgcagcgtgaccgctacacttg |
| | ccagcgccctagcgcccgctcctttcgctttcttcccttcctttctcgccacgtt |
| | cgccggctttccccgtcaagctctaaatcgggggctccctttagggttccgattt |
| | agtgctttacggcacctcgaccccaaaaaacttgattagggtgatggttcacgta |
| | gtgggccatcgccctgatagacggtttttcgccctttgacgttggagtccacgtt |
| | ctttaatagtggactcttgttccaaactggaacaacactcaaccctatctcggtc |
| | tattcttttgatttataagggattttgccgatttcggcctattggttaaaaaatg |
| | agctgatttaacaaaaatttaacgcgaattttaacaaaatattaacgcttacaat |
| | ttaggtggcacttttcggggaaatgtgcgcggaacccctatttgtttatttttct |
| | aaatacattcaaatatgtatccgctcatgagacaataaccctgataaatgcttca |
| | ataatattgaaaaaggaagagtatgagtattcaacatttccgtgtcgcccttatt |
| | cccttttttgcggcattttgccttcctgtttttgctcacccagaaacgctggtga |
| | aagtaaaagatgctgaagatcagttgggtgcacgagtgggttacatcgaactgga |
| | tctcaacagcggtaagatccttgagagttttcgccccgaagaacgttttccaatg |
| | atgagcacttttaaagttctgctatgtggcgcggtattatcccgtat |
| | (SEQ ID NO: 24) |
| pAJW_P31 | caggaccacttctgcgctcggcccttccggctggctggtttattgctgataaatc |
| | tggagccggtgagcgtgggtctcgcggtatcattgcagcactggggccagatggt |
| | aagccctcccgtatcgtagttatctacacgacggggagtcaggcaactatggatg |
| | aacgaaatagacagatcgctgagataggtgcctcactgattaagcattggtaact |
| | gtcagaccaagtttactcatatatactttagattgatttaaaacttcatttttaa |
| | tttaaaaggatctaggtgaagatcctttttgataatctcatgaccaaaatccctt |
| | aacgtgagttttcgttccactgagcgtcagaccccgtagaaaagatcaaaggatc |
| | ttcttgagatcctttttttctgcgcgtaatctgctgcttgcaaacaaaaaaacca |
| | ccgctaccagcggtggtttgtttgccggatcaagagctaccaactctttttccga |
| | aggtaactggcttcagcagagcgcagataccaaatactgttcttctagtgtagcc |
| | gtagttaggccaccacttcaagaactctgtagcaccgcctacatacctcgctctg |
| | ctaatcctgttaccagtggctgctgccagtggcgataagtcgtgtcttaccgggt |
| | tggactcaagacgatagttaccggataaggcgcagcggtcgggctgaacgggggg |
| | ttcgtgcacacagcccagcttggagcgaacgacctacaccgaactgagataccta |
| | cagcgtgagctatgagaaagcgccacgcttcccgaagggagaaaggcggacaggt |
| | atccggtaagcggcagggtcggaacaggagagcgcacgagggagcttccaggggg |
| | aaacgcctggtatctttatagtcctgtcgggtttcgccacctctgacttgagcgt |
| | cgatttttgtgatgctcgtcaggggggcggagcctatggaaaaacgccagcaacg |
| | cggcctttttacggttcctggccttttgctggccttttgctcacatgttctttcc |
| | tgcgttatcccctgattctgtggataaccgtattaccgcctttgagtgagctgat |
| | accgctcgccgcagccgaacgaccgagcgcagcgagtcagtgagcgaggaagcgg |
| | aagagcgcccaatacgcaaaccgcctctccccgcgcgttggccgattcattaatg |
| | cagctggcacgacaggtttcccgactggaaagcgggcagtgagcgcaacgcaatt |
| | aatgtgagttagctcactcattaggcaccccaggctttacactttatgcttccgg |
| | ctcgtatgttgtgtggaattgtgagcggataacaatttcacacaggaaacagcta |
| | tgaccatgattacgccaagctcgaaattaaccctcactaaagggaacaaaagctg |
| | gtacctcgcgcgacttggtttgccattctttagcgcgcgtcgcgtcacacagctt |
| | ggccacaatgtggtttttgtcaaacgaagattctatgacgtgtttaaagtttagg |
| | tcgagtaaagcgcaaatcttttttaaccctagaaagatagtctgcgtaaaattga |
| | cgcatgcattcttgaaatattgctctctctttctaaatagcgcgaatccgtcgct |
| | gtgcatttaggacatctcagtcgccgcttggagctcccgtgaggcgtgcttgtca |
| | atgcggtaagtgtcactgattttgaactataacgaccgcgtgagtcaaaatgacg |
| | catgattatcttttacgtgacttttaagatttaactcatacgataattatattgt |
| | tatttcatgttctacttacgtgataacttattatatatatattttcttgttatag |
| | atatcatcaactttgtatagaaaagttgctcgacattgattattgactagttatt |
| | aatagtaatcaattacggggtcattagttcatagcccatatatggagttccgcgt |
| | tacataacttacggtaaatggcccgcctggctgaccgcccaacgacccccgccca |
| | ttgacgtcaataatgacgtatgttcccatagtaacgccaatagggactttccatt |
| | gacgtcaatgggtggagtatttacggtaaactgcccacttggcagtacatcaagt |
| | gtatcatatgccaagtacgccccctattgacgtcaatgacggtaaatggcccgcc |
| | tggcattatgcccagtacatgaccttatgggactttcctacttggcagtacatct |
| | acgtattagtcatcgctattaccatggtcgaggtgagccccacgttctgcttcac |
| | tctccccatctcccccccctccccacccccaattttgtatttatttattttttaa |
| | ttattttgtgcagcgatgggggcggggggggggggggggcgcgcgccaggcgggg |
| | cggggcggggcgaggggcggggggggcgaggcggagaggtgcggcggcagccaat |
| | cagagcggcgcgctccgaaagtttccttttatggcgaggcggcggcggcggcggc |
| | cctataaaaagcgaagcgcgcggcggggggagtcgctgcgcgctgccttcgcccc |
| | gtgccccgctccgccgccgcctcgcgccgcccgccccggctctgactgaccgcgt |
| | tactcccacaggtgagcggggggacggcccttctcctccgggctgtaattagcgc |
| | ttggtttaatgacggcttgtttcttttctgtggctgcgtgaaagccttgaggggc |
| | tccgggagggcccgcgtgcggctccgcgctgcccggcggctgtgagcgctgcggg |
| | cgcggcgcggggctttgtgcgctccgcagtgtgcgcgaggggagcgcggccgggg |
| | gcggtgccccgcggtgcggggggggctgcgaggggaacaaaggctgcgtgcgggg |
| | tgtgtgcgtgggggggtgagcagggggtgtgggcgcgtcggtcgggctgcaaccc |
| | cccctgcacccccctccccgagttgctgagcacggcccggcttcgggtgcggggc |
| | tccgtacggggcgtggcgcggggctcgccgtgccgggcggggggtggcggcaggt |
| | gggggtgccgggcggggggggccgcctcgggccggggagggctcgggggaggggc |
| | gcggcggcccccggagcgccggcggctgtcgaggcgcggcgagccgcagccattg |
| | ccttttatggtaatcgtgcgagagggcgcagggacttcctttgtcccaaatctgt |
| | gcggagccgaaatctgggaggcgccgccgcaccccctctagcgggcgcggggcga |
| | agcggtgcggcgccggcaggaaggaaatgggcggggagggccttcgtgcgtcgcc |
| | gcgccgccgtccccttctccctctccagcctcggggctgtccgcggggggacggc |
| | tgccttcgggggggacggggcagggcggggttcggcttctggcgtgtgaccggcg |
| | gctctagagcctctgctaaccatgttcatgccttcttctttttcctacagctcct |
| | gggcaacgtgctggttattgtgctgtctcatcattttggcaaagaattgcaagtt |
| | tgtacaaaaaagcaggctGAATGCCGCCACCATGGCTaccgagtataaaccgacg |
| | gtccgacttgcgactcgggatgacgttccacgcgccgtgaggactctggccgccg |
| | ccttcgctgactacccggccacacggcacacagttgaccccgacagacacataga |
| | gcgggtcacagagttgcaagaacttttcctgacgcgggttggActcgatattgga |
| | aaagtttgggttgccgacgatggtgccgcagttgcggtgtggactactccagaaa |
| | gtgtagaagctggagccgtattcgctgaaatagggccacggatggcagagctgtc |
| | tggtagcaggctcgcagcgcagcagcagatggaaggCcttctcgctccgcatcgg |
| | cctaaggaaccggcgtggttcctggctacggtgggagtctcacctgaccatcaag |
| | gaaagggattgggaagtgctgtcgttcttccaggcgtggaagcggccgaacgagc |
| | aggcgtcccagccttcttggaAacgagcgcacccaggaatcttccattctacgag |
| | cgactgggtttcactgtgacagcagacgtagaagtccctgaagggcctcggactt |
| | ggtgcatgaccagaaagcctggggcttgaAACGCCATgatccagacatgataaga |
| | tacattgatgagtttggacaaaccacaactagaatgcagtgaGGCGCGCCTGGAT |
| | ACTTCTGCCTGCAGGGGAGTCGCtcttttgcaatgctctttgggaaattatatta |
| | gcctaattactaatttcctggccctcaaggtgacttgcttgaacttgccacctTC |
| | GCCACTAGGGGGCAGCAttggtttacacagggtgaacaaacgttcaattctaaac |
| | taatattctttggtgggaaagtgtgttcattttggtttgttttgaagaagtcact |
| | tcatacctttgaactggggttaagctggttaactcccaaaattccaccagttcca |
| | atatcctatggaaaaaccccaaaaccactCAAAccagatatactcgttgacattg |
| | attattgactagttattaatagtaatcaattacggggtcattagttcatagccca |
| | tatatggagttccgcgttacataacttacggtaaatggcccgcctggctgaccgc |
| | ccaacgacccccgcccattgacgtcaataatgacgtatgttcccatagtaacgcc |
| | aatagggactttccattgacgtcaatgggtggagtatttacggtaaactgcccac |
| | ttggcagtacatcaagtgtatcatatgccaagtacgccccctattgacgtcaatg |
| | acggtaaatggcccgcctggcattatgcccagtacatgaccttatgggactttcc |
| | tacttggcagtacatctacgtattagtcatcgctattaccatggtgatgcggttt |
| | tggcagtacatcaatgggcgtggatagcggtttgactcacggggatttccaagtc |
| | tccaccccattgacgtcaatgggagtttgttttggcaccaaaatcaacgggactt |
| | tccaaaatgtcgtaacaactccgccccattgacgcaaatgggcggtaggcgtgta |
| | cggtgggaggtctatataagcagagctctctggctaactagagaacccactgctt |
| | actggcttatcgaaattaatacgactcactatagggagaaccaagcGAATGCCGC |
| | CACCATGGCTGTCAGTAAAGGGGAAgagcttataaaggaaaatatgcacatgaag |
| | ctctacatggagggcactgtagataaccaccatttcaaatgtacctctgaagggg |
| | agggcaagccatacgaaggtactcaaaccatgcgaataaaagtagttgaaggcgg |
| | gcctcttccctttgcattcgacattctcgcaacctcttttctgtacggcagtaag |
| | actttcataaaccacactcaaggcattccagacttcttcaagcaatcattccccg |
| | aaggattcacctgggagcgagttactacttatgaggacggaggagtccttactgc |
| | aacccaagacacctcactgcaagatgggtgcctgatttacaatgtaaagatcaga |
| | ggggtgaatttcacaagcaatgggccagttatgcaaaaaaagacccttggatggg |
| | aggccttcaccgagacactgtacccagccgatggtggactggagggcaggaatga |
| | catggccctcaagctcgtcggaggcagtcacttgattgccaacgccaaaaccact |
| | taccgctctaagaaacctgctaaaaacctgaagatgcccggcgtctattatgtgg |
| | actatcgacttgagagaattaaggaggcaaacaacgagacttatgtcgaacagca |
| | tgaagttgccgtggctaggtactgtgatttgcccagcaaattgggtcataaactt |
| | aacAACGCCATgaattcactcctcaggtgcaggctgcctatcagaaggtggtggc |
| | tggtgtggccaatgccctggctcacaaataccactgagatctttttccctctgcc |
| | aaaaattatggggacatcatgaagccccttgagcatctgacttctggctaataaa |
| | ggaaatttattttcattgcaatagtgtgttggaattttttgtgtctctcactcgg |
| | aaggacatatgggagggcaaatcatttaaaacatcagaatgagtatttggtttag |
| | agtttggcaacatatgcccatatgctggctgccatgaacaaaggttggctataaa |
| | gaggtcatcagtatatgaaacagccccctgctgtccattccttattccatagaaa |
| | agccttgacttgaggttagattttttttatattttgttttgtgttatttttttct |
| | ttaacatccctaaaattttccttacatgttttactagccagatttttcctcctct |
| | cctgactactcccagtcatagctgtccctcttctcttatggagatcCAGTGGCAT |
| | CGCtcttttgcaatgctctttgggaaattatattagcctaattactaatttcctg |
| | gccctcaaggtgacttgcttgaacttgccacctTCGCCACTAGGGGGCAGCAttg |
| | gtttacacagggtgaacaaacgttcaattctaaactaatattctttggtgggaaa |
| | gtgtgttcattttggtttgttttgaagaagtcacttcatacctttgaactggggt |
| | taagctggttaactcccaaaattccaccagttccaatatcctatggaaaaacccc |
| | aaaaccactCAAAccagatatactcgttgacattgattattgactagttattaat |
| | agtaatcaattacggggtcattagttcatagcccatatatggagttccgcgttac |
| | ataacttacggtaaatggcccgcctggctgaccgcccaacgacccccgcccattg |
| | acgtcaataatgacgtatgttcccatagtaacgccaatagggactttccattgac |
| | gtcaatgggtggagtatttacggtaaactgcccacttggcagtacatcaagtgta |
| | tcatatgccaagtacgccccctattgacgtcaatgacggtaaatggcccgcctgg |
| | cattatgcccagtacatgaccttatgggactttcctacttggcagtacatctacg |
| | tattagtcatcgctattaccatggtgatgcggttttggcagtacatcaatgggcg |
| | tggatagcggtttgactcacggggatttccaagtctccaccccattgacgtcaat |
| | gggagtttgttttggcaccaaaatcaacgggactttccaaaatgtcgtaacaact |
| | ccgccccattgacgcaaatgggcggtaggcgtgtacggtgggaggtctatataag |
| | cagagctctctggctaactagagaacccactgcttactggcttatcgaaattaat |
| | acgactcactatagggagaaccaagcGAATGCCGCCACCATGGCTGTCAGTAAAG |
| | GGGAAgagcttataaaggaaaatatgcacatgaagctctacatggagggcactgt |
| | agataaccaccatttcaaatgtacctctgaaggggagggcaagccatacgaaggt |
| | actcaaaccatgcgaataaaagtagttgaaggcgggcctcttccctttgcattcg |
| | acattctcgcaacctcttttctgtacggcagtaagactttcataaaccacactca |
| | aggcattccagacttcttcaagcaatcattccccgaaggattcacctgggagcga |
| | gttactacttatgaggacggaggagtccttactgcaacccaagacacctcactgc |
| | aagatgggtgcctgatttacaatgtaaagatcagaggggtgaatttcacaagcaa |
| | tgggccagttatgcaaaaaaagacccttggatgggaggccttcaccgagacactg |
| | tacccagccgatggtggactggagggcaggaatgacatggccctcaagctcgtcg |
| | gaggcagtcacttgattgccaacgccaaaaccacttaccgctctaagaaacctgc |
| | taaaaacctgaagatgcccggcgtctattatgtggactatcgacttgagagaatt |
| | aaggaggcaaacaacgagacttatgtcgaacagcatgaagttgccgtggctaggt |
| | actgtgatttgcccagcaaattgggtcataaacttaacTGAAACGCCATgaattc |
| | actcctcaggtgcaggctgcctatcagaaggtggtggctggtgtggccaatgccc |
| | tggctcacaaataccactgagatctttttccctctgccaaaaattatggggacat |
| | catgaagccccttgagcatctgacttctggctaataaaggaaatttattttcatt |
| | gcaatagtgtgttggaattttttgtgtctctcactcggaaggacatatgggaggg |
| | caaatcatttaaaacatcagaatgagtatttggtttagagtttggcaacatatgc |
| | ccatatgctggctgccatgaacaaaggttggctataaagaggtcatcagtatatg |
| | aaacagccccctgctgtccattccttattccatagaaaagccttgacttgaggtt |
| | agattttttttatattttgttttgtgttatttttttctttaacatccctaaaatt |
| | ttccttacatgttttactagccagatttttcctcctctcctgactactcccagtc |
| | atagctgtccctcttctcttatggagatcCAGTTCGCGCGGCCGCGCCTGGGCTT |
| | ACttaattaacgagagcataatattgatatgtgccaaagttgtttctgactgact |
| | aataagtataatttgtttctattatgtataggttaagctaattacttattttata |
| | atacaacatgactgtttttaaagtacaaaataagtttatttttgtaaaagagaga |
| | atgtttaaaagttttgttactttatagaagaaattttgagtttttgttttttttt |
| | aataaataaataaacataaataaattgtttgttgaatttattattagtatgtaag |
| | tgtaaatataataaaacttaatatctattcaaattaataaataaacctcgatata |
| | cagaccgataaaacacatgcgtcaattttacgcatgattatctttaacgtacgtc |
| | acaatatgattatctttctagggttaaataatagtttctaatttttttattattc |
| | agcctgctgtcgtgaataccgagctccaattcgccctatagtgagtcgtattaca |
| | attcactggccgtcgttttacaacgtcgtgactgggaaaaccctggcgttaccca |
| | acttaatcgccttgcagcacatccccctttcgccagctggcgtaatagcgaagag |
| | gcccgcaccgatcgcccttcccaacagttgcgcagcctgaatggcgaatgggacg |
| | cgccctgtagcggcgcattaagcgcggcgggtgtggtggttacgcgcagcgtgac |
| | cgctacacttgccagcgccctagcgcccgctcctttcgctttcttcccttccttt |
| | ctcgccacgttcgccggctttccccgtcaagctctaaatcgggggctccctttag |
| | ggttccgatttagtgctttacggcacctcgaccccaaaaaacttgattagggtga |
| | tggttcacgtagtgggccatcgccctgatagacggtttttcgccctttgacgttg |
| | gagtccacgttctttaatagtggactcttgttccaaactggaacaacactcaacc |
| | ctatctcggtctattcttttgatttataagggattttgccgatttcggcctattg |
| | gttaaaaaatgagctgatttaacaaaaatttaacgcgaattttaacaaaatatta |
| | acgcttacaatttaggtggcacttttcggggaaatgtgcgcggaacccctatttg |
| | tttatttttctaaatacattcaaatatgtatccgctcatgagacaataaccctga |
| | taaatgcttcaataatattgaaaaaggaagagtatgagtattcaacatttccgtg |
| | tcgcccttattcccttttttgcggcattttgccttcctgtttttgctcacccaga |
| | aacgctggtgaaagtaaaagatgctgaagatcagttgggtgcacgagtgggttac |
| | atcgaactggatctcaacagcggtaagatccttgagagttttcgccccgaagaac |
| | gttttccaatgatgagcacttttaaagttctgctatgtggcgcggtattatcccg |
| | tattgacgccgggcaagagcaactcggtcgccgcatacactattctcagaatgac |
| | ttggttgagtactcaccagtcacagaaaagcatcttacggatggcatgacagtaa |
| | gagaattatgcagtgctgccataaccatgagtgataacactgcggccaacttact |
| | tctgacaacgatcggaggaccgaaggagctaaccgcttttttgcacaacatgggg |
| | gatcatgtaactcgccttgatcgttgggaaccggagctgaatgaagccataccaa |
| | acgacgagcgtgacaccacgatgcctgtagcaatggcaacaacgttgcgcaaact |
| | attaactggcgaactacttactctagcttcccggcaacaattaatagactggatg |
| | gaggcggataaagttg |
| | (SEQ ID NO: 25) |
| pAJW_P32 | caggaccacttctgcgctcggcccttccggctggctggtttattgctgataaatc |
| | tggagccggtgagcgtgggtctcgcggtatcattgcagcactggggccagatggt |
| | aagccctcccgtatcgtagttatctacacgacggggagtcaggcaactatggatg |
| | aacgaaatagacagatcgctgagataggtgcctcactgattaagcattggtaact |
| | gtcagaccaagtttactcatatatactttagattgatttaaaacttcatttttaa |
| | tttaaaaggatctaggtgaagatcctttttgataatctcatgaccaaaatccctt |
| | aacgtgagttttcgttccactgagcgtcagaccccgtagaaaagatcaaaggatc |
| | ttcttgagatcctttttttctgcgcgtaatctgctgcttgcaaacaaaaaaacca |
| | ccgctaccagcggtggtttgtttgccggatcaagagctaccaactctttttccga |
| | aggtaactggcttcagcagagcgcagataccaaatactgttcttctagtgtagcc |
| | gtagttaggccaccacttcaagaactctgtagcaccgcctacatacctcgctctg |
| | ctaatcctgttaccagtggctgctgccagtggcgataagtcgtgtcttaccgggt |
| | tggactcaagacgatagttaccggataaggcgcagcggtcgggctgaacgggggg |
| | ttcgtgcacacagcccagcttggagcgaacgacctacaccgaactgagataccta |
| | cagcgtgagctatgagaaagcgccacgcttcccgaagggagaaaggcggacaggt |
| | atccggtaagcggcagggtcggaacaggagagcgcacgagggagcttccaggggg |
| | aaacgcctggtatctttatagtcctgtcgggtttcgccacctctgacttgagcgt |
| | cgatttttgtgatgctcgtcaggggggcggagcctatggaaaaacgccagcaacg |
| | cggcctttttacggttcctggccttttgctggccttttgctcacatgttctttcc |
| | tgcgttatcccctgattctgtggataaccgtattaccgcctttgagtgagctgat |
| | accgctcgccgcagccgaacgaccgagcgcagcgagtcagtgagcgaggaagcgg |
| | aagagcgcccaatacgcaaaccgcctctccccgcgcgttggccgattcattaatg |
| | cagctggcacgacaggtttcccgactggaaagcgggcagtgagcgcaacgcaatt |
| | aatgtgagttagctcactcattaggcaccccaggctttacactttatgcttccgg |
| | ctcgtatgttgtgtggaattgtgagcggataacaatttcacacaggaaacagcta |
| | tgaccatgattacgccaagctcgaaattaaccctcactaaagggaacaaaagctg |
| | gtacctcgcgcgacttggtttgccattctttagcgcgcgtcgcgtcacacagctt |
| | ggccacaatgtggtttttgtcaaacgaagattctatgacgtgtttaaagtttagg |
| | tcgagtaaagcgcaaatcttttttaaccctagaaagatagtctgcgtaaaattga |
| | cgcatgcattcttgaaatattgctctctctttctaaatagcgcgaatccgtcgct |
| | gtgcatttaggacatctcagtcgccgcttggagctcccgtgaggcgtgcttgtca |
| | atgcggtaagtgtcactgattttgaactataacgaccgcgtgagtcaaaatgacg |
| | catgattatcttttacgtgacttttaagatttaactcatacgataattatattgt |
| | tatttcatgttctacttacgtgataacttattatatatatattttcttgttatag |
| | atatcatcaactttgtatagaaaagttgctcgacattgattattgactagttatt |
| | aatagtaatcaattacggggtcattagttcatagcccatatatggagttccgcgt |
| | tacataacttacggtaaatggcccgcctggctgaccgcccaacgacccccgccca |
| | ttgacgtcaataatgacgtatgttcccatagtaacgccaatagggactttccatt |
| | gacgtcaatgggtggagtatttacggtaaactgcccacttggcagtacatcaagt |
| | gtatcatatgccaagtacgccccctattgacgtcaatgacggtaaatggcccgcc |
| | tggcattatgcccagtacatgaccttatgggactttcctacttggcagtacatct |
| | acgtattagtcatcgctattaccatggtcgaggtgagccccacgttctgcttcac |
| | tctccccatctcccccccctccccacccccaattttgtatttatttattttttaa |
| | ttattttgtgcagcgatgggggcggggggggggggggggcgcgcgccaggcgggg |
| | ggggcggggcgaggggcggggcggggcgaggcggagaggtgcggcggcagccaat |
| | cagagcggcgcgctccgaaagtttccttttatggcgaggcggcggcggcggcggc |
| | cctataaaaagcgaagcgcgcggggggggagtcgctgcgcgctgccttcgccccg |
| | tgccccgctccgccgccgcctcgcgccgcccgccccggctctgactgaccgcgtt |
| | actcccacaggtgagcggggggacggcccttctcctccgggctgtaattagcgct |
| | tggtttaatgacggcttgtttcttttctgtggctgcgtgaaagccttgaggggct |
| | ccgggagggccctttgtgcggggggagcggctcggggggtgcgtgcgtgtgtgtg |
| | tgcgtggggagcgccgcgtgcggctccgcgctgcccggcggctgtgagcgctgcg |
| | ggcgcggcgcggggctttgtgcgctccgcagtgtgcgcgaggggagcgcggccgg |
| | gggcggtgccccgcggtgcggggggggctgcgaggggaacaaaggctgcgtgcgg |
| | ggtgtgtgcgtgggggggtgagcagggggtgtgggcgcgtcggtcgggctgcaac |
| | cccccctgcacccccctccccgagttgctgagcacggcccggcttcgggtgcggg |
| | gctccgtacggggcgtggcgcggggctcgccgtgccgggcggggggggcggcagg |
| | tgggggtgccgggcggggcggggccgcctcgggccggggagggctcgggggaggg |
| | gcgcggcggcccccggagcgccggcggctgtcgaggcgcggcgagccgcagccat |
| | tgccttttatggtaatcgtgcgagagggcgcagggacttcctttgtcccaaatct |
| | gtgcggagccgaaatctgggaggcgccgccgcaccccctctagcgggcgcggggc |
| | gaagcggtgcggcgccggcaggaaggaaatgggcggggagggccttcgtgcgtcg |
| | ccgcgccgccgtccccttctccctctccagcctcggggctgtccgcggggggacg |
| | gctgccttcgggggggacggggcagggcggggttcggcttctggcgtgtgaccgg |
| | cggctctagagcctctgctaaccatgttcatgccttcttctttttcctacagctc |
| | ctgggcaacgtgctggttattgtgctgtctcatcattttggcaaagaattgcaag |
| | tttgtacaaaaaagcaggctGAATGCCGCCACCATGGCTaccgagtataaaccga |
| | cggtccgacttgcgactcgggatgacgttccacgcgccgtgaggactctggccgc |
| | cgccttcgctgactacccggccacacggcacacagttgaccccgacagacacata |
| | gagcgggtcacagagttgcaagaacttttcctgacgcgggttggActcgatattg |
| | gaaaagtttgggttgccgacgatggtgccgcagttgcggtgtggactactccaga |
| | aagtgtagaagctggagccgtattcgctgaaatagggccacggatggcagagctg |
| | tctggtagcaggctcgcagcgcagcagcagatggaaggCcttctcgctccgcatc |
| | ggcctaaggaaccggcgtggttcctggctacggtgggagtctcacctgaccatca |
| | aggaaagggattgggaagtgctgtcgttcttccaggcgtggaagcggccgaacga |
| | gcaggcgtcccagccttcttggaAacgagcgcacccaggaatcttccattctacg |
| | agcgactgggtttcactgtgacagcagacgtagaagtccctgaagggcctcggac |
| | ttggtgcatgaccagaaagcctggggcttgaAACGCCATgatccagacatgataa |
| | gatacattgatgagtttggacaaaccacaactagaatgcagtgaGGCGCGCCTGG |
| | ATACTTCTGCCTGCAGGGGAGTCGCtcttttgcaatgctctttgggaaattatat |
| | tagcctaattactaatttcctggccctcaaggtgacttgcttgaacttgccacct |
| | TCGCCACTAGGGGGCAGCAttggtttacacagggtgaacaaacgttcaattctaa |
| | actaatattctttggtgggaaagtgtgttcattttggtttgttttgaagaagtca |
| | cttcatacctttgaactggggttaagctggttaactcccaaaattccaccagttc |
| | caatatcctatggaaaaaccccaaaaccactCAAAccagatatactcgttgacat |
| | tgattattgactagttattaatagtaatcaattacggggtcattagttcatagcc |
| | catatatggagttccgcgttacataacttacggtaaatggcccgcctggctgacc |
| | gcccaacgacccccgcccattgacgtcaataatgacgtatgttcccatagtaacg |
| | ccaatagggactttccattgacgtcaatgggtggagtatttacggtaaactgccc |
| | acttggcagtacatcaagtgtatcatatgccaagtacgccccctattgacgtcaa |
| | tgacggtaaatggcccgcctggcattatgcccagtacatgaccttatgggacttt |
| | cctacttggcagtacatctacgtattagtcatcgctattaccatggtgatgcggt |
| | tttggcagtacatcaatgggcgtggatagcggtttgactcacggggatttccaag |
| | tctccaccccattgacgtcaatgggagtttgttttggcaccaaaatcaacgggac |
| | tttccaaaatgtcgtaacaactccgccccattgacgcaaatgggcggtaggcgtg |
| | tacggtgggaggtctatataagcagagctctctggctaactagagaacccactgc |
| | ttactggcttatcgaaattaatacgactcactatagggagaaccaagcGAATGCC |
| | GCCACCATGGCTgtgagcaagggcgaggagctgttcaccggggtggtgcccatcc |
| | tggtcgagctggacggcgacgtaaacggccacaagttcagcgtgtccggcgaggg |
| | cgagggcgatgccacctacggcaagctgaccctgaagttcatctgcaccaccggc |
| | aagctgcccgtgccctggcccaccctcgtgaccaccctgacctacggcgtgcagt |
| | gcttcagccgctaccccgaccacatgaagcagcacgacttcttcaagtccgccat |
| | gcccgaaggctacgtccaggagcgcaccatcttcttcaaggacgacggcaactac |
| | aagacccgcgccgaggtgaagttcgagggcgacaccctggtgaaccgcatcgagc |
| | tgaagggcatcgacttcaaggaggacggcaacatcctggggcacaagctggagta |
| | caactacaacagccacaacgtctatatcatggccgacaagcagaagaacggcatc |
| | aaggtgaacttcaagatccgccacaacatcgaggacggcagcgtgcagctcgccg |
| | accactaccagcagaacacccccatcggcgacggccccgtgctgctgcccgacaa |
| | ccactacctgagcacccagtccgccctgagcaaagaccccaacgagaagcgcgat |
| | cacatggtcctgctggagttcgtgaccgccgccgggatcactctcggcatggacg |
| | agctgtacaagtaaAACGCCATgaattcactcctcaggtgcaggctgcctatcag |
| | aaggtggtggctggtgtggccaatgccctggctcacaaataccactgagatcttt |
| | ttccctctgccaaaaattatggggacatcatgaagccccttgagcatctgacttc |
| | tggctaataaaggaaatttattttcattgcaatagtgtgttggaattttttgtgt |
| | ctctcactcggaaggacatatgggagggcaaatcatttaaaacatcagaatgagt |
| | atttggtttagagtttggcaacatatgcccatatgctggctgccatgaacaaagg |
| | ttggctataaagaggtcatcagtatatgaaacagccccctgctgtccattcctta |
| | ttccatagaaaagccttgacttgaggttagattttttttatattttgttttgtgt |
| | tatttttttctttaacatccctaaaattttccttacatgttttactagccagatt |
| | tttcctcctctcctgactactcccagtcatagctgtccctcttctcttatggaga |
| | tcCAGTGGCATCGCtcttttgcaatgctctttgggaaattatattagcctaatta |
| | ctaatttcctggccctcaaggtgacttgcttgaacttgccacctTCGCCACTAGG |
| | GGGCAGCAttggtttacacagggtgaacaaacgttcaattctaaactaatattct |
| | ttggtgggaaagtgtgttcattttggtttgttttgaagaagtcacttcatacctt |
| | tgaactggggttaagctggttaactcccaaaattccaccagttccaatatcctat |
| | ggaaaaaccccaaaaccactCAAAccagatatactcgttgacattgattattgac |
| | tagttattaatagtaatcaattacggggtcattagttcatagcccatatatggag |
| | ttccgcgttacataacttacggtaaatggcccgcctggctgaccgcccaacgacc |
| | cccgcccattgacgtcaataatgacgtatgttcccatagtaacgccaatagggac |
| | tttccattgacgtcaatgggtggagtatttacggtaaactgcccacttggcagta |
| | catcaagtgtatcatatgccaagtacgccccctattgacgtcaatgacggtaaat |
| | ggcccgcctggcattatgcccagtacatgaccttatgggactttcctacttggca |
| | gtacatctacgtattagtcatcgctattaccatggtgatgcggttttggcagtac |
| | atcaatgggcgtggatagcggtttgactcacggggatttccaagtctccacccca |
| | ttgacgtcaatgggagtttgttttggcaccaaaatcaacgggactttccaaaatg |
| | tcgtaacaactccgccccattgacgcaaatgggcggtaggcgtgtacggtgggag |
| | gtctatataagcagagctctctggctaactagagaacccactgcttactggctta |
| | tcgaaattaatacgactcactatagggagaaccaagcGAATGCCGCCACCATGGC |
| | TGTCAGTAAAGGGGAAgagcttataaaggaaaatatgcacatgaagctctacatg |
| | gagggcactgtagataaccaccatttcaaatgtacctctgaaggggagggcaagc |
| | catacgaaggtactcaaaccatgcgaataaaagtagttgaaggcgggcctcttcc |
| | ctttgcattcgacattctcgcaacctcttttctgtacggcagtaagactttcata |
| | aaccacactcaaggcattccagacttcttcaagcaatcattccccgaaggattca |
| | cctgggagcgagttactacttatgaggacggaggagtccttactgcaacccaaga |
| | cacctcactgcaagatgggtgcctgatttacaatgtaaagatcagaggggtgaat |
| | ttcacaagcaatgggccagttatgcaaaaaaagacccttggatgggaggccttca |
| | ccgagacactgtacccagccgatggtggactggagggcaggaatgacatggccct |
| | caagctcgtcggaggcagtcacttgattgccaacgccaaaaccacttaccgctct |
| | aagaaacctgctaaaaacctgaagatgcccggcgtctattatgtggactatcgac |
| | ttgagagaattaaggaggcaaacaacgagacttatgtcgaacagcatgaagttgc |
| | cgtggctaggtactgtgatttgcccagcaaattgggtcataaacttaacTGAAAC |
| | GCCATgaattcactcctcaggtgcaggctgcctatcagaaggtggtggctggtgt |
| | ggccaatgccctggctcacaaataccactgagatctttttccctctgccaaaaat |
| | tatggggacatcatgaagccccttgagcatctgacttctggctaataaaggaaat |
| | ttattttcattgcaatagtgtgttggaattttttgtgtctctcactcggaaggac |
| | atatgggagggcaaatcatttaaaacatcagaatgagtatttggtttagagtttg |
| | gcaacatatgcccatatgctggctgccatgaacaaaggttggctataaagaggtc |
| | atcagtatatgaaacagccccctgctgtccattccttattccatagaaaagcctt |
| | gacttgaggttagattttttttatattttgttttgtgttatttttttctttaaca |
| | tccctaaaattttccttacatgttttactagccagatttttcctcctctcctgac |
| | tactcccagtcatagctgtccctcttctcttatggagatcCAGTnnnnTCGCtct |
| | tttgcaatgctctttgggaaattatattagcctaattactaatttcctggccctc |
| | aaggtgacttgcttgaacttgccacctTCGCCACTAGGGGGCAGCAttggtttac |
| | acagggtgaacaaacgttcaattctaaactaatattctttggtgggaaagtgtgt |
| | tcattttggtttgttttgaagaagtcacttcatacctttgaactggggttaagct |
| | ggttaactcccaaaattccaccagttccaatatcctatggaaaaaccccaaaacc |
| | actCAAAccagatatactcgttgacattgattattgactagttattaatagtaat |
| | caattacggggtcattagttcatagcccatatatggagttccgcgttacataact |
| | tacggtaaatggcccgcctggctgaccgcccaacgacccccgcccattgacgtca |
| | ataatgacgtatgttcccatagtaacgccaatagggactttccattgacgtcaat |
| | gggtggagtatttacggtaaactgcccacttggcagtacatcaagtgtatcatat |
| | gccaagtacgccccctattgacgtcaatgacggtaaatggcccgcctggcattat |
| | gcccagtacatgaccttatgggactttcctacttggcagtacatctacgtattag |
| | tcatcgctattaccatggtgatgcggttttggcagtacatcaatgggcgtggata |
| | gcggtttgactcacggggatttccaagtctccaccccattgacgtcaatgggagt |
| | ttgttttggcaccaaaatcaacgggactttccaaaatgtcgtaacaactccgccc |
| | cattgacgcaaatgggcggtaggcgtgtacggtgggaggtctatataagcagagc |
| | tctctggctaactagagaacccactgcttactggcttatcgaaattaatacgact |
| | cactatagggagaaccaagcGAATGCCGCCACCATGGCTgctgaaggTtccgtcg |
| | ccaggcagcctgacctcttgacctgcgacgatgagccgatccatatccccggtgc |
| | catccaaccgcatggactgctgctcgccctcgccgccgacatgacgatcgttgcc |
| | ggcagcgacaaccttcccgaactcaccggactggcgatcggcgccctgatcggcc |
| | gctctgcggccgatgtGttcgactcggaAacgcacaaccgtctgacgatcgcctt |
| | ggccgagcccggggcggccgtcggagcaccgatcactgtcggcttcacgatgcga |
| | aaggacgcaggcttcatcggctcctggcatcgccatgatcagctcatcttcctGg |
| | agctGgagcctccccagcgggacgtcgccgagccgcaggcgttcttccgccgcac |
| | caacagcgccatccgccgcctgcaggccgccgaaaccttggaaagcgcctgcgcc |
| | gccgcggcgcaagaggtgcggaagattaccggcttcgatcgggtgatgatctatc |
| | gcttcgcctccgacttcagcggcgaagtgatcgcagaggatcggtgcgccgaggt |
| | cgagtcaaaactaggcctgcactatcctgcctcaaccgtgccggcgcaggcccgt |
| | cggctctataccatcaacccggtacggatcattcccgatatcaattatcggccgg |
| | tgccggtcaccccagacctcaatccggtcaccgggcggccgattgatcttagctt |
| | cgccatcctgcgcagcgtGtcgcccgtccatctggaGttcatgcgcaacataggc |
| | atgcacggcacgatgtcgatctcgattttgcgcggcgagcgactgtggggattga |
| | tcgtttgccatcaccgaacgccgtactacgtcgatctcgatggccgccaagcctg |
| | cgagctagtcgcccaggttctggcctggcagatcggcgtgatggaagagTAAAAC |
| | GCCATgaattcactcctcaggtgcaggctgcctatcagaaggtggtggctggtgt |
| | ggccaatgccctggctcacaaataccactgagatctttttccctctgccaaaaat |
| | tatggggacatcatgaagccccttgagcatctgacttctggctaataaaggaaat |
| | ttattttcattgcaatagtgtgttggaattttttgtgtctctcactcggaaggac |
| | atatgggagggcaaatcatttaaaacatcagaatgagtatttggtttagagtttg |
| | gcaacatatgcccatatgctggctgccatgaacaaaggttggctataaagaggtc |
| | atcagtatatgaaacagccccctgctgtccattccttattccatagaaaagcctt |
| | gacttgaggttagattttttttatattttgttttgtgttatttttttctttaaca |
| | tccctaaaattttccttacatgttttactagccagatttttcctcctctcctgac |
| | tactcccagtcatagctgtccctcttctcttatggagatcCAGTnnnnGCGGCCG |
| | CGCCTGGGCTTACttaattaacgagagcataatattgatatgtgccaaagttgtt |
| | tctgactgactaataagtataatttgtttctattatgtataggttaagctaatta |
| | cttattttataatacaacatgactgtttttaaagtacaaaataagtttatttttg |
| | taaaagagagaatgtttaaaagttttgttactttatagaagaaattttgagtttt |
| | tgtttttttttaataaataaataaacataaataaattgtttgttgaatttattat |
| | tagtatgtaagtgtaaatataataaaacttaatatctattcaaattaataaataa |
| | acctcgatatacagaccgataaaacacatgcgtcaattttacgcatgattatctt |
| | taacgtacgtcacaatatgattatctttctagggttaaataatagtttctaattt |
| | ttttattattcagcctgctgtcgtgaataccgagctccaattcgccctatagtga |
| | gtcgtattacaattcactggccgtcgttttacaacgtcgtgactgggaaaaccct |
| | ggcgttacccaacttaatcgccttgcagcacatccccctttcgccagctggcgta |
| | atagcgaagaggcccgcaccgatcgcccttcccaacagttgcgcagcctgaatgg |
| | cgaatgggacgcgccctgtagcggcgcattaagcgcggcgggtgtggtggttacg |
| | cgcagcgtgaccgctacacttgccagcgccctagcgcccgctcctttcgctttct |
| | tcccttcctttctcgccacgttcgccggctttccccgtcaagctctaaatcgggg |
| | gctccctttagggttccgatttagtgctttacggcacctcgaccccaaaaaactt |
| | gattagggtgatggttcacgtagtgggccatcgccctgatagacggtttttcgcc |
| | ctttgacgttggagtccacgttctttaatagtggactcttgttccaaactggaac |
| | aacactcaaccctatctcggtctattcttttgatttataagggattttgccgatt |
| | tcggcctattggttaaaaaatgagctgatttaacaaaaatttaacgcgaatttta |
| | acaaaatattaacgcttacaatttaggtggcacttttcggggaaatgtgcgcgga |
| | acccctatttgtttatttttctaaatacattcaaatatgtatccgctcatgagac |
| | aataaccctgataaatgcttcaataatattgaaaaaggaagagtatgagtattca |
| | acatttccgtgtcgcccttattcccttttttgcggcattttgccttcctgttttt |
| | gctcacccagaaacgctggtgaaagtaaaagatgctgaagatcagttgggtgcac |
| | gagtgggttacatcgaactggatctcaacagcggtaagatccttgagagttttcg |
| | ccccgaagaacgttttccaatgatgagcacttttaaagttctgctatgtggcgcg |
| | gtattatcccgtattgacgccgggcaagagcaactcggtcgccgcatacactatt |
| | ctcagaatgacttggttgagtactcaccagtcacagaaaagcatcttacggatgg |
| | catgacagtaagagaattatgcagtgctgccataaccatgagtgataacactgcg |
| | gccaacttacttctgacaacgatcggaggaccgaaggagctaaccgcttttttgc |
| | acaacatgggggatcatgtaactcgccttgatcgttgggaaccggagctgaatga |
| | agccataccaaacgacgagcgtgacaccacgatgcctgtagcaatggcaacaacg |
| | ttgcgcaaactattaactggcgaactacttactctagcttcccggcaacaattaa |
| | tagactggatggaggcggataaagttg |
| | (SEQ ID NO: 26) |
| pAJW_P33 | caggaccacttctgcgctcggcccttccggctggctggtttattgctgataaatc |
| | tggagccggtgagcgtgggtctcgcggtatcattgcagcactggggccagatggt |
| | aagccctcccgtatcgtagttatctacacgacggggagtcaggcaactatggatg |
| | aacgaaatagacagatcgctgagataggtgcctcactgattaagcattggtaact |
| | gtcagaccaagtttactcatatatactttagattgatttaaaacttcatttttaa |
| | tttaaaaggatctaggtgaagatcctttttgataatctcatgaccaaaatccctt |
| | aacgtgagttttcgttccactgagcgtcagaccccgtagaaaagatcaaaggatc |
| | ttcttgagatcctttttttctgcgcgtaatctgctgcttgcaaacaaaaaaacca |
| | ccgctaccagcggtggtttgtttgccggatcaagagctaccaactctttttccga |
| | aggtaactggcttcagcagagcgcagataccaaatactgttcttctagtgtagcc |
| | gtagttaggccaccacttcaagaactctgtagcaccgcctacatacctcgctctg |
| | ctaatcctgttaccagtggctgctgccagtggcgataagtcgtgtcttaccgggt |
| | tggactcaagacgatagttaccggataaggcgcagcggtcgggctgaacgggggg |
| | ttcgtgcacacagcccagcttggagcgaacgacctacaccgaactgagataccta |
| | cagcgtgagctatgagaaagcgccacgcttcccgaagggagaaaggcggacaggt |
| | atccggtaagcggcagggtcggaacaggagagcgcacgagggagcttccaggggg |
| | aaacgcctggtatctttatagtcctgtcgggtttcgccacctctgacttgagcgt |
| | cgatttttgtgatgctcgtcaggggggcggagcctatggaaaaacgccagcaacg |
| | cggcctttttacggttcctggccttttgctggccttttgctcacatgttctttcc |
| | tgcgttatcccctgattctgtggataaccgtattaccgcctttgagtgagctgat |
| | accgctcgccgcagccgaacgaccgagcgcagcgagtcagtgagcgaggaagcgg |
| | aagagcgcccaatacgcaaaccgcctctccccgcgcgttggccgattcattaatg |
| | cagctggcacgacaggtttcccgactggaaagcgggcagtgagcgcaacgcaatt |
| | aatgtgagttagctcactcattaggcaccccaggctttacactttatgcttccgg |
| | ctcgtatgttgtgtggaattgtgagcggataacaatttcacacaggaaacagcta |
| | tgaccatgattacgccaagctcgaaattaaccctcactaaagggaacaaaagctg |
| | gtacctcgcgcgacttggtttgccattctttagcgcgcgtcgcgtcacacagctt |
| | ggccacaatgtggtttttgtcaaacgaagattctatgacgtgtttaaagtttagg |
| | tcgagtaaagcgcaaatcttttttaaccctagaaagatagtctgcgtaaaattga |
| | cgcatgcattcttgaaatattgctctctctttctaaatagcgcgaatccgtcgct |
| | gtgcatttaggacatctcagtcgccgcttggagctcccgtgaggcgtgcttgtca |
| | atgcggtaagtgtcactgattttgaactataacgaccgcgtgagtcaaaatgacg |
| | catgattatcttttacgtgacttttaagatttaactcatacgataattatattgt |
| | tatttcatgttctacttacgtgataacttattatatatatattttcttgttatag |
| | atatcatcaactttgtatagaaaagttgctcgacattgattattgactagttatt |
| | aatagtaatcaattacggggtcattagttcatagcccatatatggagttccgcgt |
| | tacataacttacggtaaatggcccgcctggctgaccgcccaacgacccccgccca |
| | ttgacgtcaataatgacgtatgttcccatagtaacgccaatagggactttccatt |
| | gacgtcaatgggtggagtatttacggtaaactgcccacttggcagtacatcaagt |
| | gtatcatatgccaagtacgccccctattgacgtcaatgacggtaaatggcccgcc |
| | tggcattatgcccagtacatgaccttatgggactttcctacttggcagtacatct |
| | acgtattagtcatcgctattaccatggtcgaggtgagccccacgttctgcttcac |
| | tctccccatctcccccccctccccacccccaattttgtatttatttattttttaa |
| | ttattttgtgcagcgatgggggcggggggggggggggggcgcgcgccaggcgggg |
| | cggggcggggcgaggggggggcggggcgaggcggagaggtgcggcggcagccaat |
| | cagagcggcgcgctccgaaagtttccttttatggcgaggcggcggcggcggcggc |
| | cctataaaaagcgaagcgcgcggggggggagtcgctgcgcgctgccttcgccccg |
| | tgccccgctccgccgccgcctcgcgccgcccgccccggctctgactgaccgcgtt |
| | actcccacaggtgagcgggcgggacggcccttctcctccgggctgtaattagcgc |
| | ttggtttaatgacggcttgtttcttttctgtggctgcgtgaaagccttgaggggc |
| | tccgggagggccctttgtgcggggggagcggctcggggggtgcgtgcgtgtgtgt |
| | gtgcgtggggagcgccgcgtgcggctccgcgctgcccggcggctgtgagcgctgc |
| | gggcgcggcgcggggctttgtgcgctccgcagtgtgcgcgaggggagcgcggccg |
| | ggggcggtgccccgcggtgcggggggggctgcgaggggaacaaaggctgcgtgcg |
| | gggtgtgtgcgtgggggggtgagcagggggtgtgggcgcgtcggtcgggctgcaa |
| | ccccccctgcacccccctccccgagttgctgagcacggcccggcttcgggtgcgg |
| | ggctccgtacggggcgtggcgcggggctcgccgtgccgggggggggtggcggcag |
| | gtgggggtgccgggcggggcggggccgcctcgggccggggagggctcgggggggg |
| | ggggcggcccccggagcgccggcggctgtcgaggcgcggcgagccgcagccattg |
| | ccttttatggtaatcgtgcgagagggcgcagggacttcctttgtcccaaatctgt |
| | gcggagccgaaatctgggaggcgccgccgcaccccctctagcgggcgcggggcga |
| | agcggtgcggcgccggcaggaaggaaatgggcggggagggccttcgtgcgtcgcc |
| | gcgccgccgtccccttctccctctccagcctcggggctgtccgcggggggacggc |
| | tgccttcgggggggacggggcagggcggggttcggcttctggcgtgtgaccggcg |
| | gctctagagcctctgctaaccatgttcatgccttcttctttttcctacagctcct |
| | gggcaacgtgctggttattgtgctgtctcatcattttggcaaagaattgcaagtt |
| | tgtacaaaaaagcaggctGAATGCCGCCACCATGGCTaccgagtataaaccgacg |
| | gtccgacttgcgactcgggatgacgttccacgcgccgtgaggactctggccgccg |
| | ccttcgctgactacccggccacacggcacacagttgaccccgacagacacataga |
| | gcgggtcacagagttgcaagaacttttcctgacgcgggttggActcgatattgga |
| | aaagtttgggttgccgacgatggtgccgcagttgcggtgtggactactccagaaa |
| | gtgtagaagctggagccgtattcgctgaaatagggccacggatggcagagctgtc |
| | tggtagcaggctcgcagcgcagcagcagatggaaggCcttctcgctccgcatcgg |
| | cctaaggaaccggcgtggttcctggctacggtgggagtctcacctgaccatcaag |
| | gaaagggattgggaagtgctgtcgttcttccaggcgtggaagcggccgaacgagc |
| | aggcgtcccagccttcttggaAacgagcgcacccaggaatcttccattctacgag |
| | cgactgggtttcactgtgacagcagacgtagaagtccctgaagggcctcggactt |
| | ggtgcatgaccagaaagcctggggcttgaAACGCCATgatccagacatgataaga |
| | tacattgatgagtttggacaaaccacaactagaatgcagtgaGGCGCGCCTGGAT |
| | ACTTCTGCCTGCAGGGGAGTCGCtcttttgcaatgctctttgggaaattatatta |
| | gcctaattactaatttcctggccctcaaggtgacttgcttgaacttgccacctTC |
| | GCCACTAGGGGGCAGCAttggtttacacagggtgaacaaacgttcaattctaaac |
| | taatattctttggtgggaaagtgtgttcattttggtttgttttgaagaagtcact |
| | tcatacctttgaactggggttaagctggttaactcccaaaattccaccagttcca |
| | atatcctatggaaaaaccccaaaaccactCAAAccagatatactcgttgacattg |
| | attattgactagttattaatagtaatcaattacggggtcattagttcatagccca |
| | tatatggagttccgcgttacataacttacggtaaatggcccgcctggctgaccgc |
| | ccaacgacccccgcccattgacgtcaataatgacgtatgttcccatagtaacgcc |
| | aatagggactttccattgacgtcaatgggtggagtatttacggtaaactgcccac |
| | ttggcagtacatcaagtgtatcatatgccaagtacgccccctattgacgtcaatg |
| | acggtaaatggcccgcctggcattatgcccagtacatgaccttatgggactttcc |
| | tacttggcagtacatctacgtattagtcatcgctattaccatggtgatgcggttt |
| | tggcagtacatcaatgggcgtggatagcggtttgactcacggggatttccaagtc |
| | tccaccccattgacgtcaatgggagtttgttttggcaccaaaatcaacgggactt |
| | tccaaaatgtcgtaacaactccgccccattgacgcaaatgggcggtaggcgtgta |
| | cggtgggaggtctatataagcagagctctctggctaactagagaacccactgctt |
| | actggcttatcgaaattaatacgactcactatagggagaaccaagcGAATGCCGC |
| | CACCATGGCTgtgagcaagggcgaggagctgttcaccggggtggtgcccatcctg |
| | gtcgagctggacggcgacgtaaacggccacaagttcagcgtgtccggcgagggcg |
| | agggcgatgccacctacggcaagctgaccctgaagttcatctgcaccaccggcaa |
| | gctgcccgtgccctggcccaccctcgtgaccaccctgacctacggcgtgcagtgc |
| | ttcagccgctaccccgaccacatgaagcagcacgacttcttcaagtccgccatgc |
| | ccgaaggctacgtccaggagcgcaccatcttcttcaaggacgacggcaactacaa |
| | gacccgcgccgaggtgaagttcgagggcgacaccctggtgaaccgcatcgagctg |
| | aagggcatcgacttcaaggaggacggcaacatcctggggcacaagctggagtaca |
| | actacaacagccacaacgtctatatcatggccgacaagcagaagaacggcatcaa |
| | ggtgaacttcaagatccgccacaacatcgaggacggcagcgtgcagctcgccgac |
| | cactaccagcagaacacccccatcggcgacggccccgtgctgctgcccgacaacc |
| | actacctgagcacccagtccgccctgagcaaagaccccaacgagaagcgcgatca |
| | catggtcctgctggagttcgtgaccgccgccgggatcactctcggcatggacgag |
| | ctgtacaagtaaAACGCCATgaattcactcctcaggtgcaggctgcctatcagaa |
| | ggtggtggctggtgtggccaatgccctggctcacaaataccactgagatcttttt |
| | ccctctgccaaaaattatggggacatcatgaagccccttgagcatctgacttctg |
| | gctaataaaggaaatttattttcattgcaatagtgtgttggaattttttgtgtct |
| | ctcactcggaaggacatatgggagggcaaatcatttaaaacatcagaatgagtat |
| | ttggtttagagtttggcaacatatgcccatatgctggctgccatgaacaaaggtt |
| | ggctataaagaggtcatcagtatatgaaacagccccctgctgtccattccttatt |
| | ccatagaaaagccttgacttgaggttagattttttttatattttgttttgtgtta |
| | tttttttctttaacatccctaaaattttccttacatgttttactagccagatttt |
| | tcctcctctcctgactactcccagtcatagctgtccctcttctcttatggagatc |
| | CAGTGGCATCGCtcttttgcaatgctctttgggaaattatattagcctaattact |
| | aatttcctggccctcaaggtgacttgcttgaacttgccacctTCGCCACTAGGGG |
| | GCAGCAttggtttacacagggtgaacaaacgttcaattctaaactaatattcttt |
| | ggtgggaaagtgtgttcattttggtttgttttgaagaagtcacttcatacctttg |
| | aactggggttaagctggttaactcccaaaattccaccagttccaatatcctatgg |
| | aaaaaccccaaaaccactCAAAccagatatactcgttgacattgattattgacta |
| | gttattaatagtaatcaattacggggtcattagttcatagcccatatatggagtt |
| | ccgcgttacataacttacggtaaatggcccgcctggctgaccgcccaacgacccc |
| | cgcccattgacgtcaataatgacgtatgttcccatagtaacgccaatagggactt |
| | tccattgacgtcaatgggtggagtatttacggtaaactgcccacttggcagtaca |
| | tcaagtgtatcatatgccaagtacgccccctattgacgtcaatgacggtaaatgg |
| | cccgcctggcattatgcccagtacatgaccttatgggactttcctacttggcagt |
| | acatctacgtattagtcatcgctattaccatggtgatgcggttttggcagtacat |
| | caatgggcgtggatagcggtttgactcacggggatttccaagtctccaccccatt |
| | gacgtcaatgggagtttgttttggcaccaaaatcaacgggactttccaaaatgtc |
| | gtaacaactccgccccattgacgcaaatgggcggtaggcgtgtacggtgggaggt |
| | ctatataagcagagctctctggctaactagagaacccactgcttactggcttatc |
| | gaaattaatacgactcactatagggagaaccaagcGAATGCCGCCACCATGGCTG |
| | TCAGTAAAGGGGAAgagcttataaaggaaaatatgcacatgaagctctacatgga |
| | gggcactgtagataaccaccatttcaaatgtacctctgaaggggagggcaagcca |
| | tacgaaggtactcaaaccatgcgaataaaagtagttgaaggcgggcctcttccct |
| | ttgcattcgacattctcgcaacctcttttctgtacggcagtaagactttcataaa |
| | ccacactcaaggcattccagacttcttcaagcaatcattccccgaaggattcacc |
| | tgggagcgagttactacttatgaggacggaggagtccttactgcaacccaagaca |
| | cctcactgcaagatgggtgcctgatttacaatgtaaagatcagaggggtgaattt |
| | cacaagcaatgggccagttatgcaaaaaaagacccttggatgggaggccttcacc |
| | gagacactgtacccagccgatggtggactggagggcaggaatgacatggccctca |
| | agctcgtcggaggcagtcacttgattgccaacgccaaaaccacttaccgctctaa |
| | gaaacctgctaaaaacctgaagatgcccggcgtctattatgtggactatcgactt |
| | gagagaattaaggaggcaaacaacgagacttatgtcgaacagcatgaagttgccg |
| | tggctaggtactgtgatttgcccagcaaattgggtcataaacttaacTGAAACGC |
| | CATgaattcactcctcaggtgcaggctgcctatcagaaggtggtggctggtgtgg |
| | ccaatgccctggctcacaaataccactgagatctttttccctctgccaaaaatta |
| | tggggacatcatgaagccccttgagcatctgacttctggctaataaaggaaattt |
| | attttcattgcaatagtgtgttggaattttttgtgtctctcactcggaaggacat |
| | atgggagggcaaatcatttaaaacatcagaatgagtatttggtttagagtttggc |
| | aacatatgcccatatgctggctgccatgaacaaaggttggctataaagaggtcat |
| | cagtatatgaaacagccccctgctgtccattccttattccatagaaaagccttga |
| | cttgaggttagattttttttatattttgttttgtgttatttttttctttaacatc |
| | cctaaaattttccttacatgttttactagccagatttttcctcctctcctgacta |
| | ctcccagtcatagctgtccctcttctcttatggagatcCAGTnnnnTCGCtcttt |
| | tgcaatgctctttgggaaattatattagcctaattactaatttcctggccctcaa |
| | ggtgacttgcttgaacttgccacctTCGCCACTAGGGGGCAGCAttggtttacac |
| | agggtgaacaaacgttcaattctaaactaatattctttggtgggaaagtgtgttc |
| | attttggtttgttttgaagaagtcacttcatacctttgaactggggttaagctgg |
| | ttaactcccaaaattccaccagttccaatatcctatggaaaaaccccaaaaccac |
| | tCAAAccagatatactcgttgacattgattattgactagttattaatagtaatca |
| | attacggggtcattagttcatagcccatatatggagttccgcgttacataactta |
| | cggtaaatggcccgcctggctgaccgcccaacgacccccgcccattgacgtcaat |
| | aatgacgtatgttcccatagtaacgccaatagggactttccattgacgtcaatgg |
| | gtggagtatttacggtaaactgcccacttggcagtacatcaagtgtatcatatgc |
| | caagtacgccccctattgacgtcaatgacggtaaatggcccgcctggcattatgc |
| | ccagtacatgaccttatgggactttcctacttggcagtacatctacgtattagtc |
| | atcgctattaccatggtgatgcggttttggcagtacatcaatgggcgtggatagc |
| | ggtttgactcacggggatttccaagtctccaccccattgacgtcaatgggagttt |
| | gttttggcaccaaaatcaacgggactttccaaaatgtcgtaacaactccgcccca |
| | ttgacgcaaatgggcggtaggcgtgtacggtgggaggtctatataagcagagctc |
| | tctggctaactagagaacccactgcttactggcttatcgaaattaatacgactca |
| | ctatagggagaaccaagcGAATGCCGCCACCATGGCTgctgaaggTtccgtcgcc |
| | aggcagcctgacctcttgacctgcgacgatgagccgatccatatccccggtgcca |
| | tccaaccgcatggactgctgctcgccctcgccgccgacatgacgatcgttgccgg |
| | cagcgacaaccttcccgaactcaccggactggcgatcggcgccctgatcggccgc |
| | tctgcggccgatgtGttcgactcggaAacgcacaaccgtctgacgatcgccttgg |
| | ccgagcccggggcggccgtcggagcaccgatcactgtcggcttcacgatgcgaaa |
| | ggacgcaggcttcatcggctcctggcatcgccatgatcagctcatcttcctGgag |
| | ctGgagcctccccaggggacgtcgccgagccgcaggcgttcttccgccgcaccaa |
| | cagcgccatccgccgcctgcaggccgccgaaaccttggaaagcgcctgcgccgcc |
| | gcggcgcaagaggtgcggaagattaccggcttcgatcgggtgatgatctatcgct |
| | tcgcctccgacttcagcggcgaagtgatcgcagaggatcggtgcgccgaggtcga |
| | gtcaaaactaggcctgcactatcctgcctcaaccgtgccggcgcaggcccgtcgg |
| | ctctataccatcaacccggtacggatcattcccgatatcaattatcggccggtgc |
| | cggtcaccccagacctcaatccggtcaccgggcggccgattgatcttagcttcgc |
| | catcctgcgcagcgtGtcgcccgtccatctggaGttcatgcgcaacataggcatg |
| | cacggcacgatgtcgatctcgattttgcgcggcgagcgactgtggggattgatcg |
| | tttgccatcaccgaacgccgtactacgtcgatctcgatggccgccaagcctgcga |
| | gctagtcgcccaggttctggcctggcagatcggcgtgatggaagagTAAAACGCC |
| | ATgaattcactcctcaggtgcaggctgcctatcagaaggtggtggctggtgtggc |
| | caatgccctggctcacaaataccactgagatctttttccctctgccaaaaattat |
| | ggggacatcatgaagccccttgagcatctgacttctggctaataaaggaaattta |
| | ttttcattgcaatagtgtgttggaattttttgtgtctctcactcggaaggacata |
| | tgggagggcaaatcatttaaaacatcagaatgagtatttggtttagagtttggca |
| | acatatgcccatatgctggctgccatgaacaaaggttggctataaagaggtcatc |
| | agtatatgaaacagccccctgctgtccattccttattccatagaaaagccttgac |
| | ttgaggttagattttttttatattttgttttgtgttatttttttctttaacatcc |
| | ctaaaattttccttacatgttttactagccagatttttcctcctctcctgactac |
| | tcccagtcatagctgtccctcttctcttatggagatcCAGTnnnnTCGCtctttt |
| | gcaatgctctttgggaaattatattagcctaattactaatttcctggccctcaag |
| | gtgacttgcttgaacttgccacctTCGCCACTAGGGGGCAGCAttggtttacaca |
| | gggtgaacaaacgttcaattctaaactaatattctttggtgggaaagtgtgttca |
| | ttttggtttgttttgaagaagtcacttcatacctttgaactggggttaagctggt |
| | taactcccaaaattccaccagttccaatatcctatggaaaaaccccaaaaccact |
| | CAAAccagatatactcgttgacattgattattgactagttattaatagtaatcaa |
| | ttacggggtcattagttcatagcccatatatggagttccgcgttacataacttac |
| | ggtaaatggcccgcctggctgaccgcccaacgacccccgcccattgacgtcaata |
| | atgacgtatgttcccatagtaacgccaatagggactttccattgacgtcaatggg |
| | tggagtatttacggtaaactgcccacttggcagtacatcaagtgtatcatatgcc |
| | aagtacgccccctattgacgtcaatgacggtaaatggcccgcctggcattatgcc |
| | cagtacatgaccttatgggactttcctacttggcagtacatctacgtattagtca |
| | tcgctattaccatggtgatgcggttttggcagtacatcaatgggcgtggatagcg |
| | gtttgactcacggggatttccaagtctccaccccattgacgtcaatgggagtttg |
| | ttttggcaccaaaatcaacgggactttccaaaatgtcgtaacaactccgccccat |
| | tgacgcaaatgggcggtaggcgtgtacggtgggaggtctatataagcagagctct |
| | ctggctaactagagaacccactgcttactggcttatcgaaattaatacgactcac |
| | tatagggagaaccaagcGAATGCCGCCACCATGGCTggcaatagcgttagcaaag |
| | gtgaggaagaTaacatggcgattataaaggaatttatgcggttcaaggtgcacat |
| | ggagggttcagttaatggacacgagttcgaaatcgaaggtgagggggaaggtaga |
| | ccttacgaaggaactcagaccgccaagctgaaggtgaccaagggggccccctgcc |
| | cttcgcctgggacatcctgtcccctcagttcatgtacggctccaaggcctacgtg |
| | aagcaccccgccgacatccccgactacttgaagctgtccttccccgagggcttca |
| | agtgggagcgcgtgatgaacttcgaggacggcggcgtggtgaccgtgacccagga |
| | ctcctccttgcaggacggcgagttcatctacaaggtgaagctgcgcggcaccaac |
| | ttcccctccgacggccccgtaatgcagaagaaaaccatgggctggcaggcctcct |
| | ccgagcggatgtaccccgaggacggcgccctgaagggcgagatcaagcagaggct |
| | gaagctgaaggacggcggccactacgacgctgaggtcaagaccacctacaaggcc |
| | aagaagcccgtgcagctgcccggcgcctacaacgtcaacatcaagttggacatca |
| | cctcccacaacgaggactacaccatcgtggaacagtacgaacgcgccgagggccg |
| | ccactccaccggcggcatggacgagctgtataagTAAAACGCCATgaattcactc |
| | ctcaggtgcaggctgcctatcagaaggtggtggctggtgtggccaatgccctggc |
| | tcacaaataccactgagatctttttccctctgccaaaaattatggggacatcatg |
| | aagccccttgagcatctgacttctggctaataaaggaaatttattttcattgcaa |
| | tagtgtgttggaattttttgtgtctctcactcggaaggacatatgggagggcaaa |
| | tcatttaaaacatcagaatgagtatttggtttagagtttggcaacatatgcccat |
| | atgctggctgccatgaacaaaggttggctataaagaggtcatcagtatatgaaac |
| | agccccctgctgtccattccttattccatagaaaagccttgacttgaggttagat |
| | tttttttatattttgttttgtgttatttttttctttaacatccctaaaattttcc |
| | ttacatgttttactagccagatttttcctcctctcctgactactcccagtcatag |
| | ctgtccctcttctcttatggagatcCAGTnnnnGCGGCCGCGCCTGGGCTTACtt |
| | aattaacgagagcataatattgatatgtgccaaagttgtttctgactgactaata |
| | agtataatttgtttctattatgtataggttaagctaattacttattttataatac |
| | aacatgactgtttttaaagtacaaaataagtttatttttgtaaaagagagaatgt |
| | ttaaaagttttgttactttatagaagaaattttgagtttttgtttttttttaata |
| | aataaataaacataaataaattgtttgttgaatttattattagtatgtaagtgta |
| | aatataataaaacttaatatctattcaaattaataaataaacctcgatatacaga |
| | ccgataaaacacatgcgtcaattttacgcatgattatctttaacgtacgtcacaa |
| | tatgattatctttctagggttaaataatagtttctaatttttttattattcagcc |
| | tgctgtcgtgaataccgagctccaattcgccctatagtgagtcgtattacaattc |
| | actggccgtcgttttacaacgtcgtgactgggaaaaccctggcgttacccaactt |
| | aatcgccttgcagcacatccccctttcgccagctggcgtaatagcgaagaggccc |
| | gcaccgatcgcccttcccaacagttgcgcagcctgaatggcgaatgggacgcgcc |
| | ctgtagcggcgcattaagcgcggcgggtgtggtggttacgcgcagcgtgaccgct |
| | acacttgccagcgccctagcgcccgctcctttcgctttcttcccttcctttctcg |
| | ccacgttcgccggctttccccgtcaagctctaaatcgggggctccctttagggtt |
| | ccgatttagtgctttacggcacctcgaccccaaaaaacttgattagggtgatggt |
| | tcacgtagtgggccatcgccctgatagacggtttttcgccctttgacgttggagt |
| | ccacgttctttaatagtggactcttgttccaaactggaacaacactcaaccctat |
| | ctcggtctattcttttgatttataagggattttgccgatttcggcctattggtta |
| | aaaaatgagctgatttaacaaaaatttaacgcgaattttaacaaaatattaacgc |
| | ttacaatttaggtggcacttttcggggaaatgtgcgcggaacccctatttgttta |
| | tttttctaaatacattcaaatatgtatccgctcatgagacaataaccctgataaa |
| | tgcttcaataatattgaaaaaggaagagtatgagtattcaacatttccgtgtcgc |
| | ccttattcccttttttgcggcattttgccttcctgtttttgctcacccagaaacg |
| | ctggtgaaagtaaaagatgctgaagatcagttgggtgcacgagtgggttacatcg |
| | aactggatctcaacagcggtaagatccttgagagttttcgccccgaagaacgttt |
| | tccaatgatgagcacttttaaagttctgctatgtggcgcggtattatcccgtatt |
| | gacgccgggcaagagcaactcggtcgccgcatacactattctcagaatgacttgg |
| | ttgagtactcaccagtcacagaaaagcatcttacggatggcatgacagtaagaga |
| | attatgcagtgctgccataaccatgagtgataacactgcggccaacttacttctg |
| | acaacgatcggaggaccgaaggagctaaccgcttttttgcacaacatgggggatc |
| | atgtaactcgccttgatcgttgggaaccggagctgaatgaagccataccaaacga |
| | cgagcgtgacaccacgatgcctgtagcaatggcaacaacgttgcgcaaactatta |
| | actggcgaactacttactctagcttcccggcaacaattaatagactggatggagg |
| | cggataaagttg |
| | (SEQ ID NO: 27) |
| pAJW_P48 | caggaccacttctgcgctcggcccttccggctggctggtttattgctgataaatc |
| | tggagccggtgagcgtgggtctcgcggtatcattgcagcactggggccagatggt |
| | aagccctcccgtatcgtagttatctacacgacggggagtcaggcaactatggatg |
| | aacgaaatagacagatcgctgagataggtgcctcactgattaagcattggtaact |
| | gtcagaccaagtttactcatatatactttagattgatttaaaacttcatttttaa |
| | tttaaaaggatctaggtgaagatcctttttgataatctcatgaccaaaatccctt |
| | aacgtgagttttcgttccactgagcgtcagaccccgtagaaaagatcaaaggatc |
| | ttcttgagatcctttttttctgcgcgtaatctgctgcttgcaaacaaaaaaacca |
| | ccgctaccagcggtggtttgtttgccggatcaagagctaccaactctttttccga |
| | aggtaactggcttcagcagagcgcagataccaaatactgttcttctagtgtagcc |
| | gtagttaggccaccacttcaagaactctgtagcaccgcctacatacctcgctctg |
| | ctaatcctgttaccagtggctgctgccagtggcgataagtcgtgtcttaccgggt |
| | tggactcaagacgatagttaccggataaggcgcagcggtcgggctgaacgggggg |
| | ttcgtgcacacagcccagcttggagcgaacgacctacaccgaactgagataccta |
| | cagcgtgagctatgagaaagcgccacgcttcccgaagggagaaaggcggacaggt |
| | atccggtaagcggcagggtcggaacaggagagcgcacgagggagcttccaggggg |
| | aaacgcctggtatctttatagtcctgtcgggtttcgccacctctgacttgagcgt |
| | cgatttttgtgatgctcgtcaggggggcggagcctatggaaaaacgccagcaacg |
| | cggcctttttacggttcctggccttttgctggccttttgctcacatgttctttcc |
| | tgcgttatcccctgattctgtggataaccgtattaccgcctttgagtgagctgat |
| | accgctcgccgcagccgaacgaccgagcgcagcgagtcagtgagcgaggaagcgg |
| | aagagcgcccaatacgcaaaccgcctctccccgcgcgttggccgattcattaatg |
| | cagctggcacgacaggtttcccgactggaaagcgggcagtgagcgcaacgcaatt |
| | aatgtgagttagctcactcattaggcaccccaggctttacactttatgcttccgg |
| | ctcgtatgttgtgtggaattgtgagcggataacaatttcacacaggaaacagcta |
| | tgaccatgattacgccaagctcgaaattaaccctcactaaagggaacaaaagctg |
| | gtacctcgcgcgacttggtttgccattctttagcgcgcgtcgcgtcacacagctt |
| | ggccacaatgtggtttttgtcaaacgaagattctatgacgtgtttaaagtttagg |
| | tcgagtaaagcgcaaatcttttttaaccctagaaagatagtctgcgtaaaattga |
| | cgcatgcattcttgaaatattgctctctctttctaaatagcgcgaatccgtcgct |
| | gtgcatttaggacatctcagtcgccgcttggagctcccgtgaggcgtgcttgtca |
| | atgcggtaagtgtcactgattttgaactataacgaccgcgtgagtcaaaatgacg |
| | catgattatcttttacgtgacttttaagatttaactcatacgataattatattgt |
| | tatttcatgttctacttacgtgataacttattatatatatattttcttgttatag |
| | atatcatcaactttgtatagaaaagttgctcgacattgattattgactagttatt |
| | aatagtaatcaattacggggtcattagttcatagcccatatatggagttccgcgt |
| | tacataacttacggtaaatggcccgcctggctgaccgcccaacgacccccgccca |
| | ttgacgtcaataatgacgtatgttcccatagtaacgccaatagggactttccatt |
| | gacgtcaatgggtggagtatttacggtaaactgcccacttggcagtacatcaagt |
| | gtatcatatgccaagtacgccccctattgacgtcaatgacggtaaatggcccgcc |
| | tggcattatgcccagtacatgaccttatgggactttcctacttggcagtacatct |
| | acgtattagtcatcgctattaccatggtcgaggtgagccccacgttctgcttcac |
| | tctccccatctcccccccctccccacccccaattttgtatttatttattttttaa |
| | ttattttgtgcagcgatgggggcggggggggggggggggcgcgcgccaggcgggg |
| | cggggcggggcgaggggcggggggggcgaggcggagaggtgcggcggcagccaat |
| | cagagcggcgcgctccgaaagtttccttttatggcgaggggcggcggcggcggcc |
| | ctataaaaagcgaagcgcgcggggggggagtcgctgcgcgctgccttcgccccgt |
| | gccccgctccgccgccgcctcgcgccgcccgccccggctctgactgaccgcgtta |
| | ctcccacaggtgagcggggggacggcccttctcctccgggctgtaattagcgctt |
| | ggtttaatgacggcttgtttcttttctgtggctgcgtgaaagccttgaggggctc |
| | cgggagggccctttgtgcggggggagcggctcggggggtgcgtgcgtgtgtgtgt |
| | gcgtggggagcgccgcgtgcggctccgcgctgcccggcggctgtgagcgctgcgg |
| | gcgcggcgcggggctttgtgcgctccgcagtgtgcgcgaggggagcgcggccggg |
| | ggcggtgccccgcggtgcggggggggctgcgaggggaacaaaggctgcgtgcggg |
| | gtgtgtgcgtgggggggtgagcagggggtgtgggcgcgtcggtcgggctgcaacc |
| | ccccctgcacccccctccccgagttgctgagcacggcccggcttcgggtgcgggg |
| | ctccgtacggggcgtggcgcggggctcgccgtgccgggggggggtggcggcaggt |
| | gggggtgccgggcggggcggggccgcctcgggccggggagggctcgggggagggg |
| | cgcggcggcccccggagcgccggcggctgtcgaggcgcggcgagccgcagccatt |
| | gccttttatggtaatcgtgcgagagggcgcagggacttcctttgtcccaaatctg |
| | tgcggagccgaaatctgggaggcgccgccgcaccccctctagcgggcgcggggcg |
| | aagcggtgcggcgccggcaggaaggaaatgggcggggagggccttcgtgcgtcgc |
| | cgcgccgccgtccccttctccctctccagcctcggggctgtccgcggggggacgg |
| | ctgccttcgggggggacggggcagggcggggttcggcttctggcgtgtgaccggc |
| | ggctctagagcctctgctaaccatgttcatgccttcttctttttcctacagctcc |
| | tgggcaacgtgctggttattgtgctgtctcatcattttggcaaagaattgcaagt |
| | ttgtacaaaaaagcaggctGAATGCCGCCACCATGGCTaccgagtataaaccgac |
| | ggtccgacttgcgactcgggatgacgttccacgcgccgtgaggactctggccgcc |
| | gccttcgctgactacccggccacacggcacacagttgaccccgacagacacatag |
| | agcgggtcacagagttgcaagaacttttcctgacgcgggttggActcgatattgg |
| | aaaagtttgggttgccgacgatggtgccgcagttgcggtgtggactactccagaa |
| | agtgtagaagctggagccgtattcgctgaaatagggccacggatggcagagctgt |
| | ctggtagcaggctcgcagcgcagcagcagatggaaggCcttctcgctccgcatcg |
| | gcctaaggaaccggcgtggttcctggctacggtgggagtctcacctgaccatcaa |
| | ggaaagggattgggaagtgctgtcgttcttccaggcgtggaagcggccgaacgag |
| | caggcgtcccagccttcttggaAacgagcgcacccaggaatcttccattctacga |
| | gcgactgggtttcactgtgacagcagacgtagaagtccctgaagggcctcggact |
| | tggtgcatgaccagaaagcctggggcttgaAACGCCATgatccagacatgataag |
| | atacattgatgagtttggacaaaccacaactagaatgcagtgaGGCGCGCCTGGA |
| | TACTTCTGCCTGCAGGTTCATTAACGACGCGGCCGCGCCTGGGCTTACttaatta |
| | acgagagcataatattgatatgtgccaaagttgtttctgactgactaataagtat |
| | aatttgtttctattatgtataggttaagctaattacttattttataatacaacat |
| | gactgtttttaaagtacaaaataagtttatttttgtaaaagagagaatgtttaaa |
| | agttttgttactttatagaagaaattttgagtttttgtttttttttaataaataa |
| | ataaacataaataaattgtttgttgaatttattattagtatgtaagtgtaaatat |
| | aataaaacttaatatctattcaaattaataaataaacctcgatatacagaccgat |
| | aaaacacatgcgtcaattttacgcatgattatctttaacgtacgtcacaatatga |
| | ttatctttctagggttaaataatagtttctaatttttttattattcagcctgctg |
| | tcgtgaataccgagctccaattcgccctatagtgagtcgtattacaattcactgg |
| | ccgtcgttttacaacgtcgtgactgggaaaaccctggcgttacccaacttaatcg |
| | ccttgcagcacatccccctttcgccagctggcgtaatagcgaagaggcccgcacc |
| | gatcgcccttcccaacagttgcgcagcctgaatggcgaatgggacgcgccctgta |
| | gcggcgcattaagcgcggcgggtgtggtggttacgcgcagcgtgaccgctacact |
| | tgccagcgccctagcgcccgctcctttcgctttcttcccttcctttctcgccacg |
| | ttcgccggctttccccgtcaagctctaaatcgggggctccctttagggttccgat |
| | ttagtgctttacggcacctcgaccccaaaaaacttgattagggtgatggttcacg |
| | tagtgggccatcgccctgatagacggtttttcgccctttgacgttggagtccacg |
| | ttctttaatagtggactcttgttccaaactggaacaacactcaaccctatctcgg |
| | tctattcttttgatttataagggattttgccgatttcggcctattggttaaaaaa |
| | tgagctgatttaacaaaaatttaacgcgaattttaacaaaatattaacgcttaca |
| | atttaggtggcacttttcggggaaatgtgcgcggaacccctatttgtttattttt |
| | ctaaatacattcaaatatgtatccgctcatgagacaataaccctgataaatgctt |
| | caataatattgaaaaaggaagagtatgagtattcaacatttccgtgtcgccctta |
| | ttcccttttttgcggcattttgccttcctgtttttgctcacccagaaacgctggt |
| | gaaagtaaaagatgctgaagatcagttgggtgcacgagtgggttacatcgaactg |
| | gatctcaacagcggtaagatccttgagagttttcgccccgaagaacgttttccaa |
| | tgatgagcacttttaaagttctgctatgtggcgcggtattatcccgtattgacgc |
| | cgggcaagagcaactcggtcgccgcatacactattctcagaatgacttggttgag |
| | tactcaccagtcacagaaaagcatcttacggatggcatgacagtaagagaattat |
| | gcagtgctgccataaccatgagtgataacactgcggccaacttacttctgacaac |
| | gatcggaggaccgaaggagctaaccgcttttttgcacaacatgggggatcatgta |
| | actcgccttgatcgttgggaaccggagctgaatgaagccataccaaacgacgagc |
| | gtgacaccacgatgcctgtagcaatggcaacaacgttgcgcaaactattaactgg |
| | cgaactacttactctagcttcccggcaacaattaatagactggatggaggcggat |
| | aaagttg |
| | (SEQ ID NO: 28) |
| pAJW_P52 | gcattaagcgcggcgggtgtggtggttacgcgcagcgtgaccgctacacttgcca |
| | gcgccctagcgcccgctcctttcgctttcttcccttcctttctcgccacgttcgc |
| | cggctttccccgtcaagctctaaatcgggggctccctttagggttccgatttagt |
| | gctttacggcacctcgaccccaaaaaacttgattagggtgatggttcacgtagtg |
| | ggccatcgccctgatagacggtttttcgccctttgacgttggagtccacgttctt |
| | taatagtggactcttgttccaaactggaacaacactcaaccctatctcggtctat |
| | tcttttgatttataagggattttgccgatttcggcctattggttaaaaaatgagc |
| | tgatttaacaaaaatttaacgcgaattttaacaaaatattaacgcttacaattta |
| | ggtggcacttttcggggaaatgtgcgcggaacccctatttgtttatttttctaaa |
| | tacattcaaatatgtatccgctcatgagacaataaccctgataaatgcttcaata |
| | atattgaaaaaggaagagtatgagtattcaacatttccgtgtcgcccttattccc |
| | ttttttgcggcattttgccttcctgtttttgctcacccagaaacgctggtgaaag |
| | taaaagatgctgaagatcagttgggtgcacgagtgggttacatcgaactggatct |
| | caacagcggtaagatccttgagagttttcgccccgaagaacgttttccaatgatg |
| | agcacttttaaagttctgctatgtggcgcggtattatcccgtattgacgccgggc |
| | aagagcaactcggtcgccgcatacactattctcagaatgacttggttgagtactc |
| | accagtcacagaaaagcatcttacggatggcatgacagtaagagaattatgcagt |
| | gctgccataaccatgagtgataacactgcggccaacttacttctgacaacgatcg |
| | gaggaccgaaggagctaaccgcttttttgcacaacatgggggatcatgtaactcg |
| | ccttgatcgttgggaaccggagctgaatgaagccataccaaacgacgagcgtgac |
| | accacgatgcctgtagcaatggcaacaacgttgcgcaaactattaactggcgaac |
| | tacttactctagcttcccggcaacaattaatagactggatggaggcggataaagt |
| | tgcaggaccacttctgcgctcggcccttccggctggctggtttattgctgataaa |
| | tctggagccggtgagcgtgggtctcgcggtatcattgcagcactggggccagatg |
| | gtaagccctcccgtatcgtagttatctacacgacggggagtcaggcaactatgga |
| | tgaacgaaatagacagatcgctgagataggtgcctcactgattaagcattggtaa |
| | ctgtcagaccaagtttactcatatatactttagattgatttaaaacttcattttt |
| | aatttaaaaggatctaggtgaagatcctttttgataatctcatgaccaaaatccc |
| | ttaacgtgagttttcgttccactgagcgtcagaccccgtagaaaagatcaaagga |
| | tcttcttgagatcctttttttctgcgcgtaatctgctgcttgcaaacaaaaaaac |
| | caccgctaccagcggtggtttgtttgccggatcaagagctaccaactctttttcc |
| | gaaggtaactggcttcagcagagcgcagataccaaatactgttcttctagtgtag |
| | ccgtagttaggccaccacttcaagaactctgtagcaccgcctacatacctcgctc |
| | tgctaatcctgttaccagtggctgctgccagtggcgataagtcgtgtcttaccgg |
| | gttggactcaagacgatagttaccggataaggcgcagcggtcgggctgaacgggg |
| | ggttcgtgcacacagcccagcttggagcgaacgacctacaccgaactgagatacc |
| | tacagcgtgagctatgagaaagcgccacgcttcccgaagggagaaaggcggacag |
| | gtatccggtaagcggcagggtcggaacaggagagcgcacgagggagcttccaggg |
| | ggaaacgcctggtatctttatagtcctgtcgggtttcgccacctctgacttgagc |
| | gtcgatttttgtgatgctcgtcaggggggcggagcctatggaaaaacgccagcaa |
| | cgcggcctttttacggttcctggccttttgctggccttttgctcacatgttcttt |
| | cctgcgttatcccctgattctgtggataaccgtattaccgcctttgagtgagctg |
| | ataccgctcgccgcagccgaacgaccgagcgcagcgagtcagtgagcgaggaagc |
| | ggaagagcgcccaatacgcaaaccgcctctccccgcgcgttggccgattcattaa |
| | tgcagctggcacgacaggtttcccgactggaaagcgggcagtgagcgcaacgcaa |
| | ttaatgtgagttagctcactcattaggcaccccaggctttacactttatgcttcc |
| | ggctcgtatgttgtgtggaattgtgagcggataacaatttcacacaggaaacagc |
| | tatgaccatgattacgccaagctcgaaattaaccctcactaaagggaacaaaagc |
| | tggtacctcgcgcgacttggtttgccattctttagcgcgcgtcgcgtcacacagc |
| | ttggccacaatgtggtttttgtcaaacgaagattctatgacgtgtttaaagttta |
| | ggtcgagtaaagcgcaaatcttttttaaccctagaaagatagtctgcgtaaaatt |
| | gacgcatgcattcttgaaatattgctctctctttctaaatagcgcgaatccgtcg |
| | ctgtgcatttaggacatctcagtcgccgcttggagctcccgtgaggcgtgcttgt |
| | caatgcggtaagtgtcactgattttgaactataacgaccgcgtgagtcaaaatga |
| | cgcatgattatcttttacgtgacttttaagatttaactcatacgataattatatt |
| | gttatttcatgttctacttacgtgataacttattatatatatattttcttgttat |
| | agatatcatcaactttgtatagaaaagttgctcgacattgattattgactagtta |
| | ttaatagtaatcaattacggggtcattagttcatagcccatatatggagttccgc |
| | gttacataacttacggtaaatggcccgcctggctgaccgcccaacgacccccgcc |
| | cattgacgtcaataatgacgtatgttcccatagtaacgccaatagggactttcca |
| | ttgacgtcaatgggtggagtatttacggtaaactgcccacttggcagtacatcaa |
| | gtgtatcatatgccaagtacgccccctattgacgtcaatgacggtaaatggcccg |
| | cctggcattatgcccagtacatgaccttatgggactttcctacttggcagtacat |
| | ctacgtattagtcatcgctattaccatggtcgaggtgagccccacgttctgcttc |
| | actctccccatctcccccccctccccacccccaattttgtatttatttatttttt |
| | aattattttgtgcagcgatgggggcggggggggggggggggcgcgcgccaggcgg |
| | ggggggcggggcgaggggcggggggggcgaggcggagaggtgcggcggcagccaa |
| | tcagagcggcgcgctccgaaagtttccttttatggcgaggcggcggcggcggcgg |
| | ccctataaaaagcgaagcgcgcggcggggggagtcgctgcgcgctgccttcgccc |
| | cgtgccccgctccgccgccgcctcgcgccgcccgccccggctctgactgaccgcg |
| | ttactcccacaggtgagcggggggacggcccttctcctccgggctgtaattagcg |
| | cttggtttaatgacggcttgtttcttttctgtggctgcgtgaaagccttgagggg |
| | ctccgggagggccctttgtgcggggggagcggctcggggggtgcgtgcgtgtgtg |
| | tgtgcgtggggagcgccgcgtgcggctccgcgctgcccggcggctgtgagcgctg |
| | cgggcgcggcgcggggctttgtgcgctccgcagtgtgcgcgaggggagcgcggcc |
| | gggggcggtgccccgcggtgcggggggggctgcgaggggaacaaaggctgcgtgc |
| | ggggtgtgtgcgtgggggggtgagcagggggtgtgggcgcgtcggtcgggctgca |
| | accccccctgcacccccctccccgagttgctgagcacggcccggcttcgggtgcg |
| | gggctccgtacggggcgtggcgcggggctcgccgtgccgggcggggggtggcggc |
| | aggtgggggtgccgggcggggggggccgcctcgggccggggagggctcgggggag |
| | gggcgcggcggcccccggagcgccggcggctgtcgaggcgcggcgagccgcagcc |
| | attgccttttatggtaatcgtgcgagagggcgcagggacttcctttgtcccaaat |
| | ctgtgcggagccgaaatctgggaggcgccgccgcaccccctctagcgggcgcggg |
| | gcgaagcggtgcggcgccggcaggaaggaaatgggcggggagggccttcgtgcgt |
| | cgccgcgccgccgtccccttctccctctccagcctcggggctgtccgcgggggga |
| | cggctgccttcgggggggacggggcagggcggggttcggcttctggcgtgtgacc |
| | ggcggctctagagcctctgctaaccatgttcatgccttcttctttttcctacagc |
| | tcctgggcaacgtgctggttattgtgctgtctcatcattttggcaaagaattgca |
| | agtttgtacaaaaaagcaggctGAATGCCGCCACCATGGCTaccgagtataaacc |
| | gacggtccgacttgcgactcgggatgacgttccacgcgccgtgaggactctggcc |
| | gccgccttcgctgactacccggccacacggcacacagttgaccccgacagacaca |
| | tagagcgggtcacagagttgcaagaacttttcctgacgcgggttggActcgatat |
| | tggaaaagtttgggttgccgacgatggtgccgcagttgcggtgtggactactcca |
| | gaaagtgtagaagctggagccgtattcgctgaaatagggccacggatggcagagc |
| | tgtctggtagcaggctcgcagcgcagcagcagatggaaggCcttctcgctccgca |
| | tcggcctaaggaaccggcgtggttcctggctacggtgggagtctcacctgaccat |
| | caaggaaagggattgggaagtgctgtcgttcttccaggcgtggaagcggccgaac |
| | gagcaggcgtcccagccttcttggaAacgagcgcacccaggaatcttccattcta |
| | cgagcgactgggtttcactgtgacagcagacgtagaagtccctgaagggcctcgg |
| | acttggtgcatgaccagaaagcctggggcttgaAACGCCATgatccagacatgat |
| | aagatacattgatgagtttggacaaaccacaactagaatgcagtgaGGCGCGCCT |
| | GGATACTTCTGCCTGCAGGGGAGTCGCtcttttgcaatgctctttgggaaattat |
| | attagcctaattactaatttcctggccctcaaggtgacttgcttgaacttgccac |
| | ctTCGCCACTAGGGGGCAGCAttggtttacacagggtgaacaaacgttcaattct |
| | aaactaatattctttggtgggaaagtgtgttcattttggtttgttttgaagaagt |
| | cacttcatacctttgaactggggttaagctggttaactcccaaaattccaccagt |
| | tccaatatcctatggaaaaaccccaaaaccactCAAAccagatatactcgttgac |
| | attgattattgactagttattaatagtaatcaattacggggtcattagttcatag |
| | cccatatatggagttccgcgttacataacttacggtaaatggcccgcctggctga |
| | ccgcccaacgacccccgcccattgacgtcaataatgacgtatgttcccatagtaa |
| | cgccaatagggactttccattgacgtcaatgggtggagtatttacggtaaactgc |
| | ccacttggcagtacatcaagtgtatcatatgccaagtacgccccctattgacgtc |
| | aatgacggtaaatggcccgcctggcattatgcccagtacatgaccttatgggact |
| | ttcctacttggcagtacatctacgtattagtcatcgctattaccatggtgatgcg |
| | gttttggcagtacatcaatgggcgtggatagcggtttgactcacggggatttcca |
| | agtctccaccccattgacgtcaatgggagtttgttttggcaccaaaatcaacggg |
| | actttccaaaatgtcgtaacaactccgccccattgacgcaaatgggcggtaggcg |
| | tgtacggtgggaggtctatataagcagagctctctggctaactagagaacccact |
| | gcttactggcttatcgaaattaatacgactcactatagggagaaccaagcGAATG |
| | CCGCCACCATGCACAGTTCAGCTCTGCTGTGCTGCCTCGTACTTCTCACCGGCGT |
| | CAGAGCAAGCCCAGGCCAGGGCACCCAATCTGAGAATTCCTGTACACATTTCCCT |
| | GGGAATCTTCCCAATATGCTGCGCGACCTGCGGGATGCCTTCTCTCGCGTTAAGA |
| | CTTTTTTCCAGATGAAAGATCAGCTGGACAACCTCTTACTGAAGGAATCATTGCT |
| | CGAGGATTTTAAGGGATACTTAGGTTGCCAGGCGTTGTCCGAGATGATCCAGTTT |
| | TACCTGGAAGAGGTGATGCCTCAAGCAGAAAACCAAGACCCCGACATCAAAGCCC |
| | ACGTGAATAGCCTAGGAGAAAACTTGAAAACGCTCCGTCTGCGACTGCGGAGATG |
| | TCATAGGTTTCTACCATGTGAGAACAAAAGCAAGGCCGTCGAGCAGGTGAAGAAC |
| | GCCTTCAACAAACTTCAGGAGAAAGGGATTTATAAGGCTATGAGTGAATTCGATA |
| | TATTTATCAATTATATTGAGGCTTACATGACAATGAAGATTAGGAATTAAAACGC |
| | CATgggtggcatccctgtgacccctccccagtgcctctcctggccctggaagttg |
| | ccactccagtgcccaccagccttgtcctaataaaattaagttgcatcattttgtc |
| | tgactaggtgtccttctataatattatggggtggaggggggtggtatggagcaag |
| | gggcaagttgggaagagaacctgtagggcctgcggggtctattgggaaccaagct |
| | ggagtgcagtggcacaatcttggctcactgcaatctccgcctcctgggttcaagc |
| | gattctcctgcctcagcctcccgagttgttgggattccaggcatgcatgaccagg |
| | ctcagctaatttttgtttttttggtagagTcggggtttcaccatattggccaggc |
| | tggtgtccaactcctaatctcaggtgatctacccaccttggcctcccaaattgct |
| | gggattacaggcgtgaaccactgctcccttccctgtccttCAGTGGCATCGCtct |
| | tttgcaatgctctttgggaaattatattagcctaattactaatttcctggccctc |
| | aaggtgacttgcttgaacttgccacctTCGCCACTAGGGGGCAGCAttggtttac |
| | acagggtgaacaaacgttcaattctaaactaatattctttggtgggaaagtgtgt |
| | tcattttggtttgttttgaagaagtcacttcatacctttgaactggggttaagct |
| | ggttaactcccaaaattccaccagttccaatatcctatggaaaaaccccaaaacc |
| | actCAAAccagatatactcgttgacattgattattgactagttattaatagtaat |
| | caattacggggtcattagttcatagcccatatatggagttccgcgttacataact |
| | tacggtaaatggcccgcctggctgaccgcccaacgacccccgcccattgacgtca |
| | ataatgacgtatgttcccatagtaacgccaatagggactttccattgacgtcaat |
| | gggtggagtatttacggtaaactgcccacttggcagtacatcaagtgtatcatat |
| | gccaagtacgccccctattgacgtcaatgacggtaaatggcccgcctggcattat |
| | gcccagtacatgaccttatgggactttcctacttggcagtacatctacgtattag |
| | tcatcgctattaccatggtgatgcggttttggcagtacatcaatgggcgtggata |
| | gcggtttgactcacggggatttccaagtctccaccccattgacgtcaatgggagt |
| | ttgttttggcaccaaaatcaacgggactttccaaaatgtcgtaacaactccgccc |
| | cattgacgcaaatgggcggtaggcgtgtacggtgggaggtctatataagcagagc |
| | tctctggctaactagagaacccactgcttactggcttatcgaaattaatacgact |
| | cactatagggagaaccaagcGAATGCCGCCACCATGGCTgctgaaggTtccgtcg |
| | ccaggcagcctgacctcttgacctgcgacgatgagccgatccatatccccggtgc |
| | catccaaccgcatggactgctgctcgccctcgccgccgacatgacgatcgttgcc |
| | ggcagcgacaaccttcccgaactcaccggactggcgatcggcgccctgatcggcc |
| | gctctgcggccgatgtGttcgactcggaAacgcacaaccgtctgacgatcgcctt |
| | ggccgagcccggggcggccgtcggagcaccgatcactgtcggcttcacgatgcga |
| | aaggacgcaggcttcatcggctcctggcatcgccatgatcagctcatcttcctGg |
| | agctGgagcctccccagcgggacgtcgccgagccgcaggcgttcttccgccgcac |
| | caacagcgccatccgccgcctgcaggccgccgaaaccttggaaagcgcctgcgcc |
| | gccgcggcgcaagaggtgcggaagattaccggcttcgatcgggtgatgatctatc |
| | gcttcgcctccgacttcagcggcgaagtgatcgcagaggatcggtgcgccgaggt |
| | cgagtcaaaactaggcctgcactatcctgcctcaaccgtgccggcgcaggcccgt |
| | cggctctataccatcaacccggtacggatcattcccgatatcaattatcggccgg |
| | tgccggtcaccccagacctcaatccggtcaccgggcggccgattgatcttagctt |
| | cgccatcctgcgcagcgtGtcgcccgtccatctggaGttcatgcgcaacataggc |
| | atgcacggcacgatgtcgatctcgattttgcgcggcgagcgactgtggggattga |
| | tcgtttgccatcaccgaacgccgtactacgtcgatctcgatggccgccaagcctg |
| | cgagctagtcgcccaggttctggcctggcagatcggcgtgatggaagagTAAAAC |
| | GCCATgaattcactcctcaggtgcaggctgcctatcagaaggtggtggctggtgt |
| | ggccaatgccctggctcacaaataccactgagatctttttccctctgccaaaaat |
| | tatggggacatcatgaagccccttgagcatctgacttctggctaataaaggaaat |
| | ttattttcattgcaatagtgtgttggaattttttgtgtctctcactcggaaggac |
| | atatgggagggcaaatcatttaaaacatcagaatgagtatttggtttagagtttg |
| | gcaacatatgcccatatgctggctgccatgaacaaaggttggctataaagaggtc |
| | atcagtatatgaaacagccccctgctgtccattccttattccatagaaaagcctt |
| | gacttgaggttagattttttttatattttgttttgtgttatttttttctttaaca |
| | tccctaaaattttccttacatgttttactagccagatttttcctcctctcctgac |
| | tactcccagtcatagctgtccctcttctcttatggagatCAGTTCGCGCGGCCGC |
| | GCCTGGGCTTACttaattaacgagagcataatattgatatgtgccaaagttgttt |
| | ctgactgactaataagtataatttgtttctattatgtataggttaagctaattac |
| | ttattttataatacaacatgactgtttttaaagtacaaaataagtttatttttgt |
| | aaaagagagaatgtttaaaagttttgttactttatagaagaaattttgagttttt |
| | gtttttttttaataaataaataaacataaataaattgtttgttgaatttattatt |
| | agtatgtaagtgtaaatataataaaacttaatatctattcaaattaataaataaa |
| | cctcgatatacagaccgataaaacacatgcgtcaattttacgcatgattatcttt |
| | aacgtacgtcacaatatgattatctttctagggttaaataatagtttctaatttt |
| | tttattattcagcctgctgtcgtgaataccgagctccaattcgccctatagtgag |
| | tcgtattacaattcactggccgtcgttttacaacgtcgtgactgggaaaaccctg |
| | gcgttacccaacttaatcgccttgcagcacatccccctttcgccagctggcgtaa |
| | tagcgaagaggcccgcaccgatcgcccttcccaacagttgcgcagcctgaatggc |
| | gaatgggacgcgccctgtagcggc |
| | (SEQ ID NO: 29) |
| pAJW_P53 | gcattaagcgcggcgggtgtggtggttacgcgcagcgtgaccgctacacttgcca |
| | gcgccctagcgcccgctcctttcgctttcttcccttcctttctcgccacgttcgc |
| | cggctttccccgtcaagctctaaatcgggggctccctttagggttccgatttagt |
| | gctttacggcacctcgaccccaaaaaacttgattagggtgatggttcacgtagtg |
| | ggccatcgccctgatagacggtttttcgccctttgacgttggagtccacgttctt |
| | taatagtggactcttgttccaaactggaacaacactcaaccctatctcggtctat |
| | tcttttgatttataagggattttgccgatttcggcctattggttaaaaaatgagc |
| | tgatttaacaaaaatttaacgcgaattttaacaaaatattaacgcttacaattta |
| | ggtggcacttttcggggaaatgtgcgcggaacccctatttgtttatttttctaaa |
| | tacattcaaatatgtatccgctcatgagacaataaccctgataaatgcttcaata |
| | atattgaaaaaggaagagtatgagtattcaacatttccgtgtcgcccttattccc |
| | ttttttgcggcattttgccttcctgtttttgctcacccagaaacgctggtgaaag |
| | taaaagatgctgaagatcagttgggtgcacgagtgggttacatcgaactggatct |
| | caacagcggtaagatccttgagagttttcgccccgaagaacgttttccaatgatg |
| | agcacttttaaagttctgctatgtggcgcggtattatcccgtattgacgccgggc |
| | aagagcaactcggtcgccgcatacactattctcagaatgacttggttgagtactc |
| | accagtcacagaaaagcatcttacggatggcatgacagtaagagaattatgcagt |
| | gctgccataaccatgagtgataacactgcggccaacttacttctgacaacgatcg |
| | gaggaccgaaggagctaaccgcttttttgcacaacatgggggatcatgtaactcg |
| | ccttgatcgttgggaaccggagctgaatgaagccataccaaacgacgagcgtgac |
| | accacgatgcctgtagcaatggcaacaacgttgcgcaaactattaactggcgaac |
| | tacttactctagcttcccggcaacaattaatagactggatggaggcggataaagt |
| | tgcaggaccacttctgcgctcggcccttccggctggctggtttattgctgataaa |
| | tctggagccggtgagcgtgggtctcgcggtatcattgcagcactggggccagatg |
| | gtaagccctcccgtatcgtagttatctacacgacggggagtcaggcaactatgga |
| | tgaacgaaatagacagatcgctgagataggtgcctcactgattaagcattggtaa |
| | ctgtcagaccaagtttactcatatatactttagattgatttaaaacttcattttt |
| | aatttaaaaggatctaggtgaagatcctttttgataatctcatgaccaaaatccc |
| | ttaacgtgagttttcgttccactgagcgtcagaccccgtagaaaagatcaaagga |
| | tcttcttgagatcctttttttctgcgcgtaatctgctgcttgcaaacaaaaaaac |
| | caccgctaccagcggtggtttgtttgccggatcaagagctaccaactctttttcc |
| | gaaggtaactggcttcagcagagcgcagataccaaatactgttcttctagtgtag |
| | ccgtagttaggccaccacttcaagaactctgtagcaccgcctacatacctcgctc |
| | tgctaatcctgttaccagtggctgctgccagtggcgataagtcgtgtcttaccgg |
| | gttggactcaagacgatagttaccggataaggcgcagcggtcgggctgaacgggg |
| | ggttcgtgcacacagcccagcttggagcgaacgacctacaccgaactgagatacc |
| | tacagcgtgagctatgagaaagcgccacgcttcccgaagggagaaaggcggacag |
| | gtatccggtaagcggcagggtcggaacaggagagcgcacgagggagcttccaggg |
| | ggaaacgcctggtatctttatagtcctgtcgggtttcgccacctctgacttgagc |
| | gtcgatttttgtgatgctcgtcaggggggcggagcctatggaaaaacgccagcaa |
| | cgcggcctttttacggttcctggccttttgctggccttttgctcacatgttcttt |
| | cctgcgttatcccctgattctgtggataaccgtattaccgcctttgagtgagctg |
| | ataccgctcgccgcagccgaacgaccgagcgcagcgagtcagtgagcgaggaagc |
| | ggaagagcgcccaatacgcaaaccgcctctccccgcgcgttggccgattcattaa |
| | tgcagctggcacgacaggtttcccgactggaaagcgggcagtgagcgcaacgcaa |
| | ttaatgtgagttagctcactcattaggcaccccaggctttacactttatgcttcc |
| | ggctcgtatgttgtgtggaattgtgagcggataacaatttcacacaggaaacagc |
| | tatgaccatgattacgccaagctcgaaattaaccctcactaaagggaacaaaagc |
| | tggtacctcgcgcgacttggtttgccattctttagcgcgcgtcgcgtcacacagc |
| | ttggccacaatgtggtttttgtcaaacgaagattctatgacgtgtttaaagttta |
| | ggtcgagtaaagcgcaaatcttttttaaccctagaaagatagtctgcgtaaaatt |
| | gacgcatgcattcttgaaatattgctctctctttctaaatagcgcgaatccgtcg |
| | ctgtgcatttaggacatctcagtcgccgcttggagctcccgtgaggcgtgcttgt |
| | caatgcggtaagtgtcactgattttgaactataacgaccgcgtgagtcaaaatga |
| | cgcatgattatcttttacgtgacttttaagatttaactcatacgataattatatt |
| | gttatttcatgttctacttacgtgataacttattatatatatattttcttgttat |
| | agatatcatcaactttgtatagaaaagttgctcgacattgattattgactagtta |
| | ttaatagtaatcaattacggggtcattagttcatagcccatatatggagttccgc |
| | gttacataacttacggtaaatggcccgcctggctgaccgcccaacgacccccgcc |
| | cattgacgtcaataatgacgtatgttcccatagtaacgccaatagggactttcca |
| | ttgacgtcaatgggtggagtatttacggtaaactgcccacttggcagtacatcaa |
| | gtgtatcatatgccaagtacgccccctattgacgtcaatgacggtaaatggcccg |
| | cctggcattatgcccagtacatgaccttatgggactttcctacttggcagtacat |
| | ctacgtattagtcatcgctattaccatggtcgaggtgagccccacgttctgcttc |
| | actctccccatctcccccccctccccacccccaattttgtatttatttatttttt |
| | aattattttgtgcagcgatgggggcggggggggggggggggcgcgcgccaggcgg |
| | ggcggggcggggcgaggggcggggggggcgaggcggagaggtgcggcggcagcca |
| | atcagagcggcgcgctccgaaagtttccttttatggcgaggcggcggcggcggcg |
| | gccctataaaaagcgaagcgcgcggggggggagtcgctgcgcgctgccttcgccc |
| | cgtgccccgctccgccgccgcctcgcgccgcccgccccggctctgactgaccgcg |
| | ttactcccacaggtgagcggggggacggcccttctcctccgggctgtaattagcg |
| | cttggtttaatgacggcttgtttcttttctgtggctgcgtgaaagccttgagggg |
| | ctccgggagggccctttgtgcggggggagcggctcggggggtgcgtgcgtgtgtg |
| | tgtgcgtggggagcgccgcgtgcggctccgcgctgcccggcggctgtgagcgctg |
| | cgggcgcggcgcggggctttgtgcgctccgcagtgtgcgcgaggggagcgcggcc |
| | gggggcggtgccccgcggtgcggggggggctgcgaggggaacaaaggctgcgtgc |
| | ggggtgtgtgcgtgggggggtgagcagggggtgtgggcgcgtcggtcgggctgca |
| | accccccctgcacccccctccccgagttgctgagcacggcccggcttcgggtgcg |
| | gggctccgtacggggcgtggcgcggggctcgccgtgccgggcggggggtggcggc |
| | aggtgggggtgccgggcggggggggccgcctcgggccggggagggctcgggggag |
| | gggcgcggggcccccggagcgccggcggctgtcgaggcgcggcgagccgcagcca |
| | ttgccttttatggtaatcgtgcgagagggcgcagggacttcctttgtcccaaatc |
| | tgtgcggagccgaaatctgggaggcgccgccgcaccccctctagcgggcgcgggg |
| | cgaagcggtgcggcgccggcaggaaggaaatgggcggggagggccttcgtgcgtc |
| | gccgcgccgccgtccccttctccctctccagcctcggggctgtccgcggggggac |
| | ggctgccttcgggggggacggggcagggcggggttcggcttctggcgtgtgaccg |
| | gcggctctagagcctctgctaaccatgttcatgccttcttctttttcctacagct |
| | cctgggcaacgtgctggttattgtgctgtctcatcattttggcaaagaattgcaa |
| | gtttgtacaaaaaagcaggctGAATGCCGCCACCATGGCTaccgagtataaaccg |
| | acggtccgacttgcgactcgggatgacgttccacgcgccgtgaggactctggccg |
| | ccgccttcgctgactacccggccacacggcacacagttgaccccgacagacacat |
| | agagcgggtcacagagttgcaagaacttttcctgacgcgggttggActcgatatt |
| | ggaaaagtttgggttgccgacgatggtgccgcagttgcggtgtggactactccag |
| | aaagtgtagaagctggagccgtattcgctgaaatagggccacggatggcagagct |
| | gtctggtagcaggctcgcagcgcagcagcagatggaaggCcttctcgctccgcat |
| | cggcctaaggaaccggcgtggttcctggctacggtgggagtctcacctgaccatc |
| | aaggaaagggattgggaagtgctgtcgttcttccaggcgtggaagcggccgaacg |
| | agcaggcgtcccagccttcttggaAacgagcgcacccaggaatcttccattctac |
| | gagcgactgggtttcactgtgacagcagacgtagaagtccctgaagggcctcgga |
| | cttggtgcatgaccagaaagcctggggcttgaAACGCCATgatccagacatgata |
| | agatacattgatgagtttggacaaaccacaactagaatgcagtgaGGCGCGCCTG |
| | GATACTTCTGCCTGCAGGGGAGTCGCtcttttgcaatgctctttgggaaattata |
| | ttagcctaattactaatttcctggccctcaaggtgacttgcttgaacttgccacc |
| | tTCGCCACTAGGGGGCAGCAttggtttacacagggtgaacaaacgttcaattcta |
| | aactaatattctttggtgggaaagtgtgttcattttggtttgttttgaagaagtc |
| | acttcatacctttgaactggggttaagctggttaactcccaaaattccaccagtt |
| | ccaatatcctatggaaaaaccccaaaaccactCAAAggatctgcgatcgctccgg |
| | tgcccgtcagtgggcagagcgcacatcgcccacagtccccgagaagttgggggga |
| | ggggtcggcaattgaacgggtgcctagagaaggtggcgcggggtaaactgggaaa |
| | gtgatgtcgtgtactggctccgcctttttcccgaggggggggagaaccgtatata |
| | agtgcagtagtcgccgtgaacgttctttttcgcaacgggtttgccgccagaacac |
| | agctgaagcttcgaggggctcgcatctctccttcacgcgcccgccgccctacctg |
| | aggccgccatccacgccggttgagtcgcgttctgccgcctcccgcctgtggtgcc |
| | tcctgaactgcgtccgccgtctaggtaagtttaaagctcaggtcgagTccgggcc |
| | tttgtccggcgctcccttggagcctacctagactcagccggctctccacgctttg |
| | cctgaccctgcttgctcaactctacgtctttgtttcgttttctgttctgcgccgt |
| | tacagatccaagctgtgaccggcgcctacGAATGCCGCCACCATGCACAGTTCAG |
| | CTCTGCTGTGCTGCCTCGTACTTCTCACCGGCGTCAGAGCAAGCCCAGGCCAGGG |
| | CACCCAATCTGAGAATTCCTGTACACATTTCCCTGGGAATCTTCCCAATATGCTG |
| | CGCGACCTGCGGGATGCCTTCTCTCGCGTTAAGACTTTTTTCCAGATGAAAGATC |
| | AGCTGGACAACCTCTTACTGAAGGAATCATTGCTCGAGGATTTTAAGGGATACTT |
| | AGGTTGCCAGGCGTTGTCCGAGATGATCCAGTTTTACCTGGAAGAGGTGATGCCT |
| | CAAGCAGAAAACCAAGACCCCGACATCAAAGCCCACGTGAATAGCCTAGGAGAAA |
| | ACTTGAAAACGCTCCGTCTGCGACTGCGGAGATGTCATAGGTTTCTACCATGTGA |
| | GAACAAAAGCAAGGCCGTCGAGCAGGTGAAGAACGCCTTCAACAAACTTCAGGAG |
| | AAAGGGATTTATAAGGCTATGAGTGAATTCGATATATTTATCAATTATATTGAGG |
| | CTTACATGACAATGAAGATTAGGAATTAAAACGCCATgggtggcatccctgtgac |
| | ccctccccagtgcctctcctggccctggaagttgccactccagtgcccaccagcc |
| | ttgtcctaataaaattaagttgcatcattttgtctgactaggtgtccttctataa |
| | tattatggggggaggggggggtatggagcaaggggcaagttgggaagagaacctg |
| | tagggcctgcggggtctattgggaaccaagctggagtgcagtggcacaatcttgg |
| | ctcactgcaatctccgcctcctgggttcaagcgattctcctgcctcagcctcccg |
| | agttgttgggattccaggcatgcatgaccaggctcagctaatttttgtttttttg |
| | gtagagTcggggtttcaccatattggccaggctggtgtccaactcctaatctcag |
| | gtgatctacccaccttggcctcccaaattgctgggattacaggcgtgaaccactg |
| | ctcccttccctgtccttCAGTGGCATCGCtcttttgcaatgctctttgggaaatt |
| | atattagcctaattactaatttcctggccctcaaggtgacttgcttgaacttgcc |
| | acctTCGCCACTAGGGGGCAGCAttggtttacacagggtgaacaaacgttcaatt |
| | ctaaactaatattctttggtgggaaagtgtgttcattttggtttgttttgaagaa |
| | gtcacttcatacctttgaactggggttaagctggttaactcccaaaattccacca |
| | gttccaatatcctatggaaaaaccccaaaaccactCAAAccagatatactcgttg |
| | acattgattattgactagttattaatagtaatcaattacggggtcattagttcat |
| | agcccatatatggagttccgcgttacataacttacggtaaatggcccgcctggct |
| | gaccgcccaacgacccccgcccattgacgtcaataatgacgtatgttcccatagt |
| | aacgccaatagggactttccattgacgtcaatgggtggagtatttacggtaaact |
| | gcccacttggcagtacatcaagtgtatcatatgccaagtacgccccctattgacg |
| | tcaatgacggtaaatggcccgcctggcattatgcccagtacatgaccttatggga |
| | ctttcctacttggcagtacatctacgtattagtcatcgctattaccatggtgatg |
| | cggttttggcagtacatcaatgggcgtggatagcggtttgactcacggggatttc |
| | caagtctccaccccattgacgtcaatgggagtttgttttggcaccaaaatcaacg |
| | ggactttccaaaatgtcgtaacaactccgccccattgacgcaaatgggcggtagg |
| | cgtgtacggtgggaggtctatataagcagagctctctggctaactagagaaccca |
| | ctgcttactggcttatcgaaattaatacgactcactatagggagaaccaagcGAA |
| | TGCCGCCACCATGGCTgctgaaggTtccgtcgccaggcagcctgacctcttgacc |
| | tgcgacgatgagccgatccatatccccggtgccatccaaccgcatggactgctgc |
| | tcgccctcgccgccgacatgacgatcgttgccggcagcgacaaccttcccgaact |
| | caccggactggcgatcggcgccctgatcggccgctctgcggccgatgtGttcgac |
| | tcggaAacgcacaaccgtctgacgatcgccttggccgagcccggggcggccgtcg |
| | gagcaccgatcactgtcggcttcacgatgcgaaaggacgcaggcttcatcggctc |
| | ctggcatcgccatgatcagctcatcttcctGgagctGgagcctccccagcgggac |
| | gtcgccgagccgcaggcgttcttccgccgcaccaacagcgccatccgccgcctgc |
| | aggccgccgaaaccttggaaagcgcctgcgccgccgcggcgcaagaggtgcggaa |
| | gattaccggcttcgatcgggtgatgatctatcgcttcgcctccgacttcagcggc |
| | gaagtgatcgcagaggatcggtgcgccgaggtcgagtcaaaactaggcctgcact |
| | atcctgcctcaaccgtgccggcgcaggcccgtcggctctataccatcaacccggt |
| | acggatcattcccgatatcaattatcggccggtgccggtcaccccagacctcaat |
| | ccggtcaccgggcggccgattgatcttagcttcgccatcctgcgcagcgtGtcgc |
| | ccgtccatctggaGttcatgcgcaacataggcatgcacggcacgatgtcgatctc |
| | gattttgcgcggcgagcgactgtggggattgatcgtttgccatcaccgaacgccg |
| | tactacgtcgatctcgatggccgccaagcctgcgagctagtcgcccaggttctgg |
| | cctggcagatcggcgtgatggaagagTAAAACGCCATgaattcactcctcaggtg |
| | caggctgcctatcagaaggtggtggctggtgtggccaatgccctggctcacaaat |
| | accactgagatctttttccctctgccaaaaattatggggacatcatgaagcccct |
| | tgagcatctgacttctggctaataaaggaaatttattttcattgcaatagtgtgt |
| | tggaattttttgtgtctctcactcggaaggacatatgggagggcaaatcatttaa |
| | aacatcagaatgagtatttggtttagagtttggcaacatatgcccatatgctggc |
| | tgccatgaacaaaggttggctataaagaggtcatcagtatatgaaacagccccct |
| | gctgtccattccttattccatagaaaagccttgacttgaggttagatttttttta |
| | tattttgttttgtgttatttttttctttaacatccctaaaattttccttacatgt |
| | tttactagccagatttttcctcctctcctgactactcccagtcatagctgtccct |
| | cttctcttatggagatcCAGTTCGCGCGGCCGCGCCTGGGCTTACttaattaacg |
| | agagcataatattgatatgtgccaaagttgtttctgactgactaataagtataat |
| | ttgtttctattatgtataggttaagctaattacttattttataatacaacatgac |
| | tgtttttaaagtacaaaataagtttatttttgtaaaagagagaatgtttaaaagt |
| | tttgttactttatagaagaaattttgagtttttgtttttttttaataaataaata |
| | aacataaataaattgtttgttgaatttattattagtatgtaagtgtaaatataat |
| | aaaacttaatatctattcaaattaataaataaacctcgatatacagaccgataaa |
| | acacatgcgtcaattttacgcatgattatctttaacgtacgtcacaatatgatta |
| | tctttctagggttaaataatagtttctaatttttttattattcagcctgctgtcg |
| | tgaataccgagctccaattcgccctatagtgagtcgtattacaattcactggccg |
| | tcgttttacaacgtcgtgactgggaaaaccctggcgttacccaacttaatcgcct |
| | tgcagcacatccccctttcgccagctggcgtaatagcgaagaggcccgcaccgat |
| | cgcccttcccaacagttgcgcagcctgaatggcgaatgggacgcgccctgtagcg |
| | gc |
| | (SEQ ID NO: 30) |
| pAJW_P54 | gcattaagcgcggcgggtgtggtggttacgcgcagcgtgaccgctacacttgcca |
| | gcgccctagcgcccgctcctttcgctttcttcccttcctttctcgccacgttcgc |
| | cggctttccccgtcaagctctaaatcgggggctccctttagggttccgatttagt |
| | gctttacggcacctcgaccccaaaaaacttgattagggtgatggttcacgtagtg |
| | ggccatcgccctgatagacggtttttcgccctttgacgttggagtccacgttctt |
| | taatagtggactcttgttccaaactggaacaacactcaaccctatctcggtctat |
| | tcttttgatttataagggattttgccgatttcggcctattggttaaaaaatgagc |
| | tgatttaacaaaaatttaacgcgaattttaacaaaatattaacgcttacaattta |
| | ggtggcacttttcggggaaatgtgcgcggaacccctatttgtttatttttctaaa |
| | tacattcaaatatgtatccgctcatgagacaataaccctgataaatgcttcaata |
| | atattgaaaaaggaagagtatgagtattcaacatttccgtgtcgcccttattccc |
| | ttttttgcggcattttgccttcctgtttttgctcacccagaaacgctggtgaaag |
| | taaaagatgctgaagatcagttgggtgcacgagtgggttacatcgaactggatct |
| | caacagcggtaagatccttgagagttttcgccccgaagaacgttttccaatgatg |
| | agcacttttaaagttctgctatgtggcgcggtattatcccgtattgacgccgggc |
| | aagagcaactcggtcgccgcatacactattctcagaatgacttggttgagtactc |
| | accagtcacagaaaagcatcttacggatggcatgacagtaagagaattatgcagt |
| | gctgccataaccatgagtgataacactgcggccaacttacttctgacaacgatcg |
| | gaggaccgaaggagctaaccgcttttttgcacaacatgggggatcatgtaactcg |
| | ccttgatcgttgggaaccggagctgaatgaagccataccaaacgacgagcgtgac |
| | accacgatgcctgtagcaatggcaacaacgttgcgcaaactattaactggcgaac |
| | tacttactctagcttcccggcaacaattaatagactggatggaggcggataaagt |
| | tgcaggaccacttctgcgctcggcccttccggctggctggtttattgctgataaa |
| | tctggagccggtgagcgtgggtctcgcggtatcattgcagcactggggccagatg |
| | gtaagccccccgtatcgtagttatctacacgacggggagtcaggcaactatggat |
| | gaacgaaatagacagatcgctgagataggtgcctcactgattaagcattggtaac |
| | tgtcagaccaagtttactcatatatactttagattgatttaaaacttcattttta |
| | atttaaaaggatctaggtgaagatcctttttgataatctcatgaccaaaatccct |
| | taacgtgagttttcgttccactgagcgtcagaccccgtagaaaagatcaaaggat |
| | cttcttgagatcctttttttctgcgcgtaatctgctgcttgcaaacaaaaaaacc |
| | accgctaccagcggtggtttgtttgccggatcaagagctaccaactctttttccg |
| | aaggtaactggcttcagcagagcgcagataccaaatactgttcttctagtgtagc |
| | cgtagttaggccaccacttcaagaactctgtagcaccgcctacatacctcgctct |
| | gctaatcctgttaccagtggctgctgccagtggcgataagtcgtgtcttaccggg |
| | ttggactcaagacgatagttaccggataaggcgcagcggtcgggctgaacggggg |
| | gttcgtgcacacagcccagcttggagcgaacgacctacaccgaactgagatacct |
| | acagcgtgagctatgagaaagcgccacgcttcccgaagggagaaaggcggacagg |
| | tatccggtaagcggcagggtcggaacaggagagcgcacgagggagcttccagggg |
| | gaaacgcctggtatctttatagtcctgtcgggtttcgccacctctgacttgagcg |
| | tcgatttttgtgatgctcgtcaggggggcggagcctatggaaaaacgccagcaac |
| | gcggcctttttacggttcctggccttttgctggccttttgctcacatgttctttc |
| | ctgcgttatcccctgattctgtggataaccgtattaccgcctttgagtgagctga |
| | taccgctcgccgcagccgaacgaccgagcgcagcgagtcagtgagcgaggaagcg |
| | gaagagcgcccaatacgcaaaccgcctctccccgcgcgttggccgattcattaat |
| | gcagctggcacgacaggtttcccgactggaaagcgggcagtgagcgcaacgcaat |
| | taatgtgagttagctcactcattaggcaccccaggctttacactttatgcttccg |
| | gctcgtatgttgtgtggaattgtgagcggataacaatttcacacaggaaacagct |
| | atgaccatgattacgccaagctcgaaattaaccctcactaaagggaacaaaagct |
| | ggtacctcgcgcgacttggtttgccattctttagcgcgcgtcgcgtcacacagct |
| | tggccacaatgtggtttttgtcaaacgaagattctatgacgtgtttaaagtttag |
| | gtcgagtaaagcgcaaatcttttttaaccctagaaagatagtctgcgtaaaattg |
| | acgcatgcattcttgaaatattgctctctctttctaaatagcgcgaatccgtcgc |
| | tgtgcatttaggacatctcagtcgccgcttggagctcccgtgaggcgtgcttgtc |
| | aatgcggtaagtgtcactgattttgaactataacgaccgcgtgagtcaaaatgac |
| | gcatgattatcttttacgtgacttttaagatttaactcatacgataattatattg |
| | ttatttcatgttctacttacgtgataacttattatatatatattttcttgttata |
| | gatatcatcaactttgtatagaaaagttgctcgacattgattattgactagttat |
| | taatagtaatcaattacggggtcattagttcatagcccatatatggagttccgcg |
| | ttacataacttacggtaaatggcccgcctggctgaccgcccaacgacccccgccc |
| | attgacgtcaataatgacgtatgttcccatagtaacgccaatagggactttccat |
| | tgacgtcaatgggtggagtatttacggtaaactgcccacttggcagtacatcaag |
| | tgtatcatatgccaagtacgccccctattgacgtcaatgacggtaaatggcccgc |
| | ctggcattatgcccagtacatgaccttatgggactttcctacttggcagtacatc |
| | tacgtattagtcatcgctattaccatggtcgaggtgagccccacgttctgcttca |
| | ctctccccatctcccccccctccccacccccaattttgtatttatttatttttta |
| | attattttgtgcagcgatgggggcggggggggggggggggcgcgcgccaggcggg |
| | gggggcggggcgaggggcggggcggggcgaggcggagaggtgcggcggcagccaa |
| | tcagagcggcgcgctccgaaagtttccttttatggcgaggcggcggcggcggcgg |
| | ccctataaaaagcgaagcgcgcggcggggggagtcgctgcgcgctgccttcgccc |
| | cgtgccccgctccgccgccgcctcgcgccgcccgccccggctctgactgaccgcg |
| | ttactcccacaggtgagcggggggacggcccttctcctccgggctgtaattagcg |
| | cttggtttaatgacggcttgtttcttttctgtggctgcgtgaaagccttgagggg |
| | ctccgggagggccctttgtgcggggggagcggctcggggggtgcgtgcgtgtgtg |
| | tgtgcgtggggagcgccgcgtgcggctccgcgctgcccggcggctgtgagcgctg |
| | cgggcgcggcgcggggctttgtgcgctccgcagtgtgcgcgaggggagcgcggcc |
| | gggggcggtgccccgcggtgcggggggggctgcgaggggaacaaaggctgcgtgc |
| | ggggtgtgtgcgtgggggggtgagcagggggtgtgggcgcgtcggtcgggctgca |
| | accccccctgcacccccctccccgagttgctgagcacggcccggcttcgggtgcg |
| | gggctccgtacggggcgtggcgcggggctcgccgtgccgggcggggggtggcggc |
| | aggtgggggtgccgggcggggcggggccgcctcgggccggggagggctcggggga |
| | ggggcgcggcggcccccggagcgccggcggctgtcgaggcgcggcgagccgcagc |
| | cattgccttttatggtaatcgtgcgagagggcgcagggacttcctttgtcccaaa |
| | tctgtgcggagccgaaatctgggaggcgccgccgcaccccctctagcgggcgcgg |
| | ggcgaagcggtgcggcgccggcaggaaggaaatgggcggggagggccttcgtgcg |
| | tcgccgcgccgccgtccccttctccctctccagcctcggggctgtccgcgggggg |
| | acggctgccttcgggggggacggggcagggcggggttcggcttctggcgtgtgac |
| | cggcggctctagagcctctgctaaccatgttcatgccttcttctttttcctacag |
| | ctcctgggcaacgtgctggttattgtgctgtctcatcattttggcaaagaattgc |
| | aagtttgtacaaaaaagcaggctGAATGCCGCCACCATGGCTaccgagtataaac |
| | cgacggtccgacttgcgactcgggatgacgttccacgcgccgtgaggactctggc |
| | cgccgccttcgctgactacccggccacacggcacacagttgaccccgacagacac |
| | atagagcgggtcacagagttgcaagaacttttcctgacgcgggttggActcgata |
| | ttggaaaagtttgggttgccgacgatggtgccgcagttgcggtgtggactactcc |
| | agaaagtgtagaagctggagccgtattcgctgaaatagggccacggatggcagag |
| | ctgtctggtagcaggctcgcagcgcagcagcagatggaaggCcttctcgctccgc |
| | atcggcctaaggaaccggcgtggttcctggctacggtgggagtctcacctgacca |
| | tcaaggaaagggattgggaagtgctgtcgttcttccaggcgtggaagcggccgaa |
| | cgagcaggcgtcccagccttcttggaAacgagcgcacccaggaatcttccattct |
| | acgagcgactgggtttcactgtgacagcagacgtagaagtccctgaagggcctcg |
| | gacttggtgcatgaccagaaagcctggggcttgaAACGCCATgatccagacatga |
| | taagatacattgatgagtttggacaaaccacaactagaatgcagtgaGGCGCGCC |
| | TGGATACTTCTGCCTGCAGGGGAGTCGCtcttttgcaatgctctttgggaaatta |
| | tattagcctaattactaatttcctggccctcaaggtgacttgcttgaacttgcca |
| | cctTCGCCACTAGGGGGCAGCAttggtttacacagggtgaacaaacgttcaattc |
| | taaactaatattctttggtgggaaagtgtgttcattttggtttgttttgaagaag |
| | tcacttcatacctttgaactggggttaagctggttaactcccaaaattccaccag |
| | ttccaatatcctatggaaaaaccccaaaaccactCAAAGAATTCtaccgggtagg |
| | ggaggcgcttttcccaaggcagtctggagcatgcgctttagcagcAccgctgggc |
| | acttggcgctacacaagtggcctctggcctcgcacacattccacatccaccggta |
| | ggcgccaaccggctccgttctttggtggccccttcgcgccaccttctactcctcc |
| | cctagtcaggaagttccccTccgccccgcagctcgcgtcgtgcaggacgtgacaa |
| | atggaagtagcacgtctAactagtctcgtgcagatggacagcaccgctgagcaat |
| | ggaagcgggtaggcctttggggcagcggccaatagcagctttgctccttcgcttt |
| | ctgggctcaggggggggcgggcgcccgaaggtcctccggaggcccggcattctgc |
| | acgcttcaaaagcgcacgtctgccgcgctgttctcctcttcctcatctccgggcc |
| | tttcgGAATGCCGCCACCATGCACAGTTCAGCTCTGCTGTGCTGCCTCGTACTTC |
| | TCACCGGCGTCAGAGCAAGCCCAGGCCAGGGCACCCAATCTGAGAATTCCTGTAC |
| | ACATTTCCCTGGGAATCTTCCCAATATGCTGCGCGACCTGCGGGATGCCTTCTCT |
| | CGCGTTAAGACTTTTTTCCAGATGAAAGATCAGCTGGACAACCTCTTACTGAAGG |
| | AATCATTGCTCGAGGATTTTAAGGGATACTTAGGTTGCCAGGCGTTGTCCGAGAT |
| | GATCCAGTTTTACCTGGAAGAGGTGATGCCTCAAGCAGAAAACCAAGACCCCGAC |
| | ATCAAAGCCCACGTGAATAGCCTAGGAGAAAACTTGAAAACGCTCCGTCTGCGAC |
| | TGCGGAGATGTCATAGGTTTCTACCATGTGAGAACAAAAGCAAGGCCGTCGAGCA |
| | GGTGAAGAACGCCTTCAACAAACTTCAGGAGAAAGGGATTTATAAGGCTATGAGT |
| | GAATTCGATATATTTATCAATTATATTGAGGCTTACATGACAATGAAGATTAGGA |
| | ATTAAAACGCCATgggtggcatccctgtgacccctccccagtgcctctcctggcc |
| | ctggaagttgccactccagtgcccaccagccttgtcctaataaaattaagttgca |
| | tcattttgtctgactaggtgtccttctataatattatggggtggaggggggtggt |
| | atggagcaaggggcaagttgggaagagaacctgtagggcctgcggggtctattgg |
| | gaaccaagctggagtgcagtggcacaatcttggctcactgcaatctccgcctcct |
| | gggttcaagcgattctcctgcctcagcctcccgagttgttgggattccaggcatg |
| | catgaccaggctcagctaatttttgtttttttggtagagTcggggtttcaccata |
| | ttggccaggctggtgtccaactcctaatctcaggtgatctacccaccttggcctc |
| | ccaaattgctgggattacaggcgtgaaccactgctcccttccctgtccttCAGTG |
| | GCATCGCtcttttgcaatgctctttgggaaattatattagcctaattactaattt |
| | cctggccctcaaggtgacttgcttgaacttgccacctTCGCCACTAGGGGGCAGC |
| | Attggtttacacagggtgaacaaacgttcaattctaaactaatattctttggtgg |
| | gaaagtgtgttcattttggtttgttttgaagaagtcacttcatacctttgaactg |
| | gggttaagctggttaactcccaaaattccaccagttccaatatcctatggaaaaa |
| | ccccaaaaccactCAAAccagatatactcgttgacattgattattgactagttat |
| | taatagtaatcaattacggggtcattagttcatagcccatatatggagttccgcg |
| | ttacataacttacggtaaatggcccgcctggctgaccgcccaacgacccccgccc |
| | attgacgtcaataatgacgtatgttcccatagtaacgccaatagggactttccat |
| | tgacgtcaatgggtggagtatttacggtaaactgcccacttggcagtacatcaag |
| | tgtatcatatgccaagtacgccccctattgacgtcaatgacggtaaatggcccgc |
| | ctggcattatgcccagtacatgaccttatgggactttcctacttggcagtacatc |
| | tacgtattagtcatcgctattaccatggtgatgcggttttggcagtacatcaatg |
| | ggcgtggatagcggtttgactcacggggatttccaagtctccaccccattgacgt |
| | caatgggagtttgttttggcaccaaaatcaacgggactttccaaaatgtcgtaac |
| | aactccgccccattgacgcaaatgggcggtaggcgtgtacggtgggaggtctata |
| | taagcagagctctctggctaactagagaacccactgcttactggcttatcgaaat |
| | taatacgactcactatagggagaaccaagcGAATGCCGCCACCATGGCTgctgaa |
| | ggTtccgtcgccaggcagcctgacctcttgacctgcgacgatgagccgatccata |
| | tccccggtgccatccaaccgcatggactgctgctcgccctcgccgccgacatgac |
| | gatcgttgccggcagcgacaaccttcccgaactcaccggactggcgatcggcgcc |
| | ctgatcggccgctctgcggccgatgtGttcgactcggaAacgcacaaccgtctga |
| | cgatcgccttggccgagcccggggggccgtcggagcaccgatcactgtcggcttc |
| | acgatgcgaaaggacgcaggcttcatcggctcctggcatcgccatgatcagctca |
| | tcttcctGgagctGgagcctccccagcgggacgtcgccgagccgcaggcgttctt |
| | ccgccgcaccaacagcgccatccgccgcctgcaggccgccgaaaccttggaaagc |
| | gcctgcgccgccgcggcgcaagaggtgcggaagattaccggcttcgatcgggtga |
| | tgatctatcgcttcgcctccgacttcagcggcgaagtgatcgcagaggatcggtg |
| | cgccgaggtcgagtcaaaactaggcctgcactatcctgcctcaaccgtgccggcg |
| | caggcccgtcggctctataccatcaacccggtacggatcattcccgatatcaatt |
| | atcggccggtgccggtcaccccagacctcaatccggtcaccgggcggccgattga |
| | tcttagcttcgccatcctgcgcagcgtGtcgcccgtccatctggaGttcatgcgc |
| | aacataggcatgcacggcacgatgtcgatctcgattttgcgcggcgagcgactgt |
| | ggggattgatcgtttgccatcaccgaacgccgtactacgtcgatctcgatggccg |
| | ccaagcctgcgagctagtcgcccaggttctggcctggcagatcggcgtgatggaa |
| | gagTAAAACGCCATgaattcactcctcaggtgcaggctgcctatcagaaggtggt |
| | ggctggtgtggccaatgccctggctcacaaataccactgagatctttttccctct |
| | gccaaaaattatggggacatcatgaagccccttgagcatctgacttctggctaat |
| | aaaggaaatttattttcattgcaatagtgtgttggaattttttgtgtctctcact |
| | cggaaggacatatgggagggcaaatcatttaaaacatcagaatgagtatttggtt |
| | tagagtttggcaacatatgcccatatgctggctgccatgaacaaaggttggctat |
| | aaagaggtcatcagtatatgaaacagccccctgctgtccattccttattccatag |
| | aaaagccttgacttgaggttagattttttttatattttgttttgtgttatttttt |
| | tctttaacatccctaaaattttccttacatgttttactagccagatttttcctcc |
| | tctcctgactactcccagtcatagctgtccctcttctcttatggagatcCAGTTC |
| | GCGCGGCCGCGCCTGGGCTTACttaattaacgagagcataatattgatatgtgcc |
| | aaagttgtttctgactgactaataagtataatttgtttctattatgtataggtta |
| | agctaattacttattttataatacaacatgactgtttttaaagtacaaaataagt |
| | ttatttttgtaaaagagagaatgtttaaaagttttgttactttatagaagaaatt |
| | ttgagtttttgtttttttttaataaataaataaacataaataaattgtttgttga |
| | atttattattagtatgtaagtgtaaatataataaaacttaatatctattcaaatt |
| | aataaataaacctcgatatacagaccgataaaacacatgcgtcaattttacgcat |
| | gattatctttaacgtacgtcacaatatgattatctttctagggttaaataatagt |
| | ttctaatttttttattattcagcctgctgtcgtgaataccgagctccaattcgcc |
| | ctatagtgagtcgtattacaattcactggccgtcgttttacaacgtcgtgactgg |
| | gaaaaccctggcgttacccaacttaatcgccttgcagcacatccccctttcgcca |
| | gctggcgtaatagcgaagaggcccgcaccgatcgcccttcccaacagttgcgcag |
| | cctgaatggcgaatgggacgcgccctgtagcggc |
| | (SEQ ID NO: 31) |
| pOCF_Ros AB | tagcaggcatgctggggatgcggtgggctctatggcttctgaggcggaaagaacc |
| | agctggggctctagggggtatccccacgcgccctgtagcggcgcattaagcgcgg |
| | cgggtgtggtggttacgcgcagcgtgaccgctacacttgccagcgccctagcgcc |
| | cgctcctttcgctttcttcccttcctttctcgccacgttcgccggctttccccgt |
| | caagctctaaatcgggggctccctttagggttccgatttagtgctttacggcacc |
| | tcgaccccaaaaaacttgattagggtgatggttcacgtagtgggccatcgccctg |
| | atagacggtttttcgccctttgacgttggagtccacgttctttaatagtggactc |
| | ttgttccaaactggaacaacactcaaccctatctcggtctattcttttgatttat |
| | aagggattttgccgatttcggcctattggttaaaaaatgagctgatttaacaaaa |
| | atttaacgcgaattaattctgtggaatgtgtgtcagttagggtgtggaaagtccc |
| | caggctccccagcaggcagaagtatgcaaagcatgcatctcaattagtcagcaac |
| | caggtgtggaaagtccccaggctccccagcaggcagaagtatgcaaagcatgcat |
| | ctcaattagtcagcaaccatagtcccgcccctaactccgcccatcccgcccctaa |
| | ctccgcccagttccgcccattctccgccccatggctgactaattttttttattta |
| | tgcagaggccgaggccgcctctgcctctgagctattccagaagtagtgaggaggc |
| | ttttttggaggcctaggcttttgcaaaaagctcccgggagcttgtatatccattt |
| | tcggatctgatcagcacgtgttgacaattaatcatcggcatagtatatcggcata |
| | gtataatacgacaaggtgaggaactaaaccatggccaagttgaccagtgccgttc |
| | cggtgctcaccgcgcgcgacgtcgccggagcggtcgagttctggaccgaccggct |
| | cgggttctcccgggacttcgtggaggacgacttcgccggtgtggtccgggacgac |
| | gtgaccctgttcatcagcgcggtccaggaccaggtggtgccggacaacaccctgg |
| | cctgggtgtgggtgcgcggcctggacgagctgtacgccgagtggtcggaggtcgt |
| | gtccacgaacttccgggacgcctccgggccggccatgaccgagatcggcgagcag |
| | ccgtgggggcgggagttcgccctgcgcgacccggccggcaactgcgtgcacttcg |
| | tggccgaggagcaggactgacacgtgctacgagatttcgattccaccgccgcctt |
| | ctatgaaaggttgggcttcggaatcgttttccgggacgccggctggatgatcctc |
| | cagcgcggggatctcatgctggagttcttcgcccaccccaacttgtttattgcag |
| | cttataatggttacaaataaagcaatagcatcacaaatttcacaaataaagcatt |
| | tttttcactgcattctagttgtggtttgtccaaactcatcaatgtatcttatcat |
| | gtctgtataccgtcgacctctagctagagcttggcgtaatcatggtcatagctgt |
| | ttcctgtgtgaaattgttatccgctcacaattccacacaacatacgagccggaag |
| | cataaagtgtaaagcctggggtgcctaatgagtgagctaactcacattaattgcg |
| | ttgcgctcactgcccgctttccagtcgggaaacctgtcgtgccagctgcattaat |
| | gaatcggccaacgcgcggggagaggcggtttgcgtattgggcgctcttccgcttc |
| | ctcgctcactgactcgctgcgctcggtcgttcggctgcggcgagcggtatcagct |
| | cactcaaaggcggtaatacggttatccacagaatcaggggataacgcaggaaaga |
| | acatgtgagcaaaaggccagcaaaaggccaggaaccgtaaaaaggccgcgttgct |
| | ggcgtttttccataggctccgcccccctgacgagcatcacaaaaatcgacgctca |
| | agtcagaggtggcgaaacccgacaggactataaagataccaggcgtttccccctg |
| | gaagctccctcgtgcgctctcctgttccgaccctgccgcttaccggatacctgtc |
| | cgcctttctcccttcgggaagcgtggcgctttctcatagctcacgctgtaggtat |
| | ctcagttcggtgtaggtcgttcgctccaagctgggctgtgtgcacgaaccccccg |
| | ttcagcccgaccgctgcgccttatccggtaactatcgtcttgagtccaacccggt |
| | aagacacgacttatcgccactggcagcagccactggtaacaggattagcagagcg |
| | aggtatgtaggcggtgctacagagttcttgaagtggtggcctaactacggctaca |
| | ctagaagaacagtatttggtatctgcgctctgctgaagccagttaccttcggaaa |
| | aagagttggtagctcttgatccggcaaacaaaccaccgctggtagcggtggtttt |
| | tttgtttgcaagcagcagattacgcgcagaaaaaaaggatctcaagaagatcctt |
| | tgatcttttctacggggtctgacgctcagtggaacgaaaactcacgttaagggat |
| | tttggtcatgagattatcaaaaaggatcttcacctagatccttttaaattaaaaa |
| | tgaagttttaaatcaatctaaagtatatatgagtaaacttggtctgacagttacc |
| | aatgcttaatcagtgaggcacctatctcagcgatctgtctatttcgttcatccat |
| | agttgcctgactccccgtcgtgtagataactacgatacgggagggcttaccatct |
| | ggccccagtgctgcaatgataccgcgagacccacgctcaccggctccagatttat |
| | cagcaataaaccagccagccggaagggccgagcgcagaagtggtcctgcaacttt |
| | atccgcctccatccagtctattaattgttgccgggaagctagagtaagtagttcg |
| | ccagttaatagtttgcgcaacgttgttgccattgctacaggcatcgtggtgtcac |
| | gctcgtcgtttggtatggcttcattcagctccggttcccaacgatcaaggcgagt |
| | tacatgatcccccatgttgtgcaaaaaagcggttagctccttcggtcctccgatc |
| | gttgtcagaagtaagttggccgcagtgttatcactcatggttatggcagcactgc |
| | ataattctcttactgtcatgccatccgtaagatgcttttctgtgactggtgagta |
| | ctcaaccaagtcattctgagaatagtgtatgcggcgaccgagttgctcttgcccg |
| | gcgtcaatacgggataataccgcgccacatagcagaactttaaaagtgctcatca |
| | ttggaaaacgttcttcggggcgaaaactctcaaggatcttaccgctgttgagatc |
| | cagttcgatgtaacccactcgtgcacccaactgatcttcagcatcttttactttc |
| | accagcgtttctgggtgagcaaaaacaggaaggcaaaatgccgcaaaaaagggaa |
| | taagggcgacacggaaatgttgaatactcatactcttcctttttcaatattattg |
| | aagcatttatcagggttattgtctcatgagcggatacatatttgaatgtatttag |
| | aaaaataaacaaataggggttccgcgcacatttccccgaaaagtgccacctgacg |
| | tcgacggatcgggagatctcccgatcccctatggtgcactctcagtacaatctgc |
| | tctgatgccgcatagttaagccagtatctgctccctgcttgtgtgttggaggtcg |
| | ctgagtagtgcgcgagcaaaatttaagctacaacaaggcaaggcttgaccgacaa |
| | ttgcatgaagaatctgcttagggttaggcgttttgcgctgcttcgcgatgtacgg |
| | gccagatatacgcgttgacattgattattgactagttattaatagtaatcaatta |
| | cggggtcattagttcatagcccatatatggagttccgcgttacataacttacggt |
| | aaatggcccgcctggctgaccgcccaacgacccccgcccattgacgtcaataatg |
| | acgtatgttcccatagtaacgccaatagggactttccattgacgtcaatgggtgg |
| | agtatttacggtaaactgcccacttggcagtacatcaagtgtatcatatgccaag |
| | tacgccccctattgacgtcaatgacggtaaatggcccgcctggcattatgcccag |
| | tacatgaccttatgggactttcctacttggcagtacatctacgtattagtcatcg |
| | ctattaccatggtgatgcggttttggcagtacatcaatgggcgtggatagcggtt |
| | tgactcacggggatttccaagtctccaccccattgacgtcaatgggagtttgttt |
| | tggcaccaaaatcaacgggactttccaaaatgtcgtaacaactccgccccattga |
| | cgcaaatgggcggtaggcgtgtacggtgggaggtctatataagcagcgcgttttg |
| | cctgtactgggtctctctggttagaccagatctgagcctgggagctctctggcta |
| | actagggaacccactgcttaagcctcaataaagcttgccttgagtgcttcaagta |
| | gtgtgtgcccgtctgttgtgtgactctggtaactagagatccctcagaccctttt |
| | agtcagtgtggaaaatctctagcagtggcgcccgaacagggacttgaaagcgaaa |
| | gggaaaccagaggagctctctcgacgcaggactcggcttgctgaagcgcgcacgg |
| | caagaggcgaggggcggcgactggtgagtacgccaaaaattttgactagcggagg |
| | ctagaaggagagagatgggtgcgagagcgtcagtattaagcgggggagaattaga |
| | tcgcgatgggaaaaaattcggttaaggccagggggaaagaaaaaatataaattaa |
| | aacatatagtatgggcaagcagggagctagaacgattcgcagttaatcctggcct |
| | gttagaaacatcagaaggctgtagacaaatactgggacagctacaaccatccctt |
| | cagacaggatcagaagaacttagatcattatataatacagtagcaaccctctatt |
| | gtgtgcatcaaaggatagagataaaagacaccaaggaagctttagacaagataga |
| | ggaagagcaaaacaaaagtaagaccaccgcacagcaagcggccgctgatcttcag |
| | acctggaggaggagatatgagggacaattggagaagtgaattatataaatataaa |
| | gtagtaaaaattgaaccattaggagtagcacccaccaaggcaaagagaagagtgg |
| | tgcagagagaaaaaagagcagtgggaataggagctttgttccttgggttcttggg |
| | agcagcaggaagcactatgggcgcagcgtcaatgacgctgacggtacaggccaga |
| | caattattgtctggtatagtgcagcagcagaacaatttgctgagggctattgagg |
| | cgcaacagcatctgttgcaactcacagtctggggcatcaagcagctccaggcaag |
| | aatcctggctgtggaaagatacctaaaggatcaacagctcctggggatttggggt |
| | tgctctggaaaactcatttgcaccactgctgtgccttggaatgctagttggagta |
| | ataaatctctggaacagatttggaatcacacgacctggatggagtgggacagaga |
| | aattaacaattacacaagcttaatacactccttaattgaagaatcgcaaaaccag |
| | caagaaaagaatgaacaagaattattggaattagataaatgggcaagtttgtgga |
| | attggtttaacataacaaattggctgtggtatataaaattattcataatgatagt |
| | aggaggcttggtaggtttaagaatagtttttgctgtactttctatagtgaataga |
| | gttaggcagggatattcaccattatcgtttcagacccacctcccaaccccgaggg |
| | gacccgacaggcccgaaggaatagaagaagaaggtggagagagagacagagacag |
| | atccattcgattagtgaacggatcggcactgcgtgcgccaattctgcagacaaat |
| | ggcagtattcatccacaattttaaaagaaaaggggggattggggggtacagtgca |
| | ggggaaagaatagtagacataatagcaacagacatacaaactaaagaattacaaa |
| | aacaaattacaaaaattcaaaattttcgggtttattacagggacagcagagatcc |
| | agtttggttaattaaggtaccGGAGatGTCTTCCGTTgctagccctgcacgCTTC |
| | tttacagctagctcagtcctaggtattatgctagctactCGACctagtgAAAGAG |
| | GAGAAAtactaAATGgtgagtaaaggagaagaaaacaacttagctatcattaaag |
| | agttcatgcgcttcaaagttcacatggagggttctgttaacggtcacgagttcga |
| | gatcgaaggcgaaggcgagggccgtccgtatgaaggcacccagaccgccaaactg |
| | aaagtgactaaaggcggcccgctgccttttgcgtgggacatcctgagcccgcaat |
| | ttatgtacggttctaaagcgtatgttaaacacccagcggatatcccggactatct |
| | gaagctgtcttttccggaaggtttcaactgggaacgcgtaatgaattttgaagat |
| | ggtggtgtcgtgaccgtcactcaggactcctccctTcaggatggcgagttcatct |
| | ataaagttaaactgcgtggtactaattttccatctgatggcccggtgatgcagtg |
| | taggacgatgggttgggaggcgtctaccgaacgcatgtatccggaagatggtgcg |
| | ctgaaaggcgaaattaaacagcgcctgaaactgaaagatggcggccattatgacg |
| | ctgaagtgaaaaccacgtacaaagccaagaaacctgtgcagctgcctggcgcgta |
| | caatgtggatattaaactggacatcttatctcataatgaagattatacgatcgta |
| | gagcaatatgagcgcgcggagggtcgtcattctaccggtggcatggatgaactat |
| | acaaataaTGGCctcgcgtacgcaaagacgaacaataagacgctgaaaagcgtct |
| | tttttcgttttggtccGCTGTTCCtGAAGACatGGCAtaagtcgacaatcaacct |
| | ctggattacaaaatttgtgaaagattgactggtattcttaactatgttgctcctt |
| | ttacgctatgtggatacgctgctttaatgcctttgtatcatgctattgcttcccg |
| | tatggctttcattttctcctccttgtataaatcctggttgctgtctctttatgag |
| | gagttgtggcccgttgtcaggcaacgtggcgtggtgtgcactgtgtttgctgacg |
| | caacccccactggttggggcattgccaccacctgtcagctcctttccgggacttt |
| | cgctttccccctccctattgccacggcggaactcatcgccgcctgccttgcccgc |
| | tgctggacaggggctcggctgttgggcactgacaattccgtggtgttgtcgggga |
| | aatcatcgtcctttccttggctgctcgcctgtgttgccacctggattctgcgcgg |
| | gacgtccttctgctacgtcccttcggccctcaatccagcggaccttccttcccgc |
| | ggcctgctgccggctctgcggcctcttccgcgtctAcgccttcgccctcagacga |
| | gtcggatctccctttgggccgcctccccgcgtcgactttaagaccaatgacttac |
| | aaggcagctgtagatcttagccactttttaaaagaaaaggggggactggaagggc |
| | taattcactcccaacgaagTcaagatctgctttttgcttgtactgggtctctctg |
| | gttagaccagatctgagcctgggagctctctggctaactagggaacccactgctt |
| | aagcctcaataaagcttgccttgagtgcttcaagtagtgtgtgcccgtctgttgt |
| | gtgactctggtaactagagatccctcagacccttttagtcagtgtggaaaatctc |
| | tagcagggcccgtttaaacccgctgatcagcctcgactgtgccttctagttgcca |
| | gccatctgttgtttgcccctcccccgtgccttccttgaccctggaaggtgccact |
| | cccactgtcctttcctaataaaatgaggaaattgcatcgcattgtctgagtaggt |
| | gtcattctattctggggggtggggggggcaggacagcaagggggaggattgggaa |
| | gagaa |
| | (SEQ ID NO: 32) |
| pOCF_Ros AC | tagcaggcatgctggggatgcggtgggctctatggcttctgaggcggaaagaacc |
| | agctggggctctagggggtatccccacgcgccctgtagcggcgcattaagcgcgg |
| | cgggtgtggtggttacgcgcagcgtgaccgctacacttgccagcgccctagcgcc |
| | cgctcctttcgctttcttcccttcctttctcgccacgttcgccggctttccccgt |
| | caagctctaaatcgggggctccctttagggttccgatttagtgctttacggcacc |
| | tcgaccccaaaaaacttgattagggtgatggttcacgtagtgggccatcgccctg |
| | atagacggtttttcgccctttgacgttggagtccacgttctttaatagtggactc |
| | ttgttccaaactggaacaacactcaaccctatctcggtctattcttttgatttat |
| | aagggattttgccgatttcggcctattggttaaaaaatgagctgatttaacaaaa |
| | atttaacgcgaattaattctgtggaatgtgtgtcagttagggtgtggaaagtccc |
| | caggctccccagcaggcagaagtatgcaaagcatgcatctcaattagtcagcaac |
| | caggtgtggaaagtccccaggctccccagcaggcagaagtatgcaaagcatgcat |
| | ctcaattagtcagcaaccatagtcccgcccctaactccgcccatcccgcccctaa |
| | ctccgcccagttccgcccattctccgccccatggctgactaattttttttattta |
| | tgcagaggccgaggccgcctctgcctctgagctattccagaagtagtgaggaggc |
| | ttttttggaggcctaggcttttgcaaaaagctcccgggagcttgtatatccattt |
| | tcggatctgatcagcacgtgttgacaattaatcatcggcatagtatatcggcata |
| | gtataatacgacaaggtgaggaactaaaccatggccaagttgaccagtgccgttc |
| | cggtgctcaccgcgcgcgacgtcgccggagcggtcgagttctggaccgaccggct |
| | cgggttctcccgggacttcgtggaggacgacttcgccggtgtggtccgggacgac |
| | gtgaccctgttcatcagcgcggtccaggaccaggtggtgccggacaacaccctgg |
| | cctgggtgtgggtgcgcggcctggacgagctgtacgccgagtggtcggaggtcgt |
| | gtccacgaacttccgggacgcctccgggccggccatgaccgagatcggagagcag |
| | CCAtggGGACGTgagttcgccctgCGTgacccagccggaaactgcgtgcacttcg |
| | tggccGAAgaacaggactgacacgtgctacgagatttcgattccaccgccgcctt |
| | ctatgaaaggttgggcttcggaatcgttttccgggacgccggctggatgatcctc |
| | cagcgcggggatctcatgctggagttcttcgcccaccccaacttgtttattgcag |
| | cttataatggttacaaataaagcaatagcatcacaaatttcacaaataaagcatt |
| | tttttcactgcattctagttgtggtttgtccaaactcatcaatgtatcttatcat |
| | gtctgtataccgtcgacctctagctagagcttggcgtaatcatggtcatagctgt |
| | ttcctgtgtgaaattgttatccgctcacaattccacacaacatacgagccggaag |
| | cataaagtgtaaagcctggggtgcctaatgagtgagctaactcacattaattgcg |
| | ttgcgctcactgcccgctttccagtcgggaaacctgtcgtgccagctgcattaat |
| | gaatcggccaacgcgcggggagaggcggtttgcgtattgggcgctcttccgcttc |
| | ctcgctcactgactcgctgcgctcggtcgttcggctgcggcgagcggtatcagct |
| | cactcaaaggcggtaatacggttatccacagaatcaggggataacgcaggaaaga |
| | acatgtgagcaaaaggccagcaaaaggccaggaaccgtaaaaaggccgcgttgct |
| | ggcgtttttccataggctccgcccccctgacgagcatcacaaaaatcgacgctca |
| | agtcagaggtggcgaaacccgacaggactataaagataccaggcgtttccccctg |
| | gaagctccctcgtgcgctctcctgttccgaccctgccgcttaccggatacctgtc |
| | cgcctttctcccttcgggaagcgtggcgctttctcatagctcacgctgtaggtat |
| | ctcagttcggtgtaggtcgttcgctccaagctgggctgtgtgcacgaaccccccg |
| | ttcagcccgaccgctgcgccttatccggtaactatcgtcttgagtccaacccggt |
| | aagacacgacttatcgccactggcagcagccactggtaacaggattagcagagcg |
| | aggtatgtaggcggtgctacagagttcttgaagtggtggcctaactacggctaca |
| | ctagaagaacagtatttggtatctgcgctctgctgaagccagttaccttcggaaa |
| | aagagttggtagctcttgatccggcaaacaaaccaccgctggtagcggtggtttt |
| | tttgtttgcaagcagcagattacgcgcagaaaaaaaggatctcaagaagatcctt |
| | tgatcttttctacggggtctgacgctcagtggaacgaaaactcacgttaagggat |
| | tttggtcatgagattatcaaaaaggatcttcacctagatccttttaaattaaaaa |
| | tgaagttttaaatcaatctaaagtatatatgagtaaacttggtctgacagttacc |
| | aatgcttaatcagtgaggcacctatctcagcgatctgtctatttcgttcatccat |
| | agttgcctgactccccgtcgtgtagataactacgatacgggagggcttaccatct |
| | ggccccagtgctgcaatgataccgcgagacccacgctcaccggctccagatttat |
| | cagcaataaaccagccagccggaagggccgagcgcagaagtggtcctgcaacttt |
| | atccgcctccatccagtctattaattgttgccgggaagctagagtaagtagttcg |
| | ccagttaatagtttgcgcaacgttgttgccattgctacaggcatcgtggtgtcac |
| | gctcgtcgtttggtatggcttcattcagctccggttcccaacgatcaaggcgagt |
| | tacatgatcccccatgttgtgcaaaaaagcggttagctccttcggtcctccgatc |
| | gttgtcagaagtaagttggccgcagtgttatcactcatggttatggcagcactgc |
| | ataattctcttactgtcatgccatccgtaagatgcttttctgtgactggtgagta |
| | ctcaaccaagtcattctgagaatagtgtatgcggcgaccgagttgctcttgcccg |
| | gcgtcaatacgggataataccgcgccacatagcagaactttaaaagtgctcatca |
| | ttggaaaacgttcttcggggcgaaaactctcaaggatcttaccgctgttgagatc |
| | cagttcgatgtaacccactcgtgcacccaactgatcttcagcatcttttactttc |
| | accagcgtttctgggtgagcaaaaacaggaaggcaaaatgccgcaaaaaagggaa |
| | taagggcgacacggaaatgttgaatactcatactcttcctttttcaatattattg |
| | aagcatttatcagggttattgtctcatgagcggatacatatttgaatgtatttag |
| | aaaaataaacaaataggggttccgcgcacatttccccgaaaagtgccacctgacg |
| | tcgacggatcgggagatctcccgatcccctatggtgcactctcagtacaatctgc |
| | tctgatgccgcatagttaagccagtatctgctccctgcttgtgtgttggaggtcg |
| | ctgagtagtgcgcgagcaaaatttaagctacaacaaggcaaggcttgaccgacaa |
| | ttgcatgaagaatctgcttagggttaggcgttttgcgctgcttcgcgatgtacgg |
| | gccagatatacgcgttgacattgattattgactagttattaatagtaatcaatta |
| | cggggtcattagttcatagcccatatatggagttccgcgttacataacttacggt |
| | aaatggcccgcctggctgaccgcccaacgacccccgcccattgacgtcaataatg |
| | acgtatgttcccatagtaacgccaatagggactttccattgacgtcaatgggtgg |
| | agtatttacggtaaactgcccacttggcagtacatcaagtgtatcatatgccaag |
| | tacgccccctattgacgtcaatgacggtaaatggcccgcctggcattatgcccag |
| | tacatgaccttatgggactttcctacttggcagtacatctacgtattagtcatcg |
| | ctattaccatggtgatgcggttttggcagtacatcaatgggcgtggatagcggtt |
| | tgactcacggggatttccaagtctccaccccattgacgtcaatgggagtttgttt |
| | tggcaccaaaatcaacgggactttccaaaatgtcgtaacaactccgccccattga |
| | cgcaaatgggggtaggcgtgtacggtgggaggtctatataagcagcgcgttttgc |
| | ctgtactgggtctctctggttagaccagatctgagcctgggagctctctggctaa |
| | ctagggaacccactgcttaagcctcaataaagcttgccttgagtgcttcaagtag |
| | tgtgtgcccgtctgttgtgtgactctggtaactagagatccctcagaccctttta |
| | gtcagtgtggaaaatctctagcagtggcgcccgaacagggacttgaaagcgaaag |
| | ggaaaccagaggagctctctcgacgcaggactcggcttgctgaagcgcgcacggc |
| | aagaggcgaggggcggcgactggtgagtacgccaaaaattttgactagcggaggc |
| | tagaaggagagagatgggtgcgagagcgtcagtattaagcgggggagaattagat |
| | cgcgatgggaaaaaattcggttaaggccagggggaaagaaaaaatataaattaaa |
| | acatatagtatgggcaagcagggagctagaacgattcgcagttaatcctggcctg |
| | ttagaaacatcagaaggctgtagacaaatactgggacagctacaaccatcccttc |
| | agacaggatcagaagaacttagatcattatataatacagtagcaaccctctattg |
| | tgtgcatcaaaggatagagataaaagacaccaaggaagctttagacaagatagag |
| | gaagagcaaaacaaaagtaagaccaccgcacagcaagcggccgctgatcttcaga |
| | cctggaggaggagatatgagggacaattggagaagtgaattatataaatataaag |
| | tagtaaaaattgaaccattaggagtagcacccaccaaggcaaagagaagagtggt |
| | gcagagagaaaaaagagcagtgggaataggagctttgttccttgggttcttggga |
| | gcagcaggaagcactatgggcgcagcgtcaatgacgctgacggtacaggccagac |
| | aattattgtctggtatagtgcagcagcagaacaatttgctgagggctattgaggc |
| | gcaacagcatctgttgcaactcacagtctggggcatcaagcagctccaggcaaga |
| | atcctggctgtggaaagatacctaaaggatcaacagctcctggggatttggggtt |
| | gctctggaaaactcatttgcaccactgctgtgccttggaatgctagttggagtaa |
| | taaatctctggaacagatttggaatcacacgacctggatggagtgggacagagaa |
| | attaacaattacacaagcttaatacactccttaattgaagaatcgcaaaaccagc |
| | aagaaaagaatgaacaagaattattggaattagataaatgggcaagtttgtggaa |
| | ttggtttaacataacaaattggctgtggtatataaaattattcataatgatagta |
| | ggaggcttggtaggtttaagaatagtttttgctgtactttctatagtgaatagag |
| | ttaggcagggatattcaccattatcgtttcagacccacctcccaaccccgagggg |
| | acccgacaggcccgaaggaatagaagaagaaggtggagagagagacagagacaga |
| | tccattcgattagtgaacggatcggcactgcgtgcgccaattctgcagacaaatg |
| | gcagtattcatccacaattttaaaagaaaaggggggattggggggtacagtgcag |
| | gggaaagaatagtagacataatagcaacagacatacaaactaaagaattacaaaa |
| | acaaattacaaaaattcaaaattttcgggtttattacagggacagcagagatcca |
| | gtttggttaattaaggtaccGGAGtGAGACCGGAGatGTCTTCCGTTgctagccc |
| | tgcacgCTTCtttacagctagctcagtcctaggtattatgctagctactCGACct |
| | agtgAAAGAGGAGAAAtactaAATGgtgagtaaaggagaagaaaacaacttagct |
| | atcattaaagagttcatgcgcttcaaagttcacatggagggttctgttaacggtc |
| | acgagttcgagatcgaaggcgaaggcgagggccgtccgtatgaaggcacccagac |
| | cgccaaactgaaagtgactaaaggcggcccgctgccttttgcgtgggacatcctg |
| | agcccgcaatttatgtacggttctaaagcgtatgttaaacacccagcggatatcc |
| | cggactatctgaagctgtcttttccggaaggtttcaactgggaacgcgtaatgaa |
| | ttttgaagatggtggtgtcgtgaccgtcactcaggactcctccctTcaggatggc |
| | gagttcatctataaagttaaactgcgtggtactaattttccatctgatggcccgg |
| | tgatgcagtgtaggacgatgggttgggaggcgtctaccgaacgcatgtatccgga |
| | agatggtgcgctgaaaggcgaaattaaacagcgcctgaaactgaaagatggcggc |
| | cattatgacgctgaagtgaaaaccacgtacaaagccaagaaacctgtgcagctgc |
| | ctggcgcgtacaatgtggatattaaactggacatcttatctcataatgaagatta |
| | tacgatcgtagagcaatatgagcgcgcggagggtcgtcattctaccggtggcatg |
| | gatgaactatacaaataaTGGCctcgcgtacgcaaagacgaacaataagacgctg |
| | aaaagcgtcttttttcgttttggtccGCTGTTCCtGAAGACatTCGCtaagtcga |
| | caatcaacctctggattacaaaatttgtgaaagattgactggtattcttaactat |
| | gttgctccttttacgctatgtggatacgctgctttaatgcctttgtatcatgcta |
| | ttgcttcccgtatggctttcattttctcctccttgtataaatcctggttgctgtc |
| | tctttatgaggagttgtggcccgttgtcaggcaacgtggcgtggtgtgcactgtg |
| | tttgctgacgcaacccccactggttggggcattgccaccacctgtcagctccttt |
| | ccgggactttcgctttccccctccctattgccacggcggaactcatcgccgcctg |
| | ccttgcccgctgctggacaggggctcggctgttgggcactgacaattccgtggtg |
| | ttgtcggggaaatcatcgtcctttccttggctgctcgcctgtgttgccacctgga |
| | ttctgcgcgggacgtccttctgctacgtcccttcggccctcaatccagcggacct |
| | tccttcccgcggcctgctgccggctctgcggcctcttccgcgtctAcgccttcgc |
| | cctcagacgagtcggatctccctttgggccgcctccccgcgtcgactttaagacc |
| | aatgacttacaaggcagctgtagatcttagccactttttaaaagaaaagggggga |
| | ctggaagggctaattcactcccaacgaagTcaagatctgctttttgcttgtactg |
| | ggtctctctggttagaccagatctgagcctgggagctctctggctaactagggaa |
| | cccactgcttaagcctcaataaagcttgccttgagtgcttcaagtagtgtgtgcc |
| | cgtctgttgtgtgactctggtaactagagatccctcagacccttttagtcagtgt |
| | ggaaaatctctagcagggcccgtttaaacccgctgatcagcctcgactgtgcctt |
| | ctagttgccagccatctgttgtttgcccctcccccgtgccttccttgaccctgga |
| | aggtgccactcccactgtcctttcctaataaaatgaggaaattgcatcgcattgt |
| | ctgagtaggtgtcattctattctggggggtggggtggggcaggacagcaaggggg |
| | aggattgggaagagaa |
| | (SEQ ID NO: 33) |
| pAJW_R1 | ataaaagacaccaaggaagctttagacaagatagaggaagagcaaaacaaaagta |
| | agaccaccgcacagcaagcggccgctgatcttcagacctggaggaggagatatga |
| | gggacaattggagaagtgaattatataaatataaagtagtaaaaattgaaccatt |
| | aggagtagcacccaccaaggcaaagagaagagtggtgcagagagaaaaaagagca |
| | gtgggaataggagctttgttccttgggttcttgggagcagcaggaagcactatgg |
| | gcgcagcgtcaatgacgctgacggtacaggccagacaattattgtctggtatagt |
| | gcagcagcagaacaatttgctgagggctattgaggcgcaacagcatctgttgcaa |
| | ctcacagtctggggcatcaagcagctccaggcaagaatcctggctgtggaaagat |
| | acctaaaggatcaacagctcctggggatttggggttgctctggaaaactcatttg |
| | caccactgctgtgccttggaatgctagttggagtaataaatctctggaacagatt |
| | tggaatcacacgacctggatggagtgggacagagaaattaacaattacacaagct |
| | taatacactccttaattgaagaatcgcaaaaccagcaagaaaagaatgaacaaga |
| | attattggaattagataaatgggcaagtttgtggaattggtttaacataacaaat |
| | tggctgtggtatataaaattattcataatgatagtaggaggcttggtaggtttaa |
| | gaatagtttttgctgtactttctatagtgaatagagttaggcagggatattcacc |
| | attatcgtttcagacccacctcccaaccccgaggggacccgacaggcccgaagga |
| | atagaagaagaaggtggagagagagacagagacagatccattcgattagtgaacg |
| | gatcggcactgcgtgcgccaattctgcagacaaatggcagtattcatccacaatt |
| | ttaaaagaaaaggggggattggggggtacagtgcaggggaaagaatagtagacat |
| | aatagcaacagacatacaaactaaagaattacaaaaacaaattacaaaaattcaa |
| | aattttcgggtttattacagggacagcagagatccagtttggttaattaaggtAC |
| | CGGAGCGTTttacttgtacagctcgtccatgccgagagtgatcccggcggcggtc |
| | acgaactccagcaggaccatgtgatcgcgcttctcgttggggtctttgctcaggg |
| | cggactgggtgctcaggtagtggttgtcgggcagcagcacggggccgtcgccgat |
| | gggggtgttctgctggtagtggtcggcgagctgcacgctgccgtcctcgatgttg |
| | tggcggatcttgaagttcaccttgatgccgttcttctgcttgtcggccatgatat |
| | agacgttgtggctgttgtagttgtactccagcttgtgccccaggatgttgccgtc |
| | ctccttgaagtcgatgcccttcagctcgatgcggttcaccagggtgtcgccctcg |
| | aacttcacctcggcgcgggtcttgtagttgccgtcgtccttgaagaagatggtgc |
| | gctcctggacgtagccttcgggcatggcggacttgaagaagtcgtgctgcttcat |
| | gtggtcggggtagcggctgaagcactgcacgccgtaggtcagggtggtcacgagg |
| | gtgggccagggcacgggcagcttgccggtggtgcagatgaacttcagggtcagct |
| | tgccgtaggtggcatcgccctcgccctcgccggacacgctgaacttgtggccgtt |
| | tacgtcgccgtccagctcgaccaggatgggcaccaccccggtgaacagctcctcg |
| | cccttgctcacCATAGCCATGGTGGCGGCATTCgcttggttctccctatagtgag |
| | tcgtattaatttcgataagccagtaagcagtgggttctctagttagccagagagc |
| | tctgcttatatagacctcccaccgtacacgcctaccgcccatttgcgtcaatggg |
| | gcggagttgttacgacattttggaaagtcccgttgattttggtgccaaaacaaac |
| | tcccattgacgtcaatggggtggagacttggaaatccccgtgagtcaaaccgcta |
| | tccacgcccattgatgtactgccaaaaccgcatcaccatggtaatagcgatgact |
| | aatacgtagatgtactgccaagtaggaaagtcccataaggtcatgtactgggcat |
| | aatgccaggcgggccatttaccgtcattgacgtcaatagggggcgtacttggcat |
| | atgatacacttgatgtactgccaagtgggcagtttaccgtaaatactccacccat |
| | tgacgtcaatggaaagtccctattggcgttactatgggaacatacgtcattattg |
| | acgtcaatgggcgggggtcgttgggcggtcagccaggcgggccatttaccgtaag |
| | ttatgtaacgcggaactccatatatgggctatgaactaatgaccccgtaattgat |
| | tactattaataactagtcaataatcaatgtcaacgagtatatctggTTTGagtgg |
| | ttttggggtttttccataggatattggaactggtggaattttgggagttaaccag |
| | cttaaccccagttcaaaggtatgaagtgacttcttcaaaacaaaccaaaatgaac |
| | acactttcccaccaaagaatattagtttagaattgaacgtttgttcaccctgtgt |
| | aaaccaaTGCTGCCCCCTAGTGGCGAaggtggcaagttcaagcaagtcaccttga |
| | gggccaggaaattagtaattaggctaatataatttcccaaagagcattgcaaaag |
| | aGCGAGGCATCGCCAAAatcaactttgtatagaaaagttgctcgacattgattat |
| | tgactagttattaatagtaatcaattacggggtcattagttcatagcccatatat |
| | ggagttccgcgttacataacttacggtaaatggcccgcctggctgaccgcccaac |
| | gacccccgcccattgacgtcaataatgacgtatgttcccatagtaacgccaatag |
| | ggactttccattgacgtcaatgggtggagtatttacggtaaactgcccacttggc |
| | agtacatcaagtgtatcatatgccaagtacgccccctattgacgtcaatgacggt |
| | aaatggcccgcctggcattatgcccagtacatgaccttatgggactttcctactt |
| | ggcagtacatctacgtattagtcatcgctattaccatggtcgaggtgagccccac |
| | gttctgcttcactctccccatctcccccccctccccacccccaattttgtattta |
| | tttattttttaattattttgtgcagcgatgggggcggggggggggggggggcgcg |
| | cgccaggcggggggggggggcgaggggggggcggggcgaggcggagaggtgcggc |
| | ggcagccaatcagagcggcgcgctccgaaagtttccttttatggcgaggcggcgg |
| | cggcggcggccctataaaaagcgaagcgcgcggggggggagtcgctgcgcgctgc |
| | cttcgccccgtgccccgctccgccgccgcctcgcgccgcccgccccggctctgac |
| | tgaccgcgttactcccacaggtgagcggggggacggcccttctcctccgggctgt |
| | aattagcgcttggtttaatgacggcttgtttcttttctgtggctgcgtgaaagcc |
| | ttgaggggctccgggagggccctttgtgcggggggagcggctcggggggtgcgtg |
| | cgtgtgtgtgtgcgtggggagcgccgcgtgcggctccgcgctgcccggcggctgt |
| | gagcgctgcgggcgcggcgcggggctttgtgcgctccgcagtgtgcgcgagggga |
| | gcgcggccgggggcggtgccccgcggtgcggggggggctgcgaggggaacaaagg |
| | ctgcgtgcggggtgtgtgcgtggggggggagcagggggtgtgggcgcgtcggtcg |
| | ggctgcaaccccccctgcacccccctccccgagttgctgagcacggcccggcttc |
| | gggtgcggggctccgtacggggcgtggcgcggggctcgccgtgccgggcgggggg |
| | ggcggcaggtgggggtgccgggcggggggggccgcctcgggccggggagggctcg |
| | ggggaggggcgcggcggcccccggagcgccggcggctgtcgaggcgcggcgagcc |
| | gcagccattgccttttatggtaatcgtgcgagagggcgcagggacttcctttgtc |
| | ccaaatctgtgcggagccgaaatctgggaggcgccgccgcaccccctctagcggg |
| | cgcggggcgaagcggtgcggcgccggcaggaaggaaatgggcggggagggccttc |
| | gtgcgtcgccgcgccgccgtccccttctccctctccagcctcggggctgtccgcg |
| | gggggacggctgccttcgggggggacggggcagggcggggttcggcttctggcgt |
| | gtgaccggcggctctagagcctctgctaaccatgttcatgccttcttctttttcc |
| | tacagctcctgggcaacgtgctggttattgtgctgtctcatcattttggcaaaga |
| | attgcaagtttgtacaaaaaagcaggctGAATGCCGCCACCATGGCTaccgagta |
| | taaaccgacggtccgacttgcgactcgggatgacgttccacgcgccgtgaggact |
| | ctggccgccgccttcgctgactacccggccacacggcacacagttgaccccgaca |
| | gacacatagagcgggtcacagagttgcaagaacttttcctgacgcgggttggAct |
| | cgatattggaaaagtttgggttgccgacgatggtgccgcagttgcggtgtggact |
| | actccagaaagtgtagaagctggagccgtattcgctgaaatagggccacggatgg |
| | cagagctgtctggtagcaggctcgcagcgcagcagcagatggaaggCcttctcgc |
| | tccgcatcggcctaaggaaccggcgtggttcctggctacggtgggagtctcacct |
| | gaccatcaaggaaagggattgggaagtgctgtcgttcttccaggcgtggaagcgg |
| | ccgaacgagcaggcgtcccagccttcttggaAacgagcgcacccaggaatcttcc |
| | attctacgagcgactgggtttcactgtgacagcagacgtagaagtccctgaaggg |
| | cctcggacttggtgcatgaccagaaagcctggggcttgaAACGTCGcaatcaacc |
| | tctggattacaaaatttgtgaaagattgactggtattcttaactatgttgctcct |
| | tttacgctatgtggatacgctgctttaatgcctttgtatcatgctattgcttccc |
| | gtatggctttcattttctcctccttgtataaatcctggttgctgtctctttatga |
| | ggagttgtggcccgttgtcaggcaacgtggcgtggtgtgcactgtgtttgctgac |
| | gcaacccccactggttggggcattgccaccacctgtcagctcctttccgggactt |
| | tcgctttccccctccctattgccacggcggaactcatcgccgcctgccttgcccg |
| | ctgctggacaggggctcggctgttgggcactgacaattccgtggtgttgtcgggg |
| | aaatcatcgtcctttccttggctgctcgcctgtgttgccacctggattctgcgcg |
| | ggacgtccttctgctacgtcccttcggccctcaatccagcggaccttccttcccg |
| | cggcctgctgccggctctgcggcctcttccgcgtctAcgccttcgccctcagacg |
| | agtcggatctccctttgggccgcctccccgcgtcgactttaagaccaatgactta |
| | caaggcagctgtagatcttagccactttttaaaagaaaaggggggactggaaggg |
| | ctaattcactcccaacgaagTcaagatctgctttttgcttgtactgggtctctct |
| | ggttagaccagatctgagcctgggagctctctggctaactagggaacccactgct |
| | taagcctcaataaagcttgccttgagtgcttcaagtagtgtgtgcccgtctgttg |
| | tgtgactctggtaactagagatccctcagacccttttagtcagtgtggaaaatct |
| | ctagcagggcccgtttaaacccgctgatcagcctcgactgtgccttctagttgcc |
| | agccatctgttgtttgcccctcccccgtgccttccttgaccctggaaggtgccac |
| | tcccactgtcctttcctaataaaatgaggaaattgcatcgcattgtctgagtagg |
| | tgtcattctattctggggggtggggtggggcaggacagcaagggggaggattggg |
| | aagagaatagcaggcatgctggggatgcggtgggctctatggcttctgaggcgga |
| | aagaaccagctggggctctagggggtatccccacgcgccctgtagcggcgcatta |
| | agcgcggcgggtgtggtggttacgcgcagcgtgaccgctacacttgccagcgccc |
| | tagcgcccgctcctttcgctttcttcccttcctttctcgccacgttcgccggctt |
| | tccccgtcaagctctaaatcgggggctccctttagggttccgatttagtgcttta |
| | cggcacctcgaccccaaaaaacttgattagggtgatggttcacgtagtgggccat |
| | cgccctgatagacggtttttcgccctttgacgttggagtccacgttctttaatag |
| | tggactcttgttccaaactggaacaacactcaaccctatctcggtctattctttt |
| | gatttataagggattttgccgatttcggcctattggttaaaaaatgagctgattt |
| | aacaaaaatttaacgcgaattaattctgtggaatgtgtgtcagttagggtgtgga |
| | aagtccccaggctccccagcaggcagaagtatgcaaagcatgcatctcaattagt |
| | cagcaaccaggtgtggaaagtccccaggctccccagcaggcagaagtatgcaaag |
| | catgcatctcaattagtcagcaaccatagtcccgcccctaactccgcccatcccg |
| | cccctaactccgcccagttccgcccattctccgccccatggctgactaatttttt |
| | ttatttatgcagaggccgaggccgcctctgcctctgagctattccagaagtagtg |
| | aggaggcttttttggaggcctaggcttttgcaaaaagctcccgggagcttgtata |
| | tccattttcggatctgatcagcacgtgttgacaattaatcatcggcatagtatat |
| | cggcatagtataatacgacaaggtgaggaactaaaccatggccaagttgaccagt |
| | gccgttccggtgctcaccgcgcgcgacgtcgccggagcggtcgagttctggaccg |
| | accggctcgggttctcccgggacttcgtggaggacgacttcgccggtgtggtccg |
| | ggacgacgtgaccctgttcatcagcgcggtccaggaccaggtggtgccggacaac |
| | accctggcctgggtgtgggtgcgcggcctggacgagctgtacgccgagtggtcgg |
| | aggtcgtgtccacgaacttccgggacgcctccgggccggccatgaccgagatcgg |
| | cgagcagccgtggggggggagttcgccctgcgcgacccggccggcaactgcgtgc |
| | acttcgtggccgaggagcaggactgacacgtgctacgagatttcgattccaccgc |
| | cgccttctatgaaaggttgggcttcggaatcgttttccgggacgccggctggatg |
| | atcctccagcgcggggatctcatgctggagttcttcgcccaccccaacttgttta |
| | ttgcagcttataatggttacaaataaagcaatagcatcacaaatttcacaaataa |
| | agcatttttttcactgcattctagttgtggtttgtccaaactcatcaatgtatct |
| | tatcatgtctgtataccgtcgacctctagctagagcttggcgtaatcatggtcat |
| | agctgtttcctgtgtgaaattgttatccgctcacaattccacacaacatacgagc |
| | cggaagcataaagtgtaaagcctggggtgcctaatgagtgagctaactcacatta |
| | attgcgttgcgctcactgcccgctttccagtcgggaaacctgtcgtgccagctgc |
| | attaatgaatcggccaacgcgcggggagaggcggtttgcgtattgggcgctcttc |
| | cgcttcctcgctcactgactcgctgcgctcggtcgttcggctgcggcgagcggta |
| | tcagctcactcaaaggcggtaatacggttatccacagaatcaggggataacgcag |
| | gaaagaacatgtgagcaaaaggccagcaaaaggccaggaaccgtaaaaaggccgc |
| | gttgctggcgtttttccataggctccgcccccctgacgagcatcacaaaaatcga |
| | cgctcaagtcagaggtggcgaaacccgacaggactataaagataccaggcgtttc |
| | cccctggaagctccctcgtgcgctctcctgttccgaccctgccgcttaccggata |
| | cctgtccgcctttctcccttcgggaagcgtggcgctttctcatagctcacgctgt |
| | aggtatctcagttcggtgtaggtcgttcgctccaagctgggctgtgtgcacgaac |
| | cccccgttcagcccgaccgctgcgccttatccggtaactatcgtcttgagtccaa |
| | cccggtaagacacgacttatcgccactggcagcagccactggtaacaggattagc |
| | agagcgaggtatgtaggcggtgctacagagttcttgaagtggtggcctaactacg |
| | gctacactagaagaacagtatttggtatctgcgctctgctgaagccagttacctt |
| | cggaaaaagagttggtagctcttgatccggcaaacaaaccaccgctggtagcggt |
| | ggtttttttgtttgcaagcagcagattacgcgcagaaaaaaaggatctcaagaag |
| | atcctttgatcttttctacggggtctgacgctcagtggaacgaaaactcacgtta |
| | agggattttggtcatgagattatcaaaaaggatcttcacctagatccttttaaat |
| | taaaaatgaagttttaaatcaatctaaagtatatatgagtaaacttggtctgaca |
| | gttaccaatgcttaatcagtgaggcacctatctcagcgatctgtctatttcgttc |
| | atccatagttgcctgactccccgtcgtgtagataactacgatacgggagggctta |
| | ccatctggccccagtgctgcaatgataccgcgagacccacgctcaccggctccag |
| | atttatcagcaataaaccagccagccggaagggccgagcgcagaagtggtcctgc |
| | aactttatccgcctccatccagtctattaattgttgccgggaagctagagtaagt |
| | agttcgccagttaatagtttgcgcaacgttgttgccattgctacaggcatcgtgg |
| | tgtcacgctcgtcgtttggtatggcttcattcagctccggttcccaacgatcaag |
| | gcgagttacatgatcccccatgttgtgcaaaaaagcggttagctccttcggtcct |
| | ccgatcgttgtcagaagtaagttggccgcagtgttatcactcatggttatggcag |
| | cactgcataattctcttactgtcatgccatccgtaagatgcttttctgtgactgg |
| | tgagtactcaaccaagtcattctgagaatagtgtatgcggcgaccgagttgctct |
| | tgcccggcgtcaatacgggataataccgcgccacatagcagaactttaaaagtgc |
| | tcatcattggaaaacgttcttcggggcgaaaactctcaaggatcttaccgctgtt |
| | gagatccagttcgatgtaacccactcgtgcacccaactgatcttcagcatctttt |
| | actttcaccagcgtttctgggtgagcaaaaacaggaaggcaaaatgccgcaaaaa |
| | agggaataagggcgacacggaaatgttgaatactcatactcttcctttttcaata |
| | ttattgaagcatttatcagggttattgtctcatgagcggatacatatttgaatgt |
| | atttagaaaaataaacaaataggggttccgcgcacatttccccgaaaagtgccac |
| | ctgacgtcgacggatcgggagatctcccgatcccctatggtgcactctcagtaca |
| | atctgctctgatgccgcatagttaagccagtatctgctccctgcttgtgtgttgg |
| | aggtcgctgagtagtgcgcgagcaaaatttaagctacaacaaggcaaggcttgac |
| | cgacaattgcatgaagaatctgcttagggttaggcgttttgcgctgcttcgcgat |
| | gtacgggccagatatacgcgttgacattgattattgactagttattaatagtaat |
| | caattacggggtcattagttcatagcccatatatggagttccgcgttacataact |
| | tacggtaaatggcccgcctggctgaccgcccaacgacccccgcccattgacgtca |
| | ataatgacgtatgttcccatagtaacgccaatagggactttccattgacgtcaat |
| | gggtggagtatttacggtaaactgcccacttggcagtacatcaagtgtatcatat |
| | gccaagtacgccccctattgacgtcaatgacggtaaatggcccgcctggcattat |
| | gcccagtacatgaccttatgggactttcctacttggcagtacatctacgtattag |
| | tcatcgctattaccatggtgatgcggttttggcagtacatcaatgggcgtggata |
| | gcggtttgactcacggggatttccaagtctccaccccattgacgtcaatgggagt |
| | ttgttttggcaccaaaatcaacgggactttccaaaatgtcgtaacaactccgccc |
| | cattgacgcaaatgggcggtaggcgtgtacggtgggaggtctatataagcagcgc |
| | gttttgcctgtactgggtctctctggttagaccagatctgagcctgggagctctc |
| | tggctaactagggaacccactgcttaagcctcaataaagcttgccttgagtgctt |
| | caagtagtgtgtgcccgtctgttgtgtgactctggtaactagagatccctcagac |
| | ccttttagtcagtgtggaaaatctctagcagtggcgcccgaacagggacttgaaa |
| | gcgaaagggaaaccagaggagctctctcgacgcaggactcggcttgctgaagcgc |
| | gcacggcaagaggcgaggggcggcgactggtgagtacgccaaaaattttgactag |
| | cggaggctagaaggagagagatgggtgcgagagcgtcagtattaagcgggggaga |
| | attagatcgcgatgggaaaaaattcggttaaggccagggggaaagaaaaaatata |
| | aattaaaacatatagtatgggcaagcagggagctagaacgattcgcagttaatcc |
| | tggcctgttagaaacatcagaaggctgtagacaaatactgggacagctacaacca |
| | tcccttcagacaggatcagaagaacttagatcattatataatacagtagcaaccc |
| | tctattgtgtgcatcaaaggatagag |
| | (SEQ ID NO: 34) |
| pAJW_R2 | tagcaggcatgctggggatgcggtgggctctatggcttctgaggcggaaagaacc |
| | agctggggctctagggggtatccccacgcgccctgtagcggcgcattaagcgcgg |
| | cgggtgtggtggttacgcgcagcgtgaccgctacacttgccagcgccctagcgcc |
| | cgctcctttcgctttcttcccttcctttctcgccacgttcgccggctttccccgt |
| | caagctctaaatcgggggctccctttagggttccgatttagtgctttacggcacc |
| | tcgaccccaaaaaacttgattagggtgatggttcacgtagtgggccatcgccctg |
| | atagacggtttttcgccctttgacgttggagtccacgttctttaatagtggactc |
| | ttgttccaaactggaacaacactcaaccctatctcggtctattcttttgatttat |
| | aagggattttgccgatttcggcctattggttaaaaaatgagctgatttaacaaaa |
| | atttaacgcgaattaattctgtggaatgtgtgtcagttagggtgtggaaagtccc |
| | caggctccccagcaggcagaagtatgcaaagcatgcatctcaattagtcagcaac |
| | caggtgtggaaagtccccaggctccccagcaggcagaagtatgcaaagcatgcat |
| | ctcaattagtcagcaaccatagtcccgcccctaactccgcccatcccgcccctaa |
| | ctccgcccagttccgcccattctccgccccatggctgactaattttttttattta |
| | tgcagaggccgaggccgcctctgcctctgagctattccagaagtagtgaggaggc |
| | ttttttggaggcctaggcttttgcaaaaagctcccgggagcttgtatatccattt |
| | tcggatctgatcagcacgtgttgacaattaatcatcggcatagtatatcggcata |
| | gtataatacgacaaggtgaggaactaaaccatggccaagttgaccagtgccgttc |
| | cggtgctcaccgcgcgcgacgtcgccggagcggtcgagttctggaccgaccggct |
| | cgggttctcccgggacttcgtggaggacgacttcgccggtgtggtccgggacgac |
| | gtgaccctgttcatcagcgcggtccaggaccaggtggtgccggacaacaccctgg |
| | cctgggtgtgggtgcgcggcctggacgagctgtacgccgagtggtcggaggtcgt |
| | gtccacgaacttccgggacgcctccgggccggccatgaccgagatcggcgagcag |
| | ccgtgggggcgggagttcgccctgcgcgacccggccggcaactgcgtgcacttcg |
| | tggccgaggagcaggactgacacgtgctacgagatttcgattccaccgccgcctt |
| | ctatgaaaggttgggcttcggaatcgttttccgggacgccggctggatgatcctc |
| | cagcgcggggatctcatgctggagttcttcgcccaccccaacttgtttattgcag |
| | cttataatggttacaaataaagcaatagcatcacaaatttcacaaataaagcatt |
| | tttttcactgcattctagttgtggtttgtccaaactcatcaatgtatcttatcat |
| | gtctgtataccgtcgacctctagctagagcttggcgtaatcatggtcatagctgt |
| | ttcctgtgtgaaattgttatccgctcacaattccacacaacatacgagccggaag |
| | cataaagtgtaaagcctggggtgcctaatgagtgagctaactcacattaattgcg |
| | ttgcgctcactgcccgctttccagtcgggaaacctgtcgtgccagctgcattaat |
| | gaatcggccaacgcgcggggagaggcggtttgcgtattgggcgctcttccgcttc |
| | ctcgctcactgactcgctgcgctcggtcgttcggctgcggcgagcggtatcagct |
| | cactcaaaggcggtaatacggttatccacagaatcaggggataacgcaggaaaga |
| | acatgtgagcaaaaggccagcaaaaggccaggaaccgtaaaaaggccgcgttgct |
| | ggcgtttttccataggctccgcccccctgacgagcatcacaaaaatcgacgctca |
| | agtcagaggtggcgaaacccgacaggactataaagataccaggcgtttccccctg |
| | gaagctccctcgtgcgctctcctgttccgaccctgccgcttaccggatacctgtc |
| | cgcctttctcccttcgggaagcgtggcgctttctcatagctcacgctgtaggtat |
| | ctcagttcggtgtaggtcgttcgctccaagctgggctgtgtgcacgaaccccccg |
| | ttcagcccgaccgctgcgccttatccggtaactatcgtcttgagtccaacccggt |
| | aagacacgacttatcgccactggcagcagccactggtaacaggattagcagagcg |
| | aggtatgtaggcggtgctacagagttcttgaagtggtggcctaactacggctaca |
| | ctagaagaacagtatttggtatctgcgctctgctgaagccagttaccttcggaaa |
| | aagagttggtagctcttgatccggcaaacaaaccaccgctggtagcggtggtttt |
| | tttgtttgcaagcagcagattacgcgcagaaaaaaaggatctcaagaagatcctt |
| | tgatcttttctacggggtctgacgctcagtggaacgaaaactcacgttaagggat |
| | tttggtcatgagattatcaaaaaggatcttcacctagatccttttaaattaaaaa |
| | tgaagttttaaatcaatctaaagtatatatgagtaaacttggtctgacagttacc |
| | aatgcttaatcagtgaggcacctatctcagcgatctgtctatttcgttcatccat |
| | agttgcctgactccccgtcgtgtagataactacgatacgggagggcttaccatct |
| | ggccccagtgctgcaatgataccgcgagacccacgctcaccggctccagatttat |
| | cagcaataaaccagccagccggaagggccgagcgcagaagtggtcctgcaacttt |
| | atccgcctccatccagtctattaattgttgccgggaagctagagtaagtagttcg |
| | ccagttaatagtttgcgcaacgttgttgccattgctacaggcatcgtggtgtcac |
| | gctcgtcgtttggtatggcttcattcagctccggttcccaacgatcaaggcgagt |
| | tacatgatcccccatgttgtgcaaaaaagcggttagctccttcggtcctccgatc |
| | gttgtcagaagtaagttggccgcagtgttatcactcatggttatggcagcactgc |
| | ataattctcttactgtcatgccatccgtaagatgcttttctgtgactggtgagta |
| | ctcaaccaagtcattctgagaatagtgtatgcggcgaccgagttgctcttgcccg |
| | gcgtcaatacgggataataccgcgccacatagcagaactttaaaagtgctcatca |
| | ttggaaaacgttcttcggggcgaaaactctcaaggatcttaccgctgttgagatc |
| | cagttcgatgtaacccactcgtgcacccaactgatcttcagcatcttttactttc |
| | accagcgtttctgggtgagcaaaaacaggaaggcaaaatgccgcaaaaaagggaa |
| | taagggcgacacggaaatgttgaatactcatactcttcctttttcaatattattg |
| | aagcatttatcagggttattgtctcatgagcggatacatatttgaatgtatttag |
| | aaaaataaacaaataggggttccgcgcacatttccccgaaaagtgccacctgacg |
| | tcgacggatcgggagatctcccgatcccctatggtgcactctcagtacaatctgc |
| | tctgatgccgcatagttaagccagtatctgctccctgcttgtgtgttggaggtcg |
| | ctgagtagtgcgcgagcaaaatttaagctacaacaaggcaaggcttgaccgacaa |
| | ttgcatgaagaatctgcttagggttaggcgttttgcgctgcttcgcgatgtacgg |
| | gccagatatacgcgttgacattgattattgactagttattaatagtaatcaatta |
| | cggggtcattagttcatagcccatatatggagttccgcgttacataacttacggt |
| | aaatggcccgcctggctgaccgcccaacgacccccgcccattgacgtcaataatg |
| | acgtatgttcccatagtaacgccaatagggactttccattgacgtcaatgggtgg |
| | agtatttacggtaaactgcccacttggcagtacatcaagtgtatcatatgccaag |
| | tacgccccctattgacgtcaatgacggtaaatggcccgcctggcattatgcccag |
| | tacatgaccttatgggactttcctacttggcagtacatctacgtattagtcatcg |
| | ctattaccatggtgatgcggttttggcagtacatcaatgggcgtggatagcggtt |
| | tgactcacggggatttccaagtctccaccccattgacgtcaatgggagtttgttt |
| | tggcaccaaaatcaacgggactttccaaaatgtcgtaacaactccgccccattga |
| | cgcaaatgggcggtaggcgtgtacggtgggaggtctatataagcagcgcgttttg |
| | cctgtactgggtctctctggttagaccagatctgagcctgggagctctctggcta |
| | actagggaacccactgcttaagcctcaataaagcttgccttgagtgcttcaagta |
| | gtgtgtgcccgtctgttgtgtgactctggtaactagagatccctcagaccctttt |
| | agtcagtgtggaaaatctctagcagtggcgcccgaacagggacttgaaagcgaaa |
| | gggaaaccagaggagctctctcgacgcaggactcggcttgctgaagcgcgcacgg |
| | caagaggcgaggggcggcgactggtgagtacgccaaaaattttgactagcggagg |
| | ctagaaggagagagatgggtgcgagagcgtcagtattaagcgggggagaattaga |
| | tcgcgatgggaaaaaattcggttaaggccagggggaaagaaaaaatataaattaa |
| | aacatatagtatgggcaagcagggagctagaacgattcgcagttaatcctggcct |
| | gttagaaacatcagaaggctgtagacaaatactgggacagctacaaccatccctt |
| | cagacaggatcagaagaacttagatcattatataatacagtagcaaccctctatt |
| | gtgtgcatcaaaggatagagataaaagacaccaaggaagctttagacaagataga |
| | ggaagagcaaaacaaaagtaagaccaccgcacagcaagcggccgctgatcttcag |
| | acctggaggaggagatatgagggacaattggagaagtgaattatataaatataaa |
| | gtagtaaaaattgaaccattaggagtagcacccaccaaggcaaagagaagagtgg |
| | tgcagagagaaaaaagagcagtgggaataggagctttgttccttgggttcttggg |
| | agcagcaggaagcactatgggcgcagcgtcaatgacgctgacggtacaggccaga |
| | caattattgtctggtatagtgcagcagcagaacaatttgctgagggctattgagg |
| | cgcaacagcatctgttgcaactcacagtctggggcatcaagcagctccaggcaag |
| | aatcctggctgtggaaagatacctaaaggatcaacagctcctggggatttggggt |
| | tgctctggaaaactcatttgcaccactgctgtgccttggaatgctagttggagta |
| | ataaatctctggaacagatttggaatcacacgacctggatggagtgggacagaga |
| | aattaacaattacacaagcttaatacactccttaattgaagaatcgcaaaaccag |
| | caagaaaagaatgaacaagaattattggaattagataaatgggcaagtttgtgga |
| | attggtttaacataacaaattggctgtggtatataaaattattcataatgatagt |
| | aggaggcttggtaggtttaagaatagtttttgctgtactttctatagtgaataga |
| | gttaggcagggatattcaccattatcgtttcagacccacctcccaaccccgaggg |
| | gacccgacaggcccgaaggaatagaagaagaaggtggagagagagacagagacag |
| | atccattcgattagtgaacggatcggcactgcgtgcgccaattctgcagacaaat |
| | ggcagtattcatccacaattttaaaagaaaaggggggattggggggtacagtgca |
| | ggggaaagaatagtagacataatagcaacagacatacaaactaaagaattacaaa |
| | aacaaattacaaaaattcaaaattttcgggtttattacagggacagcagagatcc |
| | agtttggttaattaaggtaccGGAGtGAGACCGGAGCGTTttacttgtacagctc |
| | gtccatgccgagagtgatcccggcggcggtcacgaactccagcaggaccatgtga |
| | tcgcgcttctcgttggggtctttgctcagggcggactgggtgctcaggtagtggt |
| | tgtcgggcagcagcacggggccgtcgccgatgggggtgttctgctggtagtggtc |
| | ggcgagctgcacgctgccgtcctcgatgttgtggcggatcttgaagttcaccttg |
| | atgccgttcttctgcttgtcggccatgatatagacgttgtggctgttgtagttgt |
| | actccagcttgtgccccaggatgttgccgtcctccttgaagtcgatgcccttcag |
| | ctcgatgcggttcaccagggtgtcgccctcgaacttcacctcggcgcgggtcttg |
| | tagttgccgtcgtccttgaagaagatggtgcgctcctggacgtagccttcgggca |
| | tggcggacttgaagaagtcgtgctgcttcatgtggtcggggtagcggctgaagca |
| | ctgcacgccgtaggtcagggtggtcacgagggtgggccagggcacgggcagcttg |
| | ccggtggtgcagatgaacttcagggtcagcttgccgtaggtggcatcgccctcgc |
| | cctcgccggacacgctgaacttgtggccgtttacgtcgccgtccagctcgaccag |
| | gatgggcaccaccccggtgaacagctcctcgcccttgctcacAGCCATGGTGGCG |
| | GCATTCgcttggttctccctatagtgagtcgtattaatttcgataagccagtaag |
| | cagtgggttctctagttagccagagagctctgcttatatagacctcccaccgtac |
| | acgcctaccgcccatttgcgtcaatggggcggagttgttacgacattttggaaag |
| | tcccgttgattttggtgccaaaacaaactcccattgacgtcaatggggtggagac |
| | ttggaaatccccgtgagtcaaaccgctatccacgcccattgatgtactgccaaaa |
| | ccgcatcaccatggtaatagcgatgactaatacgtagatgtactgccaagtagga |
| | aagtcccataaggtcatgtactgggcataatgccaggcgggccatttaccgtcat |
| | tgacgtcaatagggggcgtacttggcatatgatacacttgatgtactgccaagtg |
| | ggcagtttaccgtaaatactccacccattgacgtcaatggaaagtccctattggc |
| | gttactatgggaacatacgtcattattgacgtcaatgggcgggggtcgttgggcg |
| | gtcagccaggcgggccatttaccgtaagttatgtaacgcggaactccatatatgg |
| | gctatgaactaatgaccccgtaattgattactattaataactagtcaataatcaa |
| | tgtcaacgagtatatctggTTTGagtggttttggggtttttccataggatattgg |
| | aactggtggaattttgggagttaaccagcttaaccccagttcaaaggtatgaagt |
| | gacttcttcaaaacaaaccaaaatgaacacactttcccaccaaagaatattagtt |
| | tagaattgaacgtttgttcaccctgtgtaaaccaaTGCTGCCCCCTAGTGGCGAa |
| | ggtggcaagttcaagcaagtcaccttgagggccaggaaattagtaattaggctaa |
| | tataatttcccaaagagcattgcaaaagaGCGAGGCAGGTCTCtGGCAtaagtcg |
| | acaatcaacctctggattacaaaatttgtgaaagattgactggtattcttaacta |
| | tgttgctccttttacgctatgtggatacgctgctttaatgcctttgtatcatgct |
| | attgcttcccgtatggctttcattttctcctccttgtataaatcctggttgctgt |
| | ctctttatgaggagttgtggcccgttgtcaggcaacgtggcgtggtgtgcactgt |
| | gtttgctgacgcaacccccactggttggggcattgccaccacctgtcagctcctt |
| | tccgggactttcgctttccccctccctattgccacggcggaactcatcgccgcct |
| | gccttgcccgctgctggacaggggctcggctgttgggcactgacaattccgtggt |
| | gttgtcggggaaatcatcgtcctttccttggctgctcgcctgtgttgccacctgg |
| | attctgcgcgggacgtccttctgctacgtcccttcggccctcaatccagcggacc |
| | ttccttcccgcggcctgctgccggctctgcggcctcttccgcgtctAcgccttcg |
| | ccctcagacgagtcggatctccctttgggccgcctccccgcgtcgactttaagac |
| | caatgacttacaaggcagctgtagatcttagccactttttaaaagaaaagggggg |
| | actggaagggctaattcactcccaacgaagTcaagatctgctttttgcttgtact |
| | gggtctctctggttagaccagatctgagcctgggagctctctggctaactaggga |
| | acccactgcttaagcctcaataaagcttgccttgagtgcttcaagtagtgtgtgc |
| | ccgtctgttgtgtgactctggtaactagagatccctcagacccttttagtcagtg |
| | tggaaaatctctagcagggcccgtttaaacccgctgatcagcctcgactgtgcct |
| | tctagttgccagccatctgttgtttgcccctcccccgtgccttccttgaccctgg |
| | aaggtgccactcccactgtcctttcctaataaaatgaggaaattgcatcgcattg |
| | tctgagtaggtgtcattctattctggggggtggggggggcaggacagcaaggggg |
| | aggattgggaagagaa |
| | (SEQ ID NO: 35) |
| pAJW_R3 | tagcaggcatgctggggatgcggtgggctctatggcttctgaggcggaaagaacc |
| | agctggggctctagggggtatccccacgcgccctgtagcggcgcattaagcgcgg |
| | cgggtgtggtggttacgcgcagcgtgaccgctacacttgccagcgccctagcgcc |
| | cgctcctttcgctttcttcccttcctttctcgccacgttcgccggctttccccgt |
| | caagctctaaatcgggggctccctttagggttccgatttagtgctttacggcacc |
| | tcgaccccaaaaaacttgattagggtgatggttcacgtagtgggccatcgccctg |
| | atagacggtttttcgccctttgacgttggagtccacgttctttaatagtggactc |
| | ttgttccaaactggaacaacactcaaccctatctcggtctattcttttgatttat |
| | aagggattttgccgatttcggcctattggttaaaaaatgagctgatttaacaaaa |
| | atttaacgcgaattaattctgtggaatgtgtgtcagttagggtgtggaaagtccc |
| | caggctccccagcaggcagaagtatgcaaagcatgcatctcaattagtcagcaac |
| | caggtgtggaaagtccccaggctccccagcaggcagaagtatgcaaagcatgcat |
| | ctcaattagtcagcaaccatagtcccgcccctaactccgcccatcccgcccctaa |
| | ctccgcccagttccgcccattctccgccccatggctgactaattttttttattta |
| | tgcagaggccgaggccgcctctgcctctgagctattccagaagtagtgaggaggc |
| | ttttttggaggcctaggcttttgcaaaaagctcccgggagcttgtatatccattt |
| | tcggatctgatcagcacgtgttgacaattaatcatcggcatagtatatcggcata |
| | gtataatacgacaaggtgaggaactaaaccatggccaagttgaccagtgccgttc |
| | cggtgctcaccgcgcgcgacgtcgccggagcggtcgagttctggaccgaccggct |
| | cgggttctcccgggacttcgtggaggacgacttcgccggtgtggtccgggacgac |
| | gtgaccctgttcatcagcgcggtccaggaccaggtggtgccggacaacaccctgg |
| | cctgggtgtgggtgcgcggcctggacgagctgtacgccgagtggtcggaggtcgt |
| | gtccacgaacttccgggacgcctccgggccggccatgaccgagatcggcgagcag |
| | ccgtgggggcgggagttcgccctgcgcgacccggccggcaactgcgtgcacttcg |
| | tggccgaggagcaggactgacacgtgctacgagatttcgattccaccgccgcctt |
| | ctatgaaaggttgggcttcggaatcgttttccgggacgccggctggatgatcctc |
| | cagcgcggggatctcatgctggagttcttcgcccaccccaacttgtttattgcag |
| | cttataatggttacaaataaagcaatagcatcacaaatttcacaaataaagcatt |
| | tttttcactgcattctagttgtggtttgtccaaactcatcaatgtatcttatcat |
| | gtctgtataccgtcgacctctagctagagcttggcgtaatcatggtcatagctgt |
| | ttcctgtgtgaaattgttatccgctcacaattccacacaacatacgagccggaag |
| | cataaagtgtaaagcctggggtgcctaatgagtgagctaactcacattaattgcg |
| | ttgcgctcactgcccgctttccagtcgggaaacctgtcgtgccagctgcattaat |
| | gaatcggccaacgcgcggggagaggcggtttgcgtattgggcgctcttccgcttc |
| | ctcgctcactgactcgctgcgctcggtcgttcggctgcggcgagcggtatcagct |
| | cactcaaaggcggtaatacggttatccacagaatcaggggataacgcaggaaaga |
| | acatgtgagcaaaaggccagcaaaaggccaggaaccgtaaaaaggccgcgttgct |
| | ggcgtttttccataggctccgcccccctgacgagcatcacaaaaatcgacgctca |
| | agtcagaggtggcgaaacccgacaggactataaagataccaggcgtttccccctg |
| | gaagctccctcgtgcgctctcctgttccgaccctgccgcttaccggatacctgtc |
| | cgcctttctcccttcgggaagcgtggcgctttctcatagctcacgctgtaggtat |
| | ctcagttcggtgtaggtcgttcgctccaagctgggctgtgtgcacgaaccccccg |
| | ttcagcccgaccgctgcgccttatccggtaactatcgtcttgagtccaacccggt |
| | aagacacgacttatcgccactggcagcagccactggtaacaggattagcagagcg |
| | aggtatgtaggcggtgctacagagttcttgaagtggtggcctaactacggctaca |
| | ctagaagaacagtatttggtatctgcgctctgctgaagccagttaccttcggaaa |
| | aagagttggtagctcttgatccggcaaacaaaccaccgctggtagcggtggtttt |
| | tttgtttgcaagcagcagattacgcgcagaaaaaaaggatctcaagaagatcctt |
| | tgatcttttctacggggtctgacgctcagtggaacgaaaactcacgttaagggat |
| | tttggtcatgagattatcaaaaaggatcttcacctagatccttttaaattaaaaa |
| | tgaagttttaaatcaatctaaagtatatatgagtaaacttggtctgacagttacc |
| | aatgcttaatcagtgaggcacctatctcagcgatctgtctatttcgttcatccat |
| | agttgcctgactccccgtcgtgtagataactacgatacgggagggcttaccatct |
| | ggccccagtgctgcaatgataccgcgagacccacgctcaccggctccagatttat |
| | cagcaataaaccagccagccggaagggccgagcgcagaagtggtcctgcaacttt |
| | atccgcctccatccagtctattaattgttgccgggaagctagagtaagtagttcg |
| | ccagttaatagtttgcgcaacgttgttgccattgctacaggcatcgtggtgtcac |
| | gctcgtcgtttggtatggcttcattcagctccggttcccaacgatcaaggcgagt |
| | tacatgatcccccatgttgtgcaaaaaagcggttagctccttcggtcctccgatc |
| | gttgtcagaagtaagttggccgcagtgttatcactcatggttatggcagcactgc |
| | ataattctcttactgtcatgccatccgtaagatgcttttctgtgactggtgagta |
| | ctcaaccaagtcattctgagaatagtgtatgcggcgaccgagttgctcttgcccg |
| | gcgtcaatacgggataataccgcgccacatagcagaactttaaaagtgctcatca |
| | ttggaaaacgttcttcggggcgaaaactctcaaggatcttaccgctgttgagatc |
| | cagttcgatgtaacccactcgtgcacccaactgatcttcagcatcttttactttc |
| | accagcgtttctgggtgagcaaaaacaggaaggcaaaatgccgcaaaaaagggaa |
| | taagggcgacacggaaatgttgaatactcatactcttcctttttcaatattattg |
| | aagcatttatcagggttattgtctcatgagcggatacatatttgaatgtatttag |
| | aaaaataaacaaataggggttccgcgcacatttccccgaaaagtgccacctgacg |
| | tcgacggatcgggagatctcccgatcccctatggtgcactctcagtacaatctgc |
| | tctgatgccgcatagttaagccagtatctgctccctgcttgtgtgttggaggtcg |
| | ctgagtagtgcgcgagcaaaatttaagctacaacaaggcaaggcttgaccgacaa |
| | ttgcatgaagaatctgcttagggttaggcgttttgcgctgcttcgcgatgtacgg |
| | gccagatatacgcgttgacattgattattgactagttattaatagtaatcaatta |
| | cggggtcattagttcatagcccatatatggagttccgcgttacataacttacggt |
| | aaatggcccgcctggctgaccgcccaacgacccccgcccattgacgtcaataatg |
| | acgtatgttcccatagtaacgccaatagggactttccattgacgtcaatgggtgg |
| | agtatttacggtaaactgcccacttggcagtacatcaagtgtatcatatgccaag |
| | tacgccccctattgacgtcaatgacggtaaatggcccgcctggcattatgcccag |
| | tacatgaccttatgggactttcctacttggcagtacatctacgtattagtcatcg |
| | ctattaccatggtgatgcggttttggcagtacatcaatgggcgtggatagcggtt |
| | tgactcacggggatttccaagtctccaccccattgacgtcaatgggagtttgttt |
| | tggcaccaaaatcaacgggactttccaaaatgtcgtaacaactccgccccattga |
| | cgcaaatgggcggtaggcgtgtacggtgggaggtctatataagcagcgcgttttg |
| | cctgtactgggtctctctggttagaccagatctgagcctgggagctctctggcta |
| | actagggaacccactgcttaagcctcaataaagcttgccttgagtgcttcaagta |
| | gtgtgtgcccgtctgttgtgtgactctggtaactagagatccctcagaccctttt |
| | agtcagtgtggaaaatctctagcagtggcgcccgaacagggacttgaaagcgaaa |
| | gggaaaccagaggagctctctcgacgcaggactcggcttgctgaagcgcgcacgg |
| | caagaggcgaggggcggcgactggtgagtacgccaaaaattttgactagcggagg |
| | ctagaaggagagagatgggtgcgagagcgtcagtattaagcgggggagaattaga |
| | tcgcgatgggaaaaaattcggttaaggccagggggaaagaaaaaatataaattaa |
| | aacatatagtatgggcaagcagggagctagaacgattcgcagttaatcctggcct |
| | gttagaaacatcagaaggctgtagacaaatactgggacagctacaaccatccctt |
| | cagacaggatcagaagaacttagatcattatataatacagtagcaaccctctatt |
| | gtgtgcatcaaaggatagagataaaagacaccaaggaagctttagacaagataga |
| | ggaagagcaaaacaaaagtaagaccaccgcacagcaagcggccgctgatcttcag |
| | acctggaggaggagatatgagggacaattggagaagtgaattatataaatataaa |
| | gtagtaaaaattgaaccattaggagtagcacccaccaaggcaaagagaagagtgg |
| | tgcagagagaaaaaagagcagtgggaataggagctttgttccttgggttcttggg |
| | agcagcaggaagcactatgggcgcagcgtcaatgacgctgacggtacaggccaga |
| | caattattgtctggtatagtgcagcagcagaacaatttgctgagggctattgagg |
| | cgcaacagcatctgttgcaactcacagtctggggcatcaagcagctccaggcaag |
| | aatcctggctgtggaaagatacctaaaggatcaacagctcctggggatttggggt |
| | tgctctggaaaactcatttgcaccactgctgtgccttggaatgctagttggagta |
| | ataaatctctggaacagatttggaatcacacgacctggatggagtgggacagaga |
| | aattaacaattacacaagcttaatacactccttaattgaagaatcgcaaaaccag |
| | caagaaaagaatgaacaagaattattggaattagataaatgggcaagtttgtgga |
| | attggtttaacataacaaattggctgtggtatataaaattattcataatgatagt |
| | aggaggcttggtaggtttaagaatagtttttgctgtactttctatagtgaataga |
| | gttaggcagggatattcaccattatcgtttcagacccacctcccaaccccgaggg |
| | gacccgacaggcccgaaggaatagaagaagaaggtggagagagagacagagacag |
| | atccattcgattagtgaacggatcggcactgcgtgcgccaattctgcagacaaat |
| | ggcagtattcatccacaattttaaaagaaaaggggggattggggggtacagtgca |
| | ggggaaagaatagtagacataatagcaacagacatacaaactaaagaattacaaa |
| | aacaaattacaaaaattcaaaattttcgggtttattacagggacagcagagatcc |
| | agtttggttaattaaggtACCGGAGCGTTttacttgtacagctcgtccatgccga |
| | gagtgatcccggcggcggtcacgaactccagcaggaccatgtgatcgcgcttctc |
| | gttggggtctttgctcagggcggactgggtgctcaggtagtggttgtcgggcagc |
| | agcacggggccgtcgccgatgggggtgttctgctggtagtggtcggcgagctgca |
| | cgctgccgtcctcgatgttgtggcggatcttgaagttcaccttgatgccgttctt |
| | ctgcttgtcggccatgatatagacgttgtggctgttgtagttgtactccagcttg |
| | tgccccaggatgttgccgtcctccttgaagtcgatgcccttcagctcgatgcggt |
| | tcaccagggtgtcgccctcgaacttcacctcggcgcgggtcttgtagttgccgtc |
| | gtccttgaagaagatggtgcgctcctggacgtagccttcgggcatggcggacttg |
| | aagaagtcgtgctgcttcatgtggtcggggtagcggctgaagcactgcacgccgt |
| | aggtcagggtggtcacgagggtgggccagggcacgggcagcttgccggtggtgca |
| | gatgaacttcagggtcagcttgccgtaggtggcatcgccctcgccctcgccggac |
| | acgctgaacttgtggccgtttacgtcgccgtccagctcgaccaggatgggcacca |
| | ccccggtgaacagctcctcgcccttgctcaccatAGCCATGGTGGCGGCATTCgc |
| | ttggttctccctatagtgagtcgtattaatttcgataagccagtaagcagtgggt |
| | tctctagttagccagagagctctgcttatatagacctcccaccgtacacgcctac |
| | cgcccatttgcgtcaatggggcggagttgttacgacattttggaaagtcccgttg |
| | attttggtgccaaaacaaactcccattgacgtcaatggggggagacttggaaatc |
| | cccgtgagtcaaaccgctatccacgcccattgatgtactgccaaaaccgcatcac |
| | catggtaatagcgatgactaatacgtagatgtactgccaagtaggaaagtcccat |
| | aaggtcatgtactgggcataatgccaggcgggccatttaccgtcattgacgtcaa |
| | tagggggcgtacttggcatatgatacacttgatgtactgccaagtgggcagttta |
| | ccgtaaatactccacccattgacgtcaatggaaagtccctattggcgttactatg |
| | ggaacatacgtcattattgacgtcaatgggcgggggtcgttgggcggtcagccag |
| | gcgggccatttaccgtaagttatgtaacgcggaactccatatatgggctatgaac |
| | taatgaccccgtaattgattactattaataactagtcaataatcaatgtcaacga |
| | gtatatctggTTTGagtggttttggggtttttccataggatattggaactggtgg |
| | aattttgggagttaaccagcttaaccccagttcaaaggtatgaagtgacttcttc |
| | aaaacaaaccaaaatgaacacactttcccaccaaagaatattagtttagaattga |
| | acgtttgttcaccctgtgtaaaccaaTGCTGCCCCCTAGTGGCGAaggtggcaag |
| | ttcaagcaagtcaccttgagggccaggaaattagtaattaggctaatataatttc |
| | ccaaagagcattgcaaaagaGCGAGGCATCGCCAAAggatctgcgatcgctccgg |
| | tgcccgtcagtgggcagagcgcacatcgcccacagtccccgagaagttgggggga |
| | ggggtcggcaattgaacgggtgcctagagaaggtggcgcggggtaaactgggaaa |
| | gtgatgtcgtgtactggctccgcctttttcccgagggtgggggagaaccgtatat |
| | aagtgcagtagtcgccgtgaacgttctttttcgcaacgggtttgccgccagaaca |
| | cagctgaagcttcgaggggctcgcatctctccttcacgcgcccgccgccctacct |
| | gaggccgccatccacgccggttgagtcgcgttctgccgcctcccgcctgtggtgc |
| | ctcctgaactgcgtccgccgtctaggtaagtttaaagctcaggtcgagTccgggc |
| | ctttgtccggcgctcccttggagcctacctagactcagccggctctccacgcttt |
| | gcctgaccctgcttgctcaactctacgtctttgtttcgttttctgttctgcgccg |
| | ttacagatccaagctgtgaccggcgcctacGAATGCCGCCACCATGGCTaccgag |
| | tataaaccgacggtccgacttgcgactcgggatgacgttccacgcgccgtgagga |
| | ctctggccgccgccttcgctgactacccggccacacggcacacagttgaccccga |
| | cagacacatagagcgggtcacagagttgcaagaacttttcctgacgcgggttggA |
| | ctcgatattggaaaagtttgggttgccgacgatggtgccgcagttgcggtgtgga |
| | ctactccagaaagtgtagaagctggagccgtattcgctgaaatagggccacggat |
| | ggcagagctgtctggtagcaggctcgcagcgcagcagcagatggaaggCcttctc |
| | gctccgcatcggcctaaggaaccggcgtggttcctggctacggtgggagtctcac |
| | ctgaccatcaaggaaagggattgggaagtgctgtcgttcttccaggcgtggaagc |
| | ggccgaacgagcaggcgtcccagccttcttggaAacgagcgcacccaggaatctt |
| | ccattctacgagcgactgggtttcactgtgacagcagacgtagaagtccctgaag |
| | ggcctcggacttggtgcatgaccagaaagcctggggcttgaAACGTCGCaatcaa |
| | cctctggattacaaaatttgtgaaagattgactggtattcttaactatgttgctc |
| | cttttacgctatgtggatacgctgctttaatgcctttgtatcatgctattgcttc |
| | ccgtatggctttcattttctcctccttgtataaatcctggttgctgtctctttat |
| | gaggagttgtggcccgttgtcaggcaacgtggcgtggtgtgcactgtgtttgctg |
| | acgcaacccccactggttggggcattgccaccacctgtcagctcctttccgggac |
| | tttcgctttccccctccctattgccacggcggaactcatcgccgcctgccttgcc |
| | cgctgctggacaggggctcggctgttgggcactgacaattccgtggtgttgtcgg |
| | ggaaatcatcgtcctttccttggctgctcgcctgtgttgccacctggattctgcg |
| | cgggacgtccttctgctacgtcccttcggccctcaatccagcggaccttccttcc |
| | cgcggcctgctgccggctctgcggcctcttccgcgtctAcgccttcgccctcaga |
| | cgagtcggatctccctttgggccgcctccccgcgtcgactttaagaccaatgact |
| | tacaaggcagctgtagatcttagccactttttaaaagaaaaggggggactggaag |
| | ggctaattcactcccaacgaagTcaagatctgctttttgcttgtactgggtctct |
| | ctggttagaccagatctgagcctgggagctctctggctaactagggaacccactg |
| | cttaagcctcaataaagcttgccttgagtgcttcaagtagtgtgtgcccgtctgt |
| | tgtgtgactctggtaactagagatccctcagacccttttagtcagtgtggaaaat |
| | ctctagcagggcccgtttaaacccgctgatcagcctcgactgtgccttctagttg |
| | ccagccatctgttgtttgcccctcccccgtgccttccttgaccctggaaggtgcc |
| | actcccactgtcctttcctaataaaatgaggaaattgcatcgcattgtctgagta |
| | ggtgtcattctattctggggggtggggggggcaggacagcaagggggaggattgg |
| | gaagagaa |
| | (SEQ ID NO: 36) |
| psPAX2 | See addgene plasmid #12260 |
| pMD2.G | See addgene plasmid #12259 |
| pAJW_P99 | TTTTATCTCTCATTCGAAAATTCCCAACATCGGGATTATATAACTGAAAAGTTTT |
| (FUT7 + PSGL-1) | GGCGTAATGCGTTGGTCGCAGGAACCGTACCCGTAGTCCTCGGACCACCCCGCGC |
| | AACTTACGAGGCTTTTGTTCCCGCAGACGCATTCGTACACGTGGACGATTTCGGT |
| | TCCGCTCGGGAACTCGCTGCATTTCTGACAGGTATGAACGAATCTAGGTATCAGC |
| | GGTTCTTCGCGTGGCGGGATCGGCTTCGGGTCAGGTTGTTTACGGATTGGAGAGA |
| | GAGATTTTGCGCAATATGCGATAGGTATCCTCATCTGCCTAGATCCCAGGTGTAC |
| | GAAGATTTGGAAGGCTGGTTCCAAGCATAGAACGCCATgggtggcatccctgtga |
| | cccctccccagtgcctctcctggccctggaagttgccactccagtgcccaccagc |
| | cttgtcctaataaaattaagttgcatcattttgtctgactaggtgtccttctata |
| | atattatggggtggaggggggtggtatggagcaaggggcaagttgggaagagaac |
| | ctgtagggcctgcggggtctattgggaaccaagctggagtgcagtggcacaatct |
| | tggctcactgcaatctccgcctcctgggttcaagcgattctcctgcctcagcctc |
| | ccgagttgttgggattccaggcatgcatgaccaggctcagctaatttttgttttt |
| | ttggtagagTcggggtttcaccatattggccaggctggtgtccaactcctaatct |
| | caggtgatctacccaccttggcctcccaaattgctgggattacaggcgtgaacca |
| | ctgctcccttccctgtccttCAGTGGCATCGCtcttttgcaatgctctttgggaa |
| | attatattagcctaattactaatttcctggccctcaaggtgacttgcttgaactt |
| | gccacctTCGCCACTAGGGGGCAGCAttggtttacacagggtgaacaaacgttca |
| | attctaaactaatattctttggtgggaaagtgtgttcattttggtttgttttgaa |
| | gaagtcacttcatacctttgaactggggttaagctggttaactcccaaaattcca |
| | ccagttccaatatcctatggaaaaaccccaaaaccactCAAAggatctgcgatcg |
| | ctccggtgcccgtcagtgggcagagcgcacatcgcccacagtccccgagaagttg |
| | gggggaggggtcggcaattgaacgggtgcctagagaaggtggcgcggggtaaact |
| | gggaaagtgatgtcgtgtactggctccgcctttttcccgagggtgggggagaacc |
| | gtatataagtgcagtagtcgccgtgaacgttctttttcgcaacgggtttgccgcc |
| | agaacacagctgaagcttcgaggggctcgcatctctccttcacgcgcccgccgcc |
| | ctacctgaggccgccatccacgccggttgagtcgcgttctgccgcctcccgcctg |
| | tggtgcctcctgaactgcgtccgccgtctaggtaagtttaaagctcaggtcgagT |
| | ccgggcctttgtccggcgctcccttggagcctacctagactcagccggctctcca |
| | cgctttgcctgaccctgcttgctcaactctacgtctttgtttcgttttctgttct |
| | gcgccgttacagatccaagctgtgaccggcgcctacGAATGCCGCCACCATGCCC |
| | CTTCAGCTGTTGTTGCTTTTGATACTGCTGGGACCTGGAAACTCACTTCAGTIGT |
| | GGGACACATGGGCCGATGAGGCGGAGAAGGCCCTGGGTCCCCTTCTCGCAAGAGA |
| | CAGACGGCAAGCGACTGAGTATGAATACCTCGATTATGATTTCCTCCCCGAGACt |
| | GAGCCTCCTGAGATGTTGCGCAACAGTACAGACACAACGCCATTGACTGGCCCCG |
| | GAACTCCAGAGAGCACCACGGTTGAGCCTGCTGCCCGGAGAAGTACTGGATTGGA |
| | TGCGGGCGGCGCTGTGACTGAATTGACCACTGAGCTGGCCAATATGGGGAATCTG |
| | TCCACAGACAGCGCGGCCATGGAGATACAAACGACACAGCCTGCGGCCACCGAGG |
| | CCCAGACTACACAGCCTGTACCGACAGAGGCCCAAACTACACCATTGGCAGCTAC |
| | GGAAGCTCAaACaACTCGAttgACcGCtACCGAgGCACAGACTACCCCTCTCGCT |
| | GCGACtGAAGCcCAaACGACaCCACCTGCaGCaACaGAAGCACAAACTACACAGC |
| | CCACAGGGTTGGAGGCGCAAACAACTGCGCCTGCTGCCATGGAAGCTCAAACAAC |
| | GGCACCGGCCGCAATGGAGGCaCAGACTACtCCtCCtGCaGCTATGGAGGCcCAA |
| | ACGACaCAGACGACTGCcATGGAGGCACAGACAACTGCACCAGAGGCCACGGAGG |
| | CACAAACAACACAACCAACTGCAACAGAGGCGCAGACGACGCCCCTCGCAGCAAT |
| | GGAAGCTCTTTCCACGGAACCCAGCGCAACCGAAGCACTCTCTATGGAGCCTACA |
| | ACCAAACGGGGCCTTTTTATTCCATTCTCTGTGTCATCCGTGACTCATAAGGGGA |
| | TACCTATGGCTGCGTCTAATCTCTCAGTCAATTATCCAGTTGGTGCGCCAGATCA |
| | CATTTCCGTGAAACAGTGTCTCCTTGCCATATTGATACTTGCCTTGGTCGCGACT |
| | ATTTTTTTCGTGTGTACCGTAGTGCTCGCGGTTCGCCTCTCTCGGAAGGGTCACA |
| | TGTACCCTGTTAGGAATTATTCTCCCACTGAAATGGTGTGCATCAGCAGTCTGCT |
| | GCCTGATGGTGGCGAGGGACCATCCGCTACAGCCAACGGAGGACTCTCTAAGGCT |
| | AAGTCTCCTGGACTGACCCCTGAGCCCCGCGAGGATAGGGAAGGtGACGATCTCA |
| | CCCTCCACTCCTTTCTGCCAtagAACGCCATgaattcactcctcaggtgcaggct |
| | gcctatcagaaggtggtggctggtgtggccaatgccctggctcacaaataccact |
| | gagatctttttccctctgccaaaaattatggggacatcatgaagccccttgagca |
| | tctgacttctggctaataaaggaaatttattttcattgcaatagtgtgttggaat |
| | tttttgtgtctctcactcggaaggacatatgggagggcaaatcatttaaaacatc |
| | agaatgagtatttggtttagagtttggcaacatatgcccatatgctggctgccat |
| | gaacaaaggttggctataaagaggtcatcagtatatgaaacagccccctgctgtc |
| | cattccttattccatagaaaagccttgacttgaggttagattttttttatatttt |
| | gttttgtgttatttttttctttaacatccctaaaattttccttacatgttttact |
| | agccagatttttcctcctctcctgactactcccagtcatagctgtccctcttctc |
| | ttatggagatCAGTTCGCTCGCtcttttgcaatgctctttgggaaattatattag |
| | cctaattactaatttcctggccctcaaggtgacttgcttgaacttgccacctTCG |
| | CCACTAGGGGGCAGCAttggtttacacagggtgaacaaacgttcaattctaaact |
| | aatattctttggtgggaaagtgtgttcattttggtttgttttgaagaagtcactt |
| | catacctttgaactggggttaagctggttaactcccaaaattccaccagttccaa |
| | tatcctatggaaaaaccccaaaaccactCAAAggatctgcCatcgctccggtgcc |
| | cgtcagtgggcagagcgcacatcgcccacagtccccgagaagttggggggagggg |
| | tcggcaattgaacgggtgcctagagaaggtggcgcggggtaaactgggaaagtga |
| | tgtcgtgtactggctccgcctttttcccgaggggggggagaaccgtatataagtg |
| | cagtagtcgccgtgaacgttctttttcgcaacgggtttgccgccagaacacagct |
| | gaagcttcgaggggctcgcatctctccttcacgcgcccgccgccctacctgaggc |
| | cgccatccacgccggttgagtcgcgttctgccgcctcccgcctgtggtgcctcct |
| | gaactgcgtccgccgtctaggtaagtttaaagctcaggtcgagTccgggcctttg |
| | tccggcgctcccttggagcctacctagactcagccggctctccacgctttgcctg |
| | accctgcttgctcaactctacgtctttgtttcgttttctgttctgcgccgttaca |
| | gatccaagctgtgaccggcgcctacGAATGCCGCCACCATGGCTgctgaaggTtc |
| | cgtcgccaggcagcctgacctcttgacctgcgacgatgagccgatccatatcccc |
| | ggtgccatccaaccgcatggactgctgctcgccctcgccgccgacatgacgatcg |
| | ttgccggcagcgacaaccttcccgaactcaccggactggcgatcggcgccctgat |
| | cggccgctctgcggccgatgtGttcgactcggaAacgcacaaccgtctgacgatc |
| | gccttggccgagcccggggcggccgtcggagcaccgatcactgtcggcttcacga |
| | tgcgaaaggacgcaggcttcatcggctcctggcatcgccatgatcagctcatctt |
| | cctGgagctGgagcctccccagcgggacgtcgccgagccgcaggcgttcttccgc |
| | cgcaccaacagcgccatccgccgcctgcaggccgccgaaaccttggaaagcgcct |
| | gcgccgccgcggcgcaagaggtgcggaagattaccggcttcgatcgggtgatgat |
| | ctatcgcttcgcctccgacttcagcggcgaagtgatcgcagaggatcggtgcgcc |
| | gaggtcgagtcaaaactaggcctgcactatcctgcctcaaccgtgccggcgcagg |
| | cccgtcggctctataccatcaacccggtacggatcattcccgatatcaattatcg |
| | gccggtgccggtcaccccagacctcaatccggtcaccgggcggccgattgatctt |
| | agcttcgccatcctgcgcagcgtGtcgcccgtccatctggaGttcatgcgcaaca |
| | taggcatgcacggcacgatgtcgatctcgattttgcgcggcgagcgactgtgggg |
| | attgatcgtttgccatcaccgaacgccgtactacgtcgatctcgatggccgccaa |
| | gcctgcgagctagtcgcccaggttctggcctggcagatcggcgtgatggaagagT |
| | AAAACGBBABBABBABBABBABBACCATctgtgccttctagttgccagccatctg |
| | ttgtttgcccctcccccgtgccttccttgaccctggaaggtgccactcccactgt |
| | cctttcctaataaaatgaggaaattgcatcgcattgtctgagtaggtgtcattct |
| | attctggggggtggggtggggcaggacagcaagggggaggattgggaaggcaata |
| | gcaggcatgctggggatgcggtgggctctatggCAGTCAGTGCGGCCGCGCCTGG |
| | GCTTACttaattaacgagagcataatattgatatgtgccaaagttgtttctgact |
| | gactaataagtataatttgtttctattatgtataggttaagctaattacttattt |
| | tataatacaacatgactgtttttaaagtacaaaataagtttatttttgtaaaaga |
| | gagaatgtttaaaagttttgttactttatagaagaaattttgagtttttgttttt |
| | ttttaataaataaataaacataaataaattgtttgttgaatttattattagtatg |
| | taagtgtaaatataataaaacttaatatctattcaaattaataaataaacctcga |
| | tatacagaccgataaaacacatgcgtcaattttacgcatgattatctttaacgta |
| | cgtcacaatatgattatctttctagggttaaataatagtttctaatttttttatt |
| | attcagcctgctgtcgtgaataccgagctccaattcgccctatagtgagtcgtat |
| | tacaattcactggccgtcgttttacaacgtcgtgactgggaaaaccctggcgtta |
| | cccaacttaatcgccttgcagcacatccccctttcgccagctggcgtaatagcga |
| | agaggcccgcaccgatcgcccttcccaacagttgcgcagcctgaatggcgaatgg |
| | gacgcgccctgtagcggcgcattaagcgcggcgggtgtggtggttacgcgcagcg |
| | tgaccgctacacttgccagcgccctagcgcccgctcctttcgctttcttcccttc |
| | ctttctcgccacgttcgccggctttccccgtcaagctctaaatcgggggctccct |
| | ttagggttccgatttagtgctttacggcacctcgaccccaaaaaacttgattagg |
| | gtgatggttcacgtagtgggccatcgccctgatagacggtttttcgccctttgac |
| | gttggagtccacgttctttaatagtggactcttgttccaaactggaacaacactc |
| | aaccctatctcggtctattcttttgatttataagggattttgccgatttcggcct |
| | attggttaaaaaatgagctgatttaacaaaaatttaacgcgaattttaacaaaat |
| | attaacgcttacaatttaggtggcacttttcggggaaatgtgcgcggaaccccta |
| | tttgtttatttttctaaatacattcaaatatgtatccgctcatgagacaataacc |
| | ctgataaatgcttcaataatattgaaaaaggaagagtatgagtattcaacatttc |
| | cgtgtcgcccttattcccttttttgcggcattttgccttcctgtttttgctcacc |
| | cagaaacgctggtgaaagtaaaagatgctgaagatcagttgggtgcacgagtggg |
| | ttacatcgaactggatctcaacagcggtaagatccttgagagttttcgccccgaa |
| | gaacgttttccaatgatgagcacttttaaagttctgctatgtggcgcggtattat |
| | cccgtattgacgccgggcaagagcaactcggtcgccgcatacactattctcagaa |
| | tgacttggttgagtactcaccagtcacagaaaagcatcttacggatggcatgaca |
| | gtaagagaattatgcagtgctgccataaccatgagtgataacactgcggccaact |
| | tacttctgacaacgatcggaggaccgaaggagctaaccgcttttttgcacaacat |
| | gggggatcatgtaactcgccttgatcgttgggaaccggagctgaatgaagccata |
| | ccaaacgacgagcgtgacaccacgatgcctgtagcaatggcaacaacgttgcgca |
| | aactattaactggcgaactacttactctagcttcccggcaacaattaatagactg |
| | gatggaggcggataaagttgcaggaccacttctgcgctcggcccttccggctggc |
| | tggtttattgctgataaatctggagccggtgagcgtgggtctcgcggtatcattg |
| | cagcactggggccagatggtaagccctcccgtatcgtagttatctacacgacggg |
| | gagtcaggcaactatggatgaacgaaatagacagatcgctgagataggtgcctca |
| | ctgattaagcattggtaactgtcagaccaagtttactcatatatactttagattg |
| | atttaaaacttcatttttaatttaaaaggatctaggtgaagatcctttttgataa |
| | tctcatgaccaaaatcccttaacgtgagttttcgttccactgagcgtcagacccc |
| | gtagaaaagatcaaaggatcttcttgagatcctttttttctgcgcgtaatctgct |
| | gcttgcaaacaaaaaaaccaccgctaccagcggtggtttgtttgccggatcaaga |
| | gctaccaactctttttccgaaggtaactggcttcagcagagcgcagataccaaat |
| | actgttcttctagtgtagccgtagttaggccaccacttcaagaactctgtagcac |
| | cgcctacatacctcgctctgctaatcctgttaccagtggctgctgccagtggcga |
| | taagtcgtgtcttaccgggttggactcaagacgatagttaccggataaggcgcag |
| | cggtcgggctgaacggggggttcgtgcacacagcccagcttggagcgaacgacct |
| | acaccgaactgagatacctacagcgtgagctatgagaaagcgccacgcttcccga |
| | agggagaaaggcggacaggtatccggtaagcggcagggtcggaacaggagagcgc |
| | acgagggagcttccagggggaaacgcctggtatctttatagtcctgtcgggtttc |
| | gccacctctgacttgagcgtcgatttttgtgatgctcgtcaggggggcggagcct |
| | atggaaaaacgccagcaacgcggcctttttacggttcctggccttttgctggcct |
| | tttgctcacatgttctttcctgcgttatcccctgattctgtggataaccgtatta |
| | ccgcctttgagtgagctgataccgctcgccgcagccgaacgaccgagcgcagcga |
| | gtcagtgagcgaggaagcggaagagcgcccaatacgcaaaccgcctctccccgcg |
| | cgttggccgattcattaatgcagctggcacgacaggtttcccgactggaaagcgg |
| | gcagtgagcgcaacgcaattaatgtgagttagctcactcattaggcaccccaggc |
| | tttacactttatgcttccggctcgtatgttgtgtggaattgtgagcggataacaa |
| | tttcacacaggaaacagctatgaccatgattacgccaagctcgaaattaaccctc |
| | actaaagggaacaaaagctggtacctcgcgcgacttggtttgccattctttagcg |
| | cgcgtcgcgtcacacagcttggccacaatgtggtttttgtcaaacgaagattcta |
| | tgacgtgtttaaagtttaggtcgagtaaagcgcaaatcttttttaaccctagaaa |
| | gatagtctgcgtaaaattgacgcatgcattcttgaaatattgctctctctttcta |
| | aatagcgcgaatccgtcgctgtgcatttaggacatctcagtcgccgcttggagct |
| | cccgtgaggcgtgcttgtcaatgcggtaagtgtcactgattttgaactataacga |
| | ccgcgtgagtcaaaatgacgcatgattatcttttacgtgacttttaagatttaac |
| | tcatacgataattatattgttatttcatgttctacttacgtgataacttattata |
| | tatatattttcttgttatagatatcatcaactttgtatagaaaagttgctcgaca |
| | ttgattattgactagttattaatagtaatcaattacggggtcattagttcatagc |
| | ccatatatggagttccgcgttacataacttacggtaaatggcccgcctggctgac |
| | cgcccaacgacccccgcccattgacgtcaataatgacgtatgttcccatagtaac |
| | gccaatagggactttccattgacgtcaatgggtggagtatttacggtaaactgcc |
| | cacttggcagtacatcaagtgtatcatatgccaagtacgccccctattgacgtca |
| | atgacggtaaatggcccgcctggcattatgcccagtacatgaccttatgggactt |
| | tcctacttggcagtacatctacgtattagtcatcgctattaccatggtcgaggtg |
| | agccccacgttctgcttcactctccccatctcccccccctccccacccccaattt |
| | tgtatttatttattttttaattattttgtgcagcgatgggggcgggggggggggg |
| | ggggcgcgcgccaggcggggggggcggggcgaggggggggggggcgaggcggaga |
| | ggtgcggcggcagccaatcagagcggcgcgctccgaaagtttccttttatggcga |
| | ggcggcggcggcggcggccctataaaaagcgaagcgcgcggcgggcgggagtcgc |
| | tgcgcgctgccttcgccccgtgccccgctccgccgccgcctcgcgccgcccgccc |
| | cggctctgactgaccgcgttactcccacaggtgagcggggggacggcccttctcc |
| | tccgggctgtaattagcgcttggtttaatgacggcttgtttcttttctgtggctg |
| | cgtgaaagccttgaggggctccgggagggccctttgtgcggggggagcggctcgg |
| | ggggtgcgtgcgtgtgtgtgtgcgtggggagcgccgcgtgcggctccgcgctgcc |
| | cggcggctgtgagcgctgcgggcgcggcgcggggctttgtgcgctccgcagtgtg |
| | cgcgaggggagcgcggccgggggcggtgccccgcggtgcggggggggctgcgagg |
| | ggaacaaaggctgcgtgcggggtgtgtgcgtgggggggtgagcagggggtgtggg |
| | cgcgtcggtcgggctgcaaccccccctgcacccccctccccgagttgctgagcac |
| | ggcccggcttcgggtgcggggctccgtacggggcgtggcgcggggctcgccgtgc |
| | cgggcggggggggcggcaggtgggggtgccgggcggggcggggccgcctcgggcc |
| | ggggagggctcgggggaggggcgcggcggcccccggagcgccggcggctgtcgag |
| | gcgcggcgagccgcagccattgccttttatggtaatcgtgcgagagggcgcaggg |
| | acttcctttgtcccaaatctgtgcggagccgaaatctgggaggcgccgccgcacc |
| | ccctctagcgggcgcggggcgaagcggtgcggcgccggcaggaaggaaatgggcg |
| | gggagggccttcgtgcgtcgccgcgccgccgtccccttctccctctccagcctcg |
| | gggctgtccgcggggggacggctgccttcgggggggacggggcagggcggggttc |
| | ggcttctggcgtgtgaccggcggctctagagcctctgctaaccatgttcatgcct |
| | tcttctttttcctacagctcctgggcaacgtgctggttattgtgctgtctcatca |
| | ttttggcaaagaattgcaagtttgtacaaaaaagcaggctGAATGCCGCCACCAT |
| | GGCTaccgagtataaaccgacggtccgacttgcgactcgggatgacgttccacgc |
| | gccgtgaggactctggccgccgccttcgctgactacccggccacacggcacacag |
| | ttgaccccgacagacacatagagcgggtcacagagttgcaagaacttttcctgac |
| | gcgggttggActcgatattggaaaagtttgggttgccgacgatggtgccgcagtt |
| | gcggtgtggactactccagaaagtgtagaagctggagccgtattcgctgaaatag |
| | ggccacggatggcagagctgtctggtagcaggctcgcagcgcagcagcagatgga |
| | aggCcttctcgctccgcatcggcctaaggaaccggcgtggttcctggctacggtg |
| | ggagtctcacctgaccatcaaggaaagggattgggaagtgctgtcgttcttccag |
| | gcgtggaagcggccgaacgagcaggcgtcccagccttcttggaAacgagcgcacc |
| | caggaatcttccattctacgagcgactgggtttcactgtgacagcagacgtagaa |
| | gtccctgaagggcctcggacttggtgcatgaccagaaagcctggggcttgaAACG |
| | CCATgatccagacatgataagatacattgatgagtttggacaaaccacaactaga |
| | atgcagtgaGGCGCGCCTGGATACTTCTGCCTGCAGGGGAGTCGCtcttttgcaa |
| | tgctctttgggaaattatattagcctaattactaatttcctggccctcaaggtga |
| | cttgcttgaacttgccacctTCGCCACTAGGGGGCAGCAttggtttacacagggt |
| | gaacaaacgttcaattctaaactaatattctttggtgggaaagtgtgttcatttt |
| | ggtttgttttgaagaagtcacttcatacctttgaactggggttaagctggttaac |
| | tcccaaaattccaccagttccaatatcctatggaaaaaccccaaaaccactCAAA |
| | ggggttggggttgcgccttttccaaggcagccctgggtttgcgcagggacgcggc |
| | tgctctgggcgtggttccgggaaacgcagcggcgccgaccctgggTctGgcacat |
| | tcttcacgtccgttcgcagcgtcacccggatcttcgccgctacccttgtgggccc |
| | cccggcgacgcttcctgctccgcccctaagtcgggaaggttccttgcggttcgcg |
| | gcgtgccggacgtgacaaacggaagccgcacgTctGactagtaccctcgcagacg |
| | gacagcgccagggagcaatggcagcgcgccgaccgcgatgggctgtggccaatag |
| | cggctgctcagcAgggcgcgccgagagcagcggccgggaaggggcggtgcgggag |
| | gcggggtgtggggcggtagtgtgggccctgttcctgcccgcgcggtgttccgcat |
| | tctgcaagcctccggagcgcacgtcggcagtcggctccctcgttgaccgaatcac |
| | cgacctctctccccagGAATGCCGCCACCATGAATAACGCCGGCCACGGACCAAC |
| | TAGACGCTTGAGGGGACTCGGAGTTTTGGCTGGAGTCGCACTCCTGGCCGCTCTG |
| | TGGCTGCTTTGGCTCCTTGGAAGCGCGCCAAGGGGCACACCTGCACCACAACCTA |
| | CCATAACAATTCTGGTGTGGCATTGGCCATTTACCGATCAACCACCCGAATTGCC |
| | GTCAGATACATGTACCAGGTATGGGATTGCACGGTGTCATCTCAGCGCGAATCGC |
| | TCATTGTTGGCTTCAGCAGATGCGGTAGTGTTTCATCATAGAGAACTTCAAACTC |
| | GCAGGAGCCACCTTCCTTTGGCACAAAGGCCGCGGGGTCAACCGTGGGTTTGGGC |
| | ATCAATGGAAAGTCCCTCACATACTCATGGGCTGTCACATCTGCGGGGAATCTTT |
| | AATTGGGTACTTTCCTATCGACGGGATAGCGATATTTTCGTCCCATACGGACGAT |
| | TGGAACCTCATTGGGGTCCTTCTCCCCCTCTCCCTGCAAAATCCAGAGTTGCAGC |
| | TTGGGTCGTTTCCAATTTTCAAGAACGCCAACTTAGGGCTCGCCTCTATAGACAA |
| | CTCGCTCCCCACCTTCGTGTTGACGTTTTCGGTCGAGCTAACGGGCGCCCGTTGT |
| | GTGCGTCATGTCTCGTACCTACGGTTGCTCAATATCGG |
| | (SEQ ID NO: 37) |
| pAJW_P107 | tgcacgagtgggttacatcgaactggatctcaacagcggtaagatccttgagagt |
| (FUT7+PSGL- | tttcgccccgaagaacgttttccaatgatgagcacttttaaagttctgctatgtg |
| 1+ST3GAL4) | gcgcggtattatcccgtattgacgccgggcaagagcaactcggtcgccgcataca |
| | ctattctcagaatgacttggttgagtactcaccagtcacagaaaagcatcttacg |
| | gatggcatgacagtaagagaattatgcagtgctgccataaccatgagtgataaca |
| | ctgcggccaacttacttctgacaacgatcggaggaccgaaggagctaaccgcttt |
| | tttgcacaacatgggggatcatgtaactcgccttgatcgttgggaaccggagctg |
| | aatgaagccataccaaacgacgagcgtgacaccacgatgcctgtagcaatggcaa |
| | caacgttgcgcaaactattaactggcgaactacttactctagcttcccggcaaca |
| | attaatagactggatggaggcggataaagttgcaggaccacttctgcgctcggcc |
| | cttccggctggctggtttattgctgataaatctggagccggtgagcgtgggtctc |
| | gcggtatcattgcagcactggggccagatggtaagccctcccgtatcgtagttat |
| | ctacacgacggggagtcaggcaactatggatgaacgaaatagacagatcgctgag |
| | ataggtgcctcactgattaagcattggtaactgtcagaccaagtttactcatata |
| | tactttagattgatttaaaacttcatttttaatttaaaaggatctaggtgaagat |
| | cctttttgataatctcatgaccaaaatcccttaacgtgagttttcgttccactga |
| | gcgtcagaccccgtagaaaagatcaaaggatcttcttgagatcctttttttctgc |
| | gcgtaatctgctgcttgcaaacaaaaaaaccaccgctaccagcggtggtttgttt |
| | gccggatcaagagctaccaactctttttccgaaggtaactggcttcagcagagcg |
| | cagataccaaatactgttcttctagtgtagccgtagttaggccaccacttcaaga |
| | actctgtagcaccgcctacatacctcgctctgctaatcctgttaccagtggctgc |
| | tgccagtggcgataagtcgtgtcttaccgggttggactcaagacgatagttaccg |
| | gataaggcgcagcggtcgggctgaacggggggttcgtgcacacagcccagcttgg |
| | agcgaacgacctacaccgaactgagatacctacagcgtgagctatgagaaagcgc |
| | cacgcttcccgaagggagaaaggcggacaggtatccggtaagcggcagggtcgga |
| | acaggagagcgcacgagggagcttccagggggaaacgcctggtatctttatagtc |
| | ctgtcgggtttcgccacctctgacttgagcgtcgatttttgtgatgctcgtcagg |
| | ggggcggagcctatggaaaaacgccagcaacgcggcctttttacggttcctggcc |
| | ttttgctggccttttgctcacatgttctttcctgcgttatcccctgattctgtgg |
| | ataaccgtattaccgcctttgagtgagctgataccgctcgccgcagccgaacgac |
| | cgagcgcagcgagtcagtgagcgaggaagcggaagagcgcccaatacgcaaaccg |
| | cctctccccgcgcgttggccgattcattaatgcagctggcacgacaggtttcccg |
| | actggaaagcgggcagtgagcgcaacgcaattaatgtgagttagctcactcatta |
| | ggcaccccaggctttacactttatgcttccggctcgtatgttgtgtggaattgtg |
| | agcggataacaatttcacacaggaaacagctatgaccatgattacgccaagctcg |
| | aaattaaccctcactaaagggaacaaaagctggtacctcgcgcgacttggtttgc |
| | cattctttagcgcgcgtcgcgtcacacagcttggccacaatgtggtttttgtcaa |
| | acgaagattctatgacgtgtttaaagtttaggtcgagtaaagcgcaaatcttttt |
| | taaccctagaaagatagtctgcgtaaaattgacgcatgcattcttgaaatattgc |
| | tctctctttctaaatagcgcgaatccgtcgctgtgcatttaggacatctcagtcg |
| | ccgcttggagctcccgtgaggcgtgcttgtcaatgcggtaagtgtcactgatttt |
| | gaactataacgaccgcgtgagtcaaaatgacgcatgattatcttttacgtgactt |
| | ttaagatttaactcatacgataattatattgttatttcatgttctacttacgtga |
| | taacttattatatatatattttcttgttatagatatcatcaactttgtatagaaa |
| | agttgctcgacattgattattgactagttattaatagtaatcaattacggggtca |
| | ttagttcatagcccatatatggagttccgcgttacataacttacggtaaatggcc |
| | cgcctggctgaccgcccaacgacccccgcccattgacgtcaataatgacgtatgt |
| | tcccatagtaacgccaatagggactttccattgacgtcaatgggtggagtattta |
| | cggtaaactgcccacttggcagtacatcaagtgtatcatatgccaagtacgcccc |
| | ctattgacgtcaatgacggtaaatggcccgcctggcattatgcccagtacatgac |
| | cttatgggactttcctacttggcagtacatctacgtattagtcatcgctattacc |
| | atggtcgaggtgagccccacgttctgcttcactctccccatctcccccccctccc |
| | cacccccaattttgtatttatttattttttaattattttgtgcagcgatgggggc |
| | ggggggggggggggggcgcgcgccaggcggggcggggggggcgaggggggggcgg |
| | ggcgaggcggagaggtgcggggcagccaatcagagcggcgcgctccgaaagtttc |
| | cttttatggcgaggcggcggcggcggcggccctataaaaagcgaagcgcgcgggg |
| | ggggagtcgctgcgcgctgccttcgccccgtgccccgctccgccgccgcctcgcg |
| | ccgcccgccccggctctgactgaccgcgttactcccacaggtgagcggggggacg |
| | gcccttctcctccgggctgtaattagcgcttggtttaatgacggcttgtttcttt |
| | tctgtggctgcgtgaaagccttgaggggctccgggagggccctttgtgcgggggg |
| | agcggctcggggggtgcgtgcgtgtgtgtgtgcgtggggagcgccgcgtgcggct |
| | ccgcgctgcccggcggctgtgagcgctgcgggcgcggcgcggggctttgtgcgct |
| | ccgcagtgtgcgcgaggggagcgcggccgggggcggtgccccgcggtgcgggggg |
| | ggctgcgaggggaacaaaggctgcgtgcggggtgtgtgcgtgggggggtgagcag |
| | ggggtgtgggcgcgtcggtcgggctgcaaccccccctgcacccccctccccgagt |
| | tgctgagcacggcccggcttcgggtgcggggctccgtacggggcgtggcgcgggg |
| | ctcgccgtgccgggcggggggggcggcaggtgggggtgccgggcggggggggccg |
| | cctcgggccggggagggctcgggggaggggcgcggcggcccccggagcgccggcg |
| | gctgtcgaggcgcggcgagccgcagccattgccttttatggtaatcgtgcgagag |
| | ggcgcagggacttcctttgtcccaaatctgtgcggagccgaaatctgggaggcgc |
| | cgccgcaccccctctagcgggcgcggggcgaagcggtgcggcgccggcaggaagg |
| | aaatgggcggggagggccttcgtgcgtcgccgcgccgccgtccccttctccctct |
| | ccagcctcggggctgtccgcggggggacggctgccttcgggggggacggggcagg |
| | gcggggttcggcttctggcgtgtgaccggcggctctagagcctctgctaaccatg |
| | ttcatgccttcttctttttcctacagctcctgggcaacgtgctggttattgtgct |
| | gtctcatcattttggcaaagaattgcaagtttgtacaaaaaagcaggctGAATGC |
| | CGCCACCATGGCTaccgagtataaaccgacggtccgacttgcgactcgggatgac |
| | gttccacgcgccgtgaggactctggccgccgccttcgctgactacccggccacac |
| | ggcacacagttgaccccgacagacacatagagcgggtcacagagttgcaagaact |
| | tttcctgacgcgggttggActcgatattggaaaagtttgggttgccgacgatggt |
| | gccgcagttgcggtgtggactactccagaaagtgtagaagctggagccgtattcg |
| | ctgaaatagggccacggatggcagagctgtctggtagcaggctcgcagcgcagca |
| | gcagatggaaggCcttctcgctccgcatcggcctaaggaaccggcgtggttcctg |
| | gctacggtgggagtctcacctgaccatcaaggaaagggattgggaagtgctgtcg |
| | ttcttccaggcgtggaagcggccgaacgagcaggcgtcccagccttcttggaAac |
| | gagcgcacccaggaatcttccattctacgagcgactgggtttcactgtgacagca |
| | gacgtagaagtccctgaagggcctcggacttggtgcatgaccagaaagcctgggg |
| | cttgaAACGCCATgatccagacatgataagatacattgatgagtttggacaaacc |
| | acaactagaatgcagtgaGGCGCGCCTGGATACTTCTGCCTGCAGGGGAGTCGCt |
| | cttttgcaatgctctttgggaaattatattagcctaattactaatttcctggccc |
| | tcaaggtgacttgcttgaacttgccacctTCGCCACTAGGGGGCAGCAttggttt |
| | acacagggtgaacaaacgttcaattctaaactaatattctttggtgggaaagtgt |
| | gttcattttggtttgttttgaagaagtcacttcatacctttgaactggggttaag |
| | ctggttaactcccaaaattccaccagttccaatatcctatggaaaaaccccaaaa |
| | ccactCAAAggggttggggttgcgccttttccaaggcagccctgggtttgcgcag |
| | ggacgcggctgctctgggcgtggttccgggaaacgcagcggcgccgaccctgggT |
| | ctGgcacattcttcacgtccgttcgcagcgtcacccggatcttcgccgctaccct |
| | tgtgggccccccggcgacgcttcctgctccgcccctaagtcgggaaggttccttg |
| | cggttcgcggcgtgccggacgtgacaaacggaagccgcacgTctGactagtaccc |
| | tcgcagacggacagcgccagggagcaatggcagcgcgccgaccgcgatgggctgt |
| | ggccaatagcggctgctcagcAgggcgcgccgagagcagcggccgggaaggggcg |
| | gtgcgggaggcggggtgtggggcggtagtgtgggccctgttcctgcccgcgcggt |
| | gttccgcattctgcaagcctccggagcgcacgtcggcagtcggctccctcgttga |
| | ccgaatcaccgacctctctccccagGAATGCCGCCACCATGAATAACGCCGGCCA |
| | CGGACCAACTAGACGCTTGAGGGGACTCGGAGTTTTGGCTGGAGTCGCACTCCTG |
| | GCCGCTCTGTGGCTGCTTTGGCTCCTTGGAAGCGCGCCAAGGGGCACACCTGCAC |
| | CACAACCTACCATAACAATTCTGGTGTGGCATTGGCCATTTACCGATCAACCACC |
| | CGAATTGCCGTCAGATACATGTACCAGGTATGGGATTGCACGGTGTCATCTCAGC |
| | GCGAATCGCTCATTGTTGGCTTCAGCAGATGCGGTAGTGTTTCATCATAGAGAAC |
| | TTCAAACTCGCAGGAGCCACCTTCCTTTGGCACAAAGGCCGCGGGGTCAACCGTG |
| | GGTTTGGGCATCAATGGAAAGTCCCTCACATACTCATGGGCTGTCACATCTGCGG |
| | GGAATCTTTAATTGGGTACTTTCCTATCGACGGGATAGCGATATTTTCGTCCCAT |
| | ACGGACGATTGGAACCTCATTGGGGTCCTTCTCCCCCTCTCCCTGCAAAATCCAG |
| | AGTTGCAGCTTGGGTCGTTTCCAATTTTCAAGAACGCCAACTTAGGGCTCGCCTC |
| | TATAGACAACTCGCTCCCCACCTTCGTGTTGACGTTTTCGGTCGAGCTAACGGGC |
| | GCCCGTTGTGTGCGTCATGTCTCGTACCTACGGTTGCTCAATATCGGTTTTATCT |
| | CTCATTCGAAAATTCCCAACATCGGGATTATATAACTGAAAAGTTTTGGCGTAAT |
| | GCGTTGGTCGCAGGAACCGTACCCGTAGTCCTCGGACCACCCCGCGCAACTTACG |
| | AGGCTTTTGTTCCCGCAGACGCATTCGTACACGTGGACGATTTCGGTTCCGCTCG |
| | GGAACTCGCTGCATTTCTGACAGGTATGAACGAATCTAGGTATCAGCGGTTCTTC |
| | GCGTGGCGGGATCGGCTTCGGGTCAGGTTGTTTACGGATTGGAGAGAGAGATTTT |
| | GCGCAATATGCGATAGGTATCCTCATCTGCCTAGATCCCAGGTGTACGAAGATTT |
| | GGAAGGCTGGTTCCAAGCATAGAACGCCATgggtggcatccctgtgacccctccc |
| | cagtgcctctcctggccctggaagttgccactccagtgcccaccagccttgtcct |
| | aataaaattaagttgcatcattttgtctgactaggtgtccttctataatattatg |
| | gggtggaggggggtggtatggagcaaggggcaagttgggaagagaacctgtaggg |
| | cctgcggggtctattgggaaccaagctggagtgcagtggcacaatcttggctcac |
| | tgcaatctccgcctcctgggttcaagcgattctcctgcctcagcctcccgagttg |
| | ttgggattccaggcatgcatgaccaggctcagctaatttttgtttttttggtaga |
| | gTcggggtttcaccatattggccaggctggtgtccaactcctaatctcaggtgat |
| | ctacccaccttggcctcccaaattgctgggattacaggcgtgaaccactgctccc |
| | ttccctgtccttCAGTGGCATCGCtcttttgcaatgctctttgggaaattatatt |
| | agcctaattactaatttcctggccctcaaggtgacttgcttgaacttgccacctT |
| | CGCCACTAGGGGGCAGCAttggtttacacagggtgaacaaacgttcaattctaaa |
| | ctaatattctttggtgggaaagtgtgttcattttggtttgttttgaagaagtcac |
| | ttcatacctttgaactggggttaagctggttaactcccaaaattccaccagttcc |
| | aatatcctatggaaaaaccccaaaaccactCAAAggatctgcgatcgctccggtg |
| | cccgtcagtgggcagagcgcacatcgcccacagtccccgagaagttggggggagg |
| | ggtcggcaattgaacgggtgcctagagaaggtggcgcggggtaaactgggaaagt |
| | gatgtcgtgtactggctccgcctttttcccgaggggggggagaaccgtatataag |
| | tgcagtagtcgccgtgaacgttctttttcgcaacgggtttgccgccagaacacag |
| | ctgaagcttcgaggggctcgcatctctccttcacgcgcccgccgccctacctgag |
| | gccgccatccacgccggttgagtcgcgttctgccgcctcccgcctgtggtgcctc |
| | ctgaactgcgtccgccgtctaggtaagtttaaagctcaggtcgagTccgggcctt |
| | tgtccggcgctcccttggagcctacctagactcagccggctctccacgctttgcc |
| | tgaccctgcttgctcaactctacgtctttgtttcgttttctgttctgcgccgtta |
| | cagatccaagctgtgaccggcgcctacGAATGCCGCCACCATGCCCCTTCAGCTG |
| | TTGTTGCTTTTGATACTGCTGGGACCTGGAAACTCACTTCAGTTGTGGGACACAT |
| | GGGCCGATGAGGCGGAGAAGGCCCTGGGTCCCCTTCTCGCAAGAGACAGACGGCA |
| | AGCGACTGAGTATGAATACCTCGATTATGATTTCCTCCCCGAGACtGAGCCTCCT |
| | GAGATGTTGCGCAACAGTACAGACACAACGCCATTGACTGGCCCCGGAACTCCAG |
| | AGAGCACCACGGTTGAGCCTGCTGCCCGGAGAAGTACTGGATTGGATGCGGGCGG |
| | CGCTGTGACTGAATTGACCACTGAGCTGGCCAATATGGGGAATCTGTCCACAGAC |
| | AGCGCGGCCATGGAGATACAAACGACACAGCCTGCGGCCACCGAGGCCCAGACTA |
| | CACAGCCTGTACCGACAGAGGCCCAAACTACACCATTGGCAGCTACGGAAGCTCA |
| | aACaACTCGAttgACcGCtACCGAgGCACAGACTACCCCTCTCGCTGCGACtGAA |
| | GCcCAaACGACaCCACCTGCaGCaACaGAAGCACAAACTACACAGCCCACAGGGT |
| | TGGAGGCGCAAACAACTGCGCCTGCTGCCATGGAAGCTCAAACAACGGCACCGGC |
| | CGCAATGGAGGCaCAGACTACtCCtCCtGCaGCTATGGAGGCcCAAACGACaCAG |
| | ACGACTGCcATGGAGGCACAGACAACTGCACCAGAGGCCACGGAGGCACAAACAA |
| | CACAACCAACTGCAACAGAGGCGCAGACGACGCCCCTCGCAGCAATGGAAGCTCT |
| | TTCCACGGAACCCAGCGCAACCGAAGCACTCTCTATGGAGCCTACAACCAAACGG |
| | GGCCTTTTTATTCCATTCTCTGTGTCATCCGTGACTCATAAGGGGATACCTATGG |
| | CTGCGTCTAATCTCTCAGTCAATTATCCAGTTGGTGCGCCAGATCACATTTCCGT |
| | GAAACAGTGTCTCCTTGCCATATTGATACTTGCCTTGGTCGCGACTATTTTTTTC |
| | GTGTGTACCGTAGTGCTCGCGGTTCGCCTCTCTCGGAAGGGTCACATGTACCCTG |
| | TTAGGAATTATTCTCCCACTGAAATGGTGTGCATCAGCAGTCTGCTGCCTGATGG |
| | TGGCGAGGGACCATCCGCTACAGCCAACGGAGGACTCTCTAAGGCTAAGTCTCCT |
| | GGACTGACCCCTGAGCCCCGCGAGGATAGGGAAGGtGACGATCTCACCCTCCACT |
| | CCTTTCTGCCAtagAACGCCATgaattcactcctcaggtgcaggctgcctatcag |
| | aaggtggtggctggtgtggccaatgccctggctcacaaataccactgagatcttt |
| | ttccctctgccaaaaattatggggacatcatgaagccccttgagcatctgacttc |
| | tggctaataaaggaaatttattttcattgcaatagtgtgttggaattttttgtgt |
| | ctctcactcggaaggacatatgggagggcaaatcatttaaaacatcagaatgagt |
| | atttggtttagagtttggcaacatatgcccatatgctggctgccatgaacaaagg |
| | ttggctataaagaggtcatcagtatatgaaacagccccctgctgtccattcctta |
| | ttccatagaaaagccttgacttgaggttagattttttttatattttgttttgtgt |
| | tatttttttctttaacatccctaaaattttccttacatgttttactagccagatt |
| | tttcctcctctcctgactactcccagtcatagctgtccctcttctcttatggaga |
| | tCAGTTCGCTCGCtcttttgcaatgctctttgggaaattatattagcctaattac |
| | taatttcctggccctcaaggtgacttgcttgaacttgccacctTCGCCACTAGGG |
| | GGCAGCAttggtttacacagggtgaacaaacgttcaattctaaactaatattctt |
| | tggtgggaaagtgtgttcattttggtttgttttgaagaagtcacttcataccttt |
| | gaactggggttaagctggttaactcccaaaattccaccagttccaatatcctatg |
| | gaaaaaccccaaaaccactCAAAggatctgcgatcgctccggtgcccgtcagtgg |
| | gcagagcgcacatcgcccacagtccccgagaagttggggggaggggtcggcaatt |
| | gaacgggtgcctagagaaggtggcgcggggtaaactgggaaagtgatgtcgtgta |
| | ctggctccgcctttttcccgagggtgggggagaaccgtatataagtgcagtagtc |
| | gccgtgaacgttctttttcgcaacgggtttgccgccagaacacagctgaagcttc |
| | gaggggctcgcatctctccttcacgcgcccgccgccctacctgaggccgccatcc |
| | acgccggttgagtcgcgttctgccgcctcccgcctgtggtgcctcctgaactgcg |
| | tccgccgtctaggtaagtttaaagctcaggtcgagTccgggcctttgtccggcgc |
| | tcccttggagcctacctagactcagccggctctccacgctttgcctgaccctgct |
| | tgctcaactctacgtctttgtttcgttttctgttctgcgccgttacagatccaag |
| | ctgtgaccggcgcctacGAATGCCGCCACCATGGTGAGCAAATCTCGCTGGAAAC |
| | TGCTAGCCATGCTGGCTCTCGTCTTGGTCGTGATGGTATGGTATTCCATAAGTAG |
| | GGAGGACAGATACATAGAACTCTTCTACTTTCCCATTCCAGAGAAGAAGGAACCA |
| | TGTCTGCAGGGCGAGGCTGAGTCTAAAGCTTCAAAGCTGTTCGGGAACTATAGTC |
| | GAGATCAGCCTATTTTCCTGAGGCTCGAGGACTACTTCTGGGTGAAAACCCCCAG |
| | CGCATACGAACTGCCCTATGGGACCAAGGGATCAGAGGATCTCTTGCTCCGGGTG |
| | CTCGCCATCACATCAAGCAGCATTCCAAAAAACATCCAATCCTTGAGATGCCGTC |
| | GGTGTGTAGTGGTTGGCAACGGCCATAGGCTAAGAAATTCAAGCCTTGGTGATGC |
| | AATTAACAAATACGACGTGGTCATCCGCCTTAATAACGCCCCCGTGGCCGGATAT |
| | GAGGGCGATGTCGGTTCCAAAACGACTATGCGGCTTTTTTACCCTGAGTCTGCAC |
| | ATTTTGACCCTAAGGTGGAGAATAACCCTGATACCCTTCTGGTCCTGGTTGCCTT |
| | TAAGGCCATGGACTTCCACTGGATTGAAACCATTCTCAGCGACAAGAAACGCGTT |
| | CGGAAGGGGTTCTGGAAACAGCCGCCTCTGATATGGGATGTTAATCCCAAGCAAA |
| | TCAGGATCTTAAACCCCTTTTTCATGGAAATTGCCGCTGATAAGCTGCTGTCCTT |
| | ACCAATGCAGCAGCCGAGAAAGATCAAGCAGAAACCTACAACTGGCTTGTTAGCA |
| | ATAACTTTGGCTCTGCACCTGTGCGACCTTGTGCATATCGCAGGATTCGGGTACC |
| | CAGACGCGTATAATAAGAAGCAGACAATCCACTACTATGAACAGATCACCCTCAA |
| | ATCCATGGCTGGTAGTGGCCACAATGTGTCGCAAGAGGCCCTAGCCATTAAACGA |
| | ATGCTGGAAATGGGAGCGATCAAGAATCTGACATCTTTTTAAAACGCCATgggtg |
| | gcatccctgtgacccctccccagtgcctctcctggccctggaagttgccactcca |
| | gtgcccaccagccttgtcctaataaaattaagttgcatcattttgtctgactagg |
| | tgtccttctataatattatggggtggaggggggtggtatggagcaaggggcaagt |
| | tgggaagagaacctgtagggcctgcggggtctattgggaaccaagctggagtgca |
| | gtggcacaatcttggctcactgcaatctccgcctcctgggttcaagcgattctcc |
| | tgcctcagcctcccgagttgttgggattccaggcatgcatgaccaggctcagcta |
| | atttttgtttttttggtagagTcggggtttcaccatattggccaggctggtgtcc |
| | aactcctaatctcaggtgatctacccaccttggcctcccaaattgctgggattac |
| | aggcgtgaaccactgctcccttccctgtccttctgattttaaaataactatacca |
| | gcaggaggacgtccagacacagcataggctacctggccatgcccaaccggtggga |
| | catttgagttgcttgcttggcactgtcctctcatgcgttgggtccactcagtaga |
| | tgcctgttCAGTCAGTTCGCtcttttgcaatgctctttgggaaattatattagcc |
| | taattactaatttcctggccctcaaggtgacttgcttgaacttgccacctTCGCC |
| | ACTAGGGGGCAGCAttggtttacacagggtgaacaaacgttcaattctaaactaa |
| | tattctttggtgggaaagtgtgttcattttggtttgttttgaagaagtcacttca |
| | tacctttgaactggggttaagctggttaactcccaaaattccaccagttccaata |
| | tcctatggaaaaaccccaaaaccactCAAAggatctgcCatcgctccggtgcccg |
| | tcagtgggcagagcgcacatcgcccacagtccccgagaagttggggggaggggtc |
| | ggcaattgaacgggtgcctagagaaggtggcgcggggtaaactgggaaagtgatg |
| | tcgtgtactggctccgcctttttcccgaggggggggagaaccgtatataagtgca |
| | gtagtcgccgtgaacgttctttttcgcaacgggtttgccgccagaacacagctga |
| | agcttcgaggggctcgcatctctccttcacgcgcccgccgccctacctgaggccg |
| | ccatccacgccggttgagtcgcgttctgccgcctcccgcctgtggtgcctcctga |
| | actgcgtccgccgtctaggtaagtttaaagctcaggtcgagTccgggcctttgtc |
| | cggcgctcccttggagcctacctagactcagccggctctccacgctttgcctgac |
| | cctgcttgctcaactctacgtctttgtttcgttttctgttctgcgccgttacaga |
| | tccaagctgtgaccggcgcctacGAATGCCGCCACCATGGCTgctgaaggTtccg |
| | tcgccaggcagcctgacctcttgacctgcgacgatgagccgatccatatccccgg |
| | tgccatccaaccgcatggactgctgctcgccctcgccgccgacatgacgatcgtt |
| | gccggcagcgacaaccttcccgaactcaccggactggcgatcggcgccctgatcg |
| | gccgctctgcggccgatgtGttcgactcggaAacgcacaaccgtctgacgatcgc |
| | cttggccgagcccggggcggccgtcggagcaccgatcactgtcggcttcacgatg |
| | cgaaaggacgcaggcttcatcggctcctggcatcgccatgatcagctcatcttcc |
| | tGgagctGgagcctccccagcgggacgtcgccgagccgcaggcgttcttccgccg |
| | caccaacagcgccatccgccgcctgcaggccgccgaaaccttggaaagcgcctgc |
| | gccgccgcggcgcaagaggtgcggaagattaccggcttcgatcgggtgatgatct |
| | atcgcttcgcctccgacttcagcggcgaagtgatcgcagaggatcggtgcgccga |
| | ggtcgagtcaaaactaggcctgcactatcctgcctcaaccgtgccggcgcaggcc |
| | cgtcggctctataccatcaacccggtacggatcattcccgatatcaattatcggc |
| | cggtgccggtcaccccagacctcaatccggtcaccgggcggccgattgatcttag |
| | cttcgccatcctgcgcagcgtGtcgcccgtccatctggaGttcatgcgcaacata |
| | ggcatgcacggcacgatgtcgatctcgattttgcgcggcgagcgactgtggggat |
| | tgatcgtttgccatcaccgaacgccgtactacgtcgatctcgatggccgccaagc |
| | ctgcgagctagtcgcccaggttctggcctggcagatcggcgtgatggaagagTAA |
| | AACGBBABBABBABBABBABBACCATctgtgccttctagttgccagccatctgtt |
| | gtttgcccctcccccgtgccttccttgaccctggaaggtgccactcccactgtcc |
| | tttcctaataaaatgaggaaattgcatcgcattgtctgagtaggtgtcattctat |
| | tctggggggtggggggggcaggacagcaagggggaggattgggaaggcaatagca |
| | ggcatgctggggatgcggtgggctctatggCAGTTAGGGCGGCCGCGCCTGGGCT |
| | TACttaattaacgagagcataatattgatatgtgccaaagttgtttctgactgac |
| | taataagtataatttgtttctattatgtataggttaagctaattacttattttat |
| | aatacaacatgactgtttttaaagtacaaaataagtttatttttgtaaaagagag |
| | aatgtttaaaagttttgttactttatagaagaaattttgagtttttgtttttttt |
| | taataaataaataaacataaataaattgtttgttgaatttattattagtatgtaa |
| | gtgtaaatataataaaacttaatatctattcaaattaataaataaacctcgatat |
| | acagaccgataaaacacatgcgtcaattttacgcatgattatctttaacgtacgt |
| | cacaatatgattatctttctagggttaaataatagtttctaatttttttattatt |
| | cagcctgctgtcgtgaataccgagctccaattcgccctatagtgagtcgtattac |
| | aattcactggccgtcgttttacaacgtcgtgactgggaaaaccctggcgttaccc |
| | aacttaatcgccttgcagcacatccccctttcgccagctggcgtaatagcgaaga |
| | ggcccgcaccgatcgcccttcccaacagttgcgcagcctgaatggcgaatgggac |
| | gcgccctgtagcggcgcattaagcgcggcgggtgtggtggttacgcgcagcgtga |
| | ccgctacacttgccagcgccctagcgcccgctcctttcgctttcttcccttcctt |
| | tctcgccacgttcgccggctttccccgtcaagctctaaatcgggggctcccttta |
| | gggttccgatttagtgctttacggcacctcgaccccaaaaaacttgattagggtg |
| | atggttcacgtagtgggccatcgccctgatagacggtttttcgccctttgacgtt |
| | ggagtccacgttctttaatagtggactcttgttccaaactggaacaacactcaac |
| | cctatctcggtctattcttttgatttataagggattttgccgatttcggcctatt |
| | ggttaaaaaatgagctgatttaacaaaaatttaacgcgaattttaacaaaatatt |
| | aacgcttacaatttaggtggcacttttcggggaaatgtgcgcggaacccctattt |
| | gtttatttttctaaatacattcaaatatgtatccgctcatgagacaataaccctg |
| | ataaatgcttcaataatattgaaaaaggaagagtatgagtattcaacatttccgt |
| | gtcgcccttattcccttttttgcggcattttgccttcctgtttttgctcacccag |
| | aaacgctggtgaaagtaaaagatgctgaagatcagttggg |
| | (SEQ ID NO: 38) |
| pAJW_P112 (LFA-1 | tgcacgagtgggttacatcgaactggatctcaacagcggtaagatccttgagagt |
| integrin pair | tttcgccccgaagaacgttttccaatgatgagcacttttaaagttctgctatgtg |
| (Integrin αL | gcgcggtattatcccgtattgacgccgggcaagagcaactcggtcgccgcataca |
| and | ctattctcagaatgacttggttgagtactcaccagtcacagaaaagcatcttacg |
| Integrin β2)) | gatggcatgacagtaagagaattatgcagtgctgccataaccatgagtgataaca |
| | ctgcggccaacttacttctgacaacgatcggaggaccgaaggagctaaccgcttt |
| | tttgcacaacatgggggatcatgtaactcgccttgatcgttgggaaccggagctg |
| | aatgaagccataccaaacgacgagcgtgacaccacgatgcctgtagcaatggcaa |
| | caacgttgcgcaaactattaactggcgaactacttactctagcttcccggcaaca |
| | attaatagactggatggaggcggataaagttgcaggaccacttctgcgctcggcc |
| | cttccggctggctggtttattgctgataaatctggagccggtgagcgtgggtctc |
| | gcggtatcattgcagcactggggccagatggtaagccctcccgtatcgtagttat |
| | ctacacgacggggagtcaggcaactatggatgaacgaaatagacagatcgctgag |
| | ataggtgcctcactgattaagcattggtaactgtcagaccaagtttactcatata |
| | tactttagattgatttaaaacttcatttttaatttaaaaggatctaggtgaagat |
| | cctttttgataatctcatgaccaaaatcccttaacgtgagttttcgttccactga |
| | gcgtcagaccccgtagaaaagatcaaaggatcttcttgagatcctttttttctgc |
| | gcgtaatctgctgcttgcaaacaaaaaaaccaccgctaccagcggtggtttgttt |
| | gccggatcaagagctaccaactctttttccgaaggtaactggcttcagcagagcg |
| | cagataccaaatactgttcttctagtgtagccgtagttaggccaccacttcaaga |
| | actctgtagcaccgcctacatacctcgctctgctaatcctgttaccagtggctgc |
| | tgccagtggcgataagtcgtgtcttaccgggttggactcaagacgatagttaccg |
| | gataaggcgcagcggtcgggctgaacggggggttcgtgcacacagcccagcttgg |
| | agcgaacgacctacaccgaactgagatacctacagcgtgagctatgagaaagcgc |
| | cacgcttcccgaagggagaaaggcggacaggtatccggtaagcggcagggtcgga |
| | acaggagagcgcacgagggagcttccagggggaaacgcctggtatctttatagtc |
| | ctgtcgggtttcgccacctctgacttgagcgtcgatttttgtgatgctcgtcagg |
| | ggggcggagcctatggaaaaacgccagcaacgcggcctttttacggttcctggcc |
| | ttttgctggccttttgctcacatgttctttcctgcgttatcccctgattctgtgg |
| | ataaccgtattaccgcctttgagtgagctgataccgctcgccgcagccgaacgac |
| | cgagcgcagcgagtcagtgagcgaggaagcggaagagcgcccaatacgcaaaccg |
| | cctctccccgcgcgttggccgattcattaatgcagctggcacgacaggtttcccg |
| | actggaaagcgggcagtgagcgcaacgcaattaatgtgagttagctcactcatta |
| | ggcaccccaggctttacactttatgcttccggctcgtatgttgtgtggaattgtg |
| | agcggataacaatttcacacaggaaacagctatgaccatgattacgccaagctcg |
| | aaattaaccctcactaaagggaacaaaagctggtacctcgcgcgacttggtttgc |
| | cattctttagcgcgcgtcgcgtcacacagcttggccacaatgtggtttttgtcaa |
| | acgaagattctatgacgtgtttaaagtttaggtcgagtaaagcgcaaatcttttt |
| | taaccctagaaagatagtctgcgtaaaattgacgcatgcattcttgaaatattgc |
| | tctctctttctaaatagcgcgaatccgtcgctgtgcatttaggacatctcagtcg |
| | ccgcttggagctcccgtgaggcgtgcttgtcaatgcggtaagtgtcactgatttt |
| | gaactataacgaccgcgtgagtcaaaatgacgcatgattatcttttacgtgactt |
| | ttaagatttaactcatacgataattatattgttatttcatgttctacttacgtga |
| | taacttattatatatatattttcttgttatagatatcatcaactttgtatagaaa |
| | agttgctcgacattgattattgactagttattaatagtaatcaattacggggtca |
| | ttagttcatagcccatatatggagttccgcgttacataacttacggtaaatggcc |
| | cgcctggctgaccgcccaacgacccccgcccattgacgtcaataatgacgtatgt |
| | tcccatagtaacgccaatagggactttccattgacgtcaatgggtggagtattta |
| | cggtaaactgcccacttggcagtacatcaagtgtatcatatgccaagtacgcccc |
| | ctattgacgtcaatgacggtaaatggcccgcctggcattatgcccagtacatgac |
| | cttatgggactttcctacttggcagtacatctacgtattagtcatcgctattacc |
| | atggtcgaggtgagccccacgttctgcttcactctccccatctcccccccctccc |
| | cacccccaattttgtatttatttattttttaattattttgtgcagcgatgggggc |
| | ggggggggggggggggcgcgcgccaggcggggggggcggggcgaggggcggggcg |
| | gggcgaggcggagaggtgcggcggcagccaatcagagcggcgcgctccgaaagtt |
| | tccttttatggcgaggcggcggcggcggcggccctataaaaagcgaagcgcgcgg |
| | cgggcgggagtcgctgcgcgctgccttcgccccgtgccccgctccgccgccgcct |
| | cgcgccgcccgccccggctctgactgaccgcgttactcccacaggtgagcgggcg |
| | ggacggcccttctcctccgggctgtaattagcgcttggtttaatgacggcttgtt |
| | tcttttctgtggctgcgtgaaagccttgaggggctccgggagggccctttgtgcg |
| | gggggagcggctcggggggtgcgtgcgtgtgtgtgtgcgtggggagcgccgcgtg |
| | cggctccgcgctgcccggcggctgtgagcgctgcgggcgcggcgcggggctttgt |
| | gcgctccgcagtgtgcgcgaggggagcgcggccgggggcggtgccccgcggtgcg |
| | gggggggctgcgaggggaacaaaggctgcgtgcggggtgtgtgcgtgggggggtg |
| | agcagggggtgtgggcgcgtcggtcgggctgcaaccccccctgcacccccctccc |
| | cgagttgctgagcacggcccggcttcgggtgcggggctccgtacggggcgtggcg |
| | cggggctcgccgtgccgggcggggggggcggcaggtgggggtgccgggcgggggg |
| | ggccgcctcgggccggggagggctcgggggaggggcgcggcggcccccggagcgc |
| | cggcggctgtcgaggcgcggcgagccgcagccattgccttttatggtaatcgtgc |
| | gagagggcgcagggacttcctttgtcccaaatctgtgcggagccgaaatctggga |
| | ggcgccgccgcaccccctctagcgggcgcggggcgaagcggtgcggcgccggcag |
| | gaaggaaatgggcggggagggccttcgtgcgtcgccgcgccgccgtccccttctc |
| | cctctccagcctcggggctgtccgcggggggacggctgccttcgggggggacggg |
| | gcagggcggggttcggcttctggcgtgtgaccggcggctctagagcctctgctaa |
| | ccatgttcatgccttcttctttttcctacagctcctgggcaacgtgctggttatt |
| | gtgctgtctcatcattttggcaaagaattgcaagtttgtacaaaaaagcaggctG |
| | AATGCCGCCACCATGGCTaccgagtataaaccgacggtccgacttgcgactcggg |
| | atgacgttccacgcgccgtgaggactctggccgccgccttcgctgactacccggc |
| | cacacggcacacagttgaccccgacagacacatagagcgggtcacagagttgcaa |
| | gaacttttcctgacgcgggttggActcgatattggaaaagtttgggttgccgacg |
| | atggtgccgcagttgcggtgtggactactccagaaagtgtagaagctggagccgt |
| | attcgctgaaatagggccacggatggcagagctgtctggtagcaggctcgcagcg |
| | cagcagcagatggaaggCcttctcgctccgcatcggcctaaggaaccggcgtggt |
| | tcctggctacggtgggagtctcacctgaccatcaaggaaagggattgggaagtgc |
| | tgtcgttcttccaggcgtggaagcggccgaacgagcaggcgtcccagccttcttg |
| | gaAacgagcgcacccaggaatcttccattctacgagcgactgggtttcactgtga |
| | cagcagacgtagaagtccctgaagggcctcggacttggtgcatgaccagaaagcc |
| | tggggcttgaAACGCCATgatccagacatgataagatacattgatgagtttggac |
| | aaaccacaactagaatgcagtgaGGCGCGCCTGGATACTTCTGCCTGCAGGGGAG |
| | TCGCtcttttgcaatgctctttgggaaattatattagcctaattactaatttcct |
| | ggccctcaaggtgacttgcttgaacttgccacctTCGCCACTAGGGGGCAGCAtt |
| | ggtttacacagggtgaacaaacgttcaattctaaactaatattctttggtgggaa |
| | agtgtgttcattttggtttgttttgaagaagtcacttcatacctttgaactgggg |
| | ttaagctggttaactcccaaaattccaccagttccaatatcctatggaaaaaccc |
| | caaaaccactCAAAggatctgcgatcgctccggtgcccgtcagtgggcagagcgc |
| | acatcgcccacagtccccgagaagttggggggaggggtcggcaattgaacgggtg |
| | cctagagaaggtggcgcggggtaaactgggaaagtgatgtcgtgtactggctccg |
| | cctttttcccgaggggggggagaaccgtatataagtgcagtagtcgccgtgaacg |
| | ttctttttcgcaacgggtttgccgccagaacacagctgaagcttcgaggggctcg |
| | catctctccttcacgcgcccgccgccctacctgaggccgccatccacgccggttg |
| | agtcgcgttctgccgcctcccgcctgtggtgcctcctgaactgcgtccgccgtct |
| | aggtaagtttaaagctcaggtcgagTccgggcctttgtccggcgctcccttggag |
| | cctacctagactcagccggctctccacgctttgcctgaccctgcttgctcaactc |
| | tacgtctttgtttcgttttctgttctgcgccgttacagatccaagctgtgaccgg |
| | cgcctacGAATGCCGCCACCATGAAAGATAGCTGTATCACTGTCATGGCCATGGC |
| | CCTGTTGAGCGGCTTCTTCTTTTTTGCTCCAGCATCCTCCTATAACCTGGACGTT |
| | CGCGGGGCACGGTCCTTTTCCCCTCCTAGGGCCGGCAGGCATTTTGGCTACCGTG |
| | TGCTGCAGGTCGGTAACGGAGTAATCGTGGGTGCCCCGGGAGAAGGAAACAGCAC |
| | GGGAAGTCTGTACCAGTGCCAGTCTGGTACCGGCCACTGCTTACCCGTCACCCTA |
| | CGCGGGAGTAACTACACATCCAAATACCTCGGAATGACTCTTGCAACAGATCCGA |
| | CCGATGGGAGCATTTTGGCCTGTGATCCAGGCCTGTCTCGAACCTGCGACCAAAA |
| | CACATACCTGTCCGGACTCTGTTACCTCTTTCGCCAGAATTTGCAGGGGCCGATG |
| | CTGCAGGGCCGGCCCGGATTTCAGGAGTGTATAAAAGGCAATGTGGACCTAGTAT |
| | TTCTTTTTGACGGAAGCATGAGCCTGCAACCGGATGAGTTTCAGAAGATCCTAGA |
| | CTTTATGAAAGATGTTATGAAGAAGCTCTCCAACACTTCCTATCAGTTTGCCGCA |
| | GTGCAGTTCTCCACCTCCTATAAGACGGAGTTTGATTTCTCTGACTATGTGAAAC |
| | GCAAAGATCCCGATGCCCTGCTGAAGCATGTGAAGCATATGTTGTTGCTGACTAA |
| | CACCTTTGGGGCTATAAACTATGTGGCCACCGAGGTGTTTAGGGAGGAATTGGGT |
| | GCCCGCCCTGACGCCACCAAGGTCCTTATTATCATAACAGACGGCGAAGCCACAG |
| | ACTCAGGCAACATTGATGCTGCAAAAGATATAATTCGATACATTATAGGAATCGG |
| | CAAGCACTTTCAGACAAAGGAGTCTCAAGAGACTCTCCATAAGTTTGCCAGTAAG |
| | CCTGCTTCAGAGTTCGTAAAAATTCTGGACACTTTCGAAAAGCTGAAAGACCTCT |
| | TTACAGAGCTCCAGAAGAAAATCTATGTCATCGAAGGCACTTCCAAGCAGGATCT |
| | GACTAGCTTCAACATGGAGCTCTCTTCAAGCGGCATTTCAGCTGATCTCTCTCGC |
| | GGTCACGCGGTCGTGGGGGCGGTAGGAGCCAAAGATTGGGCTGGTGGCTTCTTGG |
| | ACCTGAAGGCTGACCTGCAAGACGACACATTCATCGGTAATGAACCCCTAACGCC |
| | CGAAGTGAGAGCCGGGTACTTGGGCTATACTGTCACCTGGTTGCCATCGAGACAG |
| | AAAACCTCTCTGTTAGCCAGCGGAGCGCCCAGATACCAACACATGGGTCGGGTTC |
| | TCTTGTTTCAGGAACCACAAGGCGGAGGTCACTGGAGCCAGGTGCAGACTATCCA |
| | TGGAACCCAGATCGGAAGCTACTTCGGCGGGGAATTATGTGGCGTTGACGTGGAC |
| | CAGGACGGGGAGACTGAACTGCTTCTGATCGGAGCACCCCTTTTCTATGGAGAGC |
| | AGCGGGGAGGAAGAGTGTTCATCTACCAACGTAGACAGCTGGGTTTCGAAGAGGT |
| | TTCAGAGCTTCAAGGCGATCCAGGCTATCCCCTGGGGCGTTTTGGCGAGGCGATC |
| | ACGGCTCTGACCGATATTAACGGCGACGGCCTGGTGGACGTTGCAGTTGGGGCTC |
| | CGCTGGAGGAACAGGGTGCCGTATACATATTCAACGGCAGACACGGAGGACTTTC |
| | TCCCCAGCCCTCCCAGAGAATCGAGGGAACACAGGTCCTGTCTGGCATTCAGTGG |
| | TTCGGCCGGAGTATCCACGGAGTCAAGGATCTTGAAGGCGATGGGCTTGCAGACG |
| | TAGCCGTGGGGGCAGAAAGCCAGATGATCGTTTTGTCGTCAAGGCCTGTCGTTGA |
| | CATGGTCACTCTGATGTCCTTTTCCCCCGCCGAAATACCAGTACACGAGGTGGAG |
| | TGCTCCTATTCGACTAGTAATAAGATGAAAGAGGGTGTAAATATCACGATCTGCT |
| | TTCAGATTAAGTCTCTTATTCCACAGTTCCAGGGGCGCCTGGTGGCCAATCTCAC |
| | CTATACCTTACAGCTTGACGGGCACCGTACACGGCGGAGGGGTCTATTTCCAGGC |
| | GGGAGACACGAGCTCAGGAGGAATATAGCAGTGACAACAAGCATGAGTTGCACCG |
| | ACTTCAGCTTCCACTTCCCTGTCTGCGTCCAGGATCTGATTAGTCCTATCAATGT |
| | CTCTCTGAACTTCAGTCTTTGGGAGGAGGAAGGCACACCTAGAGATCAAAGGGCT |
| | CAGGGGAAGGATATACCTCCAATTTTGCGACCCTCCCTCCACAGCGAAACATGGG |
| | AAATCCCATTCGAAAAGAACTGTGGCGAAGATAAGAAATGTGAGGCTAATCTGCG |
| | AGTTTCATTTAGCCCCGCCAGGAGCCGCGCTCTGAGGCTTACCGCCTTCGCATCA |
| | TTAAGTGTGGAACTATCCCTCAGCAATCTCGAGGAGGACGCCTACTGGGTGCAAC |
| | TGGATCTCCATTTCCCTCCCGGACTCTCGTTTCGAAAGGTGGAAATGCTCAAGCC |
| | TCATAGTCAAATTCCAGTAAGCTGTGAGGAGCTCCCGGAGGAGTCTCGCCTGTTG |
| | TCTCGAGCATTAAGCTGCAATGTTAGCTCACCCATCTTCAAAGCCGGCCATTCAG |
| | TCGCACTGCAGATGATGTTCAACACCCTCGTGAACTCCAGCTGGGGTGACTCCGT |
| | CGAATTACATGCAAATGTTACCTGCAATAACGAGGATAGCGATCTCCTCGAGGAC |
| | AACTCCGCTACGACCATTATCCCTATTTTGTATCCCATCAATATTCTCATCCAGG |
| | ACCAAGAGGATTCAACACTGTACGTCAGCTTCACCCCAAAGGGTCCAAAGATTCA |
| | CCAGGTCAAACATATGTATCAGGTGCGGATTCAACCTTCAATACACGACCACAAT |
| | ATACCAACATTGGAGGCCGTAGTGGGGGTGCCACAGCCCCCTTCTGAGGGACCTA |
| | TCACCCACCAGTGGTCCGTGCAGATGGAGCCACCCGTTCCATGCCATTATGAAGA |
| | TCTGGAGCGGCTACCTGACGCTGCCGAACCTTGTTTGCCTGGGGCGCTGTTTAGA |
| | TGCCCCGTGGTGTTCCGGCAAGAAATTCTGGTGCAAGTGATCGGCACTTTAGAGC |
| | TTGTGGGGGAGATTGAAGCGTCTAGTATGTTCTCTCTGTGTAGTTCTCTGAGCAT |
| | CTCATTCAATAGTTCTAAGCATTTTCACCTTTACGGGTCAAATGCGTCACTGGCT |
| | CAAGTTGTGATGAAAGTGGACGTCGTGTACGAAAAACAGATGTTATACCTGTACG |
| | TGCTGAGTGGTATCGGCGGACTGCTACTGCTGCTGCTTATTTTCATCGTGCTGTA |
| | TAAGGTGGGATTCTTTAAAAGGAACTTAAAGGAAAAAATGGAAGCCGGGAGAGGG |
| | GTTCCGAACGGGATTCCCGCTGAGGACAGTGAGCAGCTCGCATCTGGTCAGGAGG |
| | CTGGGGACCCAGGGTGCCTCAAACCTCTGCATGAAAAAGATTCGGAGTCCGGCGG |
| | CGGCAAGGATTAAAACGCCATgggtggcatccctgtgacccctccccagtgcctc |
| | tcctggccctggaagttgccactccagtgcccaccagccttgtcctaataaaatt |
| | aagttgcatcattttgtctgactaggtgtccttctataatattatggggtggagg |
| | ggggtggtatggagcaaggggcaagttgggaagagaacctgtagggcctgcgggg |
| | tctattgggaaccaagctggagtgcagtggcacaatcttggctcactgcaatctc |
| | cgcctcctgggttcaagcgattctcctgcctcagcctcccgagttgttgggattc |
| | caggcatgcatgaccaggctcagctaatttttgtttttttggtagagTcggggtt |
| | tcaccatattggccaggctggtgtccaactcctaatctcaggtgatctacccacc |
| | ttggcctcccaaattgctgggattacaggcgtgaaccactgctcccttccctgtc |
| | cttCAGTGGCATCGCtcttttgcaatgctctttgggaaattatattagcctaatt |
| | actaatttcctggccctcaaggtgacttgcttgaacttgccacctTCGCCACTAG |
| | GGGGCAGCAttggtttacacagggtgaacaaacgttcaattctaaactaatattc |
| | tttggtgggaaagtgtgttcattttggtttgttttgaagaagtcacttcatacct |
| | ttgaactggggttaagctggttaactcccaaaattccaccagttccaatatccta |
| | tggaaaaaccccaaaaccactCAAAggatctgcgatcgctccggtgcccgtcagt |
| | gggcagagcgcacatcgcccacagtccccgagaagttggggggaggggtcggcaa |
| | ttgaacgggtgcctagagaaggtggcgcggggtaaactgggaaagtgatgtcgtg |
| | tactggctccgcctttttcccgagggtgggggagaaccgtatataagtgcagtag |
| | tcgccgtgaacgttctttttcgcaacgggtttgccgccagaacacagctgaagct |
| | tcgaggggctcgcatctctccttcacgcgcccgccgccctacctgaggccgccat |
| | ccacgccggttgagtcgcgttctgccgcctcccgcctgtggtgcctcctgaactg |
| | cgtccgccgtctaggtaagtttaaagctcaggtcgagTccgggcctttgtccggc |
| | gctcccttggagcctacctagactcagccggctctccacgctttgcctgaccctg |
| | cttgctcaactctacgtctttgtttcgttttctgttctgcgccgttacagatcca |
| | agctgtgaccggcgcctacGAATGCCGCCACCATGTTAGGTCTGCGCCCTCCACT |
| | GCTGGCCCTGGTAGGATTACTCTCCTTGGGGTGTGTTCTCAGCCAGGAGTGCACC |
| | AAATTTAAAGTCTCCAGCTGCCGGGAGTGTATCGAGAGCGGCCCAGGCTGCACAT |
| | GGTGCCAAAAACTCAACTTCACCGGACCAGGAGATCCCGACAGTATAAGATGTGA |
| | TACTAGGCCTCAGCTGTTGATGCGGGGCTGTGCTGCCGACGACATTATGGACCCT |
| | ACCAGCTTAGCAGAAACCCAGGAAGATCATAATGGGGGGCAAAAACAGCTCAGCC |
| | CCCAGAAAGTGACCCTGTACCTGCGACCCGGTCAAGCGGCTGCCTTCAACGTGAC |
| | TTTTCGGCGCGCGAAGGGATACCCCATCGATCTTTATTATCTGATGGATTTGAGC |
| | TACTCAATGCTGGATGACCTGAGGAACGTAAAGAAGTTGGGAGGGGATCTGCTGC |
| | GCGCTCTTAATGAAATTACAGAATCAGGCAGGATCGGCTTCGGATCATTCGTGGA |
| | CAAAACAGTGCTGCCGTTCGTGAACACACACCCGGACAAGCTAAGAAATCCTTGC |
| | CCCAACAAGGAAAAGGAGTGCCAGCCGCCATTTGCTTTCCGACACGTGCTGAAAC |
| | TCACGAATAACTCAAATCAATTCCAAACCGAAGTGGGCAAGCAGCTGATCTCTGG |
| | CAACCTGGACGCCCCCGAGGGGGGACTAGATGCGATGATGCAAGTTGCCGCTTGC |
| | CCCGAAGAGATTGGCTGGCGCAACGTGACCAGACTGCTGGTCTTTGCAACTGATG |
| | ACGGCTTCCATTTTGCTGGCGATGGAAAGTTGGGGGCAATCCTGACACCTAATGA |
| | CGGCCGCTGCCATCTAGAAGATAACCTCTATAAGAGGAGTAATGAGTTTGACTAT |
| | CCAAGTGTCGGCCAGCTGGCACATAAACTAGCCGAGAACAACATCCAACCGATCT |
| | TCGCAGTCACAAGTAGGATGGTAAAGACGTACGAGAAGCTGACTGAGATCATACC |
| | AAAATCTGCAGTGGGGGAGCTGAGCGAAGATTCTAGCAATGTTGTGCAGCTTATT |
| | AAGAATGCCTATAATAAGCTTTCCAGTAGAGTGTTTCTGGACCACAATGCCCTTC |
| | CCGACACTCTGAAAGTTACTTACGATTCTTTTTGTTCCAACGGCGTGACGCACCG |
| | GAATCAGCCTCGCGGTGACTGTGACGGCGTGCAGATCAACGTGCCTATTACATTT |
| | CAGGTTAAAGTCACTGCCACCGAGTGCATCCAGGAGCAATCATTTGTCATCAGGG |
| | CTCTCGGTTTCACGGACATTGTGACCGTTCAGGTGCTCCCTCAGTGCGAATGTCG |
| | ATGCAGAGATCAGAGCAGAGATAGGTCCTTATGTCACGGGAAGGGTTTCTTGGAA |
| | TGTGGGATATGCAGATGCGATACAGGCTATATAGGGAAGAACTGCGAATGCCAGA |
| | CACAGGGCCGATCGTCCCAGGAGCTGGAGGGGAGCTGTCGCAAAGATAACAACTC |
| | TATAATTTGCAGCGGGCTCGGTGACTGTGTGTGTGGTCAGTGTCTCTGCCACACT |
| | AGTGACGTACCAGGGAAACTCATTTATGGCCAGTACTGCGAGTGTGATACCATTA |
| | ATTGTGAGAGGTATAACGGACAGGTCTGCGGCGGGCCAGGCCGTGGACTTTGTTT |
| | CTGCGGTAAGTGCCGATGCCATCCTGGATTTGAAGGCAGTGCTTGTCAATGCGAG |
| | CGTACCACTGAGGGCTGTTTAAACCCTCGGCGTGTCGAGTGCTCGGGACGCGGTA |
| | GATGCCGGTGTAACGTCTGCGAGTGCCACTCTGGTTACCAGCTCCCCCTCTGTCA |
| | GGAATGTCCCGGATGCCCTTCTCCTTGCGGGAAGTATATTTCCTGTGCAGAATGT |
| | CTGAAGTTTGAGAAGGGCCCCTTCGGGAAGAACTGCTCCGCCGCGTGCCCAGGAC |
| | TTCAGTTATCAAATAATCCAGTAAAAGGGAGAACCTGTAAGGAAAGGGATTCCGA |
| | AGGCTGTTGGGTGGCTTACACACTGGAGCAGCAAGACGGTATGGACAGATACCTT |
| | ATCTACGTCGACGAGTCTCGGGAGTGTGTGGCCGGACCAAATATCGCTGCCATCG |
| | TCGGCGGAACAGTGGCCGGAATTGTTCTGATCGGGATACTTCTACTCGTTATCTG |
| | GAAAGCATTGATTCACTTGTCTGACCTGCGTGAATACCGGCGGTTCGAGAAAGAA |
| | AAGTTGAAATCGCAGTGGAATAATGACAATCCCCTCTTTAAATCAGCCACCACTA |
| | CCGTGATGAACCCCAAGTTCGCCGAGTCCTAAAACGCCATgaattcactcctcag |
| | gtgcaggctgcctatcagaaggtggtggctggtgtggccaatgccctggctcaca |
| | aataccactgagatctttttccctctgccaaaaattatggggggggacatcatga |
| | agccccttgagcatctgacttctggctaataaaggaaatttattttcattgcaat |
| | agtgtgttggaattttttgtgtctctcactcggaaggacatatgggagggcaaat |
| | catttaaaacatcagaatgagtatttggtttagagtttggcaacatatgcccata |
| | tgctggctgccatgaacaaaggttggctataaagaggtcatcagtatatgaaaca |
| | gccccctgctgtccattccttattccatagaaaagccttgacttgaggttagatt |
| | ttttttatattttgttttgtgttatttttttctttaacatccctaaaattttcct |
| | tacatgttttactagccagatttttcctcctctcctgactactcccagtcatagc |
| | tgtccctcttctcttatggagatCAGTTCGCTCGCtcttttgcaatgctctttgg |
| | gaaattatattagcctaattactaatttcctggccctcaaggtgacttgcttgaa |
| | cttgccacctTCGCCACTAGGGGGCAGCAttggtttacacagggtgaacaaacgt |
| | tcaattctaaactaatattctttggtgggaaagtgtgttcattttggtttgtttt |
| | gaagaagtcacttcatacctttgaactggggttaagctggttaactcccaaaatt |
| | ccaccagttccaatatcctatggaaaaaccccaaaaccactCAAAggatctgcCa |
| | tcgctccggtgcccgtcagtgggcagagcgcacatcgcccacagtccccgagaag |
| | ttggggggaggggtcggcaattgaacgggtgcctagagaaggtggcgcggggtaa |
| | actgggaaagtgatgtcgtgtactggctccgcctttttcccgagggtgggggaga |
| | accgtatataagtgcagtagtcgccgtgaacgttctttttcgcaacgggtttgcc |
| | gccagaacacagctgaagcttcgaggggctcgcatctctccttcacgcgcccgcc |
| | gccctacctgaggccgccatccacgccggttgagtcgcgttctgccgcctcccgc |
| | ctgtggtgcctcctgaactgcgtccgccgtctaggtaagtttaaagctcaggtcg |
| | agTccgggcctttgtccggcgctcccttggagcctacctagactcagccggctct |
| | ccacgctttgcctgaccctgcttgctcaactctacgtctttgtttcgttttctgt |
| | tctgcgccgttacagatccaagctgtgaccggcgcctacGAATGCCGCCACCATG |
| | GCTgctgaaggTtccgtcgccaggcagcctgacctcttgacctgcgacgatgagc |
| | cgatccatatccccggtgccatccaaccgcatggactgctgctcgccctcgccgc |
| | cgacatgacgatcgttgccggcagcgacaaccttcccgaactcaccggactggcg |
| | atcggcgccctgatcggccgctctgcggccgatgtGttcgactcggaAacgcaca |
| | accgtctgacgatcgccttggccgagcccggggcggccgtcggagcaccgatcac |
| | tgtcggcttcacgatgcgaaaggacgcaggcttcatcggctcctggcatcgccat |
| | gatcagctcatcttcctGgagctGgagcctccccagcgggacgtcgccgagccgc |
| | aggcgttcttccgccgcaccaacagcgccatccgccgcctgcaggccgccgaaac |
| | cttggaaagcgcctgcgccgccgcggcgcaagaggtgcggaagattaccggcttc |
| | gatcgggtgatgatctatcgcttcgcctccgacttcagcggcgaagtgatcgcag |
| | aggatcggtgcgccgaggtcgagtcaaaactaggcctgcactatcctgcctcaac |
| | cgtgccggcgcaggcccgtcggctctataccatcaacccggtacggatcattccc |
| | gatatcaattatcggccggtgccggtcaccccagacctcaatccggtcaccgggc |
| | ggccgattgatcttagcttcgccatcctgcgcagcgtGtcgcccgtccatctgga |
| | Gttcatgcgcaacataggcatgcacggcacgatgtcgatctcgattttgcgcggc |
| | gagcgactgtggggattgatcgtttgccatcaccgaacgccgtactacgtcgatc |
| | tcgatggccgccaagcctgcgagctagtcgcccaggttctggcctggcagatcgg |
| | cgtgatggaagagTAAAACGBBABBABBABBABBABBACCATctgtgccttctag |
| | ttgccagccatctgttgtttgcccctcccccgtgccttccttgaccctggaaggt |
| | gccactcccactgtcctttcctaataaaatgaggaaattgcatcgcattgtctga |
| | gtaggtgtcattctattctggggggggggtggggcaggacagcaagggggaggat |
| | tgggaaggcaatagcaggcatgctggggatgcggtgggctctatggCAGTCAGTG |
| | CGGCCGCGCCTGGGCTTACttaattaacgagagcataatattgatatgtgccaaa |
| | gttgtttctgactgactaataagtataatttgtttctattatgtataggttaagc |
| | taattacttattttataatacaacatgactgtttttaaagtacaaaataagttta |
| | tttttgtaaaagagagaatgtttaaaagttttgttactttatagaagaaattttg |
| | agtttttgtttttttttaataaataaataaacataaataaattgtttgttgaatt |
| | tattattagtatgtaagtgtaaatataataaaacttaatatctattcaaattaat |
| | aaataaacctcgatatacagaccgataaaacacatgcgtcaattttacgcatgat |
| | tatctttaacgtacgtcacaatatgattatctttctagggttaaataatagtttc |
| | taatttttttattattcagcctgctgtcgtgaataccgagctccaattcgcccta |
| | tagtgagtcgtattacaattcactggccgtcgttttacaacgtcgtgactgggaa |
| | aaccctggcgttacccaacttaatcgccttgcagcacatccccctttcgccagct |
| | ggcgtaatagcgaagaggcccgcaccgatcgcccttcccaacagttgcgcagcct |
| | gaatggcgaatgggacgcgccctgtagcggcgcattaagcgcggcgggtgtggtg |
| | gttacgcgcagcgtgaccgctacacttgccagcgccctagcgcccgctcctttcg |
| | ctttcttcccttcctttctcgccacgttcgccggctttccccgtcaagctctaaa |
| | tcgggggctccctttagggttccgatttagtgctttacggcacctcgaccccaaa |
| | aaacttgattagggtgatggttcacgtagtgggccatcgccctgatagacggttt |
| | ttcgccctttgacgttggagtccacgttctttaatagtggactcttgttccaaac |
| | tggaacaacactcaaccctatctcggtctattcttttgatttataagggattttg |
| | ccgatttcggcctattggttaaaaaatgagctgatttaacaaaaatttaacgcga |
| | attttaacaaaatattaacgcttacaatttaggtggcacttttcggggaaatgtg |
| | cgcggaacccctatttgtttatttttctaaatacattcaaatatgtatccgctca |
| | tgagacaataaccctgataaatgcttcaataatattgaaaaaggaagagtatgag |
| | tattcaacatttccgtgtcgcccttattcccttttttgcggcattttgccttcct |
| | gtttttgctcacccagaaacgctggtgaaagtaaaagatgctgaagatcagttgg |
| | g |
| | (SEQ ID NO: 39) |
| pAJW_P116 | tgcacgagtgggttacatcgaactggatctcaacagcggtaagatccttgagagt |
| (CD47 + NQO1 + | tttcgccccgaagaacgttttccaatgatgagcacttttaaagttctgctatgtg |
| iCasp9) | gcgcggtattatcccgtattgacgccgggcaagagcaactcggtcgccgcataca |
| | ctattctcagaatgacttggttgagtactcaccagtcacagaaaagcatcttacg |
| | gatggcatgacagtaagagaattatgcagtgctgccataaccatgagtgataaca |
| | ctgcggccaacttacttctgacaacgatcggaggaccgaaggagctaaccgcttt |
| | tttgcacaacatgggggatcatgtaactcgccttgatcgttgggaaccggagctg |
| | aatgaagccataccaaacgacgagcgtgacaccacgatgcctgtagcaatggcaa |
| | caacgttgcgcaaactattaactggcgaactacttactctagcttcccggcaaca |
| | attaatagactggatggaggcggataaagttgcaggaccacttctgcgctcggcc |
| | cttccggctggctggtttattgctgataaatctggagccggtgagcgtgggtctc |
| | gcggtatcattgcagcactggggccagatggtaagccccccgtatcgtagttatc |
| | tacacgacggggagtcaggcaactatggatgaacgaaatagacagatcgctgaga |
| | taggtgcctcactgattaagcattggtaactgtcagaccaagtttactcatatat |
| | actttagattgatttaaaacttcatttttaatttaaaaggatctaggtgaagatc |
| | ctttttgataatctcatgaccaaaatcccttaacgtgagttttcgttccactgag |
| | cgtcagaccccgtagaaaagatcaaaggatcttcttgagatcctttttttctgcg |
| | cgtaatctgctgcttgcaaacaaaaaaaccaccgctaccagcggtggtttgtttg |
| | ccggatcaagagctaccaactctttttccgaaggtaactggcttcagcagagcgc |
| | agataccaaatactgttcttctagtgtagccgtagttaggccaccacttcaagaa |
| | ctctgtagcaccgcctacatacctcgctctgctaatcctgttaccagtggctgct |
| | gccagtggcgataagtcgtgtcttaccgggttggactcaagacgatagttaccgg |
| | ataaggcgcagcggtcgggctgaacggggggttcgtgcacacagcccagcttgga |
| | gcgaacgacctacaccgaactgagatacctacagcgtgagctatgagaaagcgcc |
| | acgcttcccgaagggagaaaggcggacaggtatccggtaagcggcagggtcggaa |
| | caggagagcgcacgagggagcttccagggggaaacgcctggtatctttatagtcc |
| | tgtcgggtttcgccacctctgacttgagcgtcgatttttgtgatgctcgtcaggg |
| | gggcggagcctatggaaaaacgccagcaacgcggcctttttacggttcctggcct |
| | tttgctggccttttgctcacatgttctttcctgcgttatcccctgattctgtgga |
| | taaccgtattaccgcctttgagtgagctgataccgctcgccgcagccgaacgacc |
| | gagcgcagcgagtcagtgagcgaggaagcggaagagcgcccaatacgcaaaccgc |
| | ctctccccgcgcgttggccgattcattaatgcagctggcacgacaggtttcccga |
| | ctggaaagcgggcagtgagcgcaacgcaattaatgtgagttagctcactcattag |
| | gcaccccaggctttacactttatgcttccggctcgtatgttgtgtggaattgtga |
| | gcggataacaatttcacacaggaaacagctatgaccatgattacgccaagctcga |
| | aattaaccctcactaaagggaacaaaagctggtacctcgcgcgacttggtttgcc |
| | attctttagcgcgcgtcgcgtcacacagcttggccacaatgtggtttttgtcaaa |
| | cgaagattctatgacgtgtttaaagtttaggtcgagtaaagcgcaaatctttttt |
| | aaccctagaaagatagtctgcgtaaaattgacgcatgcattcttgaaatattgct |
| | ctctctttctaaatagcgcgaatccgtcgctgtgcatttaggacatctcagtcgc |
| | cgcttggagctcccgtgaggcgtgcttgtcaatgcggtaagtgtcactgattttg |
| | aactataacgaccgcgtgagtcaaaatgacgcatgattatcttttacgtgacttt |
| | taagatttaactcatacgataattatattgttatttcatgttctacttacgtgat |
| | aacttattatatatatattttcttgttatagatatcatcaactttgtatagaaaa |
| | gttgctcgacattgattattgactagttattaatagtaatcaattacggggtcat |
| | tagttcatagcccatatatggagttccgcgttacataacttacggtaaatggccc |
| | gcctggctgaccgcccaacgacccccgcccattgacgtcaataatgacgtatgtt |
| | cccatagtaacgccaatagggactttccattgacgtcaatgggtggagtatttac |
| | ggtaaactgcccacttggcagtacatcaagtgtatcatatgccaagtacgccccc |
| | tattgacgtcaatgacggtaaatggcccgcctggcattatgcccagtacatgacc |
| | ttatgggactttcctacttggcagtacatctacgtattagtcatcgctattacca |
| | tggtcgaggtgagccccacgttctgcttcactctccccatctcccccccctcccc |
| | acccccaattttgtatttatttattttttaattattttgtgcagcgatgggggcg |
| | gggggggggggggggcgcgcgccaggcggggggggggggcgaggggggggcgggg |
| | cgaggcggagaggtgcggcggcagccaatcagagcggcgcgctccgaaagtttcc |
| | ttttatggcgaggcggcggcggcggcggccctataaaaagcgaagcgcgcggcgg |
| | gcgggagtcgctgcgcgctgccttcgccccgtgccccgctccgccgccgcctcgc |
| | gccgcccgccccggctctgactgaccgcgttactcccacaggtgagcgggcggga |
| | cggcccttctcctccgggctgtaattagcgcttggtttaatgacggcttgtttct |
| | tttctgtggctgcgtgaaagccttgaggggctccgggagggccctttgtgcgggg |
| | ggagcggctcggggggtgcgtgcgtgtgtgtgtgcgtggggagcgccgcgtgcgg |
| | ctccgcgctgcccggcggctgtgagcgctgcgggcgcggcgcggggctttgtgcg |
| | ctccgcagtgtgcgcgaggggagcgcggccgggggcggtgccccgcggtgcgggg |
| | ggggctgcgaggggaacaaaggctgcgtgcggggtgtgtgcgtgggggggtgagc |
| | agggggtgtgggcgcgtcggtcgggctgcaaccccccctgcacccccctccccga |
| | gttgctgagcacggcccggcttcgggtgcggggctccgtacggggcgtggcgcgg |
| | ggctcgccgtgccgggcggggggggcggcaggtgggggtgccgggcggggggggc |
| | cgcctcgggccggggagggctcgggggaggggcgcggcggcccccggagcgccgg |
| | cggctgtcgaggcgcggcgagccgcagccattgccttttatggtaatcgtgcgag |
| | agggcgcagggacttcctttgtcccaaatctgtgcggagccgaaatctgggaggc |
| | gccgccgcaccccctctagcgggcgcggggcgaagcggtgcggcgccggcaggaa |
| | ggaaatgggcggggagggccttcgtgcgtcgccgcgccgccgtccccttctccct |
| | ctccagcctcggggctgtccgcggggggacggctgccttcgggggggacggggca |
| | gggcggggttcggcttctggcgtgtgaccggcggctctagagcctctgctaacca |
| | tgttcatgccttcttctttttcctacagctcctgggcaacgtgctggttattgtg |
| | ctgtctcatcattttggcaaagaattgcaagtttgtacaaaaaagcaggctGAAT |
| | GCCGCCACCATGGCTaccgagtataaaccgacggtccgacttgcgactcgggatg |
| | acgttccacgcgccgtgaggactctggccgccgccttcgctgactacccggccac |
| | acggcacacagttgaccccgacagacacatagagcgggtcacagagttgcaagaa |
| | cttttcctgacgcgggttggActcgatattggaaaagtttgggttgccgacgatg |
| | gtgccgcagttgcggtgtggactactccagaaagtgtagaagctggagccgtatt |
| | cgctgaaatagggccacggatggcagagctgtctggtagcaggctcgcagcgcag |
| | cagcagatggaaggCcttctcgctccgcatcggcctaaggaaccggcgtggttcc |
| | tggctacggtgggagtctcacctgaccatcaaggaaagggattgggaagtgctgt |
| | cgttcttccaggcgtggaagcggccgaacgagcaggcgtcccagccttcttggaA |
| | acgagcgcacccaggaatcttccattctacgagcgactgggtttcactgtgacag |
| | cagacgtagaagtccctgaagggcctcggacttggtgcatgaccagaaagcctgg |
| | ggcttgaAACGCCATgatccagacatgataagatacattgatgagtttggacaaa |
| | ccacaactagaatgcagtgaGGCGCGCCTGGATACTTCTGCCTGCAGGGGAGTCG |
| | Ctcttttgcaatgctctttgggaaattatattagcctaattactaatttcctggc |
| | cctcaaggtgacttgcttgaacttgccacctTCGCCACTAGGGGGCAGCAttggt |
| | ttacacagggtgaacaaacgttcaattctaaactaatattctttggtgggaaagt |
| | gtgttcattttggtttgttttgaagaagtcacttcatacctttgaactggggtta |
| | agctggttaactcccaaaattccaccagttccaatatcctatggaaaaaccccaa |
| | aaccactCAAAggatctgcgatcgctccggtgcccgtcagtgggcagagcgcaca |
| | tcgcccacagtccccgagaagttggggggaggggtcggcaattgaacgggtgcct |
| | agagaaggtggcgcggggtaaactgggaaagtgatgtcgtgtactggctccgcct |
| | ttttcccgaggggggggagaaccgtatataagtgcagtagtcgccgtgaacgttc |
| | tttttcgcaacgggtttgccgccagaacacagctgaagcttcgaggggctcgcat |
| | ctctccttcacgcgcccgccgccctacctgaggccgccatccacgccggttgagt |
| | cgcgttctgccgcctcccgcctgtggtgcctcctgaactgcgtccgccgtctagg |
| | taagtttaaagctcaggtcgagTccgggcctttgtccggcgctcccttggagcct |
| | acctagactcagccggctctccacgctttgcctgaccctgcttgctcaactctac |
| | gtctttgtttcgttttctgttctgcgccgttacagatccaagctgtgaccggcgc |
| | ctacGAATGCCGCCACCATGTGGCCGTTGGTGGCCGCACTTCTGCTGGGTTCTGC |
| | ATGTTGTGGGAGCGCGCAGCTCCTTTTCAACAAAACTAAGAGTGTGGAATTTACA |
| | TTTTGCAACGATACAGTCGTAATCCCCTGCTTCGTGACTAACATGGAGGCCCAAA |
| | ATACCACAGAGGTTTACGTGAAATGGAAGTTTAAGGGCAGGGATATTTATACCTT |
| | CGATGGGGCTTTGAACAAGTCTACTGTGCCTACCGACTTCAGCTCCGCTAAGATT |
| | GAGGTGAGCCAGCTGCTCAAGGGCGACGCGAGTCTGAAAATGGACAAATCAGACG |
| | CCGTCAGTCATACGGGAAATTACACCTGCGAAGTCACTGAGCTGACCCGGGAAGG |
| | GGAGACTATAATTGAGTTGAAATATCGAGTTGTCTCCTGGTTTTCACCCAATGAA |
| | AACATCCTAATTGTTATATTCCCAATCTTCGCCATTCTGCTGTTCTGGGGCCAGT |
| | TTGGAATCAAGACGCTAAAATATCGCTCGGGAGGCATGGATGAAAAGACCATCGC |
| | ACTCCTTGTTGCCGGCTTGGTGATCACGGTTATCGTGATTGTGGGAGCGATCTTA |
| | TTTGTGCCTGGCGAGTATAGCCTCAAAAATGCTACAGGACTTGGTCTGATAGTAA |
| | CATCAACAGGGATTCTTATTCTCTTACACTACTACGTATTTTCTACCGCCATCGG |
| | CCTCACCTCCTTCGTGATCGCCATACTCGTGATTCAAGTGATCGCCTACATCCTG |
| | GCTGTCGTCGGCCTGTCTCTGTGCATCGCTGCCTGTATCCCCATGCACGGGCCAC |
| | TGCTGATTTCAGGACTCTCCATACTGGCTCTGGCCCAGTTGCTGGGTCTAGTCTA |
| | CATGAAGTTTGTAGCTTCCAATCAGAAAACTATTCAGCCACCCAGAAAGGCAGTG |
| | GAAGAGCCTTTAAATGCATTCAAAGAAAGCAAGGGGATGATGAACGACGAGTAAA |
| | ACGCCATgggggcatccctgtgacccctccccagtgcctctcctggccctggaag |
| | ttgccactccagtgcccaccagccttgtcctaataaaattaagttgcatcatttt |
| | gtctgactaggtgtccttctataatattatggggtggaggggggggtatggagca |
| | aggggcaagttgggaagagaacctgtagggcctgcggggtctattgggaaccaag |
| | ctggagtgcagtggcacaatcttggctcactgcaatctccgcctcctgggttcaa |
| | gcgattctcctgcctcagcctcccgagttgttgggattccaggcatgcatgacca |
| | ggctcagctaatttttgtttttttggtagagTcggggtttcaccatattggccag |
| | gctggtgtccaactcctaatctcaggtgatctacccaccttggcctcccaaattg |
| | ctgggattacaggcgtgaaccactgctcccttccctgtccttCAGTGGCATCGCt |
| | cttttgcaatgctctttgggaaattatattagcctaattactaatttcctggccc |
| | tcaaggtgacttgcttgaacttgccacctTCGCCACTAGGGGGCAGCAttggttt |
| | acacagggtgaacaaacgttcaattctaaactaatattctttggtgggaaagtgt |
| | gttcattttggtttgttttgaagaagtcacttcatacctttgaactggggttaag |
| | ctggttaactcccaaaattccaccagttccaatatcctatggaaaaaccccaaaa |
| | ccactCAAAggatctgcgatcgctccggtgcccgtcagtgggcagagcgcacatc |
| | gcccacagtccccgagaagttggggggaggggtcggcaattgaacgggtgcctag |
| | agaaggtggcgcggggtaaactgggaaagtgatgtcgtgtactggctccgccttt |
| | ttcccgagggtgggggagaaccgtatataagtgcagtagtcgccgtgaacgttct |
| | ttttcgcaacgggtttgccgccagaacacagctgaagcttcgaggggctcgcatc |
| | tctccttcacgcgcccgccgccctacctgaggccgccatccacgccggttgagtc |
| | gcgttctgccgcctcccgcctgtggtgcctcctgaactgcgtccgccgtctaggt |
| | aagtttaaagctcaggtcgagTccgggcctttgtccggcgctcccttggagccta |
| | cctagactcagccggctctccacgctttgcctgaccctgcttgctcaactctacg |
| | tctttgtttcgttttctgttctgcgccgttacagatccaagctgtgaccggcgcc |
| | tacGAATGCCGCCACCATGGTAGGCAGACGTGCGCTGATTGTGCTTGCGCATAGT |
| | GAGAGAACAAGTTTCAATTATGCAATGAAAGAGGCAGCTGCTGCAGCGCTCAAGA |
| | AAAAAGGGTGGGAGGTGGTAGAATCTGACCTGTACGCCATGAACTTCAACCCCAT |
| | TATTTCCCGCAAGGACATCACCGGGAAACTGAAAGATCCAGCAAACTTTCAATAC |
| | CCAGCTGAGTCTGTCCTAGCTTACAAGGAAGGCCATCTCAGTCCCGATATAGTGG |
| | CCGAACAGAAGAAACTCGAAGCTGCTGACTTAGTGATATTCCAATTCCCCTTGCA |
| | GTGGTTCGGCGTTCCCGCCATCCTGAAGGGTTGGTTTGAGAGGGTTTTTATTGGG |
| | GAGTTCGCATACACTTATGCTGCCATGTACGACAAGGGGCCATTTCGATCAAAGA |
| | AAGCCGTCTTGAGCATCACAACCGGAGGGTCCGGTAGCATGTACTCTTTACAGGG |
| | AATACACGGCGATATGAATGTGATCCTGTGGCCTATTCAGTCAGGCATCCTTCAC |
| | TTCTGCGGATTTCAGGTTCTGGAGCCTCAGCTTACATATAGCATCGGCCACACGC |
| | CTGCCGATGCCCGGATTCAGATTCTCGAGGGTTGGAAGAAAAGGCTGGAGAACAT |
| | CTGGGACGAAACCCCGCTGTATTTTGCCCCAAGCTCGCTGTTCGATTTGAACTTT |
| | CAGGCCGGTTTCCTAATGAAGAAGGAAGTGCAAGACGAAGAGAAAAATAAGAAGT |
| | TTGGACTCTCCGTCGGACATCACCTGGGCAAGTCCATCCCTACTGATAATCAGAT |
| | CAAGGCCCGGAAATAAAACGCCATgaattcactcctcaggtgcaggctgcctatc |
| | agaaggtggtggctggtgtggccaatgccctggctcacaaataccactgagatct |
| | ttttccctctgccaaaaattatggggacatcatgaagccccttgagcatctgact |
| | tctggctaataaaggaaatttattttcattgcaatagtgtgttggaattttttgt |
| | gtctctcactcggaaggacatatgggagggcaaatcatttaaaacatcagaatga |
| | gtatttggtttagagtttggcaacatatgcccatatgctggctgccatgaacaaa |
| | ggttggctataaagaggtcatcagtatatgaaacagccccctgctgtccattcct |
| | tattccatagaaaagccttgacttgaggttagattttttttatattttgttttgt |
| | gttatttttttctttaacatccctaaaattttccttacatgttttactagccaga |
| | tttttcctcctctcctgactactcccagtcatagctgtccctcttctcttatgga |
| | gatCAGTTCGCTCGCtcttttgcaatgctctttgggaaattatattagcctaatt |
| | actaatttcctggccctcaaggtgacttgcttgaacttgccacctTCGCCACTAG |
| | GGGGCAGCAttggtttacacagggtgaacaaacgttcaattctaaactaatattc |
| | tttggtgggaaagtgtgttcattttggtttgttttgaagaagtcacttcatacct |
| | ttgaactggggttaagctggttaactcccaaaattccaccagttccaatatccta |
| | tggaaaaaccccaaaaccactCAAAggatctgcgatcgctccggtgcccgtcagt |
| | gggcagagcgcacatcgcccacagtccccgagaagttggggggaggggtcggcaa |
| | ttgaacgggtgcctagagaaggtggcgcggggtaaactgggaaagtgatgtcgtg |
| | tactggctccgcctttttcccgaggggggggagaaccgtatataagtgcagtagt |
| | cgccgtgaacgttctttttcgcaacgggtttgccgccagaacacagctgaagctt |
| | cgaggggctcgcatctctccttcacgcgcccgccgccctacctgaggccgccatc |
| | cacgccggttgagtcgcgttctgccgcctcccgcctgtggtgcctcctgaactgc |
| | gtccgccgtctaggtaagtttaaagctcaggtcgagTccgggcctttgtccggcg |
| | ctcccttggagcctacctagactcagccggctctccacgctttgcctgaccctgc |
| | ttgctcaactctacgtctttgtttcgttttctgttctgcgccgttacagatccaa |
| | gctgtgaccggcgcctacGAATGCCGCCACCATGctGgagggagtgcaggtggag |
| | actatctccccaggtgatggcagaacgttccctaagagaggacaaacatgcgtgg |
| | tgcactacaccgggatgcttgaagatggaaagaaagttgattcctcccgggacag |
| | aaacaagccctttaagtttatgctaggcaagcaggaggtgatccgaggctgggaa |
| | gaaggggttgcccagatgagtgtgggtcagagagccaaactgactatatctccag |
| | attatgcctatggtgccactgggcacccaggcatcatcccaccacatgccactct |
| | cGTGTTCgatgtggagcttctaaaactggaatctggcggtggTtccggagtcgac |
| | ggatttggtgatgtcggtgctcttgagagtttgaggggaaatgcagatttggctt |
| | acatcctgagcatggagccctgtggccactgcctcattatcaacaatgtgaactt |
| | ctgccgtgagtccgggctccgcacccgcactggctccaacatcgactgtgagaag |
| | ttgcggcgtcgcttctcctcgctgcatttcatggtggaggtgaagggcgacctga |
| | ctgccaagaaaatggtgctggctttgctggagcttgcgaggcaagatcatggggc |
| | attggactgctgcgtggtagtcattctctctcacggctgtcaggccagccacctg |
| | cagttcccaggggctgtctacggcacagatggatgccctgtgtcggtcgagaaga |
| | ttgtgaacatcttcaatgggaccagctgccccagcctgggagggaagcccaagct |
| | ctttttcatccaggcctgtggtggggagcagaaagaccatgggtttgaggtggcc |
| | tccacttcccctGAGGACgagtcccctggcagtaaccccgagccagatgccaccc |
| | cgttccaggaaggtttgaggaccttcgaccagctggacgccatatctagtttgcc |
| | cacacccagtgacatctttgtgtcctactctactttcccaggttttgtttcctgg |
| | agggaccccaagagtggctcctggtacgttGAAACCctggacgacatctttgagc |
| | agtgggctcactctGAAGATctgcagtccctcctgcttagggtcgctaatgctgt |
| | ttcggtgaaagggatttataaacagatgcctggttgctttaatttcctccggaaa |
| | aaacttttctttaaaacatcagtcgactatcCGTATGacgtaccagactacgcac |
| | tcgacTAAAACGCCATgggtggcatccctgtgacccctccccagtgcctctcctg |
| | gccctggaagttgccactccagtgcccaccagccttgtcctaataaaattaagtt |
| | gcatcattttgtctgactaggtgtccttctataatattatggggtggaggggggt |
| | ggtatggagcaaggggcaagttgggaagagaacctgtagggcctgcggggtctat |
| | tgggaaccaagctggagtgcagtggcacaatcttggctcactgcaatctccgcct |
| | cctgggttcaagcgattctcctgcctcagcctcccgagttgttgggattccaggc |
| | atgcatgaccaggctcagctaatttttgtttttttggtagagTcggggtttcacc |
| | atattggccaggctggtgtccaactcctaatctcaggtgatctacccaccttggc |
| | ctcccaaattgctgggattacaggcgtgaaccactgctcccttccctgtccttct |
| | gattttaaaataactataccagcaggaggacgtccagacacagcataggctacct |
| | ggccatgcccaaccggtgggacatttgagttgcttgcttggcactgtcctctcat |
| | gcgttgggtccactcagtagatgcctgttCAGTCAGTTCGCtcttttgcaatgct |
| | ctttgggaaattatattagcctaattactaatttcctggccctcaaggtgacttg |
| | cttgaacttgccacctTCGCCACTAGGGGGCAGCAttggtttacacagggtgaac |
| | aaacgttcaattctaaactaatattctttggtgggaaagtgtgttcattttggtt |
| | tgttttgaagaagtcacttcatacctttgaactggggttaagctggttaactccc |
| | aaaattccaccagttccaatatcctatggaaaaaccccaaaaccactCAAAggat |
| | ctgcCatcgctccggtgcccgtcagtgggcagagcgcacatcgcccacagtcccc |
| | gagaagttggggggaggggtcggcaattgaacgggtgcctagagaaggtggcgcg |
| | gggtaaactgggaaagtgatgtcgtgtactggctccgcctttttcccgagggggg |
| | ggagaaccgtatataagtgcagtagtcgccgtgaacgttctttttcgcaacgggt |
| | ttgccgccagaacacagctgaagcttcgaggggctcgcatctctccttcacgcgc |
| | ccgccgccctacctgaggccgccatccacgccggttgagtcgcgttctgccgcct |
| | cccgcctgtggtgcctcctgaactgcgtccgccgtctaggtaagtttaaagctca |
| | ggtcgagTccgggcctttgtccggcgctcccttggagcctacctagactcagccg |
| | gctctccacgctttgcctgaccctgcttgctcaactctacgtctttgtttcgttt |
| | tctgttctgcgccgttacagatccaagctgtgaccggcgcctacGAATGCCGCCA |
| | CCATGGCTgctgaaggTtccgtcgccaggcagcctgacctcttgacctgcgacga |
| | tgagccgatccatatccccggtgccatccaaccgcatggactgctgctcgccctc |
| | gccgccgacatgacgatcgttgccggcagcgacaaccttcccgaactcaccggac |
| | tggcgatcggcgccctgatcggccgctctgcggccgatgtGttcgactcggaAac |
| | gcacaaccgtctgacgatcgccttggccgagcccggggcggccgtcggagcaccg |
| | atcactgtcggcttcacgatgcgaaaggacgcaggcttcatcggctcctggcatc |
| | gccatgatcagctcatcttcctGgagctGgagcctccccagcgggacgtcgccga |
| | gccgcaggcgttcttccgccgcaccaacagcgccatccgccgcctgcaggccgcc |
| | gaaaccttggaaagcgcctgcgccgccgcggcgcaagaggtgcggaagattaccg |
| | gcttcgatcgggtgatgatctatcgcttcgcctccgacttcagcggcgaagtgat |
| | cgcagaggatcggtgcgccgaggtcgagtcaaaactaggcctgcactatcctgcc |
| | tcaaccgtgccggcgcaggcccgtcggctctataccatcaacccggtacggatca |
| | ttcccgatatcaattatcggccggtgccggtcaccccagacctcaatccggtcac |
| | cgggcggccgattgatcttagcttcgccatcctgcgcagcgtGtcgcccgtccat |
| | ctggaGttcatgcgcaacataggcatgcacggcacgatgtcgatctcgattttgc |
| | gcggcgagcgactgtggggattgatcgtttgccatcaccgaacgccgtactacgt |
| | cgatctcgatggccgccaagcctgcgagctagtcgcccaggttctggcctggcag |
| | atcggcgtgatggaagagTAAAACGBBABBABBABBABBABBACCATctgtgcct |
| | tctagttgccagccatctgttgtttgcccctcccccgtgccttccttgaccctgg |
| | aaggtgccactcccactgtcctttcctaataaaatgaggaaattgcatcgcattg |
| | tctgagtaggtgtcattctattctggggggtggggggggcaggacagcaaggggg |
| | aggattgggaaggcaatagcaggcatgctggggatgcggtgggctctatggCAGT |
| | TAGGGCGGCCGCGCCTGGGCTTACttaattaacgagagcataatattgatatgtg |
| | ccaaagttgtttctgactgactaataagtataatttgtttctattatgtataggt |
| | taagctaattacttattttataatacaacatgactgtttttaaagtacaaaataa |
| | gtttatttttgtaaaagagagaatgtttaaaagttttgttactttatagaagaaa |
| | ttttgagtttttgtttttttttaataaataaataaacataaataaattgtttgtt |
| | gaatttattattagtatgtaagtgtaaatataataaaacttaatatctattcaaa |
| | ttaataaataaacctcgatatacagaccgataaaacacatgcgtcaattttacgc |
| | atgattatctttaacgtacgtcacaatatgattatctttctagggttaaataata |
| | gtttctaatttttttattattcagcctgctgtcgtgaataccgagctccaattcg |
| | ccctatagtgagtcgtattacaattcactggccgtcgttttacaacgtcgtgact |
| | gggaaaaccctggcgttacccaacttaatcgccttgcagcacatccccctttcgc |
| | cagctggcgtaatagcgaagaggcccgcaccgatcgcccttcccaacagttgcgc |
| | agcctgaatggcgaatgggacgcgccctgtagcggcgcattaagcgcggcgggtg |
| | tggtggttacgcgcagcgtgaccgctacacttgccagcgccctagcgcccgctcc |
| | tttcgctttcttcccttcctttctcgccacgttcgccggctttccccgtcaagct |
| | ctaaatcgggggctccctttagggttccgatttagtgctttacggcacctcgacc |
| | ccaaaaaacttgattagggtgatggttcacgtagtgggccatcgccctgatagac |
| | ggtttttcgccctttgacgttggagtccacgttctttaatagtggactcttgttc |
| | caaactggaacaacactcaaccctatctcggtctattcttttgatttataaggga |
| | ttttgccgatttcggcctattggttaaaaaatgagctgatttaacaaaaatttaa |
| | cgcgaattttaacaaaatattaacgcttacaatttaggtggcacttttcggggaa |
| | atgtgcgcggaacccctatttgtttatttttctaaatacattcaaatatgtatcc |
| | gctcatgagacaataaccctgataaatgcttcaataatattgaaaaaggaagagt |
| | atgagtattcaacatttccgtgtcgcccttattcccttttttgcggcattttgcc |
| | ttcctgtttttgctcacccagaaacgctggtgaaagtaaaagatgctgaagatca |
| | gttggg |
| | (SEQ ID NO: 40) |
| pAJW_P136 | TTTTATCTCTCATTCGAAAATTCCCAACATCGGGATTATATAACTGAAAAGTTT |
| (FUT7 + | TGGCGTAATGCGTTGGTCGCAGGAACCGTACCCGTAGTCCTCGGACCACCCCGCG |
| PSGL-1 + | CAACTTACGAGGCTTTTGTTCCCGCAGACGCATTCGTACACGTGGACGATTTCGG |
| CXCR4) | TTCCGCTCGGGAACTCGCTGCATTTCTGACAGGTATGAACGAATCTAGGTATCAG |
| | CGGTTCTTCGCGTGGCGGGATCGGCTTCGGGTCAGGTTGTTTACGGATTGGAGAG |
| | AGAGATTTTGCGCAATATGCGATAGGTATCCTCATCTGCCTAGATCCCAGGTGTA |
| | CGAAGATTTGGAAGGCTGGTTCCAAGCATAGAACGCCATgggtggcatccctgtg |
| | acccctccccagtgcctctcctggccctggaagttgccactccagtgcccaccag |
| | ccttgtcctaataaaattaagttgcatcattttgtctgactaggtgtccttctat |
| | aatattatggggtggaggggggtggtatggagcaaggggcaagttgggaagagaa |
| | cctgtagggcctgcggggtctattgggaaccaagctggagtgcagtggcacaatc |
| | ttggctcactgcaatctccgcctcctgggttcaagcgattctcctgcctcagcct |
| | cccgagttgttgggattccaggcatgcatgaccaggctcagctaatttttgtttt |
| | tttggtagagTcggggtttcaccatattggccaggctggtgtccaactcctaatc |
| | tcaggtgatctacccaccttggcctcccaaattgctgggattacaggcgtgaacc |
| | actgctcccttccctgtccttCAGTGGCATCGCtcttttgcaatgctctttggga |
| | aattatattagcctaattactaatttcctggccctcaaggtgacttgcttgaact |
| | tgccacctTCGCCACTAGGGGGCAGCAttggtttacacagggtgaacaaacgttc |
| | aattctaaactaatattctttggtgggaaagtgtgttcattttggtttgttttga |
| | agaagtcacttcatacctttgaactggggttaagctggttaactcccaaaattcc |
| | accagttccaatatcctatggaaaaaccccaaaaccactCAAAggatctgcgatc |
| | gctccggtgcccgtcagtgggcagagcgcacatcgcccacagtccccgagaagtt |
| | ggggggaggggtcggcaattgaacgggtgcctagagaaggtggcgcggggtaaac |
| | tgggaaagtgatgtcgtgtactggctccgcctttttcccgaggggggggagaacc |
| | gtatataagtgcagtagtcgccgtgaacgttctttttcgcaacgggtttgccgcc |
| | agaacacagctgaagcttcgaggggctcgcatctctccttcacgcgcccgccgcc |
| | ctacctgaggccgccatccacgccggttgagtcgcgttctgccgcctcccgcctg |
| | tggtgcctcctgaactgcgtccgccgtctaggtaagtttaaagctcaggtcgagT |
| | ccgggcctttgtccggcgctcccttggagcctacctagactcagccggctctcca |
| | cgctttgcctgaccctgcttgctcaactctacgtctttgtttcgttttctgttct |
| | gcgccgttacagatccaagctgtgaccggcgcctacGAATGCCGCCACCATGCCC |
| | CTTCAGCTGTTGTTGCTTTTGATACTGCTGGGACCTGGAAACTCACTTCAGTTGT |
| | GGGACACATGGGCCGATGAGGCGGAGAAGGCCCTGGGTCCCCTTCTCGCAAGAGA |
| | CAGACGGCAAGCGACTGAGTATGAATACCTCGATTATGATTTCCTCCCCGAGACt |
| | GAGCCTCCTGAGATGTTGCGCAACAGTACAGACACAACGCCATTGACTGGCCCCG |
| | GAACTCCAGAGAGCACCACGGTTGAGCCTGCTGCCCGGAGAAGTACTGGATTGGA |
| | TGCGGGCGGCGCTGTGACTGAATTGACCACTGAGCTGGCCAATATGGGGAATCTG |
| | TCCACAGACAGCGCGGCCATGGAGATACAAACGACACAGCCTGCGGCCACCGAGG |
| | CCCAGACTACACAGCCTGTACCGACAGAGGCCCAAACTACACCATTGGCAGCTAC |
| | GGAAGCTCAaACaACTCGAttgACcGCtACCGAgGCACAGACTACCCCTCTCGCT |
| | GCGACtGAAGCcCAaACGACaCCACCTGCaGCaACaGAAGCACAAACTACACAGC |
| | CCACAGGGTTGGAGGCGCAAACAACTGCGCCTGCTGCCATGGAAGCTCAAACAAC |
| | GGCACCGGCCGCAATGGAGGCaCAGACTACtCCtCCtGCaGCTATGGAGGCcCAA |
| | ACGACaCAGACGACTGCcATGGAGGCACAGACAACTGCACCAGAGGCCACGGAGG |
| | CACAAACAACACAACCAACTGCAACAGAGGCGCAGACGACGCCCCTCGCAGCAAT |
| | GGAAGCTCTTTCCACGGAACCCAGCGCAACCGAAGCACTCTCTATGGAGCCTACA |
| | ACCAAACGGGGCCTTTTTATTCCATTCTCTGTGTCATCCGTGACTCATAAGGGGA |
| | TACCTATGGCTGCGTCTAATCTCTCAGTCAATTATCCAGTTGGTGCGCCAGATCA |
| | CATTTCCGTGAAACAGTGTCTCCTTGCCATATTGATACTTGCCTTGGTCGCGACT |
| | ATTTTTTTCGTGTGTACCGTAGTGCTCGCGGTTCGCCTCTCTCGGAAGGGTCACA |
| | TGTACCCTGTTAGGAATTATTCTCCCACTGAAATGGTGTGCATCAGCAGTCTGCT |
| | GCCTGATGGTGGCGAGGGACCATCCGCTACAGCCAACGGAGGACTCTCTAAGGCT |
| | AAGTCTCCTGGACTGACCCCTGAGCCCCGCGAGGATAGGGAAGGtGACGATCTCA |
| | CCCTCCACTCCTTTCTGCCAtagAACGCCATgaattcactcctcaggtgcaggct |
| | gcctatcagaaggtggtggctggtgtggccaatgccctggctcacaaataccact |
| | gagatctttttccctctgccaaaaattatggggacatcatgaagccccttgagca |
| | tctgacttctggctaataaaggaaatttattttcattgcaatagtgtgttggaat |
| | tttttgtgtctctcactcggaaggacatatgggagggcaaatcatttaaaacatc |
| | agaatgagtatttggtttagagtttggcaacatatgcccatatgctggctgccat |
| | gaacaaaggttggctataaagaggtcatcagtatatgaaacagccccctgctgtc |
| | cattccttattccatagaaaagccttgacttgaggttagattttttttatatttt |
| | gttttgtgttatttttttctttaacatccctaaaattttccttacatgttttact |
| | agccagatttttcctcctctcctgactactcccagtcatagctgtccctcttctc |
| | ttatggagatCAGTTCGCTCGCtcttttgcaatgctctttgggaaattatattag |
| | cctaattactaatttcctggccctcaaggtgacttgcttgaacttgccacctTCG |
| | CCACTAGGGGGCAGCAttggtttacacagggtgaacaaacgttcaattctaaact |
| | aatattctttggtgggaaagtgtgttcattttggtttgttttgaagaagtcactt |
| | catacctttgaactggggttaagctggttaactcccaaaattccaccagttccaa |
| | tatcctatggaaaaaccccaaaaccactCAAAggatctgcgatcgctccggtgcc |
| | cgtcagtgggcagagcgcacatcgcccacagtccccgagaagttggggggagggg |
| | tcggcaattgaacgggtgcctagagaaggtggcgcggggtaaactgggaaagtga |
| | tgtcgtgtactggctccgcctttttcccgaggggggggagaaccgtatataagtg |
| | cagtagtcgccgtgaacgttctttttcgcaacgggtttgccgccagaacacagct |
| | gaagcttcgaggggctcgcatctctccttcacgcgcccgccgccctacctgaggc |
| | cgccatccacgccggttgagtcgcgttctgccgcctcccgcctgtggtgcctcct |
| | gaactgcgtccgccgtctaggtaagtttaaagctcaggtcgagTccgggcctttg |
| | tccggcgctcccttggagcctacctagactcagccggctctccacgctttgcctg |
| | accctgcttgctcaactctacgtctttgtttcgttttctgttctgcgccgttaca |
| | gatccaagctgtgaccggcgcctacGAATGCCGCCACCATGGAAGGAATTTCCAT |
| | CTATACCTCTGACAATTATACAGAAGAAATGGGAAGTGGAGATTACGATAGCATG |
| | AAAGAGCCATGCTTTCGCGAGGAGAACGCAAACTTTAACAAGATATTTCTGCCTA |
| | CGATTTATTCAATAATATTTCTGACCGGGATAGTCGGGAACGGTCTGGTTATACT |
| | TGTGATGGGATATCAAAAGAAGCTCCGCTCAATGACAGATAAATATCGGCTTCAT |
| | CTCAGCGTCGCTGATCTTCTTTTCGTGATTACCCTGCCTTTCTGGGCTGTGGACG |
| | CAGTTGCAAATTGGTATTTCGGTAATTTTCTGTGTAAAGCTGTGCACGTTATATA |
| | TACTGTTAATCTGTATTCCTCAGTGCTGATTCTCGCATTTATTAGCCTCGATAGG |
| | TATCTCGCGATTGTCCATGCTACCAATTCACAAAGACCCCGCAAACTTCTTGCCG |
| | AGAAAGTAGTGTACGTGGGAGTTTGGATACCCGCGCTGTTGCTCACCATACCTGA |
| | TTTTATTTTCGCTAATGTGTCTGAAGCTGACGATCGCTACATTTGCGATCGGTTT |
| | TATCCAAACGATCTGTGGGTCGTAGTTTTCCAATTCCAACATATTATGGTGGGAC |
| | TCATTCTTCCAGGGATCGTGATTCTCTCTTGTTACTGTATCATTATTAGCAAATT |
| | GAGTCATTCAAAAGGGCATCAAAAGAGAAAAGCGTTGAAAACAACCGTGATTCTG |
| | ATTCTTGCCTTCTTCGCGTGCTGGTTGCCGTATTATATCGGAATCTCTATTGATA |
| | GCTTTATTCTTCTCGAAATTATCAAACAGGGCTGCGAATTTGAAAATACGGTTCA |
| | TAAGTGGATCAGCATTACGGAAGCTCTCGCCTTCTTTCATTGCTGCCTCAATCCA |
| | ATTCTGTACGCCTTTCTGGGCGCTAAGTTCAAGACGTCCGCTCAACATGCCCTTA |
| | CTTCAGTCTCACGGGGTTCATCCCTTAAAATTCTGAGCAAGGGGAAAAGGGGTGG |
| | CCACAGCTCAGTGTCAACCGAATCAGAATCATCTTCTTTCCATAGCTCTTGAAAC |
| | GCCATgggtggcatccctgtgacccctccccagtgcctctcctggccctggaagt |
| | tgccactccagtgcccaccagccttgtcctaataaaattaagttgcatcattttg |
| | tctgactaggtgtccttctataatattatggggtggaggggggtggtatggagca |
| | aggggcaagttgggaagagaacctgtagggcctgcggggtctattgggaaccaag |
| | ctggagtgcagtggcacaatcttggctcactgcaatctccgcctcctgggttcaa |
| | gcgattctcctgcctcagcctcccgagttgttgggattccaggcatgcatgacca |
| | ggctcagctaatttttgtttttttggtagagTcggggtttcaccatattggccag |
| | gctggtgtccaactcctaatctcaggtgatctacccaccttggcctcccaaattg |
| | ctgggattacaggcgtgaaccactgctcccttccctgtccttctgattttaaaat |
| | aactataccagcaggaggacgtccagacacagcataggctacctggccatgccca |
| | accggtgggacatttgagttgcttgcttggcactgtcctctcatgcgttgggtcc |
| | actcagtagatgcctgttCAGTCAGTTCGCtcttttgcaatgctctttgggaaat |
| | tatattagcctaattactaatttcctggccctcaaggtgacttgcttgaacttgc |
| | cacctTCGCCACTAGGGGGCAGCAttggtttacacagggtgaacaaacgttcaat |
| | tctaaactaatattctttggtgggaaagtgtgttcattttggtttgttttgaaga |
| | agtcacttcatacctttgaactggggttaagctggttaactcccaaaattccacc |
| | agttccaatatcctatggaaaaaccccaaaaccactCAAAggatctgcCatcgct |
| | ccggtgcccgtcagtgggcagagcgcacatcgcccacagtccccgagaagttggg |
| | gggaggggtcggcaattgaacgggtgcctagagaaggtggcgcggggtaaactgg |
| | gaaagtgatgtcgtgtactggctccgcctttttcccgaggggggggagaaccgta |
| | tataagtgcagtagtcgccgtgaacgttctttttcgcaacgggtttgccgccaga |
| | acacagctgaagcttcgaggggctcgcatctctccttcacgcgcccgccgcccta |
| | cctgaggccgccatccacgccggttgagtcgcgttctgccgcctcccgcctgtgg |
| | tgcctcctgaactgcgtccgccgtctaggtaagtttaaagctcaggtcgagTccg |
| | ggcctttgtccggcgctcccttggagcctacctagactcagccggctctccacgc |
| | tttgcctgaccctgcttgctcaactctacgtctttgtttcgttttctgttctgcg |
| | ccgttacagatccaagctgtgaccggcgcctacGAATGCCGCCACCATGGCTgct |
| | gaaggTtccgtcgccaggcagcctgacctcttgacctgcgacgatgagccgatcc |
| | atatccccggtgccatccaaccgcatggactgctgctcgccctcgccgccgacat |
| | gacgatcgttgccggcagcgacaaccttcccgaactcaccggactggcgatcggc |
| | gccctgatcggccgctctgcggccgatgtGttcgactcggaAacgcacaaccgtc |
| | tgacgatcgccttggccgagcccggggcggccgtcggagcaccgatcactgtcgg |
| | cttcacgatgcgaaaggacgcaggcttcatcggctcctggcatcgccatgatcag |
| | ctcatcttcctGgagctGgagcctccccagcgggacgtcgccgagccgcaggcgt |
| | tcttccgccgcaccaacagcgccatccgccgcctgcaggccgccgaaaccttgga |
| | aagcgcctgcgccgccgcggcgcaagaggtgcggaagattaccggcttcgatcgg |
| | gtgatgatctatcgcttcgcctccgacttcagcggcgaagtgatcgcagaggatc |
| | ggtgcgccgaggtcgagtcaaaactaggcctgcactatcctgcctcaaccgtgcc |
| | ggcgcaggcccgtcggctctataccatcaacccggtacggatcattcccgatatc |
| | aattatcggccggtgccggtcaccccagacctcaatccggtcaccgggcggccga |
| | ttgatcttagcttcgccatcctgcgcagcgtGtcgcccgtccatctggaGttcat |
| | gcgcaacataggcatgcacggcacgatgtcgatctcgattttgcgcggcgagcga |
| | ctgtggggattgatcgtttgccatcaccgaacgccgtactacgtcgatctcgatg |
| | gccgccaagcctgcgagctagtcgcccaggttctggcctggcagatcggcgtgat |
| | ggaagagTAAAACGBBABBABBABBABBABBACCATctgtgccttctagttgcca |
| | gccatctgttgtttgcccctcccccgtgccttccttgaccctggaaggtgccact |
| | cccactgtcctttcctaataaaatgaggaaattgcatcgcattgtctgagtaggt |
| | gtcattctattctggggggtggggtggggcaggacagcaagggggaggattggga |
| | aggcaatagcaggcatgctggggatgcggtgggctctatggCAGTTAGGGCGGCC |
| | GCGCCTGGGCTTACttaattaacgagagcataatattgatatgtgccaaagttgt |
| | ttctgactgactaataagtataatttgtttctattatgtataggttaagctaatt |
| | acttattttataatacaacatgactgtttttaaagtacaaaataagtttattttt |
| | gtaaaagagagaatgtttaaaagttttgttactttatagaagaaattttgagttt |
| | ttgtttttttttaataaataaataaacataaataaattgtttgttgaatttatta |
| | ttagtatgtaagtgtaaatataataaaacttaatatctattcaaattaataaata |
| | aacctcgatatacagaccgataaaacacatgcgtcaattttacgcatgattatct |
| | ttaacgtacgtcacaatatgattatctttctagggttaaataatagtttctaatt |
| | tttttattattcagcctgctgtcgtgaataccgagctccaattcgccctatagtg |
| | agtcgtattacaattcactggccgtcgttttacaacgtcgtgactgggaaaaccc |
| | tggcgttacccaacttaatcgccttgcagcacatccccctttcgccagctggcgt |
| | aatagcgaagaggcccgcaccgatcgcccttcccaacagttgcgcagcctgaatg |
| | gcgaatgggacgcgccctgtagcggcgcattaagcgcggcgggtgtggtggttac |
| | gcgcagcgtgaccgctacacttgccagcgccctagcgcccgctcctttcgctttc |
| | ttcccttcctttctcgccacgttcgccggctttccccgtcaagctctaaatcggg |
| | ggctccctttagggttccgatttagtgctttacggcacctcgaccccaaaaaact |
| | tgattagggtgatggttcacgtagtgggccatcgccctgatagacggtttttcgc |
| | cctttgacgttggagtccacgttctttaatagtggactcttgttccaaactggaa |
| | caacactcaaccctatctcggtctattcttttgatttataagggattttgccgat |
| | ttcggcctattggttaaaaaatgagctgatttaacaaaaatttaacgcgaatttt |
| | aacaaaatattaacgcttacaatttaggtggcacttttcggggaaatgtgcgcgg |
| | aacccctatttgtttatttttctaaatacattcaaatatgtatccgctcatgaga |
| | caataaccctgataaatgcttcaataatattgaaaaaggaagagtatgagtattc |
| | aacatttccgtgtcgcccttattcccttttttgcggcattttgccttcctgtttt |
| | tgctcacccagaaacgctggtgaaagtaaaagatgctgaagatcagttgggtgca |
| | cgagtgggttacatcgaactggatctcaacagcggtaagatccttgagagttttc |
| | gccccgaagaacgttttccaatgatgagcacttttaaagttctgctatgtggcgc |
| | ggtattatcccgtattgacgccgggcaagagcaactcggtcgccgcatacactat |
| | tctcagaatgacttggttgagtactcaccagtcacagaaaagcatcttacggatg |
| | gcatgacagtaagagaattatgcagtgctgccataaccatgagtgataacactgc |
| | ggccaacttacttctgacaacgatcggaggaccgaaggagctaaccgcttttttg |
| | cacaacatgggggatcatgtaactcgccttgatcgttgggaaccggagctgaatg |
| | aagccataccaaacgacgagcgtgacaccacgatgcctgtagcaatggcaacaac |
| | gttgcgcaaactattaactggcgaactacttactctagcttcccggcaacaatta |
| | atagactggatggaggcggataaagttgcaggaccacttctgcgctcggcccttc |
| | cggctggctggtttattgctgataaatctggagccggtgagcgtgggtctcgcgg |
| | tatcattgcagcactggggccagatggtaagccctcccgtatcgtagttatctac |
| | acgacggggagtcaggcaactatggatgaacgaaatagacagatcgctgagatag |
| | gtgcctcactgattaagcattggtaactgtcagaccaagtttactcatatatact |
| | ttagattgatttaaaacttcatttttaatttaaaaggatctaggtgaagatcctt |
| | tttgataatctcatgaccaaaatcccttaacgtgagttttcgttccactgagcgt |
| | cagaccccgtagaaaagatcaaaggatcttcttgagatcctttttttctgcgcgt |
| | aatctgctgcttgcaaacaaaaaaaccaccgctaccagcggtggtttgtttgccg |
| | gatcaagagctaccaactctttttccgaaggtaactggcttcagcagagcgcaga |
| | taccaaatactgttcttctagtgtagccgtagttaggccaccacttcaagaactc |
| | tgtagcaccgcctacatacctcgctctgctaatcctgttaccagtggctgctgcc |
| | agtggcgataagtcgtgtcttaccgggttggactcaagacgatagttaccggata |
| | aggcgcagcggtcgggctgaacggggggttcgtgcacacagcccagcttggagcg |
| | aacgacctacaccgaactgagatacctacagcgtgagctatgagaaagcgccacg |
| | cttcccgaagggagaaaggcggacaggtatccggtaagcggcagggtcggaacag |
| | gagagcgcacgagggagcttccagggggaaacgcctggtatctttatagtcctgt |
| | cgggtttcgccacctctgacttgagcgtcgatttttgtgatgctcgtcagggggg |
| | cggagcctatggaaaaacgccagcaacgcggcctttttacggttcctggcctttt |
| | gctggccttttgctcacatgttctttcctgcgttatcccctgattctgtggataa |
| | ccgtattaccgcctttgagtgagctgataccgctcgccgcagccgaacgaccgag |
| | cgcagcgagtcagtgagcgaggaagcggaagagcgcccaatacgcaaaccgcctc |
| | tccccgcgcgttggccgattcattaatgcagctggcacgacaggtttcccgactg |
| | gaaagcgggcagtgagcgcaacgcaattaatgtgagttagctcactcattaggca |
| | ccccaggctttacactttatgcttccggctcgtatgttgtgtggaattgtgagcg |
| | gataacaatttcacacaggaaacagctatgaccatgattacgccaagctcgaaat |
| | taaccctcactaaagggaacaaaagctggtacctcgcgcgacttggtttgccatt |
| | ctttagcgcgcgtcgcgtcacacagcttggccacaatgtggtttttgtcaaacga |
| | agattctatgacgtgtttaaagtttaggtcgagtaaagcgcaaatcttttttaac |
| | cctagaaagatagtctgcgtaaaattgacgcatgcattcttgaaatattgctctc |
| | tctttctaaatagcgcgaatccgtcgctgtgcatttaggacatctcagtcgccgc |
| | ttggagctcccgtgaggcgtgcttgtcaatgcggtaagtgtcactgattttgaac |
| | tataacgaccgcgtgagtcaaaatgacgcatgattatcttttacgtgacttttaa |
| | gatttaactcatacgataattatattgttatttcatgttctacttacgtgataac |
| | ttattatatatatattttcttgttatagatatcatcaactttgtatagaaaagtt |
| | gctcgacattgattattgactagttattaatagtaatcaattacggggtcattag |
| | ttcatagcccatatatggagttccgcgttacataacttacggtaaatggcccgcc |
| | tggctgaccgcccaacgacccccgcccattgacgtcaataatgacgtatgttccc |
| | atagtaacgccaatagggactttccattgacgtcaatgggtggagtatttacggt |
| | aaactgcccacttggcagtacatcaagtgtatcatatgccaagtacgccccctat |
| | tgacgtcaatgacggtaaatggcccgcctggcattatgcccagtacatgacctta |
| | tgggactttcctacttggcagtacatctacgtattagtcatcgctattaccatgg |
| | tcgaggtgagccccacgttctgcttcactctccccatctcccccccctccccacc |
| | cccaattttgtatttatttattttttaattattttgtgcagcgatgggggcgggg |
| | ggggggggggggcgcgcgccaggcggggcggggcggggcgaggggggggcggggc |
| | gaggcggagaggtgcggcggcagccaatcagagcggcgcgctccgaaagtttcct |
| | tttatggcgaggcggcggcggcggcggccctataaaaagcgaagcgcgcgggggg |
| | ggagtcgctgcgcgctgccttcgccccgtgccccgctccgccgccgcctcgcgcc |
| | gcccgccccggctctgactgaccgcgttactcccacaggtgagcgggcgggacgg |
| | cccttctcctccgggctgtaattagcgcttggtttaatgacggcttgtttctttt |
| | ctgtggctgcgtgaaagccttgaggggctccgggagggccctttgtgcgggggga |
| | gcggctcggggggtgcgtgcgtgtgtgtgtgcgtggggagcgccgcgtgcggctc |
| | cgcgctgcccggcggctgtgagcgctgcgggcgcggcgcggggctttgtgcgctc |
| | cgcagtgtgcgcgaggggagcgcggccgggggcggtgccccgcggtgcggggggg |
| | gctgcgaggggaacaaaggctgcgtgcggggtgtgtgcgtgggggggtgagcagg |
| | gggtgtgggcgcgtcggtcgggctgcaaccccccctgcacccccctccccgagtt |
| | gctgagcacggcccggcttcgggtgcggggctccgtacggggcgtggcgcggggc |
| | tcgccgtgccgggcggggggggcggcaggtgggggtgccgggcggggcggggccg |
| | cctcgggccggggagggctcgggggaggggcgcggcggcccccggagcgccggcg |
| | gctgtcgaggcgcggcgagccgcagccattgccttttatggtaatcgtgcgagag |
| | ggcgcagggacttcctttgtcccaaatctgtgcggagccgaaatctgggaggcgc |
| | cgccgcaccccctctagcgggcgcggggcgaagcggtgcggcgccggcaggaagg |
| | aaatgggcggggagggccttcgtgcgtcgccgcgccgccgtccccttctccctct |
| | ccagcctcggggctgtccgcggggggacggctgccttcgggggggacggggcagg |
| | gcggggttcggcttctggcgtgtgaccggcggctctagagcctctgctaaccatg |
| | ttcatgccttcttctttttcctacagctcctgggcaacgtgctggttattgtgct |
| | gtctcatcattttggcaaagaattgcaagtttgtacaaaaaagcaggctGAATGC |
| | CGCCACCATGGCTaccgagtataaaccgacggtccgacttgcgactcgggatgac |
| | gttccacgcgccgtgaggactctggccgccgccttcgctgactacccggccacac |
| | ggcacacagttgaccccgacagacacatagagcgggtcacagagttgcaagaact |
| | tttcctgacgcgggttggActcgatattggaaaagtttgggttgccgacgatggt |
| | gccgcagttgcggtgtggactactccagaaagtgtagaagctggagccgtattcg |
| | ctgaaatagggccacggatggcagagctgtctggtagcaggctcgcagcgcagca |
| | gcagatggaaggCcttctcgctccgcatcggcctaaggaaccggcgtggttcctg |
| | gctacggtgggagtctcacctgaccatcaaggaaagggattgggaagtgctgtcg |
| | ttcttccaggcgtggaagcggccgaacgagcaggcgtcccagccttcttggaAac |
| | gagcgcacccaggaatcttccattctacgagcgactgggtttcactgtgacagca |
| | gacgtagaagtccctgaagggcctcggacttggtgcatgaccagaaagcctgggg |
| | cttgaAACGCCATgatccagacatgataagatacattgatgagtttggacaaacc |
| | acaactagaatgcagtgaGGCGCGCCTGGATACTTCTGCCTGCAGGGGAGTCGCt |
| | cttttgcaatgctctttgggaaattatattagcctaattactaatttcctggccc |
| | tcaaggtgacttgcttgaacttgccacctTCGCCACTAGGGGGCAGCAttggttt |
| | acacagggtgaacaaacgttcaattctaaactaatattctttggtgggaaagtgt |
| | gttcattttggtttgttttgaagaagtcacttcatacctttgaactggggttaag |
| | ctggttaactcccaaaattccaccagttccaatatcctatggaaaaaccccaaaa |
| | ccactCAAAggggttggggttgcgccttttccaaggcagccctgggtttgcgcag |
| | ggacgcggctgctctgggcgtggttccgggaaacgcagcggcgccgaccctgggT |
| | ctGgcacattcttcacgtccgttcgcagcgtcacccggatcttcgccgctaccct |
| | tgtgggccccccggcgacgcttcctgctccgcccctaagtcgggaaggttccttg |
| | cggttcgcggcgtgccggacgtgacaaacggaagccgcacgTctGactagtaccc |
| | tcgcagacggacagcgccagggagcaatggcagcgcgccgaccgcgatgggctgt |
| | ggccaatagcggctgctcagcAgggcgcgccgagagcagcggccgggaaggggcg |
| | gtgcgggaggcggggtgtggggcggtagtgtgggccctgttcctgcccgcgcggt |
| | gttccgcattctgcaagcctccggagcgcacgtcggcagtcggctccctcgttga |
| | ccgaatcaccgacctctctccccagGAATGCCGCCACCATGAATAACGCCGGCCA |
| | CGGACCAACTAGACGCTTGAGGGGACTCGGAGTTTTGGCTGGAGTCGCACTCCTG |
| | GCCGCTCTGTGGCTGCTTTGGCTCCTTGGAAGCGCGCCAAGGGGCACACCTGCAC |
| | CACAACCTACCATAACAATTCTGGTGTGGCATTGGCCATTTACCGATCAACCACC |
| | CGAATTGCCGTCAGATACATGTACCAGGTATGGGATTGCACGGTGTCATCTCAGC |
| | GCGAATCGCTCATTGTTGGCTTCAGCAGATGCGGTAGTGTTTCATCATAGAGAAC |
| | TTCAAACTCGCAGGAGCCACCTTCCTTTGGCACAAAGGCCGCGGGGTCAACCGTG |
| | GGTTTGGGCATCAATGGAAAGTCCCTCACATACTCATGGGCTGTCACATCTGCGG |
| | GGAATCTTTAATTGGGTACTTTCCTATCGACGGGATAGCGATATTTTCGTCCCAT |
| | ACGGACGATTGGAACCTCATTGGGGTCCTTCTCCCCCTCTCCCTGCAAAATCCAG |
| | AGTTGCAGCTTGGGTCGTTTCCAATTTTCAAGAACGCCAACTTAGGGCTCGCCTC |
| | TATAGACAACTCGCTCCCCACCTTCGTGTTGACGTTTTCGGTCGAGCTAACGGGC |
| | GCCCGTTGTGTGCGTCATGTCTCGTACCTACGGTTGCTCAATATCGG |
| | (SEQ ID NO: 41) |
| pAJW_P156 | AGCAGATGCGGTAGTGTTTCATCATAGAGAACTTCAAACTCGCAGGAGCCACCTT |
| (FUT7 + PSGL-1 + | CCTTTGGCACAAAGGCCGCGGGGTCAACCGTGGGTTTGGGCATCAATGGAAAGTC |
| IntaL + | CCTCACATACTCATGGGCTGTCACATCTGCGGGGAATCTTTAATTGGGTACTTTC |
| IntB2 | CTATCGACGGGATAGCGATATTTTCGTCCCATACGGACGATTGGAACCTCATTGG |
| CCR2) | GGTCCTTCTCCCCCTCTCCCTGCAAAATCCAGAGTTGCAGCTTGGGTCGTTTCCA |
| | ATTTTCAAGAACGCCAACTTAGGGCTCGCCTCTATAGACAACTCGCTCCCCACCT |
| | TCGTGTTGACGTTTTCGGTCGAGCTAACGGGCGCCCGTTGTGTGCGTCATGTCTC |
| | GTACCTACGGTTGCTCAATATCGGTTTTATCTCTCATTCGAAAATTCCCAACATC |
| | GGGATTATATAACTGAAAAGTTTTGGCGTAATGCGTTGGTCGCAGGAACCGTACC |
| | CGTAGTCCTCGGACCACCCCGCGCAACTTACGAGGCTTTTGTTCCCGCAGACGCA |
| | TTCGTACACGTGGACGATTTCGGTTCCGCTCGGGAACTCGCTGCATTTCTGACAG |
| | GTATGAACGAATCTAGGTATCAGCGGTTCTTCGCGTGGCGGGATCGGCTTCGGGT |
| | CAGGTTGTTTACGGATTGGAGAGAGAGATTTTGCGCAATATGCGATAGGTATCCT |
| | CATCTGCCTAGATCCCAGGTGTACGAAGATTTGGAAGGCTGGTTCCAAGCATAGA |
| | ACGCCATgggtggcatccctgtgacccctccccagtgcctctcctggccctggaa |
| | gttgccactccagtgcccaccagccttgtcctaataaaattaagttgcatcattt |
| | tgtctgactaggtgtccttctataatattatggggtggaggggggtggtatggag |
| | caaggggcaagttgggaagagaacctgtagggcctgcggggtctattgggaacca |
| | agctggagtgcagtggcacaatcttggctcactgcaatctccgcctcctgggttc |
| | aagcgattctcctgcctcagcctcccgagttgttgggattccaggcatgcatgac |
| | caggctcagctaatttttgtttttttggtagagTcggggtttcaccatattggcc |
| | aggctggtgtccaactcctaatctcaggtgatctacccaccttggcctcccaaat |
| | tgctgggattacaggcgtgaaccactgctcccttccctgtccttCAGTGGCATCG |
| | Ctcttttgcaatgctctttgggaaattatattagcctaattactaatttcctggc |
| | cctcaaggtgacttgcttgaacttgccacctTCGCCACTAGGGGGCAGCAttggt |
| | ttacacagggtgaacaaacgttcaattctaaactaatattctttggtgggaaagt |
| | gtgttcattttggtttgttttgaagaagtcacttcatacctttgaactggggtta |
| | agctggttaactcccaaaattccaccagttccaatatcctatggaaaaaccccaa |
| | aaccactCAAAggatctgcgatcgctccggtgcccgtcagtgggcagagcgcaca |
| | tcgcccacagtccccgagaagttggggggaggggtcggcaattgaacgggtgcct |
| | agagaaggtggcgcggggtaaactgggaaagtgatgtcgtgtactggctccgcct |
| | ttttcccgaggggggggagaaccgtatataagtgcagtagtcgccgtgaacgttc |
| | tttttcgcaacgggtttgccgccagaacacagctgaagcttcgaggggctcgcat |
| | ctctccttcacgcgcccgccgccctacctgaggccgccatccacgccggttgagt |
| | cgcgttctgccgcctcccgcctgtggtgcctcctgaactgcgtccgccgtctagg |
| | taagtttaaagctcaggtcgagTccgggcctttgtccggcgctcccttggagcct |
| | acctagactcagccggctctccacgctttgcctgaccctgcttgctcaactctac |
| | gtctttgtttcgttttctgttctgcgccgttacagatccaagctgtgaccggcgc |
| | ctacGAATGCCGCCACCATGCCCCTTCAGCTGTTGTTGCTTTTGATACTGCTGGG |
| | ACCTGGAAACTCACTTCAGTIGTGGGACACATGGGCCGATGAGGCGGAGAAGGCC |
| | CTGGGTCCCCTTCTCGCAAGAGACAGACGGCAAGCGACTGAGTATGAATACCTCG |
| | ATTATGATTTCCTCCCCGAGACtGAGCCTCCTGAGATGTTGCGCAACAGTACAGA |
| | CACAACGCCATTGACTGGCCCCGGAACTCCAGAGAGCACCACGGTTGAGCCTGCT |
| | GCCCGGAGAAGTACTGGATTGGATGCGGGCGGCGCTGTGACTGAATTGACCACTG |
| | AGCTGGCCAATATGGGGAATCTGTCCACAGACAGCGCGGCCATGGAGATACAAAC |
| | GACACAGCCTGCGGCCACCGAGGCCCAGACTACACAGCCTGTACCGACAGAGGCC |
| | CAAACTACACCATTGGCAGCTACGGAAGCTCAaACaACTCGAttgACcGCtACCG |
| | AgGCACAGACTACCCCTCTCGCTGCGACtGAAGCcCAaACGACaCCACCTGCaGC |
| | aACaGAAGCACAAACTACACAGCCCACAGGGTTGGAGGCGCAAACAACTGCGCCT |
| | GCTGCCATGGAAGCTCAAACAACGGCACCGGCCGCAATGGAGGCaCAGACTACtC |
| | CtCCtGCaGCTATGGAGGCcCAAACGACaCAGACGACTGCcATGGAGGCACAGAC |
| | AACTGCACCAGAGGCCACGGAGGCACAAACAACACAACCAACTGCAACAGAGGCG |
| | CAGACGACGCCCCTCGCAGCAATGGAAGCTCTTTCCACGGAACCCAGCGCAACCG |
| | AAGCACTCTCTATGGAGCCTACAACCAAACGGGGCCTTTTTATTCCATTCTCTGT |
| | GTCATCCGTGACTCATAAGGGGATACCTATGGCTGCGTCTAATCTCTCAGTCAAT |
| | TATCCAGTTGGTGCGCCAGATCACATTTCCGTGAAACAGTGTCTCCTTGCCATAT |
| | TGATACTTGCCTTGGTCGCGACTATTTTTTTCGTGTGTACCGTAGTGCTCGCGGT |
| | TCGCCTCTCTCGGAAGGGTCACATGTACCCTGTTAGGAATTATTCTCCCACTGAA |
| | ATGGTGTGCATCAGCAGTCTGCTGCCTGATGGTGGCGAGGGACCATCCGCTACAG |
| | CCAACGGAGGACTCTCTAAGGCTAAGTCTCCTGGACTGACCCCTGAGCCCCGCGA |
| | GGATAGGGAAGGtGACGATCTCACCCTCCACTCCTTTCTGCCAtagAACGCCATg |
| | aattcactcctcaggtgcaggctgcctatcagaaggtggtggctggtgtggccaa |
| | tgccctggctcacaaataccactgagatctttttccctctgccaaaaattatggg |
| | gacatcatgaagccccttgagcatctgacttctggctaataaaggaaatttattt |
| | tcattgcaatagtgtgttggaattttttgtgtctctcactcggaaggacatatgg |
| | gagggcaaatcatttaaaacatcagaatgagtatttggtttagagtttggcaaca |
| | tatgcccatatgctggctgccatgaacaaaggttggctataaagaggtcatcagt |
| | atatgaaacagccccctgctgtccattccttattccatagaaaagccttgacttg |
| | aggttagattttttttatattttgttttgtgttatttttttctttaacatcccta |
| | aaattttccttacatgttttactagccagatttttcctcctctcctgactactcc |
| | cagtcatagctgtccctcttctcttatggagatCAGTTCGCTCGCtcttttgcaa |
| | tgctctttgggaaattatattagcctaattactaatttcctggccctcaaggtga |
| | cttgcttgaacttgccacctTCGCCACTAGGGGGCAGCAttggtttacacagggt |
| | gaacaaacgttcaattctaaactaatattctttggtgggaaagtgtgttcatttt |
| | ggtttgttttgaagaagtcacttcatacctttgaactggggttaagctggttaac |
| | tcccaaaattccaccagttccaatatcctatggaaaaaccccaaaaccactCAAA |
| | ggatctgcgatcgctccggtgcccgtcagtgggcagagcgcacatcgcccacagt |
| | ccccgagaagttggggggaggggtcggcaattgaacgggtgcctagagaaggtgg |
| | cgcggggtaaactgggaaagtgatgtcgtgtactggctccgcctttttcccgagg |
| | gtgggggagaaccgtatataagtgcagtagtcgccgtgaacgttctttttcgcaa |
| | cgggtttgccgccagaacacagctgaagcttcgaggggctcgcatctctccttca |
| | cgcgcccgccgccctacctgaggccgccatccacgccggttgagtcgcgttctgc |
| | cgcctcccgcctgtggtgcctcctgaactgcgtccgccgtctaggtaagtttaaa |
| | gctcaggtcgagTccgggcctttgtccggcgctcccttggagcctacctagactc |
| | agccggctctccacgctttgcctgaccctgcttgctcaactctacgtctttgttt |
| | cgttttctgttctgcgccgttacagatccaagctgtgaccggcgcctacGAATGC |
| | CGCCACCATGCTGTCAACATCCCGGTCTAGATTCATAAGAAACACCAATGAGAGT |
| | GGCGAAGAGGTGACCACTTTCTTCGATTATGATTATGGAGCCCCCTGTCATAAAT |
| | TTGACGTCAAACAGATCGGTGCACAGCTGCTGCCACCTCTTTATAGCCTCGTGTT |
| | TATTTTCGGGTTCGTGGGGAATATGCTCGTGGTTTTGATCCTCATCAACTGCAAG |
| | AAGCTCAAATGTCTGACCGATATATACCTACTGAACCTGGCCATTTCAGATCTGC |
| | TCTTCCTGATTACGCTGCCACTCTGGGCACATTCGGCCGCTAACGAATGGGTGTT |
| | TGGTAACGCTATGTGCAAGCTGTTCACAGGTCTTTATCATATTGGATACTTCGGC |
| | GGGATTTTCTTTATCATCCTGTTGACTATTGACCGATACCTTGCTATCGTACACG |
| | CAGTTTTCGCCCTTAAGGCGAGGACAGTCACCTTTGGTGTAGTGACATCCGTCAT |
| | CACTTGGTTAGTGGCCGTTTTCGCTAGCGTGCCCGGGATTATCTTTACTAAATGC |
| | CAGAAAGAGGATTCCGTGTATGTATGTGGGCCCTACTTCCCGAGGGGCTGGAATA |
| | ACTTTCACACAATTATGCGAAATATCCTAGGCCTTGTCTTGCCTCTTCTGATCAT |
| | GGTGATATGTTATTCTGGAATCCTGAAAACATTACTCAGATGCCGCAATGAAAAG |
| | AAGAGGCACCGGGCCGTGCGCGTCATTTTTACCATCATGATTGTGTACTTCCTAT |
| | TTTGGACGCCGTACAATATCGTGATTTTGTTGAACACCTTCCAGGAGTTTTTTGG |
| | ACTGTCTAACTGCGAAAGCACTTCACAGTTAGACCAGGCGACTCAAGTGACGGAG |
| | ACACTGGGCATGACCCACTGCTGTATAAATCCCATAATATACGCCTTCGTAGGCG |
| | AGAAGTTTCGTAGTCTGTTTCACATTGCCTTGGGGTGTCGCATCGCACCTCTCCA |
| | AAAGCCAGTTTGCGGGGGACCAGGCGTCAGACCTGGCAAAAATGTTAAGGTCACC |
| | ACCCAGGGCCTGCTCGACGGACGGGGGAAGGGTAAAAGCATCGGAAGGGCACCCG |
| | AAGCTTCCCTGCAAGACAAGGAGGGCGCTTAGAACGCCATgggtggcatccctgt |
| | gacccctccccagtgcctctcctggccctggaagttgccactccagtgcccacca |
| | gccttgtcctaataaaattaagttgcatcattttgtctgactaggtgtccttcta |
| | taatattatggggtggaggggggtggtatggagcaaggggcaagttgggaagaga |
| | acctgtagggcctgcggggtctattgggaaccaagctggagtgcagtggcacaat |
| | cttggctcactgcaatctccgcctcctgggttcaagcgattctcctgcctcagcc |
| | tcccgagttgttgggattccaggcatgcatgaccaggctcagctaatttttgttt |
| | ttttggtagagTcggggtttcaccatattggccaggctggtgtccaactcctaat |
| | ctcaggtgatctacccaccttggcctcccaaattgctgggattacaggcgtgaac |
| | cactgctcccttccctgtccttctgattttaaaataactataccagcaggaggac |
| | gtccagacacagcataggctacctggccatgcccaaccggtgggacatttgagtt |
| | gcttgcttggcactgtcctctcatgcgttgggtccactcagtagatgcctgttCA |
| | GTCAGTTCGCtcttttgcaatgctctttgggaaattatattagcctaattactaa |
| | tttcctggccctcaaggtgacttgcttgaacttgccacctTCGCCACTAGGGGGC |
| | AGCAttggtttacacagggtgaacaaacgttcaattctaaactaatattctttgg |
| | tgggaaagtgtgttcattttggtttgttttgaagaagtcacttcatacctttgaa |
| | ctggggttaagctggttaactcccaaaattccaccagttccaatatcctatggaa |
| | aaaccccaaaaccactCAAAggatctgcgatcgctccggtgcccgtcagtgggca |
| | gagcgcacatcgcccacagtccccgagaagttggggggaggggtcggcaattgaa |
| | cgggtgcctagagaaggtggcgcggggtaaactgggaaagtgatgtcgtgtactg |
| | gctccgcctttttcccgagggtgggggagaaccgtatataagtgcagtagtcgcc |
| | gtgaacgttctttttcgcaacgggtttgccgccagaacacagctgaagcttcgag |
| | gggctcgcatctctccttcacgcgcccgccgccctacctgaggccgccatccacg |
| | ccggttgagtcgcgttctgccgcctcccgcctgtggtgcctcctgaactgcgtcc |
| | gccgtctaggtaagtttaaagctcaggtcgagTccgggcctttgtccggcgctcc |
| | cttggagcctacctagactcagccggctctccacgctttgcctgaccctgcttgc |
| | tcaactctacgtctttgtttcgttttctgttctgcgccgttacagatccaagctg |
| | tgaccggcgcctacGAATGCCGCCACCATGAAAGATAGCTGTATCACTGTCATGG |
| | CCATGGCCCTGTTGAGCGGCTTCTTCTTTTTTGCTCCAGCATCCTCCTATAACCT |
| | GGACGTTCGCGGGGCACGGTCCTTTTCCCCTCCTAGGGCCGGCAGGCATTTTGGC |
| | TACCGTGTGCTGCAGGTCGGTAACGGAGTAATCGTGGGTGCCCCGGGAGAAGGAA |
| | ACAGCACGGGAAGTCTGTACCAGTGCCAGTCTGGTACCGGCCACTGCTTACCCGT |
| | CACCCTACGCGGGAGTAACTACACATCCAAATACCTCGGAATGACTCTTGCAACA |
| | GATCCGACCGATGGGAGCATTTTGGCCTGTGATCCAGGCCTGTCTCGAACCTGCG |
| | ACCAAAACACATACCTGTCCGGACTCTGTTACCTCTTTCGCCAGAATTTGCAGGG |
| | GCCGATGCTGCAGGGCCGGCCCGGATTTCAGGAGTGTATAAAAGGCAATGTGGAC |
| | CTAGTATTTCTTTTTGACGGAAGCATGAGCCTGCAACCGGATGAGTTTCAGAAGA |
| | TCCTAGACTTTATGAAAGATGTTATGAAGAAGCTCTCCAACACTTCCTATCAGTT |
| | TGCCGCAGTGCAGTTCTCCACCTCCTATAAGACGGAGTTTGATTTCTCTGACTAT |
| | GTGAAACGCAAAGATCCCGATGCCCTGCTGAAGCATGTGAAGCATATGTTGTTGC |
| | TGACTAACACCTTTGGGGCTATAAACTATGTGGCCACCGAGGTGTTTAGGGAGGA |
| | ATTGGGTGCCCGCCCTGACGCCACCAAGGTCCTTATTATCATAACAGACGGCGAA |
| | GCCACAGACTCAGGCAACATTGATGCTGCAAAAGATATAATTCGATACATTATAG |
| | GAATCGGCAAGCACTTTCAGACAAAGGAGTCTCAAGAGACTCTCCATAAGTTTGC |
| | CAGTAAGCCTGCTTCAGAGTTCGTAAAAATTCTGGACACTTTCGAAAAGCTGAAA |
| | GACCTCTTTACAGAGCTCCAGAAGAAAATCTATGTCATCGAAGGCACTTCCAAGC |
| | AGGATCTGACTAGCTTCAACATGGAGCTCTCTTCAAGCGGCATTTCAGCTGATCT |
| | CTCTCGCGGTCACGCGGTCGTGGGGGCGGTAGGAGCCAAAGATTGGGCTGGTGGC |
| | TTCTTGGACCTGAAGGCTGACCTGCAAGACGACACATTCATCGGTAATGAACCCC |
| | TAACGCCCGAAGTGAGAGCCGGGTACTTGGGCTATACTGTCACCTGGTTGCCATC |
| | GAGACAGAAAACCTCTCTGTTAGCCAGCGGAGCGCCCAGATACCAACACATGGGT |
| | CGGGTTCTCTTGTTTCAGGAACCACAAGGCGGAGGTCACTGGAGCCAGGTGCAGA |
| | CTATCCATGGAACCCAGATCGGAAGCTACTTCGGCGGGGAATTATGTGGCGTTGA |
| | CGTGGACCAGGACGGGGAGACTGAACTGCTTCTGATCGGAGCACCCCTTTTCTAT |
| | GGAGAGCAGCGGGGAGGAAGAGTGTTCATCTACCAACGTAGACAGCTGGGTTTCG |
| | AAGAGGTTTCAGAGCTTCAAGGCGATCCAGGCTATCCCCTGGGGCGTTTTGGCGA |
| | GGCGATCACGGCTCTGACCGATATTAACGGCGACGGCCTGGTGGACGTTGCAGTT |
| | GGGGCTCCGCTGGAGGAACAGGGTGCCGTATACATATTCAACGGCAGACACGGAG |
| | GACTTTCTCCCCAGCCCTCCCAGAGAATCGAGGGAACACAGGTCCTGTCTGGCAT |
| | TCAGTGGTTCGGCCGGAGTATCCACGGAGTCAAGGATCTTGAAGGCGATGGGCTT |
| | GCAGACGTAGCCGTGGGGGCAGAAAGCCAGATGATCGTTTTGTCGTCAAGGCCTG |
| | TCGTTGACATGGTCACTCTGATGTCCTTTTCCCCCGCCGAAATACCAGTACACGA |
| | GGTGGAGTGCTCCTATTCGACTAGTAATAAGATGAAAGAGGGTGTAAATATCACG |
| | ATCTGCTTTCAGATTAAGTCTCTTATTCCACAGTTCCAGGGGCGCCTGGTGGCCA |
| | ATCTCACCTATACCTTACAGCTTGACGGGCACCGTACACGGCGGAGGGGTCTATT |
| | TCCAGGCGGGAGACACGAGCTCAGGAGGAATATAGCAGTGACAACAAGCATGAGT |
| | TGCACCGACTTCAGCTTCCACTTCCCTGTCTGCGTCCAGGATCTGATTAGTCCTA |
| | TCAATGTCTCTCTGAACTTCAGTCTTTGGGAGGAGGAAGGCACACCTAGAGATCA |
| | AAGGGCTCAGGGGAAGGATATACCTCCAATTTTGCGACCCTCCCTCCACAGCGAA |
| | ACATGGGAAATCCCATTCGAAAAGAACTGTGGCGAAGATAAGAAATGTGAGGCTA |
| | ATCTGCGAGTTTCATTTAGCCCCGCCAGGAGCCGCGCTCTGAGGCTTACCGCCTT |
| | CGCATCATTAAGTGTGGAACTATCCCTCAGCAATCTCGAGGAGGACGCCTACTGG |
| | GTGCAACTGGATCTCCATTTCCCTCCCGGACTCTCGTTTCGAAAGGTGGAAATGC |
| | TCAAGCCTCATAGTCAAATTCCAGTAAGCTGTGAGGAGCTCCCGGAGGAGTCTCG |
| | CCTGTTGTCTCGAGCATTAAGCTGCAATGTTAGCTCACCCATCTTCAAAGCCGGC |
| | CATTCAGTCGCACTGCAGATGATGTTCAACACCCTCGTGAACTCCAGCTGGGGTG |
| | ACTCCGTCGAATTACATGCAAATGTTACCTGCAATAACGAGGATAGCGATCTCCT |
| | CGAGGACAACTCCGCTACGACCATTATCCCTATTTTGTATCCCATCAATATTCTC |
| | ATCCAGGACCAAGAGGATTCAACACTGTACGTCAGCTTCACCCCAAAGGGTCCAA |
| | AGATTCACCAGGTCAAACATATGTATCAGGTGCGGATTCAACCTTCAATACACGA |
| | CCACAATATACCAACATTGGAGGCCGTAGTGGGGGTGCCACAGCCCCCTTCTGAG |
| | GGACCTATCACCCACCAGTGGTCCGTGCAGATGGAGCCACCCGTTCCATGCCATT |
| | ATGAAGATCTGGAGCGGCTACCTGACGCTGCCGAACCTTGTTTGCCTGGGGCGCT |
| | GTTTAGATGCCCCGTGGTGTTCCGGCAAGAAATTCTGGTGCAAGTGATCGGCACT |
| | TTAGAGCTTGTGGGGGAGATTGAAGCGTCTAGTATGTTCTCTCTGTGTAGTTCTC |
| | TGAGCATCTCATTCAATAGTTCTAAGCATTTTCACCTTTACGGGTCAAATGCGTC |
| | ACTGGCTCAAGTTGTGATGAAAGTGGACGTCGTGTACGAAAAACAGATGTTATAC |
| | CTGTACGTGCTGAGTGGTATCGGCGGACTGCTACTGCTGCTGCTTATTTTCATCG |
| | TGCTGTATAAGGTGGGATTCTTTAAAAGGAACTTAAAGGAAAAAATGGAAGCCGG |
| | GAGAGGGGTTCCGAACGGGATTCCCGCTGAGGACAGTGAGCAGCTCGCATCTGGT |
| | CAGGAGGCTGGGGACCCAGGGTGCCTCAAACCTCTGCATGAAAAAGATTCGGAGT |
| | CCGGCGGCGGCAAGGATTAAAACGCCATgggtggcatccctgtgacccctcccca |
| | gtgcctctcctggccctggaagttgccactccagtgcccaccagccttgtcctaa |
| | taaaattaagttgcatcattttgtctgactaggtgtccttctataatattatggg |
| | gtggaggggggtggtatggagcaaggggcaagttgggaagagaacctgtagggcc |
| | tgcggggtctattgggaaccaagctggagtgcagtggcacaatcttggctcactg |
| | caatctccgcctcctgggttcaagcgattctcctgcctcagcctcccgagttgtt |
| | gggattccaggcatgcatgaccaggctcagctaatttttgtttttttggtagagT |
| | cggggtttcaccatattggccaggctggtgtccaactcctaatctcaggtgatct |
| | acccaccttggcctcccaaattgctgggattacaggcgtgaaccactgctccctt |
| | ccctgtccttCAGTTAGGTCGCtcttttgcaatgctctttgggaaattatattag |
| | cctaattactaatttcctggccctcaaggtgacttgcttgaacttgccacctTCG |
| | CCACTAGGGGGCAGCAttggtttacacagggtgaacaaacgttcaattctaaact |
| | aatattctttggtgggaaagtgtgttcattttggtttgttttgaagaagtcactt |
| | catacctttgaactggggttaagctggttaactcccaaaattccaccagttccaa |
| | tatcctatggaaaaaccccaaaaccactCAAAggatctgcgatcgctccggtgcc |
| | cgtcagtgggcagagcgcacatcgcccacagtccccgagaagttggggggagggg |
| | tcggcaattgaacgggtgcctagagaaggtggcgcggggtaaactgggaaagtga |
| | tgtcgtgtactggctccgcctttttcccgaggggggggagaaccgtatataagtg |
| | cagtagtcgccgtgaacgttctttttcgcaacgggtttgccgccagaacacagct |
| | gaagcttcgaggggctcgcatctctccttcacgcgcccgccgccctacctgaggc |
| | cgccatccacgccggttgagtcgcgttctgccgcctcccgcctgtggtgcctcct |
| | gaactgcgtccgccgtctaggtaagtttaaagctcaggtcgagTccgggcctttg |
| | tccggcgctcccttggagcctacctagactcagccggctctccacgctttgcctg |
| | accctgcttgctcaactctacgtctttgtttcgttttctgttctgcgccgttaca |
| | gatccaagctgtgaccggcgcctacGAATGCCGCCACCATGTTAGGTCTGCGCCC |
| | TCCACTGCTGGCCCTGGTAGGATTACTCTCCTTGGGGTGTGTTCTCAGCCAGGAG |
| | TGCACCAAATTTAAAGTCTCCAGCTGCCGGGAGTGTATCGAGAGCGGCCCAGGCT |
| | GCACATGGTGCCAAAAACTCAACTTCACCGGACCAGGAGATCCCGACAGTATAAG |
| | ATGTGATACTAGGCCTCAGCTGTTGATGCGGGGCTGTGCTGCCGACGACATTATG |
| | GACCCTACCAGCTTAGCAGAAACCCAGGAAGATCATAATGGGGGGCAAAAACAGC |
| | TCAGCCCCCAGAAAGTGACCCTGTACCTGCGACCCGGTCAAGCGGCTGCCTTCAA |
| | CGTGACTTTTCGGCGCGCGAAGGGATACCCCATCGATCTTTATTATCTGATGGAT |
| | TTGAGCTACTCAATGCTGGATGACCTGAGGAACGTAAAGAAGTTGGGAGGGGATC |
| | TGCTGCGCGCTCTTAATGAAATTACAGAATCAGGCAGGATCGGCTTCGGATCATT |
| | CGTGGACAAAACAGTGCTGCCGTTCGTGAACACACACCCGGACAAGCTAAGAAAT |
| | CCTTGCCCCAACAAGGAAAAGGAGTGCCAGCCGCCATTTGCTTTCCGACACGTGC |
| | TGAAACTCACGAATAACTCAAATCAATTCCAAACCGAAGTGGGCAAGCAGCTGAT |
| | CTCTGGCAACCTGGACGCCCCCGAGGGGGGACTAGATGCGATGATGCAAGTTGCC |
| | GCTTGCCCCGAAGAGATTGGCTGGCGCAACGTGACCAGACTGCTGGTCTTTGCAA |
| | CTGATGACGGCTTCCATTTTGCTGGCGATGGAAAGTTGGGGGCAATCCTGACACC |
| | TAATGACGGCCGCTGCCATCTAGAAGATAACCTCTATAAGAGGAGTAATGAGTTT |
| | GACTATCCAAGTGTCGGCCAGCTGGCACATAAACTAGCCGAGAACAACATCCAAC |
| | CGATCTTCGCAGTCACAAGTAGGATGGTAAAGACGTACGAGAAGCTGACTGAGAT |
| | CATACCAAAATCTGCAGTGGGGGAGCTGAGCGAAGATTCTAGCAATGTTGTGCAG |
| | CTTATTAAGAATGCCTATAATAAGCTTTCCAGTAGAGTGTTTCTGGACCACAATG |
| | CCCTTCCCGACACTCTGAAAGTTACTTACGATTCTTTTTGTTCCAACGGCGTGAC |
| | GCACCGGAATCAGCCTCGCGGTGACTGTGACGGCGTGCAGATCAACGTGCCTATT |
| | ACATTTCAGGTTAAAGTCACTGCCACCGAGTGCATCCAGGAGCAATCATTTGTCA |
| | TCAGGGCTCTCGGTTTCACGGACATTGTGACCGTTCAGGTGCTCCCTCAGTGCGA |
| | ATGTCGATGCAGAGATCAGAGCAGAGATAGGTCCTTATGTCACGGGAAGGGTTTC |
| | TTGGAATGTGGGATATGCAGATGCGATACAGGCTATATAGGGAAGAACTGCGAAT |
| | GCCAGACACAGGGCCGATCGTCCCAGGAGCTGGAGGGGAGCTGTCGCAAAGATAA |
| | CAACTCTATAATTTGCAGCGGGCTCGGTGACTGTGTGTGTGGTCAGTGTCTCTGC |
| | CACACTAGTGACGTACCAGGGAAACTCATTTATGGCCAGTACTGCGAGTGTGATA |
| | CCATTAATTGTGAGAGGTATAACGGACAGGTCTGCGGCGGGCCAGGCCGTGGACT |
| | TTGTTTCTGCGGTAAGTGCCGATGCCATCCTGGATTTGAAGGCAGTGCTTGTCAA |
| | TGCGAGCGTACCACTGAGGGCTGTTTAAACCCTCGGCGTGTCGAGTGCTCGGGAC |
| | GCGGTAGATGCCGGTGTAACGTCTGCGAGTGCCACTCTGGTTACCAGCTCCCCCT |
| | CTGTCAGGAATGTCCCGGATGCCCTTCTCCTTGCGGGAAGTATATTTCCTGTGCA |
| | GAATGTCTGAAGTTTGAGAAGGGCCCCTTCGGGAAGAACTGCTCCGCCGCGTGCC |
| | CAGGACTTCAGTTATCAAATAATCCAGTAAAAGGGAGAACCTGTAAGGAAAGGGA |
| | TTCCGAAGGCTGTTGGGTGGCTTACACACTGGAGCAGCAAGACGGTATGGACAGA |
| | TACCTTATCTACGTCGACGAGTCTCGGGAGTGTGTGGCCGGACCAAATATCGCTG |
| | CCATCGTCGGCGGAACAGTGGCCGGAATTGTTCTGATCGGGATACTTCTACTCGT |
| | TATCTGGAAAGCATTGATTCACTTGTCTGACCTGCGTGAATACCGGCGGTTCGAG |
| | AAAGAAAAGTTGAAATCGCAGTGGAATAATGACAATCCCCTCTTTAAATCAGCCA |
| | CCACTACCGTGATGAACCCCAAGTTCGCCGAGTCCTAAAACGCCATgaattcact |
| | cctcaggtgcaggctgcctatcagaaggtggtggctggtgtggccaatgccctgg |
| | ctcacaaataccactgagatctttttccctctgccaaaaattatggggacatcat |
| | gaagccccttgagcatctgacttctggctaataaaggaaatttattttcattgca |
| | atagtgtgttggaattttttgtgtctctcactcggaaggacatatgggagggcaa |
| | atcatttaaaacatcagaatgagtatttggtttagagtttggcaacatatgccca |
| | tatgctggctgccatgaacaaaggttggctataaagaggtcatcagtatatgaaa |
| | cagccccctgctgtccattccttattccatagaaaagccttgacttgaggttaga |
| | ttttttttatattttgttttgtgttatttttttctttaacatccctaaaattttc |
| | cttacatgttttactagccagatttttcctcctctcctgactactcccagtcata |
| | gctgtccctcttctcttatggagatCAGTGAATGCGGCCGCTCGCtcttttgcaa |
| | tgctctttgggaaattatattagcctaattactaatttcctggccctcaaggtga |
| | cttgcttgaacttgccacctTCGCCACTAGGGGGCAGCAttggtttacacagggt |
| | gaacaaacgttcaattctaaactaatattctttggtgggaaagtgtgttcatttt |
| | ggtttgttttgaagaagtcacttcatacctttgaactggggttaagctggttaac |
| | tcccaaaattccaccagttccaatatcctatggaaaaaccccaaaaccactCAAA |
| | ggatctgcgatcgctccggtgcccgtcagtgggcagagcgcacatcgcccacagt |
| | ccccgagaagttggggggaggggtcggcaattgaacgggtgcctagagaaggtgg |
| | cgcggggtaaactgggaaagtgatgtcgtgtactggctccgcctttttcccgagg |
| | ggggggagaaccgtatataagtgcagtagtcgccgtgaacgttctttttcgcaac |
| | gggtttgccgccagaacacagctgaagcttcgaggggctcgcatctctccttcac |
| | gcgcccgccgccctacctgaggccgccatccacgccggttgagtcgcgttctgcc |
| | gcctcccgcctgtggtgcctcctgaactgcgtccgccgtctaggtaagtttaaag |
| | ctcaggtcgagTccgggcctttgtccggcgctcccttggagcctacctagactca |
| | gccggctctccacgctttgcctgaccctgcttgctcaactctacgtctttgtttc |
| | gttttctgttctgcgccgttacagatccaagctgtgaccggcgcctacGAATGCC |
| | GCCACCATGCTGTCAACATCCCGGTCTAGATTCATAAGAAACACCAATGAGAGTG |
| | GCGAAGAGGTGACCACTTTCTTCGATTATGATTATGGAGCCCCCTGTCATAAATT |
| | TGACGTCAAACAGATCGGTGCACAGCTGCTGCCACCTCTTTATAGCCTCGTGTTT |
| | ATTTTCGGGTTCGTGGGGAATATGCTCGTGGTTTTGATCCTCATCAACTGCAAGA |
| | AGCTCAAATGTCTGACCGATATATACCTACTGAACCTGGCCATTTCAGATCTGCT |
| | CTTCCTGATTACGCTGCCACTCTGGGCACATTCGGCCGCTAACGAATGGGTGTTT |
| | GGTAACGCTATGTGCAAGCTGTTCACAGGTCTTTATCATATTGGATACTTCGGCG |
| | GGATTTTCTTTATCATCCTGTTGACTATTGACCGATACCTTGCTATCGTACACGC |
| | AGTTTTCGCCCTTAAGGCGAGGACAGTCACCTTTGGTGTAGTGACATCCGTCATC |
| | ACTTGGTTAGTGGCCGTTTTCGCTAGCGTGCCCGGGATTATCTTTACTAAATGCC |
| | AGAAAGAGGATTCCGTGTATGTATGTGGGCCCTACTTCCCGAGGGGCTGGAATAA |
| | CTTTCACACAATTATGCGAAATATCCTAGGCCTTGTCTTGCCTCTTCTGATCATG |
| | GTGATATGTTATTCTGGAATCCTGAAAACATTACTCAGATGCCGCAATGAAAAGA |
| | AGAGGCACCGGGCCGTGCGCGTCATTTTTACCATCATGATTGTGTACTTCCTATT |
| | TTGGACGCCGTACAATATCGTGATTTTGTTGAACACCTTCCAGGAGTTTTTTGGA |
| | CTGTCTAACTGCGAAAGCACTTCACAGTTAGACCAGGCGACTCAAGTGACGGAGA |
| | CACTGGGCATGACCCACTGCTGTATAAATCCCATAATATACGCCTTCGTAGGCGA |
| | GAAGTTTCGTAGTCTGTTTCACATTGCCTTGGGGTGTCGCATCGCACCTCTCCAA |
| | AAGCCAGTTTGCGGGGGACCAGGCGTCAGACCTGGCAAAAATGTTAAGGTCACCA |
| | CCCAGGGCCTGCTCGACGGACGGGGGAAGGGTAAAAGCATCGGAAGGGCACCCGA |
| | AGCTTCCCTGCAAGACAAGGAGGGCGCTTAGAACGCCATgggtggcatccctgtg |
| | acccctccccagtgcctctcctggccctggaagttgccactccagtgcccaccag |
| | ccttgtcctaataaaattaagttgcatcattttgtctgactaggtgtccttctat |
| | aatattatggggtggaggggggggtatggagcaaggggcaagttgggaagagaac |
| | ctgtagggcctgcggggtctattgggaaccaagctggagtgcagtggcacaatct |
| | tggctcactgcaatctccgcctcctgggttcaagcgattctcctgcctcagcctc |
| | ccgagttgttgggattccaggcatgcatgaccaggctcagctaatttttgttttt |
| | ttggtagagTcggggtttcaccatattggccaggctggtgtccaactcctaatct |
| | caggtgatctacccaccttggcctcccaaattgctgggattacaggcgtgaacca |
| | ctgctcccttccctgtccttctgattttaaaataactataccagcaggaggacgt |
| | ccagacacagcataggctacctggccatgcccaaccggtgggacatttgagttgc |
| | ttgcttggcactgtcctctcatgcgttgggtccactcagtagatgcctgttCAGT |
| | GGATCCTCGCtcttttgcaatgctctttgggaaattatattagcctaattactaa |
| | tttcctggccctcaaggtgacttgcttgaacttgccacctTCGCCACTAGGGGGC |
| | AGCAttggtttacacagggtgaacaaacgttcaattctaaactaatattctttgg |
| | tgggaaagtgtgttcattttggtttgttttgaagaagtcacttcatacctttgaa |
| | ctggggttaagctggttaactcccaaaattccaccagttccaatatcctatggaa |
| | aaaccccaaaaccactCAAAggatctgcCatcgctccggtgcccgtcagtgggca |
| | gagcgcacatcgcccacagtccccgagaagttggggggaggggtcggcaattgaa |
| | cgggtgcctagagaaggtggcgcggggtaaactgggaaagtgatgtcgtgtactg |
| | gctccgcctttttcccgaggggggggagaaccgtatataagtgcagtagtcgccg |
| | tgaacgttctttttcgcaacgggtttgccgccagaacacagctgaagcttcgagg |
| | ggctcgcatctctccttcacgcgcccgccgccctacctgaggccgccatccacgc |
| | cggttgagtcgcgttctgccgcctcccgcctgtggtgcctcctgaactgcgtccg |
| | ccgtctaggtaagtttaaagctcaggtcgagTccgggcctttgtccggcgctccc |
| | ttggagcctacctagactcagccggctctccacgctttgcctgaccctgcttgct |
| | caactctacgtctttgtttcgttttctgttctgcgccgttacagatccaagctgt |
| | gaccggcgcctacGAATGCCGCCACCATGGCTgctgaaggTtccgtcgccaggca |
| | gcctgacctcttgacctgcgacgatgagccgatccatatccccggtgccatccaa |
| | ccgcatggactgctgctcgccctcgccgccgacatgacgatcgttgccggcagcg |
| | acaaccttcccgaactcaccggactggcgatcggcgccctgatcggccgctctgc |
| | ggccgatgtGttcgactcggaAacgcacaaccgtctgacgatcgccttggccgag |
| | cccggggcggccgtcggagcaccgatcactgtcggcttcacgatgcgaaaggacg |
| | caggcttcatcggctcctggcatcgccatgatcagctcatcttcctGgagctGga |
| | gcctccccagcgggacgtcgccgagccgcagggttcttccgccgcaccaacagcg |
| | ccatccgccgcctgcaggccgccgaaaccttggaaagcgcctgcgccgccgcggc |
| | gcaagaggtgcggaagattaccggcttcgatcgggtgatgatctatcgcttcgcc |
| | tccgacttcagcggcgaagtgatcgcagaggatcggtgcgccgaggtcgagtcaa |
| | aactaggcctgcactatcctgcctcaaccgtgccggcgcaggcccgtcggctcta |
| | taccatcaacccggtacggatcattcccgatatcaattatcggccggtgccggtc |
| | accccagacctcaatccggtcaccgggcggccgattgatcttagcttcgccatcc |
| | tgcgcagcgtGtcgcccgtccatctggaGttcatgcgcaacataggcatgcacgg |
| | cacgatgtcgatctcgattttgcgcggcgagcgactgtggggattgatcgtttgc |
| | catcaccgaacgccgtactacgtcgatctcgatggccgccaagcctgcgagctag |
| | tcgcccaggttctggcctggcagatcggcgtgatggaagagTAAAACGBBABBAB |
| | BABBABBABBACCATctgtgccttctagttgccagccatctgttgtttgcccctc |
| | ccccgtgccttccttgaccctggaaggtgccactcccactgtcctttcctaataa |
| | aatgaggaaattgcatcgcattgtctgagtaggtgtcattctattctggggggtg |
| | gggggggcaggacagcaagggggaggattgggaaggcaatagcaggcatgctggg |
| | gatgcggtgggctctatggCAGTttaattaacgagagcataatattgatatgtgc |
| | caaagttgtttctgactgactaataagtataatttgtttctattatgtataggtt |
| | aagctaattacttattttataatacaacatgactgtttttaaagtacaaaataag |
| | tttatttttgtaaaagagagaatgtttaaaagttttgttactttatagaagaaat |
| | tttgagtttttgtttttttttaataaataaataaacataaataaattgtttgttg |
| | aatttattattagtatgtaagtgtaaatataataaaacttaatatctattcaaat |
| | taataaataaacctcgatatacagaccgataaaacacatgcgtcaattttacgca |
| | tgattatctttaacgtacgtcacaatatgattatctttctagggttaaataatag |
| | tttctaatttttttattattcagcctgctgtgtgaataccgagctccaattcgcc |
| | ctatagtgagtcgtattacaattcactggccgtcgttttacaacgtcgtgactgg |
| | gaaaaccctggcgttacccaacttaatcgccttgcagcacatccccctttcgcca |
| | gctggcgtaatagcgaagaggcccgcaccgatcgcccttcccaacagttgcgcag |
| | cctgaatggcgaatgggacgcgccctgtagcggcgcattaagcgcggcgggtgtg |
| | gtggttacgcgcagcgtgaccgctacacttgccagcgccctagcgcccgctcctt |
| | tcgctttcttcccttcctttctcgccacgttcgccggctttccccgtcaagctct |
| | aaatcgggggctccctttagggttccgatttagtgctttacggcacctcgacccc |
| | aaaaaacttgattagggtgatggttcacgtagtgggccatcgccctgatagacgg |
| | tttttcgccctttgacgttggagtccacgttctttaatagtggactcttgttcca |
| | aactggaacaacactcaaccctatctcggtctattcttttgatttataagggatt |
| | ttgccgatttcggcctattggttaaaaaatgagctgatttaacaaaaatttaacg |
| | cgaattttaacaaaatattaacgcttacaatttaggtggcacttttcggggaaat |
| | gtgcgcggaacccctatttgtttatttttctaaatacattcaaatatgtatccgc |
| | tcatgagacaataaccctgataaatgcttcaataatattgaaaaaggaagagtat |
| | gagtattcaacatttccgtgtcgcccttattcccttttttgcggcattttgcctt |
| | cctgtttttgctcacccagaaacgctggtgaaagtaaaagatgctgaagatcagt |
| | tgggtgcacgagtgggttacatcgaactggatctcaacagcggtaagatccttga |
| | gagttttcgccccgaagaacgttttccaatgatgagcacttttaaagttctgcta |
| | tgtggcgcggtattatcccgtattgacgccgggcaagagcaactcggtcgccgca |
| | tacactattctcagaatgacttggttgagtactcaccagtcacagaaaagcatct |
| | tacggatggcatgacagtaagagaattatgcagtgctgccataaccatgagtgat |
| | aacactgcggccaacttacttctgacaacgatcggaggaccgaaggagctaaccg |
| | cttttttgcacaacatgggggatcatgtaactcgccttgatcgttgggaaccgga |
| | gctgaatgaagccataccaaacgacgagcgtgacaccacgatgcctgtagcaatg |
| | gcaacaacgttgcgcaaactattaactggcgaactacttactctagcttcccggc |
| | aacaattaatagactggatggaggcggataaagttgcaggaccacttctgcgctc |
| | ggcccttccggctggctggtttattgctgataaatctggagccggtgagcgtggg |
| | tctcgcggtatcattgcagcactggggccagatggtaagccctcccgtatcgtag |
| | ttatctacacgacggggagtcaggcaactatggatgaacgaaatagacagatcgc |
| | tgagataggtgcctcactgattaagcattggtaactgtcagaccaagtttactca |
| | tatatactttagattgatttaaaacttcatttttaatttaaaaggatctaggtga |
| | agatcctttttgataatctcatgaccaaaatcccttaacgtgagttttcgttcca |
| | ctgagcgtcagaccccgtagaaaagatcaaaggatcttcttgagatccttttttt |
| | ctgcgcgtaatctgctgcttgcaaacaaaaaaaccaccgctaccagcggtggttt |
| | gtttgccggatcaagagctaccaactctttttccgaaggtaactggcttcagcag |
| | agcgcagataccaaatactgttcttctagtgtagccgtagttaggccaccacttc |
| | aagaactctgtagcaccgcctacatacctcgctctgctaatcctgttaccagtgg |
| | ctgctgccagtggcgataagtcgtgtcttaccgggttggactcaagacgatagtt |
| | accggataaggcgcagcggtcgggctgaacggggggttcgtgcacacagcccagc |
| | ttggagcgaacgacctacaccgaactgagatacctacagcgtgagctatgagaaa |
| | gcgccacgcttcccgaagggagaaaggcggacaggtatccggtaagcggcagggt |
| | cggaacaggagagcgcacgagggagcttccagggggaaacgcctggtatctttat |
| | agtcctgtcgggtttcgccacctctgacttgagcgtcgatttttgtgatgctcgt |
| | caggggggggagcctatggaaaaacgccagcaacgcggcctttttacggttcctg |
| | gccttttgctggccttttgctcacatgttctttcctgcgttatcccctgattctg |
| | tggataaccgtattaccgcctttgagtgagctgataccgctcgccgcagccgaac |
| | gaccgagcgcagcgagtcagtgagcgaggaagcggaagagcgcccaatacgcaaa |
| | ccgcctctccccgcgcgttggccgattcattaatgcagctggcacgacaggtttc |
| | ccgactggaaagcgggcagtgagcgcaacgcaattaatgtgagttagctcactca |
| | ttaggcaccccaggctttacactttatgcttccggctcgtatgttgtgtggaatt |
| | gtgagcggataacaatttcacacaggaaacagctatgaccatgattacgccaagc |
| | tcgaaattaaccctcactaaagggaacaaaagctggtacctcgcgcgacttggtt |
| | tgccattctttagcgcgcgtcgcgtcacacagcttggccacaatgtggtttttgt |
| | caaacgaagattctatgacgtgtttaaagtttaggtcgagtaaagcgcaaatctt |
| | ttttaaccctagaaagatagtctgcgtaaaattgacgcatgcattcttgaaatat |
| | tgctctctctttctaaatagcgcgaatccgtcgctgtgcatttaggacatctcag |
| | tcgccgcttggagctcccgtgaggcgtgcttgtcaatgcggtaagtgtcactgat |
| | tttgaactataacgaccgcgtgagtcaaaatgacgcatgattatcttttacgtga |
| | cttttaagatttaactcatacgataattatattgttatttcatgttctacttacg |
| | tgataacttattatatatatattttcttgttatagatatcatcaactttgtatag |
| | aaaagttgctcgacattgattattgactagttattaatagtaatcaattacgggg |
| | tcattagttcatagcccatatatggagttccgcgttacataacttacggtaaatg |
| | gcccgcctggctgaccgcccaacgacccccgcccattgacgtcaataatgacgta |
| | tgttcccatagtaacgccaatagggactttccattgacgtcaatgggtggagtat |
| | ttacggtaaactgcccacttggcagtacatcaagtgtatcatatgccaagtacgc |
| | cccctattgacgtcaatgacggtaaatggcccgcctggcattatgcccagtacat |
| | gaccttatgggactttcctacttggcagtacatctacgtattagtcatcgctatt |
| | accatggtcgaggtgagccccacgttctgcttcactctccccatctcccccccct |
| | ccccacccccaattttgtatttatttattttttaattattttgtgcagcgatggg |
| | ggcggggggggggggggggcgcgcgccaggcggggggggcggggcgagggggggg |
| | cggggcgaggcggagaggtgcggcggcagccaatcagagcggcgcgctccgaaag |
| | tttccttttatggcgaggcggcggcggcggcggccctataaaaagcgaagcgcgc |
| | ggggggggagtcgctgcgcgctgccttcgccccgtgccccgctccgccgccgcct |
| | cgcgccgcccgccccggctctgactgaccgcgttactcccacaggtgagcgggcg |
| | ggacggcccttctcctccgggctgtaattagcgcttggtttaatgacggcttgtt |
| | tcttttctgtggctgcgtgaaagccttgaggggctccgggagggccctttgtgcg |
| | gggggagcggctcggggggtgcgtgcgtgtgtgtgtgcgtggggagcgccgcgtg |
| | cggctccgcgctgcccggcggctgtgagcgctgcgggcgcggcgcggggctttgt |
| | gcgctccgcagtgtgcgcgaggggagcgcggccgggggcggtgccccgcggtgcg |
| | gggggggctgcgaggggaacaaaggctgcgtgcggggtgtgtgcgtgggggggtg |
| | agcagggggtgtgggcgcgtcggtcgggctgcaaccccccctgcacccccctccc |
| | cgagttgctgagcacggcccggcttcgggtgcggggctccgtacggggcgtggcg |
| | cggggctcgccgtgccgggcggggggtggcggcaggtgggggtgccgggcggggc |
| | ggggccgcctcgggccggggagggctcgggggaggggcgcggcggcccccggagc |
| | gccggcggctgtcgaggcgcggcgagccgcagccattgccttttatggtaatcgt |
| | gcgagagggcgcagggacttcctttgtcccaaatctgtgcggagccgaaatctgg |
| | gaggcgccgccgcaccccctctagcgggcgcggggcgaagcggtgcggcgccggc |
| | aggaaggaaatgggcggggagggccttcgtgcgtcgccgcgccgccgtccccttc |
| | tccctctccagcctcggggctgtccgcggggggacggctgccttcgggggggacg |
| | gggcagggcggggttcggcttctggcgtgtgaccggcggctctagagcctctgct |
| | aaccatgttcatgccttcttctttttcctacagctcctgggcaacgtgctggtta |
| | ttgtgctgtctcatcattttggcaaagaattgcaagtttgtacaaaaaagcaggc |
| | tGAATGCCGCCACCATGGCTaccgagtataaaccgacggtccgacttgcgactcg |
| | ggatgacgttccacgcgccgtgaggactctggccgccgccttcgctgactacccg |
| | gccacacggcacacagttgaccccgacagacacatagagcgggtcacagagttgc |
| | aagaacttttcctgacgcgggttggActcgatattggaaaagtttgggttgccga |
| | cgatggtgccgcagttgcggtgtggactactccagaaagtgtagaagctggagcc |
| | gtattcgctgaaatagggccacggatggcagagctgtctggtagcaggctcgcag |
| | cgcagcagcagatggaaggCcttctcgctccgcatcggcctaaggaaccggcgtg |
| | gttcctggctacggtgggagtctcacctgaccatcaaggaaagggattgggaagt |
| | gctgtcgttcttccaggcgtggaagcggccgaacgagcaggcgtcccagccttct |
| | tggaAacgagcgcacccaggaatcttccattctacgagcgactgggtttcactgt |
| | gacagcagacgtagaagtccctgaagggcctcggacttggtgcatgaccagaaag |
| | cctggggcttgaAACGCCATgatccagacatgataagatacattgatgagtttgg |
| | acaaaccacaactagaatgcagtgaGGCGCGCCTGGATACTTCTGCCTGCAGGGG |
| | AGTCGCtcttttgcaatgctctttgggaaattatattagcctaattactaatttc |
| | ctggccctcaaggtgacttgcttgaacttgccacctTCGCCACTAGGGGGCAGCA |
| | ttggtttacacagggtgaacaaacgttcaattctaaactaatattctttggtggg |
| | aaagtgtgttcattttggtttgttttgaagaagtcacttcatacctttgaactgg |
| | ggttaagctggttaactcccaaaattccaccagttccaatatcctatggaaaaac |
| | cccaaaaccactCAAAggggttggggttgcgccttttccaaggcagccctgggtt |
| | tgcgcagggacgcggctgctctgggcgtggttccgggaaacgcagcggcgccgac |
| | cctgggTctGgcacattcttcacgtccgttcgcagcgtcacccggatcttcgccg |
| | ctacccttgtgggccccccggcgacgcttcctgctccgcccctaagtcgggaagg |
| | ttccttgcggttcgcggcgtgccggacgtgacaaacggaagccgcacgTctGact |
| | agtaccctcgcagacggacagcgccagggagcaatggcagcgcgccgaccgcgat |
| | gggctgtggccaatagcggctgctcagcAgggcgcgccgagagcagcggccggga |
| | aggggcggtgcgggaggcggggtgtggggcggtagtgtgggccctgttcctgccc |
| | gcgcggtgttccgcattctgcaagcctccggagcgcacgtcggcagtcggctccc |
| | tcgttgaccgaatcaccgacctctctccccagGAATGCCGCCACCATGAATAACG |
| | CCGGCCACGGACCAACTAGACGCTTGAGGGGACTCGGAGTTTTGGCTGGAGTCGC |
| | ACTCCTGGCCGCTCTGTGGCTGCTTTGGCTCCTTGGAAGCGCGCCAAGGGGCACA |
| | CCTGCACCACAACCTACCATAACAATTCTGGTGTGGCATTGGCCATTTACCGATC |
| | AACCACCCGAATTGCCGTCAGATACATGTACCAGGTATGGGATTGCACGGTGTCA |
| | TCTCAGCGCGAATCGCTCATTGTTGGCTTC |
| | (SEQ ID NO: 42) |
| pAJW_P159 (IntaL | ttttttaaccctagaaagatagtctgcgtaaaattgacgcatgcattcttgaaat |
| IntB2 + CCR2) | attgctctctctttctaaatagcgcgaatccgtcgctgtgcatttaggacatctc |
| | agtcgccgcttggagctcccgtgaggcgtgcttgtcaatgcggtaagtgtcactg |
| | attttgaactataacgaccgcgtgagtcaaaatgacgcatgattatcttttacgt |
| | gacttttaagatttaactcatacgataattatattgttatttcatgttctactta |
| | cgtgataacttattatatatatattttcttgttatagatatcatcaactttgtat |
| | agaaaagttgctcgacattgattattgactagttattaatagtaatcaattacgg |
| | ggtcattagttcatagcccatatatggagttccgcgttacataacttacggtaaa |
| | tggcccgcctggctgaccgcccaacgacccccgcccattgacgtcaataatgacg |
| | tatgttcccatagtaacgccaatagggactttccattgacgtcaatgggtggagt |
| | atttacggtaaactgcccacttggcagtacatcaagtgtatcatatgccaagtac |
| | gccccctattgacgtcaatgacggtaaatggcccgcctggcattatgcccagtac |
| | atgaccttatgggactttcctacttggcagtacatctacgtattagtcatcgcta |
| | ttaccatggtcgaggtgagccccacgttctgcttcactctccccatctccccccc |
| | ctccccacccccaattttgtatttatttattttttaattattttgtgcagcgatg |
| | ggggggggggggggggggggcgcgcgccaggcggggggggggggcgaggggcggg |
| | gggggcgaggcggagaggtgcggcggcagccaatcagagcggcgcgctccgaaag |
| | tttccttttatggcgaggcggcggcggcggcggccctataaaaagcgaagcgcgc |
| | ggcgggcgggagtcgctgcgcgctgccttcgccccgtgccccgctccgccgccgc |
| | ctcgcgccgcccgccccggctctgactgaccgcgttactcccacaggtgagcggg |
| | cgggacggcccttctcctccgggctgtaattagcgcttggtttaatgacggcttg |
| | tttcttttctgtggctgcgtgaaagccttgaggggctccgggagggccctttgtg |
| | cggggggagcggctcggggggtgcgtgcgtgtgtgtgtgcgtggggagcgccgcg |
| | tgcggctccgcgctgcccggcggctgtgagcgctgcgggcgcggcgcggggcttt |
| | gtgcgctccgcagtgtgcgcgaggggagcgcggccgggggcggtgccccgcggtg |
| | cggggggggctgcgaggggaacaaaggctgcgtgcggggtgtgtgcgtggggggg |
| | tgagcagggggtgtgggcgcgtcggtcgggctgcaaccccccctgcacccccctc |
| | cccgagttgctgagcacggcccggcttcgggtgcggggctccgtacggggcgtgg |
| | cgcggggctcgccgtgccgggcggggggtggcggcaggtgggggtgccggggggg |
| | cggggccgcctcgggccggggagggctcgggggaggggcgcggcggcccccggag |
| | cgccggcggctgtcgaggcgcggcgagccgcagccattgccttttatggtaatcg |
| | tgcgagagggcgcagggacttcctttgtcccaaatctgtgcggagccgaaatctg |
| | ggaggcgccgccgcaccccctctagcgggcgcggggcgaagcggtgcggcgccgg |
| | caggaaggaaatgggcggggagggccttcgtgcgtcgccgcgccgccgtcccctt |
| | ctccctctccagcctcggggctgtccgcggggggacggctgccttcgggggggac |
| | ggggcagggcggggttcggcttctggcgtgtgaccggcggctctagagcctctgc |
| | taaccatgttcatgccttcttctttttcctacagctcctgggcaacgtgctggtt |
| | attgtgctgtctcatcattttggcaaagaattgcaagtttgtacaaaaaagcagg |
| | ctGAATGCCGCCACCATGGCTaccgagtataaaccgacggtccgacttgcgactc |
| | gggatgacgttccacgcgccgtgaggactctggccgccgccttcgctgactaccc |
| | ggccacacggcacacagttgaccccgacagacacatagagcgggtcacagagttg |
| | caagaacttttcctgacgcgggttggActcgatattggaaaagtttgggttgccg |
| | acgatggtgccgcagttgcggtgtggactactccagaaagtgtagaagctggagc |
| | cgtattcgctgaaatagggccacggatggcagagctgtctggtagcaggctcgca |
| | gcgcagcagcagatggaaggCcttctcgctccgcatcggcctaaggaaccggcgt |
| | ggttcctggctacggtgggagtctcacctgaccatcaaggaaagggattgggaag |
| | tgctgtcgttcttccaggcgtggaagcggccgaacgagcaggcgtcccagccttc |
| | ttggaAacgagcgcacccaggaatcttccattctacgagcgactgggtttcactg |
| | tgacagcagacgtagaagtccctgaagggcctcggacttggtgcatgaccagaaa |
| | gcctggggcttgaAACGCCATgatccagacatgataagatacattgatgagtttg |
| | gacaaaccacaactagaatgcagtgaGGCGCGCCTGGATACTTCTGCCTGCAGGG |
| | GAGTCGCtcttttgcaatgctctttgggaaattatattagcctaattactaattt |
| | cctggccctcaaggtgacttgcttgaacttgccacctTCGCCACTAGGGGGCAGC |
| | Attggtttacacagggtgaacaaacgttcaattctaaactaatattctttggtgg |
| | gaaagtgtgttcattttggtttgttttgaagaagtcacttcatacctttgaactg |
| | gggttaagctggttaactcccaaaattccaccagttccaatatcctatggaaaaa |
| | ccccaaaaccactCAAAggatctgcgatcgctccggtgcccgtcagtgggcagag |
| | cgcacatcgcccacagtccccgagaagttggggggaggggtcggcaattgaacgg |
| | gtgcctagagaaggtggcgcggggtaaactgggaaagtgatgtcgtgtactggct |
| | ccgcctttttcccgaggggggggagaaccgtatataagtgcagtagtcgccgtga |
| | acgttctttttcgcaacgggtttgccgccagaacacagctgaagcttcgaggggc |
| | tcgcatctctccttcacgcgcccgccgccctacctgaggccgccatccacgccgg |
| | ttgagtcgcgttctgccgcctcccgcctgtggtgcctcctgaactgcgtccgccg |
| | tctaggtaagtttaaagctcaggtcgagTccgggcctttgtccggcgctcccttg |
| | gagcctacctagactcagccggctctccacgctttgcctgaccctgcttgctcaa |
| | ctctacgtctttgtttcgttttctgttctgcgccgttacagatccaagctgtgac |
| | cggcgcctacGAATGCCGCCACCATGAAAGATAGCTGTATCACTGTCATGGCCAT |
| | GGCCCTGTTGAGCGGCTTCTTCTTTTTTGCTCCAGCATCCTCCTATAACCTGGAC |
| | GTTCGCGGGGCACGGTCCTTTTCCCCTCCTAGGGCCGGCAGGCATTTTGGCTACC |
| | GTGTGCTGCAGGTCGGTAACGGAGTAATCGTGGGTGCCCCGGGAGAAGGAAACAG |
| | CACGGGAAGTCTGTACCAGTGCCAGTCTGGTACCGGCCACTGCTTACCCGTCACC |
| | CTACGCGGGAGTAACTACACATCCAAATACCTCGGAATGACTCTTGCAACAGATC |
| | CGACCGATGGGAGCATTTTGGCCTGTGATCCAGGCCTGTCTCGAACCTGCGACCA |
| | AAACACATACCTGTCCGGACTCTGTTACCTCTTTCGCCAGAATTTGCAGGGGCCG |
| | ATGCTGCAGGGCCGGCCCGGATTTCAGGAGTGTATAAAAGGCAATGTGGACCTAG |
| | TATTTCTTTTTGACGGAAGCATGAGCCTGCAACCGGATGAGTTTCAGAAGATCCT |
| | AGACTTTATGAAAGATGTTATGAAGAAGCTCTCCAACACTTCCTATCAGTTTGCC |
| | GCAGTGCAGTTCTCCACCTCCTATAAGACGGAGTTTGATTTCTCTGACTATGTGA |
| | AACGCAAAGATCCCGATGCCCTGCTGAAGCATGTGAAGCATATGTTGTTGCTGAC |
| | TAACACCTTTGGGGCTATAAACTATGTGGCCACCGAGGTGTTTAGGGAGGAATTG |
| | GGTGCCCGCCCTGACGCCACCAAGGTCCTTATTATCATAACAGACGGCGAAGCCA |
| | CAGACTCAGGCAACATTGATGCTGCAAAAGATATAATTCGATACATTATAGGAAT |
| | CGGCAAGCACTTTCAGACAAAGGAGTCTCAAGAGACTCTCCATAAGTTTGCCAGT |
| | AAGCCTGCTTCAGAGTTCGTAAAAATTCTGGACACTTTCGAAAAGCTGAAAGACC |
| | TCTTTACAGAGCTCCAGAAGAAAATCTATGTCATCGAAGGCACTTCCAAGCAGGA |
| | TCTGACTAGCTTCAACATGGAGCTCTCTTCAAGCGGCATTTCAGCTGATCTCTCT |
| | CGCGGTCACGCGGTCGTGGGGGCGGTAGGAGCCAAAGATTGGGCTGGTGGCTTCT |
| | TGGACCTGAAGGCTGACCTGCAAGACGACACATTCATCGGTAATGAACCCCTAAC |
| | GCCCGAAGTGAGAGCCGGGTACTTGGGCTATACTGTCACCTGGTTGCCATCGAGA |
| | CAGAAAACCTCTCTGTTAGCCAGCGGAGCGCCCAGATACCAACACATGGGTCGGG |
| | TTCTCTTGTTTCAGGAACCACAAGGCGGAGGTCACTGGAGCCAGGTGCAGACTAT |
| | CCATGGAACCCAGATCGGAAGCTACTTCGGCGGGGAATTATGTGGCGTTGACGTG |
| | GACCAGGACGGGGAGACTGAACTGCTTCTGATCGGAGCACCCCTTTTCTATGGAG |
| | AGCAGCGGGGAGGAAGAGTGTTCATCTACCAACGTAGACAGCTGGGTTTCGAAGA |
| | GGTTTCAGAGCTTCAAGGCGATCCAGGCTATCCCCTGGGGCGTTTTGGCGAGGCG |
| | ATCACGGCTCTGACCGATATTAACGGCGACGGCCTGGTGGACGTTGCAGTTGGGG |
| | CTCCGCTGGAGGAACAGGGTGCCGTATACATATTCAACGGCAGACACGGAGGACT |
| | TTCTCCCCAGCCCTCCCAGAGAATCGAGGGAACACAGGTCCTGTCTGGCATTCAG |
| | TGGTTCGGCCGGAGTATCCACGGAGTCAAGGATCTTGAAGGCGATGGGCTTGCAG |
| | ACGTAGCCGTGGGGGCAGAAAGCCAGATGATCGTTTTGTCGTCAAGGCCTGTCGT |
| | TGACATGGTCACTCTGATGTCCTTTTCCCCCGCCGAAATACCAGTACACGAGGTG |
| | GAGTGCTCCTATTCGACTAGTAATAAGATGAAAGAGGGTGTAAATATCACGATCT |
| | GCTTTCAGATTAAGTCTCTTATTCCACAGTTCCAGGGGCGCCTGGTGGCCAATCT |
| | CACCTATACCTTACAGCTTGACGGGCACCGTACACGGCGGAGGGGTCTATTTCCA |
| | GGCGGGAGACACGAGCTCAGGAGGAATATAGCAGTGACAACAAGCATGAGTTGCA |
| | CCGACTTCAGCTTCCACTTCCCTGTCTGCGTCCAGGATCTGATTAGTCCTATCAA |
| | TGTCTCTCTGAACTTCAGTCTTTGGGAGGAGGAAGGCACACCTAGAGATCAAAGG |
| | GCTCAGGGGAAGGATATACCTCCAATTTTGCGACCCTCCCTCCACAGCGAAACAT |
| | GGGAAATCCCATTCGAAAAGAACTGTGGCGAAGATAAGAAATGTGAGGCTAATCT |
| | GCGAGTTTCATTTAGCCCCGCCAGGAGCCGCGCTCTGAGGCTTACCGCCTTCGCA |
| | TCATTAAGTGTGGAACTATCCCTCAGCAATCTCGAGGAGGACGCCTACTGGGTGC |
| | AACTGGATCTCCATTTCCCTCCCGGACTCTCGTTTCGAAAGGTGGAAATGCTCAA |
| | GCCTCATAGTCAAATTCCAGTAAGCTGTGAGGAGCTCCCGGAGGAGTCTCGCCTG |
| | TTGTCTCGAGCATTAAGCTGCAATGTTAGCTCACCCATCTTCAAAGCCGGCCATT |
| | CAGTCGCACTGCAGATGATGTTCAACACCCTCGTGAACTCCAGCTGGGGTGACTC |
| | CGTCGAATTACATGCAAATGTTACCTGCAATAACGAGGATAGCGATCTCCTCGAG |
| | GACAACTCCGCTACGACCATTATCCCTATTTTGTATCCCATCAATATTCTCATCC |
| | AGGACCAAGAGGATTCAACACTGTACGTCAGCTTCACCCCAAAGGGTCCAAAGAT |
| | TCACCAGGTCAAACATATGTATCAGGTGCGGATTCAACCTTCAATACACGACCAC |
| | AATATACCAACATTGGAGGCCGTAGTGGGGGTGCCACAGCCCCCTTCTGAGGGAC |
| | CTATCACCCACCAGTGGTCCGTGCAGATGGAGCCACCCGTTCCATGCCATTATGA |
| | AGATCTGGAGCGGCTACCTGACGCTGCCGAACCTTGTTTGCCTGGGGCGCTGTTT |
| | AGATGCCCCGTGGTGTTCCGGCAAGAAATTCTGGTGCAAGTGATCGGCACTTTAG |
| | AGCTTGTGGGGGAGATTGAAGCGTCTAGTATGTTCTCTCTGTGTAGTTCTCTGAG |
| | CATCTCATTCAATAGTTCTAAGCATTTTCACCTTTACGGGTCAAATGCGTCACTG |
| | GCTCAAGTTGTGATGAAAGTGGACGTCGTGTACGAAAAACAGATGTTATACCTGT |
| | ACGTGCTGAGTGGTATCGGCGGACTGCTACTGCTGCTGCTTATTTTCATCGTGCT |
| | GTATAAGGTGGGATTCTTTAAAAGGAACTTAAAGGAAAAAATGGAAGCCGGGAGA |
| | GGGGTTCCGAACGGGATTCCCGCTGAGGACAGTGAGCAGCTCGCATCTGGTCAGG |
| | AGGCTGGGGACCCAGGGTGCCTCAAACCTCTGCATGAAAAAGATTCGGAGTCCGG |
| | CGGCGGCAAGGATTAAAACGCCATgggtggcatccctgtgacccctccccagtgc |
| | ctctcctggccctggaagttgccactccagtgcccaccagccttgtcctaataaa |
| | attaagttgcatcattttgtctgactaggtgtccttctataatattatggggtgg |
| | aggggggtggtatggagcaaggggcaagttgggaagagaacctgtagggcctgcg |
| | gggtctattgggaaccaagctggagtgcagtggcacaatcttggctcactgcaat |
| | ctccgcctcctgggttcaagcgattctcctgcctcagcctcccgagttgttggga |
| | ttccaggcatgcatgaccaggctcagctaatttttgtttttttggtagagTcggg |
| | gtttcaccatattggccaggctggtgtccaactcctaatctcaggtgatctaccc |
| | accttggcctcccaaattgctgggattacaggcgtgaaccactgctcccttccct |
| | gtccttCAGTGGCATCGCtcttttgcaatgctctttgggaaattatattagccta |
| | attactaatttcctggccctcaaggtgacttgcttgaacttgccacctTCGCCAC |
| | TAGGGGGCAGCAttggtttacacagggtgaacaaacgttcaattctaaactaata |
| | ttctttggtgggaaagtgtgttcattttggtttgttttgaagaagtcacttcata |
| | cctttgaactggggttaagctggttaactcccaaaattccaccagttccaatatc |
| | ctatggaaaaaccccaaaaccactCAAAggatctgcgatcgctccggtgcccgtc |
| | agtgggcagagcgcacatcgcccacagtccccgagaagttggggggaggggtcgg |
| | caattgaacgggtgcctagagaaggtggcgcggggtaaactgggaaagtgatgtc |
| | gtgtactggctccgcctttttcccgagggtgggggagaaccgtatataagtgcag |
| | tagtcgccgtgaacgttctttttcgcaacgggtttgccgccagaacacagctgaa |
| | gcttcgaggggctcgcatctctccttcacgcgcccgccgccctacctgaggccgc |
| | catccacgccggttgagtcgcgttctgccgcctcccgcctgtggtgcctcctgaa |
| | ctgcgtccgccgtctaggtaagtttaaagctcaggtcgagTccgggcctttgtcc |
| | ggcgctcccttggagcctacctagactcagccggctctccacgctttgcctgacc |
| | ctgcttgctcaactctacgtctttgtttcgttttctgttctgcgccgttacagat |
| | ccaagctgtgaccggcgcctacGAATGCCGCCACCATGTTAGGTCTGCGCCCTCC |
| | ACTGCTGGCCCTGGTAGGATTACTCTCCTTGGGGTGTGTTCTCAGCCAGGAGTGC |
| | ACCAAATTTAAAGTCTCCAGCTGCCGGGAGTGTATCGAGAGCGGCCCAGGCTGCA |
| | CATGGTGCCAAAAACTCAACTTCACCGGACCAGGAGATCCCGACAGTATAAGATG |
| | TGATACTAGGCCTCAGCTGTTGATGCGGGGCTGTGCTGCCGACGACATTATGGAC |
| | CCTACCAGCTTAGCAGAAACCCAGGAAGATCATAATGGGGGGCAAAAACAGCTCA |
| | GCCCCCAGAAAGTGACCCTGTACCTGCGACCCGGTCAAGCGGCTGCCTTCAACGT |
| | GACTTTTCGGCGCGCGAAGGGATACCCCATCGATCTTTATTATCTGATGGATTTG |
| | AGCTACTCAATGCTGGATGACCTGAGGAACGTAAAGAAGTTGGGAGGGGATCTGC |
| | TGCGCGCTCTTAATGAAATTACAGAATCAGGCAGGATCGGCTTCGGATCATTCGT |
| | GGACAAAACAGTGCTGCCGTTCGTGAACACACACCCGGACAAGCTAAGAAATCCT |
| | TGCCCCAACAAGGAAAAGGAGTGCCAGCCGCCATTTGCTTTCCGACACGTGCTGA |
| | AACTCACGAATAACTCAAATCAATTCCAAACCGAAGTGGGCAAGCAGCTGATCTC |
| | TGGCAACCTGGACGCCCCCGAGGGGGGACTAGATGCGATGATGCAAGTTGCCGCT |
| | TGCCCCGAAGAGATTGGCTGGCGCAACGTGACCAGACTGCTGGTCTTTGCAACTG |
| | ATGACGGCTTCCATTTTGCTGGCGATGGAAAGTTGGGGGCAATCCTGACACCTAA |
| | TGACGGCCGCTGCCATCTAGAAGATAACCTCTATAAGAGGAGTAATGAGTTTGAC |
| | TATCCAAGTGTCGGCCAGCTGGCACATAAACTAGCCGAGAACAACATCCAACCGA |
| | TCTTCGCAGTCACAAGTAGGATGGTAAAGACGTACGAGAAGCTGACTGAGATCAT |
| | ACCAAAATCTGCAGTGGGGGAGCTGAGCGAAGATTCTAGCAATGTTGTGCAGCTT |
| | ATTAAGAATGCCTATAATAAGCTTTCCAGTAGAGTGTTTCTGGACCACAATGCCC |
| | TTCCCGACACTCTGAAAGTTACTTACGATTCTTTTTGTTCCAACGGCGTGACGCA |
| | CCGGAATCAGCCTCGCGGTGACTGTGACGGCGTGCAGATCAACGTGCCTATTACA |
| | TTTCAGGTTAAAGTCACTGCCACCGAGTGCATCCAGGAGCAATCATTTGTCATCA |
| | GGGCTCTCGGTTTCACGGACATTGTGACCGTTCAGGTGCTCCCTCAGTGCGAATG |
| | TCGATGCAGAGATCAGAGCAGAGATAGGTCCTTATGTCACGGGAAGGGTTTCTTG |
| | GAATGTGGGATATGCAGATGCGATACAGGCTATATAGGGAAGAACTGCGAATGCC |
| | AGACACAGGGCCGATCGTCCCAGGAGCTGGAGGGGAGCTGTCGCAAAGATAACAA |
| | CTCTATAATTTGCAGCGGGCTCGGTGACTGTGTGTGTGGTCAGTGTCTCTGCCAC |
| | ACTAGTGACGTACCAGGGAAACTCATTTATGGCCAGTACTGCGAGTGTGATACCA |
| | TTAATTGTGAGAGGTATAACGGACAGGTCTGCGGCGGGCCAGGCCGTGGACTTTG |
| | TTTCTGCGGTAAGTGCCGATGCCATCCTGGATTTGAAGGCAGTGCTTGTCAATGC |
| | GAGCGTACCACTGAGGGCTGTTTAAACCCTCGGCGTGTCGAGTGCTCGGGACGCG |
| | GTAGATGCCGGTGTAACGTCTGCGAGTGCCACTCTGGTTACCAGCTCCCCCTCTG |
| | TCAGGAATGTCCCGGATGCCCTTCTCCTTGCGGGAAGTATATTTCCTGTGCAGAA |
| | TGTCTGAAGTTTGAGAAGGGCCCCTTCGGGAAGAACTGCTCCGCCGCGTGCCCAG |
| | GACTTCAGTTATCAAATAATCCAGTAAAAGGGAGAACCTGTAAGGAAAGGGATTC |
| | CGAAGGCTGTTGGGTGGCTTACACACTGGAGCAGCAAGACGGTATGGACAGATAC |
| | CTTATCTACGTCGACGAGTCTCGGGAGTGTGTGGCCGGACCAAATATCGCTGCCA |
| | TCGTCGGCGGAACAGTGGCCGGAATTGTTCTGATCGGGATACTTCTACTCGTTAT |
| | CTGGAAAGCATTGATTCACTTGTCTGACCTGCGTGAATACCGGCGGTTCGAGAAA |
| | GAAAAGTTGAAATCGCAGTGGAATAATGACAATCCCCTCTTTAAATCAGCCACCA |
| | CTACCGTGATGAACCCCAAGTTCGCCGAGTCCTAAAACGCCATgaattcactcct |
| | caggtgcaggctgcctatcagaaggtggtggctggtgtggccaatgccctggctc |
| | acaaataccactgagatctttttccctctgccaaaaattatggggacatcatgaa |
| | gccccttgagcatctgacttctggctaataaaggaaatttattttcattgcaata |
| | gtgtgttggaattttttgtgtctctcactcggaaggacatatgggagggcaaatc |
| | atttaaaacatcagaatgagtatttggtttagagtttggcaacatatgcccatat |
| | gctggctgccatgaacaaaggttggctataaagaggtcatcagtatatgaaacag |
| | ccccctgctgtccattccttattccatagaaaagccttgacttgaggttagattt |
| | tttttatattttgttttgtgttatttttttctttaacatccctaaaattttcctt |
| | acatgttttactagccagatttttcctcctctcctgactactcccagtcatagct |
| | gtccctcttctcttatggagatCAGTTCGCTCGCtcttttgcaatgctctttggg |
| | aaattatattagcctaattactaatttcctggccctcaaggtgacttgcttgaac |
| | ttgccacctTCGCCACTAGGGGGCAGCAttggtttacacagggtgaacaaacgtt |
| | caattctaaactaatattctttggtgggaaagtgtgttcattttggtttgttttg |
| | aagaagtcacttcatacctttgaactggggttaagctggttaactcccaaaattc |
| | caccagttccaatatcctatggaaaaaccccaaaaccactCAAAggatctgcgat |
| | cgctccggtgcccgtcagtgggcagagcgcacatcgcccacagtccccgagaagt |
| | tggggggaggggtcggcaattgaacgggtgcctagagaaggtggcgcggggtaaa |
| | ctgggaaagtgatgtcgtgtactggctccgcctttttcccgagggtgggggagaa |
| | ccgtatataagtgcagtagtcgccgtgaacgttctttttcgcaacgggtttgccg |
| | ccagaacacagctgaagcttcgaggggctcgcatctctccttcacgcgcccgccg |
| | ccctacctgaggccgccatccacgccggttgagtcgcgttctgccgcctcccgcc |
| | tgtggtgcctcctgaactgcgtccgccgtctaggtaagtttaaagctcaggtcga |
| | gTccgggcctttgtccggcgctcccttggagcctacctagactcagccggctctc |
| | cacgctttgcctgaccctgcttgctcaactctacgtctttgtttcgttttctgtt |
| | ctgcgccgttacagatccaagctgtgaccggcgcctacGAATGCCGCCACCATGC |
| | TGTCAACATCCCGGTCTAGATTCATAAGAAACACCAATGAGAGTGGCGAAGAGGT |
| | GACCACTTTCTTCGATTATGATTATGGAGCCCCCTGTCATAAATTTGACGTCAAA |
| | CAGATCGGTGCACAGCTGCTGCCACCTCTTTATAGCCTCGTGTTTATTTTCGGGT |
| | TCGTGGGGAATATGCTCGTGGTTTTGATCCTCATCAACTGCAAGAAGCTCAAATG |
| | TCTGACCGATATATACCTACTGAACCTGGCCATTTCAGATCTGCTCTTCCTGATT |
| | ACGCTGCCACTCTGGGCACATTCGGCCGCTAACGAATGGGTGTTTGGTAACGCTA |
| | TGTGCAAGCTGTTCACAGGTCTTTATCATATTGGATACTTCGGCGGGATTTTCTT |
| | TATCATCCTGTTGACTATTGACCGATACCTTGCTATCGTACACGCAGTTTTCGCC |
| | CTTAAGGCGAGGACAGTCACCTTTGGTGTAGTGACATCCGTCATCACTTGGTTAG |
| | TGGCCGTTTTCGCTAGCGTGCCCGGGATTATCTTTACTAAATGCCAGAAAGAGGA |
| | TTCCGTGTATGTATGTGGGCCCTACTTCCCGAGGGGCTGGAATAACTTTCACACA |
| | ATTATGCGAAATATCCTAGGCCTTGTCTTGCCTCTTCTGATCATGGTGATATGTT |
| | ATTCTGGAATCCTGAAAACATTACTCAGATGCCGCAATGAAAAGAAGAGGCACCG |
| | GGCCGTGCGCGTCATTTTTACCATCATGATTGTGTACTTCCTATTTTGGACGCCG |
| | TACAATATCGTGATTTTGTTGAACACCTTCCAGGAGTTTTTTGGACTGTCTAACT |
| | GCGAAAGCACTTCACAGTTAGACCAGGCGACTCAAGTGACGGAGACACTGGGCAT |
| | GACCCACTGCTGTATAAATCCCATAATATACGCCTTCGTAGGCGAGAAGTTTCGT |
| | AGTCTGTTTCACATTGCCTTGGGGTGTCGCATCGCACCTCTCCAAAAGCCAGTTT |
| | GCGGGGGACCAGGCGTCAGACCTGGCAAAAATGTTAAGGTCACCACCCAGGGCCT |
| | GCTCGACGGACGGGGGAAGGGTAAAAGCATCGGAAGGGCACCCGAAGCTTCCCTG |
| | CAAGACAAGGAGGGCGCTTAGAACGCCATgggggcatccctgtgacccctcccca |
| | gtgcctctcctggccctggaagttgccactccagtgcccaccagccttgtcctaa |
| | taaaattaagttgcatcattttgtctgactaggtgtccttctataatattatggg |
| | gtggaggggggtggtatggagcaaggggcaagttgggaagagaacctgtagggcc |
| | tgcggggtctattgggaaccaagctggagtgcagtggcacaatcttggctcactg |
| | caatctccgcctcctgggttcaagcgattctcctgcctcagcctcccgagttgtt |
| | gggattccaggcatgcatgaccaggctcagctaatttttgtttttttggtagagT |
| | cggggtttcaccatattggccaggctggtgtccaactcctaatctcaggtgatct |
| | acccaccttggcctcccaaattgctgggattacaggcgtgaaccactgctccctt |
| | ccctgtccttctgattttaaaataactataccagcaggaggacgtccagacacag |
| | cataggctacctggccatgcccaaccggtgggacatttgagttgcttgcttggca |
| | ctgtcctctcatgcgttgggtccactcagtagatgcctgttCAGTCAGTTCGCtc |
| | ttttgcaatgctctttgggaaattatattagcctaattactaatttcctggccct |
| | caaggtgacttgcttgaacttgccacctTCGCCACTAGGGGGCAGCAttggttta |
| | cacagggtgaacaaacgttcaattctaaactaatattctttggtgggaaagtgtg |
| | ttcattttggtttgttttgaagaagtcacttcatacctttgaactggggttaagc |
| | tggttaactcccaaaattccaccagttccaatatcctatggaaaaaccccaaaac |
| | cactCAAAggatctgcCatcgctccggtgcccgtcagtgggcagagcgcacatcg |
| | cccacagtccccgagaagttggggggaggggtcggcaattgaacgggtgcctaga |
| | gaaggtggcgcggggtaaactgggaaagtgatgtcgtgtactggctccgcctttt |
| | tcccgagggtgggggagaaccgtatataagtgcagtagtcgccgtgaacgttctt |
| | tttcgcaacgggtttgccgccagaacacagctgaagcttcgaggggctcgcatct |
| | ctccttcacgcgcccgccgccctacctgaggccgccatccacgccggttgagtcg |
| | cgttctgccgcctcccgcctgtggtgcctcctgaactgcgtccgccgtctaggta |
| | agtttaaagctcaggtcgagTccgggcctttgtccggcgctcccttggagcctac |
| | ctagactcagccggctctccacgctttgcctgaccctgcttgctcaactctacgt |
| | ctttgtttcgttttctgttctgcgccgttacagatccaagctgtgaccggcgcct |
| | acGAATGCCGCCACCATGGCTgctgaaggTtccgtcgccaggcagcctgacctct |
| | tgacctgcgacgatgagccgatccatatccccggtgccatccaaccgcatggact |
| | gctgctcgccctcgccgccgacatgacgatcgttgccggcagcgacaaccttccc |
| | gaactcaccggactggcgatcggcgccctgatcggccgctctgcggccgatgtGt |
| | tcgactcggaAacgcacaaccgtctgacgatcgccttggccgagcccggggcggc |
| | cgtcggagcaccgatcactgtcggcttcacgatgcgaaaggacgcaggcttcatc |
| | ggctcctggcatcgccatgatcagctcatcttcctGgagctGgagcctccccagc |
| | gggacgtcgccgagccgcaggcgttcttccgccgcaccaacagcgccatccgccg |
| | cctgcaggccgccgaaaccttggaaagcgcctgcgccgccgcggcgcaagaggtg |
| | cggaagattaccggcttcgatcgggtgatgatctatcgcttcgcctccgacttca |
| | gcggcgaagtgatcgcagaggatcggtgcgccgaggtcgagtcaaaactaggcct |
| | gcactatcctgcctcaaccgtgccggcgcaggcccgtcggctctataccatcaac |
| | ccggtacggatcattcccgatatcaattatcggccggtgccggtcaccccagacc |
| | tcaatccggtcaccgggcggccgattgatcttagcttcgccatcctgcgcagcgt |
| | GtcgcccgtccatctggaGttcatgcgcaacataggcatgcacggcacgatgtcg |
| | atctcgattttgcgcggcgagcgactgtggggattgatcgtttgccatcaccgaa |
| | cgccgtactacgtcgatctcgatggccgccaagcctgcgagctagtcgcccaggt |
| | tctggcctggcagatcggcgtgatggaagagTAAAACGBBABBABBABBABBABB |
| | ACCATctgtgccttctagttgccagccatctgttgtttgcccctcccccgtgcct |
| | tccttgaccctggaaggtgccactcccactgtcctttcctaataaaatgaggaaa |
| | ttgcatcgcattgtctgagtaggtgtcattctattctggggggtggggggggcag |
| | gacagcaagggggaggattgggaaggcaatagcaggcatgctggggatgcggtgg |
| | gctctatggCAGTTAGGGCGGCCGCGCCTGGGCTTACttaattaacgagagcata |
| | atattgatatgtgccaaagttgtttctgactgactaataagtataatttgtttct |
| | attatgtataggttaagctaattacttattttataatacaacatgactgttttta |
| | aagtacaaaataagtttatttttgtaaaagagagaatgtttaaaagttttgttac |
| | tttatagaagaaattttgagtttttgtttttttttaataaataaataaacataaa |
| | taaattgtttgttgaatttattattagtatgtaagtgtaaatataataaaactta |
| | atatctattcaaattaataaataaacctcgatatacagaccgataaaacacatgc |
| | gtcaattttacgcatgattatctttaacgtacgtcacaatatgattatctttcta |
| | gggttaaataatagtttctaatttttttattattcagcctgctgtcgtgaatacc |
| | gagctccaattcgccctatagtgagtcgtattacaattcactggccgtcgtttta |
| | caacgtcgtgactgggaaaaccctggcgttacccaacttaatcgccttgcagcac |
| | atccccctttcgccagctggcgtaatagcgaagaggcccgcaccgatcgcccttc |
| | ccaacagttgcgcagcctgaatggcgaatgggacgcgccctgtagcggcgcatta |
| | agcgcggcgggtgtggtggttacgcgcagcgtgaccgctacacttgccagcgccc |
| | tagcgcccgctcctttcgctttcttcccttcctttctcgccacgttcgccggctt |
| | tccccgtcaagctctaaatcgggggctccctttagggttccgatttagtgcttta |
| | cggcacctcgaccccaaaaaacttgattagggtgatggttcacgtagtgggccat |
| | cgccctgatagacggtttttcgccctttgacgttggagtccacgttctttaatag |
| | tggactcttgttccaaactggaacaacactcaaccctatctcggtctattctttt |
| | gatttataagggattttgccgatttcggcctattggttaaaaaatgagctgattt |
| | aacaaaaatttaacgcgaattttaacaaaatattaacgcttacaatttaggtggc |
| | acttttcggggaaatgtgcgcggaacccctatttgtttatttttctaaatacatt |
| | caaatatgtatccgctcatgagacaataaccctgataaatgcttcaataatattg |
| | aaaaaggaagagtatgagtattcaacatttccgtgtcgcccttattccctttttt |
| | gcggcattttgccttcctgtttttgctcacccagaaacgctggtgaaagtaaaag |
| | atgctgaagatcagttgggtgcacgagtgggttacatcgaactggatctcaacag |
| | cggtaagatccttgagagttttcgccccgaagaacgttttccaatgatgagcact |
| | tttaaagttctgctatgtggcgcggtattatcccgtattgacgccgggcaagagc |
| | aactcggtcgccgcatacactattctcagaatgacttggttgagtactcaccagt |
| | cacagaaaagcatcttacggatggcatgacagtaagagaattatgcagtgctgcc |
| | ataaccatgagtgataacactgcggccaacttacttctgacaacgatcggaggac |
| | cgaaggagctaaccgcttttttgcacaacatgggggatcatgtaactcgccttga |
| | tcgttgggaaccggagctgaatgaagccataccaaacgacgagcgtgacaccacg |
| | atgcctgtagcaatggcaacaacgttgcgcaaactattaactggcgaactactta |
| | ctctagcttcccggcaacaattaatagactggatggaggcggataaagttgcagg |
| | accacttctgcgctcggcccttccggctggctggtttattgctgataaatctgga |
| | gccggtgagcgtgggtctcgcggtatcattgcagcactggggccagatggtaagc |
| | cctcccgtatcgtagttatctacacgacggggagtcaggcaactatggatgaacg |
| | aaatagacagatcgctgagataggtgcctcactgattaagcattggtaactgtca |
| | gaccaagtttactcatatatactttagattgatttaaaacttcatttttaattta |
| | aaaggatctaggtgaagatcctttttgataatctcatgaccaaaatcccttaacg |
| | tgagttttcgttccactgagcgtcagaccccgtagaaaagatcaaaggatcttct |
| | tgagatcctttttttctgcgcgtaatctgctgcttgcaaacaaaaaaaccaccgc |
| | taccagcggtggtttgtttgccggatcaagagctaccaactctttttccgaaggt |
| | aactggcttcagcagagcgcagataccaaatactgttcttctagtgtagccgtag |
| | ttaggccaccacttcaagaactctgtagcaccgcctacatacctcgctctgctaa |
| | tcctgttaccagtggctgctgccagtggcgataagtcgtgtcttaccgggttgga |
| | ctcaagacgatagttaccggataaggcgcagcggtcgggctgaacggggggttcg |
| | tgcacacagcccagcttggagcgaacgacctacaccgaactgagatacctacagc |
| | gtgagctatgagaaagcgccacgcttcccgaagggagaaaggcggacaggtatcc |
| | ggtaagcggcagggtcggaacaggagagcgcacgagggagcttccagggggaaac |
| | gcctggtatctttatagtcctgtcgggtttcgccacctctgacttgagcgtcgat |
| | ttttgtgatgctcgtcaggggggcggagcctatggaaaaacgccagcaacgcggc |
| | ctttttacggttcctggccttttgctggccttttgctcacatgttctttcctgcg |
| | ttatcccctgattctgtggataaccgtattaccgcctttgagtgagctgataccg |
| | ctcgccgcagccgaacgaccgagcgcagcgagtcagtgagcgaggaagcggaaga |
| | gcgcccaatacgcaaaccgcctctccccgcgcgttggccgattcattaatgcagc |
| | tggcacgacaggtttcccgactggaaagcgggcagtgagcgcaacgcaattaatg |
| | tgagttagctcactcattaggcaccccaggctttacactttatgcttccggctcg |
| | tatgttgtgtggaattgtgagcggataacaatttcacacaggaaacagctatgac |
| | catgattacgccaagctcgaaattaaccctcactaaagggaacaaaagctggtac |
| | ctcgcgcgacttggtttgccattctttagcgcgcgtcgcgtcacacagcttggcc |
| | acaatgtggtttttgtcaaacgaagattctatgacgtgtttaaagtttaggtcga |
| | gtaaagcgcaaatc |
| | (SEQ ID NO: 43) |
| pAJW_MsPBkB- | gcgcaaatcttttttaaccctagaaagatagtctgcgtaaaattgacgcatgcat |
| Blasticidin | tcttgaaatattgctctctctttctaaatagcgcgaatccgtcgctgtgcattta |
| | ggacatctcagtcgccgcttggagctcccgtgaggcgtgcttgtcaatgcggtaa |
| | gtgtcactgattttgaactataacgaccgcgtgagtcaaaatgacgcatgattat |
| | cttttacgtgacttttaagatttaactcatacgataattatattgttatttcatg |
| | ttctacttacgtgataacttattatatatatattttcttgttatagatatcatca |
| | actttgtatagaaaagttgctcgacattgattattgactagttattaatagtaat |
| | caattacggggtcattagttcatagcccatatatggagttccgcgttacataact |
| | tacggtaaatggcccgcctggctgaccgcccaacgacccccgcccattgacgtca |
| | ataatgacgtatgttcccatagtaacgccaatagggactttccattgacgtcaat |
| | gggtggagtatttacggtaaactgcccacttggcagtacatcaagtgtatcatat |
| | gccaagtacgccccctattgacgtcaatgacggtaaatggcccgcctggcattat |
| | gcccagtacatgaccttatgggactttcctacttggcagtacatctacgtattag |
| | tcatcgctattaccatggtcgaggtgagccccacgttctgcttcactctccccat |
| | ctcccccccctccccacccccaattttgtatttatttattttttaattattttgt |
| | gcagcgatgggggcggggggggggggggggcgcgcgccaggcggggggggcgggg |
| | cgaggggggggcggggcgaggcggagaggtgcggcggcagccaatcagagcggcg |
| | cgctccgaaagtttccttttatggcgaggcggcggcggcggcggccctataaaaa |
| | gcgaagcgcgcggcggggggagtcgctgcgcgctgccttcgccccgtgccccgct |
| | ccgccgccgcctcgcgccgcccgccccggctctgactgaccgcgttactcccaca |
| | ggtgagcgggcgggacggcccttctcctccgggctgtaattagcgcttggtttaa |
| | tgacggcttgtttcttttctgtggctgcgtgaaagccttgaggggctccgggagg |
| | gccctttgtgcggggggagcggctcggggggtgcgtgcgtgtgtgtgtgcgtggg |
| | gagcgccgcgtgcggctccgcgctgcccggcggctgtgagcgctgcgggcgcggc |
| | gcggggctttgtgcgctccgcagtgtgcgcgaggggagcgcggccgggggcggtg |
| | ccccgcggtgcggggggggctgcgaggggaacaaaggctgcgtgcggggtgtgtg |
| | cgtgggggggtgagcagggggtgtgggcgcgtcggtcgggctgcaaccccccctg |
| | cacccccctccccgagttgctgagcacggcccggcttcgggtgcggggctccgta |
| | cggggcgtggcgcggggctcgccgtgccgggcggggggggcggcaggtgggggtg |
| | ccgggcggggggggccgcctcgggccggggagggctcgggggaggggcgcggcgg |
| | cccccggagcgccggcggctgtcgaggcgcggcgagccgcagccattgcctttta |
| | tggtaatcgtgcgagagggcgcagggacttcctttgtcccaaatctgtgcggagc |
| | cgaaatctgggaggcgccgccgcaccccctctagcgggcgcggggcgaagcggtg |
| | cggcgccggcaggaaggaaatgggcggggagggccttcgtgcgtcgccgcgccgc |
| | cgtccccttctccctctccagcctcggggctgtccgcggggggacggctgccttc |
| | gggggggacggggcagggcggggttcggcttctggcgtgtgaccggcggctctag |
| | agcctctgctaaccatgttcatgccttcttctttttcctacagctcctgggcaac |
| | gtgctggttattgtgctgtctcatcattttggcaaagaattgcaagtttgtacaa |
| | aaaagcaggctGAATGCCGCCACCatggccaagcctttgtctcaagaagaatcca |
| | ccctcattgaaagagcaacggctacaatcaacagcatccccatctctgaggacta |
| | cagcgtcgccagcgcagctctctctagcgacggccgcatcttcactggtgtcaat |
| | gtatatcattttactgggggaccttgtgcagaactcgtggtgctgggcactgctg |
| | ctgctgcggcagctggcaacctgacttgtatcgtcgcgatcggaaatgagaacag |
| | gggcatcttgagcccctgcggacggtgccgacaggtgcttctcgatctgcatcct |
| | gggatcaaagccatagtgaaggacagtgatggacagccgacggcagttgggattc |
| | gtgaattgctgccctctggttatgtgtgggagggctaaAACGCCATgatccagac |
| | atgataagatacattgatgagtttggacaaaccacaactagaatgcagtgaGGCG |
| | CGCCTGGATACTTCTGCCTGCAGGGGAGATGTCTTCCAAAttgacggctagctca |
| | gtcctaggtacagtgctagcaTACCAGATAAGTTCGTCGGTCGGTTATGCAGTTT |
| | AAGGTTTACACCTATAAAAGAGAGAGCCGTTATCGTCTGTTTGTGGATGTACAGA |
| | GTGATATTATTGACACGCCCGGGCGACGGATGGTGATCCCCCTGGCCAGTGCACG |
| | TCTGCTGTCAGATAAAGTCTCCCGTGAACTTTACCCGGTGGTGCATATCGGGGAT |
| | GAAAGCTGGCGCATGATGACCACCGATATGGCCAGTGTGCCGGTGTCCGTTATCG |
| | GGGAAGAAGTGGCTGATCTCAGCCACCGCGAAAATGACATCAAAAACGCCATTAA |
| | CCTGATGTTCTGGGGAATATAAtaaTGGCctcggtaccaaattccagaaaagagg |
| | cctcccgaaaggggggccttttttcgttttggtccgTGGCggaccaaaacgaaaa |
| | aagacgcttttcagcgtcttattgttcgtctttggtaccgagGCCATTAtttgta |
| | tagttcatccatgccaccggtagaatgacgaccctccgcgcgctcatattgctct |
| | acgatcgtataatcttcattatgagataagatgtccagtttaatatccacattgt |
| | acgcgccaggcagctgcacaggtttcttggctttgtacgtggttttcacttcagc |
| | gtcataatggccgccatctttcagtttcaggcgctgtttaatttcgcctttcagc |
| | gcaccatcttccggatacatgcgttcggtagacgcctcccaacccatcgtcctac |
| | actgcatcaccgggccatcagatggaaaattagtaccacgcagtttaactttata |
| | gatgaactcgccatcctgAagggaggagtcctgagtgacggtcacgacaccacca |
| | tcttcaaaattcattacgcgttcccagttgaaaccttccggaaaagacagcttca |
| | gatagtccgggatatccgctgggtgtttaacatacgctttagaaccgtacataaa |
| | ttgcgggctcaggatgtcccacgcaaaaggcagcgggccgcctttagtcactttc |
| | agtttggcggtctgggtgccttcatacggacggccctcgccttcgccttcgatct |
| | cgaactcgtgaccgttaacagaaccctccatgtgaactttgaagcgcatgaactc |
| | tttaatgatagctaagttgttttcttctcctttactcacCATTtagtaTTTCTCC |
| | TCTTTcactagGTCGagtagctagcataatacctaggactgagctagctgtaaaG |
| | AAGGAAGACATCAGTGCGGCCGCGCCTGGGCTTACttaattaacgagagcataat |
| | attgatatgtgccaaagttgtttctgactgactaataagtataatttgtttctat |
| | tatgtataggttaagctaattacttattttataatacaacatgactgtttttaaa |
| | gtacaaaataagtttatttttgtaaaagagagaatgtttaaaagttttgttactt |
| | tatagaagaaattttgagtttttgtttttttttaataaataaataaacataaata |
| | aattgtttgttgaatttattattagtatgtaagtgtaaatataataaaacttaat |
| | atctattcaaattaataaataaacctcgatatacagaccgataaaacacatgcgt |
| | caattttacgcatgattatctttaacgtacgtcacaatatgattatctttctagg |
| | gttaaataatagtttctaatttttttattattcagcctgctgtcgtgaataccga |
| | gctccaattcgccctatagtgagtcgtattacaattcactggccgtcgttttaca |
| | acgtcgtgactgggaaaaccctggcgttacccaacttaatcgccttgcagcacat |
| | ccccctttcgccagctggcgtaatagcgaagaggcccgcaccgatcgcccttccc |
| | aacagttgcgcagcctgaatggcgaatgggacgcgccctgtagcggcgcattaag |
| | cgcggcgggtgtggtggttacgcgcagcgtgaccgctacacttgccagcgcccta |
| | gcgcccgctcctttcgctttcttcccttcctttctcgccacgttcgccggctttc |
| | cccgtcaagctctaaatcgggggctccctttagggttccgatttagtgctttacg |
| | gcacctcgaccccaaaaaacttgattagggtgatggttcacgtagtgggccatcg |
| | ccctgatagacggtttttcgccctttgacgttggagtccacgttctttaatagtg |
| | gactcttgttccaaactggaacaacactcaaccctatctcggtctattcttttga |
| | tttataagggattttgccgatttcggcctattggttaaaaaatgagctgatttaa |
| | caaaaatttaacgcgaattttaacaaaatattaacgcttacaatttaggtggcac |
| | ttttcggggaaatgtgcgcggaacccctatttgtttatttttctaaatacattca |
| | aatatgtatccgctcatgagacaataaccctgataaatgcttcaataatattgaa |
| | aaaggaagagtatgagtattcaacatttccgtgtcgcccttattcccttttttgc |
| | ggcattttgccttcctgtttttgctcacccagaaacgctggtgaaagtaaaagat |
| | gctgaagatcagttgggtgcacgagtgggttacatcgaactggatctcaacagcg |
| | gtaagatccttgagagttttcgccccgaagaacgttttccaatgatgagcacttt |
| | taaagttctgctatgtggcgcggtattatcccgtattgacgccgggcaagagcaa |
| | ctcggtcgccgcatacactattctcagaatgacttggttgagtactcaccagtca |
| | cagaaaagcatcttacggatggcatgacagtaagagaattatgcagtgctgccat |
| | aaccatgagtgataacactgcggccaacttacttctgacaacgatcggaggaccg |
| | aaggagctaaccgcttttttgcacaacatgggggatcatgtaactcgccttgatc |
| | gttgggaaccggagctgaatgaagccataccaaacgacgagcgtgacaccacgat |
| | gcctgtagcaatggcaacaacgttgcgcaaactattaactggcgaactacttact |
| | ctagcttcccggcaacaattaatagactggatggaggcggataaagttgcaggac |
| | cacttctgcgctcggcccttccggctggctggtttattgctgataaatctggagc |
| | cggtgagcgtgggtctcgcggtatcattgcagcactggggccagatggtaagccc |
| | tcccgtatcgtagttatctacacgacggggagtcaggcaactatggatgaacgaa |
| | atagacagatcgctgagataggtgcctcactgattaagcattggtaactgtcaga |
| | ccaagtttactcatatatactttagattgatttaaaacttcatttttaatttaaa |
| | aggatctaggtgaagatcctttttgataatctcatgaccaaaatcccttaacgtg |
| | agttttcgttccactgagcgtcagaccccgtagaaaagatcaaaggatcttcttg |
| | agatcctttttttctgcgcgtaatctgctgcttgcaaacaaaaaaaccaccgcta |
| | ccagcggtggtttgtttgccggatcaagagctaccaactctttttccgaaggtaa |
| | ctggcttcagcagagcgcagataccaaatactgttcttctagtgtagccgtagtt |
| | aggccaccacttcaagaactctgtagcaccgcctacatacctcgctctgctaatc |
| | ctgttaccagtggctgctgccagtggcgataagtcgtgtcttaccgggttggact |
| | caagacgatagttaccggataaggcgcagcggtcgggctgaacggggggttcgtg |
| | cacacagcccagcttggagcgaacgacctacaccgaactgagatacctacagcgt |
| | gagctatgagaaagcgccacgcttcccgaagggagaaaggcggacaggtatccgg |
| | taagcggcagggtcggaacaggagagcgcacgagggagcttccagggggaaacgc |
| | ctggtatctttatagtcctgtcgggtttcgccacctctgacttgagcgtcgattt |
| | ttgtgatgctcgtcaggggggcggagcctatggaaaaacgccagcaacgcggcct |
| | ttttacggttcctggccttttgctggccttttgctcacatgttctttcctgcgtt |
| | atcccctgattctgtggataaccgtattaccgcctttgagtgagctgataccgct |
| | cgccgcagccgaacgaccgagcgcagcgagtcagtgagcgaggaagcggaagagc |
| | gcccaatacgcaaaccgcctctccccgcgcgttggccgattcattaatgcagctg |
| | gcacgacaggtttcccgactggaaagcgggcagtgagcgcaacgcaattaatgtg |
| | agttagctcactcattaggcaccccaggctttacactttatgcttccggctcgta |
| | tgttgtgtggaattgtgagcggataacaatttcacacaggaaacagctatgacca |
| | tgattacgccaagctcgaaattaaccctcactaaagggaacaaaagctggtacct |
| | cgcgcgacttggtttgccattctttagcgcgcgtcgcgtcacacagcttggccac |
| | aatgtggtttttgtcaaacgaagattctatgacgtgtttaaagtttaggtcgagt |
| | aaa |
| | (SEQ ID NO: 44) |
| TSG-6 CDS | ATGATCATTCTTATTTATCTGTTTCTGCTGTTGTGGGAGGATACTCAAGGATGGG |
| | GTTTCAAGGATGGCATCTTCCACAACTCCATCTGGCTGGAGCGCGCTGCCGGGGT |
| | TTACCACAGAGAGGCTAGGTCTGGGAAGTACAAACTCACTTATGCCGAAGCTAAG |
| | GCAGTGTGTGAATTCGAAGGTGGTCATTTGGCCACATACAAGCAGCTGGAGGCGG |
| | CCAGGAAAATTGGATTCCACGTCTGCGCAGCCGGGTGGATGGCTAAAGGCCGCGT |
| | GGGCTATCCGATAGTCAAGCCCGGACCCAACTGCGGGTTCGGAAAGACAGGCATC |
| | ATTGATTACGGCATTCGTCTGAATAGATCAGAAAGATGGGACGCATACTGTTACA |
| | ATCCTCATGCAAAAGAGTGCGGAGGAGTGTTTACCGACCCCAAGCAAATATTTAA |
| | AAGCCCTGGTTTCCCAAACGAGTATGAGGATAATCAGATATGTTACTGGCATATC |
| | CGACTCAAGTACGGACAGCGGATTCACCTCAGCTTCCTGGATTTCGATCTTGAAG |
| | ATGACCCAGGGTGTCTCGCCGACTACGTAGAGATCTACGACAGTTATGACGACGT |
| | GCATGGTTTCGTGGGCCGGTATTGCGGCGACGAATTGCCAGATGATATTATCAGC |
| | ACAGGGAATGTGATGACGCTTAAGTTTCTATCTGACGCTAGCGTCACCGCGGGGG |
| | GGTTTCAGATCAAGTATGTAGCCATGGACCCTGTTTCCAAAAGTTCACAGGGCAA |
| | AAACACATCGACCACTTCCACCGGCAATAAAAACTTTCTGGCCGGCAGGTTTTCT |
| | CACTTA |
| | (SEQ ID NO: 45) |
| COX2 CDS | ATGGCTCACGCTGCACAGGTGGGGCTGCAGGATGCAACTAGCCCTATCATGGAGG |
| | AGCTGATTACATTTCATGACCATGCACTGATGATAATTTTCTTAATCTGTTTTCT |
| | TGTGCTGTATGCCCTATTCTTGACTCTGACGACCAAGTTGACAAACACCAATATC |
| | AGCGACGCCCAGGAAATGGAGACTGTCTGGACCATACTCCCCGCCATTATTTTAG |
| | TGCTCATAGCCCTGCCATCCCTGCGCATCTTGTACATGACCGATGAAGTCAATGA |
| | CCCATCACTCACTATTAAATCTATCGGTCACCAGTGGTACTGGACATATGAGTAC |
| | ACAGATTACGGCGGTCTGATCTTTAACTCTTATATGTTACCGCCCCTCTTTCTGG |
| | AGCCGGGCGACCTTAGACTCCTGGACGTTGATAACCGTGTAGTCCTGCCCATTGA |
| | AGCCCCTATACGAATGATGATCACCAGTCAGGATGTTCTGCATTCATGGGCTGTG |
| | CCAACGCTGGGACTCAAAACTGACGCGATTCCCGGCAGGCTTAACCAAACCACCT |
| | TCACAGCTACACGGCCTGGGGTGTACTATGGGCAATGCTCCGAGATCTGCGGAGC |
| | CAATCACTCGTTCATGCCAATTGTATTGGAACTAATCCCCCTCAAGATCTTCGAA |
| | ATGGGACCTGTGTTTACCCTT |
| | (SEQ ID NO: 46) |
| IDO1 CDS | ATGGCCCATGCCATGGAGAATAGCTGGACCATCTCTAAGGAATATCACATTGACG |
| | AAGAGGTAGGATTCGCCCTTCCTAACCCTCAGGAAAATCTGCCAGACTTTTACAA |
| | CGATTGGATGTTTATCGCTAAGCACCTTCCCGATCTGATCGAATCGGGACAGCTG |
| | AGAGAAAGGGTCGAGAAACTGAATATGCTGTCAATTGACCACCTGACTGACCACA |
| | AAAGTCAGCGGCTCGCACGCTTGGTACTGGGTTGCATCACGATGGCATATGTCTG |
| | GGGTAAAGGACATGGCGATGTGAGAAAGGTTCTGCCACGGAACATAGCCGTGCCC |
| | TATTGTCAGCTGTCCAAGAAACTTGAGCTGCCCCCAATCCTGGTTTACGCGGATT |
| | GCGTGCTAGCTAACTGGAAGAAGAAAGACCCAAACAAACCCCTGACCTATGAGAA |
| | CATGGATGTGTTATTTAGCTTTCGAGATGGGGATTGTTCAAAGGGCTTCTTCCTT |
| | GTGTCCCTTCTGGTCGAGATAGCCGCCGCATCCGCCATCAAAGTGATCCCTACCG |
| | TTTTTAAAGCTATGCAGATGCAGGAACGCGACACTCTCCTCAAAGCCTTATTGGA |
| | AATCGCTTCTTGCTTGGAGAAAGCCTTACAGGTGTTTCACCAAATTCATGACCAT |
| | GTGAATCCTAAAGCGTTCTTCAGCGTGCTCAGGATTTACCTAAGCGGCTGGAAGG |
| | GGAATCCCCAGCTGTCGGATGGCCTCGTATACGAAGGCTTTTGGGAGGACCCAAA |
| | GGAGTTCGCAGGTGGATCAGCTGGGCAATCTTCTGTGTTCCAATGCTTCGATGTT |
| | CTGCTGGGGATTCAGCAGACTGCAGGTGGCGGCCATGCTGCACAGTTCCTCCAGG |
| | ACATGCGCAGGTACATGCCTCCAGCTCACAGAAATTTTCTCTGTAGTCTTGAAAG |
| | TAACCCGTCCGTTCGTGAGTTTGTCTTGAGTAAGGGCGATGCAGGACTCCGGGAG |
| | GCGTACGACGCCTGTGTCAAAGCCCTGGTGTCCTTGCGAAGCTACCACTTACAAA |
| | TAGTGACCAAGTATATTTTGATTCCCGCTTCACAGCAACCGAAGGAAAACAAGAC |
| | AAGCGAGGACCCTTCCAAACTCGAGGCCAAGGGAACGGGGGGGACAGACCTCATG |
| | AATTTCCTAAAGACAGTCAGGTCTACAACCGAGAAGAGCCTGCTGAAGGAAGGC |
| | (SEQ ID NO: 47) |
| CTLA4 CDS | ATGGCCTGCCTGGGGTTTCAGAGGCACAAGGCCCAGCTGAATCTCGCAACGCGTA |
| | CATGGCCTTGCACCCTGCTGTTCTTCCTGTTGTTTATCCCAGTTTTTTGTAAGGC |
| | CATGCATGTTGCCCAGCCAGCAGTGGTCCTCGCATCATCGAGAGGGATCGCTTCA |
| | TTTGTGTGCGAGTATGCTAGTCCAGGCAAAGCCACAGAAGTCCGAGTGACAGTAT |
| | TGAGACAGGCTGACAGTCAGGTCACTGAGGTCTGCGCTGCCACTTATATGATGGG |
| | AAATGAACTTACCTTCCTCGACGATTCTATTTGTACCGGTACCAGCAGCGGCAAC |
| | CAGGTAAACCTAACAATACAAGGCCTCCGCGCGATGGACACGGGTCTATATATTT |
| | GTAAAGTGGAGCTCATGTACCCTCCCCCTTACTACCTGGGCATTGGAAATGGCAC |
| | ACAAATCTACGTGATAGATCCGGAACCTTGTCCCGATTCTGACTTTCTTCTGTGG |
| | ATTCTTGCAGCGGTTTCCAGCGGATTATTCTTCTATAGCTTTCTGTTGACCGCCG |
| | TGTCCCTGTCTAAGATGTTAAAGAAGCGGTCCCCCCTGACCACTGGGGTGTACGT |
| | GAAAATGCCACCCACTGAACCTGAGTGCGAGAAACAGTTCCAACCCTACTTCATC |
| | CCGATCAAC |
| | (SEQ ID NO: 48) |
| HLA-G CDS | ATGGTGGTGATGGCCCCTAGGACCCTTTTCCTGCTTTTGAGTGGGGCACTGACAC |
| | TGACCGAAACGTGGGCCGGTAGTCATTCCATGCGATATTTCTCAGCAGCCGTCAG |
| | CAGACCCGGAAGAGGAGAGCCTCGCTTCATTGCCATGGGTTACGTCGATGATACA |
| | CAGTTCGTACGGTTTGATTCTGATTCCGCTTGCCCTAGGATGGAGCCGCGAGCAC |
| | CATGGGTGGAACAGGAGGGCCCTGAATATTGGGAGGAGGAGACACGGAATACCAA |
| | GGCTCACGCCCAGACTGATAGGATGAACTTACAAACCCTGAGGGGGTACTACAAT |
| | CAAAGCGAAGCTTCATCGCACACCCTGCAGTGGATGATCGGCTGCGATCTGGGGT |
| | CAGACGGGCGCCTCTTGCGGGGCTACGAGCAGTATGCATACGACGGCAAAGACTA |
| | TCTCGCTCTGAATGAGGACCTGCGAAGTTGGACAGCTGCGGATACCGCCGCCCAG |
| | ATCTCCAAGAGAAAATGTGAAGCCGCAAACGTCGCGGAGCAGAGAAGGGCTTACC |
| | TCGAGGGAACGTGTGTCGAATGGCTCCACAGATATCTAGAGAACGGCAAGGAGAT |
| | GCTCCAGCGGGCCGACCCACCAAAAACCCACGTGACCCATCATCCCGTTTTTGAC |
| | TATGAGGCCACTCTGCGTTGCTGGGCCCTTGGCTTTTACCCAGCTGAAATCATTC |
| | TAACTTGGCAACGCGACGGTGAGGATCAGACACAAGACGTGGAACTCGTTGAAAC |
| | GCGGCCGGCAGGCGACGGCACTTTTCAGAAATGGGCCGCTGTCGTGGTTCCCTCC |
| | GGAGAGGAACAACGTTACACTTGTCACGTGCAGCATGAAGGACTGCCAGAGCCCC |
| | TTATGTTACGCTGGAAGCAGTCTAGCCTGCCCACAATCCCTATAATGGGAATTGT |
| | AGCGGGGTTGGTTGTGCTGGCAGCAGTGGTAACTGGGGCCGCGGTGGCTGCCGTG |
| | TTGTGGCGCAAGAAGTCTAGCGAC |
| | (SEQ ID NO: 49) |

### DNA Preparation

Competent cells for all E. coli strains used in this study were prepared using the TSS method⁶⁷. For most experiments in this study, expression plasmids were transformed into NEB Stables (NEB C3040I) and prepared using a commercially available endotoxin free maxi-prep kit (Zymo D4202) prior to nucleofection into MSCs. In FIG. 20B, other E. coli strains were tested including OmniMax (ThermoFisher C854003), DH5a (ThermoFisher 18265017), DH10B (ThermoFisher EC0113), and NEB Turbo (NEB C2984I). Other commercial DNA preparation kits were also tested, including miniprep kits from both Qiagen (27104) and Zymo (D42010) and the Qiagen endotoxin free maxi-prep kit (12362) according to manufacturer's instructions. When DNA cargos were diversified (FIGS. 24-26), DNA was prepared by miniprep according to manufacturer instructions.

### Nucleofection

Nucleofection was performed on an Amaxa Nucleofector II with the 96-well Shuttle using the Lonza 96 well Nucleofector kit with P1 primary cell buffer (Lonza V4SP-1096). DNA was prepared prior to beginning cell handling by adding 1 μg of Level 3 transposon and 500 ng transposase or 1 μg Level 2 expression unit to a sterile 1.7 mL tube (Thermo Scientific 3451) in a final volume of 2 μL of endotoxin free elution buffer or water. To prepare MSCs for nucleofection, MSCs were thawed from frozen stocks at the passage described in MSC Handling, washed with complete αMEM, counted, then plated on tissue culture plastic at 10k cells/cm² and incubated overnight to plate recover. Cells were harvested and aliquoted into sterile 1.7 mL tubes with approximately 500k cells per tube per sample at 500×g for 5 min. After aspirating the media without disturbing the pellet, the cell pellet was resuspended in 20 μL P1 buffer, transferred to the 1.7 mL tube containing the DNA, then transferred to the nucleofection cuvette without introducing bubbles. The mixture was then electroporated using code EW104 or FF104 within 10-15 minutes of adding the P1 buffer and recovered for 10 min at 25° C. 100 μL of pre-warmed media was then added to the cuvette and the entire mixture was transferred to the destination well containing pre-warmed media then incubated.

After nucleofection, transient experiments were plated at 50k cells/cm² in a 6 well plate (Grenier 657 160) then harvested and counted at 48 hours. For selection, cells were plated at 10k cells/cm² in 2×T25 tissue culture flasks (Corning 430639). Media was changed at 24 h with complete αMEM and again at 72 h with complete αMEM supplemented with puromycin at 0.5 ng/ml (Millipore Sigma P8833). Cells were selected and expanded for 7-10 d, changing media periodically as needed to remove cell debris but at least every 2-3 d. Cells were harvested at 80-90% confluency and counted. Selection media was replaced with complete αMEM without puromycin for the remainder of the expansion after a transposase-only control run in parallel had been completely culled by the selection. Multiple nucleofection conditions, including reagent preparation, input ratios, and cell handling (FIG. 20), as well as selection and expansion conditions (FIG. 21), were tested. The Lonza 4D nucleofector was also tested to evaluate reproducibility (FIG. 34).

The area of pie charts representing overall yield was calculated according to the following formula:

$$Area = {π ( \begin{matrix}radius of circle representing \\ the starting population\end{matrix} )}^{2} \times \frac{Overall \% Yield}{100}$$

### Alternative Transfection Methods

Lipofectamine 3000 (ThermoFisher L3000001), Lipofectamine LTX (ThermoFisher A126201), and Fugene 4K (Fugene 4K-1000) reagents were used according to manufacturer protocols, with the following modifications: 200k MSCs per well were plated in a 6-well plate in complete αMEM for transfection the next day. Lipofectamine reagents were diluted in Opti-MEM (Gibco 51985-034). 2 μg of DNA was diluted with P3000 for Lipofectamine 3000, or PLUS reagent for LTX. Diluted DNA and Lipofectamine were mixed 1:1, incubated at 25° C. for 15 min, then added to the cells. Fugene transfection reagent was added to 2 μg of DNA diluted in Opti-MEM, mixed, incubated at 25° C. for 15 min, then added to the cells. Lipofectamine RNAiMAX (ThermoFisher 13778030) was used according to manufacturer protocols and as previously described³². Briefly, 100k MSCs per well were plated in a 6 well plate in complete αMEM for transfection the next day. The appropriate amount of Lipofectamine and the appropriate amount of GFP mRNA (SBI MR700A-2 or TriLink L-7601-100) were diluted in Opti-MEM in order to achieve ratios of 1:2 or 1:4 μg RNA:μL RNAiMAX. The diluted DNA and Lipofectamine were mixed 1:1, incubated at 25° C. for 15 min, then added to the cells. For all transfections, media was not changed, and cells were harvested, counted, and analyzed by flow cytometry at 48 h.

To assess the impact of dexamethasone on transfection and nucleofection⁵¹, complete αMEM supplemented with 120 nM dexamethasone (Millipore Sigma D4902-25 MG) was added to the cells 30 min before transfection or nucleofection (FIGS. 32-33). To assess the impact of CEPT on nucleofection, the CEPT cocktail was prepare as previously described⁵² by supplementing complete αMEM with 50 nM chroman 1 (Fisher 71-631-0), 5 μM emricasan (Fisher NC1623866), 1× polyamine supplement (Sigma P8483-5 mL), and 0.7 μM trans-ISRIB (Fisher 52-841-0). MSCs were either pre-conditioned by being plate recovered for 24 hours or cultured post-nucleofection in CEPT-containing αMEM (FIG. 33).

### Flow Cytometry

After eMSCs were harvested and counted, cells were spun down and resuspended in 300 μL autoMACs buffer (Milteni Biotech 130-091-221). The entire harvested population (˜500k cells) was assayed for experiments in FIGS. 20 and 21B, while 100k cells were assayed in all other instances. Data were acquired using a LSRII (BD Biosciences) and analyzed with FlowJo (BD Biosciences). The main population was gated to exclude doublets, cell debris, and dead cells using the forward and side scatter parameters. Typically, 10k single cell events were recorded. Reporter expression in single cells was assessed using area parameters for GFP (488 nm laser, 530 nm filter with 30 nm bandpass), BFP (405 nm laser, 450 nm filter with 50 nm bandpass), and iRFP (633 nm laser, 780 nm filter with 60 nm bandpass). Cells were assigned as FP+ if expression was greater than the negative cells. We then calculated overall yield by dividing the total number of cells harvested by the number of input cells. Nucleofection efficiency was determined as the percentage of single cells that were FP+ via flow cytometry. FP+ yield was determined by multiplying the overall yield by nucleofection efficiency. Pie charts were created by plotting the overall yield as a percentage of the area of the starting population, with the fraction of the pie representing the percentage of overall yield that was FP+ (FIG. 20A, FIG. 28).

MSC immunophenotyping was performed by surface labeling with a multicolor panel composed of fluorophore-conjugated CD34, CD45, CD73, CD90, and HLA-DR (APC Mouse Anti-Human CD34; clone #581, BD 555824. APC-Cy7 Anti-Human CD45; clone #HI30, Biolegend 304014. BV421 Anti-Human CD73; clone #AD2, Biolegend 344008. PE/Cy7 Anti-Human CD90; clone #5E10, Biolegend 328124. PerCP-Cy5.5 Mouse Anti-Human HLA-DR; clone #G46-6, BD 560652). 100k cells were resuspended in 100 μL autoMACs buffer. Antibodies were added according to manufacturer recommendations, typically 5 μL per antibody. Cells were incubated in the dark for 30 min at 25° C., then washed 2× in 1 mL autoMACs and resuspended in 300 μL autoMACs for flow cytometry.

### Splenocyte Activation Assay

Protocols involving the use of animals followed the National Institutes of Health Guide for the Care and Use of Laboratory Animals and were approved by the University of Texas Health Science Center Institutional Animal Care and Use Committee AWC-22-0043. Splenocytes were isolated as previously described⁶⁸ from Sprague-Dawley rats (250-300 g, Charles River 001CD) housed on a 12 h light/dark cycle with ad libitum access to food and water. Freshly obtained spleen was morselized by pushing it through a 70 μm mesh filter (FisherBrand 22-363-548) with the plunger of a sterile 5 ml syringe (BD 309646) to exclude connective tissue. The collected cells were suspended in ice cold PBS, centrifuged 400×g for 8 min, resuspended in 5 mL ice-cold ACK lysis buffer (Gibco A10492-01) by pipetting for 3 min, and left on ice for an additional 2 minutes. 45 mL of ice-cold PBS were added, then centrifuged at 400×g for 8 min. After aspirating the supernatant, cells were resuspended in 20 mL pre-warmed complete RPMI 1640 media (ThermoFisher 11875093) supplemented with 10% FBS (Atlanta Biologicals S11550), 1× GlutaMax, and PenStrep (100 U/mL penicillin, 100 μg/mL streptomycin, Gibco 15140-122), then passed through a 40 μm filter (FisherBrand 22-363-547). The splenocytes were counted and assayed for viability using a Vial cassette (Chemometec 941-0011) on a NucleoCounter NC-200 (Chemometec). Splenocytes were diluted to 2 M/mL in complete RPMI 1640 media. Cells were then activated with 1 μg/mL lipopolysaccharide (LPS) (Invivogen tlrl-3pelps) alone or in the presence of MSCs or eMSCs (20:1 splenocyte: MSC ratio, 200k splenocyte: 10k MSC) in 96-well flat bottom, tissue culture treated plates (Thermo Scientific 161093). Conditioned media was collected at 24 h after LPS treatment, frozen at −20° C., and analyzed by rTNFα ELISA kit (Biolegend 438204). In FIG. 26, the conditioned media was also analyzed using an hIL-10 ELISA kit (Biolegend 430604).

### T Cell Suppression Assay

Fresh human PBMCs were obtained from healthy, anonymous donors. PBMCs were isolated from whole blood using SepMate™ PBMC isolation tubes (StemCell 85450) and Ficoll-Paque PLUS medium (Cytivia 17144002) according to manufacturer's protocol and counted using a Vial Nucleocounter cassette. Following isolation, PBMCs were cultured in complete RPMI-1640 medium. PBMCs were diluted to 2×10⁶/mL in RPMI and activated with Dynabeads™ Human T-activator CD3/CD28 (Gibco 11131D) (1:1 PBMC: bead ratio) alone or in co-culture with WT MSCs or eMSCs (1:10 eMSC: PBMC ratio, 100k MSCs: 1M PBMCs) in 24-well plates at a final volume of 1 mL. After 48 h, PBMC proliferation was assayed using iClick™ EdU Andy Fluor™ 488 Flow Cytometry Assay Kit (APB Biosciences A007) according to the manufacturer's protocol with the following modifications. Cells were treated with 2 μM EdU for 30 min, then suspension cells were harvested, the Dynabeads were removed using a magnet, and cells were pelleted at 300×g for 5 min in 1.7 mL tubes. Supernatant was collected, frozen at −20° C., and analyzed using a hIFNγ ELISA kit (Biolegend 430104). Cells were transferred to a 96-well plate, washed with 200 μL PBS with 1% BSA, and fixed with 100 μL iClick fixative, washed, and then permeabilized by using 100 μL 1×iClick permeabilization and wash reagent. EdU detection was accomplished by click chemistry using a reaction scaled down 40% of the manufacturer's protocol, adding 200 μL Click-iT reaction cocktail to cells resuspended in 40 μL 1×iClick permeabilization and wash buffer. To analyze the proliferation rate of different subsets of T cells, PBMCs were stained with Ghost Dye™ Violet 450 (Cytek 13-0863-T500) as a viability dye then surface-labeled with fluorophore-conjugated α-CD3, α-CD4, and α-CD8 monoclonal antibodies according to manufacturer's instructions (BUV737 Mouse Anti-Human CD3; clone #UCHT-1, BD 612750. PE anti-human CD4; clone #RPA-T4, Biolegend 300539. APC anti-human CD8; clone #SK1, Biolegend 344722). Unmixed flow data were imported into FlowJo to analyze the proliferation rate within different T cell subgroups. Viable cells that were EdU+were considered to be dividing.

In FIG. 22G, PBMCs were diluted to 10⁶/mL in RPMI and activated with 1 μg/mL LPS alone or in co-culture with WT-MSCs or eMSCs (1:10 eMSC: PBMC ratio, 10k MSC: 100k PBMC) in 96 well flat bottom, tissue-culture treated plates. Conditioned media was collected at 24 h after LPS treatment, frozen at −20° C., and analyzed using a hTNFα ELISA kit (Biolegend 430204).

qPCR

WT MSCs and eMSCs were plated in a 6 well plate at 200k cells/well in complete αMEM, then left untreated or treated with αMEM supplemented with 20 ng/ml recombinant hIFNγ (R&D Systems 10067-IF). After 24 h, cells were harvested, flash frozen with liquid nitrogen, and stored at −80° C. RNA was then extracted using PureLink™ RNA Mini Kit (Invitrogen 12183018A). Reverse transcription was performed using SuperScript™ III Platinum™ One-Step qRT-PCR System (Invitrogen 11732-020) according to manufacturer's protocol on a BioRad C1000 Touch™ Thermal Cycler with a CFX96™ Real-Time System. Gene expression was measured using TaqMan® Gene Expression Assays with primers for IDO-1 (Hs00984148_ml) and GAPDH (Hs02758991_g1) (Applied Biosystems). As IDO-1 expression was not detected in untreated MSCs, IDO-1 expression was reported as a ratio of IDO-1 Cq/GAPDH Cq in lieu of 2-44Ct analysis.

### PGE2 ELISA

WT MSCs and eMSCs were plated in a 96-well, flat bottom tissue culture treated plate at 10k cells/well in complete αMEM then left untreated or treated with complete αMEM supplemented with 20 ng/ml each of recombinant hTNFα (Peprotech 300-01A-50UG), hIFNγ (R&D Systems 10067-IF), and hIL-1B (Peprotech 200-01B). After 24 h, the conditioned media was harvested and frozen at −20° C. and then analyzed using a prostaglandin E2 (PGE2) ELISA kit (Cayman 500141).

### Viral Transduction

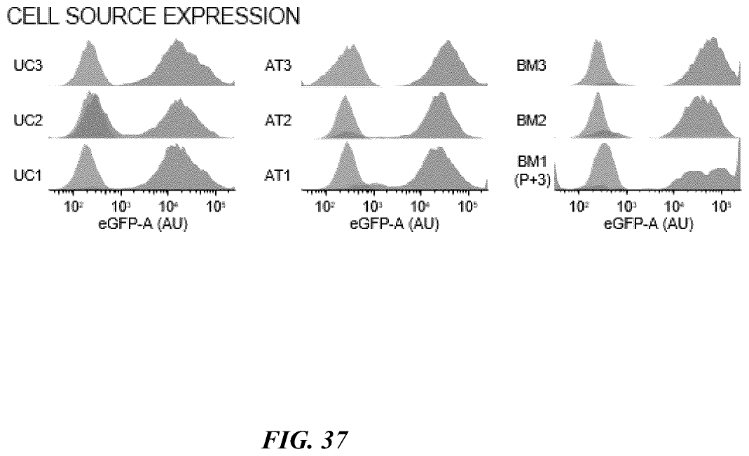

Low passage HEK-293T cells (ATCC® CRL-11268™) grown in complete Dulbecco's modified Eagle medium (DMEM) with high glucose (Gibco, 12100061) supplemented with 10% FBS, 100 units/ml penicillin, 100 μg/ml streptomycin, and 1×GlutaMax were plated in a 6 well plate at 1.3M cells per well. 1 μg of a second-generation self-inactivated transfer vector containing custom open reading frames was co-transfected with 0.7 ug gag/pol packaging vector (psPAX2, Addgene 12260) and 0.4 μg VSV-G envelope protein vector (pMD2.G, Addgene 12259) using JetPrime (Polyplus 101000046) according to manufacturer's protocol. psPAX2 and pMD2.G were gifts from Didier Trono71. Media were changed 4 h after transfection, and viral supernatant was collected at 24 and 48 h, then filter sterilized through a 0.45 μm PVDF filter (Millipore Sigma SLHVR13SL). the manufactured virus was functionally titered⁶⁹. 850k HEK-293T cells were plated per well in a 6-well plate then transduced at 60% confluence using a variable volume of viral supernatant supplemented with polybrene at a final concentration of 8 μg/mL. Transduced cells were passaged 1:5 24 h after transduction, then harvested at 48 h after transduction. The percentage of GFP+ cells were measured using flow cytometry, then plotted as GFP+ cell counts per μL virus supernatant. Linear regression was used to determine the number of GFP+ cells/μL virus using the linear region at the lower transduction efficiencies (FIG. 37E). Assuming 1 viral particle per GFP+ cell in this region, this value was converted to number of viral particles per μL and used to convert the titer to MOI by multiplying the number of viral particles per μL by the volume viral supernatant added to a known number of cells, which was then plotted as % GFP+ for each MOI (FIG. 37F).

MSCs were transduced as previously described⁵⁶. Briefly, 100k MSCs were plated per well in a 6-well plate in complete αMEM. 24 h later, the appropriate volume of virus was added to achieve the target MOI (1-20) with a final concentration of 10 μg/mL protamine sulfate (Millipore Sigma P4020-1G). For MOI 50, 35k MSCs were plated per well in a 12 well plate, to which the appropriate volume of virus was added to the target MOI with 10 μg/mL protamine sulfate. Transduced cells were harvested at 48 hours, counted, and transduction efficiency was quantified by flow cytometry. eMSCs transduced with an MOI of 10-50 were selected with puromycin and expanded for two subsequent passages, quantifying cell count and expression homogeneity at each passage.

### Statistical Analysis

Comparisons between means of each group were made with the use of one-way ANOVA followed by Turkey's correction test for multiple comparisons using Prism (GraphPad). Values at p<0.05 were considered significant.

### REFERENCES CITED IN THIS EXAMPLE

1 Bashor, C. J., Hilton, I. B., Bandukwala, H., Smith, D. M. & Veiseh, O. Engineering the next generation of cell-based therapeutics. Nat Rev Drug Discov 21, 655-675, doi:10.1038/s41573-022-00476-6 (2022).
2 Braendstrup, P., Levine, B. L. & Ruella, M. The long road to the first FDA-approved gene therapy: chimeric antigen receptor T cells targeting CD19. Cytotherapy 22, 57-69, doi:10.1016/j.jcyt.2019.12.004 (2020).
3 Stabler, C. L. & Russ, H. A. Regulatory approval of islet transplantation for treatment of type 1 diabetes: Implications and what is on the horizon. Mol Ther 31, 3107-3108, doi:10.1016/j.ymthe.2023.10.011 (2023).
4 Levy, O. et al. Shattering barriers toward clinically meaningful MSC therapies. Sci Adv 6, eaba6884, doi:10.1126/sciadv.aba6884 (2020).
5 Kitada, T., DiAndreth, B., Teague, B. & Weiss, R. Programming gene and engineered-cell therapies with synthetic biology. Science 359, doi:10.1126/science.aad1067 (2018).
6 Aijaz, A. et al. Biomanufacturing for clinically advanced cell therapies. Nat Biomed Eng 2, 362-376, doi:10.1038/s41551-018-0246-6 (2018).
7 Irving, M., Lanitis, E., Migliorini, D., Ivics, Z. & Guedan, S. Choosing the Right Tool for Genetic Engineering: Clinical Lessons from Chimeric Antigen Receptor-T Cells. Hum Gene Ther 32, 1044-1058, doi:10.1089/hum.2021.173 (2021).
8 Khang, M. et al. Manufacturing innovation to drive down cell therapy costs. Trends Biotechnol 41, 1216-1219, doi:10.1016/j.tibtech.2023.04.006 (2023).
9 De Luca, M. & Cossu, G. Cost and availability of novel cell and gene therapies: Can we avoid a catastrophic second valley of death?: Can we avoid a catastrophic second valley of death? EMBO Rep 24, e56661, doi:10.15252/embr.202256661 (2023).
10 Pittenger, M. F. et al. Mesenchymal stem cell perspective: cell biology to clinical progress. NPJ Regen Med 4, 22, doi:10.1038/s41536-019-0083-6 (2019).
11 Hare, J. M. et al. Comparison of allogeneic vs autologous bone marrow-derived mesenchymal stem cells delivered by transendocardial injection in patients with ischemic cardiomyopathy: the POSEIDON randomized trial. JAMA 308, 2369-2379, doi:10.1001/jama.2012.25321 (2012).
12 Ankrum, J. A., Ong, J. F. & Karp, J. M. Mesenchymal stem cells: immune evasive, not immune privileged. Nat Biotechnol 32, 252-260, doi:10.1038/nbt.2816 (2014).
13 Hoang, D. M. et al. Stem cell-based therapy for human diseases. Signal Transduct Target Ther 7, 272, doi:10.1038/s41392-022-01134-4 (2022).
14 Galipeau, J. & Sensebe, L. Mesenchymal Stromal Cells: Clinical Challenges and Therapeutic Opportunities. Cell Stem Cell 22, 824-833, doi:10.1016/j.stem.2018.05.004 (2018).
15 Caplan, H. et al. Mesenchymal Stromal Cell Therapeutic Delivery: Translational Challenges to Clinical Application. Front Immunol 10, 1645, doi:10.3389/fimmu.2019.01645 (2019).
16 Damasceno, P. K. F. et al. Genetic Engineering as a Strategy to Improve the Therapeutic Efficacy of Mesenchymal Stem/Stromal Cells in Regenerative Medicine. Front Cell Dev Biol 8, 737, doi:10.3389/fcell.2020.00737 (2020).
17 Ullah, M., Liu, D. D. & Thakor, A. S. Mesenchymal Stromal Cell Homing: Mechanisms and Strategies Improvement. for iScience 15, 421-438, doi:10.1016/j.isci.2019.05.004 (2019).
18 Ocansey, D. K. W. et al. Improved therapeutics of modified mesenchymal stem cells: an update. J Transl Med 18, 42, doi:10.1186/s12967-020-02234-x (2020).
19 Nowakowski, A., Walczak, P., Janowski, M. & Lukomska, B. Genetic Engineering of Mesenchymal Stem Cells for Regenerative Medicine. Stem Cells Dev 24, 2219-2242, doi:10.1089/scd.2015.0062 (2015).
20 Sage, E. K., Thakrar, R. M. & Janes, S. M. Genetically modified mesenchymal stromal cells in cancer therapy. Cytotherapy 18, 1435-1445, doi:10.1016/j.jcyt.2016.09.003 (2016).
21 Varkouhi, A. K. et al. Genetically Modified Mesenchymal Stromal/Stem Cells: Application in Critical Illness. Stem Cell Rev Rep 16, 812-827, doi:10.1007/s12015-020-10000-1 (2020).
22 Nowakowski, A., Walczak, P., Lukomska, B. & Janowski, M. Genetic Engineering of Mesenchymal Stem Cells to Induce Their Migration and Survival. Stem Cells Int 2016, 4956063, doi:10.1155/2016/4956063 (2016).
23 Hodgkinson, C. P., Gomez, J. A., Mirotsou, M. & Dzau, V. J. Genetic engineering of mesenchymal stem cells and its application in human disease therapy. Hum Gene Ther 21, 1513-1526, doi:10.1089/hum.2010.165 (2010).
24 Siraj, Y., Galderisi, U. & Alessio, N. Senescence induces fundamental changes in the secretome of mesenchymal stromal cells (MSCs): implications for the therapeutic use of MSCs and their derivates. Front Bioeng Biotechnol 11, 1148761, doi:10.3389/fbioe.2023.1148761 (2023).
25 Jiang, T. et al. In vitro expansion impaired the stemness of early passage mesenchymal stem cells for treatment of cartilage defects. Cell Death Dis 8, e2851, doi:10.1038/cddis.2017.215 (2017).
26 Mastrolia, I. et al. Challenges in Clinical Development of Mesenchymal Stromal/Stem Cells: Concise Review. Stem Cells Transl Med 8, 1135-1148, doi:10.1002/sctm.19-0044 (2019).
27 Prockop, D. J. et al. Defining the risks of mesenchymal stromal cell therapy. Cytotherapy 12, 576-578, doi:10.3109/14653249.2010.507330 (2010).
28 Schlimgen, R. et al. Risks Associated With Lentiviral Vector Exposures and Prevention Strategies. J Occup Environ Med 58, 1159-1166, doi:10.1097/JOM.0000000000000879 (2016)
29 Schambach, A., Morgan, M. & Fehse, B. Two cases of T cell lymphoma following Piggybac-mediated CAR T cell therapy. Mol Ther 29, 2631-2633, doi:10.1016/j.ymthe.2021.08.013 (2021).
30 Dao, M. A., Tate, C. C., Aizman, I., McGrogan, M. & Case, C. C. Comparing the immunosuppressive potency of naive marrow stromal cells and Notch-transfected marrow stromal cells. J Neuroinflammation 8, 133, doi:10.1186/1742-2094-8-133 (2011).
31 Kawabori, M. et al. Cell Therapy for Chronic TBI: Interim Analysis of the Randomized Controlled STEMTRA Trial. Neurology 96, e1202-e1214, doi:10.1212/WNL.0000000000011450 (2021).
32 Levy, O. et al. mRNA-engineered mesenchymal stem cells for targeted delivery of interleukin-10 to sites of inflammation. Blood 122, e23-32, doi:10.1182/blood-2013-04-495119 (2013).
33 Li, Q. et al. mRNA-engineered mesenchymal stromal cells expressing CXCR2 enhances cell migration and improves recovery in IBD. Mol Ther Nucleic Acids 26, 222-236, doi:10.1016/j.omtn.2021.07.009 (2021).
34 Dembinski, J. L. et al. Tumor stroma engraftment of gene-modified mesenchymal stem cells as anti-tumor therapy against ovarian cancer. Cytotherapy 15, 20-32, doi:10.1016/j.jcyt.2012.10.003 (2013).
35 von Einem, J. C. et al. Treatment of advanced gastrointestinal cancer with genetically modified autologous mesenchymal stem cells: Results from the phase 1/2 TREAT-ME-1 trial. Int J Cancer 145, 1538-1546, doi:10.1002/ijc.32230 (2019).
36 Mohme, M. et al. Local Intracerebral Immunomodulation Using Interleukin-Expressing Mesenchymal Stem Cells in Glioblastoma. Clin Cancer Res 26, 2626-2639, doi:10.1158/1078-0432.CCR-19-0803 (2020).
37 Gonzalez-Junca, A. et al. SENTI-101, a Preparation of Mesenchymal Stromal Cells Engineered to Express IL12 and IL21, Induces Localized and Durable Antitumor Immunity in Preclinical Models of Peritoneal Solid Tumors. Mol Cancer Ther 20, 1508-1520, doi:10.1158/1535-7163.MCT-21-0030 (2021).
38 Hamann, A., Nguyen, A. & Pannier, A. K. Nucleic acid delivery to mesenchymal stem cells: a review of nonviral methods and applications. J Biol Eng 13, 7, doi:10.1186/s13036-019-0140-0 (2019).
39 Hamann, A. & Pannier, A. K. Innovative nonviral gene delivery strategies for engineering human mesenchymal stem cell phenotypes toward clinical applications. Curr Opin Biotechnol 78, 102819, doi:10.1016/j.copbio.2022.102819 (2022).
40 Wang, X. & Marchisio, M. A. Synthetic polycistronic sequences in eukaryotes. Synth Syst Biotechnol 6, 254-261, doi:10.1016/j.synbio.2021.09.003 (2021).
41 Harting, M., Jimenez, F., Pati, S., Baumgartner, J. & Cox, C., Jr. Immunophenotype characterization of rat mesenchymal stromal cells. Cytotherapy 10, 243-253, doi:10.1080/14653240801950000 (2008).
42 Timmins, L. M. et al. Selecting a Cell Engineering Methodology During Cell Therapy Product Development. Cell Transplant 30, 9636897211003022, doi:10.1177/09636897211003022 (2021).
43 Kabat, M., Bobkov, I., Kumar, S. & Grumet, M. Trends in mesenchymal stem cell clinical trials 2004-2018: Is efficacy optimal in a narrow dose range? Stem Cells Transl Med 9, 17-27, doi:10.1002/sctm.19-0202 (2020).
44 Weber, E., Engler, C., Gruetzner, R., Werner, S. & Marillonnet, S. A modular cloning system for standardized assembly of multigene constructs. PLOS One 6, e16765, doi:10.1371/journal.pone.0016765 (2011).
45 Yusa, K., Zhou, L., Li, M. A., Bradley, A. & Craig, N. L. A hyperactive piggyBac transposase for mammalian applications. Proc Natl Acad Sci USA 108, 1531-1536, doi:10.1073/pnas. 1008322108 (2011).
46 Hass, R., Kasper, C., Bohm, S. & Jacobs, R. Different populations and sources of human mesenchymal stem cells (MSC): A comparison of adult and neonatal tissue-derived MSC. Cell Commun Signal 9, 12, doi:10.1186/1478-811X-9-12 (2011).
47 Sanz-Nogues, C. & O'Brien, T. Current good manufacturing practice considerations for mesenchymal stromal cells as therapeutic agents. Biomater Biosyst 2, 100018, doi:10.1016/j.bbiosy.2021.100018 (2021).
48 Phinney, D. G., Galipeau, J., Msc Committee Of The International Society Of, C. & Gene, T. Manufacturing mesenchymal stromal cells for clinical applications: A survey of Good Manufacturing Practices at U.S. academic centers. Cytotherapy 21, 782-792, doi:10.1016/j.jcyt.2019.04.003 (2019).
49 De La Vega, J., Braak, B. T., Azzoni, A. R., Monteiro, G. A. & Prazeres, D. M. Impact of plasmid quality on lipoplex-mediated transfection. J Pharm Sci 102, 3932-3941, doi:10.1002/jps.23709 (2013).
50 Goncalves, G. A., Bower, D. M., Prazeres, D. M., Monteiro, G. A. & Prather, K. L. Rational engineering of Escherichia coli strains for plasmid biopharmaceutical manufacturing. Biotechnol J 7, 251-261, doi:10.1002/biot.201100062 (2012).
51 Kelly, A. M., Plautz, S. A., Zempleni, J. & Pannier, A. K. Glucocorticoid Cell Priming Enhances Transfection Outcomes in Adult Human Mesenchymal Stem Cells. Mol Ther 24, 331-341, doi:10.1038/mt.2015.195 (2016).
52 Tristan, C. A. et al. Efficient and safe single-cell cloning of human pluripotent stem cells using the CEPT cocktail. Nat Protoc 18, 58-80, doi:10.1038/s41596-022-00753-z (2023).
53 Skiles, M. L., Marzan, A. J., Brown, K. S. & Shamonki, J. M. Comparison of umbilical cord tissue-derived mesenchymal stromal cells isolated from cryopreserved material and extracted by explantation and digestion methods utilizing a split manufacturing model. Cytotherapy 22, 581-591, doi:10.1016/j.jcyt.2020.06.002 (2020).
54 Ma, L. X., Levitan, D. & Baergen, R. N. Weights of Fetal Membranes and Umbilical Cords: Correlation With Placental Pathology. Pediatr Dev Pathol 23, 249-252, doi:10.1177/1093526619889460 (2020).
55 Maude, S. L. et al. Tisagenlecleucel in Children and Young Adults with B-Cell Lymphoblastic Leukemia. N Engl J Med 378, 439-448, doi:10.1056/NEJMoa1709866 (2018).
56 Lin, P. et al. Efficient lentiviral transduction of human mesenchymal stem cells that preserves proliferation and differentiation capabilities. Stem Cells Transl Med 1, 886-897, doi:10.5966/sctm.2012-0086 (2012).
57 Saxena, A. et al. Interleukin-10 paradox: A potent immunoregulatory cytokine has been difficult to harness for immunotherapy. Cytokine 74, 27-34, that doi:10.1016/j.cyto.2014.10.031 (2015).
58 Pires, I. S., Hammond, P. T. & Irvine, D. J. Engineering Strategies for Immunomodulatory Cytokine Therapies-Challenges and Clinical Progress. Adv Ther (Weinh) 4, doi:10.1002/adtp.202100035 (2021).
59 Chen, M. et al. Long-term monitoring of intravital biological processes using fluorescent protein-assisted NIR-II imaging. Nat Commun 13, 6643, doi:10.1038/s41467-022-34274-w (2022).
60 Skiles, M. L., Brown, K. S., Tatz, W., Swingle, K. & Brown, H. L. Quantitative analysis of composite umbilical cord tissue health using a standardized explant approach and an assay of metabolic activity. Cytotherapy 20, 564-575, doi:10.1016/j.jcyt.2018.01.001 (2018).
61 Smith, J. R. et al. Standardizing Umbilical Cord Mesenchymal Stromal Cells for Translation to Clinical Use: Selection of GMP-Compliant Medium and a Simplified Isolation Method. Stem Cells Int 2016, 6810980, doi:10.1155/2016/6810980 (2016).
62 George, M. J. et al. Clinical Cellular Therapeutics Accelerate Clot Formation. Stem Cells Transl Med 7, 731-739, doi:10.1002/sctm.18-0015 (2018).
63 Kota, D. J. et al. Prostaglandin E2 Indicates Therapeutic Efficacy of Mesenchymal Stem Cells in Experimental Traumatic Brain Injury. Stem Cells 35, 1416-1430, doi:10.1002/stem.2603 (2017).
64 Sekiya, I. et al. Expansion of human adult stem cells from bone marrow stroma: conditions that maximize the yields of early progenitors and evaluate their quality. Stem Cells 20, 530-541, doi:10.1634/stemcells.20-6-530 (2002).
65 Wiese, D. M., Ruttan, C. C., Wood, C. A., Ford, B. N. & Braid, L. R. Accumulating Transcriptome Drift Precedes Cell Aging in Human Umbilical Cord-Derived Mesenchymal Stromal Cells Serially Cultured to Replicative Senescence. Stem Cells Transl Med 8, 945-958, doi:10.1002/sctm.18-0246 (2019).
66 Vidal, M. A. et al. Cell growth characteristics and differentiation frequency of adherent equine bone marrow-derived mesenchymal stromal cells: adipogenic and osteogenic capacity. Vet Surg 35, 601-610, doi:10.1111/j.1532-950X.2006.00197.x (2006).
67 Chung, C. T., Niemela, S. L. & Miller, R. H. One-step preparation of competent Escherichia coli: transformation and storage of bacterial cells in the same solution. Proc Natl Acad Sci USA 86, 2172-2175, doi:10.1073/pnas.86.7.2172 (1989).
68 Kota, D. J. et al. Differential MSC activation leads to distinct mononuclear leukocyte binding mechanisms. Sci Rep 4, 4565, doi:10.1038/srep04565 (2014).
69 Zufferey, R. et al. Self-inactivating lentivirus vector for safe and efficient in vivo gene delivery. J Virol 72, 9873-9880, doi:10.1128/JVI.72.12.9873-9880.1998 (1998).

### Example 7

### Problem: Traumatic Brain Injury (TBI)

There are 2.5 million traumatic brain injuries (TBIs) each year in the United States. TBI is the leading cause of death and disability among children and military service members. There are lasting impacts that lead to long term disability. For example, survivors typically need long-term care, including physical therapy, and can expect a lifetime of challenges. These disabilities disproportionally affect children. TBI-associated burden on caregivers and the healthcare system is estimated to cost approximately $75 billion per year.

Primary injury causes irreversible tissue damage. Secondary injury from brain inflammation causes further damage and can lead to disability. Currently, the standard of care includes symptom management. This standard of care is fundamentally palliative, and only addresses symptoms of secondary injury through surgery, anti-seizure and diuretic medications, or coma inductions.

Current cell therapies can treat the neuroinflammation and help repair tissue damage. Mesenchymal stem/stromal cells (MSCs) are under investigation to treat TBI but fail in clinical trials due to poor homing to the injury and insufficient potency.

### TBI Market Overview

### Total:

$3 Billion-TAM: Global, 69 million patients per year

### Addressable:

$1 Billion-SAM: USA, 2.5 million patients per year

### Obtainable:

$72 Million-SOM: Texas, 1445 patients per year

### Why Traumatic Brain Injury?

1. Significant impact in patient population, with the potential to improve survival and quality of life
2. No approved therapy and an insufficient standard of care
3. Potential for FDA Breakthrough and/or Fast Track designations streamlines regulatory path
4. Validated TBI animal model in hand
5. Strong clinical network in TMC with clinical protocols in place for rapid deployment
6. Access to funding from Department of Defense for R&D and future clinical trials

### Platform Described Herein Allows Custom Cell Therapies

Cells are engineerable, accessible and scalable. The platform is able to get the ceels where they need to go. Further, the platform enhances cells with Tx payload.

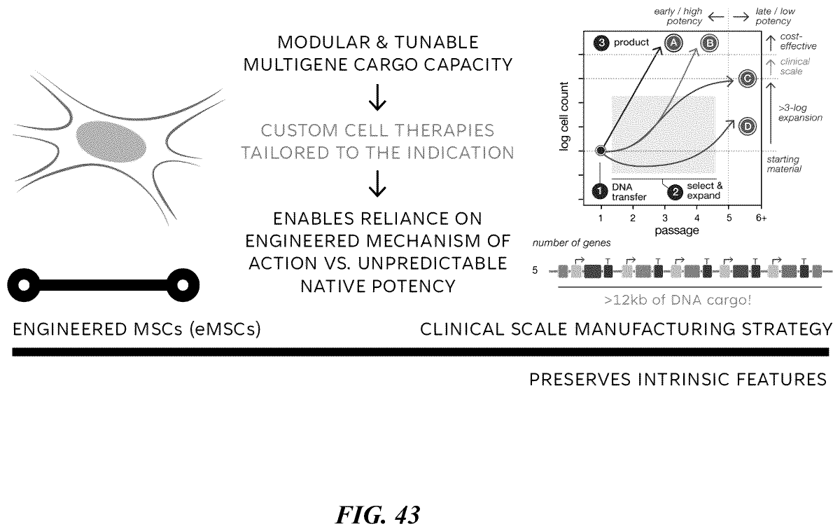

Referring to FIG. 43., data showing the engineerable platform and scalable assembly line described herein is provided.

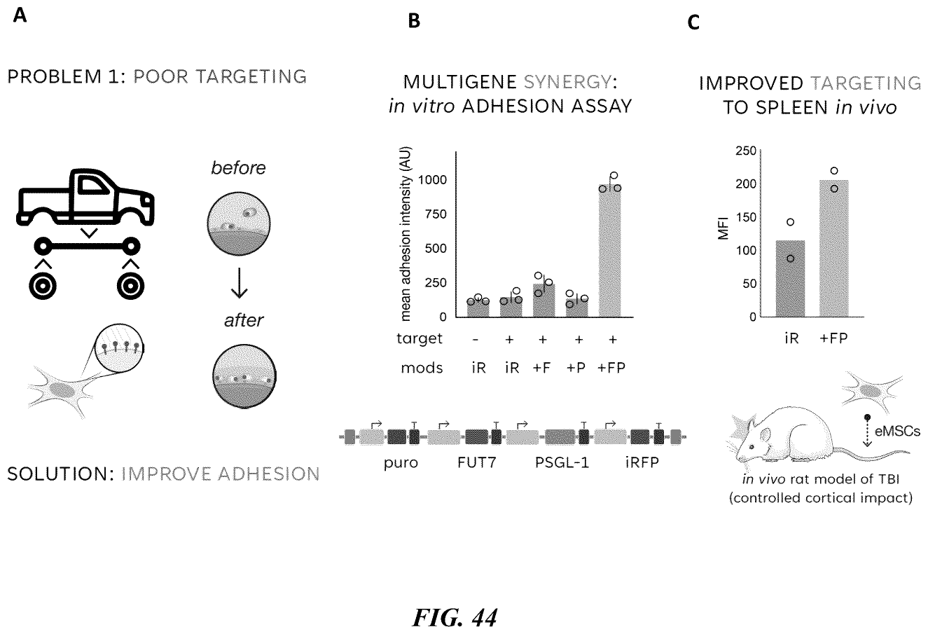

Referring to FIG. 44, data showing leveraging synthetic biology to overcome challenges with MSCs is provided. FIG. 44A provides a schematic showing poor targeting and improved adhesion. FIG. 44B provides data showing multigene synergy using an in vitro adhesion assay. FIG. 44C provides data showing improved targeting to the spleen in vivo.

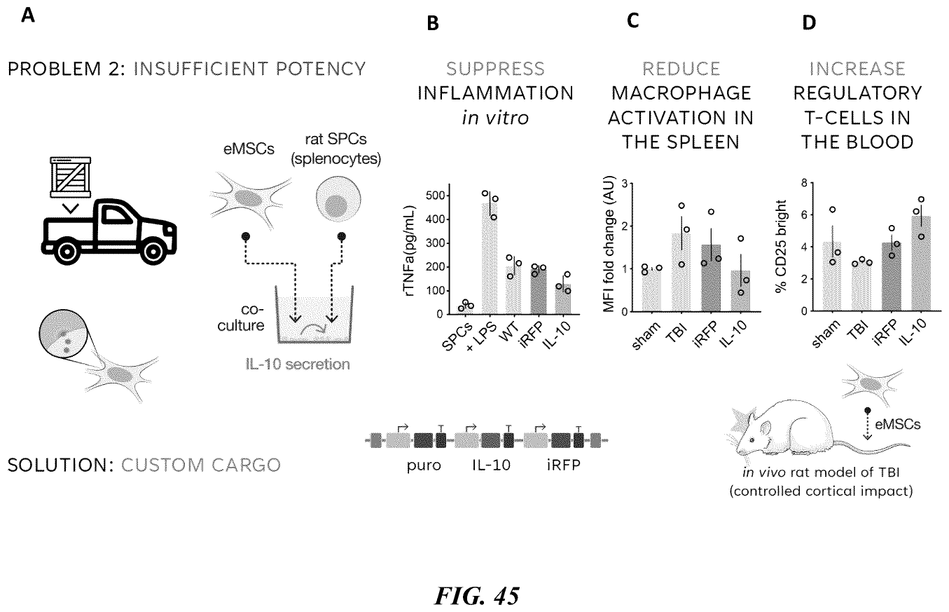

Referring to FIG. 45, data showing leveraging synthetic biology to overcome challenges with MSCs is provided. FIG. 45A provides a schematic showing insufficient potency and custom cargo. FIG. 45B provides data showing suppression of inflammation in vitro. FIG. 45C provides data showing reduction of macrophage activation in the spleen. FIG. 45D provides data showing an increase in regulatory T-cells in the blood.

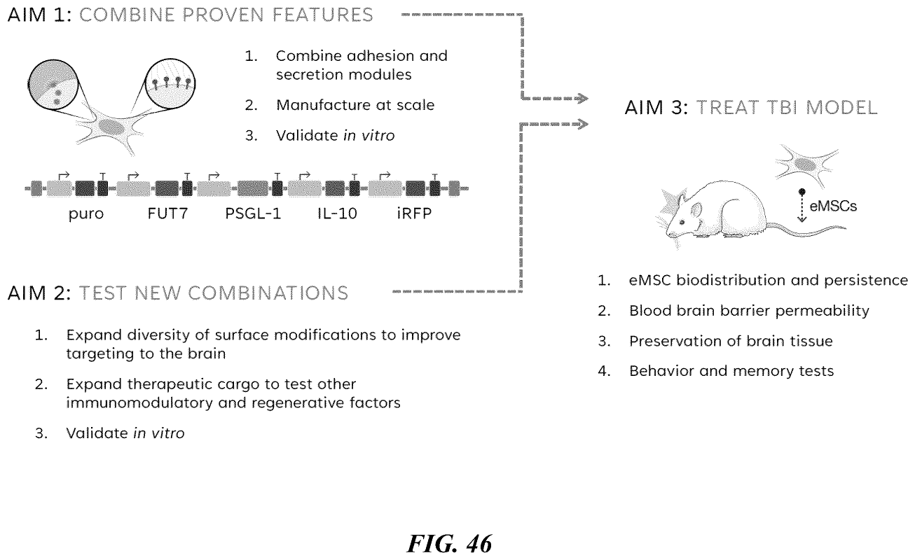

Referring to FIG. 46, a schematic for treating a TBI model using eMSCs is provided.

Our vision is to build next-generation cell therapies on the chassis:
Program Biodistribution: Improve targeting and persistence in target compartments
Deploy Sense and Respond: This includes: 1) orthogonal fast time-scale sense and respond; 2) cell state sensing via synthetic promoters; and 3) inducible kill switch for safety

### Secrete Multiple Factors:

Stroke?—Anti-inflammatory and pro-regenerative cocktail
Cancer?—Cytotoxic and pro-inflammatory combination only in the tumor
Wounds?—Enzymes and tissue factors to improve clotting

### Leverage Exosomes:

Option 1: Long distance delivery of therapeutic cargo distal from the cells
Option 2: Standalone therapeutic

### Value Proposition

### Patient:

Improve patient outcomes—Reduce secondary injury due to brain inflammation Reduce disability and time in the hospital—ease cost burden on healthcare system
Increase survivor independence

### Technology:

Our cell chassis is intrinsically non-destructive, allowing an allogenic cell therapy that won't hurt the patient.
The cells can be engineered with custom genetic cargo, turning them into super immune regulators or stealthy Trojan horses.

### Initial Markets for Lead Candidate:

### Pediatric TBI Patients:

Existing structure for clinical trials with academic partner
Significant benefits of long-term quality of life improvements for the patient population

Subsequent clinical trials for adult TBI patients can build on pediatric data. Further, the mechanism of action of the lead candidate can also benefit stroke patients.

### Example 8

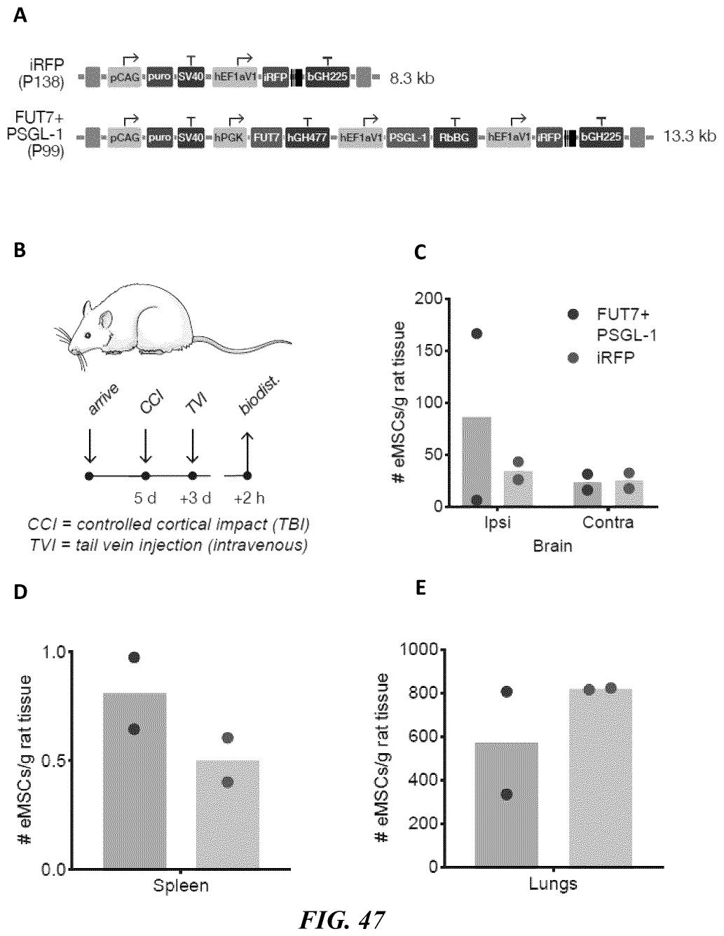

Referring to FIG. 47, data showing in vivo experiment with FUT7+PSGL-1 eMSCs after TBI is provided. Here, we evaluated multiple expression levels of FUT7 and PSGL-1 and found a construct that expressed well, manufactured well, and improved adhesion. We found that FUT7+PSGL-1 may enhance targeting to brain and spleen, improve escape from lungs. FIG. 47A provides a schematic showing constructs P138 and P199. Construct P99 expressed well, manufactured well, and improved adhesion, and displays hPGK-FUT7 (low expression) with hEF1aV1-PSGL-1 (med expression). FIG. 47B provides a schematic showing the expansion of eMSCs programmed with P99 and infused in a rat model of traumatic brain injury via tail vein injection in a pilot experiment (n=2). FIG. 47C shows that, in one animal, the number of eMSCs in the brain was increased. FIG. 47D shows that, in both animals, the number of eMSCs in the spleen was increased (a primary mediator of inflammation after TBI and one of our primary target organs). FIG. 47E shows that, in both animals, the number of eMSCs in the lungs was decreased. This data indicates that we enhanced the escape of the eMSCs from the organ that historically traps MSCs.

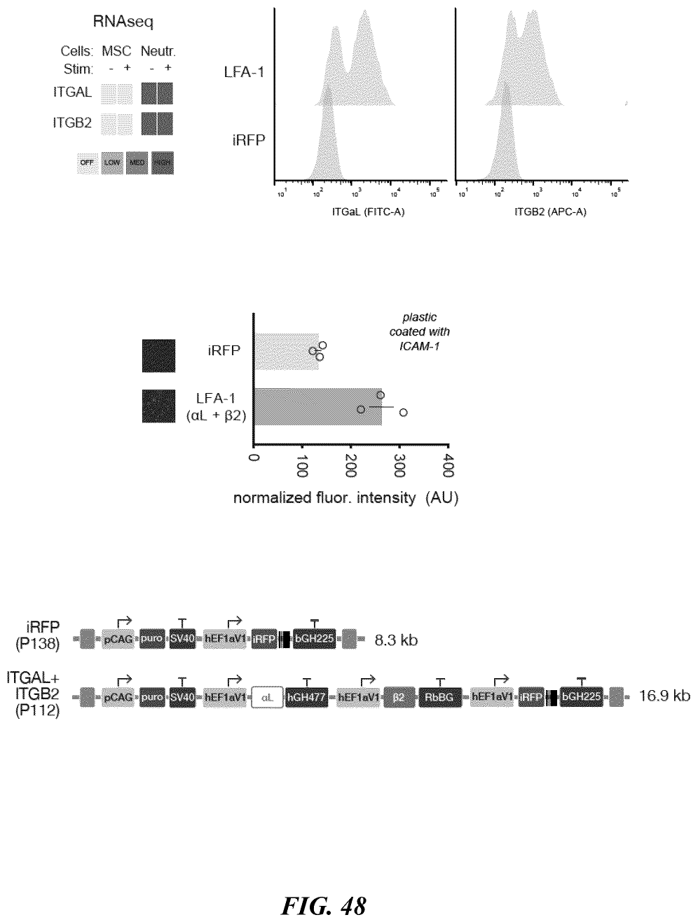

Referring to FIG. 48, data showing the expression of LFA-1 eMSCs (Integrin aL and B2) is provided. Here, we added integrins to the adhesion cascade. MSCs don't express the LFA-1 integrin pair (Integrin aL and Integrin β2). Thus, we engineered them to express this pair. We found that the combination of integrin aL and B2 enhances eMSC adhesion to ICAM-1.

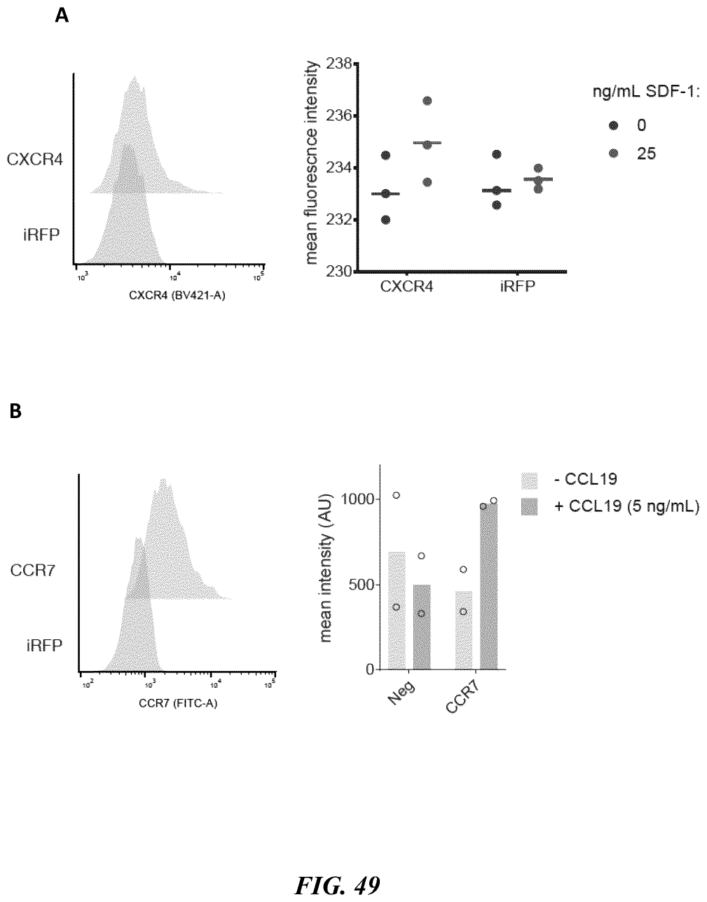

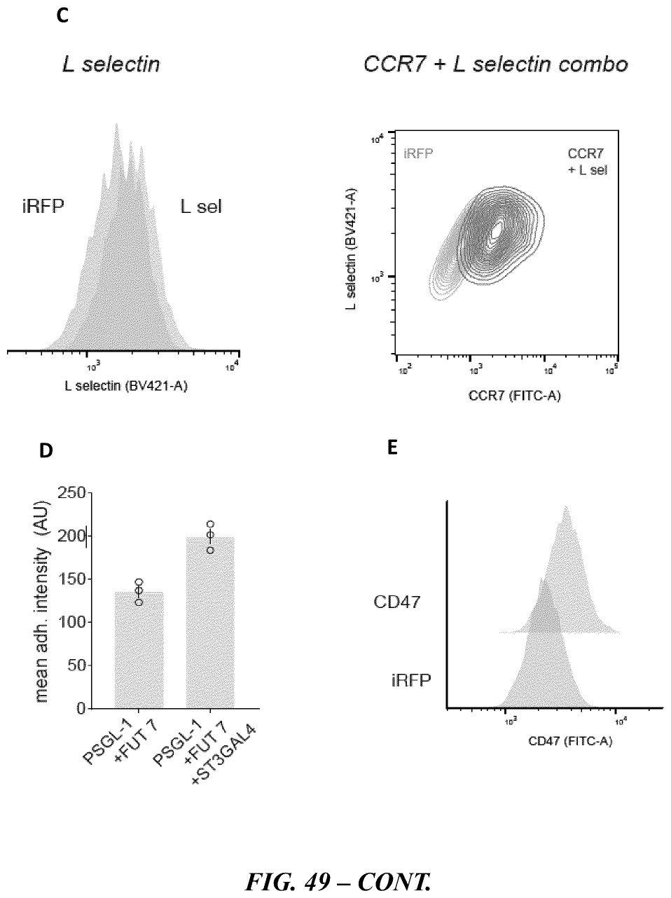

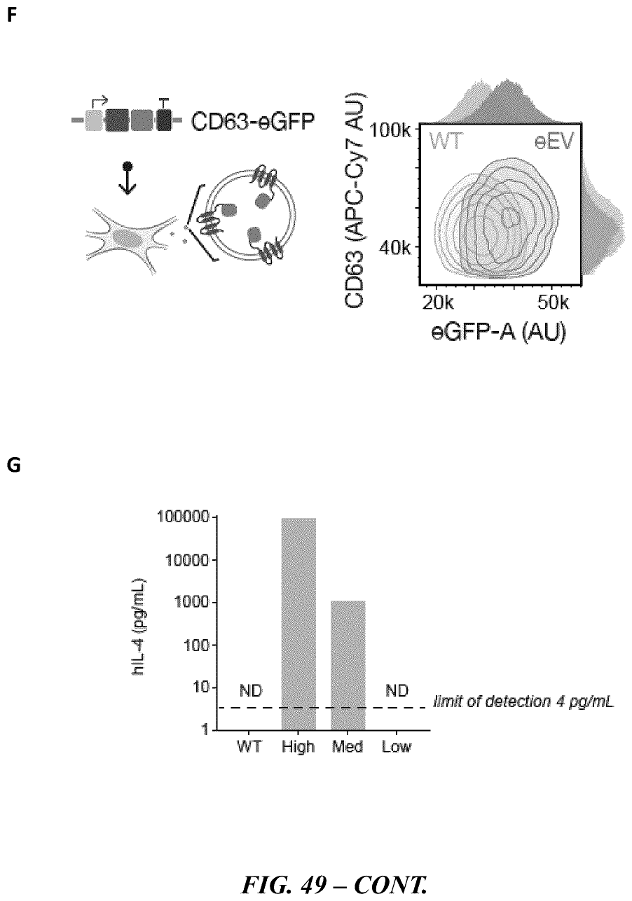

Referring to FIG. 49, data showing migration and expression of eMSCs using various modifications is provided. FIG. 49A shows that CXCR4 increases the migration of eMSCs towards an SDF-1 gradient. FIG. 49B shows that CCR7 increases the migration of eMSCs towards a CCL19 gradient. FIG. 49C shows that the combination of L-selectin and CCR7 allow eMSCs to enter the lymphatic system through the high endothelial venules. FIG. 49D shows that adding ST3GAL4 to the PSGL-1+FUT7 pair further improves adhesion. ST3GAL4 is a sialyltransferase enzyme that post-translationally modifies PSGL-1 with sialic acid, which can further enhance the affinity of PSGL-1 for P and E selectin. FIG. 49E shows eMSCs expressing CD47 can persist longer in vivo. FIG. 49F shows that CD63-eGFP results in cargo loading into extracellular vesicles. This modification allows us to load a GFP cargo into extracellular vesicles. Importantly, this was another example of a protein that was toxic to MSCs, and reducing the expression level increased the manufacturability (Table 7). FIG. 49G provides data showing tunable secretion of IL-4 is provided. IL-4 expressed well, and we were able to modulate its secretion through promoter selection, as with IL-10.

TABLE
Expression of CD63-eGFP.

| | High expr. (CMV) | Med expr. (hEF1a) |
|---|---|---|
| CD63-eGFP | P75: 2.74M | P132: 4.07M |
| PTGFRN-eGFP | P76: 632K | P133: 4.08M |

We have successfully manufactured cells expressing CD47, NQO1, and iCaspase9 individually and in combination. CD47 is a “don't eat me” signal that blocks phagocytosis by macrophages. eMSCs expressing this can persist longer in vivo. We plan to test this modification in vitro using a macrophage cell line and in vivo. NQO1 is a quinone oxidoreductase that helps with cellular detoxification and protection against oxidative stress. Upcoming experiments will validate its expression using qPCR and its function using in vitro hypoxia models. We anticipate this will enhance persistence in vivo, so we will test this in animals soon thereafter. iCaspase9 is a synthetic protein that allows for small molecule inducible apoptosis.

We are in the process of constructing or testing the following modifications: CXCR1, CX3CR1, CCR2, ITGAM, ITGA4, ITGB7, IL-13. Further, we plan to build and test the following modifications: CXCR2, CCR1, CCR3, Talin1, Kindlin3, and PECAM

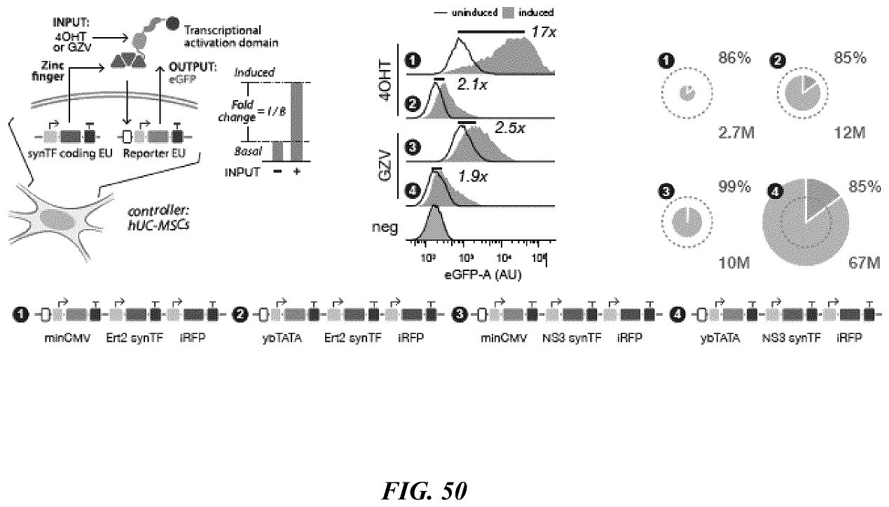

Referring to FIG. 50, data showing that the manufacturing platform can be used to evaluate synthetic circuits for manufacturability is provided. For example, without our platform we would have selected circuit #1 and moved forward to clinical development, then found out later that we couldn't manufacture cells bearing this circuit. Instead, we have learned that basal expression level is directly correlated with manufacturability (rank order manufacturability: 4 >2 >3 >1, while basal expression rank order is 4<2<3<1). Therefore, we can design circuits with low basal and high inducibility to create manufacturable cell products bearing circuits with high fold change.

### EQUIVALENTS

Those skilled in the art will recognize, or be able to ascertain, using no more than routine experimentation, numerous equivalents to the specific substances and procedures described herein. Such equivalents are considered to be within the scope of this invention and are covered by the following claims.

What is claimed:

1. A genetically engineered cell, wherein the cell is engineered to express one or more of the following:
one or more of a cell adhesion protein,
one or more of an immune modulatory or survival protein,
one or more of a small molecule inducible safety switch,
one or more of a selectable marker,
one or more of a reporter, and/or
one or more of a cargo.

2. The engineered cell of claim 1, wherein the cell comprises a mesenchymal stem/stromal cell (MSC) and its derivatives, an induced pluripotent stem cell (iPSC) and its derivatives, a neural stem cell (NSC) and its derivatives, a hematopoietic stem cell and its derivates, an ARPE-19 cell, or a human embryonic kidney 293 (HEK 293) cell.

3. The engineered cell of claim 1, wherein the cell adhesion protein allows cell homing to a target tissue.

4. The engineered cell of claim 3, wherein the target tissue comprises an inflamed and/or damaged tissue.

5. The engineered cell of claim 3, wherein the tissue comprises neuronal tissue.

6. The engineered cell of claim 1, wherein the cell adhesion protein comprises a P-selectin ligand, an E-selectin ligand, an L-selectin ligand, or a combination thereof.

7. The engineered cell of claim 6, wherein the cell expresses a P-selectin ligand, an E-selectin ligand, an L-selectin ligand, or a combination thereof at a level that exceeds the level of expression of a native population of cells.

8. The engineered cell of claim 1, wherein the cell adhesion protein comprises PSGL-1, ESL-1, CD44, CD24, PNAd, GlyCAM, CD34, L selectin, ITGB2, ITGAD, ITGAX, ITGAM, PECAM, ITGAL, ITGB1, ITGA9, ITGA4, ITGB7, LFA-1, VLA-4, MAC-1, Talin, Kindlin, FUT7, FUT6, FUT4, ST3GAL4, ST3GAL6, GCNT1, B4GALT1, TPST1, TPST2, ICAM-1, ICAM-2, LPAM-1, JAM-A, JAMB, CXCR1, CXCR2, CXCR3, CXCR4, CXCR5, CXCR6, CXCR7, CCR1, CCR2, CCR3, CCR4, CCR5, CCR6, CCR7, CCR8, CCR9, CCR10, CCR11, CX3CR1, EMR2, EMR3, CD97, MMP-1, MMP-2, MMP-9, TIMP1-3, or any combination thereof.

9. The engineered cell of claim 1, wherein the immune modulatory or survival protein comprises CCL21, CTLA4, CD39, CD73, TRAILR2, PDL1, FASL, H2-M3, IL-1R2, CD47, MFG-E8, CD200, HLA-G, HLA-E, NQO1, BCL2, Survin, or any combination thereof.

10. The engineered cell of claim 1, wherein the selectable marker allows manufacturing.

11. The engineered cell of claim 1, wherein the selectable marker comprises puromycin acetyl transferase, truncated CD34, PSGL-1, Blasticidin, or Geneticin.

12. The engineered cell of claim 1, wherein the reporter comprises iRFP713, luciferase eBFP2, eGFP, mCherry, or SEAP.

13. The engineered cell of claim 1, wherein the cargo comprises IL-4, IL-6, IL-10, IL-11, IL-35, IDO1, COX2, TSG-6, TGFB, TNFR2, IFNAR1, GDNF, BDNF, NGF, FGF1-2, NTF3, VEGF, HGF, TGFA, TRAIL, IFNB, HSF-TK, IL-13, IL-12, IL-21, or IL-2.

14. A method of manufacturing the genetically engineered cell of claim 1, the method comprising expressing therein one or more of the following:
one or more of a cell adhesion protein,
one or more of a signal regulatory protein,
one or more of a small molecule inducible safety switch,
one or more of a selectable marker,
one or more of a reporter, and/or
one or more of a cargo.

15. The method of claim 14, further comprising isolating and harvesting a cell from a subject or a biological sample.

16. The method of claim 14, further comprising engineering the cell to express one or more of the following:
one or more of a cell adhesion protein,
one or more of an immune modulatory or survival protein,
one or more of a small molecule inducible safety switch,
one or more of a selectable marker,
one or more of a reporter, and/or
one or more of a cargo.

17. The method of claim 16, further comprising selecting for the cells expressing the proteins.

18. The method of claim 14, further comprising expanding the isolated cells and/or the genetically engineered cells.

19. A method of treating a disease, disorder or medical condition manifesting as inflamed and/or damaged tissue in a subject, the method comprising administering to the subject a population of the genetically engineered cells of claim 1.

20. The method of claim 19, wherein the disease, disorder or medical condition comprises neuroinflammation.

21. The method of claim 20, wherein the disease, disorder or medical condition comprises traumatic brain injury.


SEQUENCE LISTING (49 sequences)

SEQ ID NO: 1 - protein, 317 aa, Rhodopseudomonas palustris
MAAEGSVARQPDLLTCDDEPIHIPGAIQPHGLLLALAADMTIVAGSDNLPELTGLAIGALIGRSAADVFDSETHNRLTIALAEPGAAVGAPITVGFTMRKDAGFIGSWHRHDQLIFLELEPPQRDVAEPQAFFRRTNSAIRRLQAAETLESACAAAAQEVRKITGFDRVMIYRFASDFSGEVIAEDRCAEVESKLGLHYPASTVPAQARRLYTINPVRIIPDINYRPVPVTPDLNPVTGRPIDLSFAILRSVSPVHLEFMRNIGMHGTMSISILRGERLWGLIVCHHRTPYYVDLDGRQACELVAQVLAWQIGVMEE

SEQ ID NO: 2 - protein, 240 aa, synthetic construct
MAVSKGEELFTGVVPILVELDGDVNGHKFSVRGEGEGDATNGKLTLKFICTTGKLPVPWPTLVTTLSHGVQCFARYPDHMKQHDFFKSAMPEGYVQERTIFFKDDGTYKTRAEVKFEGDTLVNRIELKGVDFKEDGNILGHKLEYNFNSHNIYIMAVKQKNGIKVNFKIRHNVEDGSVQLADHYQQNTPIGDGPVLLPDSHYLSTQSVLSKDPNEKRDHMVLLEFRTAAGITLGMDELYK

SEQ ID NO: 3 - protein, 241 aa, synthetic construct
MAMVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 4 - protein, 240 aa, synthetic construct
MAGNSVSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWQASSERMYPEDGALKGEIKQRLKLKDGGHYDAEVKTTYKAKKPVQLPGAYNVNIKLDITSHNEDYTIVEQYERAEGRHSTGGMDELYK

SEQ ID NO: 5 - protein, 171 aa, synthetic construct
MVFTLEDFVGDWRQTAGYNLDQVLEQGGVSSLFQNLGVSVTPIQRIVLSGENGLKIDIHVIIPYEGLSGDQMGQIEKIFKVVYPVDDHHFKVILHYGTLVIDGVTPNMIDYFGRPYEGIAVFDGKKITVTGTLWNGNKIIDERLINPDGSLLFRVTINGVTGWRLCERILA

SEQ ID NO: 6 - protein, 520 aa, synthetic construct
MVLGPCMLLLLLLLGLRLQLSLGIIPVEEENPDFWNREAAEALGAAKKLQPAQTAAKNLIIFLGDGMGVSTVTAARILKGQKKDKLGPEIPLAMDRFPYVALSKTYNVDKHVPDSGATATAYLCGVKGNFQTIGLSAAARFNQCNTTRGNEVISVMNRAKKAGKSVGVVTTTRVQHASPAGTYAHTVNRNWYSDADVPASARQEGCQDIATQLISNMDIDVILGGGRKYMFRMGTPDPEYPDDYSQGGTRLDGKNLVQEWLAKRQGARYVWNRTELMQASLDPSVTHLMGLFEPGDMKYEIHRDSTLDPSLMEMTEAALRLLSRNPRGFFLFVEGGRIDHGHHESRAYRALTETIMFDDAIERAGQLTSEEDTLSLVTADHSHVFSFGGYPLRGSSIFGLAPGKARDRKAYTVLLYGNGPGYVLKDGARPDVTESESGSPEYRQQSAVPLDEETHAGEDVAVFARGPQAHLVHGVQEQTFIAHVMAFAACLEPYTACDLAPPAGTTDAAHPGRSRSKRLD

SEQ ID NO: 7 - DNA, 7582 bp, synthetic construct
  source 1..7582: mol_type = other DNA
tttttaaccctagaaagatagtctgcgtaaaattgacgcatgcattcttgaaatattgctctctctttctaaatagcgcgaatccgtcgctgtgcatttaggacatctcagtcgccgcttggagctcccgtgaggcgtgcttgtcaatgcggtaagtgtcactgattttgaactataacgaccgcgtgagtcaaaatgacgcatgattatcttttacgtgacttttaagatttaactcatacgataattatattgttatttcatgttctacttacgtgataacttattatatatatattttcttgttatagatatcatcaactttgtatagaaaagttgctcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattgcaagtttgtacaaaaaagcaggctgaatgccgccaccatggctaccgagtataaaccgacggtccgacttgcgactcgggatgacgttccacgcgccgtgaggactctggccgccgccttcgctgactacccggccacacggcacacagttgaccccgacagacacatagagcgggtcacagagttgcaagaacttttcctgacgcgggttggactcgatattggaaaagtttgggttgccgacgatggtgccgcagttgcggtgtggactactccagaaagtgtagaagctggagccgtattcgctgaaatagggccacggatggcagagctgtctggtagcaggctcgcagcgcagcagcagatggaaggccttctcgctccgcatcggcctaaggaaccggcgtggttcctggctacggtgggagtctcacctgaccatcaaggaaagggattgggaagtgctgtcgttcttccaggcgtggaagcggccgaacgagcaggcgtcccagccttcttggaaacgagcgcacccaggaatcttccattctacgagcgactgggtttcactgtgacagcagacgtagaagtccctgaagggcctcggacttggtgcatgaccagaaagcctggggcttgaaacgccatgatccagacatgataagatacattgatgagtttggacaaaccacaactagaatgcagtgaggcgcgcctggatacttctgcctgcaggggagatgtcttccaaattgacggctagctcagtcctaggtacagtgctagcataccagataagttcgtcggtcggttatgcagtttaaggtttacacctataaaagagagagccgttatcgtctgtttgtggatgtacagagtgatattattgacacgcccgggcgacggatggtgatccccctggccagtgcacgtctgctgtcagataaagtctcccgtgaactttacccggtggtgcatatcggggatgaaagctggcgcatgatgaccaccgatatggccagtgtgccggtgtccgttatcggggaagaagtggctgatctcagccaccgcgaaaatgacatcaaaaacgccattaacctgatgttctggggaatataataatggcctcggtaccaaattccagaaaagaggcctcccgaaaggggggccttttttcgttttggtccgtggcggaccaaaacgaaaaaagacgcttttcagcgtcttattgttcgtctttggtaccgaggccattatttgtatagttcatccatgccaccggtagaatgacgaccctccgcgcgctcatattgctctacgatcgtataatcttcattatgagataagatgtccagtttaatatccacattgtacgcgccaggcagctgcacaggtttcttggctttgtacgtggttttcacttcagcgtcataatggccgccatctttcagtttcaggcgctgtttaatttcgcctttcagcgcaccatcttccggatacatgcgttcggtagacgcctcccaacccatcgtcctacactgcatcaccgggccatcagatggaaaattagtaccacgcagtttaactttatagatgaactcgccatcctgaagggaggagtcctgagtgacggtcacgacaccaccatcttcaaaattcattacgcgttcccagttgaaaccttccggaaaagacagcttcagatagtccgggatatccgctgggtgtttaacatacgctttagaaccgtacataaattgcgggctcaggatgtcccacgcaaaaggcagcgggccgcctttagtcactttcagtttggcggtctgggtgccttcatacggacggccctcgccttcgccttcgatctcgaactcgtgaccgttaacagaaccctccatgtgaactttgaagcgcatgaactctttaatgatagctaagttgttttcttctcctttactcaccatttagtatttctcctctttcactaggtcgagtagctagcataatacctaggactgagctagctgtaaagaaggaagacatggcagcggccgcgcctgggcttacttaattaacgagagcataatattgatatgtgccaaagttgtttctgactgactaataagtataatttgtttctattatgtataggttaagctaattacttattttataatacaacatgactgtttttaaagtacaaaataagtttatttttgtaaaagagagaatgtttaaaagttttgttactttatagaagaaattttgagtttttgtttttttttaataaataaataaacataaataaattgtttgttgaatttattattagtatgtaagtgtaaatataataaaacttaatatctattcaaattaataaataaacctcgatatacagaccgataaaacacatgcgtcaattttacgcatgattatctttaacgtacgtcacaatatgattatctttctagggttaaataatagtttctaatttttttattattcagcctgctgtcgtgaataccgagctccaattcgccctatagtgagtcgtattacaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctcgaaattaaccctcactaaagggaacaaaagctggtacctcgcgcgacttggtttgccattctttagcgcgcgtcgcgtcacacagcttggccacaatgtggtttttgtcaaacgaagattctatgacgtgtttaaagtttaggtcgagtaaagcgcaaatct

SEQ ID NO: 8 - DNA, 7582 bp, synthetic construct
  source 1..7582: mol_type = other DNA
gtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctcgaaattaaccctcactaaagggaacaaaagctggtacctcgcgcgacttggtttgccattctttagcgcgcgtcgcgtcacacagcttggccacaatgtggtttttgtcaaacgaagattctatgacgtgtttaaagtttaggtcgagtaaagcgcaaatcttttttaaccctagaaagatagtctgcgtaaaattgacgcatgcattcttgaaatattgctctctctttctaaatagcgcgaatccgtcgctgtgcatttaggacatctcagtcgccgcttggagctcccgtgaggcgtgcttgtcaatgcggtaagtgtcactgattttgaactataacgaccgcgtgagtcaaaatgacgcatgattatcttttacgtgacttttaagatttaactcatacgataattatattgttatttcatgttctacttacgtgataacttattatatatatattttcttgttatagatatcatcaactttgtatagaaaagttgctcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattgcaagtttgtacaaaaaagcaggctgaatgccgccaccatggctaccgagtataaaccgacggtccgacttgcgactcgggatgacgttccacgcgccgtgaggactctggccgccgccttcgctgactacccggccacacggcacacagttgaccccgacagacacatagagcgggtcacagagttgcaagaacttttcctgacgcgggttggactcgatattggaaaagtttgggttgccgacgatggtgccgcagttgcggtgtggactactccagaaagtgtagaagctggagccgtattcgctgaaatagggccacggatggcagagctgtctggtagcaggctcgcagcgcagcagcagatggaaggccttctcgctccgcatcggcctaaggaaccggcgtggttcctggctacggtgggagtctcacctgaccatcaaggaaagggattgggaagtgctgtcgttcttccaggcgtggaagcggccgaacgagcaggcgtcccagccttcttggaaacgagcgcacccaggaatcttccattctacgagcgactgggtttcactgtgacagcagacgtagaagtccctgaagggcctcggacttggtgcatgaccagaaagcctggggcttgaaacgccatgatccagacatgataagatacattgatgagtttggacaaaccacaactagaatgcagtgaggcgcgcctggatacttctgcctgcaggggagatgtcttccaaattgacggctagctcagtcctaggtacagtgctagcataccagataagttcgtcggtcggttatgcagtttaaggtttacacctataaaagagagagccgttatcgtctgtttgtggatgtacagagtgatattattgacacgcccgggcgacggatggtgatccccctggccagtgcacgtctgctgtcagataaagtctcccgtgaactttacccggtggtgcatatcggggatgaaagctggcgcatgatgaccaccgatatggccagtgtgccggtgtccgttatcggggaagaagtggctgatctcagccaccgcgaaaatgacatcaaaaacgccattaacctgatgttctggggaatataataatggcctcggtaccaaattccagaaaagaggcctcccgaaaggggggccttttttcgttttggtccgtggcggaccaaaacgaaaaaagacgcttttcagcgtcttattgttcgtctttggtaccgaggccattatttgtatagttcatccatgccaccggtagaatgacgaccctccgcgcgctcatattgctctacgatcgtataatcttcattatgagataagatgtccagtttaatatccacattgtacgcgccaggcagctgcacaggtttcttggctttgtacgtggttttcacttcagcgtcataatggccgccatctttcagtttcaggcgctgtttaatttcgcctttcagcgcaccatcttccggatacatgcgttcggtagacgcctcccaacccatcgtcctacactgcatcaccgggccatcagatggaaaattagtaccacgcagtttaactttatagatgaactcgccatcctgaagggaggagtcctgagtgacggtcacgacaccaccatcttcaaaattcattacgcgttcccagttgaaaccttccggaaaagacagcttcagatagtccgggatatccgctgggtgtttaacatacgctttagaaccgtacataaattgcgggctcaggatgtcccacgcaaaaggcagcgggccgcctttagtcactttcagtttggcggtctgggtgccttcatacggacggccctcgccttcgccttcgatctcgaactcgtgaccgttaacagaaccctccatgtgaactttgaagcgcatgaactctttaatgatagctaagttgttttcttctcctttactcaccatttagtatttctcctctttcactaggtcgagtagctagcataatacctaggactgagctagctgtaaagaaggaagacattcgcgcggccgcgcctgggcttacttaattaacgagagcataatattgatatgtgccaaagttgtttctgactgactaataagtataatttgtttctattatgtataggttaagctaattacttattttataatacaacatgactgtttttaaagtacaaaataagtttatttttgtaaaagagagaatgtttaaaagttttgttactttatagaagaaattttgagtttttgtttttttttaataaataaataaacataaataaattgtttgttgaatttattattagtatgtaagtgtaaatataataaaacttaatatctattcaaattaataaataaacctcgatatacagaccgataaaacacatgcgtcaattttacgcatgattatctttaacgtacgtcacaatatgattatctttctagggttaaataatagtttctaatttttttattattcagcctgctgtcgtgaataccgagctccaattcgccctatagtgagtcgtattacaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattg

SEQ ID NO: 9 - DNA, 7582 bp, synthetic construct
  source 1..7582: mol_type = other DNA
cgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctcgaaattaaccctcactaaagggaacaaaagctggtacctcgcgcgacttggtttgccattctttagcgcgcgtcgcgtcacacagcttggccacaatgtggtttttgtcaaacgaagattctatgacgtgtttaaagtttaggtcgagtaaagcgcaaatcttttttaaccctagaaagatagtctgcgtaaaattgacgcatgcattcttgaaatattgctctctctttctaaatagcgcgaatccgtcgctgtgcatttaggacatctcagtcgccgcttggagctcccgtgaggcgtgcttgtcaatgcggtaagtgtcactgattttgaactataacgaccgcgtgagtcaaaatgacgcatgattatcttttacgtgacttttaagatttaactcatacgataattatattgttatttcatgttctacttacgtgataacttattatatatatattttcttgttatagatatcatcaactttgtatagaaaagttgctcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattgcaagtttgtacaaaaaagcaggctgaatgccgccaccatggctaccgagtataaaccgacggtccgacttgcgactcgggatgacgttccacgcgccgtgaggactctggccgccgccttcgctgactacccggccacacggcacacagttgaccccgacagacacatagagcgggtcacagagttgcaagaacttttcctgacgcgggttggactcgatattggaaaagtttgggttgccgacgatggtgccgcagttgcggtgtggactactccagaaagtgtagaagctggagccgtattcgctgaaatagggccacggatggcagagctgtctggtagcaggctcgcagcgcagcagcagatggaaggccttctcgctccgcatcggcctaaggaaccggcgtggttcctggctacggtgggagtctcacctgaccatcaaggaaagggattgggaagtgctgtcgttcttccaggcgtggaagcggccgaacgagcaggcgtcccagccttcttggaaacgagcgcacccaggaatcttccattctacgagcgactgggtttcactgtgacagcagacgtagaagtccctgaagggcctcggacttggtgcatgaccagaaagcctggggcttgaaacgccatgatccagacatgataagatacattgatgagtttggacaaaccacaactagaatgcagtgaggcgcgcctggatacttctgcctgcaggggagatgtcttccaaattgacggctagctcagtcctaggtacagtgctagcataccagataagttcgtcggtcggttatgcagtttaaggtttacacctataaaagagagagccgttatcgtctgtttgtggatgtacagagtgatattattgacacgcccgggcgacggatggtgatccccctggccagtgcacgtctgctgtcagataaagtctcccgtgaactttacccggtggtgcatatcggggatgaaagctggcgcatgatgaccaccgatatggccagtgtgccggtgtccgttatcggggaagaagtggctgatctcagccaccgcgaaaatgacatcaaaaacgccattaacctgatgttctggggaatataataatggcctcggtaccaaattccagaaaagaggcctcccgaaaggggggccttttttcgttttggtccgtggcggaccaaaacgaaaaaagacgcttttcagcgtcttattgttcgtctttggtaccgaggccattatttgtatagttcatccatgccaccggtagaatgacgaccctccgcgcgctcatattgctctacgatcgtataatcttcattatgagataagatgtccagtttaatatccacattgtacgcgccaggcagctgcacaggtttcttggctttgtacgtggttttcacttcagcgtcataatggccgccatctttcagtttcaggcgctgtttaatttcgcctttcagcgcaccatcttccggatacatgcgttcggtagacgcctcccaacccatcgtcctacactgcatcaccgggccatcagatggaaaattagtaccacgcagtttaactttatagatgaactcgccatcctgaagggaggagtcctgagtgacggtcacgacaccaccatcttcaaaattcattacgcgttcccagttgaaaccttccggaaaagacagcttcagatagtccgggatatccgctgggtgtttaacatacgctttagaaccgtacataaattgcgggctcaggatgtcccacgcaaaaggcagcgggccgcctttagtcactttcagtttggcggtctgggtgccttcatacggacggccctcgccttcgccttcgatctcgaactcgtgaccgttaacagaaccctccatgtgaactttgaagcgcatgaactctttaatgatagctaagttgttttcttctcctttactcaccatttagtatttctcctctttcactaggtcgagtagctagcataatacctaggactgagctagctgtaaagaaggaagacatcagtgcggccgcgcctgggcttacttaattaacgagagcataatattgatatgtgccaaagttgtttctgactgactaataagtataatttgtttctattatgtataggttaagctaattacttattttataatacaacatgactgtttttaaagtacaaaataagtttatttttgtaaaagagagaatgtttaaaagttttgttactttatagaagaaattttgagtttttgtttttttttaataaataaataaacataaataaattgtttgttgaatttattattagtatgtaagtgtaaatataataaaacttaatatctattcaaattaataaataaacctcgatatacagaccgataaaacacatgcgtcaattttacgcatgattatctttaacgtacgtcacaatatgattatctttctagggttaaataatagtttctaatttttttattattcagcctgctgtcgtgaataccgagctccaattcgccctatagtgagtcgtattacaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgc

SEQ ID NO: 10 - DNA, 7582 bp, synthetic construct
  source 1..7582: mol_type = other DNA
ctttacactttatgcttccggctcgtatgttgtgtggaattgtgagcggataacaatttcacacaggaaacagctatgaccatgattacgccaagctcgaaattaaccctcactaaagggaacaaaagctggtacctcgcgcgacttggtttgccattctttagcgcgcgtcgcgtcacacagcttggccacaatgtggtttttgtcaaacgaagattctatgacgtgtttaaagtttaggtcgagtaaagcgcaaatcttttttaaccctagaaagatagtctgcgtaaaattgacgcatgcattcttgaaatattgctctctctttctaaatagcgcgaatccgtcgctgtgcatttaggacatctcagtcgccgcttggagctcccgtgaggcgtgcttgtcaatgcggtaagtgtcactgattttgaactataacgaccgcgtgagtcaaaatgacgcatgattatcttttacgtgacttttaagatttaactcatacgataattatattgttatttcatgttctacttacgtgataacttattatatatatattttcttgttatagatatcatcaactttgtatagaaaagttgctcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattgcaagtttgtacaaaaaagcaggctgaatgccgccaccatggctaccgagtataaaccgacggtccgacttgcgactcgggatgacgttccacgcgccgtgaggactctggccgccgccttcgctgactacccggccacacggcacacagttgaccccgacagacacatagagcgggtcacagagttgcaagaacttttcctgacgcgggttggactcgatattggaaaagtttgggttgccgacgatggtgccgcagttgcggtgtggactactccagaaagtgtagaagctggagccgtattcgctgaaatagggccacggatggcagagctgtctggtagcaggctcgcagcgcagcagcagatggaaggccttctcgctccgcatcggcctaaggaaccggcgtggttcctggctacggtgggagtctcacctgaccatcaaggaaagggattgggaagtgctgtcgttcttccaggcgtggaagcggccgaacgagcaggcgtcccagccttcttggaaacgagcgcacccaggaatcttccattctacgagcgactgggtttcactgtgacagcagacgtagaagtccctgaagggcctcggacttggtgcatgaccagaaagcctggggcttgaaacgccatgatccagacatgataagatacattgatgagtttggacaaaccacaactagaatgcagtgaggcgcgcctggatacttctgcctgcaggggagatgtcttccaaattgacggctagctcagtcctaggtacagtgctagcataccagataagttcgtcggtcggttatgcagtttaaggtttacacctataaaagagagagccgttatcgtctgtttgtggatgtacagagtgatattattgacacgcccgggcgacggatggtgatccccctggccagtgcacgtctgctgtcagataaagtctcccgtgaactttacccggtggtgcatatcggggatgaaagctggcgcatgatgaccaccgatatggccagtgtgccggtgtccgttatcggggaagaagtggctgatctcagccaccgcgaaaatgacatcaaaaacgccattaacctgatgttctggggaatataataatggcctcggtaccaaattccagaaaagaggcctcccgaaaggggggccttttttcgttttggtccgtggcggaccaaaacgaaaaaagacgcttttcagcgtcttattgttcgtctttggtaccgaggccattatttgtatagttcatccatgccaccggtagaatgacgaccctccgcgcgctcatattgctctacgatcgtataatcttcattatgagataagatgtccagtttaatatccacattgtacgcgccaggcagctgcacaggtttcttggctttgtacgtggttttcacttcagcgtcataatggccgccatctttcagtttcaggcgctgtttaatttcgcctttcagcgcaccatcttccggatacatgcgttcggtagacgcctcccaacccatcgtcctacactgcatcaccgggccatcagatggaaaattagtaccacgcagtttaactttatagatgaactcgccatcctgaagggaggagtcctgagtgacggtcacgacaccaccatcttcaaaattcattacgcgttcccagttgaaaccttccggaaaagacagcttcagatagtccgggatatccgctgggtgtttaacatacgctttagaaccgtacataaattgcgggctcaggatgtcccacgcaaaaggcagcgggccgcctttagtcactttcagtttggcggtctgggtgccttcatacggacggccctcgccttcgccttcgatctcgaactcgtgaccgttaacagaaccctccatgtgaactttgaagcgcatgaactctttaatgatagctaagttgttttcttctcctttactcaccatttagtatttctcctctttcactaggtcgagtagctagcataatacctaggactgagctagctgtaaagaaggaagacattagggcggccgcgcctgggcttacttaattaacgagagcataatattgatatgtgccaaagttgtttctgactgactaataagtataatttgtttctattatgtataggttaagctaattacttattttataatacaacatgactgtttttaaagtacaaaataagtttatttttgtaaaagagagaatgtttaaaagttttgttactttatagaagaaattttgagtttttgtttttttttaataaataaataaacataaataaattgtttgttgaatttattattagtatgtaagtgtaaatataataaaacttaatatctattcaaattaataaataaacctcgatatacagaccgataaaacacatgcgtcaattttacgcatgattatctttaacgtacgtcacaatatgattatctttctagggttaaataatagtttctaatttttttattattcagcctgctgtcgtgaataccgagctccaattcgccctatagtgagtcgtattacaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccagg

SEQ ID NO: 11 - DNA, 3510 bp, synthetic construct
  source 1..3510: mol_type = other DNA
ctgctccctgcttgtgtgttggaggtcgctgagtaggatcctgaagacatggagtcgctgagacgcaaattgacggctagctcagtcctaggtacagtgctagcataccagataagttcgtcggtcggttatgcagtttaaggtttacacctataaaagagagagccgttatcgtctgtttgtggatgtacagagtgatattattgacacgcccgggcgacggatggtgatccccctggccagtgcacgtctgctgtcagataaagtctcccgtgaactttacccggtggtgcatatcggggatgaaagctggcgcatgatgaacaccgatatggccagtgtgccggtgtccgttatcggggaagaagtggctgatctcagccaccgcgaaaatgacatcaaaaacgccattaacctgatgttctggggaatataataatggcctcggtaccaaattccagaaaagaggcctcccgaaaggggggccttttttcgttttggtccgtggcggaccaaaacgaaaaaagacgcttttcagcgtcttattgttcgtctttggtaccgaggccattatttgtatagttcatccatgccaccggtagaatgacgaccctccgcgcgctcatattgctctacgatcgtataatcttcattatgagataagatgtccagtttaatatccacattgtacgcgccaggcagctgcacaggtttcttggctttgtacgtggttttcacttcagcgtcataatggccgccatctttcagtttcaggcgctgtttaatttcgcctttcagcgcaccatcttccggatacatgcgttcggtagacgcctcccaacccatcgtcctacactgcatcaccgggccatcagatggaaaattagtaccacgcagtttaactttatagatgaactcgccatcctgaagggaggagtcctgagtgacggtcacgacaccaccatcttcaaaattcattacgcgttcccagttgaaaccttccggaaaagacagcttcagatagtccgggatatccgctgggtgtttaacatacgctttagaaccgtacataaattgcgggctcaggatgtcccacgcaaaaggcagcgggccgcctttagtcactttcagtttggcggtctgggtgccttcatacggacggccctcgccttcgccttcgatctcgaactcgtgaccgttaacagaaccctccatgtgaactttgaagcgcatgaactctttaatgatagctaagttgttttcttctcctttactcaccatttagtatttctcctctttcactaggtcgagtagctagcataatacctaggactgagctagctgtaaagaagcctgcagggctagcaacgtagcgtctcacagtggcaatgtcttccggatccgggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgcacatccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttacttgatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgatgtggtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagccagtcgaaatacgcgatcgctgttaaaaggacaattacaaacaggaatcgaatgcaaccggcgcaggaacactgccagcgcatcaacaatattttcacctgaatcaggatattcttctaatacctggaatgctgtttttccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaagcgatagattgtcgcacctgattgcccgacattatcgcgagcccatttatacccatataaatcagcatccatgttggaatttaatcgcggcctcgacgtttcccgttgaatatggctcataacaccccttgtattactgtttatgtaagcagacagttttattgttcatgatgatatatttttatcttgtgcaatgtaacatcagagattttgagacacggaatcgacgctcaagtcagaggtcctgacggactcggatcgggagatctcccgatcccctatggtgcactctcagtacaatctgctctgatgccgcatagttaagccagtat

SEQ ID NO: 12 - DNA, 7467 bp, synthetic construct
  source 1..7467: mol_type = other DNA
caactttgtatagaaaagttgctcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattgcaagtttgtacaaaaaagcaggctgccaccatgggcagcagcctggacgacgagcacatcctgagcgccctgctgcagagcgacgacgagctggtcggcgaggacagcgacagcgaggtgagcgaccacgtgagcgaggacgacgtgcagtccgacaccgaggaggccttcatcgacgaggtgcacgaggtgcagcctaccagcagcggctccgagatcctggacgagcagaacgtgatcgagcagcccggcagctccctggccagcaacaggatcctgaccctgccccagaggaccatcaggggcaagaacaagcactgctggtccacctccaagcccaccaggcggagcagggtgtccgccctgaacatcgtgagaagccagaggggccccaccaggatgtgcaggaacatctacgaccccctgctgtgcttcaagctgttcttcaccgacgagatcatcagcgagatcgtgaagtggaccaacgccgagatcagcctgaagaggcgggagagcatgacctccgccaccttcagggacaccaacgaggacgagatctacgccttcttcggcatcctggtgatgaccgccgtgaggaaggacaaccacatgagcaccgacgacctgttcgacagatccctgagcatggtgtacgtgagcgtgatgagcagggacagattcgacttcctgatcagatgcctgaggatggacgacaagagcatcaggcccaccctgcgggagaacgacgtgttcacccccgtgagaaagatctgggacctgttcatccaccagtgcatccagaactacacccctggcgcccacctgaccatcgacgagcagctgctgggcttcaggggcaggtgccccttcagggtctatatccccaacaagcccagcaagtacggcatcaagatcctgatgatgtgcgacagcggcaccaagtacatgatcaacggcatgccctacctgggcaggggcacccagaccaacggcgtgcccctgggcgagtactacgtgaaggagctgtccaagcccgtccacggcagctgcagaaacatcacctgcgacaactggttcaccagcatccccctggccaagaacctgctgcaggagccctacaagctgaccatcgtgggcaccgtgagaagcaacaagagagagatccccgaggtcctgaagaacagcaggtccaggcccgtgggcaccagcatgttctgcttcgacggccccctgaccctggtgtcctacaagcccaagcccgccaagatggtgtacctgctgtccagctgcgacgaggacgccagcatcaacgagagcaccggcaagccccagatggtgatgtactacaaccagaccaagggcggcgtggacaccctggaccagatgtgcagcgtgatgacctgcagcagaaagaccaacaggtggcccatggccctgctgtacggcatgatcaacatcgcctgcatcaacagcttcatcatctacagccacaacgtgagcagcaagggcgagaaggtgcagagccggaaaaagttcatgcggaacctgtacatgggcctgacctccagcttcatgaggaagaggctggaggcccccaccctgaagagatacctgagggacaacatcagcaacatcctgcccaaagaggtgcccggcaccagcgacgacagcaccgaggagcccgtgatgaagaagaggacctactgcacctactgtcccagcaagatcagaagaaaggccagcgccagctgcaagaagtgtaagaaggtcatctgccgggagcacaacatcgacatgtgccagagctgtttctgaacccagctttcttgtacaaagtggtgatggccggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcgccaccatggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagtaactcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgggcggccgcggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcggcgcgccgcggccgc

SEQ ID NO: 13 - DNA, 4420 bp, synthetic construct
  source 1..4420: mol_type = other DNA
atgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagccagtcgaaatacgcgatcgctgttaaaaggacaattacaaacaggaatcgaatgcaaccggcgcaggaacactgccagcgcatcaacaatattttcacctgaatcaggatattcttctaatacctggaatgctgtttttccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaagcgatagattgtcgcacctgattgcccgacattatcgcgagcccatttatacccatataaatcagcatccatgttggaatttaatcgcggcctcgacgtttcccgttgaatatggctcataacaccccttgtattactgtttatgtaagcagacagttttattgttcatgatgatattattttatcttgtgcaatgtaacatcagagattttgagacacggaatcgacgctcaagtcagaggtcctgacgtcgacggatcgggagatctcccgatcccctatggtgcactctcagtacaatctgctctgatgccgcatagttaagccagtatctgctccctgcttgtgtgttggaggtcgctgagtaggatcctgaagacatggagtcgctcttttgcaatgctctttgggaaattatattagcctaattactaatttcctggccctcaaggtgacttgcttgaacttgccaccttcgccactagggggcagcattggtttacacagggtgaacaaacgttcaattctaaactaatattctttggtgggaaagtgtgttcattttggtttgttttgaagaagtcacttcatacctttgaactggggttaagctggttaactcccaaaattccaccagttccaatatcctatggaaaaaccccaaaaccactcaaaccagatatactcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagaaccaagcgaatgccgccaccatggctatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaaacgccatgaattcactcctcaggtgcaggctgcctatcagaaggtggtggctggtgtggccaatgccctggctcacaaataccactgagatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgcccatatgctggctgccatgaacaaaggttggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatggagatcagtggcaatgtcttccggatccgggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtttttttgtttgcaagcagcagattacgcgcagaaaaaaaggatctcaagaagatcctttgatcttttctacggggtctgacgctcagtggaacgaatgtggtaatgctctgccagtgttacaaccaattaaccaattctgattagaaaaactcatcgagcatcaa

SEQ ID NO: 14 - DNA, 4417 bp, synthetic construct
  source 1..4417: mol_type = other DNA
cgtttcccgttgaatatggctcataacaccccttgtattactgtttatgtaagcagacagttttattgttcatgatgatattattttatcttgtgcaatgtaacatcagagattttgagacacggaatcgacgctcaagtcagaggtcctgacgtcgacggatcgggagatctcccgatcccctatggtgcactctcagtacaatctgctctgatgccgcatagttaagccagtatctgctccctgcttgtgtgttggaggtcgctgagtaggatcctgaagacatggagtcgctcttttgcaatgctctttgggaaattatattagcctaattactaatttcctggccctcaaggtgacttgcttgaacttgccaccttcgccactagggggcagcattggtttacacagggtgaacaaacgttcaattctaaactaatattctttggtgggaaagtgtgttcattttggtttgttttgaagaagtcacttcatacctttgaactggggttaagctggttaactcccaaaattccaccagttccaatatcctatggaaaaaccccaaaaccactcaaaccagatatactcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagaaccaagcgaatgccgccaccatggctggcaatagcgttagcaaaggtgaggaagataacatggcgattataaaggaatttatgcggttcaaggtgcacatggagggttcagttaatggacacgagttcgaaatcgaaggtgagggggaaggtagaccttacgaaggaactcagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccttgcaggacggcgagttcatctacaaggtgaagctgcgcggcaccaacttcccctccgacggccccgtaatgcagaagaaaaccatgggctggcaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtataagtaaaacgccatgaattcactcctcaggtgcaggctgcctatcagaaggtggtggctggtgtggccaatgccctggctcacaaataccactgagatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgcccatatgctggctgccatgaacaaaggttggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatggagatcagtggcaatgtcttccggatccgggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtttttttgtttgcaagcagcagattacgcgcagaaaaaaaggatctcaagaagatcctttgatcttttctacggggtctgacgctcagtggaacgaatgtggtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagccagtcgaaatacgcgatcgctgttaaaaggacaattacaaacaggaatcgaatgcaaccggcgcaggaacactgccagcgcatcaacaatattttcacctgaatcaggatattcttctaatacctggaatgctgtttttccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaagcgatagattgtcgcacctgattgcccgacattatcgcgagcccatttatacccatataaatcagcatccatgttggaatttaatcgcggcctcga

SEQ ID NO: 15 - DNA, 4652 bp, synthetic construct
  source 1..4652: mol_type = other DNA
atgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagccagtcgaaatacgcgatcgctgttaaaaggacaattacaaacaggaatcgaatgcaaccggcgcaggaacactgccagcgcatcaacaatattttcacctgaatcaggatattcttctaatacctggaatgctgtttttccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaagcgatagattgtcgcacctgattgcccgacattatcgcgagcccatttatacccatataaatcagcatccatgttggaatttaatcgcggcctcgacgtttcccgttgaatatggctcataacaccccttgtattactgtttatgtaagcagacagttttattgttcatgatgatattattttatcttgtgcaatgtaacatcagagattttgagacacggaatcgacgctcaagtcagaggtcctgacgtcgacggatcgggagatctcccgatcccctatggtgcactctcagtacaatctgctctgatgccgcatagttaagccagtatctgctccctgcttgtgtgttggaggtcgctgagtaggatcctgaagacatggagtcgctcttttgcaatgctctttgggaaattatattagcctaattactaatttcctggccctcaaggtgacttgcttgaacttgccaccttcgccactagggggcagcattggtttacacagggtgaacaaacgttcaattctaaactaatattctttggtgggaaagtgtgttcattttggtttgttttgaagaagtcacttcatacctttgaactggggttaagctggttaactcccaaaattccaccagttccaatatcctatggaaaaaccccaaaaccactcaaaccagatatactcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagaaccaagcgaatgccgccaccatggctgctgaaggttccgtcgccaggcagcctgacctcttgacctgcgacgatgagccgatccatatccccggtgccatccaaccgcatggactgctgctcgccctcgccgccgacatgacgatcgttgccggcagcgacaaccttcccgaactcaccggactggcgatcggcgccctgatcggccgctctgcggccgatgtgttcgactcggaaacgcacaaccgtctgacgatcgccttggccgagcccggggcggccgtcggagcaccgatcactgtcggcttcacgatgcgaaaggacgcaggcttcatcggctcctggcatcgccatgatcagctcatcttcctggagctggagcctccccagcgggacgtcgccgagccgcaggcgttcttccgccgcaccaacagcgccatccgccgcctgcaggccgccgaaaccttggaaagcgcctgcgccgccgcggcgcaagaggtgcggaagattaccggcttcgatcgggtgatgatctatcgcttcgcctccgacttcagcggcgaagtgatcgcagaggatcggtgcgccgaggtcgagtcaaaactaggcctgcactatcctgcctcaaccgtgccggcgcaggcccgtcggctctataccatcaacccggtacggatcattcccgatatcaattatcggccggtgccggtcaccccagacctcaatccggtcaccgggcggccgattgatcttagcttcgccatcctgcgcagcgtgtcgcccgtccatctggagttcatgcgcaacataggcatgcacggcacgatgtcgatctcgattttgcgcggcgagcgactgtggggattgatcgtttgccatcaccgaacgccgtactacgtcgatctcgatggccgccaagcctgcgagctagtcgcccaggttctggcctggcagatcggcgtgatggaagagtaatagaacgccatgaattcactcctcaggtgcaggctgcctatcagaaggtggtggctggtgtggccaatgccctggctcacaaataccactgagatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgcccatatgctggctgccatgaacaaaggttggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatggagatccagtggcaatgtcttccggatccgggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtttttttgtttgcaagcagcagattacgcgcagaaaaaaaggatctcaagaagatcctttgatcttttctacggggtctgacgctcagtggaacgaatgtggtaatgctctgccagtgttacaaccaattaaccaattctgattagaaaaactcatcgagcatcaa

SEQ ID NO: 16 - DNA, 4412 bp, synthetic construct
  source 1..4412: mol_type = other DNA
tttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagccagtcgaaatacgcgatcgctgttaaaaggacaattacaaacaggaatcgaatgcaaccggcgcaggaacactgccagcgcatcaacaatattttcacctgaatcaggatattcttctaatacctggaatgctgtttttccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaagcgatagattgtcgcacctgattgcccgacattatcgcgagcccatttatacccatataaatcagcatccatgttggaatttaatcgcggcctcgacgtttcccgttgaatatggctcataacaccccttgtattactgtttatgtaagcagacagttttattgttcatgatgatattattttatcttgtgcaatgtaacatcagagattttgagacacggaatcgacgctcaagtcagaggtcctgacgtcgacggatcgggagatctcccgatcccctatggtgcactctcagtacaatctgctctgatgccgcatagttaagccagtatctgctccctgcttgtgtgttggaggtcgctgagtaggatcctgaagacatggagtcgctcttttgcaatgctctttgggaaattatattagcctaattactaatttcctggccctcaaggtgacttgcttgaacttgccaccttcgccactagggggcagcattggtttacacagggtgaacaaacgttcaattctaaactaatattctttggtgggaaagtgtgttcattttggtttgttttgaagaagtcacttcatacctttgaactggggttaagctggttaactcccaaaattccaccagttccaatatcctatggaaaaaccccaaaaccactcaaaccagatatactcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagaaccaagcgaatgccgccaccatggctgtcagtaaaggggaagagcttataaaggaaaatatgcacatgaagctctacatggagggcactgtagataaccaccatttcaaatgtacctctgaaggggagggcaagccatacgaaggtactcaaaccatgcgaataaaagtagttgaaggcgggcctcttccctttgcattcgacattctcgcaacctcttttctgtacggcagtaagactttcataaaccacactcaaggcattccagacttcttcaagcaatcattccccgaaggattcacctgggagcgagttactacttatgaggacggaggagtccttactgcaacccaagacacctcactgcaagatgggtgcctgatttacaatgtaaagatcagaggggtgaatttcacaagcaatgggccagttatgcaaaaaaagacccttggatgggaggccttcaccgagacactgtacccagccgatggtggactggagggcaggaatgacatggccctcaagctcgtcggaggcagtcacttgattgccaacgccaaaaccacttaccgctctaagaaacctgctaaaaacctgaagatgcccggcgtctattatgtggactatcgacttgagagaattaaggaggcaaacaacgagacttatgtcgaacagcatgaagttgccgtggctaggtactgtgatttgcccagcaaattgggtcataaacttaactgaaacgccatgaattcactcctcaggtgcaggctgcctatcagaaggtggtggctggtgtggccaatgccctggctcacaaataccactgagatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgcccatatgctggctgccatgaacaaaggttggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatggagatccagtggcaatgtcttccggatccgggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtttttttgtttgcaagcagcagattacgcgcagaaaaaaaggatctcaagaagatcctttgatcttttctacggggtctgacgctcagtggaacgaatgtggtaatgctctgccagtgttacaaccaattaaccaattctgattagaaaaactcatcgagcatcaaatgaaactgcaatttattcatatcaggattatcaataccata

SEQ ID NO: 17 - DNA, 4362 bp, synthetic construct
  source 1..4362: mol_type = other DNA
cgtttcccgttgaatatggctcataacaccccttgtattactgtttatgtaagcagacagttttattgttcatgatgatattattttatcttgtgcaatgtaacatcagagattttgagacacggaatcgacgctcaagtcagaggtcctgacgtcgacggatcgggagatctcccgatcccctatggtgcactctcagtacaatctgctctgatgccgcatagttaagccagtatctgctccctgcttgtgtgttggaggtcgctgagtaggatcctgaagacatggagtcgctcttttgcaatgctctttgggaaattatattagcctaattactaatttcctggccctcaaggtgacttgcttgaacttgccaccttcgccactagggggcagcattggtttacacagggtgaacaaacgttcaattctaaactaatattctttggtgggaaagtgtgttcattttggtttgttttgaagaagtcacttcatacctttgaactggggttaagctggttaactcccaaaattccaccagttccaatatcctatggaaaaaccccaaaaccactcaaaccagatatactcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagaaccaagcgaatgccgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaaacgccatgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagagaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagtcggggtttcaccatattggccaggctggtgtccaactcctaatctcaggtgatctacccaccttggcctcccaaattgctgggattacaggcgtgaaccactgctcccttccctgtccttcagtggcaatgtcttccggatccgggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtttttttgtttgcaagcagcagattacgcgcagaaaaaaaggatctcaagaagatcctttgatcttttctacggggtctgacgctcagtggaacgaatgtggtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagccagtcgaaatacgcgatcgctgttaaaaggacaattacaaacaggaatcgaatgcaaccggcgcaggaacactgccagcgcatcaacaatattttcacctgaatcaggatattcttctaatacctggaatgctgtttttccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaagcgatagattgtcgcacctgattgcccgacattatcgcgagcccatttatacccatataaatcagcatccatgttggaatttaatcgcggcctcga

SEQ ID NO: 18 - DNA, 4233 bp, synthetic construct
  source 1..4233: mol_type = other DNA
cgtttcccgttgaatatggctcataacaccccttgtattactgtttatgtaagcagacagttttattgttcatgatgatattattttatcttgtgcaatgtaacatcagagattttgagacacggaatcgacgctcaagtcagaggtcctgacgtcgacggatcgggagatctcccgatcccctatggtgcactctcagtacaatctgctctgatgccgcatagttaagccagtatctgctccctgcttgtgtgttggaggtcgctgagtaggatcctgaagacatggagtcgctcttttgcaatgctctttgggaaattatattagcctaattactaatttcctggccctcaaggtgacttgcttgaacttgccaccttcgccactagggggcagcattggtttacacagggtgaacaaacgttcaattctaaactaatattctttggtgggaaagtgtgttcattttggtttgttttgaagaagtcacttcatacctttgaactggggttaagctggttaactcccaaaattccaccagttccaatatcctatggaaaaaccccaaaaccactcaaaggatctgcgatcgctccggtgcccgtcagtgggcagagcgcacatcgcccacagtccccgagaagttggggggaggggtcggcaattgaacgggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagctgaagcttcgaggggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagtccgggcctttgtccggcgctcccttggagcctacctagactcagccggctctccacgctttgcctgaccctgcttgctcaactctacgtctttgtttcgttttctgttctgcgccgttacagatccaagctgtgaccggcgcctacgaatgccgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaaacgccatgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagagaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagtcggggtttcaccatattggccaggctggtgtccaactcctaatctcaggtgatctacccaccttggcctcccaaattgctgggattacaggcgtgaaccactgctcccttccctgtccttcagtggcaatgtcttccggatccgggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtttttttgtttgcaagcagcagattacgcgcagaaaaaaaggatctcaagaagatcctttgatcttttctacggggtctgacgctcagtggaacgaatgtggtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagccagtcgaaatacgcgatcgctgttaaaaggacaattacaaacaggaatcgaatgcaaccggcgcaggaacactgccagcgcatcaacaatattttcacctgaatcaggatattcttctaatacctggaatgctgtttttccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaagcgatagattgtcgcacctgattgcccgacattatcgcgagcccatttatacccatataaatcagcatccatgttggaatttaatcgcggcctcga

SEQ ID NO: 19 - DNA, 4152 bp, synthetic construct
  source 1..4152: mol_type = other DNA
cgtttcccgttgaatatggctcataacaccccttgtattactgtttatgtaagcagacagttttattgttcatgatgatattattttatcttgtgcaatgtaacatcagagattttgagacacggaatcgacgctcaagtcagaggtcctgacgtcgacggatcgggagatctcccgatcccctatggtgcactctcagtacaatctgctctgatgccgcatagttaagccagtatctgctccctgcttgtgtgttggaggtcgctgagtaggatcctgaagacatggagtcgctcttttgcaatgctctttgggaaattatattagcctaattactaatttcctggccctcaaggtgacttgcttgaacttgccaccttcgccactagggggcagcattggtttacacagggtgaacaaacgttcaattctaaactaatattctttggtgggaaagtgtgttcattttggtttgttttgaagaagtcacttcatacctttgaactggggttaagctggttaactcccaaaattccaccagttccaatatcctatggaaaaaccccaaaaccactcaaagaattctaccgggtaggggaggcgcttttcccaaggcagtctggagcatgcgctttagcagcaccgctgggcacttggcgctacacaagtggcctctggcctcgcacacattccacatccaccggtaggcgccaaccggctccgttctttggtggccccttcgcgccaccttctactcctcccctagtcaggaagttcccctccgccccgcagctcgcgtcgtgcaggacgtgacaaatggaagtagcacgtctaactagtctcgtgcagatggacagcaccgctgagcaatggaagcgggtaggcctttggggcagcggccaatagcagctttgctccttcgctttctgggctcaggggcggggcgggcgcccgaaggtcctccggaggcccggcattctgcacgcttcaaaagcgcacgtctgccgcgctgttctcctcttcctcatctccgggcctttcggaatgccgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaaacgccatgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagagaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagtcggggtttcaccatattggccaggctggtgtccaactcctaatctcaggtgatctacccaccttggcctcccaaattgctgggattacaggcgtgaaccactgctcccttccctgtccttcagtggcaatgtcttccggatccgggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtttttttgtttgcaagcagcagattacgcgcagaaaaaaaggatctcaagaagatcctttgatcttttctacggggtctgacgctcagtggaacgaatgtggtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagccagtcgaaatacgcgatcgctgttaaaaggacaattacaaacaggaatcgaatgcaaccggcgcaggaacactgccagcgcatcaacaatattttcacctgaatcaggatattcttctaatacctggaatgctgtttttccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaagcgatagattgtcgcacctgattgcccgacattatcgcgagcccatttatacccatataaatcagcatccatgttggaatttaatcgcggcctcga

SEQ ID NO: 20 - DNA, 8521 bp, synthetic construct
  source 1..8521: mol_type = other DNA
agcgcaaatcttttttaaccctagaaagatagtctgcgtaaaattgacgcatgcattcttgaaatattgctctctctttctaaatagcgcgaatccgtcgctgtgcatttaggacatctcagtcgccgcttggagctcccgtgaggcgtgcttgtcaatgcggtaagtgtcactgattttgaactataacgaccgcgtgagtcaaaatgacgcatgattatcttttacgtgacttttaagatttaactcatacgataattatattgttatttcatgttctacttacgtgataacttattatatatatattttcttgttatagatatcatcaactttgtatagaaaagttgctcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattgcaagtttgtacaaaaaagcaggctgaatgccgccaccatggctaccgagtataaaccgacggtccgacttgcgactcgggatgacgttccacgcgccgtgaggactctggccgccgccttcgctgactacccggccacacggcacacagttgaccccgacagacacatagagcgggtcacagagttgcaagaacttttcctgacgcgggttggactcgatattggaaaagtttgggttgccgacgatggtgccgcagttgcggtgtggactactccagaaagtgtagaagctggagccgtattcgctgaaatagggccacggatggcagagctgtctggtagcaggctcgcagcgcagcagcagatggaaggccttctcgctccgcatcggcctaaggaaccggcgtggttcctggctacggtgggagtctcacctgaccatcaaggaaagggattgggaagtgctgtcgttcttccaggcgtggaagcggccgaacgagcaggcgtcccagccttcttggaaacgagcgcacccaggaatcttccattctacgagcgactgggtttcactgtgacagcagacgtagaagtccctgaagggcctcggacttggtgcatgaccagaaagcctggggcttgaaacgccatgatccagacatgataagatacattgatgagtttggacaaaccacaactagaatgcagtgaggcgcgcctggatacttctgcctgcaggggagtcgctcttttgcaatgctctttgggaaattatattagcctaattactaatttcctggccctcaaggtgacttgcttgaacttgccaccttcgccactagggggcagcattggtttacacagggtgaacaaacgttcaattctaaactaatattctttggtgggaaagtgtgttcattttggtttgttttgaagaagtcacttcatacctttgaactggggttaagctggttaactcccaaaattccaccagttccaatatcctatggaaaaaccccaaaaccactcaaaccagatatactcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagaaccaagcgaatgccgccaccatggctatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaaacgccatgaattcactcctcaggtgcaggctgcctatcagaaggtggtggctggtgtggccaatgccctggctcacaaataccactgagatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgcccatatgctggctgccatgaacaaaggttggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatggagatcagtggcagcggccgcgcctgggcttacttaattaacgagagcataatattgatatgtgccaaagttgtttctgactgactaataagtataatttgtttctattatgtataggttaagctaattacttattttataatacaacatgactgtttttaaagtacaaaataagtttatttttgtaaaagagagaatgtttaaaagttttgttactttatagaagaaattttgagtttttgtttttttttaataaataaataaacataaataaattgtttgttgaatttattattagtatgtaagtgtaaatataataaaacttaatatctattcaaattaataaataaacctcgatatacagaccgataaaacacatgcgtcaattttacgcatgattatctttaacgtacgtcacaatatgattatctttctagggttaaataatagtttctaatttttttattattcagcctgctgtcgtgaataccgagctccaattcgccctatagtgagtcgtattacaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctcgaaattaaccctcactaaagggaacaaaagctggtacctcgcgcgacttggtttgccattctttagcgcgcgtcgcgtcacacagcttggccacaatgtggtttttgtcaaacgaagattctatgacgtgtttaaagtttaggtcgagtaa

SEQ ID NO: 21 - DNA, 8513 bp, synthetic construct
  source 1..8513: mol_type = other DNA
caggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctcgaaattaaccctcactaaagggaacaaaagctggtacctcgcgcgacttggtttgccattctttagcgcgcgtcgcgtcacacagcttggccacaatgtggtttttgtcaaacgaagattctatgacgtgtttaaagtttaggtcgagtaaagcgcaaatcttttttaaccctagaaagatagtctgcgtaaaattgacgcatgcattcttgaaatattgctctctctttctaaatagcgcgaatccgtcgctgtgcatttaggacatctcagtcgccgcttggagctcccgtgaggcgtgcttgtcaatgcggtaagtgtcactgattttgaactataacgaccgcgtgagtcaaaatgacgcatgattatcttttacgtgacttttaagatttaactcatacgataattatattgttatttcatgttctacttacgtgataacttattatatatatattttcttgttatagatatcatcaactttgtatagaaaagttgctcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattgcaagtttgtacaaaaaagcaggctgaatgccgccaccatggctaccgagtataaaccgacggtccgacttgcgactcgggatgacgttccacgcgccgtgaggactctggccgccgccttcgctgactacccggccacacggcacacagttgaccccgacagacacatagagcgggtcacagagttgcaagaacttttcctgacgcgggttggactcgatattggaaaagtttgggttgccgacgatggtgccgcagttgcggtgtggactactccagaaagtgtagaagctggagccgtattcgctgaaatagggccacggatggcagagctgtctggtagcaggctcgcagcgcagcagcagatggaaggccttctcgctccgcatcggcctaaggaaccggcgtggttcctggctacggtgggagtctcacctgaccatcaaggaaagggattgggaagtgctgtcgttcttccaggcgtggaagcggccgaacgagcaggcgtcccagccttcttggaaacgagcgcacccaggaatcttccattctacgagcgactgggtttcactgtgacagcagacgtagaagtccctgaagggcctcggacttggtgcatgaccagaaagcctggggcttgaaacgccatgatccagacatgataagatacattgatgagtttggacaaaccacaactagaatgcagtgaggcgcgcctggatacttctgcctgcaggggagtcgctcttttgcaatgctctttgggaaattatattagcctaattactaatttcctggccctcaaggtgacttgcttgaacttgccaccttcgccactagggggcagcattggtttacacagggtgaacaaacgttcaattctaaactaatattctttggtgggaaagtgtgttcattttggtttgttttgaagaagtcacttcatacctttgaactggggttaagctggttaactcccaaaattccaccagttccaatatcctatggaaaaaccccaaaaccactcaaaccagatatactcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagaaccaagcgaatgccgccaccatggctgtcagtaaaggggaagagcttataaaggaaaatatgcacatgaagctctacatggagggcactgtagataaccaccatttcaaatgtacctctgaaggggagggcaagccatacgaaggtactcaaaccatgcgaataaaagtagttgaaggcgggcctcttccctttgcattcgacattctcgcaacctcttttctgtacggcagtaagactttcataaaccacactcaaggcattccagacttcttcaagcaatcattccccgaaggattcacctgggagcgagttactacttatgaggacggaggagtccttactgcaacccaagacacctcactgcaagatgggtgcctgatttacaatgtaaagatcagaggggtgaatttcacaagcaatgggccagttatgcaaaaaaagacccttggatgggaggccttcaccgagacactgtacccagccgatggtggactggagggcaggaatgacatggccctcaagctcgtcggaggcagtcacttgattgccaacgccaaaaccacttaccgctctaagaaacctgctaaaaacctgaagatgcccggcgtctattatgtggactatcgacttgagagaattaaggaggcaaacaacgagacttatgtcgaacagcatgaagttgccgtggctaggtactgtgatttgcccagcaaattgggtcataaacttaactgaaacgccatgaattcactcctcaggtgcaggctgcctatcagaaggtggtggctggtgtggccaatgccctggctcacaaataccactgagatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgcccatatgctggctgccatgaacaaaggttggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatggagatccagtggcagcggccgcgcctgggcttacttaattaacgagagcataatattgatatgtgccaaagttgtttctgactgactaataagtataatttgtttctattatgtataggttaagctaattacttattttataatacaacatgactgtttttaaagtacaaaataagtttatttttgtaaaagagagaatgtttaaaagttttgttactttatagaagaaattttgagtttttgtttttttttaataaataaataaacataaataaattgtttgttgaatttattattagtatgtaagtgtaaatataataaaacttaatatctattcaaattaataaataaacctcgatatacagaccgataaaacacatgcgtcaattttacgcatgattatctttaacgtacgtcacaatatgattatctttctagggttaaataatagtttctaatttttttattattcagcctgctgtcgtgaataccgagctccaattcgccctatagtgagtcgtattacaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttg

SEQ ID NO: 22 - DNA, 8749 bp, synthetic construct
  source 1..8749: mol_type = other DNA
ctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgcccatatgctggctgccatgaacaaaggttggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatggagatcagtggcagcggccgcgcctgggcttacttaattaacgagagcataatattgatatgtgccaaagttgtttctgactgactaataagtataatttgtttctattatgtataggttaagctaattacttattttataatacaacatgactgtttttaaagtacaaaataagtttatttttgtaaaagagagaatgtttaaaagttttgttactttatagaagaaattttgagtttttgtttttttttaataaataaataaacataaataaattgtttgttgaatttattattagtatgtaagtgtaaatataataaaacttaatatctattcaaattaataaataaacctcgatatacagaccgataaaacacatgcgtcaattttacgcatgattatctttaacgtacgtcacaatatgattatctttctagggttaaataatagtttctaatttttttattattcagcctgctgtcgtgaataccgagctccaattcgccctatagtgagtcgtattacaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctcgaaattaaccctcactaaagggaacaaaagctggtacctcgcgcgacttggtttgccattctttagcgcgcgtcgcgtcacacagcttggccacaatgtggtttttgtcaaacgaagattctatgacgtgtttaaagtttaggtcgagtaaagcgcaaatcttttttaaccctagaaagatagtctgcgtaaaattgacgcatgcattcttgaaatattgctctctctttctaaatagcgcgaatccgtcgctgtgcatttaggacatctcagtcgccgcttggagctcccgtgaggcgtgcttgtcaatgcggtaagtgtcactgattttgaactataacgaccgcgtgagtcaaaatgacgcatgattatcttttacgtgacttttaagatttaactcatacgataattatattgttatttcatgttctacttacgtgataacttattatatatatattttcttgttatagatatcatcaactttgtatagaaaagttgctcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattgcaagtttgtacaaaaaagcaggctgaatgccgccaccatggctaccgagtataaaccgacggtccgacttgcgactcgggatgacgttccacgcgccgtgaggactctggccgccgccttcgctgactacccggccacacggcacacagttgaccccgacagacacatagagcgggtcacagagttgcaagaacttttcctgacgcgggttggactcgatattggaaaagtttgggttgccgacgatggtgccgcagttgcggtgtggactactccagaaagtgtagaagctggagccgtattcgctgaaatagggccacggatggcagagctgtctggtagcaggctcgcagcgcagcagcagatggaaggccttctcgctccgcatcggcctaaggaaccggcgtggttcctggctacggtgggagtctcacctgaccatcaaggaaagggattgggaagtgctgtcgttcttccaggcgtggaagcggccgaacgagcaggcgtcccagccttcttggaaacgagcgcacccaggaatcttccattctacgagcgactgggtttcactgtgacagcagacgtagaagtccctgaagggcctcggacttggtgcatgaccagaaagcctggggcttgaaacgccatgatccagacatgataagatacattgatgagtttggacaaaccacaactagaatgcagtgaggcgcgcctggatacttctgcctgcaggggagtcgctcttttgcaatgctctttgggaaattatattagcctaattactaatttcctggccctcaaggtgacttgcttgaacttgccaccttcgccactagggggcagcattggtttacacagggtgaacaaacgttcaattctaaactaatattctttggtgggaaagtgtgttcattttggtttgttttgaagaagtcacttcatacctttgaactggggttaagctggttaactcccaaaattccaccagttccaatatcctatggaaaaaccccaaaaccactcaaaccagatatactcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagaaccaagcgaatgccgccaccatggctgctgaaggttccgtcgccaggcagcctgacctcttgacctgcgacgatgagccgatccatatccccggtgccatccaaccgcatggactgctgctcgccctcgccgccgacatgacgatcgttgccggcagcgacaaccttcccgaactcaccggactggcgatcggcgccctgatcggccgctctgcggccgatgtgttcgactcggaaacgcacaaccgtctgacgatcgccttggccgagcccggggcggccgtcggagcaccgatcactgtcggcttcacgatgcgaaaggacgcaggcttcatcggctcctggcatcgccatgatcagctcatcttcctggagctggagcctccccagcgggacgtcgccgagccgcaggcgttcttccgccgcaccaacagcgccatccgccgcctgcaggccgccgaaaccttggaaagcgcctgcgccgccgcggcgcaagaggtgcggaagattaccggcttcgatcgggtgatgatctatcgcttcgcctccgacttcagcggcgaagtgatcgcagaggatcggtgcgccgaggtcgagtcaaaactaggcctgcactatcctgcctcaaccgtgccggcgcaggcccgtcggctctataccatcaacccggtacggatcattcccgatatcaattatcggccggtgccggtcaccccagacctcaatccggtcaccgggcggccgattgatcttagcttcgccatcctgcgcagcgtgtcgcccgtccatctggagttcatgcgcaacataggcatgcacggcacgatgtcgatctcgattttgcgcggcgagcgactgtggggattgatcgtttgccatcaccgaacgccgtactacgtcgatctcgatggccgccaagcctgcgagctagtcgcccaggttctggcctggcagatcggcgtgatggaagagtaaaacgccatgaattcactcctcaggtgcaggctgcctatcagaaggtggtggctggtgtggccaatgccctggctcacaaataccactgagatctttttcc

SEQ ID NO: 23 - DNA, 10757 bp, synthetic construct
  source 1..10757: mol_type = other DNA
caggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctcgaaattaaccctcactaaagggaacaaaagctggtacctcgcgcgacttggtttgccattctttagcgcgcgtcgcgtcacacagcttggccacaatgtggtttttgtcaaacgaagattctatgacgtgtttaaagtttaggtcgagtaaagcgcaaatcttttttaaccctagaaagatagtctgcgtaaaattgacgcatgcattcttgaaatattgctctctctttctaaatagcgcgaatccgtcgctgtgcatttaggacatctcagtcgccgcttggagctcccgtgaggcgtgcttgtcaatgcggtaagtgtcactgattttgaactataacgaccgcgtgagtcaaaatgacgcatgattatcttttacgtgacttttaagatttaactcatacgataattatattgttatttcatgttctacttacgtgataacttattatatatatattttcttgttatagatatcatcaactttgtatagaaaagttgctcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattgcaagtttgtacaaaaaagcaggctgaatgccgccaccatggctaccgagtataaaccgacggtccgacttgcgactcgggatgacgttccacgcgccgtgaggactctggccgccgccttcgctgactacccggccacacggcacacagttgaccccgacagacacatagagcgggtcacagagttgcaagaacttttcctgacgcgggttggactcgatattggaaaagtttgggttgccgacgatggtgccgcagttgcggtgtggactactccagaaagtgtagaagctggagccgtattcgctgaaatagggccacggatggcagagctgtctggtagcaggctcgcagcgcagcagcagatggaaggccttctcgctccgcatcggcctaaggaaccggcgtggttcctggctacggtgggagtctcacctgaccatcaaggaaagggattgggaagtgctgtcgttcttccaggcgtggaagcggccgaacgagcaggcgtcccagccttcttggaaacgagcgcacccaggaatcttccattctacgagcgactgggtttcactgtgacagcagacgtagaagtccctgaagggcctcggacttggtgcatgaccagaaagcctggggcttgaaacgccatgatccagacatgataagatacattgatgagtttggacaaaccacaactagaatgcagtgaggcgcgcctggatacttctgcctgcaggggagtcgctcttttgcaatgctctttgggaaattatattagcctaattactaatttcctggccctcaaggtgacttgcttgaacttgccaccttcgccactagggggcagcattggtttacacagggtgaacaaacgttcaattctaaactaatattctttggtgggaaagtgtgttcattttggtttgttttgaagaagtcacttcatacctttgaactggggttaagctggttaactcccaaaattccaccagttccaatatcctatggaaaaaccccaaaaccactcaaaccagatatactcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagaaccaagcgaatgccgccaccatggctgtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaaacgccatgaattcactcctcaggtgcaggctgcctatcagaaggtggtggctggtgtggccaatgccctggctcacaaataccactgagatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgcccatatgctggctgccatgaacaaaggttggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatggagatccagtggcatcgctcttttgcaatgctctttgggaaattatattagcctaattactaatttcctggccctcaaggtgacttgcttgaacttgccaccttcgccactagggggcagcattggtttacacagggtgaacaaacgttcaattctaaactaatattctttggtgggaaagtgtgttcattttggtttgttttgaagaagtcacttcatacctttgaactggggttaagctggttaactcccaaaattccaccagttccaatatcctatggaaaaaccccaaaaccactcaaaccagatatactcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagaaccaagcgaatgccgccaccatggctgtcagtaaaggggaagagcttataaaggaaaatatgcacatgaagctctacatggagggcactgtagataaccaccatttcaaatgtacctctgaaggggagggcaagccatacgaaggtactcaaaccatgcgaataaaagtagttgaaggcgggcctcttccctttgcattcgacattctcgcaacctcttttctgtacggcagtaagactttcataaaccacactcaaggcattccagacttcttcaagcaatcattccccgaaggattcacctgggagcgagttactacttatgaggacggaggagtccttactgcaacccaagacacctcactgcaagatgggtgcctgatttacaatgtaaagatcagaggggtgaatttcacaagcaatgggccagttatgcaaaaaaagacccttggatgggaggccttcaccgagacactgtacccagccgatggtggactggagggcaggaatgacatggccctcaagctcgtcggaggcagtcacttgattgccaacgccaaaaccacttaccgctctaagaaacctgctaaaaacctgaagatgcccggcgtctattatgtggactatcgacttgagagaattaaggaggcaaacaacgagacttatgtcgaacagcatgaagttgccgtggctaggtactgtgatttgcccagcaaattgggtcataaacttaactgaaacgccatgaattcactcctcaggtgcaggctgcctatcagaaggtggtggctggtgtggccaatgccctggctcacaaataccactgagatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgcccatatgctggctgccatgaacaaaggttggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatggagatccagttcgcgcggccgcgcctgggcttacttaattaacgagagcataatattgatatgtgccaaagttgtttctgactgactaataagtataatttgtttctattatgtataggttaagctaattacttattttataatacaacatgactgtttttaaagtacaaaataagtttatttttgtaaaagagagaatgtttaaaagttttgttactttatagaagaaattttgagtttttgtttttttttaataaataaataaacataaataaattgtttgttgaatttattattagtatgtaagtgtaaatataataaaacttaatatctattcaaattaataaataaacctcgatatacagaccgataaaacacatgcgtcaattttacgcatgattatctttaacgtacgtcacaatatgattatctttctagggttaaataatagtttctaatttttttattattcagcctgctgtcgtgaataccgagctccaattcgccctatagtgagtcgtattacaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttg

SEQ ID NO: 24 - DNA, 10995 bp, synthetic construct
  source 1..10995: mol_type = other DNA
tgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctcgaaattaaccctcactaaagggaacaaaagctggtacctcgcgcgacttggtttgccattctttagcgcgcgtcgcgtcacacagcttggccacaatgtggtttttgtcaaacgaagattctatgacgtgtttaaagtttaggtcgagtaaagcgcaaatcttttttaaccctagaaagatagtctgcgtaaaattgacgcatgcattcttgaaatattgctctctctttctaaatagcgcgaatccgtcgctgtgcatttaggacatctcagtcgccgcttggagctcccgtgaggcgtgcttgtcaatgcggtaagtgtcactgattttgaactataacgaccgcgtgagtcaaaatgacgcatgattatcttttacgtgacttttaagatttaactcatacgataattatattgttatttcatgttctacttacgtgataacttattatatatatattttcttgttatagatatcatcaactttgtatagaaaagttgctcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattgcaagtttgtacaaaaaagcaggctgaatgccgccaccatggctaccgagtataaaccgacggtccgacttgcgactcgggatgacgttccacgcgccgtgaggactctggccgccgccttcgctgactacccggccacacggcacacagttgaccccgacagacacatagagcgggtcacagagttgcaagaacttttcctgacgcgggttggactcgatattggaaaagtttgggttgccgacgatggtgccgcagttgcggtgtggactactccagaaagtgtagaagctggagccgtattcgctgaaatagggccacggatggcagagctgtctggtagcaggctcgcagcgcagcagcagatggaaggccttctcgctccgcatcggcctaaggaaccggcgtggttcctggctacggtgggagtctcacctgaccatcaaggaaagggattgggaagtgctgtcgttcttccaggcgtggaagcggccgaacgagcaggcgtcccagccttcttggaaacgagcgcacccaggaatcttccattctacgagcgactgggtttcactgtgacagcagacgtagaagtccctgaagggcctcggacttggtgcatgaccagaaagcctggggcttgaaacgccatgatccagacatgataagatacattgatgagtttggacaaaccacaactagaatgcagtgaggcgcgcctggatacttctgcctgcaggggagtcgctcttttgcaatgctctttgggaaattatattagcctaattactaatttcctggccctcaaggtgacttgcttgaacttgccaccttcgccactagggggcagcattggtttacacagggtgaacaaacgttcaattctaaactaatattctttggtgggaaagtgtgttcattttggtttgttttgaagaagtcacttcatacctttgaactggggttaagctggttaactcccaaaattccaccagttccaatatcctatggaaaaaccccaaaaccactcaaaccagatatactcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagaaccaagcgaatgccgccaccatggctatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaaacgccatgaattcactcctcaggtgcaggctgcctatcagaaggtggtggctggtgtggccaatgccctggctcacaaataccactgagatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgcccatatgctggctgccatgaacaaaggttggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatggagatcagtggcatcgctcttttgcaatgctctttgggaaattatattagcctaattactaatttcctggccctcaaggtgacttgcttgaacttgccaccttcgccactagggggcagcattggtttacacagggtgaacaaacgttcaattctaaactaatattctttggtgggaaagtgtgttcattttggtttgttttgaagaagtcacttcatacctttgaactggggttaagctggttaactcccaaaattccaccagttccaatatcctatggaaaaaccccaaaaccactcaaaccagatatactcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagaaccaagcgaatgccgccaccatggctgctgaaggttccgtcgccaggcagcctgacctcttgacctgcgacgatgagccgatccatatccccggtgccatccaaccgcatggactgctgctcgccctcgccgccgacatgacgatcgttgccggcagcgacaaccttcccgaactcaccggactggcgatcggcgccctgatcggccgctctgcggccgatgtgttcgactcggaaacgcacaaccgtctgacgatcgccttggccgagcccggggcggccgtcggagcaccgatcactgtcggcttcacgatgcgaaaggacgcaggcttcatcggctcctggcatcgccatgatcagctcatcttcctggagctggagcctccccagcgggacgtcgccgagccgcaggcgttcttccgccgcaccaacagcgccatccgccgcctgcaggccgccgaaaccttggaaagcgcctgcgccgccgcggcgcaagaggtgcggaagattaccggcttcgatcgggtgatgatctatcgcttcgcctccgacttcagcggcgaagtgatcgcagaggatcggtgcgccgaggtcgagtcaaaactaggcctgcactatcctgcctcaaccgtgccggcgcaggcccgtcggctctataccatcaacccggtacggatcattcccgatatcaattatcggccggtgccggtcaccccagacctcaatccggtcaccgggcggccgattgatcttagcttcgccatcctgcgcagcgtgtcgcccgtccatctggagttcatgcgcaacataggcatgcacggcacgatgtcgatctcgattttgcgcggcgagcgactgtggggattgatcgtttgccatcaccgaacgccgtactacgtcgatctcgatggccgccaagcctgcgagctagtcgcccaggttctggcctggcagatcggcgtgatggaagagtaaaacgccatgaattcactcctcaggtgcaggctgcctatcagaaggtggtggctggtgtggccaatgccctggctcacaaataccactgagatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgcccatatgctggctgccatgaacaaaggttggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatggagatcagttcgcgcggccgcgcctgggcttacttaattaacgagagcataatattgatatgtgccaaagttgtttctgactgactaataagtataatttgtttctattatgtataggttaagctaattacttattttataatacaacatgactgtttttaaagtacaaaataagtttatttttgtaaaagagagaatgtttaaaagttttgttactttatagaagaaattttgagtttttgtttttttttaataaataaataaacataaataaattgtttgttgaatttattattagtatgtaagtgtaaatataataaaacttaatatctattcaaattaataaataaacctcgatatacagaccgataaaacacatgcgtcaattttacgcatgattatctttaacgtacgtcacaatatgattatctttctagggttaaataatagtttctaatttttttattattcagcctgctgtcgtgaataccgagctccaattcgccctatagtgagtcgtattacaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtat

SEQ ID NO: 25 - DNA, 10748 bp, synthetic construct
  source 1..10748: mol_type = other DNA
caggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctcgaaattaaccctcactaaagggaacaaaagctggtacctcgcgcgacttggtttgccattctttagcgcgcgtcgcgtcacacagcttggccacaatgtggtttttgtcaaacgaagattctatgacgtgtttaaagtttaggtcgagtaaagcgcaaatcttttttaaccctagaaagatagtctgcgtaaaattgacgcatgcattcttgaaatattgctctctctttctaaatagcgcgaatccgtcgctgtgcatttaggacatctcagtcgccgcttggagctcccgtgaggcgtgcttgtcaatgcggtaagtgtcactgattttgaactataacgaccgcgtgagtcaaaatgacgcatgattatcttttacgtgacttttaagatttaactcatacgataattatattgttatttcatgttctacttacgtgataacttattatatatatattttcttgttatagatatcatcaactttgtatagaaaagttgctcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattgcaagtttgtacaaaaaagcaggctgaatgccgccaccatggctaccgagtataaaccgacggtccgacttgcgactcgggatgacgttccacgcgccgtgaggactctggccgccgccttcgctgactacccggccacacggcacacagttgaccccgacagacacatagagcgggtcacagagttgcaagaacttttcctgacgcgggttggactcgatattggaaaagtttgggttgccgacgatggtgccgcagttgcggtgtggactactccagaaagtgtagaagctggagccgtattcgctgaaatagggccacggatggcagagctgtctggtagcaggctcgcagcgcagcagcagatggaaggccttctcgctccgcatcggcctaaggaaccggcgtggttcctggctacggtgggagtctcacctgaccatcaaggaaagggattgggaagtgctgtcgttcttccaggcgtggaagcggccgaacgagcaggcgtcccagccttcttggaaacgagcgcacccaggaatcttccattctacgagcgactgggtttcactgtgacagcagacgtagaagtccctgaagggcctcggacttggtgcatgaccagaaagcctggggcttgaaacgccatgatccagacatgataagatacattgatgagtttggacaaaccacaactagaatgcagtgaggcgcgcctggatacttctgcctgcaggggagtcgctcttttgcaatgctctttgggaaattatattagcctaattactaatttcctggccctcaaggtgacttgcttgaacttgccaccttcgccactagggggcagcattggtttacacagggtgaacaaacgttcaattctaaactaatattctttggtgggaaagtgtgttcattttggtttgttttgaagaagtcacttcatacctttgaactggggttaagctggttaactcccaaaattccaccagttccaatatcctatggaaaaaccccaaaaccactcaaaccagatatactcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagaaccaagcgaatgccgccaccatggctgtcagtaaaggggaagagcttataaaggaaaatatgcacatgaagctctacatggagggcactgtagataaccaccatttcaaatgtacctctgaaggggagggcaagccatacgaaggtactcaaaccatgcgaataaaagtagttgaaggcgggcctcttccctttgcattcgacattctcgcaacctcttttctgtacggcagtaagactttcataaaccacactcaaggcattccagacttcttcaagcaatcattccccgaaggattcacctgggagcgagttactacttatgaggacggaggagtccttactgcaacccaagacacctcactgcaagatgggtgcctgatttacaatgtaaagatcagaggggtgaatttcacaagcaatgggccagttatgcaaaaaaagacccttggatgggaggccttcaccgagacactgtacccagccgatggtggactggagggcaggaatgacatggccctcaagctcgtcggaggcagtcacttgattgccaacgccaaaaccacttaccgctctaagaaacctgctaaaaacctgaagatgcccggcgtctattatgtggactatcgacttgagagaattaaggaggcaaacaacgagacttatgtcgaacagcatgaagttgccgtggctaggtactgtgatttgcccagcaaattgggtcataaacttaacaacgccatgaattcactcctcaggtgcaggctgcctatcagaaggtggtggctggtgtggccaatgccctggctcacaaataccactgagatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgcccatatgctggctgccatgaacaaaggttggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatggagatccagtggcatcgctcttttgcaatgctctttgggaaattatattagcctaattactaatttcctggccctcaaggtgacttgcttgaacttgccaccttcgccactagggggcagcattggtttacacagggtgaacaaacgttcaattctaaactaatattctttggtgggaaagtgtgttcattttggtttgttttgaagaagtcacttcatacctttgaactggggttaagctggttaactcccaaaattccaccagttccaatatcctatggaaaaaccccaaaaccactcaaaccagatatactcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagaaccaagcgaatgccgccaccatggctgtcagtaaaggggaagagcttataaaggaaaatatgcacatgaagctctacatggagggcactgtagataaccaccatttcaaatgtacctctgaaggggagggcaagccatacgaaggtactcaaaccatgcgaataaaagtagttgaaggcgggcctcttccctttgcattcgacattctcgcaacctcttttctgtacggcagtaagactttcataaaccacactcaaggcattccagacttcttcaagcaatcattccccgaaggattcacctgggagcgagttactacttatgaggacggaggagtccttactgcaacccaagacacctcactgcaagatgggtgcctgatttacaatgtaaagatcagaggggtgaatttcacaagcaatgggccagttatgcaaaaaaagacccttggatgggaggccttcaccgagacactgtacccagccgatggtggactggagggcaggaatgacatggccctcaagctcgtcggaggcagtcacttgattgccaacgccaaaaccacttaccgctctaagaaacctgctaaaaacctgaagatgcccggcgtctattatgtggactatcgacttgagagaattaaggaggcaaacaacgagacttatgtcgaacagcatgaagttgccgtggctaggtactgtgatttgcccagcaaattgggtcataaacttaactgaaacgccatgaattcactcctcaggtgcaggctgcctatcagaaggtggtggctggtgtggccaatgccctggctcacaaataccactgagatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgcccatatgctggctgccatgaacaaaggttggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatggagatccagttcgcgcggccgcgcctgggcttacttaattaacgagagcataatattgatatgtgccaaagttgtttctgactgactaataagtataatttgtttctattatgtataggttaagctaattacttattttataatacaacatgactgtttttaaagtacaaaataagtttatttttgtaaaagagagaatgtttaaaagttttgttactttatagaagaaattttgagtttttgtttttttttaataaataaataaacataaataaattgtttgttgaatttattattagtatgtaagtgtaaatataataaaacttaatatctattcaaattaataaataaacctcgatatacagaccgataaaacacatgcgtcaattttacgcatgattatctttaacgtacgtcacaatatgattatctttctagggttaaataatagtttctaatttttttattattcagcctgctgtcgtgaataccgagctccaattcgccctatagtgagtcgtattacaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttg

SEQ ID NO: 26 - DNA, 13232 bp, synthetic construct
  source 1..13232: mol_type = other DNA
caggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctcgaaattaaccctcactaaagggaacaaaagctggtacctcgcgcgacttggtttgccattctttagcgcgcgtcgcgtcacacagcttggccacaatgtggtttttgtcaaacgaagattctatgacgtgtttaaagtttaggtcgagtaaagcgcaaatcttttttaaccctagaaagatagtctgcgtaaaattgacgcatgcattcttgaaatattgctctctctttctaaatagcgcgaatccgtcgctgtgcatttaggacatctcagtcgccgcttggagctcccgtgaggcgtgcttgtcaatgcggtaagtgtcactgattttgaactataacgaccgcgtgagtcaaaatgacgcatgattatcttttacgtgacttttaagatttaactcatacgataattatattgttatttcatgttctacttacgtgataacttattatatatatattttcttgttatagatatcatcaactttgtatagaaaagttgctcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattgcaagtttgtacaaaaaagcaggctgaatgccgccaccatggctaccgagtataaaccgacggtccgacttgcgactcgggatgacgttccacgcgccgtgaggactctggccgccgccttcgctgactacccggccacacggcacacagttgaccccgacagacacatagagcgggtcacagagttgcaagaacttttcctgacgcgggttggactcgatattggaaaagtttgggttgccgacgatggtgccgcagttgcggtgtggactactccagaaagtgtagaagctggagccgtattcgctgaaatagggccacggatggcagagctgtctggtagcaggctcgcagcgcagcagcagatggaaggccttctcgctccgcatcggcctaaggaaccggcgtggttcctggctacggtgggagtctcacctgaccatcaaggaaagggattgggaagtgctgtcgttcttccaggcgtggaagcggccgaacgagcaggcgtcccagccttcttggaaacgagcgcacccaggaatcttccattctacgagcgactgggtttcactgtgacagcagacgtagaagtccctgaagggcctcggacttggtgcatgaccagaaagcctggggcttgaaacgccatgatccagacatgataagatacattgatgagtttggacaaaccacaactagaatgcagtgaggcgcgcctggatacttctgcctgcaggggagtcgctcttttgcaatgctctttgggaaattatattagcctaattactaatttcctggccctcaaggtgacttgcttgaacttgccaccttcgccactagggggcagcattggtttacacagggtgaacaaacgttcaattctaaactaatattctttggtgggaaagtgtgttcattttggtttgttttgaagaagtcacttcatacctttgaactggggttaagctggttaactcccaaaattccaccagttccaatatcctatggaaaaaccccaaaaccactcaaaccagatatactcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagaaccaagcgaatgccgccaccatggctgtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaaacgccatgaattcactcctcaggtgcaggctgcctatcagaaggtggtggctggtgtggccaatgccctggctcacaaataccactgagatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgcccatatgctggctgccatgaacaaaggttggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatggagatccagtggcatcgctcttttgcaatgctctttgggaaattatattagcctaattactaatttcctggccctcaaggtgacttgcttgaacttgccaccttcgccactagggggcagcattggtttacacagggtgaacaaacgttcaattctaaactaatattctttggtgggaaagtgtgttcattttggtttgttttgaagaagtcacttcatacctttgaactggggttaagctggttaactcccaaaattccaccagttccaatatcctatggaaaaaccccaaaaccactcaaaccagatatactcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagaaccaagcgaatgccgccaccatggctgtcagtaaaggggaagagcttataaaggaaaatatgcacatgaagctctacatggagggcactgtagataaccaccatttcaaatgtacctctgaaggggagggcaagccatacgaaggtactcaaaccatgcgaataaaagtagttgaaggcgggcctcttccctttgcattcgacattctcgcaacctcttttctgtacggcagtaagactttcataaaccacactcaaggcattccagacttcttcaagcaatcattccccgaaggattcacctgggagcgagttactacttatgaggacggaggagtccttactgcaacccaagacacctcactgcaagatgggtgcctgatttacaatgtaaagatcagaggggtgaatttcacaagcaatgggccagttatgcaaaaaaagacccttggatgggaggccttcaccgagacactgtacccagccgatggtggactggagggcaggaatgacatggccctcaagctcgtcggaggcagtcacttgattgccaacgccaaaaccacttaccgctctaagaaacctgctaaaaacctgaagatgcccggcgtctattatgtggactatcgacttgagagaattaaggaggcaaacaacgagacttatgtcgaacagcatgaagttgccgtggctaggtactgtgatttgcccagcaaattgggtcataaacttaactgaaacgccatgaattcactcctcaggtgcaggctgcctatcagaaggtggtggctggtgtggccaatgccctggctcacaaataccactgagatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgcccatatgctggctgccatgaacaaaggttggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatggagatccagtnnnntcgctcttttgcaatgctctttgggaaattatattagcctaattactaatttcctggccctcaaggtgacttgcttgaacttgccaccttcgccactagggggcagcattggtttacacagggtgaacaaacgttcaattctaaactaatattctttggtgggaaagtgtgttcattttggtttgttttgaagaagtcacttcatacctttgaactggggttaagctggttaactcccaaaattccaccagttccaatatcctatggaaaaaccccaaaaccactcaaaccagatatactcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagaaccaagcgaatgccgccaccatggctgctgaaggttccgtcgccaggcagcctgacctcttgacctgcgacgatgagccgatccatatccccggtgccatccaaccgcatggactgctgctcgccctcgccgccgacatgacgatcgttgccggcagcgacaaccttcccgaactcaccggactggcgatcggcgccctgatcggccgctctgcggccgatgtgttcgactcggaaacgcacaaccgtctgacgatcgccttggccgagcccggggcggccgtcggagcaccgatcactgtcggcttcacgatgcgaaaggacgcaggcttcatcggctcctggcatcgccatgatcagctcatcttcctggagctggagcctccccagcgggacgtcgccgagccgcaggcgttcttccgccgcaccaacagcgccatccgccgcctgcaggccgccgaaaccttggaaagcgcctgcgccgccgcggcgcaagaggtgcggaagattaccggcttcgatcgggtgatgatctatcgcttcgcctccgacttcagcggcgaagtgatcgcagaggatcggtgcgccgaggtcgagtcaaaactaggcctgcactatcctgcctcaaccgtgccggcgcaggcccgtcggctctataccatcaacccggtacggatcattcccgatatcaattatcggccggtgccggtcaccccagacctcaatccggtcaccgggcggccgattgatcttagcttcgccatcctgcgcagcgtgtcgcccgtccatctggagttcatgcgcaacataggcatgcacggcacgatgtcgatctcgattttgcgcggcgagcgactgtggggattgatcgtttgccatcaccgaacgccgtactacgtcgatctcgatggccgccaagcctgcgagctagtcgcccaggttctggcctggcagatcggcgtgatggaagagtaaaacgccatgaattcactcctcaggtgcaggctgcctatcagaaggtggtggctggtgtggccaatgccctggctcacaaataccactgagatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgcccatatgctggctgccatgaacaaaggttggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatggagatccagtnnnngcggccgcgcctgggcttacttaattaacgagagcataatattgatatgtgccaaagttgtttctgactgactaataagtataatttgtttctattatgtataggttaagctaattacttattttataatacaacatgactgtttttaaagtacaaaataagtttatttttgtaaaagagagaatgtttaaaagttttgttactttatagaagaaattttgagtttttgtttttttttaataaataaataaacataaataaattgtttgttgaatttattattagtatgtaagtgtaaatataataaaacttaatatctattcaaattaataaataaacctcgatatacagaccgataaaacacatgcgtcaattttacgcatgattatctttaacgtacgtcacaatatgattatctttctagggttaaataatagtttctaatttttttattattcagcctgctgtcgtgaataccgagctccaattcgccctatagtgagtcgtattacaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttg

SEQ ID NO: 27 - DNA, 15476 bp, synthetic construct
  source 1..15476: mol_type = other DNA
caggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctcgaaattaaccctcactaaagggaacaaaagctggtacctcgcgcgacttggtttgccattctttagcgcgcgtcgcgtcacacagcttggccacaatgtggtttttgtcaaacgaagattctatgacgtgtttaaagtttaggtcgagtaaagcgcaaatcttttttaaccctagaaagatagtctgcgtaaaattgacgcatgcattcttgaaatattgctctctctttctaaatagcgcgaatccgtcgctgtgcatttaggacatctcagtcgccgcttggagctcccgtgaggcgtgcttgtcaatgcggtaagtgtcactgattttgaactataacgaccgcgtgagtcaaaatgacgcatgattatcttttacgtgacttttaagatttaactcatacgataattatattgttatttcatgttctacttacgtgataacttattatatatatattttcttgttatagatatcatcaactttgtatagaaaagttgctcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattgcaagtttgtacaaaaaagcaggctgaatgccgccaccatggctaccgagtataaaccgacggtccgacttgcgactcgggatgacgttccacgcgccgtgaggactctggccgccgccttcgctgactacccggccacacggcacacagttgaccccgacagacacatagagcgggtcacagagttgcaagaacttttcctgacgcgggttggactcgatattggaaaagtttgggttgccgacgatggtgccgcagttgcggtgtggactactccagaaagtgtagaagctggagccgtattcgctgaaatagggccacggatggcagagctgtctggtagcaggctcgcagcgcagcagcagatggaaggccttctcgctccgcatcggcctaaggaaccggcgtggttcctggctacggtgggagtctcacctgaccatcaaggaaagggattgggaagtgctgtcgttcttccaggcgtggaagcggccgaacgagcaggcgtcccagccttcttggaaacgagcgcacccaggaatcttccattctacgagcgactgggtttcactgtgacagcagacgtagaagtccctgaagggcctcggacttggtgcatgaccagaaagcctggggcttgaaacgccatgatccagacatgataagatacattgatgagtttggacaaaccacaactagaatgcagtgaggcgcgcctggatacttctgcctgcaggggagtcgctcttttgcaatgctctttgggaaattatattagcctaattactaatttcctggccctcaaggtgacttgcttgaacttgccaccttcgccactagggggcagcattggtttacacagggtgaacaaacgttcaattctaaactaatattctttggtgggaaagtgtgttcattttggtttgttttgaagaagtcacttcatacctttgaactggggttaagctggttaactcccaaaattccaccagttccaatatcctatggaaaaaccccaaaaccactcaaaccagatatactcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagaaccaagcgaatgccgccaccatggctgtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaaacgccatgaattcactcctcaggtgcaggctgcctatcagaaggtggtggctggtgtggccaatgccctggctcacaaataccactgagatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgcccatatgctggctgccatgaacaaaggttggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatggagatccagtggcatcgctcttttgcaatgctctttgggaaattatattagcctaattactaatttcctggccctcaaggtgacttgcttgaacttgccaccttcgccactagggggcagcattggtttacacagggtgaacaaacgttcaattctaaactaatattctttggtgggaaagtgtgttcattttggtttgttttgaagaagtcacttcatacctttgaactggggttaagctggttaactcccaaaattccaccagttccaatatcctatggaaaaaccccaaaaccactcaaaccagatatactcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagaaccaagcgaatgccgccaccatggctgtcagtaaaggggaagagcttataaaggaaaatatgcacatgaagctctacatggagggcactgtagataaccaccatttcaaatgtacctctgaaggggagggcaagccatacgaaggtactcaaaccatgcgaataaaagtagttgaaggcgggcctcttccctttgcattcgacattctcgcaacctcttttctgtacggcagtaagactttcataaaccacactcaaggcattccagacttcttcaagcaatcattccccgaaggattcacctgggagcgagttactacttatgaggacggaggagtccttactgcaacccaagacacctcactgcaagatgggtgcctgatttacaatgtaaagatcagaggggtgaatttcacaagcaatgggccagttatgcaaaaaaagacccttggatgggaggccttcaccgagacactgtacccagccgatggtggactggagggcaggaatgacatggccctcaagctcgtcggaggcagtcacttgattgccaacgccaaaaccacttaccgctctaagaaacctgctaaaaacctgaagatgcccggcgtctattatgtggactatcgacttgagagaattaaggaggcaaacaacgagacttatgtcgaacagcatgaagttgccgtggctaggtactgtgatttgcccagcaaattgggtcataaacttaactgaaacgccatgaattcactcctcaggtgcaggctgcctatcagaaggtggtggctggtgtggccaatgccctggctcacaaataccactgagatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgcccatatgctggctgccatgaacaaaggttggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatggagatccagtnnnntcgctcttttgcaatgctctttgggaaattatattagcctaattactaatttcctggccctcaaggtgacttgcttgaacttgccaccttcgccactagggggcagcattggtttacacagggtgaacaaacgttcaattctaaactaatattctttggtgggaaagtgtgttcattttggtttgttttgaagaagtcacttcatacctttgaactggggttaagctggttaactcccaaaattccaccagttccaatatcctatggaaaaaccccaaaaccactcaaaccagatatactcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagaaccaagcgaatgccgccaccatggctgctgaaggttccgtcgccaggcagcctgacctcttgacctgcgacgatgagccgatccatatccccggtgccatccaaccgcatggactgctgctcgccctcgccgccgacatgacgatcgttgccggcagcgacaaccttcccgaactcaccggactggcgatcggcgccctgatcggccgctctgcggccgatgtgttcgactcggaaacgcacaaccgtctgacgatcgccttggccgagcccggggcggccgtcggagcaccgatcactgtcggcttcacgatgcgaaaggacgcaggcttcatcggctcctggcatcgccatgatcagctcatcttcctggagctggagcctccccagcgggacgtcgccgagccgcaggcgttcttccgccgcaccaacagcgccatccgccgcctgcaggccgccgaaaccttggaaagcgcctgcgccgccgcggcgcaagaggtgcggaagattaccggcttcgatcgggtgatgatctatcgcttcgcctccgacttcagcggcgaagtgatcgcagaggatcggtgcgccgaggtcgagtcaaaactaggcctgcactatcctgcctcaaccgtgccggcgcaggcccgtcggctctataccatcaacccggtacggatcattcccgatatcaattatcggccggtgccggtcaccccagacctcaatccggtcaccgggcggccgattgatcttagcttcgccatcctgcgcagcgtgtcgcccgtccatctggagttcatgcgcaacataggcatgcacggcacgatgtcgatctcgattttgcgcggcgagcgactgtggggattgatcgtttgccatcaccgaacgccgtactacgtcgatctcgatggccgccaagcctgcgagctagtcgcccaggttctggcctggcagatcggcgtgatggaagagtaaaacgccatgaattcactcctcaggtgcaggctgcctatcagaaggtggtggctggtgtggccaatgccctggctcacaaataccactgagatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgcccatatgctggctgccatgaacaaaggttggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatggagatccagtnnnntcgctcttttgcaatgctctttgggaaattatattagcctaattactaatttcctggccctcaaggtgacttgcttgaacttgccaccttcgccactagggggcagcattggtttacacagggtgaacaaacgttcaattctaaactaatattctttggtgggaaagtgtgttcattttggtttgttttgaagaagtcacttcatacctttgaactggggttaagctggttaactcccaaaattccaccagttccaatatcctatggaaaaaccccaaaaccactcaaaccagatatactcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagaaccaagcgaatgccgccaccatggctggcaatagcgttagcaaaggtgaggaagataacatggcgattataaaggaatttatgcggttcaaggtgcacatggagggttcagttaatggacacgagttcgaaatcgaaggtgagggggaaggtagaccttacgaaggaactcagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccttgcaggacggcgagttcatctacaaggtgaagctgcgcggcaccaacttcccctccgacggccccgtaatgcagaagaaaaccatgggctggcaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtataagtaaaacgccatgaattcactcctcaggtgcaggctgcctatcagaaggtggtggctggtgtggccaatgccctggctcacaaataccactgagatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgcccatatgctggctgccatgaacaaaggttggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatggagatccagtnnnngcggccgcgcctgggcttacttaattaacgagagcataatattgatatgtgccaaagttgtttctgactgactaataagtataatttgtttctattatgtataggttaagctaattacttattttataatacaacatgactgtttttaaagtacaaaataagtttatttttgtaaaagagagaatgtttaaaagttttgttactttatagaagaaattttgagtttttgtttttttttaataaataaataaacataaataaattgtttgttgaatttattattagtatgtaagtgtaaatataataaaacttaatatctattcaaattaataaataaacctcgatatacagaccgataaaacacatgcgtcaattttacgcatgattatctttaacgtacgtcacaatatgattatctttctagggttaaataatagtttctaatttttttattattcagcctgctgtcgtgaataccgagctccaattcgccctatagtgagtcgtattacaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttg

SEQ ID NO: 28 - DNA, 6283 bp, synthetic construct
  source 1..6283: mol_type = other DNA
caggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctcgaaattaaccctcactaaagggaacaaaagctggtacctcgcgcgacttggtttgccattctttagcgcgcgtcgcgtcacacagcttggccacaatgtggtttttgtcaaacgaagattctatgacgtgtttaaagtttaggtcgagtaaagcgcaaatcttttttaaccctagaaagatagtctgcgtaaaattgacgcatgcattcttgaaatattgctctctctttctaaatagcgcgaatccgtcgctgtgcatttaggacatctcagtcgccgcttggagctcccgtgaggcgtgcttgtcaatgcggtaagtgtcactgattttgaactataacgaccgcgtgagtcaaaatgacgcatgattatcttttacgtgacttttaagatttaactcatacgataattatattgttatttcatgttctacttacgtgataacttattatatatatattttcttgttatagatatcatcaactttgtatagaaaagttgctcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattgcaagtttgtacaaaaaagcaggctgaatgccgccaccatggctaccgagtataaaccgacggtccgacttgcgactcgggatgacgttccacgcgccgtgaggactctggccgccgccttcgctgactacccggccacacggcacacagttgaccccgacagacacatagagcgggtcacagagttgcaagaacttttcctgacgcgggttggactcgatattggaaaagtttgggttgccgacgatggtgccgcagttgcggtgtggactactccagaaagtgtagaagctggagccgtattcgctgaaatagggccacggatggcagagctgtctggtagcaggctcgcagcgcagcagcagatggaaggccttctcgctccgcatcggcctaaggaaccggcgtggttcctggctacggtgggagtctcacctgaccatcaaggaaagggattgggaagtgctgtcgttcttccaggcgtggaagcggccgaacgagcaggcgtcccagccttcttggaaacgagcgcacccaggaatcttccattctacgagcgactgggtttcactgtgacagcagacgtagaagtccctgaagggcctcggacttggtgcatgaccagaaagcctggggcttgaaacgccatgatccagacatgataagatacattgatgagtttggacaaaccacaactagaatgcagtgaggcgcgcctggatacttctgcctgcaggttcattaacgacgcggccgcgcctgggcttacttaattaacgagagcataatattgatatgtgccaaagttgtttctgactgactaataagtataatttgtttctattatgtataggttaagctaattacttattttataatacaacatgactgtttttaaagtacaaaataagtttatttttgtaaaagagagaatgtttaaaagttttgttactttatagaagaaattttgagtttttgtttttttttaataaataaataaacataaataaattgtttgttgaatttattattagtatgtaagtgtaaatataataaaacttaatatctattcaaattaataaataaacctcgatatacagaccgataaaacacatgcgtcaattttacgcatgattatctttaacgtacgtcacaatatgattatctttctagggttaaataatagtttctaatttttttattattcagcctgctgtcgtgaataccgagctccaattcgccctatagtgagtcgtattacaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttg

SEQ ID NO: 29 - DNA, 10754 bp, synthetic construct
  source 1..10754: mol_type = other DNA
gcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctcgaaattaaccctcactaaagggaacaaaagctggtacctcgcgcgacttggtttgccattctttagcgcgcgtcgcgtcacacagcttggccacaatgtggtttttgtcaaacgaagattctatgacgtgtttaaagtttaggtcgagtaaagcgcaaatcttttttaaccctagaaagatagtctgcgtaaaattgacgcatgcattcttgaaatattgctctctctttctaaatagcgcgaatccgtcgctgtgcatttaggacatctcagtcgccgcttggagctcccgtgaggcgtgcttgtcaatgcggtaagtgtcactgattttgaactataacgaccgcgtgagtcaaaatgacgcatgattatcttttacgtgacttttaagatttaactcatacgataattatattgttatttcatgttctacttacgtgataacttattatatatatattttcttgttatagatatcatcaactttgtatagaaaagttgctcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattgcaagtttgtacaaaaaagcaggctgaatgccgccaccatggctaccgagtataaaccgacggtccgacttgcgactcgggatgacgttccacgcgccgtgaggactctggccgccgccttcgctgactacccggccacacggcacacagttgaccccgacagacacatagagcgggtcacagagttgcaagaacttttcctgacgcgggttggactcgatattggaaaagtttgggttgccgacgatggtgccgcagttgcggtgtggactactccagaaagtgtagaagctggagccgtattcgctgaaatagggccacggatggcagagctgtctggtagcaggctcgcagcgcagcagcagatggaaggccttctcgctccgcatcggcctaaggaaccggcgtggttcctggctacggtgggagtctcacctgaccatcaaggaaagggattgggaagtgctgtcgttcttccaggcgtggaagcggccgaacgagcaggcgtcccagccttcttggaaacgagcgcacccaggaatcttccattctacgagcgactgggtttcactgtgacagcagacgtagaagtccctgaagggcctcggacttggtgcatgaccagaaagcctggggcttgaaacgccatgatccagacatgataagatacattgatgagtttggacaaaccacaactagaatgcagtgaggcgcgcctggatacttctgcctgcaggggagtcgctcttttgcaatgctctttgggaaattatattagcctaattactaatttcctggccctcaaggtgacttgcttgaacttgccaccttcgccactagggggcagcattggtttacacagggtgaacaaacgttcaattctaaactaatattctttggtgggaaagtgtgttcattttggtttgttttgaagaagtcacttcatacctttgaactggggttaagctggttaactcccaaaattccaccagttccaatatcctatggaaaaaccccaaaaccactcaaaccagatatactcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagaaccaagcgaatgccgccaccatgcacagttcagctctgctgtgctgcctcgtacttctcaccggcgtcagagcaagcccaggccagggcacccaatctgagaattcctgtacacatttccctgggaatcttcccaatatgctgcgcgacctgcgggatgccttctctcgcgttaagacttttttccagatgaaagatcagctggacaacctcttactgaaggaatcattgctcgaggattttaagggatacttaggttgccaggcgttgtccgagatgatccagttttacctggaagaggtgatgcctcaagcagaaaaccaagaccccgacatcaaagcccacgtgaatagcctaggagaaaacttgaaaacgctccgtctgcgactgcggagatgtcataggtttctaccatgtgagaacaaaagcaaggccgtcgagcaggtgaagaacgccttcaacaaacttcaggagaaagggatttataaggctatgagtgaattcgatatatttatcaattatattgaggcttacatgacaatgaagattaggaattaaaacgccatgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagagaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagtcggggtttcaccatattggccaggctggtgtccaactcctaatctcaggtgatctacccaccttggcctcccaaattgctgggattacaggcgtgaaccactgctcccttccctgtccttcagtggcatcgctcttttgcaatgctctttgggaaattatattagcctaattactaatttcctggccctcaaggtgacttgcttgaacttgccaccttcgccactagggggcagcattggtttacacagggtgaacaaacgttcaattctaaactaatattctttggtgggaaagtgtgttcattttggtttgttttgaagaagtcacttcatacctttgaactggggttaagctggttaactcccaaaattccaccagttccaatatcctatggaaaaaccccaaaaccactcaaaccagatatactcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagaaccaagcgaatgccgccaccatggctgctgaaggttccgtcgccaggcagcctgacctcttgacctgcgacgatgagccgatccatatccccggtgccatccaaccgcatggactgctgctcgccctcgccgccgacatgacgatcgttgccggcagcgacaaccttcccgaactcaccggactggcgatcggcgccctgatcggccgctctgcggccgatgtgttcgactcggaaacgcacaaccgtctgacgatcgccttggccgagcccggggcggccgtcggagcaccgatcactgtcggcttcacgatgcgaaaggacgcaggcttcatcggctcctggcatcgccatgatcagctcatcttcctggagctggagcctccccagcgggacgtcgccgagccgcaggcgttcttccgccgcaccaacagcgccatccgccgcctgcaggccgccgaaaccttggaaagcgcctgcgccgccgcggcgcaagaggtgcggaagattaccggcttcgatcgggtgatgatctatcgcttcgcctccgacttcagcggcgaagtgatcgcagaggatcggtgcgccgaggtcgagtcaaaactaggcctgcactatcctgcctcaaccgtgccggcgcaggcccgtcggctctataccatcaacccggtacggatcattcccgatatcaattatcggccggtgccggtcaccccagacctcaatccggtcaccgggcggccgattgatcttagcttcgccatcctgcgcagcgtgtcgcccgtccatctggagttcatgcgcaacataggcatgcacggcacgatgtcgatctcgattttgcgcggcgagcgactgtggggattgatcgtttgccatcaccgaacgccgtactacgtcgatctcgatggccgccaagcctgcgagctagtcgcccaggttctggcctggcagatcggcgtgatggaagagtaaaacgccatgaattcactcctcaggtgcaggctgcctatcagaaggtggtggctggtgtggccaatgccctggctcacaaataccactgagatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgcccatatgctggctgccatgaacaaaggttggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatggagatcagttcgcgcggccgcgcctgggcttacttaattaacgagagcataatattgatatgtgccaaagttgtttctgactgactaataagtataatttgtttctattatgtataggttaagctaattacttattttataatacaacatgactgtttttaaagtacaaaataagtttatttttgtaaaagagagaatgtttaaaagttttgttactttatagaagaaattttgagtttttgtttttttttaataaataaataaacataaataaattgtttgttgaatttattattagtatgtaagtgtaaatataataaaacttaatatctattcaaattaataaataaacctcgatatacagaccgataaaacacatgcgtcaattttacgcatgattatctttaacgtacgtcacaatatgattatctttctagggttaaataatagtttctaatttttttattattcagcctgctgtcgtgaataccgagctccaattcgccctatagtgagtcgtattacaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggc

SEQ ID NO: 30 - DNA, 10626 bp, synthetic construct
  source 1..10626: mol_type = other DNA
gcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctcgaaattaaccctcactaaagggaacaaaagctggtacctcgcgcgacttggtttgccattctttagcgcgcgtcgcgtcacacagcttggccacaatgtggtttttgtcaaacgaagattctatgacgtgtttaaagtttaggtcgagtaaagcgcaaatcttttttaaccctagaaagatagtctgcgtaaaattgacgcatgcattcttgaaatattgctctctctttctaaatagcgcgaatccgtcgctgtgcatttaggacatctcagtcgccgcttggagctcccgtgaggcgtgcttgtcaatgcggtaagtgtcactgattttgaactataacgaccgcgtgagtcaaaatgacgcatgattatcttttacgtgacttttaagatttaactcatacgataattatattgttatttcatgttctacttacgtgataacttattatatatatattttcttgttatagatatcatcaactttgtatagaaaagttgctcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattgcaagtttgtacaaaaaagcaggctgaatgccgccaccatggctaccgagtataaaccgacggtccgacttgcgactcgggatgacgttccacgcgccgtgaggactctggccgccgccttcgctgactacccggccacacggcacacagttgaccccgacagacacatagagcgggtcacagagttgcaagaacttttcctgacgcgggttggactcgatattggaaaagtttgggttgccgacgatggtgccgcagttgcggtgtggactactccagaaagtgtagaagctggagccgtattcgctgaaatagggccacggatggcagagctgtctggtagcaggctcgcagcgcagcagcagatggaaggccttctcgctccgcatcggcctaaggaaccggcgtggttcctggctacggtgggagtctcacctgaccatcaaggaaagggattgggaagtgctgtcgttcttccaggcgtggaagcggccgaacgagcaggcgtcccagccttcttggaaacgagcgcacccaggaatcttccattctacgagcgactgggtttcactgtgacagcagacgtagaagtccctgaagggcctcggacttggtgcatgaccagaaagcctggggcttgaaacgccatgatccagacatgataagatacattgatgagtttggacaaaccacaactagaatgcagtgaggcgcgcctggatacttctgcctgcaggggagtcgctcttttgcaatgctctttgggaaattatattagcctaattactaatttcctggccctcaaggtgacttgcttgaacttgccaccttcgccactagggggcagcattggtttacacagggtgaacaaacgttcaattctaaactaatattctttggtgggaaagtgtgttcattttggtttgttttgaagaagtcacttcatacctttgaactggggttaagctggttaactcccaaaattccaccagttccaatatcctatggaaaaaccccaaaaccactcaaaggatctgcgatcgctccggtgcccgtcagtgggcagagcgcacatcgcccacagtccccgagaagttggggggaggggtcggcaattgaacgggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagctgaagcttcgaggggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagtccgggcctttgtccggcgctcccttggagcctacctagactcagccggctctccacgctttgcctgaccctgcttgctcaactctacgtctttgtttcgttttctgttctgcgccgttacagatccaagctgtgaccggcgcctacgaatgccgccaccatgcacagttcagctctgctgtgctgcctcgtacttctcaccggcgtcagagcaagcccaggccagggcacccaatctgagaattcctgtacacatttccctgggaatcttcccaatatgctgcgcgacctgcgggatgccttctctcgcgttaagacttttttccagatgaaagatcagctggacaacctcttactgaaggaatcattgctcgaggattttaagggatacttaggttgccaggcgttgtccgagatgatccagttttacctggaagaggtgatgcctcaagcagaaaaccaagaccccgacatcaaagcccacgtgaatagcctaggagaaaacttgaaaacgctccgtctgcgactgcggagatgtcataggtttctaccatgtgagaacaaaagcaaggccgtcgagcaggtgaagaacgccttcaacaaacttcaggagaaagggatttataaggctatgagtgaattcgatatatttatcaattatattgaggcttacatgacaatgaagattaggaattaaaacgccatgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagagaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagtcggggtttcaccatattggccaggctggtgtccaactcctaatctcaggtgatctacccaccttggcctcccaaattgctgggattacaggcgtgaaccactgctcccttccctgtccttcagtggcatcgctcttttgcaatgctctttgggaaattatattagcctaattactaatttcctggccctcaaggtgacttgcttgaacttgccaccttcgccactagggggcagcattggtttacacagggtgaacaaacgttcaattctaaactaatattctttggtgggaaagtgtgttcattttggtttgttttgaagaagtcacttcatacctttgaactggggttaagctggttaactcccaaaattccaccagttccaatatcctatggaaaaaccccaaaaccactcaaaccagatatactcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagaaccaagcgaatgccgccaccatggctgctgaaggttccgtcgccaggcagcctgacctcttgacctgcgacgatgagccgatccatatccccggtgccatccaaccgcatggactgctgctcgccctcgccgccgacatgacgatcgttgccggcagcgacaaccttcccgaactcaccggactggcgatcggcgccctgatcggccgctctgcggccgatgtgttcgactcggaaacgcacaaccgtctgacgatcgccttggccgagcccggggcggccgtcggagcaccgatcactgtcggcttcacgatgcgaaaggacgcaggcttcatcggctcctggcatcgccatgatcagctcatcttcctggagctggagcctccccagcgggacgtcgccgagccgcaggcgttcttccgccgcaccaacagcgccatccgccgcctgcaggccgccgaaaccttggaaagcgcctgcgccgccgcggcgcaagaggtgcggaagattaccggcttcgatcgggtgatgatctatcgcttcgcctccgacttcagcggcgaagtgatcgcagaggatcggtgcgccgaggtcgagtcaaaactaggcctgcactatcctgcctcaaccgtgccggcgcaggcccgtcggctctataccatcaacccggtacggatcattcccgatatcaattatcggccggtgccggtcaccccagacctcaatccggtcaccgggcggccgattgatcttagcttcgccatcctgcgcagcgtgtcgcccgtccatctggagttcatgcgcaacataggcatgcacggcacgatgtcgatctcgattttgcgcggcgagcgactgtggggattgatcgtttgccatcaccgaacgccgtactacgtcgatctcgatggccgccaagcctgcgagctagtcgcccaggttctggcctggcagatcggcgtgatggaagagtaaaacgccatgaattcactcctcaggtgcaggctgcctatcagaaggtggtggctggtgtggccaatgccctggctcacaaataccactgagatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgcccatatgctggctgccatgaacaaaggttggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatggagatccagttcgcgcggccgcgcctgggcttacttaattaacgagagcataatattgatatgtgccaaagttgtttctgactgactaataagtataatttgtttctattatgtataggttaagctaattacttattttataatacaacatgactgtttttaaagtacaaaataagtttatttttgtaaaagagagaatgtttaaaagttttgttactttatagaagaaattttgagtttttgtttttttttaataaataaataaacataaataaattgtttgttgaatttattattagtatgtaagtgtaaatataataaaacttaatatctattcaaattaataaataaacctcgatatacagaccgataaaacacatgcgtcaattttacgcatgattatctttaacgtacgtcacaatatgattatctttctagggttaaataatagtttctaatttttttattattcagcctgctgtcgtgaataccgagctccaattcgccctatagtgagtcgtattacaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggc

SEQ ID NO: 31 - DNA, 10545 bp, synthetic construct
  source 1..10545: mol_type = other DNA
gcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctcgaaattaaccctcactaaagggaacaaaagctggtacctcgcgcgacttggtttgccattctttagcgcgcgtcgcgtcacacagcttggccacaatgtggtttttgtcaaacgaagattctatgacgtgtttaaagtttaggtcgagtaaagcgcaaatcttttttaaccctagaaagatagtctgcgtaaaattgacgcatgcattcttgaaatattgctctctctttctaaatagcgcgaatccgtcgctgtgcatttaggacatctcagtcgccgcttggagctcccgtgaggcgtgcttgtcaatgcggtaagtgtcactgattttgaactataacgaccgcgtgagtcaaaatgacgcatgattatcttttacgtgacttttaagatttaactcatacgataattatattgttatttcatgttctacttacgtgataacttattatatatatattttcttgttatagatatcatcaactttgtatagaaaagttgctcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattgcaagtttgtacaaaaaagcaggctgaatgccgccaccatggctaccgagtataaaccgacggtccgacttgcgactcgggatgacgttccacgcgccgtgaggactctggccgccgccttcgctgactacccggccacacggcacacagttgaccccgacagacacatagagcgggtcacagagttgcaagaacttttcctgacgcgggttggactcgatattggaaaagtttgggttgccgacgatggtgccgcagttgcggtgtggactactccagaaagtgtagaagctggagccgtattcgctgaaatagggccacggatggcagagctgtctggtagcaggctcgcagcgcagcagcagatggaaggccttctcgctccgcatcggcctaaggaaccggcgtggttcctggctacggtgggagtctcacctgaccatcaaggaaagggattgggaagtgctgtcgttcttccaggcgtggaagcggccgaacgagcaggcgtcccagccttcttggaaacgagcgcacccaggaatcttccattctacgagcgactgggtttcactgtgacagcagacgtagaagtccctgaagggcctcggacttggtgcatgaccagaaagcctggggcttgaaacgccatgatccagacatgataagatacattgatgagtttggacaaaccacaactagaatgcagtgaggcgcgcctggatacttctgcctgcaggggagtcgctcttttgcaatgctctttgggaaattatattagcctaattactaatttcctggccctcaaggtgacttgcttgaacttgccaccttcgccactagggggcagcattggtttacacagggtgaacaaacgttcaattctaaactaatattctttggtgggaaagtgtgttcattttggtttgttttgaagaagtcacttcatacctttgaactggggttaagctggttaactcccaaaattccaccagttccaatatcctatggaaaaaccccaaaaccactcaaagaattctaccgggtaggggaggcgcttttcccaaggcagtctggagcatgcgctttagcagcaccgctgggcacttggcgctacacaagtggcctctggcctcgcacacattccacatccaccggtaggcgccaaccggctccgttctttggtggccccttcgcgccaccttctactcctcccctagtcaggaagttcccctccgccccgcagctcgcgtcgtgcaggacgtgacaaatggaagtagcacgtctaactagtctcgtgcagatggacagcaccgctgagcaatggaagcgggtaggcctttggggcagcggccaatagcagctttgctccttcgctttctgggctcaggggcggggcgggcgcccgaaggtcctccggaggcccggcattctgcacgcttcaaaagcgcacgtctgccgcgctgttctcctcttcctcatctccgggcctttcggaatgccgccaccatgcacagttcagctctgctgtgctgcctcgtacttctcaccggcgtcagagcaagcccaggccagggcacccaatctgagaattcctgtacacatttccctgggaatcttcccaatatgctgcgcgacctgcgggatgccttctctcgcgttaagacttttttccagatgaaagatcagctggacaacctcttactgaaggaatcattgctcgaggattttaagggatacttaggttgccaggcgttgtccgagatgatccagttttacctggaagaggtgatgcctcaagcagaaaaccaagaccccgacatcaaagcccacgtgaatagcctaggagaaaacttgaaaacgctccgtctgcgactgcggagatgtcataggtttctaccatgtgagaacaaaagcaaggccgtcgagcaggtgaagaacgccttcaacaaacttcaggagaaagggatttataaggctatgagtgaattcgatatatttatcaattatattgaggcttacatgacaatgaagattaggaattaaaacgccatgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagagaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagtcggggtttcaccatattggccaggctggtgtccaactcctaatctcaggtgatctacccaccttggcctcccaaattgctgggattacaggcgtgaaccactgctcccttccctgtccttcagtggcatcgctcttttgcaatgctctttgggaaattatattagcctaattactaatttcctggccctcaaggtgacttgcttgaacttgccaccttcgccactagggggcagcattggtttacacagggtgaacaaacgttcaattctaaactaatattctttggtgggaaagtgtgttcattttggtttgttttgaagaagtcacttcatacctttgaactggggttaagctggttaactcccaaaattccaccagttccaatatcctatggaaaaaccccaaaaccactcaaaccagatatactcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagaaccaagcgaatgccgccaccatggctgctgaaggttccgtcgccaggcagcctgacctcttgacctgcgacgatgagccgatccatatccccggtgccatccaaccgcatggactgctgctcgccctcgccgccgacatgacgatcgttgccggcagcgacaaccttcccgaactcaccggactggcgatcggcgccctgatcggccgctctgcggccgatgtgttcgactcggaaacgcacaaccgtctgacgatcgccttggccgagcccggggcggccgtcggagcaccgatcactgtcggcttcacgatgcgaaaggacgcaggcttcatcggctcctggcatcgccatgatcagctcatcttcctggagctggagcctccccagcgggacgtcgccgagccgcaggcgttcttccgccgcaccaacagcgccatccgccgcctgcaggccgccgaaaccttggaaagcgcctgcgccgccgcggcgcaagaggtgcggaagattaccggcttcgatcgggtgatgatctatcgcttcgcctccgacttcagcggcgaagtgatcgcagaggatcggtgcgccgaggtcgagtcaaaactaggcctgcactatcctgcctcaaccgtgccggcgcaggcccgtcggctctataccatcaacccggtacggatcattcccgatatcaattatcggccggtgccggtcaccccagacctcaatccggtcaccgggcggccgattgatcttagcttcgccatcctgcgcagcgtgtcgcccgtccatctggagttcatgcgcaacataggcatgcacggcacgatgtcgatctcgattttgcgcggcgagcgactgtggggattgatcgtttgccatcaccgaacgccgtactacgtcgatctcgatggccgccaagcctgcgagctagtcgcccaggttctggcctggcagatcggcgtgatggaagagtaaaacgccatgaattcactcctcaggtgcaggctgcctatcagaaggtggtggctggtgtggccaatgccctggctcacaaataccactgagatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgcccatatgctggctgccatgaacaaaggttggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatggagatccagttcgcgcggccgcgcctgggcttacttaattaacgagagcataatattgatatgtgccaaagttgtttctgactgactaataagtataatttgtttctattatgtataggttaagctaattacttattttataatacaacatgactgtttttaaagtacaaaataagtttatttttgtaaaagagagaatgtttaaaagttttgttactttatagaagaaattttgagtttttgtttttttttaataaataaataaacataaataaattgtttgttgaatttattattagtatgtaagtgtaaatataataaaacttaatatctattcaaattaataaataaacctcgatatacagaccgataaaacacatgcgtcaattttacgcatgattatctttaacgtacgtcacaatatgattatctttctagggttaaataatagtttctaatttttttattattcagcctgctgtcgtgaataccgagctccaattcgccctatagtgagtcgtattacaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggc

SEQ ID NO: 32 - DNA, 8421 bp, synthetic construct
  source 1..8421: mol_type = other DNA
tagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcagcacgtgttgacaattaatcatcggcatagtatatcggcatagtataatacgacaaggtgaggaactaaaccatggccaagttgaccagtgccgttccggtgctcaccgcgcgcgacgtcgccggagcggtcgagttctggaccgaccggctcgggttctcccgggacttcgtggaggacgacttcgccggtgtggtccgggacgacgtgaccctgttcatcagcgcggtccaggaccaggtggtgccggacaacaccctggcctgggtgtgggtgcgcggcctggacgagctgtacgccgagtggtcggaggtcgtgtccacgaacttccgggacgcctccgggccggccatgaccgagatcggcgagcagccgtgggggcgggagttcgccctgcgcgacccggccggcaactgcgtgcacttcgtggccgaggagcaggactgacacgtgctacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtcgacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagcgcgttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatcggcactgcgtgcgccaattctgcagacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttggttaattaaggtaccggagatgtcttccgttgctagccctgcacgcttctttacagctagctcagtcctaggtattatgctagctactcgacctagtgaaagaggagaaatactaaatggtgagtaaaggagaagaaaacaacttagctatcattaaagagttcatgcgcttcaaagttcacatggagggttctgttaacggtcacgagttcgagatcgaaggcgaaggcgagggccgtccgtatgaaggcacccagaccgccaaactgaaagtgactaaaggcggcccgctgccttttgcgtgggacatcctgagcccgcaatttatgtacggttctaaagcgtatgttaaacacccagcggatatcccggactatctgaagctgtcttttccggaaggtttcaactgggaacgcgtaatgaattttgaagatggtggtgtcgtgaccgtcactcaggactcctcccttcaggatggcgagttcatctataaagttaaactgcgtggtactaattttccatctgatggcccggtgatgcagtgtaggacgatgggttgggaggcgtctaccgaacgcatgtatccggaagatggtgcgctgaaaggcgaaattaaacagcgcctgaaactgaaagatggcggccattatgacgctgaagtgaaaaccacgtacaaagccaagaaacctgtgcagctgcctggcgcgtacaatgtggatattaaactggacatcttatctcataatgaagattatacgatcgtagagcaatatgagcgcgcggagggtcgtcattctaccggtggcatggatgaactatacaaataatggcctcgcgtacgcaaagacgaacaataagacgctgaaaagcgtcttttttcgttttggtccgctgttcctgaagacatggcataagtcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtctacgccttcgccctcagacgagtcggatctccctttgggccgcctccccgcgtcgactttaagaccaatgacttacaaggcagctgtagatcttagccactttttaaaagaaaaggggggactggaagggctaattcactcccaacgaagtcaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagagaa

SEQ ID NO: 33 - DNA, 8432 bp, synthetic construct
  source 1..8432: mol_type = other DNA
tagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcagcacgtgttgacaattaatcatcggcatagtatatcggcatagtataatacgacaaggtgaggaactaaaccatggccaagttgaccagtgccgttccggtgctcaccgcgcgcgacgtcgccggagcggtcgagttctggaccgaccggctcgggttctcccgggacttcgtggaggacgacttcgccggtgtggtccgggacgacgtgaccctgttcatcagcgcggtccaggaccaggtggtgccggacaacaccctggcctgggtgtgggtgcgcggcctggacgagctgtacgccgagtggtcggaggtcgtgtccacgaacttccgggacgcctccgggccggccatgaccgagatcggagagcagccatggggacgtgagttcgccctgcgtgacccagccggaaactgcgtgcacttcgtggccgaagaacaggactgacacgtgctacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtcgacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagcgcgttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatcggcactgcgtgcgccaattctgcagacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttggttaattaaggtaccggagtgagaccggagatgtcttccgttgctagccctgcacgcttctttacagctagctcagtcctaggtattatgctagctactcgacctagtgaaagaggagaaatactaaatggtgagtaaaggagaagaaaacaacttagctatcattaaagagttcatgcgcttcaaagttcacatggagggttctgttaacggtcacgagttcgagatcgaaggcgaaggcgagggccgtccgtatgaaggcacccagaccgccaaactgaaagtgactaaaggcggcccgctgccttttgcgtgggacatcctgagcccgcaatttatgtacggttctaaagcgtatgttaaacacccagcggatatcccggactatctgaagctgtcttttccggaaggtttcaactgggaacgcgtaatgaattttgaagatggtggtgtcgtgaccgtcactcaggactcctcccttcaggatggcgagttcatctataaagttaaactgcgtggtactaattttccatctgatggcccggtgatgcagtgtaggacgatgggttgggaggcgtctaccgaacgcatgtatccggaagatggtgcgctgaaaggcgaaattaaacagcgcctgaaactgaaagatggcggccattatgacgctgaagtgaaaaccacgtacaaagccaagaaacctgtgcagctgcctggcgcgtacaatgtggatattaaactggacatcttatctcataatgaagattatacgatcgtagagcaatatgagcgcgcggagggtcgtcattctaccggtggcatggatgaactatacaaataatggcctcgcgtacgcaaagacgaacaataagacgctgaaaagcgtcttttttcgttttggtccgctgttcctgaagacattcgctaagtcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtctacgccttcgccctcagacgagtcggatctccctttgggccgcctccccgcgtcgactttaagaccaatgacttacaaggcagctgtagatcttagccactttttaaaagaaaaggggggactggaagggctaattcactcccaacgaagtcaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagagaa

SEQ ID NO: 34 - DNA, 11641 bp, synthetic construct
  source 1..11641: mol_type = other DNA
ataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatcggcactgcgtgcgccaattctgcagacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttggttaattaaggtaccggagcgttttacttgtacagctcgtccatgccgagagtgatcccggcggcggtcacgaactccagcaggaccatgtgatcgcgcttctcgttggggtctttgctcagggcggactgggtgctcaggtagtggttgtcgggcagcagcacggggccgtcgccgatgggggtgttctgctggtagtggtcggcgagctgcacgctgccgtcctcgatgttgtggcggatcttgaagttcaccttgatgccgttcttctgcttgtcggccatgatatagacgttgtggctgttgtagttgtactccagcttgtgccccaggatgttgccgtcctccttgaagtcgatgcccttcagctcgatgcggttcaccagggtgtcgccctcgaacttcacctcggcgcgggtcttgtagttgccgtcgtccttgaagaagatggtgcgctcctggacgtagccttcgggcatggcggacttgaagaagtcgtgctgcttcatgtggtcggggtagcggctgaagcactgcacgccgtaggtcagggtggtcacgagggtgggccagggcacgggcagcttgccggtggtgcagatgaacttcagggtcagcttgccgtaggtggcatcgccctcgccctcgccggacacgctgaacttgtggccgtttacgtcgccgtccagctcgaccaggatgggcaccaccccggtgaacagctcctcgcccttgctcaccatagccatggtggcggcattcgcttggttctccctatagtgagtcgtattaatttcgataagccagtaagcagtgggttctctagttagccagagagctctgcttatatagacctcccaccgtacacgcctaccgcccatttgcgtcaatggggcggagttgttacgacattttggaaagtcccgttgattttggtgccaaaacaaactcccattgacgtcaatggggtggagacttggaaatccccgtgagtcaaaccgctatccacgcccattgatgtactgccaaaaccgcatcaccatggtaatagcgatgactaatacgtagatgtactgccaagtaggaaagtcccataaggtcatgtactgggcataatgccaggcgggccatttaccgtcattgacgtcaatagggggcgtacttggcatatgatacacttgatgtactgccaagtgggcagtttaccgtaaatactccacccattgacgtcaatggaaagtccctattggcgttactatgggaacatacgtcattattgacgtcaatgggcgggggtcgttgggcggtcagccaggcgggccatttaccgtaagttatgtaacgcggaactccatatatgggctatgaactaatgaccccgtaattgattactattaataactagtcaataatcaatgtcaacgagtatatctggtttgagtggttttggggtttttccataggatattggaactggtggaattttgggagttaaccagcttaaccccagttcaaaggtatgaagtgacttcttcaaaacaaaccaaaatgaacacactttcccaccaaagaatattagtttagaattgaacgtttgttcaccctgtgtaaaccaatgctgccccctagtggcgaaggtggcaagttcaagcaagtcaccttgagggccaggaaattagtaattaggctaatataatttcccaaagagcattgcaaaagagcgaggcatcgccaaaatcaactttgtatagaaaagttgctcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattgcaagtttgtacaaaaaagcaggctgaatgccgccaccatggctaccgagtataaaccgacggtccgacttgcgactcgggatgacgttccacgcgccgtgaggactctggccgccgccttcgctgactacccggccacacggcacacagttgaccccgacagacacatagagcgggtcacagagttgcaagaacttttcctgacgcgggttggactcgatattggaaaagtttgggttgccgacgatggtgccgcagttgcggtgtggactactccagaaagtgtagaagctggagccgtattcgctgaaatagggccacggatggcagagctgtctggtagcaggctcgcagcgcagcagcagatggaaggccttctcgctccgcatcggcctaaggaaccggcgtggttcctggctacggtgggagtctcacctgaccatcaaggaaagggattgggaagtgctgtcgttcttccaggcgtggaagcggccgaacgagcaggcgtcccagccttcttggaaacgagcgcacccaggaatcttccattctacgagcgactgggtttcactgtgacagcagacgtagaagtccctgaagggcctcggacttggtgcatgaccagaaagcctggggcttgaaacgtcgcaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtctacgccttcgccctcagacgagtcggatctccctttgggccgcctccccgcgtcgactttaagaccaatgacttacaaggcagctgtagatcttagccactttttaaaagaaaaggggggactggaagggctaattcactcccaacgaagtcaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagagaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcagcacgtgttgacaattaatcatcggcatagtatatcggcatagtataatacgacaaggtgaggaactaaaccatggccaagttgaccagtgccgttccggtgctcaccgcgcgcgacgtcgccggagcggtcgagttctggaccgaccggctcgggttctcccgggacttcgtggaggacgacttcgccggtgtggtccgggacgacgtgaccctgttcatcagcgcggtccaggaccaggtggtgccggacaacaccctggcctgggtgtgggtgcgcggcctggacgagctgtacgccgagtggtcggaggtcgtgtccacgaacttccgggacgcctccgggccggccatgaccgagatcggcgagcagccgtgggggcgggagttcgccctgcgcgacccggccggcaactgcgtgcacttcgtggccgaggagcaggactgacacgtgctacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtcgacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagcgcgttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagag

SEQ ID NO: 35 - DNA, 9257 bp, synthetic construct
  source 1..9257: mol_type = other DNA
tagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcagcacgtgttgacaattaatcatcggcatagtatatcggcatagtataatacgacaaggtgaggaactaaaccatggccaagttgaccagtgccgttccggtgctcaccgcgcgcgacgtcgccggagcggtcgagttctggaccgaccggctcgggttctcccgggacttcgtggaggacgacttcgccggtgtggtccgggacgacgtgaccctgttcatcagcgcggtccaggaccaggtggtgccggacaacaccctggcctgggtgtgggtgcgcggcctggacgagctgtacgccgagtggtcggaggtcgtgtccacgaacttccgggacgcctccgggccggccatgaccgagatcggcgagcagccgtgggggcgggagttcgccctgcgcgacccggccggcaactgcgtgcacttcgtggccgaggagcaggactgacacgtgctacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtcgacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagcgcgttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatcggcactgcgtgcgccaattctgcagacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttggttaattaaggtaccggagtgagaccggagcgttttacttgtacagctcgtccatgccgagagtgatcccggcggcggtcacgaactccagcaggaccatgtgatcgcgcttctcgttggggtctttgctcagggcggactgggtgctcaggtagtggttgtcgggcagcagcacggggccgtcgccgatgggggtgttctgctggtagtggtcggcgagctgcacgctgccgtcctcgatgttgtggcggatcttgaagttcaccttgatgccgttcttctgcttgtcggccatgatatagacgttgtggctgttgtagttgtactccagcttgtgccccaggatgttgccgtcctccttgaagtcgatgcccttcagctcgatgcggttcaccagggtgtcgccctcgaacttcacctcggcgcgggtcttgtagttgccgtcgtccttgaagaagatggtgcgctcctggacgtagccttcgggcatggcggacttgaagaagtcgtgctgcttcatgtggtcggggtagcggctgaagcactgcacgccgtaggtcagggtggtcacgagggtgggccagggcacgggcagcttgccggtggtgcagatgaacttcagggtcagcttgccgtaggtggcatcgccctcgccctcgccggacacgctgaacttgtggccgtttacgtcgccgtccagctcgaccaggatgggcaccaccccggtgaacagctcctcgcccttgctcacagccatggtggcggcattcgcttggttctccctatagtgagtcgtattaatttcgataagccagtaagcagtgggttctctagttagccagagagctctgcttatatagacctcccaccgtacacgcctaccgcccatttgcgtcaatggggcggagttgttacgacattttggaaagtcccgttgattttggtgccaaaacaaactcccattgacgtcaatggggtggagacttggaaatccccgtgagtcaaaccgctatccacgcccattgatgtactgccaaaaccgcatcaccatggtaatagcgatgactaatacgtagatgtactgccaagtaggaaagtcccataaggtcatgtactgggcataatgccaggcgggccatttaccgtcattgacgtcaatagggggcgtacttggcatatgatacacttgatgtactgccaagtgggcagtttaccgtaaatactccacccattgacgtcaatggaaagtccctattggcgttactatgggaacatacgtcattattgacgtcaatgggcgggggtcgttgggcggtcagccaggcgggccatttaccgtaagttatgtaacgcggaactccatatatgggctatgaactaatgaccccgtaattgattactattaataactagtcaataatcaatgtcaacgagtatatctggtttgagtggttttggggtttttccataggatattggaactggtggaattttgggagttaaccagcttaaccccagttcaaaggtatgaagtgacttcttcaaaacaaaccaaaatgaacacactttcccaccaaagaatattagtttagaattgaacgtttgttcaccctgtgtaaaccaatgctgccccctagtggcgaaggtggcaagttcaagcaagtcaccttgagggccaggaaattagtaattaggctaatataatttcccaaagagcattgcaaaagagcgaggcaggtctctggcataagtcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtctacgccttcgccctcagacgagtcggatctccctttgggccgcctccccgcgtcgactttaagaccaatgacttacaaggcagctgtagatcttagccactttttaaaagaaaaggggggactggaagggctaattcactcccaacgaagtcaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagagaa

SEQ ID NO: 36 - DNA, 10405 bp, synthetic construct
  source 1..10405: mol_type = other DNA
tagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcagcacgtgttgacaattaatcatcggcatagtatatcggcatagtataatacgacaaggtgaggaactaaaccatggccaagttgaccagtgccgttccggtgctcaccgcgcgcgacgtcgccggagcggtcgagttctggaccgaccggctcgggttctcccgggacttcgtggaggacgacttcgccggtgtggtccgggacgacgtgaccctgttcatcagcgcggtccaggaccaggtggtgccggacaacaccctggcctgggtgtgggtgcgcggcctggacgagctgtacgccgagtggtcggaggtcgtgtccacgaacttccgggacgcctccgggccggccatgaccgagatcggcgagcagccgtgggggcgggagttcgccctgcgcgacccggccggcaactgcgtgcacttcgtggccgaggagcaggactgacacgtgctacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtcgacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagcgcgttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatcggcactgcgtgcgccaattctgcagacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttggttaattaaggtaccggagcgttttacttgtacagctcgtccatgccgagagtgatcccggcggcggtcacgaactccagcaggaccatgtgatcgcgcttctcgttggggtctttgctcagggcggactgggtgctcaggtagtggttgtcgggcagcagcacggggccgtcgccgatgggggtgttctgctggtagtggtcggcgagctgcacgctgccgtcctcgatgttgtggcggatcttgaagttcaccttgatgccgttcttctgcttgtcggccatgatatagacgttgtggctgttgtagttgtactccagcttgtgccccaggatgttgccgtcctccttgaagtcgatgcccttcagctcgatgcggttcaccagggtgtcgccctcgaacttcacctcggcgcgggtcttgtagttgccgtcgtccttgaagaagatggtgcgctcctggacgtagccttcgggcatggcggacttgaagaagtcgtgctgcttcatgtggtcggggtagcggctgaagcactgcacgccgtaggtcagggtggtcacgagggtgggccagggcacgggcagcttgccggtggtgcagatgaacttcagggtcagcttgccgtaggtggcatcgccctcgccctcgccggacacgctgaacttgtggccgtttacgtcgccgtccagctcgaccaggatgggcaccaccccggtgaacagctcctcgcccttgctcaccatagccatggtggcggcattcgcttggttctccctatagtgagtcgtattaatttcgataagccagtaagcagtgggttctctagttagccagagagctctgcttatatagacctcccaccgtacacgcctaccgcccatttgcgtcaatggggcggagttgttacgacattttggaaagtcccgttgattttggtgccaaaacaaactcccattgacgtcaatggggtggagacttggaaatccccgtgagtcaaaccgctatccacgcccattgatgtactgccaaaaccgcatcaccatggtaatagcgatgactaatacgtagatgtactgccaagtaggaaagtcccataaggtcatgtactgggcataatgccaggcgggccatttaccgtcattgacgtcaatagggggcgtacttggcatatgatacacttgatgtactgccaagtgggcagtttaccgtaaatactccacccattgacgtcaatggaaagtccctattggcgttactatgggaacatacgtcattattgacgtcaatgggcgggggtcgttgggcggtcagccaggcgggccatttaccgtaagttatgtaacgcggaactccatatatgggctatgaactaatgaccccgtaattgattactattaataactagtcaataatcaatgtcaacgagtatatctggtttgagtggttttggggtttttccataggatattggaactggtggaattttgggagttaaccagcttaaccccagttcaaaggtatgaagtgacttcttcaaaacaaaccaaaatgaacacactttcccaccaaagaatattagtttagaattgaacgtttgttcaccctgtgtaaaccaatgctgccccctagtggcgaaggtggcaagttcaagcaagtcaccttgagggccaggaaattagtaattaggctaatataatttcccaaagagcattgcaaaagagcgaggcatcgccaaaggatctgcgatcgctccggtgcccgtcagtgggcagagcgcacatcgcccacagtccccgagaagttggggggaggggtcggcaattgaacgggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagctgaagcttcgaggggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagtccgggcctttgtccggcgctcccttggagcctacctagactcagccggctctccacgctttgcctgaccctgcttgctcaactctacgtctttgtttcgttttctgttctgcgccgttacagatccaagctgtgaccggcgcctacgaatgccgccaccatggctaccgagtataaaccgacggtccgacttgcgactcgggatgacgttccacgcgccgtgaggactctggccgccgccttcgctgactacccggccacacggcacacagttgaccccgacagacacatagagcgggtcacagagttgcaagaacttttcctgacgcgggttggactcgatattggaaaagtttgggttgccgacgatggtgccgcagttgcggtgtggactactccagaaagtgtagaagctggagccgtattcgctgaaatagggccacggatggcagagctgtctggtagcaggctcgcagcgcagcagcagatggaaggccttctcgctccgcatcggcctaaggaaccggcgtggttcctggctacggtgggagtctcacctgaccatcaaggaaagggattgggaagtgctgtcgttcttccaggcgtggaagcggccgaacgagcaggcgtcccagccttcttggaaacgagcgcacccaggaatcttccattctacgagcgactgggtttcactgtgacagcagacgtagaagtccctgaagggcctcggacttggtgcatgaccagaaagcctggggcttgaaacgtcgcaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtctacgccttcgccctcagacgagtcggatctccctttgggccgcctccccgcgtcgactttaagaccaatgacttacaaggcagctgtagatcttagccactttttaaaagaaaaggggggactggaagggctaattcactcccaacgaagtcaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagagaa

SEQ ID NO: 37 - DNA, 13299 bp, synthetic construct
  source 1..13299: mol_type = other DNA
ttttatctctcattcgaaaattcccaacatcgggattatataactgaaaagttttggcgtaatgcgttggtcgcaggaaccgtacccgtagtcctcggaccaccccgcgcaacttacgaggcttttgttcccgcagacgcattcgtacacgtggacgatttcggttccgctcgggaactcgctgcatttctgacaggtatgaacgaatctaggtatcagcggttcttcgcgtggcgggatcggcttcgggtcaggttgtttacggattggagagagagattttgcgcaatatgcgataggtatcctcatctgcctagatcccaggtgtacgaagatttggaaggctggttccaagcatagaacgccatgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagagaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagtcggggtttcaccatattggccaggctggtgtccaactcctaatctcaggtgatctacccaccttggcctcccaaattgctgggattacaggcgtgaaccactgctcccttccctgtccttcagtggcatcgctcttttgcaatgctctttgggaaattatattagcctaattactaatttcctggccctcaaggtgacttgcttgaacttgccaccttcgccactagggggcagcattggtttacacagggtgaacaaacgttcaattctaaactaatattctttggtgggaaagtgtgttcattttggtttgttttgaagaagtcacttcatacctttgaactggggttaagctggttaactcccaaaattccaccagttccaatatcctatggaaaaaccccaaaaccactcaaaggatctgcgatcgctccggtgcccgtcagtgggcagagcgcacatcgcccacagtccccgagaagttggggggaggggtcggcaattgaacgggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagctgaagcttcgaggggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagtccgggcctttgtccggcgctcccttggagcctacctagactcagccggctctccacgctttgcctgaccctgcttgctcaactctacgtctttgtttcgttttctgttctgcgccgttacagatccaagctgtgaccggcgcctacgaatgccgccaccatgccccttcagctgttgttgcttttgatactgctgggacctggaaactcacttcagttgtgggacacatgggccgatgaggcggagaaggccctgggtccccttctcgcaagagacagacggcaagcgactgagtatgaatacctcgattatgatttcctccccgagactgagcctcctgagatgttgcgcaacagtacagacacaacgccattgactggccccggaactccagagagcaccacggttgagcctgctgcccggagaagtactggattggatgcgggcggcgctgtgactgaattgaccactgagctggccaatatggggaatctgtccacagacagcgcggccatggagatacaaacgacacagcctgcggccaccgaggcccagactacacagcctgtaccgacagaggcccaaactacaccattggcagctacggaagctcaaacaactcgattgaccgctaccgaggcacagactacccctctcgctgcgactgaagcccaaacgacaccacctgcagcaacagaagcacaaactacacagcccacagggttggaggcgcaaacaactgcgcctgctgccatggaagctcaaacaacggcaccggccgcaatggaggcacagactactcctcctgcagctatggaggcccaaacgacacagacgactgccatggaggcacagacaactgcaccagaggccacggaggcacaaacaacacaaccaactgcaacagaggcgcagacgacgcccctcgcagcaatggaagctctttccacggaacccagcgcaaccgaagcactctctatggagcctacaaccaaacggggcctttttattccattctctgtgtcatccgtgactcataaggggatacctatggctgcgtctaatctctcagtcaattatccagttggtgcgccagatcacatttccgtgaaacagtgtctccttgccatattgatacttgccttggtcgcgactatttttttcgtgtgtaccgtagtgctcgcggttcgcctctctcggaagggtcacatgtaccctgttaggaattattctcccactgaaatggtgtgcatcagcagtctgctgcctgatggtggcgagggaccatccgctacagccaacggaggactctctaaggctaagtctcctggactgacccctgagccccgcgaggatagggaaggtgacgatctcaccctccactcctttctgccatagaacgccatgaattcactcctcaggtgcaggctgcctatcagaaggtggtggctggtgtggccaatgccctggctcacaaataccactgagatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgcccatatgctggctgccatgaacaaaggttggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatggagatcagttcgctcgctcttttgcaatgctctttgggaaattatattagcctaattactaatttcctggccctcaaggtgacttgcttgaacttgccaccttcgccactagggggcagcattggtttacacagggtgaacaaacgttcaattctaaactaatattctttggtgggaaagtgtgttcattttggtttgttttgaagaagtcacttcatacctttgaactggggttaagctggttaactcccaaaattccaccagttccaatatcctatggaaaaaccccaaaaccactcaaaggatctgccatcgctccggtgcccgtcagtgggcagagcgcacatcgcccacagtccccgagaagttggggggaggggtcggcaattgaacgggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagctgaagcttcgaggggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagtccgggcctttgtccggcgctcccttggagcctacctagactcagccggctctccacgctttgcctgaccctgcttgctcaactctacgtctttgtttcgttttctgttctgcgccgttacagatccaagctgtgaccggcgcctacgaatgccgccaccatggctgctgaaggttccgtcgccaggcagcctgacctcttgacctgcgacgatgagccgatccatatccccggtgccatccaaccgcatggactgctgctcgccctcgccgccgacatgacgatcgttgccggcagcgacaaccttcccgaactcaccggactggcgatcggcgccctgatcggccgctctgcggccgatgtgttcgactcggaaacgcacaaccgtctgacgatcgccttggccgagcccggggcggccgtcggagcaccgatcactgtcggcttcacgatgcgaaaggacgcaggcttcatcggctcctggcatcgccatgatcagctcatcttcctggagctggagcctccccagcgggacgtcgccgagccgcaggcgttcttccgccgcaccaacagcgccatccgccgcctgcaggccgccgaaaccttggaaagcgcctgcgccgccgcggcgcaagaggtgcggaagattaccggcttcgatcgggtgatgatctatcgcttcgcctccgacttcagcggcgaagtgatcgcagaggatcggtgcgccgaggtcgagtcaaaactaggcctgcactatcctgcctcaaccgtgccggcgcaggcccgtcggctctataccatcaacccggtacggatcattcccgatatcaattatcggccggtgccggtcaccccagacctcaatccggtcaccgggcggccgattgatcttagcttcgccatcctgcgcagcgtgtcgcccgtccatctggagttcatgcgcaacataggcatgcacggcacgatgtcgatctcgattttgcgcggcgagcgactgtggggattgatcgtttgccatcaccgaacgccgtactacgtcgatctcgatggccgccaagcctgcgagctagtcgcccaggttctggcctggcagatcggcgtgatggaagagtaaaacgbbabbabbabbabbabbaccatctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaaggcaatagcaggcatgctggggatgcggtgggctctatggcagtcagtgcggccgcgcctgggcttacttaattaacgagagcataatattgatatgtgccaaagttgtttctgactgactaataagtataatttgtttctattatgtataggttaagctaattacttattttataatacaacatgactgtttttaaagtacaaaataagtttatttttgtaaaagagagaatgtttaaaagttttgttactttatagaagaaattttgagtttttgtttttttttaataaataaataaacataaataaattgtttgttgaatttattattagtatgtaagtgtaaatataataaaacttaatatctattcaaattaataaataaacctcgatatacagaccgataaaacacatgcgtcaattttacgcatgattatctttaacgtacgtcacaatatgattatctttctagggttaaataatagtttctaatttttttattattcagcctgctgtcgtgaataccgagctccaattcgccctatagtgagtcgtattacaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctcgaaattaaccctcactaaagggaacaaaagctggtacctcgcgcgacttggtttgccattctttagcgcgcgtcgcgtcacacagcttggccacaatgtggtttttgtcaaacgaagattctatgacgtgtttaaagtttaggtcgagtaaagcgcaaatcttttttaaccctagaaagatagtctgcgtaaaattgacgcatgcattcttgaaatattgctctctctttctaaatagcgcgaatccgtcgctgtgcatttaggacatctcagtcgccgcttggagctcccgtgaggcgtgcttgtcaatgcggtaagtgtcactgattttgaactataacgaccgcgtgagtcaaaatgacgcatgattatcttttacgtgacttttaagatttaactcatacgataattatattgttatttcatgttctacttacgtgataacttattatatatatattttcttgttatagatatcatcaactttgtatagaaaagttgctcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattgcaagtttgtacaaaaaagcaggctgaatgccgccaccatggctaccgagtataaaccgacggtccgacttgcgactcgggatgacgttccacgcgccgtgaggactctggccgccgccttcgctgactacccggccacacggcacacagttgaccccgacagacacatagagcgggtcacagagttgcaagaacttttcctgacgcgggttggactcgatattggaaaagtttgggttgccgacgatggtgccgcagttgcggtgtggactactccagaaagtgtagaagctggagccgtattcgctgaaatagggccacggatggcagagctgtctggtagcaggctcgcagcgcagcagcagatggaaggccttctcgctccgcatcggcctaaggaaccggcgtggttcctggctacggtgggagtctcacctgaccatcaaggaaagggattgggaagtgctgtcgttcttccaggcgtggaagcggccgaacgagcaggcgtcccagccttcttggaaacgagcgcacccaggaatcttccattctacgagcgactgggtttcactgtgacagcagacgtagaagtccctgaagggcctcggacttggtgcatgaccagaaagcctggggcttgaaacgccatgatccagacatgataagatacattgatgagtttggacaaaccacaactagaatgcagtgaggcgcgcctggatacttctgcctgcaggggagtcgctcttttgcaatgctctttgggaaattatattagcctaattactaatttcctggccctcaaggtgacttgcttgaacttgccaccttcgccactagggggcagcattggtttacacagggtgaacaaacgttcaattctaaactaatattctttggtgggaaagtgtgttcattttggtttgttttgaagaagtcacttcatacctttgaactggggttaagctggttaactcccaaaattccaccagttccaatatcctatggaaaaaccccaaaaccactcaaaggggttggggttgcgccttttccaaggcagccctgggtttgcgcagggacgcggctgctctgggcgtggttccgggaaacgcagcggcgccgaccctgggtctggcacattcttcacgtccgttcgcagcgtcacccggatcttcgccgctacccttgtgggccccccggcgacgcttcctgctccgcccctaagtcgggaaggttccttgcggttcgcggcgtgccggacgtgacaaacggaagccgcacgtctgactagtaccctcgcagacggacagcgccagggagcaatggcagcgcgccgaccgcgatgggctgtggccaatagcggctgctcagcagggcgcgccgagagcagcggccgggaaggggcggtgcgggaggcggggtgtggggcggtagtgtgggccctgttcctgcccgcgcggtgttccgcattctgcaagcctccggagcgcacgtcggcagtcggctccctcgttgaccgaatcaccgacctctctccccaggaatgccgccaccatgaataacgccggccacggaccaactagacgcttgaggggactcggagttttggctggagtcgcactcctggccgctctgtggctgctttggctccttggaagcgcgccaaggggcacacctgcaccacaacctaccataacaattctggtgtggcattggccatttaccgatcaaccacccgaattgccgtcagatacatgtaccaggtatgggattgcacggtgtcatctcagcgcgaatcgctcattgttggcttcagcagatgcggtagtgtttcatcatagagaacttcaaactcgcaggagccaccttcctttggcacaaaggccgcggggtcaaccgtgggtttgggcatcaatggaaagtccctcacatactcatgggctgtcacatctgcggggaatctttaattgggtactttcctatcgacgggatagcgatattttcgtcccatacggacgattggaacctcattggggtccttctccccctctccctgcaaaatccagagttgcagcttgggtcgtttccaattttcaagaacgccaacttagggctcgcctctatagacaactcgctccccaccttcgtgttgacgttttcggtcgagctaacgggcgcccgttgtgtgcgtcatgtctcgtacctacggttgctcaatatcgg

SEQ ID NO: 38 - DNA, 15781 bp, synthetic construct
  source 1..15781: mol_type = other DNA
tgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctcgaaattaaccctcactaaagggaacaaaagctggtacctcgcgcgacttggtttgccattctttagcgcgcgtcgcgtcacacagcttggccacaatgtggtttttgtcaaacgaagattctatgacgtgtttaaagtttaggtcgagtaaagcgcaaatcttttttaaccctagaaagatagtctgcgtaaaattgacgcatgcattcttgaaatattgctctctctttctaaatagcgcgaatccgtcgctgtgcatttaggacatctcagtcgccgcttggagctcccgtgaggcgtgcttgtcaatgcggtaagtgtcactgattttgaactataacgaccgcgtgagtcaaaatgacgcatgattatcttttacgtgacttttaagatttaactcatacgataattatattgttatttcatgttctacttacgtgataacttattatatatatattttcttgttatagatatcatcaactttgtatagaaaagttgctcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattgcaagtttgtacaaaaaagcaggctgaatgccgccaccatggctaccgagtataaaccgacggtccgacttgcgactcgggatgacgttccacgcgccgtgaggactctggccgccgccttcgctgactacccggccacacggcacacagttgaccccgacagacacatagagcgggtcacagagttgcaagaacttttcctgacgcgggttggactcgatattggaaaagtttgggttgccgacgatggtgccgcagttgcggtgtggactactccagaaagtgtagaagctggagccgtattcgctgaaatagggccacggatggcagagctgtctggtagcaggctcgcagcgcagcagcagatggaaggccttctcgctccgcatcggcctaaggaaccggcgtggttcctggctacggtgggagtctcacctgaccatcaaggaaagggattgggaagtgctgtcgttcttccaggcgtggaagcggccgaacgagcaggcgtcccagccttcttggaaacgagcgcacccaggaatcttccattctacgagcgactgggtttcactgtgacagcagacgtagaagtccctgaagggcctcggacttggtgcatgaccagaaagcctggggcttgaaacgccatgatccagacatgataagatacattgatgagtttggacaaaccacaactagaatgcagtgaggcgcgcctggatacttctgcctgcaggggagtcgctcttttgcaatgctctttgggaaattatattagcctaattactaatttcctggccctcaaggtgacttgcttgaacttgccaccttcgccactagggggcagcattggtttacacagggtgaacaaacgttcaattctaaactaatattctttggtgggaaagtgtgttcattttggtttgttttgaagaagtcacttcatacctttgaactggggttaagctggttaactcccaaaattccaccagttccaatatcctatggaaaaaccccaaaaccactcaaaggggttggggttgcgccttttccaaggcagccctgggtttgcgcagggacgcggctgctctgggcgtggttccgggaaacgcagcggcgccgaccctgggtctggcacattcttcacgtccgttcgcagcgtcacccggatcttcgccgctacccttgtgggccccccggcgacgcttcctgctccgcccctaagtcgggaaggttccttgcggttcgcggcgtgccggacgtgacaaacggaagccgcacgtctgactagtaccctcgcagacggacagcgccagggagcaatggcagcgcgccgaccgcgatgggctgtggccaatagcggctgctcagcagggcgcgccgagagcagcggccgggaaggggcggtgcgggaggcggggtgtggggcggtagtgtgggccctgttcctgcccgcgcggtgttccgcattctgcaagcctccggagcgcacgtcggcagtcggctccctcgttgaccgaatcaccgacctctctccccaggaatgccgccaccatgaataacgccggccacggaccaactagacgcttgaggggactcggagttttggctggagtcgcactcctggccgctctgtggctgctttggctccttggaagcgcgccaaggggcacacctgcaccacaacctaccataacaattctggtgtggcattggccatttaccgatcaaccacccgaattgccgtcagatacatgtaccaggtatgggattgcacggtgtcatctcagcgcgaatcgctcattgttggcttcagcagatgcggtagtgtttcatcatagagaacttcaaactcgcaggagccaccttcctttggcacaaaggccgcggggtcaaccgtgggtttgggcatcaatggaaagtccctcacatactcatgggctgtcacatctgcggggaatctttaattgggtactttcctatcgacgggatagcgatattttcgtcccatacggacgattggaacctcattggggtccttctccccctctccctgcaaaatccagagttgcagcttgggtcgtttccaattttcaagaacgccaacttagggctcgcctctatagacaactcgctccccaccttcgtgttgacgttttcggtcgagctaacgggcgcccgttgtgtgcgtcatgtctcgtacctacggttgctcaatatcggttttatctctcattcgaaaattcccaacatcgggattatataactgaaaagttttggcgtaatgcgttggtcgcaggaaccgtacccgtagtcctcggaccaccccgcgcaacttacgaggcttttgttcccgcagacgcattcgtacacgtggacgatttcggttccgctcgggaactcgctgcatttctgacaggtatgaacgaatctaggtatcagcggttcttcgcgtggcgggatcggcttcgggtcaggttgtttacggattggagagagagattttgcgcaatatgcgataggtatcctcatctgcctagatcccaggtgtacgaagatttggaaggctggttccaagcatagaacgccatgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagagaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagtcggggtttcaccatattggccaggctggtgtccaactcctaatctcaggtgatctacccaccttggcctcccaaattgctgggattacaggcgtgaaccactgctcccttccctgtccttcagtggcatcgctcttttgcaatgctctttgggaaattatattagcctaattactaatttcctggccctcaaggtgacttgcttgaacttgccaccttcgccactagggggcagcattggtttacacagggtgaacaaacgttcaattctaaactaatattctttggtgggaaagtgtgttcattttggtttgttttgaagaagtcacttcatacctttgaactggggttaagctggttaactcccaaaattccaccagttccaatatcctatggaaaaaccccaaaaccactcaaaggatctgcgatcgctccggtgcccgtcagtgggcagagcgcacatcgcccacagtccccgagaagttggggggaggggtcggcaattgaacgggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagctgaagcttcgaggggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagtccgggcctttgtccggcgctcccttggagcctacctagactcagccggctctccacgctttgcctgaccctgcttgctcaactctacgtctttgtttcgttttctgttctgcgccgttacagatccaagctgtgaccggcgcctacgaatgccgccaccatgccccttcagctgttgttgcttttgatactgctgggacctggaaactcacttcagttgtgggacacatgggccgatgaggcggagaaggccctgggtccccttctcgcaagagacagacggcaagcgactgagtatgaatacctcgattatgatttcctccccgagactgagcctcctgagatgttgcgcaacagtacagacacaacgccattgactggccccggaactccagagagcaccacggttgagcctgctgcccggagaagtactggattggatgcgggcggcgctgtgactgaattgaccactgagctggccaatatggggaatctgtccacagacagcgcggccatggagatacaaacgacacagcctgcggccaccgaggcccagactacacagcctgtaccgacagaggcccaaactacaccattggcagctacggaagctcaaacaactcgattgaccgctaccgaggcacagactacccctctcgctgcgactgaagcccaaacgacaccacctgcagcaacagaagcacaaactacacagcccacagggttggaggcgcaaacaactgcgcctgctgccatggaagctcaaacaacggcaccggccgcaatggaggcacagactactcctcctgcagctatggaggcccaaacgacacagacgactgccatggaggcacagacaactgcaccagaggccacggaggcacaaacaacacaaccaactgcaacagaggcgcagacgacgcccctcgcagcaatggaagctctttccacggaacccagcgcaaccgaagcactctctatggagcctacaaccaaacggggcctttttattccattctctgtgtcatccgtgactcataaggggatacctatggctgcgtctaatctctcagtcaattatccagttggtgcgccagatcacatttccgtgaaacagtgtctccttgccatattgatacttgccttggtcgcgactatttttttcgtgtgtaccgtagtgctcgcggttcgcctctctcggaagggtcacatgtaccctgttaggaattattctcccactgaaatggtgtgcatcagcagtctgctgcctgatggtggcgagggaccatccgctacagccaacggaggactctctaaggctaagtctcctggactgacccctgagccccgcgaggatagggaaggtgacgatctcaccctccactcctttctgccatagaacgccatgaattcactcctcaggtgcaggctgcctatcagaaggtggtggctggtgtggccaatgccctggctcacaaataccactgagatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgcccatatgctggctgccatgaacaaaggttggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatggagatcagttcgctcgctcttttgcaatgctctttgggaaattatattagcctaattactaatttcctggccctcaaggtgacttgcttgaacttgccaccttcgccactagggggcagcattggtttacacagggtgaacaaacgttcaattctaaactaatattctttggtgggaaagtgtgttcattttggtttgttttgaagaagtcacttcatacctttgaactggggttaagctggttaactcccaaaattccaccagttccaatatcctatggaaaaaccccaaaaccactcaaaggatctgcgatcgctccggtgcccgtcagtgggcagagcgcacatcgcccacagtccccgagaagttggggggaggggtcggcaattgaacgggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagctgaagcttcgaggggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagtccgggcctttgtccggcgctcccttggagcctacctagactcagccggctctccacgctttgcctgaccctgcttgctcaactctacgtctttgtttcgttttctgttctgcgccgttacagatccaagctgtgaccggcgcctacgaatgccgccaccatggtgagcaaatctcgctggaaactgctagccatgctggctctcgtcttggtcgtgatggtatggtattccataagtagggaggacagatacatagaactcttctactttcccattccagagaagaaggaaccatgtctgcagggcgaggctgagtctaaagcttcaaagctgttcgggaactatagtcgagatcagcctattttcctgaggctcgaggactacttctgggtgaaaacccccagcgcatacgaactgccctatgggaccaagggatcagaggatctcttgctccgggtgctcgccatcacatcaagcagcattccaaaaaacatccaatccttgagatgccgtcggtgtgtagtggttggcaacggccataggctaagaaattcaagccttggtgatgcaattaacaaatacgacgtggtcatccgccttaataacgcccccgtggccggatatgagggcgatgtcggttccaaaacgactatgcggcttttttaccctgagtctgcacattttgaccctaaggtggagaataaccctgatacccttctggtcctggttgcctttaaggccatggacttccactggattgaaaccattctcagcgacaagaaacgcgttcggaaggggttctggaaacagccgcctctgatatgggatgttaatcccaagcaaatcaggatcttaaacccctttttcatggaaattgccgctgataagctgctgtccttaccaatgcagcagccgagaaagatcaagcagaaacctacaactggcttgttagcaataactttggctctgcacctgtgcgaccttgtgcatatcgcaggattcgggtacccagacgcgtataataagaagcagacaatccactactatgaacagatcaccctcaaatccatggctggtagtggccacaatgtgtcgcaagaggccctagccattaaacgaatgctggaaatgggagcgatcaagaatctgacatctttttaaaacgccatgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagagaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagtcggggtttcaccatattggccaggctggtgtccaactcctaatctcaggtgatctacccaccttggcctcccaaattgctgggattacaggcgtgaaccactgctcccttccctgtccttctgattttaaaataactataccagcaggaggacgtccagacacagcataggctacctggccatgcccaaccggtgggacatttgagttgcttgcttggcactgtcctctcatgcgttgggtccactcagtagatgcctgttcagtcagttcgctcttttgcaatgctctttgggaaattatattagcctaattactaatttcctggccctcaaggtgacttgcttgaacttgccaccttcgccactagggggcagcattggtttacacagggtgaacaaacgttcaattctaaactaatattctttggtgggaaagtgtgttcattttggtttgttttgaagaagtcacttcatacctttgaactggggttaagctggttaactcccaaaattccaccagttccaatatcctatggaaaaaccccaaaaccactcaaaggatctgccatcgctccggtgcccgtcagtgggcagagcgcacatcgcccacagtccccgagaagttggggggaggggtcggcaattgaacgggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagctgaagcttcgaggggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagtccgggcctttgtccggcgctcccttggagcctacctagactcagccggctctccacgctttgcctgaccctgcttgctcaactctacgtctttgtttcgttttctgttctgcgccgttacagatccaagctgtgaccggcgcctacgaatgccgccaccatggctgctgaaggttccgtcgccaggcagcctgacctcttgacctgcgacgatgagccgatccatatccccggtgccatccaaccgcatggactgctgctcgccctcgccgccgacatgacgatcgttgccggcagcgacaaccttcccgaactcaccggactggcgatcggcgccctgatcggccgctctgcggccgatgtgttcgactcggaaacgcacaaccgtctgacgatcgccttggccgagcccggggcggccgtcggagcaccgatcactgtcggcttcacgatgcgaaaggacgcaggcttcatcggctcctggcatcgccatgatcagctcatcttcctggagctggagcctccccagcgggacgtcgccgagccgcaggcgttcttccgccgcaccaacagcgccatccgccgcctgcaggccgccgaaaccttggaaagcgcctgcgccgccgcggcgcaagaggtgcggaagattaccggcttcgatcgggtgatgatctatcgcttcgcctccgacttcagcggcgaagtgatcgcagaggatcggtgcgccgaggtcgagtcaaaactaggcctgcactatcctgcctcaaccgtgccggcgcaggcccgtcggctctataccatcaacccggtacggatcattcccgatatcaattatcggccggtgccggtcaccccagacctcaatccggtcaccgggcggccgattgatcttagcttcgccatcctgcgcagcgtgtcgcccgtccatctggagttcatgcgcaacataggcatgcacggcacgatgtcgatctcgattttgcgcggcgagcgactgtggggattgatcgtttgccatcaccgaacgccgtactacgtcgatctcgatggccgccaagcctgcgagctagtcgcccaggttctggcctggcagatcggcgtgatggaagagtaaaacgbbabbabbabbabbabbaccatctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaaggcaatagcaggcatgctggggatgcggtgggctctatggcagttagggcggccgcgcctgggcttacttaattaacgagagcataatattgatatgtgccaaagttgtttctgactgactaataagtataatttgtttctattatgtataggttaagctaattacttattttataatacaacatgactgtttttaaagtacaaaataagtttatttttgtaaaagagagaatgtttaaaagttttgttactttatagaagaaattttgagtttttgtttttttttaataaataaataaacataaataaattgtttgttgaatttattattagtatgtaagtgtaaatataataaaacttaatatctattcaaattaataaataaacctcgatatacagaccgataaaacacatgcgtcaattttacgcatgattatctttaacgtacgtcacaatatgattatctttctagggttaaataatagtttctaatttttttattattcagcctgctgtcgtgaataccgagctccaattcgccctatagtgagtcgtattacaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttggg

SEQ ID NO: 39 - DNA, 16887 bp, synthetic construct
  source 1..16887: mol_type = other DNA
tgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctcgaaattaaccctcactaaagggaacaaaagctggtacctcgcgcgacttggtttgccattctttagcgcgcgtcgcgtcacacagcttggccacaatgtggtttttgtcaaacgaagattctatgacgtgtttaaagtttaggtcgagtaaagcgcaaatcttttttaaccctagaaagatagtctgcgtaaaattgacgcatgcattcttgaaatattgctctctctttctaaatagcgcgaatccgtcgctgtgcatttaggacatctcagtcgccgcttggagctcccgtgaggcgtgcttgtcaatgcggtaagtgtcactgattttgaactataacgaccgcgtgagtcaaaatgacgcatgattatcttttacgtgacttttaagatttaactcatacgataattatattgttatttcatgttctacttacgtgataacttattatatatatattttcttgttatagatatcatcaactttgtatagaaaagttgctcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattgcaagtttgtacaaaaaagcaggctgaatgccgccaccatggctaccgagtataaaccgacggtccgacttgcgactcgggatgacgttccacgcgccgtgaggactctggccgccgccttcgctgactacccggccacacggcacacagttgaccccgacagacacatagagcgggtcacagagttgcaagaacttttcctgacgcgggttggactcgatattggaaaagtttgggttgccgacgatggtgccgcagttgcggtgtggactactccagaaagtgtagaagctggagccgtattcgctgaaatagggccacggatggcagagctgtctggtagcaggctcgcagcgcagcagcagatggaaggccttctcgctccgcatcggcctaaggaaccggcgtggttcctggctacggtgggagtctcacctgaccatcaaggaaagggattgggaagtgctgtcgttcttccaggcgtggaagcggccgaacgagcaggcgtcccagccttcttggaaacgagcgcacccaggaatcttccattctacgagcgactgggtttcactgtgacagcagacgtagaagtccctgaagggcctcggacttggtgcatgaccagaaagcctggggcttgaaacgccatgatccagacatgataagatacattgatgagtttggacaaaccacaactagaatgcagtgaggcgcgcctggatacttctgcctgcaggggagtcgctcttttgcaatgctctttgggaaattatattagcctaattactaatttcctggccctcaaggtgacttgcttgaacttgccaccttcgccactagggggcagcattggtttacacagggtgaacaaacgttcaattctaaactaatattctttggtgggaaagtgtgttcattttggtttgttttgaagaagtcacttcatacctttgaactggggttaagctggttaactcccaaaattccaccagttccaatatcctatggaaaaaccccaaaaccactcaaaggatctgcgatcgctccggtgcccgtcagtgggcagagcgcacatcgcccacagtccccgagaagttggggggaggggtcggcaattgaacgggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagctgaagcttcgaggggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagtccgggcctttgtccggcgctcccttggagcctacctagactcagccggctctccacgctttgcctgaccctgcttgctcaactctacgtctttgtttcgttttctgttctgcgccgttacagatccaagctgtgaccggcgcctacgaatgccgccaccatgaaagatagctgtatcactgtcatggccatggccctgttgagcggcttcttcttttttgctccagcatcctcctataacctggacgttcgcggggcacggtccttttcccctcctagggccggcaggcattttggctaccgtgtgctgcaggtcggtaacggagtaatcgtgggtgccccgggagaaggaaacagcacgggaagtctgtaccagtgccagtctggtaccggccactgcttacccgtcaccctacgcgggagtaactacacatccaaatacctcggaatgactcttgcaacagatccgaccgatgggagcattttggcctgtgatccaggcctgtctcgaacctgcgaccaaaacacatacctgtccggactctgttacctctttcgccagaatttgcaggggccgatgctgcagggccggcccggatttcaggagtgtataaaaggcaatgtggacctagtatttctttttgacggaagcatgagcctgcaaccggatgagtttcagaagatcctagactttatgaaagatgttatgaagaagctctccaacacttcctatcagtttgccgcagtgcagttctccacctcctataagacggagtttgatttctctgactatgtgaaacgcaaagatcccgatgccctgctgaagcatgtgaagcatatgttgttgctgactaacacctttggggctataaactatgtggccaccgaggtgtttagggaggaattgggtgcccgccctgacgccaccaaggtccttattatcataacagacggcgaagccacagactcaggcaacattgatgctgcaaaagatataattcgatacattataggaatcggcaagcactttcagacaaaggagtctcaagagactctccataagtttgccagtaagcctgcttcagagttcgtaaaaattctggacactttcgaaaagctgaaagacctctttacagagctccagaagaaaatctatgtcatcgaaggcacttccaagcaggatctgactagcttcaacatggagctctcttcaagcggcatttcagctgatctctctcgcggtcacgcggtcgtgggggcggtaggagccaaagattgggctggtggcttcttggacctgaaggctgacctgcaagacgacacattcatcggtaatgaacccctaacgcccgaagtgagagccgggtacttgggctatactgtcacctggttgccatcgagacagaaaacctctctgttagccagcggagcgcccagataccaacacatgggtcgggttctcttgtttcaggaaccacaaggcggaggtcactggagccaggtgcagactatccatggaacccagatcggaagctacttcggcggggaattatgtggcgttgacgtggaccaggacggggagactgaactgcttctgatcggagcaccccttttctatggagagcagcggggaggaagagtgttcatctaccaacgtagacagctgggtttcgaagaggtttcagagcttcaaggcgatccaggctatcccctggggcgttttggcgaggcgatcacggctctgaccgatattaacggcgacggcctggtggacgttgcagttggggctccgctggaggaacagggtgccgtatacatattcaacggcagacacggaggactttctccccagccctcccagagaatcgagggaacacaggtcctgtctggcattcagtggttcggccggagtatccacggagtcaaggatcttgaaggcgatgggcttgcagacgtagccgtgggggcagaaagccagatgatcgttttgtcgtcaaggcctgtcgttgacatggtcactctgatgtccttttcccccgccgaaataccagtacacgaggtggagtgctcctattcgactagtaataagatgaaagagggtgtaaatatcacgatctgctttcagattaagtctcttattccacagttccaggggcgcctggtggccaatctcacctataccttacagcttgacgggcaccgtacacggcggaggggtctatttccaggcgggagacacgagctcaggaggaatatagcagtgacaacaagcatgagttgcaccgacttcagcttccacttccctgtctgcgtccaggatctgattagtcctatcaatgtctctctgaacttcagtctttgggaggaggaaggcacacctagagatcaaagggctcaggggaaggatatacctccaattttgcgaccctccctccacagcgaaacatgggaaatcccattcgaaaagaactgtggcgaagataagaaatgtgaggctaatctgcgagtttcatttagccccgccaggagccgcgctctgaggcttaccgccttcgcatcattaagtgtggaactatccctcagcaatctcgaggaggacgcctactgggtgcaactggatctccatttccctcccggactctcgtttcgaaaggtggaaatgctcaagcctcatagtcaaattccagtaagctgtgaggagctcccggaggagtctcgcctgttgtctcgagcattaagctgcaatgttagctcacccatcttcaaagccggccattcagtcgcactgcagatgatgttcaacaccctcgtgaactccagctggggtgactccgtcgaattacatgcaaatgttacctgcaataacgaggatagcgatctcctcgaggacaactccgctacgaccattatccctattttgtatcccatcaatattctcatccaggaccaagaggattcaacactgtacgtcagcttcaccccaaagggtccaaagattcaccaggtcaaacatatgtatcaggtgcggattcaaccttcaatacacgaccacaatataccaacattggaggccgtagtgggggtgccacagcccccttctgagggacctatcacccaccagtggtccgtgcagatggagccacccgttccatgccattatgaagatctggagcggctacctgacgctgccgaaccttgtttgcctggggcgctgtttagatgccccgtggtgttccggcaagaaattctggtgcaagtgatcggcactttagagcttgtgggggagattgaagcgtctagtatgttctctctgtgtagttctctgagcatctcattcaatagttctaagcattttcacctttacgggtcaaatgcgtcactggctcaagttgtgatgaaagtggacgtcgtgtacgaaaaacagatgttatacctgtacgtgctgagtggtatcggcggactgctactgctgctgcttattttcatcgtgctgtataaggtgggattctttaaaaggaacttaaaggaaaaaatggaagccgggagaggggttccgaacgggattcccgctgaggacagtgagcagctcgcatctggtcaggaggctggggacccagggtgcctcaaacctctgcatgaaaaagattcggagtccggcggcggcaaggattaaaacgccatgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagagaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagtcggggtttcaccatattggccaggctggtgtccaactcctaatctcaggtgatctacccaccttggcctcccaaattgctgggattacaggcgtgaaccactgctcccttccctgtccttcagtggcatcgctcttttgcaatgctctttgggaaattatattagcctaattactaatttcctggccctcaaggtgacttgcttgaacttgccaccttcgccactagggggcagcattggtttacacagggtgaacaaacgttcaattctaaactaatattctttggtgggaaagtgtgttcattttggtttgttttgaagaagtcacttcatacctttgaactggggttaagctggttaactcccaaaattccaccagttccaatatcctatggaaaaaccccaaaaccactcaaaggatctgcgatcgctccggtgcccgtcagtgggcagagcgcacatcgcccacagtccccgagaagttggggggaggggtcggcaattgaacgggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagctgaagcttcgaggggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagtccgggcctttgtccggcgctcccttggagcctacctagactcagccggctctccacgctttgcctgaccctgcttgctcaactctacgtctttgtttcgttttctgttctgcgccgttacagatccaagctgtgaccggcgcctacgaatgccgccaccatgttaggtctgcgccctccactgctggccctggtaggattactctccttggggtgtgttctcagccaggagtgcaccaaatttaaagtctccagctgccgggagtgtatcgagagcggcccaggctgcacatggtgccaaaaactcaacttcaccggaccaggagatcccgacagtataagatgtgatactaggcctcagctgttgatgcggggctgtgctgccgacgacattatggaccctaccagcttagcagaaacccaggaagatcataatggggggcaaaaacagctcagcccccagaaagtgaccctgtacctgcgacccggtcaagcggctgccttcaacgtgacttttcggcgcgcgaagggataccccatcgatctttattatctgatggatttgagctactcaatgctggatgacctgaggaacgtaaagaagttgggaggggatctgctgcgcgctcttaatgaaattacagaatcaggcaggatcggcttcggatcattcgtggacaaaacagtgctgccgttcgtgaacacacacccggacaagctaagaaatccttgccccaacaaggaaaaggagtgccagccgccatttgctttccgacacgtgctgaaactcacgaataactcaaatcaattccaaaccgaagtgggcaagcagctgatctctggcaacctggacgcccccgaggggggactagatgcgatgatgcaagttgccgcttgccccgaagagattggctggcgcaacgtgaccagactgctggtctttgcaactgatgacggcttccattttgctggcgatggaaagttgggggcaatcctgacacctaatgacggccgctgccatctagaagataacctctataagaggagtaatgagtttgactatccaagtgtcggccagctggcacataaactagccgagaacaacatccaaccgatcttcgcagtcacaagtaggatggtaaagacgtacgagaagctgactgagatcataccaaaatctgcagtgggggagctgagcgaagattctagcaatgttgtgcagcttattaagaatgcctataataagctttccagtagagtgtttctggaccacaatgcccttcccgacactctgaaagttacttacgattctttttgttccaacggcgtgacgcaccggaatcagcctcgcggtgactgtgacggcgtgcagatcaacgtgcctattacatttcaggttaaagtcactgccaccgagtgcatccaggagcaatcatttgtcatcagggctctcggtttcacggacattgtgaccgttcaggtgctccctcagtgcgaatgtcgatgcagagatcagagcagagataggtccttatgtcacgggaagggtttcttggaatgtgggatatgcagatgcgatacaggctatatagggaagaactgcgaatgccagacacagggccgatcgtcccaggagctggaggggagctgtcgcaaagataacaactctataatttgcagcgggctcggtgactgtgtgtgtggtcagtgtctctgccacactagtgacgtaccagggaaactcatttatggccagtactgcgagtgtgataccattaattgtgagaggtataacggacaggtctgcggcgggccaggccgtggactttgtttctgcggtaagtgccgatgccatcctggatttgaaggcagtgcttgtcaatgcgagcgtaccactgagggctgtttaaaccctcggcgtgtcgagtgctcgggacgcggtagatgccggtgtaacgtctgcgagtgccactctggttaccagctccccctctgtcaggaatgtcccggatgcccttctccttgcgggaagtatatttcctgtgcagaatgtctgaagtttgagaagggccccttcgggaagaactgctccgccgcgtgcccaggacttcagttatcaaataatccagtaaaagggagaacctgtaaggaaagggattccgaaggctgttgggtggcttacacactggagcagcaagacggtatggacagataccttatctacgtcgacgagtctcgggagtgtgtggccggaccaaatatcgctgccatcgtcggcggaacagtggccggaattgttctgatcgggatacttctactcgttatctggaaagcattgattcacttgtctgacctgcgtgaataccggcggttcgagaaagaaaagttgaaatcgcagtggaataatgacaatcccctctttaaatcagccaccactaccgtgatgaaccccaagttcgccgagtcctaaaacgccatgaattcactcctcaggtgcaggctgcctatcagaaggtggtggctggtgtggccaatgccctggctcacaaataccactgagatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgcccatatgctggctgccatgaacaaaggttggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatggagatcagttcgctcgctcttttgcaatgctctttgggaaattatattagcctaattactaatttcctggccctcaaggtgacttgcttgaacttgccaccttcgccactagggggcagcattggtttacacagggtgaacaaacgttcaattctaaactaatattctttggtgggaaagtgtgttcattttggtttgttttgaagaagtcacttcatacctttgaactggggttaagctggttaactcccaaaattccaccagttccaatatcctatggaaaaaccccaaaaccactcaaaggatctgccatcgctccggtgcccgtcagtgggcagagcgcacatcgcccacagtccccgagaagttggggggaggggtcggcaattgaacgggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagctgaagcttcgaggggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagtccgggcctttgtccggcgctcccttggagcctacctagactcagccggctctccacgctttgcctgaccctgcttgctcaactctacgtctttgtttcgttttctgttctgcgccgttacagatccaagctgtgaccggcgcctacgaatgccgccaccatggctgctgaaggttccgtcgccaggcagcctgacctcttgacctgcgacgatgagccgatccatatccccggtgccatccaaccgcatggactgctgctcgccctcgccgccgacatgacgatcgttgccggcagcgacaaccttcccgaactcaccggactggcgatcggcgccctgatcggccgctctgcggccgatgtgttcgactcggaaacgcacaaccgtctgacgatcgccttggccgagcccggggcggccgtcggagcaccgatcactgtcggcttcacgatgcgaaaggacgcaggcttcatcggctcctggcatcgccatgatcagctcatcttcctggagctggagcctccccagcgggacgtcgccgagccgcaggcgttcttccgccgcaccaacagcgccatccgccgcctgcaggccgccgaaaccttggaaagcgcctgcgccgccgcggcgcaagaggtgcggaagattaccggcttcgatcgggtgatgatctatcgcttcgcctccgacttcagcggcgaagtgatcgcagaggatcggtgcgccgaggtcgagtcaaaactaggcctgcactatcctgcctcaaccgtgccggcgcaggcccgtcggctctataccatcaacccggtacggatcattcccgatatcaattatcggccggtgccggtcaccccagacctcaatccggtcaccgggcggccgattgatcttagcttcgccatcctgcgcagcgtgtcgcccgtccatctggagttcatgcgcaacataggcatgcacggcacgatgtcgatctcgattttgcgcggcgagcgactgtggggattgatcgtttgccatcaccgaacgccgtactacgtcgatctcgatggccgccaagcctgcgagctagtcgcccaggttctggcctggcagatcggcgtgatggaagagtaaaacgbbabbabbabbabbabbaccatctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaaggcaatagcaggcatgctggggatgcggtgggctctatggcagtcagtgcggccgcgcctgggcttacttaattaacgagagcataatattgatatgtgccaaagttgtttctgactgactaataagtataatttgtttctattatgtataggttaagctaattacttattttataatacaacatgactgtttttaaagtacaaaataagtttatttttgtaaaagagagaatgtttaaaagttttgttactttatagaagaaattttgagtttttgtttttttttaataaataaataaacataaataaattgtttgttgaatttattattagtatgtaagtgtaaatataataaaacttaatatctattcaaattaataaataaacctcgatatacagaccgataaaacacatgcgtcaattttacgcatgattatctttaacgtacgtcacaatatgattatctttctagggttaaataatagtttctaatttttttattattcagcctgctgtcgtgaataccgagctccaattcgccctatagtgagtcgtattacaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttggg

SEQ ID NO: 40 - DNA, 15583 bp, synthetic construct
  source 1..15583: mol_type = other DNA
tgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctcgaaattaaccctcactaaagggaacaaaagctggtacctcgcgcgacttggtttgccattctttagcgcgcgtcgcgtcacacagcttggccacaatgtggtttttgtcaaacgaagattctatgacgtgtttaaagtttaggtcgagtaaagcgcaaatcttttttaaccctagaaagatagtctgcgtaaaattgacgcatgcattcttgaaatattgctctctctttctaaatagcgcgaatccgtcgctgtgcatttaggacatctcagtcgccgcttggagctcccgtgaggcgtgcttgtcaatgcggtaagtgtcactgattttgaactataacgaccgcgtgagtcaaaatgacgcatgattatcttttacgtgacttttaagatttaactcatacgataattatattgttatttcatgttctacttacgtgataacttattatatatatattttcttgttatagatatcatcaactttgtatagaaaagttgctcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattgcaagtttgtacaaaaaagcaggctgaatgccgccaccatggctaccgagtataaaccgacggtccgacttgcgactcgggatgacgttccacgcgccgtgaggactctggccgccgccttcgctgactacccggccacacggcacacagttgaccccgacagacacatagagcgggtcacagagttgcaagaacttttcctgacgcgggttggactcgatattggaaaagtttgggttgccgacgatggtgccgcagttgcggtgtggactactccagaaagtgtagaagctggagccgtattcgctgaaatagggccacggatggcagagctgtctggtagcaggctcgcagcgcagcagcagatggaaggccttctcgctccgcatcggcctaaggaaccggcgtggttcctggctacggtgggagtctcacctgaccatcaaggaaagggattgggaagtgctgtcgttcttccaggcgtggaagcggccgaacgagcaggcgtcccagccttcttggaaacgagcgcacccaggaatcttccattctacgagcgactgggtttcactgtgacagcagacgtagaagtccctgaagggcctcggacttggtgcatgaccagaaagcctggggcttgaaacgccatgatccagacatgataagatacattgatgagtttggacaaaccacaactagaatgcagtgaggcgcgcctggatacttctgcctgcaggggagtcgctcttttgcaatgctctttgggaaattatattagcctaattactaatttcctggccctcaaggtgacttgcttgaacttgccaccttcgccactagggggcagcattggtttacacagggtgaacaaacgttcaattctaaactaatattctttggtgggaaagtgtgttcattttggtttgttttgaagaagtcacttcatacctttgaactggggttaagctggttaactcccaaaattccaccagttccaatatcctatggaaaaaccccaaaaccactcaaaggatctgcgatcgctccggtgcccgtcagtgggcagagcgcacatcgcccacagtccccgagaagttggggggaggggtcggcaattgaacgggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagctgaagcttcgaggggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagtccgggcctttgtccggcgctcccttggagcctacctagactcagccggctctccacgctttgcctgaccctgcttgctcaactctacgtctttgtttcgttttctgttctgcgccgttacagatccaagctgtgaccggcgcctacgaatgccgccaccatgtggccgttggtggccgcacttctgctgggttctgcatgttgtgggagcgcgcagctccttttcaacaaaactaagagtgtggaatttacattttgcaacgatacagtcgtaatcccctgcttcgtgactaacatggaggcccaaaataccacagaggtttacgtgaaatggaagtttaagggcagggatatttataccttcgatggggctttgaacaagtctactgtgcctaccgacttcagctccgctaagattgaggtgagccagctgctcaagggcgacgcgagtctgaaaatggacaaatcagacgccgtcagtcatacgggaaattacacctgcgaagtcactgagctgacccgggaaggggagactataattgagttgaaatatcgagttgtctcctggttttcacccaatgaaaacatcctaattgttatattcccaatcttcgccattctgctgttctggggccagtttggaatcaagacgctaaaatatcgctcgggaggcatggatgaaaagaccatcgcactccttgttgccggcttggtgatcacggttatcgtgattgtgggagcgatcttatttgtgcctggcgagtatagcctcaaaaatgctacaggacttggtctgatagtaacatcaacagggattcttattctcttacactactacgtattttctaccgccatcggcctcacctccttcgtgatcgccatactcgtgattcaagtgatcgcctacatcctggctgtcgtcggcctgtctctgtgcatcgctgcctgtatccccatgcacgggccactgctgatttcaggactctccatactggctctggcccagttgctgggtctagtctacatgaagtttgtagcttccaatcagaaaactattcagccacccagaaaggcagtggaagagcctttaaatgcattcaaagaaagcaaggggatgatgaacgacgagtaaaacgccatgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagagaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagtcggggtttcaccatattggccaggctggtgtccaactcctaatctcaggtgatctacccaccttggcctcccaaattgctgggattacaggcgtgaaccactgctcccttccctgtccttcagtggcatcgctcttttgcaatgctctttgggaaattatattagcctaattactaatttcctggccctcaaggtgacttgcttgaacttgccaccttcgccactagggggcagcattggtttacacagggtgaacaaacgttcaattctaaactaatattctttggtgggaaagtgtgttcattttggtttgttttgaagaagtcacttcatacctttgaactggggttaagctggttaactcccaaaattccaccagttccaatatcctatggaaaaaccccaaaaccactcaaaggatctgcgatcgctccggtgcccgtcagtgggcagagcgcacatcgcccacagtccccgagaagttggggggaggggtcggcaattgaacgggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagctgaagcttcgaggggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagtccgggcctttgtccggcgctcccttggagcctacctagactcagccggctctccacgctttgcctgaccctgcttgctcaactctacgtctttgtttcgttttctgttctgcgccgttacagatccaagctgtgaccggcgcctacgaatgccgccaccatggtaggcagacgtgcgctgattgtgcttgcgcatagtgagagaacaagtttcaattatgcaatgaaagaggcagctgctgcagcgctcaagaaaaaagggtgggaggtggtagaatctgacctgtacgccatgaacttcaaccccattatttcccgcaaggacatcaccgggaaactgaaagatccagcaaactttcaatacccagctgagtctgtcctagcttacaaggaaggccatctcagtcccgatatagtggccgaacagaagaaactcgaagctgctgacttagtgatattccaattccccttgcagtggttcggcgttcccgccatcctgaagggttggtttgagagggtttttattggggagttcgcatacacttatgctgccatgtacgacaaggggccatttcgatcaaagaaagccgtcttgagcatcacaaccggagggtccggtagcatgtactctttacagggaatacacggcgatatgaatgtgatcctgtggcctattcagtcaggcatccttcacttctgcggatttcaggttctggagcctcagcttacatatagcatcggccacacgcctgccgatgcccggattcagattctcgagggttggaagaaaaggctggagaacatctgggacgaaaccccgctgtattttgccccaagctcgctgttcgatttgaactttcaggccggtttcctaatgaagaaggaagtgcaagacgaagagaaaaataagaagtttggactctccgtcggacatcacctgggcaagtccatccctactgataatcagatcaaggcccggaaataaaacgccatgaattcactcctcaggtgcaggctgcctatcagaaggtggtggctggtgtggccaatgccctggctcacaaataccactgagatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgcccatatgctggctgccatgaacaaaggttggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatggagatcagttcgctcgctcttttgcaatgctctttgggaaattatattagcctaattactaatttcctggccctcaaggtgacttgcttgaacttgccaccttcgccactagggggcagcattggtttacacagggtgaacaaacgttcaattctaaactaatattctttggtgggaaagtgtgttcattttggtttgttttgaagaagtcacttcatacctttgaactggggttaagctggttaactcccaaaattccaccagttccaatatcctatggaaaaaccccaaaaccactcaaaggatctgcgatcgctccggtgcccgtcagtgggcagagcgcacatcgcccacagtccccgagaagttggggggaggggtcggcaattgaacgggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagctgaagcttcgaggggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagtccgggcctttgtccggcgctcccttggagcctacctagactcagccggctctccacgctttgcctgaccctgcttgctcaactctacgtctttgtttcgttttctgttctgcgccgttacagatccaagctgtgaccggcgcctacgaatgccgccaccatgctggagggagtgcaggtggagactatctccccaggtgatggcagaacgttccctaagagaggacaaacatgcgtggtgcactacaccgggatgcttgaagatggaaagaaagttgattcctcccgggacagaaacaagccctttaagtttatgctaggcaagcaggaggtgatccgaggctgggaagaaggggttgcccagatgagtgtgggtcagagagccaaactgactatatctccagattatgcctatggtgccactgggcacccaggcatcatcccaccacatgccactctcgtgttcgatgtggagcttctaaaactggaatctggcggtggttccggagtcgacggatttggtgatgtcggtgctcttgagagtttgaggggaaatgcagatttggcttacatcctgagcatggagccctgtggccactgcctcattatcaacaatgtgaacttctgccgtgagtccgggctccgcacccgcactggctccaacatcgactgtgagaagttgcggcgtcgcttctcctcgctgcatttcatggtggaggtgaagggcgacctgactgccaagaaaatggtgctggctttgctggagcttgcgaggcaagatcatggggcattggactgctgcgtggtagtcattctctctcacggctgtcaggccagccacctgcagttcccaggggctgtctacggcacagatggatgccctgtgtcggtcgagaagattgtgaacatcttcaatgggaccagctgccccagcctgggagggaagcccaagctctttttcatccaggcctgtggtggggagcagaaagaccatgggtttgaggtggcctccacttcccctgaggacgagtcccctggcagtaaccccgagccagatgccaccccgttccaggaaggtttgaggaccttcgaccagctggacgccatatctagtttgcccacacccagtgacatctttgtgtcctactctactttcccaggttttgtttcctggagggaccccaagagtggctcctggtacgttgaaaccctggacgacatctttgagcagtgggctcactctgaagatctgcagtccctcctgcttagggtcgctaatgctgtttcggtgaaagggatttataaacagatgcctggttgctttaatttcctccggaaaaaacttttctttaaaacatcagtcgactatccgtatgacgtaccagactacgcactcgactaaaacgccatgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagagaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagtcggggtttcaccatattggccaggctggtgtccaactcctaatctcaggtgatctacccaccttggcctcccaaattgctgggattacaggcgtgaaccactgctcccttccctgtccttctgattttaaaataactataccagcaggaggacgtccagacacagcataggctacctggccatgcccaaccggtgggacatttgagttgcttgcttggcactgtcctctcatgcgttgggtccactcagtagatgcctgttcagtcagttcgctcttttgcaatgctctttgggaaattatattagcctaattactaatttcctggccctcaaggtgacttgcttgaacttgccaccttcgccactagggggcagcattggtttacacagggtgaacaaacgttcaattctaaactaatattctttggtgggaaagtgtgttcattttggtttgttttgaagaagtcacttcatacctttgaactggggttaagctggttaactcccaaaattccaccagttccaatatcctatggaaaaaccccaaaaccactcaaaggatctgccatcgctccggtgcccgtcagtgggcagagcgcacatcgcccacagtccccgagaagttggggggaggggtcggcaattgaacgggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagctgaagcttcgaggggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagtccgggcctttgtccggcgctcccttggagcctacctagactcagccggctctccacgctttgcctgaccctgcttgctcaactctacgtctttgtttcgttttctgttctgcgccgttacagatccaagctgtgaccggcgcctacgaatgccgccaccatggctgctgaaggttccgtcgccaggcagcctgacctcttgacctgcgacgatgagccgatccatatccccggtgccatccaaccgcatggactgctgctcgccctcgccgccgacatgacgatcgttgccggcagcgacaaccttcccgaactcaccggactggcgatcggcgccctgatcggccgctctgcggccgatgtgttcgactcggaaacgcacaaccgtctgacgatcgccttggccgagcccggggcggccgtcggagcaccgatcactgtcggcttcacgatgcgaaaggacgcaggcttcatcggctcctggcatcgccatgatcagctcatcttcctggagctggagcctccccagcgggacgtcgccgagccgcaggcgttcttccgccgcaccaacagcgccatccgccgcctgcaggccgccgaaaccttggaaagcgcctgcgccgccgcggcgcaagaggtgcggaagattaccggcttcgatcgggtgatgatctatcgcttcgcctccgacttcagcggcgaagtgatcgcagaggatcggtgcgccgaggtcgagtcaaaactaggcctgcactatcctgcctcaaccgtgccggcgcaggcccgtcggctctataccatcaacccggtacggatcattcccgatatcaattatcggccggtgccggtcaccccagacctcaatccggtcaccgggcggccgattgatcttagcttcgccatcctgcgcagcgtgtcgcccgtccatctggagttcatgcgcaacataggcatgcacggcacgatgtcgatctcgattttgcgcggcgagcgactgtggggattgatcgtttgccatcaccgaacgccgtactacgtcgatctcgatggccgccaagcctgcgagctagtcgcccaggttctggcctggcagatcggcgtgatggaagagtaaaacgbbabbabbabbabbabbaccatctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaaggcaatagcaggcatgctggggatgcggtgggctctatggcagttagggcggccgcgcctgggcttacttaattaacgagagcataatattgatatgtgccaaagttgtttctgactgactaataagtataatttgtttctattatgtataggttaagctaattacttattttataatacaacatgactgtttttaaagtacaaaataagtttatttttgtaaaagagagaatgtttaaaagttttgttactttatagaagaaattttgagtttttgtttttttttaataaataaataaacataaataaattgtttgttgaatttattattagtatgtaagtgtaaatataataaaacttaatatctattcaaattaataaataaacctcgatatacagaccgataaaacacatgcgtcaattttacgcatgattatctttaacgtacgtcacaatatgattatctttctagggttaaataatagtttctaatttttttattattcagcctgctgtcgtgaataccgagctccaattcgccctatagtgagtcgtattacaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttggg

SEQ ID NO: 41 - DNA, 15838 bp, synthetic construct
  source 1..15838: mol_type = other DNA
ttttatctctcattcgaaaattcccaacatcgggattatataactgaaaagttttggcgtaatgcgttggtcgcaggaaccgtacccgtagtcctcggaccaccccgcgcaacttacgaggcttttgttcccgcagacgcattcgtacacgtggacgatttcggttccgctcgggaactcgctgcatttctgacaggtatgaacgaatctaggtatcagcggttcttcgcgtggcgggatcggcttcgggtcaggttgtttacggattggagagagagattttgcgcaatatgcgataggtatcctcatctgcctagatcccaggtgtacgaagatttggaaggctggttccaagcatagaacgccatgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagagaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagtcggggtttcaccatattggccaggctggtgtccaactcctaatctcaggtgatctacccaccttggcctcccaaattgctgggattacaggcgtgaaccactgctcccttccctgtccttcagtggcatcgctcttttgcaatgctctttgggaaattatattagcctaattactaatttcctggccctcaaggtgacttgcttgaacttgccaccttcgccactagggggcagcattggtttacacagggtgaacaaacgttcaattctaaactaatattctttggtgggaaagtgtgttcattttggtttgttttgaagaagtcacttcatacctttgaactggggttaagctggttaactcccaaaattccaccagttccaatatcctatggaaaaaccccaaaaccactcaaaggatctgcgatcgctccggtgcccgtcagtgggcagagcgcacatcgcccacagtccccgagaagttggggggaggggtcggcaattgaacgggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagctgaagcttcgaggggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagtccgggcctttgtccggcgctcccttggagcctacctagactcagccggctctccacgctttgcctgaccctgcttgctcaactctacgtctttgtttcgttttctgttctgcgccgttacagatccaagctgtgaccggcgcctacgaatgccgccaccatgccccttcagctgttgttgcttttgatactgctgggacctggaaactcacttcagttgtgggacacatgggccgatgaggcggagaaggccctgggtccccttctcgcaagagacagacggcaagcgactgagtatgaatacctcgattatgatttcctccccgagactgagcctcctgagatgttgcgcaacagtacagacacaacgccattgactggccccggaactccagagagcaccacggttgagcctgctgcccggagaagtactggattggatgcgggcggcgctgtgactgaattgaccactgagctggccaatatggggaatctgtccacagacagcgcggccatggagatacaaacgacacagcctgcggccaccgaggcccagactacacagcctgtaccgacagaggcccaaactacaccattggcagctacggaagctcaaacaactcgattgaccgctaccgaggcacagactacccctctcgctgcgactgaagcccaaacgacaccacctgcagcaacagaagcacaaactacacagcccacagggttggaggcgcaaacaactgcgcctgctgccatggaagctcaaacaacggcaccggccgcaatggaggcacagactactcctcctgcagctatggaggcccaaacgacacagacgactgccatggaggcacagacaactgcaccagaggccacggaggcacaaacaacacaaccaactgcaacagaggcgcagacgacgcccctcgcagcaatggaagctctttccacggaacccagcgcaaccgaagcactctctatggagcctacaaccaaacggggcctttttattccattctctgtgtcatccgtgactcataaggggatacctatggctgcgtctaatctctcagtcaattatccagttggtgcgccagatcacatttccgtgaaacagtgtctccttgccatattgatacttgccttggtcgcgactatttttttcgtgtgtaccgtagtgctcgcggttcgcctctctcggaagggtcacatgtaccctgttaggaattattctcccactgaaatggtgtgcatcagcagtctgctgcctgatggtggcgagggaccatccgctacagccaacggaggactctctaaggctaagtctcctggactgacccctgagccccgcgaggatagggaaggtgacgatctcaccctccactcctttctgccatagaacgccatgaattcactcctcaggtgcaggctgcctatcagaaggtggtggctggtgtggccaatgccctggctcacaaataccactgagatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgcccatatgctggctgccatgaacaaaggttggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatggagatcagttcgctcgctcttttgcaatgctctttgggaaattatattagcctaattactaatttcctggccctcaaggtgacttgcttgaacttgccaccttcgccactagggggcagcattggtttacacagggtgaacaaacgttcaattctaaactaatattctttggtgggaaagtgtgttcattttggtttgttttgaagaagtcacttcatacctttgaactggggttaagctggttaactcccaaaattccaccagttccaatatcctatggaaaaaccccaaaaccactcaaaggatctgcgatcgctccggtgcccgtcagtgggcagagcgcacatcgcccacagtccccgagaagttggggggaggggtcggcaattgaacgggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagctgaagcttcgaggggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagtccgggcctttgtccggcgctcccttggagcctacctagactcagccggctctccacgctttgcctgaccctgcttgctcaactctacgtctttgtttcgttttctgttctgcgccgttacagatccaagctgtgaccggcgcctacgaatgccgccaccatggaaggaatttccatctatacctctgacaattatacagaagaaatgggaagtggagattacgatagcatgaaagagccatgctttcgcgaggagaacgcaaactttaacaagatatttctgcctacgatttattcaataatatttctgaccgggatagtcgggaacggtctggttatacttgtgatgggatatcaaaagaagctccgctcaatgacagataaatatcggcttcatctcagcgtcgctgatcttcttttcgtgattaccctgcctttctgggctgtggacgcagttgcaaattggtatttcggtaattttctgtgtaaagctgtgcacgttatatatactgttaatctgtattcctcagtgctgattctcgcatttattagcctcgataggtatctcgcgattgtccatgctaccaattcacaaagaccccgcaaacttcttgccgagaaagtagtgtacgtgggagtttggatacccgcgctgttgctcaccatacctgattttattttcgctaatgtgtctgaagctgacgatcgctacatttgcgatcggttttatccaaacgatctgtgggtcgtagttttccaattccaacatattatggtgggactcattcttccagggatcgtgattctctcttgttactgtatcattattagcaaattgagtcattcaaaagggcatcaaaagagaaaagcgttgaaaacaaccgtgattctgattcttgccttcttcgcgtgctggttgccgtattatatcggaatctctattgatagctttattcttctcgaaattatcaaacagggctgcgaatttgaaaatacggttcataagtggatcagcattacggaagctctcgccttctttcattgctgcctcaatccaattctgtacgcctttctgggcgctaagttcaagacgtccgctcaacatgcccttacttcagtctcacggggttcatcccttaaaattctgagcaaggggaaaaggggtggccacagctcagtgtcaaccgaatcagaatcatcttctttccatagctcttgaaacgccatgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagagaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagtcggggtttcaccatattggccaggctggtgtccaactcctaatctcaggtgatctacccaccttggcctcccaaattgctgggattacaggcgtgaaccactgctcccttccctgtccttctgattttaaaataactataccagcaggaggacgtccagacacagcataggctacctggccatgcccaaccggtgggacatttgagttgcttgcttggcactgtcctctcatgcgttgggtccactcagtagatgcctgttcagtcagttcgctcttttgcaatgctctttgggaaattatattagcctaattactaatttcctggccctcaaggtgacttgcttgaacttgccaccttcgccactagggggcagcattggtttacacagggtgaacaaacgttcaattctaaactaatattctttggtgggaaagtgtgttcattttggtttgttttgaagaagtcacttcatacctttgaactggggttaagctggttaactcccaaaattccaccagttccaatatcctatggaaaaaccccaaaaccactcaaaggatctgccatcgctccggtgcccgtcagtgggcagagcgcacatcgcccacagtccccgagaagttggggggaggggtcggcaattgaacgggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagctgaagcttcgaggggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagtccgggcctttgtccggcgctcccttggagcctacctagactcagccggctctccacgctttgcctgaccctgcttgctcaactctacgtctttgtttcgttttctgttctgcgccgttacagatccaagctgtgaccggcgcctacgaatgccgccaccatggctgctgaaggttccgtcgccaggcagcctgacctcttgacctgcgacgatgagccgatccatatccccggtgccatccaaccgcatggactgctgctcgccctcgccgccgacatgacgatcgttgccggcagcgacaaccttcccgaactcaccggactggcgatcggcgccctgatcggccgctctgcggccgatgtgttcgactcggaaacgcacaaccgtctgacgatcgccttggccgagcccggggcggccgtcggagcaccgatcactgtcggcttcacgatgcgaaaggacgcaggcttcatcggctcctggcatcgccatgatcagctcatcttcctggagctggagcctccccagcgggacgtcgccgagccgcaggcgttcttccgccgcaccaacagcgccatccgccgcctgcaggccgccgaaaccttggaaagcgcctgcgccgccgcggcgcaagaggtgcggaagattaccggcttcgatcgggtgatgatctatcgcttcgcctccgacttcagcggcgaagtgatcgcagaggatcggtgcgccgaggtcgagtcaaaactaggcctgcactatcctgcctcaaccgtgccggcgcaggcccgtcggctctataccatcaacccggtacggatcattcccgatatcaattatcggccggtgccggtcaccccagacctcaatccggtcaccgggcggccgattgatcttagcttcgccatcctgcgcagcgtgtcgcccgtccatctggagttcatgcgcaacataggcatgcacggcacgatgtcgatctcgattttgcgcggcgagcgactgtggggattgatcgtttgccatcaccgaacgccgtactacgtcgatctcgatggccgccaagcctgcgagctagtcgcccaggttctggcctggcagatcggcgtgatggaagagtaaaacgbbabbabbabbabbabbaccatctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaaggcaatagcaggcatgctggggatgcggtgggctctatggcagttagggcggccgcgcctgggcttacttaattaacgagagcataatattgatatgtgccaaagttgtttctgactgactaataagtataatttgtttctattatgtataggttaagctaattacttattttataatacaacatgactgtttttaaagtacaaaataagtttatttttgtaaaagagagaatgtttaaaagttttgttactttatagaagaaattttgagtttttgtttttttttaataaataaataaacataaataaattgtttgttgaatttattattagtatgtaagtgtaaatataataaaacttaatatctattcaaattaataaataaacctcgatatacagaccgataaaacacatgcgtcaattttacgcatgattatctttaacgtacgtcacaatatgattatctttctagggttaaataatagtttctaatttttttattattcagcctgctgtcgtgaataccgagctccaattcgccctatagtgagtcgtattacaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctcgaaattaaccctcactaaagggaacaaaagctggtacctcgcgcgacttggtttgccattctttagcgcgcgtcgcgtcacacagcttggccacaatgtggtttttgtcaaacgaagattctatgacgtgtttaaagtttaggtcgagtaaagcgcaaatcttttttaaccctagaaagatagtctgcgtaaaattgacgcatgcattcttgaaatattgctctctctttctaaatagcgcgaatccgtcgctgtgcatttaggacatctcagtcgccgcttggagctcccgtgaggcgtgcttgtcaatgcggtaagtgtcactgattttgaactataacgaccgcgtgagtcaaaatgacgcatgattatcttttacgtgacttttaagatttaactcatacgataattatattgttatttcatgttctacttacgtgataacttattatatatatattttcttgttatagatatcatcaactttgtatagaaaagttgctcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattgcaagtttgtacaaaaaagcaggctgaatgccgccaccatggctaccgagtataaaccgacggtccgacttgcgactcgggatgacgttccacgcgccgtgaggactctggccgccgccttcgctgactacccggccacacggcacacagttgaccccgacagacacatagagcgggtcacagagttgcaagaacttttcctgacgcgggttggactcgatattggaaaagtttgggttgccgacgatggtgccgcagttgcggtgtggactactccagaaagtgtagaagctggagccgtattcgctgaaatagggccacggatggcagagctgtctggtagcaggctcgcagcgcagcagcagatggaaggccttctcgctccgcatcggcctaaggaaccggcgtggttcctggctacggtgggagtctcacctgaccatcaaggaaagggattgggaagtgctgtcgttcttccaggcgtggaagcggccgaacgagcaggcgtcccagccttcttggaaacgagcgcacccaggaatcttccattctacgagcgactgggtttcactgtgacagcagacgtagaagtccctgaagggcctcggacttggtgcatgaccagaaagcctggggcttgaaacgccatgatccagacatgataagatacattgatgagtttggacaaaccacaactagaatgcagtgaggcgcgcctggatacttctgcctgcaggggagtcgctcttttgcaatgctctttgggaaattatattagcctaattactaatttcctggccctcaaggtgacttgcttgaacttgccaccttcgccactagggggcagcattggtttacacagggtgaacaaacgttcaattctaaactaatattctttggtgggaaagtgtgttcattttggtttgttttgaagaagtcacttcatacctttgaactggggttaagctggttaactcccaaaattccaccagttccaatatcctatggaaaaaccccaaaaccactcaaaggggttggggttgcgccttttccaaggcagccctgggtttgcgcagggacgcggctgctctgggcgtggttccgggaaacgcagcggcgccgaccctgggtctggcacattcttcacgtccgttcgcagcgtcacccggatcttcgccgctacccttgtgggccccccggcgacgcttcctgctccgcccctaagtcgggaaggttccttgcggttcgcggcgtgccggacgtgacaaacggaagccgcacgtctgactagtaccctcgcagacggacagcgccagggagcaatggcagcgcgccgaccgcgatgggctgtggccaatagcggctgctcagcagggcgcgccgagagcagcggccgggaaggggcggtgcgggaggcggggtgtggggcggtagtgtgggccctgttcctgcccgcgcggtgttccgcattctgcaagcctccggagcgcacgtcggcagtcggctccctcgttgaccgaatcaccgacctctctccccaggaatgccgccaccatgaataacgccggccacggaccaactagacgcttgaggggactcggagttttggctggagtcgcactcctggccgctctgtggctgctttggctccttggaagcgcgccaaggggcacacctgcaccacaacctaccataacaattctggtgtggcattggccatttaccgatcaaccacccgaattgccgtcagatacatgtaccaggtatgggattgcacggtgtcatctcagcgcgaatcgctcattgttggcttcagcagatgcggtagtgtttcatcatagagaacttcaaactcgcaggagccaccttcctttggcacaaaggccgcggggtcaaccgtgggtttgggcatcaatggaaagtccctcacatactcatgggctgtcacatctgcggggaatctttaattgggtactttcctatcgacgggatagcgatattttcgtcccatacggacgattggaacctcattggggtccttctccccctctccctgcaaaatccagagttgcagcttgggtcgtttccaattttcaagaacgccaacttagggctcgcctctatagacaactcgctccccaccttcgtgttgacgttttcggtcgagctaacgggcgcccgttgtgtgcgtcatgtctcgtacctacggttgctcaatatcgg

SEQ ID NO: 42 - DNA, 27048 bp, synthetic construct
  source 1..27048: mol_type = other DNA
agcagatgcggtagtgtttcatcatagagaacttcaaactcgcaggagccaccttcctttggcacaaaggccgcggggtcaaccgtgggtttgggcatcaatggaaagtccctcacatactcatgggctgtcacatctgcggggaatctttaattgggtactttcctatcgacgggatagcgatattttcgtcccatacggacgattggaacctcattggggtccttctccccctctccctgcaaaatccagagttgcagcttgggtcgtttccaattttcaagaacgccaacttagggctcgcctctatagacaactcgctccccaccttcgtgttgacgttttcggtcgagctaacgggcgcccgttgtgtgcgtcatgtctcgtacctacggttgctcaatatcggttttatctctcattcgaaaattcccaacatcgggattatataactgaaaagttttggcgtaatgcgttggtcgcaggaaccgtacccgtagtcctcggaccaccccgcgcaacttacgaggcttttgttcccgcagacgcattcgtacacgtggacgatttcggttccgctcgggaactcgctgcatttctgacaggtatgaacgaatctaggtatcagcggttcttcgcgtggcgggatcggcttcgggtcaggttgtttacggattggagagagagattttgcgcaatatgcgataggtatcctcatctgcctagatcccaggtgtacgaagatttggaaggctggttccaagcatagaacgccatgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagagaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagtcggggtttcaccatattggccaggctggtgtccaactcctaatctcaggtgatctacccaccttggcctcccaaattgctgggattacaggcgtgaaccactgctcccttccctgtccttcagtggcatcgctcttttgcaatgctctttgggaaattatattagcctaattactaatttcctggccctcaaggtgacttgcttgaacttgccaccttcgccactagggggcagcattggtttacacagggtgaacaaacgttcaattctaaactaatattctttggtgggaaagtgtgttcattttggtttgttttgaagaagtcacttcatacctttgaactggggttaagctggttaactcccaaaattccaccagttccaatatcctatggaaaaaccccaaaaccactcaaaggatctgcgatcgctccggtgcccgtcagtgggcagagcgcacatcgcccacagtccccgagaagttggggggaggggtcggcaattgaacgggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagctgaagcttcgaggggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagtccgggcctttgtccggcgctcccttggagcctacctagactcagccggctctccacgctttgcctgaccctgcttgctcaactctacgtctttgtttcgttttctgttctgcgccgttacagatccaagctgtgaccggcgcctacgaatgccgccaccatgccccttcagctgttgttgcttttgatactgctgggacctggaaactcacttcagttgtgggacacatgggccgatgaggcggagaaggccctgggtccccttctcgcaagagacagacggcaagcgactgagtatgaatacctcgattatgatttcctccccgagactgagcctcctgagatgttgcgcaacagtacagacacaacgccattgactggccccggaactccagagagcaccacggttgagcctgctgcccggagaagtactggattggatgcgggcggcgctgtgactgaattgaccactgagctggccaatatggggaatctgtccacagacagcgcggccatggagatacaaacgacacagcctgcggccaccgaggcccagactacacagcctgtaccgacagaggcccaaactacaccattggcagctacggaagctcaaacaactcgattgaccgctaccgaggcacagactacccctctcgctgcgactgaagcccaaacgacaccacctgcagcaacagaagcacaaactacacagcccacagggttggaggcgcaaacaactgcgcctgctgccatggaagctcaaacaacggcaccggccgcaatggaggcacagactactcctcctgcagctatggaggcccaaacgacacagacgactgccatggaggcacagacaactgcaccagaggccacggaggcacaaacaacacaaccaactgcaacagaggcgcagacgacgcccctcgcagcaatggaagctctttccacggaacccagcgcaaccgaagcactctctatggagcctacaaccaaacggggcctttttattccattctctgtgtcatccgtgactcataaggggatacctatggctgcgtctaatctctcagtcaattatccagttggtgcgccagatcacatttccgtgaaacagtgtctccttgccatattgatacttgccttggtcgcgactatttttttcgtgtgtaccgtagtgctcgcggttcgcctctctcggaagggtcacatgtaccctgttaggaattattctcccactgaaatggtgtgcatcagcagtctgctgcctgatggtggcgagggaccatccgctacagccaacggaggactctctaaggctaagtctcctggactgacccctgagccccgcgaggatagggaaggtgacgatctcaccctccactcctttctgccatagaacgccatgaattcactcctcaggtgcaggctgcctatcagaaggtggtggctggtgtggccaatgccctggctcacaaataccactgagatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgcccatatgctggctgccatgaacaaaggttggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatggagatcagttcgctcgctcttttgcaatgctctttgggaaattatattagcctaattactaatttcctggccctcaaggtgacttgcttgaacttgccaccttcgccactagggggcagcattggtttacacagggtgaacaaacgttcaattctaaactaatattctttggtgggaaagtgtgttcattttggtttgttttgaagaagtcacttcatacctttgaactggggttaagctggttaactcccaaaattccaccagttccaatatcctatggaaaaaccccaaaaccactcaaaggatctgcgatcgctccggtgcccgtcagtgggcagagcgcacatcgcccacagtccccgagaagttggggggaggggtcggcaattgaacgggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagctgaagcttcgaggggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagtccgggcctttgtccggcgctcccttggagcctacctagactcagccggctctccacgctttgcctgaccctgcttgctcaactctacgtctttgtttcgttttctgttctgcgccgttacagatccaagctgtgaccggcgcctacgaatgccgccaccatgctgtcaacatcccggtctagattcataagaaacaccaatgagagtggcgaagaggtgaccactttcttcgattatgattatggagccccctgtcataaatttgacgtcaaacagatcggtgcacagctgctgccacctctttatagcctcgtgtttattttcgggttcgtggggaatatgctcgtggttttgatcctcatcaactgcaagaagctcaaatgtctgaccgatatatacctactgaacctggccatttcagatctgctcttcctgattacgctgccactctgggcacattcggccgctaacgaatgggtgtttggtaacgctatgtgcaagctgttcacaggtctttatcatattggatacttcggcgggattttctttatcatcctgttgactattgaccgataccttgctatcgtacacgcagttttcgcccttaaggcgaggacagtcacctttggtgtagtgacatccgtcatcacttggttagtggccgttttcgctagcgtgcccgggattatctttactaaatgccagaaagaggattccgtgtatgtatgtgggccctacttcccgaggggctggaataactttcacacaattatgcgaaatatcctaggccttgtcttgcctcttctgatcatggtgatatgttattctggaatcctgaaaacattactcagatgccgcaatgaaaagaagaggcaccgggccgtgcgcgtcatttttaccatcatgattgtgtacttcctattttggacgccgtacaatatcgtgattttgttgaacaccttccaggagttttttggactgtctaactgcgaaagcacttcacagttagaccaggcgactcaagtgacggagacactgggcatgacccactgctgtataaatcccataatatacgccttcgtaggcgagaagtttcgtagtctgtttcacattgccttggggtgtcgcatcgcacctctccaaaagccagtttgcgggggaccaggcgtcagacctggcaaaaatgttaaggtcaccacccagggcctgctcgacggacgggggaagggtaaaagcatcggaagggcacccgaagcttccctgcaagacaaggagggcgcttagaacgccatgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagagaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagtcggggtttcaccatattggccaggctggtgtccaactcctaatctcaggtgatctacccaccttggcctcccaaattgctgggattacaggcgtgaaccactgctcccttccctgtccttctgattttaaaataactataccagcaggaggacgtccagacacagcataggctacctggccatgcccaaccggtgggacatttgagttgcttgcttggcactgtcctctcatgcgttgggtccactcagtagatgcctgttcagtcagttcgctcttttgcaatgctctttgggaaattatattagcctaattactaatttcctggccctcaaggtgacttgcttgaacttgccaccttcgccactagggggcagcattggtttacacagggtgaacaaacgttcaattctaaactaatattctttggtgggaaagtgtgttcattttggtttgttttgaagaagtcacttcatacctttgaactggggttaagctggttaactcccaaaattccaccagttccaatatcctatggaaaaaccccaaaaccactcaaaggatctgcgatcgctccggtgcccgtcagtgggcagagcgcacatcgcccacagtccccgagaagttggggggaggggtcggcaattgaacgggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagctgaagcttcgaggggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagtccgggcctttgtccggcgctcccttggagcctacctagactcagccggctctccacgctttgcctgaccctgcttgctcaactctacgtctttgtttcgttttctgttctgcgccgttacagatccaagctgtgaccggcgcctacgaatgccgccaccatgaaagatagctgtatcactgtcatggccatggccctgttgagcggcttcttcttttttgctccagcatcctcctataacctggacgttcgcggggcacggtccttttcccctcctagggccggcaggcattttggctaccgtgtgctgcaggtcggtaacggagtaatcgtgggtgccccgggagaaggaaacagcacgggaagtctgtaccagtgccagtctggtaccggccactgcttacccgtcaccctacgcgggagtaactacacatccaaatacctcggaatgactcttgcaacagatccgaccgatgggagcattttggcctgtgatccaggcctgtctcgaacctgcgaccaaaacacatacctgtccggactctgttacctctttcgccagaatttgcaggggccgatgctgcagggccggcccggatttcaggagtgtataaaaggcaatgtggacctagtatttctttttgacggaagcatgagcctgcaaccggatgagtttcagaagatcctagactttatgaaagatgttatgaagaagctctccaacacttcctatcagtttgccgcagtgcagttctccacctcctataagacggagtttgatttctctgactatgtgaaacgcaaagatcccgatgccctgctgaagcatgtgaagcatatgttgttgctgactaacacctttggggctataaactatgtggccaccgaggtgtttagggaggaattgggtgcccgccctgacgccaccaaggtccttattatcataacagacggcgaagccacagactcaggcaacattgatgctgcaaaagatataattcgatacattataggaatcggcaagcactttcagacaaaggagtctcaagagactctccataagtttgccagtaagcctgcttcagagttcgtaaaaattctggacactttcgaaaagctgaaagacctctttacagagctccagaagaaaatctatgtcatcgaaggcacttccaagcaggatctgactagcttcaacatggagctctcttcaagcggcatttcagctgatctctctcgcggtcacgcggtcgtgggggcggtaggagccaaagattgggctggtggcttcttggacctgaaggctgacctgcaagacgacacattcatcggtaatgaacccctaacgcccgaagtgagagccgggtacttgggctatactgtcacctggttgccatcgagacagaaaacctctctgttagccagcggagcgcccagataccaacacatgggtcgggttctcttgtttcaggaaccacaaggcggaggtcactggagccaggtgcagactatccatggaacccagatcggaagctacttcggcggggaattatgtggcgttgacgtggaccaggacggggagactgaactgcttctgatcggagcaccccttttctatggagagcagcggggaggaagagtgttcatctaccaacgtagacagctgggtttcgaagaggtttcagagcttcaaggcgatccaggctatcccctggggcgttttggcgaggcgatcacggctctgaccgatattaacggcgacggcctggtggacgttgcagttggggctccgctggaggaacagggtgccgtatacatattcaacggcagacacggaggactttctccccagccctcccagagaatcgagggaacacaggtcctgtctggcattcagtggttcggccggagtatccacggagtcaaggatcttgaaggcgatgggcttgcagacgtagccgtgggggcagaaagccagatgatcgttttgtcgtcaaggcctgtcgttgacatggtcactctgatgtccttttcccccgccgaaataccagtacacgaggtggagtgctcctattcgactagtaataagatgaaagagggtgtaaatatcacgatctgctttcagattaagtctcttattccacagttccaggggcgcctggtggccaatctcacctataccttacagcttgacgggcaccgtacacggcggaggggtctatttccaggcgggagacacgagctcaggaggaatatagcagtgacaacaagcatgagttgcaccgacttcagcttccacttccctgtctgcgtccaggatctgattagtcctatcaatgtctctctgaacttcagtctttgggaggaggaaggcacacctagagatcaaagggctcaggggaaggatatacctccaattttgcgaccctccctccacagcgaaacatgggaaatcccattcgaaaagaactgtggcgaagataagaaatgtgaggctaatctgcgagtttcatttagccccgccaggagccgcgctctgaggcttaccgccttcgcatcattaagtgtggaactatccctcagcaatctcgaggaggacgcctactgggtgcaactggatctccatttccctcccggactctcgtttcgaaaggtggaaatgctcaagcctcatagtcaaattccagtaagctgtgaggagctcccggaggagtctcgcctgttgtctcgagcattaagctgcaatgttagctcacccatcttcaaagccggccattcagtcgcactgcagatgatgttcaacaccctcgtgaactccagctggggtgactccgtcgaattacatgcaaatgttacctgcaataacgaggatagcgatctcctcgaggacaactccgctacgaccattatccctattttgtatcccatcaatattctcatccaggaccaagaggattcaacactgtacgtcagcttcaccccaaagggtccaaagattcaccaggtcaaacatatgtatcaggtgcggattcaaccttcaatacacgaccacaatataccaacattggaggccgtagtgggggtgccacagcccccttctgagggacctatcacccaccagtggtccgtgcagatggagccacccgttccatgccattatgaagatctggagcggctacctgacgctgccgaaccttgtttgcctggggcgctgtttagatgccccgtggtgttccggcaagaaattctggtgcaagtgatcggcactttagagcttgtgggggagattgaagcgtctagtatgttctctctgtgtagttctctgagcatctcattcaatagttctaagcattttcacctttacgggtcaaatgcgtcactggctcaagttgtgatgaaagtggacgtcgtgtacgaaaaacagatgttatacctgtacgtgctgagtggtatcggcggactgctactgctgctgcttattttcatcgtgctgtataaggtgggattctttaaaaggaacttaaaggaaaaaatggaagccgggagaggggttccgaacgggattcccgctgaggacagtgagcagctcgcatctggtcaggaggctggggacccagggtgcctcaaacctctgcatgaaaaagattcggagtccggcggcggcaaggattaaaacgccatgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagagaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagtcggggtttcaccatattggccaggctggtgtccaactcctaatctcaggtgatctacccaccttggcctcccaaattgctgggattacaggcgtgaaccactgctcccttccctgtccttcagttaggtcgctcttttgcaatgctctttgggaaattatattagcctaattactaatttcctggccctcaaggtgacttgcttgaacttgccaccttcgccactagggggcagcattggtttacacagggtgaacaaacgttcaattctaaactaatattctttggtgggaaagtgtgttcattttggtttgttttgaagaagtcacttcatacctttgaactggggttaagctggttaactcccaaaattccaccagttccaatatcctatggaaaaaccccaaaaccactcaaaggatctgcgatcgctccggtgcccgtcagtgggcagagcgcacatcgcccacagtccccgagaagttggggggaggggtcggcaattgaacgggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagctgaagcttcgaggggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagtccgggcctttgtccggcgctcccttggagcctacctagactcagccggctctccacgctttgcctgaccctgcttgctcaactctacgtctttgtttcgttttctgttctgcgccgttacagatccaagctgtgaccggcgcctacgaatgccgccaccatgttaggtctgcgccctccactgctggccctggtaggattactctccttggggtgtgttctcagccaggagtgcaccaaatttaaagtctccagctgccgggagtgtatcgagagcggcccaggctgcacatggtgccaaaaactcaacttcaccggaccaggagatcccgacagtataagatgtgatactaggcctcagctgttgatgcggggctgtgctgccgacgacattatggaccctaccagcttagcagaaacccaggaagatcataatggggggcaaaaacagctcagcccccagaaagtgaccctgtacctgcgacccggtcaagcggctgccttcaacgtgacttttcggcgcgcgaagggataccccatcgatctttattatctgatggatttgagctactcaatgctggatgacctgaggaacgtaaagaagttgggaggggatctgctgcgcgctcttaatgaaattacagaatcaggcaggatcggcttcggatcattcgtggacaaaacagtgctgccgttcgtgaacacacacccggacaagctaagaaatccttgccccaacaaggaaaaggagtgccagccgccatttgctttccgacacgtgctgaaactcacgaataactcaaatcaattccaaaccgaagtgggcaagcagctgatctctggcaacctggacgcccccgaggggggactagatgcgatgatgcaagttgccgcttgccccgaagagattggctggcgcaacgtgaccagactgctggtctttgcaactgatgacggcttccattttgctggcgatggaaagttgggggcaatcctgacacctaatgacggccgctgccatctagaagataacctctataagaggagtaatgagtttgactatccaagtgtcggccagctggcacataaactagccgagaacaacatccaaccgatcttcgcagtcacaagtaggatggtaaagacgtacgagaagctgactgagatcataccaaaatctgcagtgggggagctgagcgaagattctagcaatgttgtgcagcttattaagaatgcctataataagctttccagtagagtgtttctggaccacaatgcccttcccgacactctgaaagttacttacgattctttttgttccaacggcgtgacgcaccggaatcagcctcgcggtgactgtgacggcgtgcagatcaacgtgcctattacatttcaggttaaagtcactgccaccgagtgcatccaggagcaatcatttgtcatcagggctctcggtttcacggacattgtgaccgttcaggtgctccctcagtgcgaatgtcgatgcagagatcagagcagagataggtccttatgtcacgggaagggtttcttggaatgtgggatatgcagatgcgatacaggctatatagggaagaactgcgaatgccagacacagggccgatcgtcccaggagctggaggggagctgtcgcaaagataacaactctataatttgcagcgggctcggtgactgtgtgtgtggtcagtgtctctgccacactagtgacgtaccagggaaactcatttatggccagtactgcgagtgtgataccattaattgtgagaggtataacggacaggtctgcggcgggccaggccgtggactttgtttctgcggtaagtgccgatgccatcctggatttgaaggcagtgcttgtcaatgcgagcgtaccactgagggctgtttaaaccctcggcgtgtcgagtgctcgggacgcggtagatgccggtgtaacgtctgcgagtgccactctggttaccagctccccctctgtcaggaatgtcccggatgcccttctccttgcgggaagtatatttcctgtgcagaatgtctgaagtttgagaagggccccttcgggaagaactgctccgccgcgtgcccaggacttcagttatcaaataatccagtaaaagggagaacctgtaaggaaagggattccgaaggctgttgggtggcttacacactggagcagcaagacggtatggacagataccttatctacgtcgacgagtctcgggagtgtgtggccggaccaaatatcgctgccatcgtcggcggaacagtggccggaattgttctgatcgggatacttctactcgttatctggaaagcattgattcacttgtctgacctgcgtgaataccggcggttcgagaaagaaaagttgaaatcgcagtggaataatgacaatcccctctttaaatcagccaccactaccgtgatgaaccccaagttcgccgagtcctaaaacgccatgaattcactcctcaggtgcaggctgcctatcagaaggtggtggctggtgtggccaatgccctggctcacaaataccactgagatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgcccatatgctggctgccatgaacaaaggttggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatggagatcagtgaatgcggccgctcgctcttttgcaatgctctttgggaaattatattagcctaattactaatttcctggccctcaaggtgacttgcttgaacttgccaccttcgccactagggggcagcattggtttacacagggtgaacaaacgttcaattctaaactaatattctttggtgggaaagtgtgttcattttggtttgttttgaagaagtcacttcatacctttgaactggggttaagctggttaactcccaaaattccaccagttccaatatcctatggaaaaaccccaaaaccactcaaaggatctgcgatcgctccggtgcccgtcagtgggcagagcgcacatcgcccacagtccccgagaagttggggggaggggtcggcaattgaacgggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagctgaagcttcgaggggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagtccgggcctttgtccggcgctcccttggagcctacctagactcagccggctctccacgctttgcctgaccctgcttgctcaactctacgtctttgtttcgttttctgttctgcgccgttacagatccaagctgtgaccggcgcctacgaatgccgccaccatgctgtcaacatcccggtctagattcataagaaacaccaatgagagtggcgaagaggtgaccactttcttcgattatgattatggagccccctgtcataaatttgacgtcaaacagatcggtgcacagctgctgccacctctttatagcctcgtgtttattttcgggttcgtggggaatatgctcgtggttttgatcctcatcaactgcaagaagctcaaatgtctgaccgatatatacctactgaacctggccatttcagatctgctcttcctgattacgctgccactctgggcacattcggccgctaacgaatgggtgtttggtaacgctatgtgcaagctgttcacaggtctttatcatattggatacttcggcgggattttctttatcatcctgttgactattgaccgataccttgctatcgtacacgcagttttcgcccttaaggcgaggacagtcacctttggtgtagtgacatccgtcatcacttggttagtggccgttttcgctagcgtgcccgggattatctttactaaatgccagaaagaggattccgtgtatgtatgtgggccctacttcccgaggggctggaataactttcacacaattatgcgaaatatcctaggccttgtcttgcctcttctgatcatggtgatatgttattctggaatcctgaaaacattactcagatgccgcaatgaaaagaagaggcaccgggccgtgcgcgtcatttttaccatcatgattgtgtacttcctattttggacgccgtacaatatcgtgattttgttgaacaccttccaggagttttttggactgtctaactgcgaaagcacttcacagttagaccaggcgactcaagtgacggagacactgggcatgacccactgctgtataaatcccataatatacgccttcgtaggcgagaagtttcgtagtctgtttcacattgccttggggtgtcgcatcgcacctctccaaaagccagtttgcgggggaccaggcgtcagacctggcaaaaatgttaaggtcaccacccagggcctgctcgacggacgggggaagggtaaaagcatcggaagggcacccgaagcttccctgcaagacaaggagggcgcttagaacgccatgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagagaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagtcggggtttcaccatattggccaggctggtgtccaactcctaatctcaggtgatctacccaccttggcctcccaaattgctgggattacaggcgtgaaccactgctcccttccctgtccttctgattttaaaataactataccagcaggaggacgtccagacacagcataggctacctggccatgcccaaccggtgggacatttgagttgcttgcttggcactgtcctctcatgcgttgggtccactcagtagatgcctgttcagtggatcctcgctcttttgcaatgctctttgggaaattatattagcctaattactaatttcctggccctcaaggtgacttgcttgaacttgccaccttcgccactagggggcagcattggtttacacagggtgaacaaacgttcaattctaaactaatattctttggtgggaaagtgtgttcattttggtttgttttgaagaagtcacttcatacctttgaactggggttaagctggttaactcccaaaattccaccagttccaatatcctatggaaaaaccccaaaaccactcaaaggatctgccatcgctccggtgcccgtcagtgggcagagcgcacatcgcccacagtccccgagaagttggggggaggggtcggcaattgaacgggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagctgaagcttcgaggggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagtccgggcctttgtccggcgctcccttggagcctacctagactcagccggctctccacgctttgcctgaccctgcttgctcaactctacgtctttgtttcgttttctgttctgcgccgttacagatccaagctgtgaccggcgcctacgaatgccgccaccatggctgctgaaggttccgtcgccaggcagcctgacctcttgacctgcgacgatgagccgatccatatccccggtgccatccaaccgcatggactgctgctcgccctcgccgccgacatgacgatcgttgccggcagcgacaaccttcccgaactcaccggactggcgatcggcgccctgatcggccgctctgcggccgatgtgttcgactcggaaacgcacaaccgtctgacgatcgccttggccgagcccggggcggccgtcggagcaccgatcactgtcggcttcacgatgcgaaaggacgcaggcttcatcggctcctggcatcgccatgatcagctcatcttcctggagctggagcctccccagcgggacgtcgccgagccgcaggcgttcttccgccgcaccaacagcgccatccgccgcctgcaggccgccgaaaccttggaaagcgcctgcgccgccgcggcgcaagaggtgcggaagattaccggcttcgatcgggtgatgatctatcgcttcgcctccgacttcagcggcgaagtgatcgcagaggatcggtgcgccgaggtcgagtcaaaactaggcctgcactatcctgcctcaaccgtgccggcgcaggcccgtcggctctataccatcaacccggtacggatcattcccgatatcaattatcggccggtgccggtcaccccagacctcaatccggtcaccgggcggccgattgatcttagcttcgccatcctgcgcagcgtgtcgcccgtccatctggagttcatgcgcaacataggcatgcacggcacgatgtcgatctcgattttgcgcggcgagcgactgtggggattgatcgtttgccatcaccgaacgccgtactacgtcgatctcgatggccgccaagcctgcgagctagtcgcccaggttctggcctggcagatcggcgtgatggaagagtaaaacgbbabbabbabbabbabbaccatctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaaggcaatagcaggcatgctggggatgcggtgggctctatggcagtttaattaacgagagcataatattgatatgtgccaaagttgtttctgactgactaataagtataatttgtttctattatgtataggttaagctaattacttattttataatacaacatgactgtttttaaagtacaaaataagtttatttttgtaaaagagagaatgtttaaaagttttgttactttatagaagaaattttgagtttttgtttttttttaataaataaataaacataaataaattgtttgttgaatttattattagtatgtaagtgtaaatataataaaacttaatatctattcaaattaataaataaacctcgatatacagaccgataaaacacatgcgtcaattttacgcatgattatctttaacgtacgtcacaatatgattatctttctagggttaaataatagtttctaatttttttattattcagcctgctgtcgtgaataccgagctccaattcgccctatagtgagtcgtattacaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctcgaaattaaccctcactaaagggaacaaaagctggtacctcgcgcgacttggtttgccattctttagcgcgcgtcgcgtcacacagcttggccacaatgtggtttttgtcaaacgaagattctatgacgtgtttaaagtttaggtcgagtaaagcgcaaatcttttttaaccctagaaagatagtctgcgtaaaattgacgcatgcattcttgaaatattgctctctctttctaaatagcgcgaatccgtcgctgtgcatttaggacatctcagtcgccgcttggagctcccgtgaggcgtgcttgtcaatgcggtaagtgtcactgattttgaactataacgaccgcgtgagtcaaaatgacgcatgattatcttttacgtgacttttaagatttaactcatacgataattatattgttatttcatgttctacttacgtgataacttattatatatatattttcttgttatagatatcatcaactttgtatagaaaagttgctcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattgcaagtttgtacaaaaaagcaggctgaatgccgccaccatggctaccgagtataaaccgacggtccgacttgcgactcgggatgacgttccacgcgccgtgaggactctggccgccgccttcgctgactacccggccacacggcacacagttgaccccgacagacacatagagcgggtcacagagttgcaagaacttttcctgacgcgggttggactcgatattggaaaagtttgggttgccgacgatggtgccgcagttgcggtgtggactactccagaaagtgtagaagctggagccgtattcgctgaaatagggccacggatggcagagctgtctggtagcaggctcgcagcgcagcagcagatggaaggccttctcgctccgcatcggcctaaggaaccggcgtggttcctggctacggtgggagtctcacctgaccatcaaggaaagggattgggaagtgctgtcgttcttccaggcgtggaagcggccgaacgagcaggcgtcccagccttcttggaaacgagcgcacccaggaatcttccattctacgagcgactgggtttcactgtgacagcagacgtagaagtccctgaagggcctcggacttggtgcatgaccagaaagcctggggcttgaaacgccatgatccagacatgataagatacattgatgagtttggacaaaccacaactagaatgcagtgaggcgcgcctggatacttctgcctgcaggggagtcgctcttttgcaatgctctttgggaaattatattagcctaattactaatttcctggccctcaaggtgacttgcttgaacttgccaccttcgccactagggggcagcattggtttacacagggtgaacaaacgttcaattctaaactaatattctttggtgggaaagtgtgttcattttggtttgttttgaagaagtcacttcatacctttgaactggggttaagctggttaactcccaaaattccaccagttccaatatcctatggaaaaaccccaaaaccactcaaaggggttggggttgcgccttttccaaggcagccctgggtttgcgcagggacgcggctgctctgggcgtggttccgggaaacgcagcggcgccgaccctgggtctggcacattcttcacgtccgttcgcagcgtcacccggatcttcgccgctacccttgtgggccccccggcgacgcttcctgctccgcccctaagtcgggaaggttccttgcggttcgcggcgtgccggacgtgacaaacggaagccgcacgtctgactagtaccctcgcagacggacagcgccagggagcaatggcagcgcgccgaccgcgatgggctgtggccaatagcggctgctcagcagggcgcgccgagagcagcggccgggaaggggcggtgcgggaggcggggtgtggggcggtagtgtgggccctgttcctgcccgcgcggtgttccgcattctgcaagcctccggagcgcacgtcggcagtcggctccctcgttgaccgaatcaccgacctctctccccaggaatgccgccaccatgaataacgccggccacggaccaactagacgcttgaggggactcggagttttggctggagtcgcactcctggccgctctgtggctgctttggctccttggaagcgcgccaaggggcacacctgcaccacaacctaccataacaattctggtgtggcattggccatttaccgatcaaccacccgaattgccgtcagatacatgtaccaggtatgggattgcacggtgtcatctcagcgcgaatcgctcattgttggcttc

SEQ ID NO: 43 - DNA, 19492 bp, synthetic construct
  source 1..19492: mol_type = other DNA
ttttttaaccctagaaagatagtctgcgtaaaattgacgcatgcattcttgaaatattgctctctctttctaaatagcgcgaatccgtcgctgtgcatttaggacatctcagtcgccgcttggagctcccgtgaggcgtgcttgtcaatgcggtaagtgtcactgattttgaactataacgaccgcgtgagtcaaaatgacgcatgattatcttttacgtgacttttaagatttaactcatacgataattatattgttatttcatgttctacttacgtgataacttattatatatatattttcttgttatagatatcatcaactttgtatagaaaagttgctcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattgcaagtttgtacaaaaaagcaggctgaatgccgccaccatggctaccgagtataaaccgacggtccgacttgcgactcgggatgacgttccacgcgccgtgaggactctggccgccgccttcgctgactacccggccacacggcacacagttgaccccgacagacacatagagcgggtcacagagttgcaagaacttttcctgacgcgggttggactcgatattggaaaagtttgggttgccgacgatggtgccgcagttgcggtgtggactactccagaaagtgtagaagctggagccgtattcgctgaaatagggccacggatggcagagctgtctggtagcaggctcgcagcgcagcagcagatggaaggccttctcgctccgcatcggcctaaggaaccggcgtggttcctggctacggtgggagtctcacctgaccatcaaggaaagggattgggaagtgctgtcgttcttccaggcgtggaagcggccgaacgagcaggcgtcccagccttcttggaaacgagcgcacccaggaatcttccattctacgagcgactgggtttcactgtgacagcagacgtagaagtccctgaagggcctcggacttggtgcatgaccagaaagcctggggcttgaaacgccatgatccagacatgataagatacattgatgagtttggacaaaccacaactagaatgcagtgaggcgcgcctggatacttctgcctgcaggggagtcgctcttttgcaatgctctttgggaaattatattagcctaattactaatttcctggccctcaaggtgacttgcttgaacttgccaccttcgccactagggggcagcattggtttacacagggtgaacaaacgttcaattctaaactaatattctttggtgggaaagtgtgttcattttggtttgttttgaagaagtcacttcatacctttgaactggggttaagctggttaactcccaaaattccaccagttccaatatcctatggaaaaaccccaaaaccactcaaaggatctgcgatcgctccggtgcccgtcagtgggcagagcgcacatcgcccacagtccccgagaagttggggggaggggtcggcaattgaacgggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagctgaagcttcgaggggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagtccgggcctttgtccggcgctcccttggagcctacctagactcagccggctctccacgctttgcctgaccctgcttgctcaactctacgtctttgtttcgttttctgttctgcgccgttacagatccaagctgtgaccggcgcctacgaatgccgccaccatgaaagatagctgtatcactgtcatggccatggccctgttgagcggcttcttcttttttgctccagcatcctcctataacctggacgttcgcggggcacggtccttttcccctcctagggccggcaggcattttggctaccgtgtgctgcaggtcggtaacggagtaatcgtgggtgccccgggagaaggaaacagcacgggaagtctgtaccagtgccagtctggtaccggccactgcttacccgtcaccctacgcgggagtaactacacatccaaatacctcggaatgactcttgcaacagatccgaccgatgggagcattttggcctgtgatccaggcctgtctcgaacctgcgaccaaaacacatacctgtccggactctgttacctctttcgccagaatttgcaggggccgatgctgcagggccggcccggatttcaggagtgtataaaaggcaatgtggacctagtatttctttttgacggaagcatgagcctgcaaccggatgagtttcagaagatcctagactttatgaaagatgttatgaagaagctctccaacacttcctatcagtttgccgcagtgcagttctccacctcctataagacggagtttgatttctctgactatgtgaaacgcaaagatcccgatgccctgctgaagcatgtgaagcatatgttgttgctgactaacacctttggggctataaactatgtggccaccgaggtgtttagggaggaattgggtgcccgccctgacgccaccaaggtccttattatcataacagacggcgaagccacagactcaggcaacattgatgctgcaaaagatataattcgatacattataggaatcggcaagcactttcagacaaaggagtctcaagagactctccataagtttgccagtaagcctgcttcagagttcgtaaaaattctggacactttcgaaaagctgaaagacctctttacagagctccagaagaaaatctatgtcatcgaaggcacttccaagcaggatctgactagcttcaacatggagctctcttcaagcggcatttcagctgatctctctcgcggtcacgcggtcgtgggggcggtaggagccaaagattgggctggtggcttcttggacctgaaggctgacctgcaagacgacacattcatcggtaatgaacccctaacgcccgaagtgagagccgggtacttgggctatactgtcacctggttgccatcgagacagaaaacctctctgttagccagcggagcgcccagataccaacacatgggtcgggttctcttgtttcaggaaccacaaggcggaggtcactggagccaggtgcagactatccatggaacccagatcggaagctacttcggcggggaattatgtggcgttgacgtggaccaggacggggagactgaactgcttctgatcggagcaccccttttctatggagagcagcggggaggaagagtgttcatctaccaacgtagacagctgggtttcgaagaggtttcagagcttcaaggcgatccaggctatcccctggggcgttttggcgaggcgatcacggctctgaccgatattaacggcgacggcctggtggacgttgcagttggggctccgctggaggaacagggtgccgtatacatattcaacggcagacacggaggactttctccccagccctcccagagaatcgagggaacacaggtcctgtctggcattcagtggttcggccggagtatccacggagtcaaggatcttgaaggcgatgggcttgcagacgtagccgtgggggcagaaagccagatgatcgttttgtcgtcaaggcctgtcgttgacatggtcactctgatgtccttttcccccgccgaaataccagtacacgaggtggagtgctcctattcgactagtaataagatgaaagagggtgtaaatatcacgatctgctttcagattaagtctcttattccacagttccaggggcgcctggtggccaatctcacctataccttacagcttgacgggcaccgtacacggcggaggggtctatttccaggcgggagacacgagctcaggaggaatatagcagtgacaacaagcatgagttgcaccgacttcagcttccacttccctgtctgcgtccaggatctgattagtcctatcaatgtctctctgaacttcagtctttgggaggaggaaggcacacctagagatcaaagggctcaggggaaggatatacctccaattttgcgaccctccctccacagcgaaacatgggaaatcccattcgaaaagaactgtggcgaagataagaaatgtgaggctaatctgcgagtttcatttagccccgccaggagccgcgctctgaggcttaccgccttcgcatcattaagtgtggaactatccctcagcaatctcgaggaggacgcctactgggtgcaactggatctccatttccctcccggactctcgtttcgaaaggtggaaatgctcaagcctcatagtcaaattccagtaagctgtgaggagctcccggaggagtctcgcctgttgtctcgagcattaagctgcaatgttagctcacccatcttcaaagccggccattcagtcgcactgcagatgatgttcaacaccctcgtgaactccagctggggtgactccgtcgaattacatgcaaatgttacctgcaataacgaggatagcgatctcctcgaggacaactccgctacgaccattatccctattttgtatcccatcaatattctcatccaggaccaagaggattcaacactgtacgtcagcttcaccccaaagggtccaaagattcaccaggtcaaacatatgtatcaggtgcggattcaaccttcaatacacgaccacaatataccaacattggaggccgtagtgggggtgccacagcccccttctgagggacctatcacccaccagtggtccgtgcagatggagccacccgttccatgccattatgaagatctggagcggctacctgacgctgccgaaccttgtttgcctggggcgctgtttagatgccccgtggtgttccggcaagaaattctggtgcaagtgatcggcactttagagcttgtgggggagattgaagcgtctagtatgttctctctgtgtagttctctgagcatctcattcaatagttctaagcattttcacctttacgggtcaaatgcgtcactggctcaagttgtgatgaaagtggacgtcgtgtacgaaaaacagatgttatacctgtacgtgctgagtggtatcggcggactgctactgctgctgcttattttcatcgtgctgtataaggtgggattctttaaaaggaacttaaaggaaaaaatggaagccgggagaggggttccgaacgggattcccgctgaggacagtgagcagctcgcatctggtcaggaggctggggacccagggtgcctcaaacctctgcatgaaaaagattcggagtccggcggcggcaaggattaaaacgccatgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagagaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagtcggggtttcaccatattggccaggctggtgtccaactcctaatctcaggtgatctacccaccttggcctcccaaattgctgggattacaggcgtgaaccactgctcccttccctgtccttcagtggcatcgctcttttgcaatgctctttgggaaattatattagcctaattactaatttcctggccctcaaggtgacttgcttgaacttgccaccttcgccactagggggcagcattggtttacacagggtgaacaaacgttcaattctaaactaatattctttggtgggaaagtgtgttcattttggtttgttttgaagaagtcacttcatacctttgaactggggttaagctggttaactcccaaaattccaccagttccaatatcctatggaaaaaccccaaaaccactcaaaggatctgcgatcgctccggtgcccgtcagtgggcagagcgcacatcgcccacagtccccgagaagttggggggaggggtcggcaattgaacgggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagctgaagcttcgaggggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagtccgggcctttgtccggcgctcccttggagcctacctagactcagccggctctccacgctttgcctgaccctgcttgctcaactctacgtctttgtttcgttttctgttctgcgccgttacagatccaagctgtgaccggcgcctacgaatgccgccaccatgttaggtctgcgccctccactgctggccctggtaggattactctccttggggtgtgttctcagccaggagtgcaccaaatttaaagtctccagctgccgggagtgtatcgagagcggcccaggctgcacatggtgccaaaaactcaacttcaccggaccaggagatcccgacagtataagatgtgatactaggcctcagctgttgatgcggggctgtgctgccgacgacattatggaccctaccagcttagcagaaacccaggaagatcataatggggggcaaaaacagctcagcccccagaaagtgaccctgtacctgcgacccggtcaagcggctgccttcaacgtgacttttcggcgcgcgaagggataccccatcgatctttattatctgatggatttgagctactcaatgctggatgacctgaggaacgtaaagaagttgggaggggatctgctgcgcgctcttaatgaaattacagaatcaggcaggatcggcttcggatcattcgtggacaaaacagtgctgccgttcgtgaacacacacccggacaagctaagaaatccttgccccaacaaggaaaaggagtgccagccgccatttgctttccgacacgtgctgaaactcacgaataactcaaatcaattccaaaccgaagtgggcaagcagctgatctctggcaacctggacgcccccgaggggggactagatgcgatgatgcaagttgccgcttgccccgaagagattggctggcgcaacgtgaccagactgctggtctttgcaactgatgacggcttccattttgctggcgatggaaagttgggggcaatcctgacacctaatgacggccgctgccatctagaagataacctctataagaggagtaatgagtttgactatccaagtgtcggccagctggcacataaactagccgagaacaacatccaaccgatcttcgcagtcacaagtaggatggtaaagacgtacgagaagctgactgagatcataccaaaatctgcagtgggggagctgagcgaagattctagcaatgttgtgcagcttattaagaatgcctataataagctttccagtagagtgtttctggaccacaatgcccttcccgacactctgaaagttacttacgattctttttgttccaacggcgtgacgcaccggaatcagcctcgcggtgactgtgacggcgtgcagatcaacgtgcctattacatttcaggttaaagtcactgccaccgagtgcatccaggagcaatcatttgtcatcagggctctcggtttcacggacattgtgaccgttcaggtgctccctcagtgcgaatgtcgatgcagagatcagagcagagataggtccttatgtcacgggaagggtttcttggaatgtgggatatgcagatgcgatacaggctatatagggaagaactgcgaatgccagacacagggccgatcgtcccaggagctggaggggagctgtcgcaaagataacaactctataatttgcagcgggctcggtgactgtgtgtgtggtcagtgtctctgccacactagtgacgtaccagggaaactcatttatggccagtactgcgagtgtgataccattaattgtgagaggtataacggacaggtctgcggcgggccaggccgtggactttgtttctgcggtaagtgccgatgccatcctggatttgaaggcagtgcttgtcaatgcgagcgtaccactgagggctgtttaaaccctcggcgtgtcgagtgctcgggacgcggtagatgccggtgtaacgtctgcgagtgccactctggttaccagctccccctctgtcaggaatgtcccggatgcccttctccttgcgggaagtatatttcctgtgcagaatgtctgaagtttgagaagggccccttcgggaagaactgctccgccgcgtgcccaggacttcagttatcaaataatccagtaaaagggagaacctgtaaggaaagggattccgaaggctgttgggtggcttacacactggagcagcaagacggtatggacagataccttatctacgtcgacgagtctcgggagtgtgtggccggaccaaatatcgctgccatcgtcggcggaacagtggccggaattgttctgatcgggatacttctactcgttatctggaaagcattgattcacttgtctgacctgcgtgaataccggcggttcgagaaagaaaagttgaaatcgcagtggaataatgacaatcccctctttaaatcagccaccactaccgtgatgaaccccaagttcgccgagtcctaaaacgccatgaattcactcctcaggtgcaggctgcctatcagaaggtggtggctggtgtggccaatgccctggctcacaaataccactgagatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgcccatatgctggctgccatgaacaaaggttggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatggagatcagttcgctcgctcttttgcaatgctctttgggaaattatattagcctaattactaatttcctggccctcaaggtgacttgcttgaacttgccaccttcgccactagggggcagcattggtttacacagggtgaacaaacgttcaattctaaactaatattctttggtgggaaagtgtgttcattttggtttgttttgaagaagtcacttcatacctttgaactggggttaagctggttaactcccaaaattccaccagttccaatatcctatggaaaaaccccaaaaccactcaaaggatctgcgatcgctccggtgcccgtcagtgggcagagcgcacatcgcccacagtccccgagaagttggggggaggggtcggcaattgaacgggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagctgaagcttcgaggggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagtccgggcctttgtccggcgctcccttggagcctacctagactcagccggctctccacgctttgcctgaccctgcttgctcaactctacgtctttgtttcgttttctgttctgcgccgttacagatccaagctgtgaccggcgcctacgaatgccgccaccatgctgtcaacatcccggtctagattcataagaaacaccaatgagagtggcgaagaggtgaccactttcttcgattatgattatggagccccctgtcataaatttgacgtcaaacagatcggtgcacagctgctgccacctctttatagcctcgtgtttattttcgggttcgtggggaatatgctcgtggttttgatcctcatcaactgcaagaagctcaaatgtctgaccgatatatacctactgaacctggccatttcagatctgctcttcctgattacgctgccactctgggcacattcggccgctaacgaatgggtgtttggtaacgctatgtgcaagctgttcacaggtctttatcatattggatacttcggcgggattttctttatcatcctgttgactattgaccgataccttgctatcgtacacgcagttttcgcccttaaggcgaggacagtcacctttggtgtagtgacatccgtcatcacttggttagtggccgttttcgctagcgtgcccgggattatctttactaaatgccagaaagaggattccgtgtatgtatgtgggccctacttcccgaggggctggaataactttcacacaattatgcgaaatatcctaggccttgtcttgcctcttctgatcatggtgatatgttattctggaatcctgaaaacattactcagatgccgcaatgaaaagaagaggcaccgggccgtgcgcgtcatttttaccatcatgattgtgtacttcctattttggacgccgtacaatatcgtgattttgttgaacaccttccaggagttttttggactgtctaactgcgaaagcacttcacagttagaccaggcgactcaagtgacggagacactgggcatgacccactgctgtataaatcccataatatacgccttcgtaggcgagaagtttcgtagtctgtttcacattgccttggggtgtcgcatcgcacctctccaaaagccagtttgcgggggaccaggcgtcagacctggcaaaaatgttaaggtcaccacccagggcctgctcgacggacgggggaagggtaaaagcatcggaagggcacccgaagcttccctgcaagacaaggagggcgcttagaacgccatgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagagaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagtcggggtttcaccatattggccaggctggtgtccaactcctaatctcaggtgatctacccaccttggcctcccaaattgctgggattacaggcgtgaaccactgctcccttccctgtccttctgattttaaaataactataccagcaggaggacgtccagacacagcataggctacctggccatgcccaaccggtgggacatttgagttgcttgcttggcactgtcctctcatgcgttgggtccactcagtagatgcctgttcagtcagttcgctcttttgcaatgctctttgggaaattatattagcctaattactaatttcctggccctcaaggtgacttgcttgaacttgccaccttcgccactagggggcagcattggtttacacagggtgaacaaacgttcaattctaaactaatattctttggtgggaaagtgtgttcattttggtttgttttgaagaagtcacttcatacctttgaactggggttaagctggttaactcccaaaattccaccagttccaatatcctatggaaaaaccccaaaaccactcaaaggatctgccatcgctccggtgcccgtcagtgggcagagcgcacatcgcccacagtccccgagaagttggggggaggggtcggcaattgaacgggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagctgaagcttcgaggggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagtccgggcctttgtccggcgctcccttggagcctacctagactcagccggctctccacgctttgcctgaccctgcttgctcaactctacgtctttgtttcgttttctgttctgcgccgttacagatccaagctgtgaccggcgcctacgaatgccgccaccatggctgctgaaggttccgtcgccaggcagcctgacctcttgacctgcgacgatgagccgatccatatccccggtgccatccaaccgcatggactgctgctcgccctcgccgccgacatgacgatcgttgccggcagcgacaaccttcccgaactcaccggactggcgatcggcgccctgatcggccgctctgcggccgatgtgttcgactcggaaacgcacaaccgtctgacgatcgccttggccgagcccggggcggccgtcggagcaccgatcactgtcggcttcacgatgcgaaaggacgcaggcttcatcggctcctggcatcgccatgatcagctcatcttcctggagctggagcctccccagcgggacgtcgccgagccgcaggcgttcttccgccgcaccaacagcgccatccgccgcctgcaggccgccgaaaccttggaaagcgcctgcgccgccgcggcgcaagaggtgcggaagattaccggcttcgatcgggtgatgatctatcgcttcgcctccgacttcagcggcgaagtgatcgcagaggatcggtgcgccgaggtcgagtcaaaactaggcctgcactatcctgcctcaaccgtgccggcgcaggcccgtcggctctataccatcaacccggtacggatcattcccgatatcaattatcggccggtgccggtcaccccagacctcaatccggtcaccgggcggccgattgatcttagcttcgccatcctgcgcagcgtgtcgcccgtccatctggagttcatgcgcaacataggcatgcacggcacgatgtcgatctcgattttgcgcggcgagcgactgtggggattgatcgtttgccatcaccgaacgccgtactacgtcgatctcgatggccgccaagcctgcgagctagtcgcccaggttctggcctggcagatcggcgtgatggaagagtaaaacgbbabbabbabbabbabbaccatctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaaggcaatagcaggcatgctggggatgcggtgggctctatggcagttagggcggccgcgcctgggcttacttaattaacgagagcataatattgatatgtgccaaagttgtttctgactgactaataagtataatttgtttctattatgtataggttaagctaattacttattttataatacaacatgactgtttttaaagtacaaaataagtttatttttgtaaaagagagaatgtttaaaagttttgttactttatagaagaaattttgagtttttgtttttttttaataaataaataaacataaataaattgtttgttgaatttattattagtatgtaagtgtaaatataataaaacttaatatctattcaaattaataaataaacctcgatatacagaccgataaaacacatgcgtcaattttacgcatgattatctttaacgtacgtcacaatatgattatctttctagggttaaataatagtttctaatttttttattattcagcctgctgtcgtgaataccgagctccaattcgccctatagtgagtcgtattacaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctcgaaattaaccctcactaaagggaacaaaagctggtacctcgcgcgacttggtttgccattctttagcgcgcgtcgcgtcacacagcttggccacaatgtggtttttgtcaaacgaagattctatgacgtgtttaaagtttaggtcgagtaaagcgcaaatc

SEQ ID NO: 44 - DNA, 7378 bp, synthetic construct
  source 1..7378: mol_type = other DNA
gcgcaaatcttttttaaccctagaaagatagtctgcgtaaaattgacgcatgcattcttgaaatattgctctctctttctaaatagcgcgaatccgtcgctgtgcatttaggacatctcagtcgccgcttggagctcccgtgaggcgtgcttgtcaatgcggtaagtgtcactgattttgaactataacgaccgcgtgagtcaaaatgacgcatgattatcttttacgtgacttttaagatttaactcatacgataattatattgttatttcatgttctacttacgtgataacttattatatatatattttcttgttatagatatcatcaactttgtatagaaaagttgctcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattgcaagtttgtacaaaaaagcaggctgaatgccgccaccatggccaagcctttgtctcaagaagaatccaccctcattgaaagagcaacggctacaatcaacagcatccccatctctgaggactacagcgtcgccagcgcagctctctctagcgacggccgcatcttcactggtgtcaatgtatatcattttactgggggaccttgtgcagaactcgtggtgctgggcactgctgctgctgcggcagctggcaacctgacttgtatcgtcgcgatcggaaatgagaacaggggcatcttgagcccctgcggacggtgccgacaggtgcttctcgatctgcatcctgggatcaaagccatagtgaaggacagtgatggacagccgacggcagttgggattcgtgaattgctgccctctggttatgtgtgggagggctaaaacgccatgatccagacatgataagatacattgatgagtttggacaaaccacaactagaatgcagtgaggcgcgcctggatacttctgcctgcaggggagatgtcttccaaattgacggctagctcagtcctaggtacagtgctagcataccagataagttcgtcggtcggttatgcagtttaaggtttacacctataaaagagagagccgttatcgtctgtttgtggatgtacagagtgatattattgacacgcccgggcgacggatggtgatccccctggccagtgcacgtctgctgtcagataaagtctcccgtgaactttacccggtggtgcatatcggggatgaaagctggcgcatgatgaccaccgatatggccagtgtgccggtgtccgttatcggggaagaagtggctgatctcagccaccgcgaaaatgacatcaaaaacgccattaacctgatgttctggggaatataataatggcctcggtaccaaattccagaaaagaggcctcccgaaaggggggccttttttcgttttggtccgtggcggaccaaaacgaaaaaagacgcttttcagcgtcttattgttcgtctttggtaccgaggccattatttgtatagttcatccatgccaccggtagaatgacgaccctccgcgcgctcatattgctctacgatcgtataatcttcattatgagataagatgtccagtttaatatccacattgtacgcgccaggcagctgcacaggtttcttggctttgtacgtggttttcacttcagcgtcataatggccgccatctttcagtttcaggcgctgtttaatttcgcctttcagcgcaccatcttccggatacatgcgttcggtagacgcctcccaacccatcgtcctacactgcatcaccgggccatcagatggaaaattagtaccacgcagtttaactttatagatgaactcgccatcctgaagggaggagtcctgagtgacggtcacgacaccaccatcttcaaaattcattacgcgttcccagttgaaaccttccggaaaagacagcttcagatagtccgggatatccgctgggtgtttaacatacgctttagaaccgtacataaattgcgggctcaggatgtcccacgcaaaaggcagcgggccgcctttagtcactttcagtttggcggtctgggtgccttcatacggacggccctcgccttcgccttcgatctcgaactcgtgaccgttaacagaaccctccatgtgaactttgaagcgcatgaactctttaatgatagctaagttgttttcttctcctttactcaccatttagtatttctcctctttcactaggtcgagtagctagcataatacctaggactgagctagctgtaaagaaggaagacatcagtgcggccgcgcctgggcttacttaattaacgagagcataatattgatatgtgccaaagttgtttctgactgactaataagtataatttgtttctattatgtataggttaagctaattacttattttataatacaacatgactgtttttaaagtacaaaataagtttatttttgtaaaagagagaatgtttaaaagttttgttactttatagaagaaattttgagtttttgtttttttttaataaataaataaacataaataaattgtttgttgaatttattattagtatgtaagtgtaaatataataaaacttaatatctattcaaattaataaataaacctcgatatacagaccgataaaacacatgcgtcaattttacgcatgattatctttaacgtacgtcacaatatgattatctttctagggttaaataatagtttctaatttttttattattcagcctgctgtcgtgaataccgagctccaattcgccctatagtgagtcgtattacaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctcgaaattaaccctcactaaagggaacaaaagctggtacctcgcgcgacttggtttgccattctttagcgcgcgtcgcgtcacacagcttggccacaatgtggtttttgtcaaacgaagattctatgacgtgtttaaagtttaggtcgagtaaa

SEQ ID NO: 45 - DNA, 831 bp, synthetic construct
  source 1..831: mol_type = other DNA
atgatcattcttatttatctgtttctgctgttgtgggaggatactcaaggatggggtttcaaggatggcatcttccacaactccatctggctggagcgcgctgccggggtttaccacagagaggctaggtctgggaagtacaaactcacttatgccgaagctaaggcagtgtgtgaattcgaaggtggtcatttggccacatacaagcagctggaggcggccaggaaaattggattccacgtctgcgcagccgggtggatggctaaaggccgcgtgggctatccgatagtcaagcccggacccaactgcgggttcggaaagacaggcatcattgattacggcattcgtctgaatagatcagaaagatgggacgcatactgttacaatcctcatgcaaaagagtgcggaggagtgtttaccgaccccaagcaaatatttaaaagccctggtttcccaaacgagtatgaggataatcagatatgttactggcatatccgactcaagtacggacagcggattcacctcagcttcctggatttcgatcttgaagatgacccagggtgtctcgccgactacgtagagatctacgacagttatgacgacgtgcatggtttcgtgggccggtattgcggcgacgaattgccagatgatattatcagcacagggaatgtgatgacgcttaagtttctatctgacgctagcgtcaccgcgggggggtttcagatcaagtatgtagccatggaccctgtttccaaaagttcacagggcaaaaacacatcgaccacttccaccggcaataaaaactttctggccggcaggttttctcactta

SEQ ID NO: 46 - DNA, 681 bp, synthetic construct
  source 1..681: mol_type = other DNA
atggctcacgctgcacaggtggggctgcaggatgcaactagccctatcatggaggagctgattacatttcatgaccatgcactgatgataattttcttaatctgttttcttgtgctgtatgccctattcttgactctgacgaccaagttgacaaacaccaatatcagcgacgcccaggaaatggagactgtctggaccatactccccgccattattttagtgctcatagccctgccatccctgcgcatcttgtacatgaccgatgaagtcaatgacccatcactcactattaaatctatcggtcaccagtggtactggacatatgagtacacagattacggcggtctgatctttaactcttatatgttaccgcccctctttctggagccgggcgaccttagactcctggacgttgataaccgtgtagtcctgcccattgaagcccctatacgaatgatgatcaccagtcaggatgttctgcattcatgggctgtgccaacgctgggactcaaaactgacgcgattcccggcaggcttaaccaaaccaccttcacagctacacggcctggggtgtactatgggcaatgctccgagatctgcggagccaatcactcgttcatgccaattgtattggaactaatccccctcaagatcttcgaaatgggacctgtgtttaccctt

SEQ ID NO: 47 - DNA, 1209 bp, synthetic construct
  source 1..1209: mol_type = other DNA
atggcccatgccatggagaatagctggaccatctctaaggaatatcacattgacgaagaggtaggattcgcccttcctaaccctcaggaaaatctgccagacttttacaacgattggatgtttatcgctaagcaccttcccgatctgatcgaatcgggacagctgagagaaagggtcgagaaactgaatatgctgtcaattgaccacctgactgaccacaaaagtcagcggctcgcacgcttggtactgggttgcatcacgatggcatatgtctggggtaaaggacatggcgatgtgagaaaggttctgccacggaacatagccgtgccctattgtcagctgtccaagaaacttgagctgcccccaatcctggtttacgcggattgcgtgctagctaactggaagaagaaagacccaaacaaacccctgacctatgagaacatggatgtgttatttagctttcgagatggggattgttcaaagggcttcttccttgtgtcccttctggtcgagatagccgccgcatccgccatcaaagtgatccctaccgtttttaaagctatgcagatgcaggaacgcgacactctcctcaaagccttattggaaatcgcttcttgcttggagaaagccttacaggtgtttcaccaaattcatgaccatgtgaatcctaaagcgttcttcagcgtgctcaggatttacctaagcggctggaaggggaatccccagctgtcggatggcctcgtatacgaaggcttttgggaggacccaaaggagttcgcaggtggatcagctgggcaatcttctgtgttccaatgcttcgatgttctgctggggattcagcagactgcaggtggcggccatgctgcacagttcctccaggacatgcgcaggtacatgcctccagctcacagaaattttctctgtagtcttgaaagtaacccgtccgttcgtgagtttgtcttgagtaagggcgatgcaggactccgggaggcgtacgacgcctgtgtcaaagccctggtgtccttgcgaagctaccacttacaaatagtgaccaagtatattttgattcccgcttcacagcaaccgaaggaaaacaagacaagcgaggacccttccaaactcgaggccaagggaacgggggggacagacctcatgaatttcctaaagacagtcaggtctacaaccgagaagagcctgctgaaggaaggc

SEQ ID NO: 48 - DNA, 669 bp, synthetic construct
  source 1..669: mol_type = other DNA
atggcctgcctggggtttcagaggcacaaggcccagctgaatctcgcaacgcgtacatggccttgcaccctgctgttcttcctgttgtttatcccagttttttgtaaggccatgcatgttgcccagccagcagtggtcctcgcatcatcgagagggatcgcttcatttgtgtgcgagtatgctagtccaggcaaagccacagaagtccgagtgacagtattgagacaggctgacagtcaggtcactgaggtctgcgctgccacttatatgatgggaaatgaacttaccttcctcgacgattctatttgtaccggtaccagcagcggcaaccaggtaaacctaacaatacaaggcctccgcgcgatggacacgggtctatatatttgtaaagtggagctcatgtaccctcccccttactacctgggcattggaaatggcacacaaatctacgtgatagatccggaaccttgtcccgattctgactttcttctgtggattcttgcagcggtttccagcggattattcttctatagctttctgttgaccgccgtgtccctgtctaagatgttaaagaagcggtcccccctgaccactggggtgtacgtgaaaatgccacccactgaacctgagtgcgagaaacagttccaaccctacttcatcccgatcaac

SEQ ID NO: 49 - DNA, 1014 bp, synthetic construct
  source 1..1014: mol_type = other DNA
atggtggtgatggcccctaggacccttttcctgcttttgagtggggcactgacactgaccgaaacgtgggccggtagtcattccatgcgatatttctcagcagccgtcagcagacccggaagaggagagcctcgcttcattgccatgggttacgtcgatgatacacagttcgtacggtttgattctgattccgcttgccctaggatggagccgcgagcaccatgggtggaacaggagggccctgaatattgggaggaggagacacggaataccaaggctcacgcccagactgataggatgaacttacaaaccctgagggggtactacaatcaaagcgaagcttcatcgcacaccctgcagtggatgatcggctgcgatctggggtcagacgggcgcctcttgcggggctacgagcagtatgcatacgacggcaaagactatctcgctctgaatgaggacctgcgaagttggacagctgcggataccgccgcccagatctccaagagaaaatgtgaagccgcaaacgtcgcggagcagagaagggcttacctcgagggaacgtgtgtcgaatggctccacagatatctagagaacggcaaggagatgctccagcgggccgacccaccaaaaacccacgtgacccatcatcccgtttttgactatgaggccactctgcgttgctgggcccttggcttttacccagctgaaatcattctaacttggcaacgcgacggtgaggatcagacacaagacgtggaactcgttgaaacgcggccggcaggcgacggcacttttcagaaatgggccgctgtcgtggttccctccggagaggaacaacgttacacttgtcacgtgcagcatgaaggactgccagagccccttatgttacgctggaagcagtctagcctgcccacaatccctataatgggaattgtagcggggttggttgtgctggcagcagtggtaactggggccgcggtggctgccgtgttgtggcgcaagaagtctagcgac